(19) United States
(12) Patent Application Publication
(10) Pub. No.: US 2022/0220505 A1
(43) Pub. Date: Jul. 14, 2022

(54) Producer Viruses for Generation of Retroviruses In Situ

(71) Applicant: KaliVir Immunotherapeutics, Inc., Pittsburgh, PA (US)

(72) Inventors: Stephen H. THORNE, Pittsburgh, PA (US); Daniel J. BYRD, Pittsburgh, PA (US)

(21) Appl. No.: 17/703,094

(22) Filed: Mar. 24, 2022

Related U.S. Application Data
(63) Continuation of application No. PCT/US2020/056130, filed on Oct. 16, 2020.
(60) Provisional application No. 62/916,095, filed on Oct. 16, 2019.

(51) Int. Cl. C12N 15/86; C12N 9/12

(52) U.S. Cl. CPC C12N 15/86; C12N 9/1247; C12Y 207/07052; C12N 2710/24121; C12N 2795/10043; C12N 2740/15043; C12N 2740/16043; C12N 2740/13043; C12N 2710/24144

(57) ABSTRACT

This disclosure provides compositions comprising a modified oncolytic virus that can contain modifications in the viral genome and exogenous nucleic acids coding for proteins. The viral compositions and methods provided herein can be utilized for the treatment of cancer.

### CROSS-REFERENCE

This application is a continuation of PCT/US2020/056130, filed Oct. 16, 2020, which claims the benefit of U.S. Provisional Application No. 62/916,095 filed Oct. 16, 2019, which is incorporated by reference herein in its entirety.

### INCORPORATION BY REFERENCE

All publications, patents, patent applications, and NCBI accession numbers mentioned in this specification are herein incorporated by reference to the same extent as if each individual publication, patent, or patent application was specifically and individually indicated to be incorporated by reference, and as if set forth in their entireties. In the event of a conflict between a term as used herein and the term as defined in the incorporated reference, the definition of this disclosure controls.

### SUMMARY

One embodiment provides a virus that produces from its genome a non-replicating retrovirus, wherein the virus is an oncolytic virus. In some embodiments, wherein the non-replicating retrovirus comprises a transgene that codes for a non-retroviral protein. One embodiment provides a virus that produces from its genome a non-replicating retrovirus, wherein the virus is an oncolytic virus, wherein the non-replicating retrovirus comprises a transgene that codes for a non-retroviral protein, and wherein the non-replicating retrovirus is capable of expressing the non-retroviral protein in cells that are infected by the oncolytic virus and in cells that are not infected by the oncolytic virus, in a tumor microenvironment.

One embodiments an oncolytic virus that produces from its genome a non-replicating retrovirus, wherein the non-replicating retrovirus comprises a transgene that codes for a non-lentiviral protein, and wherein the non-replicating retrovirus is capable of infecting and transducing both dividing and non-dividing cells in a target population of mammalian cells. One embodiment provides a virus comprising a transgene that codes for a non-retroviral protein, wherein the transgene is within an exogenous nucleic acid construct that further comprises a gene that codes for a protein of a non-replicating retrovirus, and wherein the virus is oncolytic. One embodiment provides a virus comprising a retroviral envelope construct, a retroviral packing construct, and a retroviral transfer construct, wherein at least one of the envelope construct, the packaging construct, or the transfer construct comprises a transgene that codes for a non-retroviral protein, and wherein the virus is an oncolytic virus.

In some embodiments, wherein the envelope construct, the packing construct, and the transfer construct are inserted at different locations within the genome of the oncolytic virus. In some embodiments, the envelope construct comprises a gene that codes for a retroviral envelope protein and the packaging construct comprises a gene that codes for a retroviral structural protein. In some embodiments, the gene that codes for the retroviral envelope protein is under the control of a first early viral promoter and the gene that codes for the retroviral structural protein is under the control of a second early viral promoter. In some embodiments, the gene that codes for the retroviral envelope protein is under the control of a first early viral promoter, a first early/late viral promoter or a first late viral promoter; and the gene that codes for a retroviral structural protein is under the control of a second early viral promoter, a second early/late viral promoter, or a second late viral promoter. In some embodiments, the envelope construct further comprises a termination sequence at its 5′-terminal region. In some embodiments, the packaging construct further comprises a termination sequence at its 3′-terminal region. In some embodiments, the transfer construct comprises a 3′-long terminal repeat region. In some embodiments, the transfer construct comprises a hybrid 5′-long terminal repeat region. In some embodiments, the hybrid 5′-long terminal repeat region comprises a third early viral promoter and a HIV-1 protein 5′-long terminal repeat region. In some embodiments, the hybrid 5′-long terminal repeat region comprises a HIV-1 protein 5′-long terminal repeat region and a viral promoter, wherein the viral promoter is a third early viral promoter, a third early/late viral promoter, or a third late viral promoter. In some embodiments, the transfer construct further comprises a gene that codes for a self-cleaving RNA ribozyme. In some embodiments, the gene that codes for the self-cleaving RNA ribozyme is positioned downstream to the 3′-long terminal repeat region of the transfer construct. In some embodiments, the self-cleaving ribozyme comprises a delta virus ribozyme or a hammerhead ribozyme. In some embodiments, the packaging construct comprises a 5′-long terminal repeat region and wherein the second early viral promoter is fused to the 5′-long terminal repeat region.

In some embodiments, the envelope construct comprises a termination sequence at its 5′-terminal region. In some embodiments, the packaging construct comprises a termination sequence at its 3′-terminal region. In some embodiments, the transfer construct comprises a termination sequence at its 3′-terminal region. In some embodiments, the termination sequence comprises a nucleotide sequence as set forth in SEQ ID No. 7. In some embodiments, the retroviral structural protein comprises a mature gag-pol protein. In some embodiments, the mature gag-pol protein comprises p55, p41, and p24. In some embodiments, the retroviral envelope protein comprises a VSV-G protein. In some embodiments, the virus comprises an exogenous nucleic acid sequence that codes for a nucleic acid polymerase. In some embodiments, the nucleic acid polymerase is selected from the group consisting of: T7 RNA polymerase, T3 RNA polymerase and SP6 RNA polymerase, a RNA polymerase variant, and a DNA polymerase mutant. In some embodiments, the virus comprises an exogenous nucleic acid sequence that codes for a bacteriophage polymerase. In some embodiments, the bacteriophage is selected from the group consisting of a T3 bacteriophage, a T7 bacteriophage and an SP6 bacteriophage. In some embodiments, the T3 bacteriophage polymerase is expressed with a T3 bacteriophage promoter, a T7 bacteriophage polymerase is expressed with a T7 bacteriophage promoter, and an SP6 bacteriophage polymerase is expressed with an SP6 bacteriophage promoter. In some embodiments, the virus comprises an exogenous nucleic acid sequence that codes for a nucleic acid polymerase. In some embodiments, the nucleic acid polymerase is selected from the group consisting of: T7 RNA polymerase, T3 RNA polymerase and SP6 RNA polymerase, a RNA polymerase variant, and a DNA polymerase mutant. In some embodiments, the virus comprises an exogenous nucleic acid sequence that codes for a bacteriophage polymerase. In some embodiments, the bacteriophage is selected from the group consisting of a T3 bacteriophage, a T7 bacteriophage and an SP6 bacteriophage. In some embodiments, the T3 bacteriophage polymerase is expressed with a T3 bacteriophage promoter, a T7 bacteriophage polymerase is expressed with a T7 bacteriophage promoter, and an SP6 bacteriophage polymerase is expressed with an SP6 bacteriophage promoter.

One embodiment provides a virus comprising: (i) a retroviral envelope construct inserted at a first location within the genome of the virus; and (ii) a retroviral packaging construct inserted at a second location within the genome of the virus, wherein the first location and the second location are not contiguous, wherein the envelope construct comprises a nucleic acid that codes for a retroviral envelope protein, wherein the packaging construct comprises a nucleic acid that codes for a retroviral structural protein, and wherein the virus is an oncolytic virus. In some embodiments, the virus comprises (iii) a transfer construct inserted at a third location within the genome of the oncolytic virus. In some embodiments, the transfer construct comprises a transgene that codes for a non-retroviral protein. In some embodiments, the transfer construct is inserted near the 3′ end of the genome of the oncolytic virus. In some embodiments, the gene that codes for the retroviral envelope protein is under the control of a first early viral promoter and the gene that codes for the retroviral structural protein is under the control of a second early viral promoter. In some embodiments, the gene that codes for the retroviral envelope protein is under the control of a first early viral promoter, a first early/late viral promoter or a first late viral promoter; and the gene that codes for a retroviral structural protein is under the control of a second early viral promoter, a second early/late viral promoter, or a second late viral promoter. In some embodiments, the transfer construct comprises a hybrid 5′-long terminal repeat region. In some embodiments, the hybrid 5′-long terminal repeat region comprises a third early viral promoter and a HIV-1 protein 5′-long terminal repeat region. In some embodiments, the hybrid 5′-long terminal repeat region comprises a HIV-1 protein 5′-long terminal repeat region and a viral promoter, wherein the viral promoter is a third early viral promoter, a third early/late viral promoter, or a third late viral promoter. In some embodiments, the transfer construct further comprises a gene that codes for a self-cleaving RNA ribozyme. In some embodiments, the gene that codes for the self-cleaving RNA ribozyme is positioned downstream to the 3′-long terminal repeat region of the transfer construct. In some embodiments, the self-cleaving ribozyme comprises a delta virus ribozyme or a hammerhead ribozyme. In some embodiments, the packaging construct comprises a 5′-long terminal repeat region and wherein the second early viral promoter is fused to the 5′-long terminal repeat region. In some embodiments, the nucleic acid that codes for the retroviral envelope protein comprises a nucleotide sequence as set forth in SEQ ID No. 1, or a nucleotide sequence that is at least about 70% to at least about 99% identical to the sequence set forth as SEQ ID No. 1.

In some embodiments, the nucleic acid that codes for the retroviral structural protein comprises a nucleotide sequence as set forth in SEQ ID No. 2, or a nucleotide sequence that is at least about 70% to at least about 99% identical to the sequence set forth as SEQ ID No. 2. In some embodiments, the transfer construct comprises a 3′-long terminal repeat region. In some embodiments, the envelope construct comprises a termination sequence at its 5′-terminal region. In some embodiments, the packaging construct comprises a termination sequence at its 3′-terminal region. In some embodiments, the transfer construct comprises a termination sequence at its 3′-terminal region. In some embodiments, the termination sequence comprises nucleotide sequence as set forth in SEQ ID No. 5. In some embodiments, the first location and the second location are separated by at least about 10,000 bases. In some embodiments, the first location and the third location are separated by at least about 10,000 bases. In some embodiments, the second location and the third location are separated by at least about 10,000 bases. In some embodiments, the structural protein comprises a mature gag-pol protein. In some embodiments, the mature gag-pol protein comprises p55, p41, and p24. In some embodiments, the envelope protein comprises a VSV-G protein. In some embodiments, the oncolytic virus is tumor selective in replication. In some embodiments, the non-replicating retrovirus is produced selectively within a tumor microenvironment. In some embodiments, the oncolytic virus in an oncolytic vaccinia virus. In some embodiments, the first, the second, and the third early viral promoters, wherein the first early viral promoter is a first vaccinia virus early promoter, the second early viral promoter is a second vaccinia virus early promoter, and the third early viral promoter is a third vaccinia virus early promoter. In some embodiments, the first vaccinia virus early promoter comprises a sequence as set forth in SEQ ID No. 9. In some embodiments, the second vaccinia virus early promoter comprises a sequence as set forth in SEQ ID No. 10. In some embodiments, the third vaccinia virus early promoter comprises a sequence set forth as SEQ ID No. 11. In some embodiments, the transfer construct comprises a human PGK promoter (SEQ ID No. 12). In some embodiments, the envelope construct is inserted between genes vacwr032 and vacwr033 of the oncolytic vaccinia virus. In some embodiments, the packaging construct is inserted between genes vacwr093 and vacwr095 of the oncolytic vaccinia virus. In some embodiments, the genome of the oncolytic vaccinia virus comprises a deletion of the vacwr094 gene, and wherein the packaging construct is inserted into the location of the vacwr094 gene. In some embodiments, the transfer construct is inserted between genes vacwr205 and vacwr206 of the oncolytic vaccinia virus. In some embodiments, the non-retroviral protein comprises a therapeutic protein or a diagnostic protein. In some embodiments, the non-retroviral protein comprises the therapeutic protein, and wherein the therapeutic protein comprises an immune checkpoint modulator, an antibody or portion thereof, a Fc fusion protein, an anticoagulant, a blood factor, a bone morphogenetic protein, an immunosuppressive agent, an immunostimulatory agent, an enzyme, a growth factor, a hormone, an interferon, an interleukin, a thrombolytic, an anti-angiogenic, a chemotherapeutic, an antibiotic, an antifungal, an antiviral, and any combination thereof.

In some embodiments, the retrovirus is an Alpharetrovirus, a Betaretrovirus, a Deltaretrovirus, an Epsilonretrovirus, a Gamma retrovirus, or a Lentivirus. In some embodiments, the retrovirus is the lentivirus. In some embodiments, the lentivirus is an HIV. In some embodiments, the retrovirus is the Gamma retrovirus. In some embodiments, the Gamma retrovirus is a Moloney murine leukemia virus. In some embodiments, the virus comprises an exogenous nucleic acid sequence that codes for a nucleic acid polymerase. In some embodiments, the nucleic acid polymerase is selected from the group consisting of: T7 RNA polymerase, T3 RNA polymerase and SP6 RNA polymerase, a RNA polymerase variant, and a DNA polymerase mutant. In some embodiments, the virus comprises an exogenous nucleic acid sequence that codes for a bacteriophage polymerase. In some embodiments, the bacteriophage is selected from the group consisting of a T3 bacteriophage, a T7 bacteriophage and an SP6 bacteriophage. In some embodiments, the T3 bacteriophage polymerase is expressed with a T3 bacteriophage promoter, a T7 bacteriophage polymerase is expressed with a T7 bacteriophage promoter, and an SP6 bacteriophage polymerase is expressed with an SP6 bacteriophage promoter.

One embodiment a method of treating cancer, the method comprising: administering to a subject an oncolytic virus according to this disclosure, wherein the non-replicating retrovirus generated from the oncolytic virus is selectively targeted to a tumor tissue. In some embodiments, the tumor tissue comprises a malignant neoplastic tissue. One embodiment provides an engineered producer virus that generates a non-replicating retrovirus from its genome, wherein the engineered producer virus comprises at least one of the following modifications:
i. mutation or deletion of at least one viral gene;
ii. insertion of at least one exogenous nucleic acid;
iii. altered tropism; or
iv. any combinations thereof,
   and wherein the non-replicating retrovirus comprises a transgene that codes for a non-retroviral protein. In some embodiments, the non-replicating lentivirus comprises a transgene that codes for a non-retroviral protein. In some embodiments, the producer virus is a vaccinia virus and the at least one viral gene is selected from the group consisting of: B5R, A52R (VACWR178), F13L, A36R, A34R, A33R, B8R, B18R, SPI-1, SPI-2, B15R, VGF, E3L, K3L, A41L, K7R, NIL, C12L, TK, and any combinations thereof. In some embodiments, the at least one exogenous nucleic acid codes for a protein selected from the group consisting of: CXCR4, CCR2, PH-20, HMGB1, PIAS3, IL15, IL15-Rα, LIGHT, ITAC, fractalkine, CCL5, a metabolic modulating protein, a cytokine, a fusion protein comprising any combinations of the above, and a functional domain or fragment or variant thereof, or any combinations thereof. In some embodiments, the at least one exogenous nucleic acid codes for the PIAS3.

In some embodiments, the producer virus comprises a retroviral envelope construct, a retroviral packaging construct and a retroviral transfer construct, wherein the envelope construct, the packing construct, and the transfer construct are inserted at different locations within the genome of the producer virus. In some embodiments, the envelope construct comprises a gene that codes for a retroviral envelope protein and the packaging construct comprises a gene that codes for a retroviral structural protein. In some embodiments, the gene that codes for the retroviral envelope protein is under the control of a first early viral promoter and the gene that codes for the retroviral structural protein is under the control of a second early viral promoter. In some embodiments, the envelope construct further comprises a termination sequence at its 5′-terminal region.

In some embodiments, the packaging construct further comprises a termination sequence at its 3′-terminal region. In some embodiments, the transfer construct comprises a termination sequence. In some embodiments, the transfer construct comprises a hybrid 5′-long terminal repeat region. In some embodiments, the hybrid 5′-long terminal repeat region comprises a third early viral promoter and a HIV-1 protein 5′-long terminal repeat region. In some embodiments, the packaging construct comprises a 5′-long terminal repeat region and wherein the second early viral promoter is fused to the 5′-long terminal repeat region. In some embodiments, the envelope construct comprises a termination sequence at its 5′-terminal region. In some embodiments, the packaging construct comprises a termination sequence at its 3′-terminal region. In some embodiments, the transfer construct comprises a termination sequence. In some embodiments, the termination sequence comprises a nucleotide sequence as set forth in SEQ ID No. 7. In some embodiments, the retroviral structural protein comprises a mature gag-pol protein. In some embodiments, the mature gag-pol protein comprises p55, p41, and p24. In some embodiments, the retroviral envelope protein comprises a VSV-G protein. In some embodiments, the producer virus in an oncolytic vaccinia virus.

In some embodiments, the envelope construct comprises a first early vaccinia promoter, the packaging construct comprises a second early vaccinia promoter, and the transfer construct comprises a third early vaccinia promoter. In some embodiments, the first vaccinia virus early promoter comprises a sequence as set forth in SEQ ID No. 9. In some embodiments, the second vaccinia virus early promoter comprises a sequence as set forth in SEQ ID No. 10. In some embodiments, the third vaccinia virus early promoter comprises a sequence set forth as SEQ ID No. 11. In some embodiments, the transfer construct comprises a human PGK promoter (SEQ ID No. 12). In some embodiments, the envelope construct is inserted between genes vacwr032 and vacwr033 of the oncolytic vaccinia virus. In some embodiments, the packaging construct is inserted between genes vacwr093 and vacwr095 of the oncolytic vaccinia virus. In some embodiments, the genome of the oncolytic vaccinia virus comprises a deletion of the vacwr094 gene, and wherein the packaging construct is inserted into the location of the vacwr094 gene. In some embodiments, the transfer construct is inserted between genes vacwr205 and vacwr206 of the oncolytic vaccinia virus. In some embodiments, the non-retroviral protein comprises a therapeutic protein or a diagnostic protein. In some embodiments, the virus comprises the transgene that codes for the non-retroviral protein, wherein the non-retroviral protein comprises the therapeutic protein, and wherein the therapeutic protein comprises an immune checkpoint modulator, an antibody or portion thereof, a Fc fusion protein, an anticoagulant, a blood factor, a bone morphogenetic protein, an immunosuppressive agent, an immunostimulatory agent, an enzyme, a growth factor, a hormone, an interferon, an interleukin, a thrombolytic, an anti-angiogenic, a chemotherapeutic, an antibiotic, an antifungal, an antiviral, and any combination thereof. In some embodiments, the retrovirus is an Alpharetrovirus, a Betaretrovirus, a Deltaretrovirus, an Epsilonretrovirus, a Gamma retrovirus, or a Lentivirus. In some embodiments, the retrovirus is a lentivirus. In some embodiments, the lentivirus is an HIV. In some embodiments, the retrovirus is the Gamma retrovirus. In some embodiments, the Gamma retrovirus is a Moloney murine leukemia virus (MMLV).

One embodiment provides a method of treating cancer, the method comprising: administering to a subject an engineered producer virus according to this disclosure, wherein the non-replicating retrovirus generated from the engineered producer virus is selectively targeted to a tumor tissue. In some embodiments, the tumor tissue comprises a malignant neoplastic tissue.

One embodiment provides a process for generating an oncolytic virus that produces a non-replicating retrovirus, the process comprising: growing a population of the oncolytic virus in mammalian cells, followed by adding and selecting for, sequentially, a retroviral envelope construct, a retroviral packaging construct, and a retroviral transfer construct, wherein at least one of the envelope construct, the packaging construct, or the transfer construct comprises a transgene that codes for a non-retroviral protein. In some embodiments, the retrovirus is an Alpharetrovirus, a Betaretrovirus, a Deltaretrovirus, an Epsilonretrovirus, a Gamma retrovirus, or a Lentivirus. In some embodiments, the retrovirus is the Gamma retrovirus. In some embodiments, the Gamma retrovirus is a Moloney murine leukemia virus. In some embodiments, the retrovirus is the Lentivirus. In some embodiments, the Lentivirus is HIV.

### BRIEF DESCRIPTION OF THE DRAWINGS

The novel features of the disclosure are set forth with particularity in the appended claims. A better understanding of the features and advantages of this disclosure will be obtained by reference to the following detailed description that sets forth illustrative embodiments, in which the principles of this disclosure are utilized, and the accompanying drawings of which.

FIG. 1 shows locations of insertions or insertion/deletions of each lentiviral construct within the vaccinia virus genome.

FIG. 2 shows a map of the lentiviral env construct.

FIG. 3 shows a map of the lentiviral packaging construct.

FIG. 4 shows a map of the lentiviral transfer construct.

FIG. 5 shows detection of GAG expression by Western blot.

FIG. 6 shows detection of VSV-G expression by Western blot.

FIG. 7 shows a fluorescent microscopy image of HEK293 cells infected with Lenti-Vac virus generating infectious lentivirus, as shown by the presence of TagRFP-T and sfGFP on the cells; indicating vaccinia virus infection and lentivirus transduction respectively.

FIG. 8 shows an exemplary construct containing a T7 RNA polymerase sequence.

### DETAILED DESCRIPTION

While preferred embodiments of this disclosure have been shown and described herein, it will be obvious to those skilled in the art that such embodiments are provided by way of example only. Numerous variations, changes, and substitutions will now occur to those skilled in the art without departing from this disclosure. It should be understood that various alternatives to the embodiments of this disclosure described herein may be employed in practicing the disclosure. It is intended that the following claims define the scope of the disclosure and that methods and structures within the scope of these claims and their equivalents be covered thereby.

### Certain Definitions

The terminology used herein is for the purpose of describing particular cases only and is not intended to be limiting. As used herein, the singular forms “a”, “an” and “the” can include the plural forms as well, unless the context clearly indicates otherwise. Furthermore, to the extent that the terms “contains,” “containing,” “including”, “includes,” “having,” “has”, “with”, or variants thereof are used in either the detailed description and/or the claims, such terms are intended to be inclusive in a manner similar to the term “comprising.”

The term “about” or “approximately” can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value, such as ±10% of the value modified by the term “about”.

The terms “individual,” “patient,” or “subject” can be used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker). In some embodiments, patients, subjects, or individuals can be under the supervision of a health care worker.

The terms “heterologous nucleic acid sequence,” or “exogenous nucleic acid sequence,” or “transgenes,” as used herein, in relation to a specific virus can refer to a nucleic acid sequence that originates from a source other than the specified virus.

The term “mutation,” as used herein, can refer to a deletion, an insertion of a heterologous nucleic acid, an inversion or a substitution, including an open reading frame ablating mutations as commonly understood in the art.

The term “cancer” and its grammatical equivalents as used herein can refer to a hyperproliferation of cells whose unique trait—loss of normal controls—results in unregulated growth, lack of differentiation, local tissue invasion, and metastasis. With respect to the inventive methods, the cancer can be any cancer, including any of acute lymphocytic cancer, acute myeloid leukemia, alveolar rhabdomyosarcoma, bladder cancer, bone cancer, brain cancer, breast cancer, cancer of the anus, anal canal, rectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, ureter cancer, and/or urinary bladder cancer. As used herein, the term “tumor” refers to an abnormal growth of cells or tissues, e.g., of malignant type or benign type.

The term “gene,” as used herein, can refer to a segment of nucleic acid that encodes an individual protein or RNA (also referred to as a “coding sequence” or “coding region”), optionally together with associated regulatory regions such as promoters, operators, terminators and the like, which may be located upstream or downstream of the coding sequence.

The terms “mutant virus” and “modified virus,” as used interchangeably herein, can refer to a virus comprising one or more mutations in its genome, including but not limited to deletions, insertions of heterologous nucleic acids, inversions, substitutions or combinations thereof.

The term “naturally-occurring,” as used herein with reference to a virus, can indicate that the virus can be found in nature, i.e., it can be isolated from a source in nature and has not been intentionally modified.

The terms “inhibiting,” “reducing” or “prevention,” or any variation of these terms, referred to herein, can include any measurable decrease or complete inhibition to achieve a desired result.

A “promoter,” as used herein, can be a control sequence that is a region of a nucleic acid sequence at which initiation and rate of transcription are controlled. In certain embodiments, a promoter may contain genetic elements at which regulatory proteins and molecules may bind such as RNA polymerase and other transcription factors. The terms “operatively positioned,” “operatively linked,” “under control” and “under transcriptional control” can mean that a promoter is in a correct functional location and/or orientation in relation to a nucleic acid sequence to control transcriptional initiation and/or expression of that sequence. In certain embodiments, a promoter may or may not be used in conjunction with an “enhancer,” which refers to a cis-acting regulatory sequence involved in the transcriptional activation of a nucleic acid sequence.

The term “homology,” as used herein, may be to calculations of “homology” or “percent homology” between two or more nucleotide or amino acid sequences that can be determined by aligning the sequences for optimal comparison purposes (e.g., gaps can be introduced in the sequence of a first sequence). The nucleotides at corresponding positions may then be compared, and the percent identity between the two sequences may be a function of the number of identical positions shared by the sequences (i.e., % homology=# of identical positions/total # of positions×100). For example, a position in the first sequence may be occupied by the same nucleotide as the corresponding position in the second sequence, then the molecules are identical at that position. The percent homology between the two sequences may be a function of the number of identical positions shared by the sequences, taking into account the number of gaps, and the length of each gap, which need to be introduced for optimal alignment of the two sequences. In some embodiments, the length of a sequence aligned for comparison purposes may be at least about: 30%, 40%, 50%, 60%, 65%, 70%, 75%, 80%, 85%, 90%, 91%, 92%, 93%, 94%, 95%, 96%, 97%, 98%, or 95%, of the length of the reference sequence. A BLAST® search may determine homology between two sequences. The homology can be between the entire lengths of two sequences or between fractions of the entire lengths of two sequences. The two sequences can be genes, nucleotides sequences, protein sequences, peptide sequences, amino acid sequences, or fragments thereof. The actual comparison of the two sequences can be accomplished by well-known methods, for example, using a mathematical algorithm. A non-limiting example of such a mathematical algorithm may be described in Karlin, S. and Altschul, S., Proc. Natl. Acad. Sci. USA, 90-5873-5877 (1993). Such an algorithm may be incorporated into the NBLAST and XBLAST programs (version 2.0), as described in Altschul, S. et al., Nucleic Acids Res., 25:3389-3402 (1997). When utilizing BLAST and Gapped BLAST programs, any relevant parameters of the respective programs (e.g., NBLAST) can be used. For example, parameters for sequence comparison can be set at score=100, word length=12, or can be varied (e.g., W=5 or W=20). Other examples include the algorithm of Myers and Miller, CABIOS (1989), ADVANCE, ADAM, BLAT, and FASTA. In another embodiment, the percent identity between two amino acid sequences can be accomplished using, for example, the GAP program in the GCG software package (Accelrys, Cambridge, UK).

The term “subject” can refer to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

The terms “treat,” “treating,” and “treatment” can be meant to include alleviating or abrogating a disorder, disease, or condition; or one or more of the symptoms associated with the disorder, disease, or condition; or alleviating or eradicating the cause(s) of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “therapeutically effective amount” can refer to the amount of a compound that, when administered, can be sufficient to prevent development of, or alleviate to some extent, one or more of the symptoms of the disorder, disease, or condition being treated. The term “therapeutically effective amount” can also refer to the amount of a compound that is sufficient to elicit the biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” can refer to a pharmaceutically-acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The term “pharmaceutical composition” can refer to a mixture of a compound disclosed herein with other chemical components, such as diluents or carriers. The pharmaceutical composition can facilitate administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

An “anti-cancer agent,” as used herein, can refer to an agent or therapy that is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Non-limiting examples of anti-cancer agents can include biological agents (biotherapy), chemotherapy agents, and radiotherapy agents.

The term “oncolytic,” as used herein, can refer to killing of cancer or tumor cells by an agent, such as an oncolytic pox virus, such as an oncolytic vaccinia virus, e.g., through the direct lysis of said cells, by stimulating immune response towards said cells, apoptosis, expression of toxic proteins, autophagy and shut-down of protein synthesis, induction of anti-tumoral immunity, or any combinations thereof. The direct lysis of the cancer or tumor cells infected by the agent, such as an oncolytic vaccinia virus, can be a result of replication of the virus within said cells. In certain examples, the term “oncolytic,” can refer to killing of cancer or tumor cells without lysis of said cells.

The term “oncolytic virus” as used herein can refer to a virus that preferentially infects and kills tumor cells. Under certain non-limiting circumstances, it is understood that oncolytic viruses can promote anti-tumor responses through dual mechanisms dependent on not only the selective killing of tumor cells, but also the stimulation of host anti-tumor immune responses. In some embodiments, the oncolytic viruses can include, but are not limited to, (i) viruses that naturally replicate preferentially in cancer cells and are non-pathogenic in humans often due to elevated sensitivity to innate antiviral signaling or dependence on oncogenic signaling pathways; and (ii) viruses that are genetically-manipulated for use. In some embodiments, the oncolytic virus can be a measles virus, a poliovirus, a poxvirus, a vaccinia virus, an adenovirus, an adeno associated virus, a herpes simplex virus, a vesicular stomatitis virus, a reovirus, a Newcastle disease virus, a senecavirus, a lentivirus, a mengovirus, or a myxomavir. In certain embodiments, the oncolytic virus can be a pox virus. In certain embodiments, the oncolytic virus can be a vaccinia virus.

The term “modified oncolytic virus” as used herein can refer to an oncolytic virus that comprises a modification to its constituent, such as, but not limited to, a modification in the native genome (“backbone”) of the virus like a mutation or a deletion of a viral gene, introduction of an exogenous nucleic acid, a chemical modification of a viral nucleic acid or a viral protein, and introduction of a exogenous protein or modified viral protein to the viral capsid. In general, oncolytic viruses may be modified (also known as “engineered”) in order to gain improved therapeutic effects against tumor cells. In certain embodiments, the modified oncolytic virus can be a modified pox virus. In certain embodiments, the modified oncolytic virus can be a modified pox virus.

The terms “systemic delivery,” and “systemic administration,” used interchangeably herein, in some cases can refer to a route of administration of medication, oncolytic virus or other substances into the circulatory system. The systemic administration may comprise oral administration, parenteral administration, intranasal administration, sublingual administration, rectal administration, transdermal administration, or any combinations thereof.

As used herein the terms “replication deficient virus” or “non-replicating virus,” or “replication incompetent virus,” have their ordinary meaning, such as, a virus that is propagation incompetent as a result of modifications to its genome. Thus, once such recombinant virus infects a cell, the only course it can follow is to express any viral and heterologous protein contained in its genome. In a specific embodiment, the replication defective vectors provided herein may contain genes encoding nonstructural proteins and are self-sufficient for RNA transcription and gene expression. However, these vectors lack genes encoding structural proteins, so that a helper genome is needed to allow them to be packaged into infectious particles. In addition to providing therapeutically safe vectors, the removal of the structural proteins increases the capacity of these vectors to incorporate more than 6 kb of heterologous sequences. In another embodiment, propagation incompetence of the adenovirus vectors of the invention is achieved indirectly, e.g., by removing the packaging signal which allows the structural proteins to be packaged in virions being released from the packaging cell line.

In an aspect, provided herein is a producer virus that can deliver a cargo that comprises a replication defective virus, for instance, a replication defective virus of a different family than the producer virus. In an aspect, the producer virus can be an oncolytic virus, for example a vaccinia virus that produces from its genome a non-replicating virus, such as a non-replicating lentivirus. In some cases, a cargo can be at least a portion of a non-replicating lentivirus (LV). In some cases, a non-replicating lentivirus can comprise a transgene such as a non-lentiviral protein. A non-lentiviral protein can be a therapeutic protein or functional fragment thereof and/or a diagnostic protein. In some cases, the non-replicating lentivirus is capable of expressing the non-lentiviral protein in cells that are infected by the oncolytic virus leading to lysis of the infected cell, such as a cancer cell. In some cases, the non-replicating virus can be produced by the producer virus in situ, such as within a tumor cell or in a tumor micro-environment. For example, the non-replicating virus can be produced in a tumor cell that is infected by the oncolytic producer virus, and the non-replicating virus can express a therapeutic or a diagnostic protein in situ.

In some aspects, provided herein can be a vector system comprising a producer oncolytic virus. In some aspects, a vector system can comprise a disrupted viral genome. In some aspects, a single vector can comprise at least two viral genomes. In some aspects, a single vector can comprise at least 3 viral genomes. In an aspect, a producer oncolytic vaccinia virus genome can comprise a sequence from a foreign virus, such as an HIV virus. In some aspects, a 3-plasmid system can be provided herein in which the env, packaging, and transfer functions of a virus can be on 3 different plasmids. In some aspects, env, packaging and transfer may be on a single vector comprising at least 1 viral genome.

In some examples, a producer virus can be from a family of any one of: Myoviridae, Podoviridae, Siphoviridae, Alloherpesviridae, Herpesviridae, Malacoherpesviridae, Lipothrixviridae, Rudiviridae, Adenoviridae, Ampullaviridae, Ascoviridae, Asfaviridae, Baculoviridae, Bicaudaviridae, Clavaviridae, Corticoviridae, Fuselloviridae, Globuloviridae, Guttaviridae, Hytrosaviridae, Iridoviridae, Marseilleviridae, Mimiviridae, Nimaviridae, Pandoraviridae, Papillomaviridae, Phycodnaviridae, Plasmaviridae, Polydnaviruses, Polyomaviridae, Poxviridae, Sphaerolipoviridae, or Tectiviridae. In an aspect, provided herein a producer oncolytic virus can be a poxviridae virus, such as a vaccinia virus. In an aspect, a vaccinia virus can encode a virus from a different family. For example, a vaccinia virus genome can comprise a second genome for a virus selected from a virus of any of the following families: Myoviridae, Podoviridae, Retroviridae, Siphoviridae, Alloherpesviridae, Herpesviridae, Malacoherpesviridae, Lipothrixviridae, Rudiviridae, Adenoviridae, Ampullaviridae, Ascoviridae, Asfaviridae, Baculoviridae, Bicaudaviridae, Clavaviridae, Corticoviridae, Fuselloviridae, Globuloviridae, Guttaviridae, Hytrosaviridae, Iridoviridae, Marseilleviridae, Mimiviridae, Nimaviridae, Pandoraviridae, Papillomaviridae, Phycodnaviridae, Plasmaviridae, Polydnaviruses, Polyomaviridae, Sphaerolipoviridae, or Tectiviridae.

In some embodiments, a virus produced in situ can be a retrovirus (including lentiviruses), herpes viruses, alphavirus, adeno-associated viruses, vaccinia virus, papillomavirus, or Epstein Barr virus (EBV). In accordance with the present disclosure there may be employed conventional molecular biology, microbiology, and recombinant DNA techniques within the skill of the art. In certain embodiments, a virus produced by the producer virus of this disclosure is replication defective, that is, it may be unable to replicate autonomously in a target cell. Preferably, a replication defective virus is a minimal virus, such that it retains only the sequences of its genome which are necessary for target cell recognition and encapsidating the viral genome. Replication defective virus may not be infective after introduction into a cell. Use of replication defective viral vectors may allow for administration to cells in a specific, localized area, without concern that the vector can infect other cells. Thus, a specific tissue can be specifically targeted, for example a cancer cell.

In an aspect, a virus produced from a producer virus provided herein can be from the family Retroviridae. In an aspect, a Retroviridae virus can be any one of: an Alpharetrovirus, a Betaretrovirus, a Deltaretrovirus, an Epsilonretrovirus, a Gamma retrovirus (e.g., a Moloney murine leukemia virus), or a Lentivirus. In some embodiments, further specific examples of a retrovirus can be any one of: HIV-1, HIV-2, HTLV-1, HTLV-2, HTLV-3, HTLV-4, equine infectious anemia virus (EIAV) lentivirus, or a combination thereof.

The retroviruses produced by the producer viruses disclosed herein are, in some embodiments, integrating viruses which can infect dividing cells as well as non-dividing cells. The retrovirus genome can have two LTRs, an encapsidation sequence and three coding regions (gag, pol and env). Replication defective non-infectious retroviral vectors can be manipulated to destroy the viral packaging signal but can retain the structural genes required to package the co-introduced virus engineered to contain the heterologous gene and the packaging signals. Thus, in recombinant replication defective retroviral vectors, the gag, pol and env genes may generally be deleted, in whole or in part, and replaced with a heterologous nucleic acid sequence of interest. These vectors can be constructed from different types of retroviruses, such as HIV (human immuno-deficiency virus), MoMuLV (murine Moloney leukaemia virus), MSV (murine Moloney sarcoma virus), HaSV (Harvey sarcoma virus), SNV (spleen necrosis virus), RSV (Rous sarcoma virus), and Friend virus.

In a specific embodiment of the disclosure, replication defective lentiviruses produced from an oncolytic producer virus can be used as agents for the direct delivery and sustained expression of a transgene in several tissue types, including brain, retina, muscle, liver, and blood. This subtype of retroviral vectors can efficiently transduce dividing and nondividing cells in these tissues and maintain long-term expression of the gene of interest (for a review, see, Naldini, Curr. Opin. Biotechnol. 1998, 9:457-63; Zufferey, et al., J. Virol. 1998, 72:9873-80). Lentiviral packaging cell lines are available and known generally in the art (see, e.g., Kafri, et al., J. Virol., 1999, 73: 576-584).

In an aspect, a producer virus or a virus produced in situ can also be an adenovirus. Adenoviruses are eukaryotic DNA viruses that can be modified to efficiently deliver a nucleic acid of the disclosure to a variety of cell types. Various serotypes of adenovirus exist, such as type 2 or type 5 human adenoviruses (Ad 2 or Ad 5) or adenoviruses of animal origin (see PCT Publication No. WO94/26914). Those adenoviruses of animal origin can include adenoviruses of canine, bovine, murine (e.g., Mav1 [Beard et al., Virology, 1990, 75:81]), ovine, porcine, avian, and simian (e.g., SAV) origin. Preferably, the adenovirus of animal origin is a canine adenovirus, more preferably a CAV2 adenovirus (e.g., Manhattan or A26/61 strain [ATCC Accession No. VR-800]). Various replication defective adenovirus and minimum adenovirus vectors have been described (PCT Publications No. WO94/26914, WO95/02697, WO94/28938, WO94/28152, WO94/12649, WO95/02697, WO96/22378). The replication defective recombinant adenoviruses according to the disclosure can be prepared by any technique known to the person skilled in the art (Levrero et al., Gene, 1991, 101:195; EP Publication No. 185 573; Graham, EMBO J., 1984, 3:2917; Graham et al., J. Gen. Virol., 1977, 36:59). Recombinant adenoviruses are recovered and purified using standard molecular biological techniques, which are well known to one of ordinary skill in the art. Adeno-associated virus-based vectors. The adeno-associated viruses (AAV) are DNA viruses of relatively small size which can integrate, in a stable and site-specific manner, into the genome of the cells which they infect. They are able to infect a wide spectrum of cells without inducing any effects on cellular growth, morphology or differentiation, and they do not appear to be involved in human pathologies. The AAV genome has been cloned, sequenced and characterized. The use of vectors derived from the AAVs for transferring genes in vitro and in vivo has been described (see PCT Publications No. WO 91/18088 and WO 93/09239; U.S. Pat. Nos. 4,797,368 and 5,139,941; EP Publication No. 488 528). The replication defective recombinant AAVs according to the disclosure can be prepared by cotransfecting a plasmid containing the nucleic acid sequence of interest flanked by two AAV inverted terminal repeat (ITR) regions, and a plasmid carrying the AAV encapsidation genes (rep and cap genes), into a cell line which is infected with producer virus (e.g., an oncolytic vaccinia virus). The AAV recombinants which are produced can then be purified by standard techniques.

### Oncolytic Virus

Provided herein can be an oncolytic producer virus or portion thereof. An oncolytic virus can be a virus that can infect and lyse a cancer cell. In an aspect, an oncolytic virus may have at least one of the following functions in cancer therapy: (1) directly destroying the tumor cells by viral lysis, (2) serving as a vector for expressing heterologous proteins in the tumor site, and (3) the presentation of autologous tumor antigens to prime/activate the immune system. Oncolytic virus selectivity for cancer cells can occur either during infection or during replication. Tumor-selective viruses can be engineered by altering viral surface proteins that recognize specific cellular receptors, allowing the virus to specifically enter cancer cells. Replication selectivity can be accomplished by modifying the viral genes that are required for efficient replication, so that the virus can only replicate in cells that have disruptions in normal homeostatic pathways, such as tumor-suppressor defects or activation of oncogenic pathways.

In an aspect, an oncolytic virus can be any one of: a vesicular stomatitis virus (VSV), a Newcastle disease virus (NDV), a retrovirus, a reovirus, a measles virus, a Sinbis virus, an influenza virus, a herpes simplex virus, vaccinia virus, and an adenovirus. In some cases, an oncolytic virus can comprise a poxvirus. In some cases the poxvirus can comprises a measles virus, a poliovirus, a poxvirus, a vaccinia virus, an adenovirus, an adeno associated virus, a herpes simplex virus, a vesicular stomatitis virus, a reovirus, a Newcastle disease virus, a senecavirus, a lentivirus, a mengovirus, a myxomavir, a betaentomopoxvirus, a yatapoxvirus, a cervidpoxvirus, a gammaentomopoxvirus, a leporipoxvirus, a suipoxvirus, a molluscipoxvirus, a crocodylidpoxvirus, a alphaentomopoxvirus, a capripoxvirus, a avipoxvirus, or a parapoxvirus. In some cases, the poxvirus can comprise a vaccinia virus.

In one embodiment, the oncolytic producer virus can be a vaccinia virus. Vaccinia virus can be a large, complex enveloped virus having a linear double-stranded DNA genome of about 190 kilobases and encodes approximately 250 genes. Vaccinia is well-known for its role as a vaccine that eradicated smallpox. Vaccinia virus is unique among DNA viruses as it replicates only in the cytoplasm of the host cell. Therefore, the large genome is required to code for various enzymes and proteins needed for viral DNA replication. During replication, vaccinia produces several infectious forms which differ in their outer membranes: the intracellular mature virion (IMV), the intracellular enveloped virion (IEV), the cell-associated enveloped virion (CEV) and the extracellular enveloped virion (EEV). IMV is the most abundant infectious form and is thought to be responsible for spread between hosts. A number of genes in vaccinia can be modified in order to improve its properties as an oncolytic virus.

In some cases, the oncolytic producer virus can be Vaccinia virus western reserve (WR), the genome sequence of which is provided in Table 1. In an aspect, a vaccinia virus genome, such as the genome sequence provided in Table 1, can be disrupted with at least 1, 2, 3, 4, 5, 6, 7, or up to 8 exogenous sequences, e.g., exogenous sequences that code for a replication defective virus produced from the vaccinia virus. In an aspect, a producer oncolytic virus provided herein can comprise percent identity from about 10%, 20%, 30%, 40%, 50%, 60%, 70%, 80%, 90%, 95%, 98%, 99%, or up to about 100% to SEQ ID NO: 1.

TABLE 1
Complete genome of Vaccinia Virus WR

| SEQ | |
|---|---|
| ID | |
| NO: | Sequence from GenBank: AY243312.1 |
| 1 | ATTTAAAATATAATATTAATGTACTAAAACTTATATATTATTAATTTATCTAACTAAAGTTAGTAAATTA |
| | TATATATAATTTTATAATTAATTTAATTTTACTAATTTTATTTAGTGTCTAGAAAAAAATGTGTGACCCA |
| | TGACTGTAGGAAACTCTAGAGTGTAAGAAAGATCGATCGCTTTATAGAGACCATCAGAAAGAGGTTTAAT |
| | ATTTTTGTGAGACCATCGAAGAGAGAAAGAGATAAAACTTTTTTACGACTCCATCAGAAAGAGGTTTAAT |
| | ATTTTTGTGAGACCATCGAAGAGAGAAAGAGATAAAACTTTTTTACGACTCCATCAGAAAGAGGTTTAAT |
| | ATTTTTGTGAGACCATCGAAGGAGAAAGAGATAAAACTTTTTTACGACTCCATCAGAAAGAGGTTTAATA |
| | TTTTTGTGAGACCATCGAAGGAGAAAGAGATAAAACTTTTTTACGACTCCATCAGAAAGAGGTTTAATAT |
| | TTTTGTGAGACCATCGAAGAGAGAAAGAGATAAAACTTTTTTACGACTCCATCAGAAAGAGGTTTAATAT |
| | TTTTGTGAGACCATCGAAGAGAGAAAGAGATAAAACTTTTTTACGACTCCATCAGAAAGAGGTTTAATAT |
| | TTTTGTGAGACCATCGAAGGAGAAAGAGATAAAACTTTTTTACGACTCCATCAGAAAGAGGTTTAATATT |
| | TTTGTGAGACCATCGAAGAGAGAAAGAGATAAAACTTTTTTACGACTCCATCAGAAAGAGGTTTAATATT |
| | TTTGTGAGACCATCGAAGAGAGAAAGAGATAAAACTTTTTTACGACTCCATCAGAAAGAGGTTTAATATT |
| | TTTGTGAGACCATCGAAGAGAGAAAGAGATAAAACTTTTTTACGACTCCATCAGAAAGAGGTTTAATATT |
| | TTTGTGAGACCATCGAAGAGAGAAAGAGATAAAACTTTTTTACGACTCCATCAGAAAGAGGTTTAATATT |
| | TTTGTGAGACCATCGAAGAGAGAAAGAGATAAAACTTTTTTACGACTCCATCAGAAAGAGGTTTAATATT |
| | TTTGTGAGACCATCGAAGAGAGAAAGAGAAAGAGATAGTTGATCTAGATATTTTTCTTAGTACAAAAGTC |
| | AATGTTTTAAAATATATGGACAAGAATTTGTCTGTATAAAAACTTGTGTGAAATTTTGTACCAAAGAAAA |
| | AATGTGAGCAGTATCCCCTACATGGATTTTACTAGATCATTTATATACCAAAAAATATTATACGATCTAC |
| | GTTTTATTATATGATTTTAACGTGTAAATTATAAACATTATTTTATGATATACAATTGTCTGGTAACCTA |
| | GATGGGCATAGGGGATGTTGATAAGCTCGACGAGTATATGTTGTTGGACGTTATTGTTTAAGAAATAGTT |
| | GATGCATCAGAAAGAGAATAAAAAATATTTTAGTGAGACCATCGAAGAGAGAAAGAGATAAAACTTTTTT |
| | ACGACTCCATCAGAAAGAGGTTTAATATTTTTGTGAGACCATCGAAGAGAGAAAGAGATAAAACTTTTTT |
| | ACGACTCCATCAGAAAGAGGTTTAATATTTTTGTGAGACCATCGAAGAGAGAAAGAGATAAAACTTTTTT |
| | ACGACTCCATCAGAAAGAGGTTTAATATTTTTGTGAGACCATCGAAGAGAGAAAGAGATAAAACTTTTTT |
| | ACGACTCCATCAGAAAGAGGTTTAATATTTTTGTGAGACCATCGAAGAGAGAAAGAGATAAAACTTTTTT |
| | ACGACTCCATCAGAAAGAGGTTTAATATTTTTGTGAGACCATCGAAGAGAGAAAGAGATAAAACTTTTTT |
| | ACGACTCCATCAGAAAGAGGTTTAATATTTTTGTGAGACCATCGAAGAGAGAAAGAGATAAAACTTTTTT |
| | ACGACTCCATCAGAAAGAGGTTTAATATTTTTGTGAGACCATCGAAGAGAGAAAGAGATAAAACTTTTTT |
| | ACGACTCCATCAGAAAGAGGTTTAATATTTTTGTGAGACCATCGAAGAGAGAAAGAGATAAAACTTTTTT |
| | ACGACTCCATCAGAAAGAGGTTTAATATTTTTGTGAGACCATCGAAGAGAGAAAGAGATAAAACTTTTTT |
| | ACGACTCCATCAGAAAGAGGTTTAATATTTTTGTGAGACCATCGAAGAGAGAAAGAGATAAAACTTTTTT |
| | ACGACTCCATCAGAAAGAGGTTTAATATTTTTGTGAGACCATCGAAGAGAGAAAGAGATAAAACTTTTTT |
| | ACGACTCCATCAGAAAGAGGTTTAATATTTTTGTGAGACCATCGAAGAGAGAAAGAGATAAAACTTTTTT |
| | ACGACTCCATCAGAAAGAGGTTTAATATTTTTGTGAGACCATCGAAGAGAGAAAGAGATAAAACTTTTTT |
| | ACGACTCCATCAGAAAGAGGTTTAATATTTTTGTGAGACCATCGAAGAGAGAAAGAGATAAAACTTTTTT |
| | ACGACTCCATCAGAAAGAGGTTTAATATTTTTGTGAGACCATCGAAGAGAGAAAGAGATAAAACTTTTTT |
| | ACGACTCCATCAGAAAGAGGTTTAATATTTTTGTGAGACCATCGAAGAGAGAAAGAGATAAAACTTTTTT |
| | ACGACTCCATCAGAAAGAGGTTTAATATTTTTGTGAGACCATCGAAGAGAGAAAGAGATAAAACTTTTTT |
| | ACGACTCCATCAGAAAGAGGTTTAATATTTTTGTGAGACCATCGAAGAGAGAAAGAGAATAAAAATATTT |
| | TATGACTCCATTGAAGAGAGAAAGAGAAAATGAGAATGAGAATAAAAATATTTTAGTGACACCATCAGAA |
| | AGAGGTTTAATATTTTTGTGAGACCATCGAAGAGAGAAAGAGAATAAAAATATTTTATGACTCCATTGAA |
| | GAGAGAAAGAGAAAATGAGAATGAGAATAAAAATATTTTAGTGACACCATCAGAAAGAGGTTTAATATTT |
| | TTTATGAGACCATCAAAGAGAGAAAGAGAATAAAAATATTTTTGTAAAACTTTTTTTATGAGACCATCAA |
| | AGAGAGAAAGAGAATAAAAATATTTTTGTAAAACTTTTTTTATGAGACCATCAAAGAGAGAAAGAGAATA |
| | AAAATATTTTTGTAAAACTTTTTTTATGAGACCATCAAAGAGAGAAAGAGAATAAAAATATTTTTGTAAA |
| | ACTTTTTTTATGAGACCATCAAAGAGAGAAAGAGAATAAAAATATTTTTGTAAAACTTTTTTTATGAGAC |
| | CATCAAAGAGAGAAAGAGAATAAAAATATTTTTGTAAAACTTTTTTTATGAGACCATCAAAGAGAGAAAG |
| | AGAATAAAAATATTTTTGTAAAACTTTTTTTATGAGACCATCAAAGAGAGAAAGAGAATAAAAATATTTT |
| | TGTAAAACTTTTTTTATGAGACCATCAGAAAGAGGTTTAATATTTTTGTGATACCCTGAAAGGAAATAGG |
| | AATAGGAATAGGAATAGTGTCATAATCGTATCACACTATTGAGACAGAAAAAGAAGAAGTCGCGAGAGGT |
| | AACTTTTTGTGAATGTAGTTAAGAACATTTTTGTTTTGCAAACCGGAATATAGTGTCCGGTACACTTTTT |
| | TAATTCGTGGTGTGCCTGAATCGTTCGATTAACCCTACTCATCCAATTTCAGATGAATAGAGTTATCGAT |
| | TCAGACACACGCTTTGAGTTTTGTTGAATCGATGAGTGAAGTATCATCGGTTGCACCTTCAGATGCCGAT |
| | CCGTCGACATACTTAAATCCATCCTTGACCTCAAGTTCAGATGATTCCTTGCACATGTCTCCGATACGAA |
| | CGCTAAACTCTAGATTCTTGACACATTTTGTATCGACGATCGTTGAACCGATGATATCTTCGTAACTCAC |
| | TTTCTTATGAGAGATGTTAGACCCGAGTACTGGATGGGTCTTGATGTCGCTGTCTTTCTCTTCTTCGCTA |
| | CATCTGATGTCGATAGACACCTCACAGTCTTTGATCATAGCCAGAGCTTCTTCATGAGTGATCGCGGGAG |
| | AGTCCTTACCTTGTCCTGGGGACACGCTGGACAATCTAGCATTCACTGTGTTTCCATCAGCGGATTCTGA |
| | GATGGATTTAATCTGAGGACATTTGGTGAATCCAAAGTTCATTCTCAGACCTCCACCGATGATGGAGTAA |
| | TAAGTGGTAGGAGGATCTACATCCTCGACTGATGTGGAATCATCTTCTGATTCCACCTCGGGATCTGGAT |
| | CTGACTCGGACTCTGTAATTTCCGTTACGGATTGGCAAATCTTATCATTGGTCGGTGTTTGGTCTTGCTT |
| | TGTGACTTTGATAATAACATCGATTCCCATATGATGTTTGTTTTCTTCTTCCGTACACGAGGAGGAGGAT |
| | GAGGATGATTGCTGAAGACTGGCAGGCACATGCATGCCAGGACGATATATTGTTTCATGATTGCTATTGA |
| | TTGAGTACTGTTCTTTATGATTCTACTTCCTTACCGTGCAATAAATTAGAATATATTTTCTACTTTTACG |
| | AGAAATTAATTATTGTATTTATTATTTATGGGTGAAAAACTTACTATAAAAAGCGGGTGGGTTTGGAATT |
| | AGTGATCAGTTTATGTATATCGCAACTACCGGGCATATGGCTATCGACATCGAGAACATTACCCACATGA |
| | TAAGAGATTGTATCAGTTTCGTAGTCTTGAGTATTGGTATTACTATATAGTATATAGATGTCGACGCTAG |
| | ATAGACAGTCTCCGAATGCGGCATGATACCGTCATCATTCTTTGCTTTCGTTAACTGTTTGGAGGAAAAA |
| | TTTTTGTTATTGCATTTAATCTCGAAATTCAGAGTGCACACCTTTCTCCTGTAAAGAAACCTGAAGTTGC |
| | TACCTTATTAAGGACGGAGAAGTATTCCTCACGAAATACGGGATTACAGTCTTTATGATTCATAGTAATA |
| | GTTAGTTCCGACGTTGAGATGGATTCGCTGAGACCGGTAGTGGTCGTTAACTGGATACAGATTAATTTCC |
| | ACATCGATATAGTTAAAGGTATTACTGGGTACGGGTTCGCATTTATCTGCGGAAGAGACGGTGTGAGAAT |
| | ATGTTCCGAGACCACACGGAGAACAGATGACGTCTCCGGATACTCCGTATCCTATTCCACATTTTGTTTG |
| | GGAAACACATGCCTTGCATCCGGATGATCCTTTGAGAAGACAATAATATCCGGGAGAGCATTCACAGATT |
| | CTATTGTGAGTCGTGTTACACGGTCGCGTCTTCCGTTACAACTTAGACAAGCGGGTAAATGATTATTGCG |
| | AGATGTGAAGGTACCCGAACCACACGGCGTACATTGTGTGTTAGTCTTGCTATCGCATAATCTGGAAGCG |
| | TATGTTCCCGGACACAAATTATGGCGTTTGTATTCGTTGTCTTTACACTTTCCATCGGATGGTGCATGCG |
| | GTGCTATATCTCTTCCGTTTATTATTATACATGAGAGAAACAATATATACGAGTATAATACGGACTTCAT |
| | GATTTAATAATGTAGTAATCGTCGTCTTGTTCCTGTTTCCTACTTCTCCAATCATATAGATATTTTCTTT |
| | CTATCATGGATAATATTTGTAATGGTTCTTTTCGTACAACATACTGTTTAGATGATATTGCGCATAATTT |
| | CCGGAGGCAAATACGATAGTCTAGATTGACCGATGGTAGACTCTAATTTATTGAGTGCTTTGTCGACGAG |
| | TTTACTTTTACGCTCCATCGATAGATGGCACTGTTCTATGAGATCGTCGTACATGGGAAATGAAATGTGA |
| | CTGTCTGAATGTATGGCTTTAAGATAGCTGTGATACCGTATACAGGTCGGTGTCGGAGATTCGAATCTCT |
| | TTAAGGCGACTTATGTCACGATGATGGAATCTATCTTATCGAATGATATATTTTTCATAAATACACTTTT |
| | ATAGTCCTCGTTTAAACAGAATTTACTATGTAGTTCCGCGAATGACTCGTCCCTTAATAGGCAGTAGGCT |
| | ATTATCTTCTTTACGTAGTAATCGTCGTAGGGAGAGACATCTTGTAGAACAACGATTTAATCATAGGTAG |
| | AGATACTTTCAGTCTGTGGTGGATGATGTCATTCACAACATCCGCCTTGTATATGATGTTTCTGTTTTCA |
| | AACACCAAGTCGAATACCGTCTTTAGTCGGAAGGTTGATGTCGTATCCGATGTATGAGGCAACATTGTTG |
| | TTACAATTTTGAAAGGCGGTATTATAGTATTCGTCTTTCTGAATGTCGAACCTATCTAGTAGATACCGTA |
| | GTATATTGAGAGTGTATCCTTGATTATGTTTTATGAATAGATAAAGTAGATGTTGTCCTTCTTCCTTTTG |
| | TTCGTGCCAATTGAGTAACATTATGAGAATATGACCTGTTGCACAATCGTTCCATGATGGGTGTACAATC |
| | AAGATTATTACGTATCCTCGTATCGGCTCCTCGAGATAAAAGAGCATACACCACACGAGGACTATGTTTG |
| | GTATACTGTTGAAGGTAAGTGTGTAACCGCGTTAATGTTTGCTCCATAATCTATTATCGCGTAGATGAAT |
| | CGCTTCTCGGCTCGCATCTTAGTGTGACTTAACTTGTAATAATTGCTTTTGTAGAACGTGGATATGTGTT |
| | TACAGTAGTAATGAAGAGAAGTGAGTTCATCCTCGTCGGATCCTTTGTACAGAACGTAATAGTTTAAGCT |
| | CCCATTGAATTTATATCTAAGATAACACAGCAATAGATCGGATGATTTACTAAAGTCATCAATGGTGTCC |
| | GTTAGTATATCAAAGATCTTGTTATCGATTGATAGTGGTGTCCTTTTTCATCCTTGCTATCAAAGTTACG |
| | CATGCCGTGGTGTAACAATATCTTTAATACAGATGGATTAAATCGTGTATTCATCGTATAGCAATGTAAT |
| | GGAGAGTTACCTCGTTTATTCAGATCGCAGTGTTTAATAACTAGCTTAAACAGATGAGACGATGTATCCA |
| | CATCAAAGAACGTGAAATACATATGACAGACATTGTTGACAGAAACGTGACCTTCATTCTTACCGTCGTC |
| | CATAAATACGTTAGGTATGTACCACATACTGTCGCGAACGATGCGTACAATCTCGTCCATCTCATAATGA |
| | TTTACTTTTTCATAATTAAAGATGTGAAAGAAAAACAGAACAATATATTTTTTTAGTAATGTTTATGCGA |
| | GACATATAAAATAAACTCCGTGTTTATGATCATTTTTAACAGCAACACATTCAATATTGTATTGTTATTT |
| | TTATATTATTTACACAATTAACAATATATTATTAGTTTATATTACTGAATTAATAATATAAAATTCCCAA |
| | TCTTGTCATAAACACACACTGAGAAACAGCATAAACACAAAATCCATCAAAAATGTCGATGAAATATCTG |
| | ATGTTGTTGTTCGCTGCTATGATAATCAGATCATTCGCCGATAGTGGTAACGCTATCGAAACGACATCGC |
| | CAGAAATTACAAACGCTACAACAGATATTCCAGCTATCAGATTATGCGGTCCAGAGGGAGATGGATATTG |
| | TTTACACGGTGACTGTATCCACGCTAGAGATATTGACGGTATGTATTGTAGATGCTCTCATGGTTATACA |
| | GGCATTAGATGTCAGCATGTAGTATTAGTAGACTATCAACGTTCAGAAAACCCAAACACTACAACGTCAT |
| | ATATCCCATCTCCCGGTATTATGCTTGTATTAGTAGGCATTATTATTATTACGTGTTGTCTATTATCTGT |
| | TTATAGGTTCACTCGACGAACTAAACTACCTATACAAGATATGGTTGTGCCATAATTTTTATAAATTTTT |
| | TTATGAGTATTTTTACAAAAAAAATGTATAAAGTGTATGTCTTATGTATATTTATAAAAATGCTAAGTAT |
| | GCGATGTATCTATGTTATTTGTATTTATCTAAACAATACCTCTACCTCTAGATATTATACAAAAATTTTT |
| | TATTTCGGCATATTAAAGTAAAATCTAGTTACCTTGAAAATGAATACAGTGGGTGGTTCCGTATCACCAG |
| | TAAGAACATAATAGTCGAATACAGTATCCGATTGAGATTTTGCATACAATACTAGTCTAGAAAGAAATTT |
| | GTAATCATCTTCTGTGACGGGAGTCCATATATCTGTATCATCGTCTAGTTTATCAGTGTCCCATGCTATA |
| | TTCCTGTTATCATCATTAGTTAATGAAAATAACTCTCGTGCTTCAGAAAAGTCAAATATTGTATCCATAC |
| | ATACATCTCCAAAACTATCGCTTATACGTTTATCTTTAACGATACCTATACCTAGATGGTTATTTACTAA |
| | CAGACATTTTCCAGATCTATTGACTATAACTCCTATAGTTTCCACATCAACCAAGTAATGATCATCTATT |
| | GTTATATAACAATAACATAACTCTTTTCCATTTTTATCAGTATGTATATCTATATCAACGTCGTCGTTGT |
| | AGTGAATAGTAGTCATTGATCTATTATATGAAACGGATATGTCTAGAACGGCAATTGTTTTACGTCCAGT |
| | TAACACTTTCTTTGATTTAAAGTCTAGAGTCTTTGCAAACATAATATCCTTATCCGACTTTATATTTCCT |
| | GTAGGGTGGTATAATTTTATTTTGCCTCCACATATCGGTGTTTCCAAATATATTACTAGACAATATTCCA |
| | TATAGTTATTAGTTAAGGGTACCCAATTAGAACACGTACGCTTATTATCATCATTTGGATCGTATTTCAT |
| | AAAAGTTATTGTACTATCGATGTCAACACATTCTACATTTTTTAATCGTCTATATAGTATTTTTCTGATA |
| | TTTTCTATAATATCAGAATTGTCTTCCATCGGAAGTTGTATACTATCGGAATCAGTTACATGTTTAAATA |
| | ATTCTCTGATGTCATTCCTTATACAATCAAATTCATTATTAAACAGTTTAATAGTCTGTAGACCTTTATC |
| | GTCGTAAATATCCATTGTCTTATTAGTTACGCTTATTTTTATGTGTTTTACGTTGCTTTATTATATTTTA |
| | TAAGAATGATTGTTTGACGAATCACGAGAACTATTAAGACACATTATTAGGTATATATTATAAAAAAGTT |
| | TTTGATTACGATGTTATAAGAGGAAAGAGGACACATTAACATCATACATCAATTAACTACATTCTTATAA |
| | CATCGTAATCAAAAGAATTGCAATTTTGATGTATAACAACTGTCAATGGGTTATGGAATTGTATATTACA |
| | TATTATACGGTATGTTGGTAACGACAAATACCGATCGGTAATTGTCTGCCGGTGTAATAGAATTATATAT |
| | ATCTATCTATTACACCGGCTGAGTATGCATAATAATAAGTTGTGGTAGTATGATCTCCATATTTATAATT |
| | TAGGACTTTGTATTCAGTATTTTTGGAATCATAAAAAATAAAAAAAAGTTTTACTAATTTAAAATTTAAA |
| | AAGTATTTACATTTTTTTCACTGTTTAGTCGCGGATATGGAATTCGATCCTGCCAAAATCAATACATCAT |
| | CTATAGATCATGTAACAATATTACAATACATAGATGAACCAAATGATATAAGACTAACAGTATGCATTAT |
| | CCGAAATATTAATAACATTACATATTATATCAATATCACAAAAATAAATACACATTTGGCTAATCAATTT |
| | CGGGCTTGGAAAAAACGTATCGCCGGAAGGGACTATATGACTAACTTATCTAGAGATACAGGAATACAAC |
| | AATCAAAACTTACTGAAACTATACGTAACTGTCAAAAAAATAGAAACATATATGGTCTATATATACACTA |
| | CAATTTAGTTATTAATGTGGTTATTGATTGGATAACCGATGTGATTGTTCAATCAATATTAAGAGGGTTG |
| | GTAAATTGGTACATAGCTAATAATACCTATACACCCAATAATACAACAACCATTTCTGAGTTGGATATCA |
| | TCAAAATACTGGATAAATACGAGGACGTGTATAGAGTAAGTAAAGAAAAAGAATGTGGAATTTGCTATGA |
| | AGTTGTTTACTCAAAACGATAGATACTTTGGTTTATTGGATTCGTGTACTCATATATTTTGCATAACATG |
| | CATCAATATATGGCATAAAACACGAAGAGAAACCGGTGCGTCGGATAATTGTCCTATATGTCGTACCCGT |
| | TTTAGAAACATAACAATGAGCAAGTTCTATAAGCTAGTTAACTAATAAATAAAAAGTTTAATTTGTTGAC |
| | GACGTATGTCGTTATTTTTCTCGTATGAAAGATTAAATTCAATTCAATTCGTTGTTTCTAATATAATCTG |
| | CCGTATTGGATGGATTCTCAAGACAATTGCATTTAGATTATATTATCATGAATAAAAATAGTAGCACGCA |
| | CTACTTCAGCCAAATATTCTTTTTTGAAACGCCATCTATCGTAGTGAGGACACAAGTGAACCTATAATTA |
| | TCAAATTTATTAGTATCAGTCACATGAAGGACTTTCTGTAGAGTGACGATTCTACCATCTATGGTACTAA |
| | CGGTTTCATCCTCCTTGATACCCTCACCCAAATGTTCTATAAATTTAGCATCCTCGTCCGATCTCATATC |
| | CTTTGCCAACCAATACATGTAGCTAAAATTAGGCATAAATTTCACACATCCAGTGCAACGAAATTCTCCA |
| | GAAGATGTTACGATGTTTAGGTTAGGACATTTGATTTCGTCGGCATTAACATATGGGTGAACACACCCAT |
| | ACATGAAAGCGATGAGAAATAGGATTCTCATCTTGCCAAAATATCACTAGAAAAAATTTATTTATCAATT |
| | TTAAAGGTATAAAAAATACTTATTGTTGCTCGAATATTTTGTATTTGATGGTATACGGAAGATTAGAAAT |
| | GTAGGTATTATCATCAACTGATTCTATGGTTTTATGTATTCTATCATGTTTCACTATTGCGTCGGAAATA |
| | ATATCATATGCTTCCACATATATTTTATTTTGTTTTAACTCATAATACTCACGTAATTCTGGATTATTGG |
| | CATATCTATGAATAATTTTAGCTCCATGATCAGTAAATATTAATGAGAACATAGTATTACCACCTACCAT |
| | TATTTTTTTCATTTCGTTCAATTCTTGATTGCAAAGATCTATATAATCATTATAGCGTTGACTTATGGAC |
| | TCTGGAATCTTAGACGATGTACAGTCATCTATAATCATGGCATATTTAATACATTGTTTTATAGCATAGT |
| | AGTTATCTACGATGTTAGATATTTCTCTCAATGAATCAATCACACAATCTAATGTAGGTTTATGACATAA |
| | TAGCATTTTCAGCAGTTCAATGTTTCTAGATTCGTTGATGGCAATGGCTATACATGTATATCCGTTATTT |
| | GATCTAATGTTGACATCTGAACCGGATTCTAGCAGTAAAGATACTAGAGATTGTTTATTATATCTAACAG |
| | CCTTGTGAAGAAGTGTTTCTCCTCGTTTGTCAATCATGTTAATGTCTTTAAGATAAGGTAGGCAAATGTT |
| | TATAGTACTAAGAATTGGGCAAGCATAAGACATGTCACAAAGACCCTTTTTGTATGTATAAGTGTAAAAA |
| | TTATAACATTCATAGTTGGATTTACATAGGTGTCCAATCGGGATCTCTCCATCATCGAGATAATTGATGG |
| | CATCTCCCTTCCTTTTTTAGTAGATATTTCATCGTGTAAGAATCAATATTAATATTTCTAAAGTATTCGT |
| | GTATAGCCTCTTTATTTACCACAGTTCCATATTCCACTAGAGGGATATCGCCGAATGTCATATACTCAAT |
| | TAGTATATGTTGGAGGACATCCGAGTTCATTGTTTTCAATATCAAAAAGATGGTTTCCTTATCATTTCTC |
| | CATAGTGGTACAATACTACACATTATTCCGTGCGGCTTTCCATTTTCCAAAAACAATTTGACCAAATCTA |
| | AATCTACATCTTTATTGTATCTATAATCACTATTTAGATAATCAGCCATAATTACTCGAGTGCAACATGT |
| | TAGATCGTCTATATATGAATAAGCAGTGTTATCTATTCCTTTCATTAACAATTTAACGATGTCTATATCT |
| | ATATGAGATGACTTAATATAATATTGAAGAGCTGTACAATAGTTTTTATCTATAGAAGACGGCTTGATTC |
| | CGTGATTAATTAGACATTTAACAACTTCCGGACGCACATATGCTCTCGTATCCGACTTTGAATACAGATG |
| | AGAGATGATATACAGATGCAATACGGTACCGCAATTTCGTAGTTGATAATCATCATACGCGTATCAGTAC |
| | TCGTCCTCATAAAGAACACTGCAGCCATTTTCTATGAACAAATCAATAATTTTAGGAACAGGATCATTGT |
| | CATTACATAATTTTCTATAACTGAACGATGGTTTTCACATTTAACACTCAAGTCAAATCCATGTTCTACC |
| | AACACCTTTATCAAGTCAACGTCTACATTTTTGGATTTCATATAGCTGAATATATTAAAGTCATTTATGT |
| | TGCTAAATCCAGTGGCTTCTAGTAGAGCCATCGCTATATCCTTTAACTTTAACATGTCTACTATTTGTGT |
| | ATTCTTCTAATGGGGTAGCTGTCTCCAATTTTTGCGTAATGGATTAGTGCCACTGTCTAGTAGTAGTTTG |
| | ACGACCTCGACATTATTACAATGCTCATTAAAAAGGTATGCGTGTAAAGCATTATTCTTGAATTGGTTCC |
| | TGGTATCATTAGGATCTCTGTCTCTCAACATCTGTTTAAGTTCATCGAGAGCCACCTCCTCATTTTCCAG |
| | ATAGTCAAACATTTTGACTGAATGAGCTACTGTGAACTCTATACACCCACACAACTAATGTCATTAAATA |
| | TTATTTTTTTGAATGTATTTATACCATGTCAAAAACTTGTACAATTATTAATAAAAATAATTTAGTGTTT |
| | AAATTTTACCAGTTCCAGATTTTACACCTCCGTTAACCCCACTTTTTACACCACTGGACGATCCTCCTCC |
| | CCACATTCCACCGCCACCAGATGTATAAGTTTTAGATCCTTTATTACTACCATCATGTCCATGGATAAAG |
| | ACACTCCACATGCCGCCACTACTACCCCCTTTAGAAGACATATTAATAAGACTTAAGGACAAGTTTAACA |
| | ATAAAATTAATCACGAGTACCCTACTACCAACCTACACTATTATATGATTATAGTTTCTATTTTTACAGT |
| | ACCTTAACTAAAGTCTCTAGTCACAAGAGCAATACTACCAACCTACACTATTATATGATTATAGTTTCTA |
| | TTTTTATAGGAACGCGTACGAGAAAATCAAATGTCTAATTTCTAACGGTAGTGTTGATAAACGATTATCG |
| | TCAATGGATACCTCCTCTATCATGTCGTCTATTTTCTTACTTTGTTCTATTAACTTATTAGCATTATATA |
| | TTATTTGATTATAAAACTTATATTGCTTATTAGCCCAATCTGTAAATATCGGATTATTAACATATCGTTT |
| | CTTTGTAGGTTTATTTAACATGTACATCACTGTAAGCATGTCCGTACCATTTATTTTAATTTGACGCATA |
| | TCCGCAATTTCTTTTTCGCAGTCGGTTATAAATTCTATATATGATGGATACATGCTACATGTGTACTTAT |
| | AATCGACTAATATGAAGTACTTGATACATATTTTCAGTAACGATTTATTATTACCACCTATGAATAAGTA |
| | CCTGTGATCGTCTAGGTAATCAACTGTTTTCTTAATACATTCGATGGTTGGTAATTTACTCAGAATAATT |
| | TCCAATATCTTAATATATAATTCTGCTATTTCTGGGATATATTTATCTGCCAGTATAACACAAATAGTAA |
| | TACATGTAAACCCATATTTTGTTATTATATTAATGTCTGCGCCATTATCTATTAACCATTCTACTAGGCT |
| | GACACTATGCGACTTAATACAATGATAAAGTATACTACATCCATGTTTATCTATTTTGTTTATATCATCA |
| | ATATACGGCTTACAAAGTTTTAGTATCGATAACACATCCAACTCACGCATAGAGAAGGTAGGGAATAATG |
| | GCATAATATTTATTAGGTTATCATCATTGTCATTATCTACAACTAAGTTTCCATTTTTTAAAATATACTC |
| | GACAACTTTAGGATCTCTATTGCCAAATTTTTGAAAATATTTATTTATATGCTTAAATCTATATAATGTA |
| | GCTCCTTCATCAATCATACATTTAATAACATTGATGTATACTGTATGATAAGATACATATTCTAACAATA |
| | GATCTTGTATAGAATCTGTATATCTTTTAAGAATTGTGGATATTAGGATATTATTACGTAAACTATTACA |
| | CAATTCTAAAATATAAAACGTATCACGGTCGAATAATAGTTGATCAACTATATAATTATCGATTTTGTGA |
| | TTTTTCTTCCTAAACTGTTTACGTAAATAGTTAGATAGAATATTCATTAGTTCATGACCACTATAGTTAC |
| | TATCGAATAACGCGTCAAATATTTCCCGTTTAATATCGCATTTGTCAAGATAATAATAGAGTGTGGTATG |
| | TTCACGATAAGTATAATAACGCATCTCTTTTTTGTGTGAAATTAAATAGTTTATCACGTCCAAAGATGTA |
| | GCATAACCATCTTGTGACCTAGTAATAATATAATAATAGAGAACTGTTTTACCCATTCTATCATCATAAT |
| | CAGTGGTGTAGTCGTAATCGTAATCGTCTAATTCATCATCCCAATTATAATATTCACCAGCACGTCTAAT |
| | CTGTTCTATTTTGATCTTGTATCCATACTGTATGTTGCTACATGTAGGTATTCCTTTATCCAATAATAGT |
| | TTAAACACATCTACATTGGGATTTGATGTTGTAGCGTATTTCTCTACAATATTAATACCATTTTTGATAC |
| | TATTTATTTCTATACCTTTCGAAATTAGTAATTTCAATAAGTCTATATCGATGTTATCAGAACATAGATA |
| | TTCGAATATATCAAAATCATTGATATTTTTATAGTCGACTGACGACAATAACAAAATCACAACATCGTTT |
| | TTGATATTATTATTTTTCTTGGTAACGTATGCCTTTAATGGAGTTTCACCATCATACTCATATAATGGAT |
| | TTGCACCACTTTCTATCAATGATTGTGCACTGCTGGCATCGATGTTAAATGTTTTACAACTATCATAGAG |
| | TATCTTATCGTTAACCATGATTGGTTGTTGATGCTATCGCATTTTTTGGTTTCTTTCATTTCAGTTATGT |
| | ATGGATTTAGCACGTTTGGGAAGCATGAGCTCATATGATTTCAGTACTGTAGTGTCAGTACTATTAGTTT |
| | CGATCAGATCAATGTCTAGATCTATAGAATCAAAACACGATAGGTCAGAAGATAATGAATATCTGTACGC |
| | TTCTTTTTGTACTGTAACTTCTGGTTTTGTTAGATGGTTGCATCGTGCTTTAACATCAATGGTACAAATT |
| | TTATCCTCGCTTTGTGTATCATATTCGTCTCTAGTATAAAATTCTATATTCAGATTATCATGCGATGTGT |
| | ATACGCTAACGGTATCAATAAACGGAGCACACCATTTAGTCATAACAGTAATCCAAAATTTTTTAAAGTA |
| | TATCTTAACGAAAGAAGTTGTGTCATTGTCTACGGTGTATGGTACTAGATCCTCATAAGTGTATATATCT |
| | AGAGTAATGTTTAATTTATTAAATGGTTGATAATATGGATCCTCATGACAATTTCCGAAGATGGAAATGA |
| | GATATAGACATGCAATAAATCTAATCGAAGACATGGTTACTCCTTAAAAAAATACGAATAATCACCTTGG |
| | CTATTTAGTAAGTGTCATTTAACACTATACTCATATTAATCCATGGACTCATAATCTCTATACGGGATTA |
| | ACGGATGTTCTATATACGGGGATGAGTAGTTTTCTTCTTTAACTTTATACTTTTTACTAATCATATTTAG |
| | ACTGATGTATGGGTAATAGTGTTTAAAGAGTTCGTTCTCATCATCAGAATAAATCAATATCTCTGTTTTT |
| | TTGTTATACAGATGTATTACAGCCTCATATATTACGTAATAGAACGTGTCATCTACCTTATTAACTTTCA |
| | CCGCATAGTTGTTTGCAAATACGGTTAATCCTTTGACCTCGTCGATTTCCGACCAATCTGGGCGTATAAT |
| | GAATCTAAACTTTAATTTCTTGTAATCATTCGAAATAATTTTTAGTTTGCATCCGTAGTTATCCCCTTTA |
| | TGTAACTGTAAATTTCTCAACGCGATATCTCCATTAATAATGATGTCGAATTCGTGCTGTATACCCATAC |
| | TGAATGGATGAACTAACGAATATCAACGGCGTTAATAGTAATTTACTTTTTCATCTTTACATATTGGGTA |
| | CTAGTTTTACTATCATAAGTTTATAAATTCCACAAGCTACTATGGAATAAGCCAACCATCTTAGTATACC |
| | ACACATGTCTTAAAGTTTATTAATTAATTACATGTTGTTTTATATATATCGCTACGAATTTAAAGAGAAA |
| | TCAGTTTAGGAAGAAAAAAATTATCTATCTACATCATCACGTCTCTGTATTCTACGATAGAGTGCTACTT |
| | TAAGATGAGACATATCCGTGTCATCAAAAATATACTCCATTAAAATGATTATTCCGGCAGCGAACTTGAT |
| | ATTGGATATATCACAACCTTTGTTAATATCTACGACAATAGACAGCAGTCCCATGGTTCCATAAACAGTG |
| | AGTTTATCTTTCTTTGAAGCGATAGTTTGTAGAGATCTTATAAAACCGTCAAACGACATCGCATTTATAT |
| | CTTTAGCTAATTCATATATGTTACCATCGTAATATCTAACCGCGTCTATCTTAAACGTTTCCATCGCTTT |
| | AAAGACGTTTCCGATAGATGGTCTCATTTCATCAGTCATACTGAGCCAACAAATATAATCGTGTATAACA |
| | TCTTTGATAGAATCAGACTCTAAAGAAAACGAATCGGCTTTATTATACGCATTCATGATAAACTTAATGA |
| | AAAATGTTTTTCGTTGTTTAAGTTGGATGAATAGTATGTCTTAATAATTGTTATTATTTCATTAATTAAT |
| | ATTTAGTAACGAGTACACTCTATAAAAACGAGAATGACATAACTAGTTATCAAAGTGTCTAGGACGCGTA |
| | ATTTTCATATGGTATAGATCCTGTAAGCATTGTCTGTATTCTGGAGCTATTTTCTTTATCGCATTAGTAA |
| | GTTCAGAATATGTTATAAATTTAAATCGAATAACGAACATGACTTTAGTAAAGTCGTCTATATTAACTCT |
| | TTTATTTTCTAGCCATCGTAATACCATGTTTAAGATAGTATATTCTCTAGTTACTACGATCTCATCGTTG |
| | TCTAGAATATCACATACTGAATCTACATCCAATTTTAGAAATTGGTCTGTGTTACATATCTCTTCTATAT |
| | TATTGTTGATGTATTGTCGTAGAAAACTATTACGTAGACCATTTTCTTTATAAAACGAATATATAGTACT |
| | CCAATTATCTTTACCGATATATTTGCACACATAATCCATTCTCTCAATCACTACATCTTTAAGATTTTCG |
| | TTGTTAAGATATTTGGCTAAACTATATAATTCTATTAGATCATCAACAGAATCAGTATATATTTTTCTAG |
| | ATCCAAAGACGAACTCTTTGGCGTCCTCTATAATATTCCCAGAAAAGATATTTTCGTGTTTTAGTTTATC |
| | GAGATCTGATCTGTTCATATACGCCATGATTGTACGGTACGTTATGATAACCGCATAAAATAAAAATCCA |
| | TTTTCATTTTTAACCAATACTATTCATAATTGAGATTGATGTAATACTTTGTTACTTTGAACGTAAAGAC |
| | AGTACACGGATCCGTATCTCCAACAAGCACGTAGTAATCAAATTTGGTGTTGTTAAACTTCGCAATATTC |
| | ATCAATTTAGATAGAAACTTATACTCATCATCTGTTTTAGGAATCCATGTATTATTACCACTTTCCAACT |
| | TATCATTATCCCAGGCTATGTTTCGTCCATCATCGTTGCGCAGAGTGAATAATTCTTTTGTATTCGGTAG |
| | TTCAAATATATGATCCATGCATAGATCGGCAAAGCTATTGTAGATGTGATTTTTCCTAAATCTAATATAA |
| | AACTCGTTTACTAGCAAACACTTTCCTGATTTATCGACCAAGACACATATGGTTTCTAAATCTATCAAGT |
| | GGTGGGGATCCATAGTTATGACGCAGTAACATAGATTATTACATTCTTGACTGTCGCTAATATCTAAATA |
| | TTTATTGTTATCGTATTGGATTCTGCATATAGATGGCTTGTATGTCAAAGATATAGAACACATAACCAAT |
| | TTATAGTCGCGCTTTACATTCTCGAATCTAAAGTTAAGAGATTTAGAAAACATTATATCCTCGGATGATG |
| | TTATCACTGTTTCTGGAGTAGGATATATTAAAGTCTTTACAGATTTCGTCCGATTCAAATAAATCACTAA |
| | ATAATATCCCACATTATCATCTGTTAGAGTAGTATCATTAAATCTATTATATTTTATGAAAGATATATCA |
| | CTGCTCACCTCTATATTTCGTACATTTTTAAACTGTTTGTATAATATCTCTCTGATACAATCAGATATAT |
| | CTATTGTGTCGGTAGACGATACCGTTACATTTGAATTAATGGTGTTCCATTTTACAACTTTTAACAAGTT |
| | GACCAATTCATTTCTAATAGTATCAAACTCTCCATGATTAAATATTTTAATAGTATCCATTTTATATCAC |
| | TACGGACACAAAGTAGCTGACATAAACCATTGTATAATTTTTATGTTTTATGTTTATTAGCGTACACATT |
| | TTGGAAGTTCCGGCTTCCATGTATTTCCTGGAGAGCAAGTAGATGATGAGGAACCAGATAGTTTATATCC |
| | GTACTTGCACTTAAAGTCTACATTGTCGTTGTATGAGTATGATCTTTTAAACCCGCTAGACAAGTATCCG |
| | TTTGATATTGTAGGATGTGGACATTTAACAATCTGACACGTGGGTGGATCGGACCATTCTCCTCCTGAAC |
| | ACAGGACACCAGAGTTACCAATCAACGAATATCCACTATTGCAACTATAAGTTACAACGCTCCCATCGGT |
| | ATAAAAATCCTCGTATCCGTTATGTCTTCCGTTGGATATAGATGGAGGGGATTGGCATTTAACAGATTCA |
| | CAAATAGGTGCCTCGGGATTCCATACCATAGATCCAGTAGATCCTAATTCACAATACGATTTAGATTCAC |
| | CGATCAAATGATATCCGCTATTACAAGAGTACGTTATACTAGAGCCAAAGTCTACTCCACCAATATCAAG |
| | TTGGCCATTATCGATATCTCGAGGCGATGGGCATCTCCGTTTAATACATTGATTAAAGAGTGTCCATCCA |
| | GTACCTGTACATTTAGCATATATAGGTCCCATTTTTTGCTTTCTGTATCCAGGTAGACATAGATATTCTA |
| | TAGTGTCTCCTATGTTGTAATTAGCATTAGCATCAGTCTCCACACTATTCTTAAATTTCATATTAATGGG |
| | TCGTGACGGAATAGTACAGCATGATAGAACGCATCCTATTCCCAACAATGTCAGGAACGTCACGCTCTCC |
| | ACCTTCATATTTATTTATCCGTAAAAATGTTATCCTGGACATCGTACAAATAATAAAAAGCCCATATATG |
| | TTCGCTATTGTAGAAATTGTTTTTCACAGTTGCTCAAAAACGATGGCAGTGACTTATGAGTTACGTTACA |
| | CTTTGGAGTCTCATCTTTAGTAAACATATCATAATATTCGATATTACGAGTTGACATATCGAACAAATTC |
| | CAAGTATTTGATTTTGGATAATATTCGTATTTTGCATCTGCTATAATTAAGATATAATCACCGCAAGAAC |
| | ACACGAACATCTTTCCTACATGGTTAAAGTACATGTATAATTCTATCCATTTGTCTTCCTTAACTATATA |
| | TTTGTATAGATAATTACGAGTCTCGTGAGTAATTCCAGTAATTACATAGATGTCGCCGTCGTACTCTACA |
| | GCATAAACTATACTATGATGTCTAGGCATGGGAGACTTTTTTATCCAACGATTTTTAGTGAAACATTCCA |
| | CATCGTTTAATACTACATATTTTTCATACGTGGTATAAACTCCACCCATTACATATATATCATCGTTTAC |
| | GAATACCGACGCGCCTGAATATCTAGGAGTAATTAAGTTTGGAAGTCTTATCCATTTCGAAGTGCCGTGT |
| | TTCAAATATTCTGCCACACCCGTTGAAATAGAAAATTCTAATCCTCCTATTACATATAACTTTCCATCGT |
| | TAACACAAGTACTAACTTCTGATTTTAACGACGACATATTAGTAACCGTTTTCCATTTTTTCGTTTCAAG |
| | ATCTACCCGCGATACGGAATAAACATGTCTATTGTTAATCATGCCGCCAATAATGTATAGACAATTATGT |
| | AAAACATTTGCATTATAGAATTGTCTATCTGTATTACCGACTATCGTCCAATATTCTGTTCTAGGAGAGT |
| | AATGGGTTATTGTGGATATATAATCAGAGTTTTTAATGACTACTATATTATGTTTTATACCATTTCGTGT |
| | CACTGGCTTTGTAGATTTGGATATAGTTAATCCCAACAATGATATAGCATTGCGCATAGTATTAGTCATA |
| | AACTTGGGATGTAAAATGTTGATGATATCTACATCGTTTGGATTTTTATGTATCCACTTTAATAATATCA |
| | TAGCTGTAACATCCTCATGATTTACGTTAACGTTTTCGTGGGATAAGATAGTTGTCAGTTCATCCTTTGA |
| | TAATTTTCCAAATTCTGGATCGGATGTCACCGCAGTAATATTGTTGATTATTTCTGACATCGACGCATTA |
| | TATAGTTTTTTAATTCCATATCTTTTAGAAAAGTTAAACATCCTTATACAATTTGTGGAATTAATATTAT |
| | GAATCATAGTTTTTACACATAGATCTACTACAGGCGGAACATCAATTATTATGGCAGCAACTAGTATCAT |
| | TTCTACATTGTTTATGGTGATGTTTATCTTCTTCCAGCGTATATAGTCTAATAGCGATTCAAACGCGTGA |
| | TAGTTTATACCATTCAATATAATCGCTTCATCCTTTAGATGGTGATCCTGAATTCGTTTAAAAAAATTAT |
| | ACGGAGATGCCGTAATAATTTCCTTATTCACTTGTATAATTTCCCCATTGATAGAAAATATCACGCTTTC |
| | CATTCTTGAAGTACTATAAGTAATTATAGTATAATGTAAAGGTTTATATATTCAATATTTTTTATAAAAA |
| | AATCATTTTGACATTAATTCCTTTTTAAATTTCCGTCTATCATCTATAGAAACGTATTCTATGAATTTAT |
| | AAAATGCTTTTACGTGTCCTATCGTAGGCGATAGAACCGCTAAAAAGCCTATCGAATTTCTACAAAAGAA |
| | TCTGTTATATGGTATAGGGAGAGTATAAAACATTAAATGTCCGTACTTATTAAAGTATTCAGTAGCCAAT |
| | CCTAACTCTTTCGAATACTTATTAATGGCTCTTGTTCTGTACGAATCTATTTTTTTGAACAACGGACCTA |
| | GTGGTATATCTTGTTCTATGTATCTAAAATAATGTCTGACTAGATCCGTTAGTTTAATATCCTCAGTCAT |
| | CTTGTCTAGAATGGCAAATCTAACTGCGGGTTTAGGCTTTAGTTTAGTTTTTATATCTACATCTATGTCT |
| | TTATCTAACACCAAAAATATAATAGCTAATATTTTATTACAATCATCCGGATATTCTTCTACGATCTCAC |
| | TAACTAATGTTTCTTTGGTTATACTAGTATAGTCACTATCGGACAAATAAAGAAAATCAGATGATCGATG |
| | AATAATACATTTAAATTCATCATCTGTAAGATTTTTGAGATGTCTCATTAAAATATTATTAGGGTCAGTA |
| | CTCATTATCATTCGGCAGCTATTACTTATTTTATTTTTCTGTATTTTATTATTTTTCACCATATAGATCA |
| | ATCATTAGATCATCAAAATATGTTTCAATCATCCTAAAGAGTATGGTGAATGACTCTTCCCATCTAATTT |
| | CTGAACGTTCACCAATGTCTCTAGCCACTTTGGCACTAATAGCGATCATTCGCTTAGCGTCTTCTATATT |
| | ATTAACTGGTTGATTCAATCTATCTAGCAATGGACCGTCGGACAGCGTCATTCTCATGTTCTTAATCAAT |
| | GTACATACATCGCCGTCATCTACCAATTCATCCAACAACATAAGCTTTTTAAAATCATCATTATAATAGG |
| | TTTGATCGTTGTCATTTCTCCAAAGAATATATCTAATAAGTAGAGTCCTCATGCTTAGTAATTTAACTAT |
| | TTTAGTTAACAACTATTTTTTATGTTAAATCAATTAGTACACCGCTATGTTTAATACTTATTCATATTTT |
| | AGTTTTTAGGATTGAGAATCAATACAAAAAATTAATGCATCATTAATTTTAGAAATACTTAGTTTCCACG |
| | TAGTTAATGAAACATTTGAACTCATCGTACAGGACGTTCTCGTACAGGACGTAACTATAAACCGGTTTAT |
| | ATTTGTTCAAGATAGATACAAATCCGATAACTTTTTTTACGAATTCTACGGGATCCACTTTAAAAGTGTC |
| | ATACCGGGTTCTTTTTATTCTTTTAAACAGATCAATGGTGTGATGTTGATTAGGTCTTTTACGAATTTGA |
| | TATAGAATAGCGTTCACATATCCTCCATAATGGTCAATCGCCATTTGTTCGTATGTCATAAATTCTTTAA |
| | TTATATGACACTGTGTATTATTTAGTTCATCCTTGTTCATCATTAGGAATCTATCCAAAATGGCAATTAT |
| | ACTAGAACTATAGGTGCGTTGTATACACATATTGATGTGTCTGTTTATACAATCCATGATATTTGGATCC |
| | ATGCTACTACCTTCGGGTAAAATTGTAGCATCATATACCATTTCTAGTACTTTAGGTTCATTATTATCCA |
| | TTGCAGAGGACGTCATGATCGAATCCTAAAAAAATATATTATTTTTATGTTATTTTGTTAAAAATAATCA |
| | TCGAATACTTCGTAAGATACTCCTTCATGAACATAATCAGTTACAAAACGTTTATATGAAGTAAAGTATC |
| | TACGATTTTTACAAAAGTCCGGATGCATAAGTACAAAGTACGCGATAAACGGAATAATAATAGATTTATC |
| | TAGTCTATCTTTTTCTATAGCTTTCATAGTTAGATACATGGTCTCAGAAGTAGGATTATGTAACATCAGC |
| | TTCGATAAAATGACTGGGTTATTTAGTCTTACACATTCGCTCATACATGTATGACCGTTAACTACAAAGT |
| | CTACACTAAAATGATTGAACAATAGATAGTCTACCATTGTTTCGTATTCAGATAGTACAGCGTAGTACAT |
| | AGCATCTTCACAAATTATATCATTGTCTAATAGATATTTGACGCATCTTATGGATCCCACTTCAACAGCC |
| | ATCTTAAAATCGGTAGAATCATATTGCTTTCCTTTATCATTAATAATTTCTAAAACATCATCTCTATCAT |
| | AAAAGATACAAATATTAACTGTTTGATCCGTAATAACATTGCTAGTCGATAGCAATTTGTTAATAAGATG |
| | CGCTGGGCTCAATGTCTTAATAAGAAGTGTAAGAGGACTATCTCCGAATTTGTTTTGTTTATTAACATCC |
| | GTTGATGGAAGTAAAAGATCTATAATGTCTACATTCTTGACTGTTTTAGAGCATACAATATGGAGAGGTG |
| | TATTTCCATCATGATCTGGTTTTGAGGGACTAATTCCTAGTTTCATCATCCATGAGATTGTAGAAGCTTT |
| | TGGATTGTCTGACATAAGATGTCTATGAATATGATTTTTGCCAAATTTATCCACTATCCTGGCTTCGAAT |
| | CCGATGGACATTATTTTTTTAAACACTCTTTCTGAAGGATCTGTACACGCCAACAACGGACCACATCCTT |
| | CTTCATCAACCGAGTTGTTAATCTTGGCTCCATACTGTACCAATAAATTTATTCTCTCTATGACTTCATC |
| | ATCTGTTCCCGAGAGATAATATAGAGGTGTTTTATTATGTTTATCACACGCGTTTGGATCTGCGCCGTGC |
| | GTCAGCAGCATCGCGACTATTCTATTATTATTAATTTTAGAAGCTATATGCAATGGATAATTTCCATCAT |
| | CATCCGTCTCATTTGGAGAGTATCCTCTATGAAGAAGTTCTTCGACAAATCGTTCATCTAGTCCTTTAAT |
| | TCCACAATACGCATGTAGAATGTGATAATTATTTCCAGAAGGTTCGATAGCTTGTAGCATATTCCTAAAT |
| | ACATCTAAATTTTTACTATTATATTTGGCATAAAGAGATAGATAATACTCGGCCGACATAATGTTGTCCA |
| | TTGTAGTATAAAAATTAATATTTCTATTTCTATTTCTGTATATTTGCAACAATTTACTCTCTATAACAAA |
| | TATCATAACTTAGTTCTTTTATGTCAAGAAGGCACTGGTTTAGTTCATCTATAAATGTCACGCCATAACT |
| | ACCACGCATGCTATACTCAGAATTATGATAAAGATATTTATCCTTGGGGTGTAGGTAATGGGGATTAATC |
| | TTTGTTGGATCAGTCTCTAAGTTAACACATGTCACACATGATCCATTTATAGTTATATCACACGATGATG |
| | ATTTATGAATTGATTCCGGAAGATCGCTATCGTATTTTGTGGTTCCACAATTCATTTCCATACATGTTAT |
| | TGTCACACTAATATTATGATGAACTTTATCTAGCCGCTGAGTGGTAAACAACAGAACAGATAGTTTATTA |
| | TCTTTACCAACACCCTCAGCCGCTGCCACAAATCTCTGATCCGTATCCATGATGGTCATGTTTATTTCTA |
| | GTCCGTATCCAGTCAACACTATGTTAGCATTTCTGTCGATATAGCTTTCACTCATATGACACTCACCAAT |
| | AATAGTAGAATTAATGTCGTAATTTACACCAATAGTGAGTTCGGCGGCAAAGTACCAATACCGGTAATCT |
| | TGTCGAGGAGGACATATAGTATTCTTGTATTCTACCGAATACCCGAGAGATGCGATACAAAAGAGTAAGA |
| | CTAATTTGTAAACCATCTTACTCAAAATATGTAACAATAGTACGATGCAATGAGTAAGACAATAGGAAAT |
| | CTATCTTATATACACATAATTATTCTATCAATTTTACCAATTAGTTAGTGTAATGTTAACAAAAATGTGG |
| | GAGAATCTAATTAGTTTTTCTTTACACAATTGACGTACATGAGTCTGAGTTCCTTGTTTTTGCTAATTAT |
| | TTCATCCAATTTATTATTCTTGACTATATCGAGATCTTTTGTATAGGAGTCAGACTTGTATTCAACATGC |
| | TTTTCTATAATCATTTTAGCTATTTCGGCATCATCCAATAGTACATTTTCCAGATTAGCAGAATAGATAT |
| | TAATGTCGTATTTGAACAGAGCCTGTAACATCTCAATGTCTTTATTATCTATAGCCAATTTAATGTCCGG |
| | AATGAAGAGAAGGGAATTATTGGTGTTTGTCGACGTCATATAGTCGAGCAAGAGAATCATCATATCCACG |
| | TGTCCATTTTTTATAGTGATGTGAATACAACTAAGGAGAATAGCCAGATCAAAAGTAGATGGTATCTCTG |
| | AAAGAAAGTAGGAAACAATACTTACATCATTAAGCATGACGGCATGATAAAATGAAGTTTTCCATCCAGT |
| | TTTCCCATAGAACATCAGTCTCCAATTTTTCTTAACAAACAGTTTTACCGTTTGCATGTTACCACTATCA |
| | ACCGCATAATACAATGCGGTGTTTCCCTTGTCATCAAATTGTGAATCATCCAGTCCACTGAATAGCAAAA |
| | TCTTTACTATTTTGGTATCTTCCAATGTGGCTGCCTGATGTAATGGAAATTCATTCTCTAGAAGATTTTT |
| | CAATGCTCCAGCGTTCAACAACGTACATACTAGACGCACGTTATTATCAGCTATTGCATAATACAAGGCA |
| | CTATGTCCATGGACATCCGCCTTAAATGCATCTTTGCTAGAGAGAAAGCTTTTCAGCTGCTTAGACTTCC |
| | AAGTATTAATTCGTGACAGATCCATGTCTGAAACGAGACGCTAATTAGTGTATATTTTTTCATTTTTTAT |
| | AATTTTGTCATATTGCACCAGAATTAATAATATCTCTAATAGATCTGATTAGTAGATACATGGCTATCGC |
| | AAAACAACATATACACATTTAATAAAAATAATATTTATTAAGAAAATTCAGATTTCACGTACCCATCAAT |
| | ATAAATAAAATAATGATTCCTTACACCGTACCCATATTAAGGAGATTCCACCTTACCCATAAACAATATA |
| | AATCCAGTAATATCATGTCTGATGATGAACACAAATGGTGTATTAAATTCCAGTTTTTCAGGAGATGATC |
| | TCGCCGTAGCTACCATAATAGTAGATGCCTCTGCTACAGTTCCTTGTTCGTCGACATCTATCTTTGCATT |
| | CTGAAACATTTTATAAATATATAATGGGTCCCTAGTCATATGTTTAAACGACGCATTATCTGGATTAAAC |
| | ATACTAGGAGCCATCATTTCGGCTATCGACTTAATATCCCTCTTATTTTCGATAGAAAATTTAGGGAGTT |
| | TAAGATTGTACACTTTATTCCCTAATTGAAACGACCAATAGTCTAATTTTGCAGCCGTAATAGAATCTGT |
| | GAAATGGGTCATATTATCACCTATTGCCAGGTACATACTAATATTAGCATCCTTATACGGAAGGCGTACC |
| | ATATCATATTCTTCGTCATCGATTGTGATTGTATTTCCTTGCAATTTAGTAACTACGTTCATCATGGGAA |
| | CCGTTTTCGTACCGTACTTATTAGTAAAACTAGCATTGCGTGTTTTAGTGATATCAAACGGATATTGCCA |
| | TATACCTTTAAAATATATAGTATTAATGATTGCCCATAGAGTATTATTGTCGAGCATATTAGAATCTACT |
| | ACATTAGACATACCGGATCTACGTTCTACTATAGAATTAATTTTATTAACCGCATCTCGTCTAAAGTTTA |
| | ATCTATATAGGCCGAATCTATGATATTGTTGATAATACGACGGTTTAATGCACACAGTATTATCTACGAA |
| | ACTTTGATAAGTTAGATCAGTGTACGTATATTTAGATGTTTTCAGCTTAGCTAATCCTGATATTAATTCT |
| | GTAAATGCTGGACCCAGATCTCTTTTTCTCAAATCCATAGTCTTCAATAATTCTATTCTAGTATTACCTG |
| | ATGCAGGCAATAGCGACATAAACATAGAAAACGAATAACCAAACGGTGAGAAGACAATATTATCATCTTG |
| | AATATTTTTATACGCTACTATACCGGCATTGGTAAATCCTTGCAGACGATAGGTAGACACTGAACACGTT |
| | AACGATAGTATCAATAACGCAATCATGATTTTATGGTATTAATAATTAACCTTATTTTTATGTTCGGTAT |
| | AAAAATTATTGATGTCTACACATCCTTTTGTAATTGACATCTATATATCCTTTTGTATAATCAACTCTAA |
| | TCACTTTAACTTTTACAGTTTTCCCTACCAGTTTATCCCTATATTCAACATATCTATCCATATGCATCTT |
| | AACACTCTCTGCCAAGATAGCTTCAAAGTGAGGATAGTCAAAAAGATAAATATATAGAGCATAATCCTTC |
| | TCGTATACTCTGCCCTTTATTACATCACCCGCATTGGGCAACGAATAACAAAATGCAAGCATCTTGTTAA |
| | CGGGCTCGTAAATTGGGATAAAAATTATGTTTTTATATCTATTTTATTCAAGAGAATATTCAGGAATTTC |
| | TTTTTCCGGTTGTATCTCATCGCAGTATATATCATTTGTACATTGTTTCATATTTTTTAATAGTCTACAC |
| | CTTTTAGTAGGACTAGTATCGTACAATTCATAGCTGTATTTTGAATTCCAATCACGCATAAAAATATCTT |
| | CCAATTGTTGACGAAGACCTAATCCATCATCCGGTGTAATATTAATAGATGCTCCACATGTATCCGTAAA |
| | GTAATTTCCTGTCCAATTTGAGGTACCTATATACGCCGTTTTATCGGTTACCATATATTTGGCATGGTTT |
| | ACCCTAGAATACGGAATGGGAGGATCAGCATCTGGTACAATAAATAGCTTTACTTCTATATTTATGTTTT |
| | TAGATTTTAGCATAGCGATAGATCTTAAAAAGTTTCTCATGATAAACGAAGATCGTTGCCAGCAACTAAT |
| | CAATAGCTTAACTGACACTTGTCTGTCTATAGCGGCTCTTCTTAATTCATCTTCTATATAAGGCCAAAAC |
| | AAAATATTGCCTGCCTTCGAATAAATAATAGGGATAAAGTTCATAACAGATACATAAACGAATTTACTCG |
| | CATTTCTGATACATGACAATAAAGCGGTTAAATCATTGGTTCTTTCCATAGTACATAGTTGTTGCGGTGC |
| | AGAAGCAATAAATACAGAGTGTGGAACGCCGCTTACGTTAATACTAAGAGGATGATCTGTATTATAATAC |
| | GACGGATAAAAGTTTTTCCAATTATATGGTAGATTGTTAACTCCAAGATACCAGTATACCTCAAAAATTT |
| | GAGTGAGATCCGCTGCCAAGTTCCTATTATTGAAGATCGCAATACCCAATTCTTTGACCTGAGTTAGTGA |
| | TCTCCAATCCATGTTAGCGCTTCCTAAATAAATATGTGTATTATCAGATATCCAAAATTTTGTATGAAGA |
| | ACTCCTCCTAGGATATTTGTAATATCTATGTATCGTACTTCAACTCCGGCCATTTGTAGTCTTTCAACAT |
| | CCTTTAATGGTTTGTTAGATTTATTGACGGCTACTCTAACTCGTACTCCTCTTTTGGGTAATTGTACAAT |
| | CTTGTTTAATATTATCGTGCCGAAATTCGTACCCACTTCATCCGATAAACTCCAATAAAAAGATGATATA |
| | TCTAGTGTTTTTGTGGTATTGGATAGAATTTCCCTCCACATGTTAAATGTAGACAAATATACTTTATCAA |
| | ATTGCATACCTATAGGAATAGTCTCTGTAATCACTGCGATTGTATTATCCGGATTCATTTTATTTGTTAA |
| | AAGAATAATCCTATATCACTTCACTCTATTAAAAATCCAAGTTTCTATTTCTTTCATGACTGATTTTTTA |
| | ACTTCATCCGTTTCCTTATGAAGATGATGTTTGGCACCTTCATAAATTTTTATTTCTCTATTACAATTTG |
| | CATGTTGCATGAAATAATATGCACCTAAAACATCGCTAATCTTATTGTTTGTTCCCTGGAGTATGAGAGT |
| | CGGGGGGTGTTAATCTTGGAAATTATTTTTCTAACCTTGTTGGTAGCCTTCAAGACCTGACTAGCAAATC |
| | CAGCCTTAATTTTTTCATGATTGATTAATGGGTCGTATTGGTATTTATAAACTTTATCCATATCTCTAGA |
| | TACTGATTCTGGACATAGCTTTCCGACTGGCGCATTTAGTGTGATGGTTCCCATAAGTTTGGCAGCTAGC |
| | AGATTCAGTTTTGAAACAGCATCTGCATTAACTAGAGGAGACATTAGAATCATTGCTGTAAACAAGTTTG |
| | GATTATCGTAAGAGGCTAGCTCCCATGGAATGACCCAATAAGTAGATTTAATAGTTACCACGTGCTGTAC |
| | CAAAGTCATCAATCATCATTTTTTCACCATTACTTCTTCCATGTCCAATATGATCATGTGAGAATACTAA |
| | AATTCCTAACGATGATATGTTTTCAGCTAGTTCGTCATAACGTCCAGAATGTTTACCAGCTCCATGACTT |
| | ATGAATACTAATGCCTTAGGATATGTAATAGGTTTCCAATATATGTAATCATTGTCCAGATTGAACATAC |
| | AGTTTGCACTCATGATTCACGTTATATAACTATCAATATTAACAGTTCGTTTGATGATCATATTATTTTT |
| | ATGTTTTATTGATAATTGTAAAAACATACAATTAAATCAATATAGAGGAAGGAGACGGCTACTGTCTTTT |
| | GTGAGATAGTCATGGCGACTAAATTAGATTATGAGGATGCTGTTTTTTACTTTGTGGATGATGATAAAAT |
| | ATGTAGTCGCGACTCCATCATCGATCTAATAGATGAATATATTACGTGGAGAAATCATGTTATAGTGTTT |
| | AACAAAGATATTACCAGTTGTGGAAGACTGTACAAGGAATTGATGAAGTTCGATGATGTCGCTATACGGT |
| | ACTATGGTATTGATAAAATTAATGAGATTGTCGAAGCTATGAGCGAAGGAGACCACTACATCAATTTTAC |
| | AAAAGTCCATGATCAGGAAAGTTTATTCGCTACCATAGGAATATGTGCTAAAATCACTGAACATTGGGGA |
| | TACAAAAAGATTTCAGAATCTAGATTCCAATCATTGGGAAACATTACAGATCTGATGACCGACGATAATA |
| | TAAACATCTTGATACTTTTTCTAGAAAAAAAATTGAATTGATGATATAGGGGTCTTCATAACGCATAATT |
| | ATTACGTTAGCATTCTATATCCGTGTTAAAAAAAATTATCCTATCATGTATTTGAGAGTTTTATATGTAG |
| | CAAACATGATAGCTGTGATGCCAATAAGCTTTAGATATTCACGCGTGCTAGTGTTAGGGATGGTATTATC |
| | TGGTGGTGAAATGTCCGTTATATAATCTACAAAATAATCATCGCATATAGTATGCGATAGTAGAGTAAAC |
| | ATTTTTATCGTTTCTACTGGGTTCATACATCGTCTACCCAATTCGGTTATAAATGAAATTGTCGCCAATC |
| | TTACACCCAACCCCTTGTTATCCATTAGTATAGTATTAACTTCGTTATTTATGTCATAAACTGTAAATGA |
| | TTTTGTAGATGCCATATCATACATGATATTCATGTCCCTATTATAATCATTACTAACTTTATCACAATAT |
| | ATGTTGATAATATCTATATATGATCTAGTCTTTGTGGGCAACTGTCTATACAAGTCGTCTAAACGTTGTT |
| | TACTCATATAGTATCGAACAGCCATCATTACATGGTCCCGTTCCGTTGATAGATAATCGAGTATGTTAGT |
| | GGACTTGTCAAATCTATATACCATATTTTCTGGAAGTGGATATACATAGTCGTGATCAACATTATTGCTA |
| | GCCTCATCTTCTATATCCTGTACTATACCATTATCTATATCATCTACATAATCTACGATATTATTACACA |
| | TAAACATCGACAACATACTATTGTTTATTATCTAAGTCCTGTTGATCCAAACCCTTGATCTCCTCTATTT |
| | GTACTATCTAGAGATTGTACTTCTTCCAGTTCTGGATAATATATACGTTGATAGATTAGCTGAGCTATTC |
| | TATCTCCAGTATTTACATTAAACGTACATTTTCCATTATTAATAAGAATGACTCCTATGTTTCCCCTATA |
| | ATCTTCGTCTATTACACCGCCTCCTATATCAATGCCTTTTAGGGACAGACCAGACCTAGGAGCTATTCTA |
| | CCATAGCAGAACTTAGGCATGGACATACTAATATCTGTCTTAATTAACTGTCGTTCTCCTGGAGGGATAG |
| | TATAATCGTAAGCGCTATATAAATCATATCCGGCGGCGTAAGGTGATTGCCTAGTAGGAGATTTAGCTCT |
| | GTTAGTTTCCTTAACAAATCTAACTGGTGAGTTAATATTCATGTTGAACATAAAACTAATATTTTATTTC |
| | AAAATTATTTACCATCCCATATATTCCATGAATAAGTGTGATGATTGTACACTTCTATAGTATCTATATA |
| | CGATTCACGATAAAATCCTCCTATCAATAGCAGTTTATTATCCACTATGATCAATTCTGGATTATCCCTC |
| | GGATAAATAGGATCATCTATCAGAGTCCATGTATTGCTGGATTCACAATAAAATTCCGCATTTCTACCAA |
| | CCAAGAATAACCTTCTACCGAACACTAACGCGCATGATTTATAATGAGGATAATAAGTGGATGGTCCAAA |
| | CTGCCACTGATCATGATTGGGTAGCAAATATTCTGTAGTTGTATCAGTTTCAGAATGTCCTCCCATTACG |
| | TATATAACATTGTTTATGGATGCCACTGCTGGATTACATCTAGGTTTCAGAAGACTCGGCATATTAACCC |
| | AAGCAGCATCCCCGTGGAACCAACGCTCAACAGATGTGGGATTTGGTAGACCTCCTACTACGTATAATTT |
| | ATTGTTAGCGGGTATCCCGCTAGCATACAGTCTGGGGCTATTCATCGGAGGAATTGGAATCCAATTGTTT |
| | GATATATAATTTACCGCTATAGCATTGTTATGTATTTCATTGTTCATCCATCCACCGATGAGATATACTA |
| | CTTCTCCAACATGAGTACTTGTACACATATGGAATATATCTATAATTTGATCCATGTTCATAGGATACTC |
| | TATGAATGGATACTTGTATGATTTGCGTGGTTGTTTATCACAATGAAATATTTTGGTACAGTCTAGTATC |
| | CATTTTACATTATTTATACCTCTGGGAGAAAGATAATTTGACCTGATTACATTTTTGATAAGGAGTAGCA |
| | GATTTCCTAATTTATTTCTTCGCTTTATATACCACTTAATGACAAAATCAACTACATAATCCTCATCTGG |
| | AACATTTAGTTCATCGCTTTCTAGAATAAGTTTCATAGATAGATAATCAAAATTGTCTATGATGTCATCT |
| | TCCAGTTCCAAAAAGTGTTTGGCAATAAAGTTTTTAGTATGACATAAGAGATTGGATAGTCCGTATTCTA |
| | TACCCATCATGTAACACTCGACACAATATTCCTTTCTAAAATCTCGTAAGATAAAGTTTATACAAGTGTA |
| | GATGATAAATTCTACAGAGGTTAATATAGAAGCACGTAATAAATTGACGACGTTATGACTATCTATATAT |
| | ACCTTTCCAGTATACGAGTAAATAACTATAGAAGTTAAACTGTGAATGTCAAGGTCTAGACAAACCCTTG |
| | TAACTGGATCTTTATTTTTCGTGTATTTTTGACGTAAATGTGTGCGAAAGTAAGGAGATAACTTTTTCAA |
| | TATCGTAGAATTGACTATTATATTGCCACCTATAGCATCAATAATTGTTTTGAATTTCTTAGTCATAGAC |
| | AATGCTAATATATTCTTACAGTACACAGTATTAACAAATATCGGCATTTATGTTTCTTTAAAAGTCAACA |
| | TCTAAAGAAAAATGATTATCTTCTTGAGACATAACTCCCATTTTTTGGTATTCACCCACACGTTTTTCGA |
| | AAAAATTAGTTTTTCCTTCCAATGATATATTTTCCATGAAATCAAACGGATTGGTAACATTATAAATTTT |
| | TTTAAATCCCAATTCAGAAATCAATCTATCCGCGACGAATTCTATATATGTTTTCATCATTTCACAATTC |
| | ATTCCTATAAGTTTAACTGGAAGAGCCGCAGTAAGAAATTCTTGTTCAATGGATACCGCATCTGTTATAA |
| | TAGATCTAACGGTTTCTTCACTCGGTGGATGCAATAAATGTTTAAACATCAAACATGCGAAATCGCAGTG |
| | CAGACCCTCGTCTCTACTAATTAGTTCGTTGGAAAACGTGAGTCCGGGCATTAGGCCACGCTTTTTAAGC |
| | CAAAATATGGAAGCGAATGATCCAGAAAAGAAGATTCCTTCTACTGCAGCAAAGGCAATAAGTCTCTCTC |
| | CATAACCGGCGCTGTCATGTATCCACTTTTGAGCCCAATCGGCCTTCTTTTTTACACAAGGCATCGTTTC |
| | TATGGCATTAAAGAGATAGTTTTTTTCATTACTATCTTTAACATAAGTATCGATCAAAAGACTATACATT |
| | TCCGAATGAATGTTTTCAATGGCCATCTGAAATCCGTAGAAACATCTAGCCTCGGTAATCTGTACTTCTG |
| | TACAAAATCGTTCCGCCAAATTTTCATTCACTATTCCGTCACTGGCTGCAAAAAACGCCAATACATGTTT |
| | TATAAAATATTTTTCGTCTGGTGTTAGTTTATTCCAATCATTGATATCTTTAGATATATCTACTTCTTCC |
| | ACTGTCCAAAATGATGCCTCTGCCTTTTTATACATGTTCCAGATGTCATAATATTGGATTGGGAAAATAA |
| | CAAATCTATTTGGATTTGGTGCAAGGATGGGTTCCATAACTAAATTAACAATAACAATAAATTTTTTTTC |
| | AGTTATCTATATGCCTGTACTTGGATCTTTTGTACATCGATATCGCCGCAATCACTACAATAATTACAAG |
| | TATTATTGATAGCATTGTTATTAGTACTATCATAATTAAATTATCGACATTCATGGGTGCTGAATAATCG |
| | TTATTATCATCATTATCATTTTGTAATTGTGACATCATACTAGATAAATCGTTTGCGAGATTGTTGTGGG |
| | AAGCGGGCATGGAGGATGAATTATCGTTATTATTATTTAACGCCTCCCATTCGGATTCACAAATGTTACG |
| | CACATTCAACATTTTATGGAAACTATAATTTTGTGAAAACAGATAACAAGAAAACTCGTCATCGTTCAAA |
| | TTTTTAACGATAGTAAACCGATTAAACGTCGAGCTAATTTCTAACGCTAGCGACTCTGTTGGATATGGGT |
| | TTCCAGATATATATCTTTTCAGTTCCCCTACGTATCTATAATCATCTGTAGGAAATGGAAGATATTTCCA |
| | TTTATCTACTGTTCCTAATATCATATGTGGTGGTGTAGTAGAACCATTAAGCGCGAAAGATGTTATTTCG |
| | CATCGTATTTTAACTTCGCAATAATTTCTGGTTAGATAACGCACTCTACCAGTCAAGTCAATGATATTAG |
| | CCTTTACAGATATATTCATAGTAGTTGTAACGATGACTCCATCTTTTAGATGCGATACTCCTTTGTATGT |
| | ACCAGAATCTTCGTACCGCAAACTCGATATATTTAAACAAGTTAATGAGATATTAACGCGTTTTATGAAT |
| | GATGATATATAACCAGAAGTTTTATCCTCGGTGGCTAGCGCTATAACCTTATCATTATAATACCAACTAG |
| | TGTGATTAATATGTGACACGTTAGTGTGGGTACAAATATGTACATTATCGTCTACGTCGTATTCGATACA |
| | TCCGCATACAGCCAACAAATATAAAATGACAAATACTCTAACGCCGTTCGTACCCATCTTGATGCGGTTT |
| | AATAAATGTTTTGATTTCAATTTATTGTAAAAAAAGATTCGGTTTTATACTGTTCGATATTCTCATTGCT |
| | TATATTTTCATCTATCATCTCCACACAGTCAAATCCGTGGTTAGCATGCACCTCATCAACCGGTAAAAGA |
| | CTATCGGACTCTTCTATCATTATAACTCTAGAATATTTAATTTGGTCATTATTAATCAAGTCAATTATCT |
| | TATTTTTAACAAACGTGAGTATTTTACTCATTTTTTATAAAAACTTTTAGAAATATACAGACTCTATCGT |
| | GTGTCTATATCTTCTTTTTATATCCAATGTATTTATGTCTGATTTTTCTTCATTTATCATATATAATGGT |
| | CCAAATTCTACACGTGCTTCGGATTCATCCAGATCATTAAGGTTCTTATAATTGTAACATCCTTCTCTTC |
| | CCTCTTCTACATCTTCCTTCTTATTCTTATTCTTAGCGTCACAGAATCTACCACAGCAGGATCCCATGAC |
| | GAGCGTCATATTAAACTAATCCATTTTCAATTATAATATACGATTAGTAATGACCATTAAAATAAAAAAT |
| | ATTCTTCATAACCGGCAAGAAAGTGAAAAGTTCACATTGAAACTATGTCAGTAGTATACATCATGAAATG |
| | ATGATATATATATACTCTATTTTGGTGGAGGATTATATGATATAATTCGTGGATAATCATTCTTAAGACA |
| | CATTTCTTCATTCGTAAATCTTTTCACGTTAAATGAGTGTCCATATTTTGCAATTTCTTCATATGATGGC |
| | GGTGTACGTGGACGAAGCTGCTCCTGTTCTTGTTGTAGTCGCCGACTGTCGTGTTTGCGTTTAGATCCCT |
| | CCATTATCGCGATTGCGTAGATGGAGTACTATTATATACCTTGTAATTAAATTTTTTTATTAATTAAACG |
| | TATAAAAACGTTCCGTATCTGTATTTAAGAGCCAGATTTCGTCTAATAGAACAAATAGCTACAGTAAAAA |
| | TAACTAGAATAATTGCTACACCCACTAGAAACCACGGATCGTAATACGGCAATCGGTTTTCGATAATAGG |
| | TGGAACGTATATTTTATTTAAGGACTTAACAATTGTCTGTAAACCACAATTTGCTTCCGCGGATCCTGTA |
| | TTAACTATCTGTAAAAGCATATGTTGACCGGGCGGAGCCGAACATTCTCCGATATCTAATTTCTGTATAT |
| | CTATAATATTATTAACCTCCGCATACGCATTACAGTTCTTTTCTAGCTTGGATACCGCACTAGGTACATC |
| | GTCTAGATCTATTCCTATTTCTTCAGCGATAGCTCTTCTATCCTTTTCCGGAAGCAATGAAATCACTTCA |
| | ATAAATGATTCAACCATGAGTGTGAAACTAAGTCGAGAATTACTCATGCATTTGTTAGTTATTCGGAGCG |
| | CGCAATTTTTAAACTGTCCTATAACCTCTCCTATATGAATAGCACAAGTGACATTAGTAGGGATAGAATG |
| | TTGAGCTAATTTTTGTAAATAACTATCTATAAAAAGATTATACAAAGTTTTAAACTCTTTAGTTTCCGCC |
| | ATTTATCCAGTCTGAGAAAATGTCTCTCATAATAAATTTTTCCAAGAAACTAATTGGGTGAAGAATGGAA |
| | ACCTTTAATCTATATTTATCACAGTCTGTTTTGGTACACATGATGAATTCTTCTAATGCTGTACTAAATT |
| | CGATATCTTTTTCGATTTCTGGATATGTTTTTAATAAAGTATGAACAAAGAAATGGAAATCGTAATACCA |
| | GTTATGTTCAACTTTGAAATTGTTTTTTATTTTCTTGTTAATGATTCCAGCCACTTGGGAAAAGTCAAAG |
| | TCGTTTAATGCCGATTTAATACGTTCATTAAAAACAAACTTTTTATCCTTTAGATGAATTATTATTGGTT |
| | CATTGGAATCAAAAAGTAAGATATTATCGGGTTTAAGATCTGCGTGTAAAAAGTTGTCGCAACAGGGTAG |
| | TTCGTAGATTTTAATGTATAACAGAGCCATCTGTAAAAAGATAAACTTTATGTATTGTACCAAAGATTTA |
| | AATCCTAATTTGATAGCTAACTCGGTATCTACTTTATCTGCCGAATACAGTGCTAGGGGAAAAATTATAA |
| | TATTTCCTCTTTCGTATTCGTAGTTAGTTCTCTTTTCATGTTCGAAAAAGTGAAACATGCGGTTAAAATA |
| | GTTTATAACATTAATATTACTGTTAATAACTGCCGGATAAAAGTGGGATAGTAATTTCACGAATTTGATA |
| | CTGTCCTTTCTCTCGTTAAACGCCTTTAAAAAAACTTTAGAAGAATATCTCAATGAGAGTTCCTGACCAT |
| | CCATAGTTTGTATCAATAATAGCAACATATGAAGAACCCGTTTATACAGAGTATGTAAAAATGTTAATTT |
| | ATAGTTTAATCCCATGGCCCACGCACACACGATTAATTTTTTTTCATCTCCCTTTAGATTGTTGTATAGA |
| | AATTTGGGTACTGTGAACTCCGCCGTAGTTTCCATGGGACTATATAATTTTGTGGCCTCGAATACAAATT |
| | TTACTACATAGTTATCTATCTTAAAGACTATACCATATCCTCCTGTAGATATGTGATAAAAATCGTCGTT |
| | TATAGGATAAAATCGTTTATCCTTTTGTTGGAAAAAGGATGAATTAATGTAATCATTCTCTTCTATCTTT |
| | AGTAGTGTTTCCTTATTAAAATTCTTAAAATAATTTAACAATCTAACTGACGGAGCCCAATTTTGGTGTA |
| | AATCTAATTGGGACATTATGTTGTTAAAATACAAACAGTCTCCTAATATAACAGTATCTGATAATCTATG |
| | GGGAGACATCCATTGATATTCAGGGGATGAATCATTGGCAACACCCATTTATTGTACAAAAAGCCCCAAT |
| | TTACAAACGAAAGTCCAGGTTTGATAGAGACAAACAATTAACTATTTTGTCTCTGTTTTTAATTTCTTTG |
| | GTAATGAAATTATTCACAATATCAGTATCTTCTTTATCTACCAGAGATTTTACTAACTTGATAACCTTGG |
| | CTGTCTCATTCAATAGGGTAGTAATATTTGTATGTGTGATATTGATATCTTTTTGAATTGTTTCTTTTAG |
| | AAGTGATTCTTTGATGGTGCCAGCATACGAATTACAATAATGCAGAAACTCAGTTAACATGCAGGAATTA |
| | TAGTAAGCCAATTCCAATTGTTGCCTGTATTGTATTAGAGTATTAATATGCGCAATGGTGTCCTTGCGTT |
| | TCTCTGATAGAATGCGAGCAGCGATTTTGGCGTTATCATTTGACGATATTTCTGGAATGACGAATCCTGT |
| | TTCTACTAACTTTTTGGTAGGACAAAGTGAAACAATCAAGAAGATAGCTTCTCCTCCTATTTGTGGAAGA |
| | AATTGAACTCCTCTAGATGATCTACTGACGATAGTATCTCCTTGACAGATATTGGACCGAATTACAGAAG |
| | TACCTGGAATGTAAAGCCCTGAAACCCCCTCATTTTTTAAGCAGATTGTTGCCGTAAATCCTGCACTGTG |
| | ACCAAGATAGAGAGCTCCTTTGGTGAATCCATCTCTATGTTTCAGTTTAACCAAGAAACAGTCAGCTGGT |
| | CTAAAATTTCCATCTCTATCTAATACAGCATCTAACTTGATGTCAGGAACTATGACCGGTTTAATGTTAT |
| | ATGTAACATTGAGTAAATCCTTAAGTTCATAATCATCACTGTCATCAGTTATGTACGATCCAAACAATGT |
| | TTCTACTGGCATAGTGGATACGAAGATGCTATCCATCAGAATGTTTCCCTGATTAGTATTTTCTATATAG |
| | CTATTCTTCTTTAAACGATTTTCCAAATCAGTAACTATGTTCATTTTTTTAGGAGTAGGACGCCTAGCCA |
| | GTATGGAAGAGGATTTTCTAGATCCTCTCTTCAACATCTTTGATCTCGATGGAATGCAAAACCCCATAGT |
| | GAAACAACCAACGATAAAAATAATATTGTTTTTCACTTTTTATAATTTTACCATCTGACTCATGGATTCA |
| | TTAATATCTTTATAAGAGCTACTAACGTATAATTCTTTATAACTGAACTGAGATATATACACCGGATCTA |
| | TGGTTTCCATAATTGAGTAAATGAATGCTCGGCAATAACTAATGGCAAATGTATAGAACAACGAAATTAT |
| | ACTAGAGTTGTTAAAGTTAATATTTTCTATGAGCTGTTCCAATAAATTATTTGTTGTAACTGCGTTCAAG |
| | TCATAAATCATCTTGATACTATCCAGTAAACCGTGTTTAAGTTCTGGAATATTATCATCCCATTGTAAAG |
| | CCCCTAATTCGACTATCGAATATCCTGCTCTGATAGCAGTTTCAATATCGACGGACGTCAATACTGTAAT |
| | AAAGGTGGTAGTATTGTCATCATCGTGATAAACTACGGGAATATGGTCGTTAGTAGGTACGGTGACTTTA |
| | CACAACGCGATATATAACTTTCCTTTTGTACCATTTTTAACGTAGTTGGGACGTCCTGCAGGGTATTGTT |
| | TTGAAGAAATGATATCGAGAACAGATTTGATACGATATTTGTTGGATTCCTGATTATTCACTATAATATA |
| | ATCTAGACAGATAGATGATTCGATAAATAGAGAAGGTATATCGTTGGTAGGATAATACATCCCCATTCCA |
| | GTATTCTCGGATACTCTATTGATGACACTAGTTAAGAACATGTCTTCTATTCTAGAAAACGAAAACATCC |
| | TACATGGACTCATTAAAACTTCTAACGCTCCTGATTGTGTCTCGAATGCCTCGTACAAGGATTTCAAGGA |
| | TGCCATAGATTCTTTGACCAACGATTTAGAATTGCGTTTAGCATCTGATTTTTTTATTAAATCGAATGGT |
| | CGGCTCTCTGGTTTGCTACCCCAATGATAACAATAGTCTTGTAAAGATAAACCGCAAGAAAATTTATACG |
| | CATCCATCCAAATAACCCTAGCACCATCGGATGATATTAATGTATTATTATAGATTTTCCATCCACAATT |
| | ATTGGGCCAGTATACTGTTAGCAACGGTATATCGAATAGATTACTCATGTAACCTACTAGAATGATAGTT |
| | CGTGTACTAGTCATAATATCTTTAATCCAATCTAAGAAATTTAAAATTAGATTTTTTACACTGTTAAAGT |
| | TAACAAAGGTATTACCCGGATACGTGGATATCATATATGGCATTGGTCCATTATCAGTAATAGCTCCATA |
| | AACTGATACGGCGATGGTTTTTATATGTGTTTGATCTAACGAGGAAGAAATTCGCGCCCACAATTCATCT |
| | CTAGATATGTATTTAATATCAAACGGTAACACATCAATTTCGGGACGCGTATATGTTTCTAAATTTTTAA |
| | TCCAAATATAATGATGACCTATATGCCCTATTATCATACTGTCAACTATAGTACACCTAGAGAACTTACG |
| | ATACATCTGTTTCCTATAATCGTTAAATTTTACAAATCTATAACATGCTAAACCTTTTGACGACAGCCAT |
| | TCATTAATTTCTGATATGGAATCTGTATTCTCGATACCGTATTGTTCTAAAGCCAGTGCTATATCTCCCT |
| | GTTCGTGGGAACGCTTTCGTATAATATCGATCAACGGATAATCTGAAGTTTTTGGAGAATAATATGACTC |
| | ATGATCTATTTCGTCCATAAACAATCTAGACATAGGAATTGGAGGCGATGATCTTAATTTTGTGCAATGA |
| | GTCGTCAATCCTATAACTTCTAATCTTGTAATATTCATCATCGACATAATACTATCTATGTTATCATCGT |
| | ATATTAGTATACCACGGCCTTCTTCATTTCGTGCCAAAATAATATACAGTCTTAAATAATTACGCAATAT |
| | CTCAATAGTTTCATAATTGTTAGCTGTTTTCATCAAGATTTGTACCCTGTTTAACATGATGGCGTTCTAT |
| | ACGTCTCTATTTTCTTTTTTTTAAATTTTTAACGATTTACTGTGGCTAGATACCCAATCTCTCTCAAATA |
| | TTTTTTTAGCCTCGCTTACAAGCTGTTTATCTATACTATTAAAACTGACGAATCCGTGATTTTGGTAATG |
| | GGTTCCGTCGAAATTTGCCGAAGTGATATGAACATATTCGTCGTCGACTATCAACAATTTTGTATTATTC |
| | TGAATAGTGAAAACCTTCACAGATAGATCATTTTGAACACACAACGCGTCTAGACTTCTGGCGGTTGCCA |
| | TAGAATATACGTCGTTCTTATCCCAATTACCAACTAGAAGTCTGATCTTAACTCCTCTATTAATGGCTGC |
| | TTCTATAATGGAGTTGTAAATGTCGGGCCAATAGTAGCTATTACCGTCGACACGTGTAGTGGGAACTATG |
| | GCCAAATGTTCAATATCTATACTAGTCTTAGCCGACTTGAGTTTATCAATAACTACATCGGTATCTAGAT |
| | CTCTAGAATATCCCAATAGGTGTTCCGGAGAATCAGTAAAGAACACTCCACCTATAGGATTCTTAATATG |
| | ATACGCAGTGCTAACTGGCAAACAACAAGCCGCAGAGCATAAATTCAACCATGAATTTTTTGCGCTATTA |
| | AAGGCTTTAAAAGTATCAAATCTTCTACGAAGATCTGTGGCCAGCGGGGGATAATCAGAATATACACCTA |
| | ACGTTTTAATCGTATGTATAGATCCTCCAGTAAATGACGCGTTTCCTACATAACATCTTTCATCATCTGA |
| | CACCCAAAAACAACCGAGTAGTAGTCCCACATTATTTTTTTTATCTATATTAACGGTTATAAAATTTATA |
| | TCCGGGCAGTGACTTTGTAGCTCTCCCAGATTTCTTTTCCCTCGTTCATCTAGCAAAACTATTATTTTAA |
| | TCCCTTTTTCAGATGCCTCTTTTAGTTTATCAAAAATAAGCGCTCCCCTAGTCGTACTCAGAGGATTACA |
| | ACAAAAAGATGCTATGTATATATATTTCTTAGCTAGAGTGATAATTTCGTTAAAACATTCAAATGTTGTT |
| | AAATGATCGGATCTAAAATCCATATTTTCTGGTAGTGTTTCTACCAGCCTACATTTTGCTCCCGCAGGTA |
| | CCGATGCAAATGGCCACATTTAGTTAACATAAAAACTTATACATCCTGTTCTATCAACGATTCTAGAATA |
| | TCATCGGCTATATCGCTAAAATTTTCATCAAAGTCGACATCACAACCTAACTCAGTCAATATATTAAGAA |
| | GTTCCATGATGTCATCTTCGTCTATTTCTATATCCGTATCCATTGTAGATTGTTGACCGATTATCGAGTT |
| | TAAATCATTACTAATACTCAATCCTTCAGAATACAATCTGTGTTTCATTGTAAATTTATAGGCGGTGTAT |
| | TTAAGTTGGTAGATTTTCAATTATGTATCAATATAGCAACAGTAGTTCTTGCTCCTCCTTGATTCTAGCA |
| | TCCTCTTCATTATTTTCTTCTACGTACATAAGCATGTCCAATACGTTAGACAACACACCGACGATGGCGG |
| | CCGCCACAGACACGAATATGACTAGACCGATGACCATTTAAAAACCCCTCTCTAGCTTTCACTTAAACTG |
| | TATCGATTATTCTTTTAGAACATGTATAATATAAAAACATTATTCTATTTCGAATTTAGGCTTCCAAAAA |
| | TTTTTCATCCGTAAACCGATAATAATATATATAGACTTGTTAATAGTCGGAATAAATAGATTAATGCTTA |
| | AACTATCATCATCTCCACGATTAGAGATACAATATTTACATTTTTTTTGCTGTTTCGAAACTTTATCAAT |
| | ACACGTTAATACAAACCCAGGAAGGAGATATTGAAACTGAGGCTGTTGAAAATGAAACGGTGAATACAAT |
| | AATTCAGATAATGTAAAATCATGATTCCGTATTCTGATGATATTAGAACTGCTAATGGATGTCGATGGTA |
| | TGTATCTAGGAGTATCTATTTTAACAAAGCATCGATTTGCTAATATACAATTATCCTTTTGATTAATTGT |
| | TATTTTATTCATATTCTTAAAAGGTTTCATATTTATCAATTCTTCTACATTAAAAATTTCCATTTTTAAT |
| | TTATGTAGCCCCCGCAATACTCCTCATTACGTTTCATTTTTTGTCTATAATATCCATTTTGTTCATCTCG |
| | GTACATAGATTATCCAATTGAGAAGCGCATTTAGTAGTTTTGTACATTTTAAGTTTATTGACGAATCGTC |
| | GAAAACTAGTTATAGTTAACATTTTATTATTTGATACCCTGATATTAATACCCCTGCCGTTACTATTATT |
| | TATAACTGATGTAATCCACGTAACATTAGAATTAATTATCGATAGTAATGCATCGACGCTTCCAAAATTG |
| | TCTATTATAAACTCACCGATAATTTTTTTATTGCATGTTTTCATATTCATTAGGATTATCAAATCTTTAA |
| | TCTTACTACGATTGTATGCGTTGATATTGCAAGACGTCATTCTAAAAGACGGAGGATCTCCATCAAATGC |
| | CAGACAATCACGTACAAAGTACATGGAAATAGGTTTTGTTCTATTGCGCATCATAGATTTATATAGAACA |
| | CCCGTAGAAATACTAATTTGTTTTACTCTATAAAATACTAATGCATCTATTTCATCGTTTTGTATAACGT |
| | CTTTCCAAGTGTCAAATTCCAAATTTTTTTCATTGATAGTACCAAATTCTTCTATCTCTTTAACTACTTG |
| | CATAGATAGGTAATTACAGTGATGCCTACATGCCGTTTTTTGAAACTGAATAGATGCGTCTAGAAGCGAT |
| | GCTACGCTAGTCACAATCACCACTTTCATATTTAGAATATATGTATGTAAAAATATAGTAGAATTTCATT |
| | TTGTTTTTTTCTATGCTATAAATGAATTCTCATTTTGCATCTGCTCATACTCCGTTTTATATCAATACCA |
| | AAGAAGGAAGATATCTGGTTCTAAAAGCCGTTAAAGTATGCGATGTTAGAACTGTAGAATGCGAAGGAAG |
| | TAAAGCTTCCTGCGTACTCAAAGTAGATAAACCCTCATCGCCCGCGTGTGAGAGAAGACCTTCGTCCCCG |
| | TCCAGATGCGAGAGAATGAATAACCCTGGAAAACAAGTTCCGTTTATGAGGACGGACATGCTACAAAATA |
| | TGTTCGCGGCTAATCGCGACAACGTGGCGTCGAGACTTTTGAACTAAAATACAATTATATCCTTTTCGAT |
| | ATTAATAAATCCGTGTCGTCCAGGTTTTTTATCTCTTTCAGTATGTGAATAGATAGGTATTTTATCTCTA |
| | TTCATCATCGAATTTAAGAGATCCGATAAACATTGTTTGTATTCTCCAGATGTCAGCATCTGATACAACA |
| | ATATATGTGCACATAAACCTCTGGCACTTATTTCATGTACCTTCCCCTTATCACTAAGGAGAATAGTATT |
| | TGAGAAATATGTATACATGATATTATCATGAATTAGATATACAGAATTTGTAACACTCTCGAAATCACAC |
| | GATGTGTCGGCGTTAAGATCTAATATATCACTCGATAACACATTTTCATCTAGATACACTAGACATTTTT |
| | TAAAGCTAAAATAGTCTTTAGTAGTGACAGTAACTATGCGATTATTTTCATCGATGATACATTTCATCGG |
| | CATATTATTACGCTTACCATCAAAGACTATACCATGTGTATATCTAACGTATTCTAGCATGGTTGCCATA |
| | CGCGCATTAAACTTTTCAGGATCTTTGGATAGATCTTCCAATCTATCTATTTGAGAAAACATTTTTATCA |
| | TGTTCAATAGTTGAAACGTCGGATCCACTATATAGATATTATCTATAAAGATTTTAGGAACTACGTTCAT |
| | GGTATCCTGGCGAATATTAAAACTATCAATGATATGATTATCGTTTTCATCTTTTATCACCATATAGTTT |
| | CTAAGATATGGGATTTTACTTAATATAATATTATTTCCCGTGATAAATTTTATTAGAAAGGCCAAATCTA |
| | TAAGAAAAGTTCTAGAATTAGTCTGAAGAATATCTATATCGCCGTACCGTATATTTGGATTAATTAGATA |
| | TAGAGAATATGATCCGTAACATATACAACTTTTATTATGGCGTCTAAGATATTCTTCCATCAACTTATTA |
| | ACATTTTTGACTAGGGAAGATACATTATGACGTCCCATTACTTTTGCCTTGTCTATTACTGCGACGTTCA |
| | TAGAATTTAGCATATCTCTTGCCAATTCTTCCATTGATGTTACATTATAAGAAATTTTAGATGAAATTAC |
| | ATTTGGAGCTTTAATAGTAAGAACTCCTAATATGTCCGTGTATGTGGTCACTAATACAGATTGTAGTTCT |
| | ATAATCGTAAATAATTTACCTATATTATATGTTTGAGTCTGTTTAGAAAAGTAGCTAAGTATACGATCTT |
| | TTATTTCTGATGCAGATGTATTAACATCGGAAAAAAATCTTTTTTTATTCTTTTTTACTAAAGATACAAA |
| | TATGTCTTTGTTAAAAACAGTTATTTTTTGAATATTTCTAGCTTGTAATTTTAACATATGATATTCGTTC |
| | ACACTAGGTACTCTGCCTAAATAGGTTTCTATAATCTTTAATGTAATATTAGGAAGAGTATTCTGATCAG |
| | GATTCCTATTCATTTTGAGGATTTAAAACTCTGATTATTGTCTAATATGGTCTCTACGCAAACTTTTTCA |
| | CAGAGCGATAGAGTTTTTGATAACTCGTTTTTCTTAAGAAATATAAAACTACTGTCTCCAGAGCTCGCTC |
| | TATCTTTTATTTTATTTAATTCGATACAAACTCCTGATACTGGTTCAGAAAGTAATTCATTAATTTTCAG |
| | TCCTTTATAGAAGATATTTAATATAGATAATACAAAATTTTCAGTTCTTGATATCGATCTGATTGATCCT |
| | AGAACTAGATATATTAATAACGTGCTCATTAGGCAGTTTATGGCAGCTTGATAATTAGATATAGTATATT |
| | CCAGTTCATATTTATTAGATACCGCATTGCCCAGATTTTGATATTCTATGAATTCCTCTGAAAATAAATC |
| | CAAAATAACTAAACATTCTATTTTTTGTGGATTAGTGTACTCTCTTCCCTCTATCATGTTCACTACTGGT |
| | GTCCACGATGATAAATATCTAGAGGGAATATAATATAGTCCATAGGATGCCAATCTAGCAATGTCGAATA |
| | ACTGTAATTTGATTCTTCGTTCTTCATTATGAATTGATTCTTGAGGTATAAACCTAACACAAATTATATT |
| | ATTAGACTTTTCGTATGTAATGTCTTTCATGTTATAAGTTTTTAATCCTGGAATAGAATCTATTTTAATG |
| | AGGCTTTTAAACGCAGAGTTCTCCAACGAGTCAAAGCATAATACTCTGTTGTTTTTCTTATATACGATGT |
| | TACGATTTTCTTCTTTGAATGGAATAGGTTTTTGAATTAGTTTATAATTACAACATAATAGATAAGGAAG |
| | TGTGCAAATAGTACGCGGAAAAAACATAATAGCTCCCCTGTTTTCATCCATGGTTTTAAGTAAATGATCA |
| | CTGGCTTCTTTAGTCAATGGATATTCGAACATTAACCGTTTCATCATCATTGGACAGAATCCATATTTCT |
| | TAATGTAAAGAGTGATCAAATCATTGTGTTTATTGTACCATCTTGTTGTAAATGTGTATTCGGTTATCGG |
| | ATCTGCTCCTTTTTCTATTAAAGTATCGATGTCGATCTCGTCTAAGAATTCAACTATATCGACATATTTC |
| | ATTTGTATACACATAACCATTACTAACGTAGAATGTATAGGAAGAGATGTAACGGGAACAGGGTTTGTTG |
| | ATTCGCAAACTATTCTAATACATAATTCTTCTGTTAATACGTCTTGCACGTAATCTATTATAGATGCCAA |
| | GATATCTATATAATTATTTTGTAAGATGATGTTAACTATGTGATCTATATAAGTAGTGTAATAATTCATG |
| | TATTTCGATATATGTTCCAACTCTGTCTTTGTGATGTCTAGTTTCGTAATATCTATAGCATCCTCAAAAA |
| | ATATATTCGCATATATTCCCAAGTCTTCAGTTCTATCTTCTAAAAAATCTTCAACGTATGGAATATAATA |
| | ATCTATTTTACCTCTTCTGATATCATTAATGATATAGTTTTTGACACTATCTTCTGTCAATTGATTCTTA |
| | TTCACTATATCTAAGAAACGGATAGCGTCCCTAGGACGAACTACTGCCATTAATATCTCTATTATAGCTT |
| | CTGGACATAATTCATCTATTATACCAGAATTAATGGGAACTATTCCGTATCTATCTAACATAGTTTTAAG |
| | AAAGTCAGAATCTAAGACCTGATGTTCATATATTGGTTCATACATGAAATGATCTCTATTGATGATAGTG |
| | ACTATTTCATTCTCTGAAAATTGGTAACTCATTCTATATATGCTTTCCTTGTTGATGAAGGATAGAATAT |
| | ACTCAATAGAATTTGTACCAACAAACTGTTCTCTTATGAATCGTATATCATCATCTGAAATAATCATGTA |
| | AGGCATACATTTAACAATTAGAGACTTGTCTCCTGTTATCAATATACTATTCTTGTGATAATTTATGTGT |
| | GAGGCAAATTTGTCCACGTTCTTTAATTTTGTTATAGTAGATATCAAATCCAATGGAGCTACAGTTCTTG |
| | GCTTAAACAGATATAGTTTTTCTGGAACGAATTCTACAACATTATTATAAAGGACTTTGGGTAGATAAGT |
| | GGGATGAAATCCTATTTTAATTAATGCGATAGCCTTGTCCTCGTGCAGATATCCAAACGCTTTTGTGATA |
| | GTATGGCATTCATTGTCTAGAAACGCTCTACGAATATCTGTGACAGATATCATCTTTAGAGAATATACTA |
| | GTCGCGTTAATAGTACTACAATTTGTATTTTTTAATCTATCTCAATAAAAAAATTAATATGTATGATTCA |
| | ATGTATAACTAAACTACTAACTGTTATTGATAACTAGAATCAGAATCTAATGATGACGTAACCAAGAAGT |
| | TTATCTACTGCCAATTTAGCTGCATTATTTTTAGCATCTCGTTTAGATTTTCCATCTGCCTTATCGAATA |
| | CTCTTCCGTCGATGTCTACACAGGCATAAAATGTAGGAGAGTTACTAGGCCCCACTGATTCAATACGAAA |
| | AGACCAATCTCTCCTAGTAATTTGGCAGTACTCATTAATAACGGTGACAGGGTTAGCACCTTTCCAATCA |
| | ATAATTTTTTTAGCCGGAATAACATCATCAAAAGACTTATGATCCTCTCTCATTGATTTTTCGCGGGATA |
| | CATCATCTATTATGACGTCAGCCATAGCATCAGCATCCGGCTTATCCGCCTCCGTTGTCATAAACCAACG |
| | AGGAGGAATATCGTCGGAGCTGTACACCATAGCACTACGTTGAAGATCGTACAGAGCTTTATTAACTTCT |
| | CGCTTCTCCATATTAAGTTGTCTAGTTAGTTGTGCAGCAGTAGCTCCTTCGATTCCAATGGTTTTAATAG |
| | CCTCACACACAATCTCTGCGTTAGAACGCTCGTCGATATAGATTTTAGACATTTTTAGAGAGAACTAACG |
| | CAATCAGTAATAAAACTAATTTATTTTATCATTTTTTTTATTCATCATCCTCTGGTGGTTCGTCGTTTCT |
| | ATCGAATGTAGCTCTGATTAACCCGTCATCTATAGGTGATGCTGGTTCTGGAGATTCTGGAGGAGATGGA |
| | TTATTATCTGGAAGAATCTCTGTTATTTCCTTGTTTTCATGTATCGATTGCGTTGTAACATTAAGATTGC |
| | GAAATGCTCTAAATTTGGGAGGCTTAAAGTGTTGTTTGCAATCTCTACACGCGTGTCTAACTAGTGGAGG |
| | TTCGTCAGCGGCTCTAGTTTGAATCATCATCGGCGTAGTATTCCTACTTTTACAGTTAGGACACGGTGTA |
| | TTGTATTTCTCGTCGAGAACGTTAAAATAATCGTTGTAACTCACATCCTTTATTTTATCTATATTGTATT |
| | CTACTCCTTTCTTAATGCATTTTATACCGAATAAGAGATAGCGAAGGAATTCTTTTTCGGTGCCGCTAGT |
| | ACCCTTAATCATATCACATAGTGTTTTATATTCCAAATTTGTGGCAATAGACGGTTTATTTCTATACGAT |
| | AGTTTGTTTCTGGAATCCTTTGAGTATTCTATACCAATATTATTCTTTGATTCGAATTTAGTTTCTTCGA |
| | TATTAGATTTTGTATTACCTATATTCTTGATGTAGTACTTTGATGATTTTTCCATGGCCCATTCTATTAA |
| | GTCTTCCAAGTTGGCATCATCCACATATTGTGATAGTAATTCTCGGATATCAGTAGCGGTTACCGCCATT |
| | GATGTTTGTTCATTGGATGAGTAACTACTAATGTATACATTTTCCATTTATAACACTTATGTATTAACTT |
| | TGTTCATTTATATTTTTTCATTATTATGTTGATATTAACAAAAGTGAATATATATATGTTAATAATTGTA |
| | TTGTGGTTATACGGCTACAATTTTATAATTAGTGAAAGTCAGTGTCCGATGATCAATGACGATAGCTTTA |
| | CTCTGAAAAGAAAGTATCAAATCGATAGTGCGGAGTCAACAATAAAAATGGATAAGAAGAGGACAAAGTT |
| | TCAGAATAGAGCCAAAATGGTAAAAGAAATAAATCAGACAATAAGAGCAGCACAAACTCATTACGAGACA |
| | TTGAAACTAGGATACATAAAATTTAAGAGAATGATTAGGACTACTACTCTAGAAGATATAGCACCATCTA |
| | TTCCAAATAATCAGAAAACTTATAAACTATTCTCGGACATTTCAGCCATCGGCAAAGCATCACGGAATCC |
| | AAGTAAGATGGTATATGCTCTGCTGCTTTACATGTTTCCCAATTTGTTTGGAGATGATCATAGATTCATT |
| | CGTTATAGAATGCATCCAATGAGTAAAATCAAACACAAGATCTTCTCTCCTTTCAAACTTAATCTTATTA |
| | GAATATTAGTGGAAGAAAGATTCTATAATAATGAATGCAGATCTAATAAATGGAGAATAATTGGAACACA |
| | AGTTGATAAAATGTTGATAGCTGAATCTGATAAATATACAATAGATGCAAGGTATAACCTAAAACCCATG |
| | TATAGAATCAAGGGAAAATCTGAAGAAGATACCCTCTTTATCAAACAGATGGTAGAACAATGTGTGACAT |
| | CCCAGGAATTGGTGGAAAAAGTGTTGAAGATACTGTTTAGAGATTTGTTCAAGAGTGGAGAATACAAAGC |
| | GTACAGATACGATGATGATGTAGAAAATGGATTTATTGGATTGGATACACTAAAATTAAACATTGTTCAT |
| | GATATAGTTGAACCATGTATGCCTGTTCGTAGGCCAGTGGCTAAGATACTGTGTAAAGAAATGGTAAATA |
| | AATACTTTGAGAATCCGCTACATATTATTGGTAAAAATCTTCAAGAGTGCATTGACTTTGTTAGTGAATA |
| | GGCATTTCATCTTTCTCCAATACTAATTCAAATTGTTAAATTAATAATGGATAGTATAAATAGTTATTAG |
| | TGATAAAATAGTAAAAATAATTATTAGAATAAGAGTGTAGTATCATAGATAACTCTCTTCTATAAAAATG |
| | GATTTTATTCGTAGAAAGTATCTTATATACACAGTAGAAAATAATATAGATTTTTTAAAGGATGATACAT |
| | TAAGTAAAGTAAACAATTTTACCCTCAATCATGTACTAGCTCTCAAGTATCTAGTTAGCAATTTTCCTCA |
| | ACATGTTATTACTAAGGATGTATTAGCTAATACCAATTTTTTTGTTTTCATACATATGGTACGATGTTGT |
| | AAAGTGTACGAAGCGGTTTTACGACACGCATTTGATGCACCCACGTTGTACGTTAAAGCATTGACTAAGA |
| | ATTATTTATCGTTTAGTAACACAATACAATCGTACAAGGAAACCGTGCATAAACTAACACAAGATGAAAA |
| | ATTTTTAGAGGTTGCCAAATACATGGACGAATTAGGAGAACTTATAGGCGTAAATTATGACTTAGTTCTT |
| | AATCCATTATTTCACGGAGGGGAACCCATCAAAGATATGGAAATCATTTTTTTAAAACTGTTTAAGAAAA |
| | CAGACTTCAAAGTTGTTAAAAAATTAAGTGTTATAAGATTACTTATTTGGGCTTACCTAAGCAAGAAAGA |
| | TACAGGCATAGAGTTTGCGGATAATGATAGACAAGATATATATACTCTATTTCAACAAACTGGTAGAATC |
| | GTCCATAGCAATCTAACAGAAACGTTTAGAGATTATATCTTTCCCGGAGATAAGACTAGCTATTGGGTGT |
| | GGTTAAACGAAAGTATAGCTAATGATGCGGATATTGTTCTTAATAGACACGCCATTACCATGTATGATAA |
| | AATTCTTAGTTATATATACTCTGAGATAAAACAAGGACGCGTTAATAAAAACATGCTTAAGTTAGTTTAT |
| | ATCTTTGAGCCTGAAAAAGATATCAGAGAACTTCTGCTAGAAATCATATATGATATTCCTGGAGATATCC |
| | TATCTATTATTGATGCAAAAAACGACGATTGGAAAAAATATTTTATTAGTTTTTATAAAGCTAATTTTAT |
| | TAACGGTAATACATTTATTAGTGATAGAACGTTTAACGAGGACTTATTCAGAGTTGTTGTTCAAATAGAT |
| | CCCGAATATTTCGATAATGAACGAATTATGTCTTTATTCTCTACGAGTGCTGCGGACATTAAACGATTTG |
| | ATGAGTTAGATATTAATAACAGTTATATATCTAATATAATTTATGAGGTGAACGATATCACATTAGATAC |
| | AATGGATGATATGAAGAAGTGTCAAATCTTTAACGAGGATACGTCGTATTATGTTAAGGAATACAATACA |
| | TACCTGTTTTTGCACGAGTCGGATCCCATGGTCATAGAGAACGGAATACTAAAGAAACTGTCATCTATAA |
| | AATCCAAGAGTAGACGGCTGAACTTGTTTAGCAAAAACATTTTAAAATATTATTTAGACGGACAATTGGC |
| | TCGTCTAGGTCTTGTGTTAGATGATTATAAAGGAGACTTGTTAGTTAAAATGATAAACCATCTTAAGTCT |
| | GTGGAGGATGTATCCGCATTCGTTCGATTTTCTACAGATAAAAACCCTAGTATTCTTCCATCGCTAATCA |
| | AAACTATTTTAGCTAGTTATAATATTTCCATCATCGTCTTATTTCAAAGGTTTTTAAGAGATAATCTATA |
| | TCATGTAGAAGAATTCTTGGATAAAAGCATCCATCTAACCAAGACGGATAAGAAATATATACTTCAATTG |
| | ATAAGACACGGTAGATCATAGAACAGACCAAATATATTATTAATAATTTGTATATACATAGATATAATTA |
| | TCACATATTAAAAATTCACACATTTTTGATAAATGGGAACTGCTGCAACAATTCAGACTCCCACCAAATT |
| | AATGAATAAAGAAAATGCAGAAATGATTTTGGAAAAAATTGTTGATCATATAGTTATGTATATTAGTGAC |
| | GAATCAAGTGATTCAGAAAATAATCCTGAATATATTGATTTTCGTAACAGATACGAAGACTATAGATCTC |
| | TCATTATAAAAAGTGATCACGAGTTTGTAAAGCTATGTAAAAATCATGCAGAGAAAAGTTCTCCAGAAAC |
| | GCAACAAATGATTATCAAACACATATACGAACAATATCTTATTCCAGTATCTGAAGTACTATTAAAACCT |
| | ATAATGTCCATGGGTGACATAATTACATATAACGGATGTAAAGACAATGAATGGATGCTAGAACAACTCT |
| | CTACCCTAAACTTTAACAATCTCCGCACATGGAACTCATGTAGCATAGGCAATGTAACGCGTCTGTTTTA |
| | TACATTTTTTAGTTATCTGATGAAAGATAAACTAAATATATAAGTATAATCCCATTCTAATACTTTAACC |
| | TGATGTATTAGCATCTTATTAGAATATTAACCTAACTAAAAGACATAACATAAAAACTCATTACATAGTT |
| | GATAAAAAGCGGTAGGATATAAATATTATGGCTGCCACCGTTCCGCGTTTTGACGACGTGTACAAAAATG |
| | CACAAAGAAGAATTCTAGATCAAGAAACATTTTTTAGTAGAGGTCTAAGTAGACCGTTAATGAAAAACAC |
| | ATATCTATTTGATAATTACGCGTATGGATGGATACCAGAAACTGCAATTTGGAGTAGTAGATACGCAAAC |
| | TTAGATGCAAGTGACTATTATCCCATTTCGTTGGGATTACTTAAAAAGTTCGAGTTTCTCATGTCTCTAT |
| | ATAAAGGTCCTATTCCAGTATACGAAGAAAAAGTAAATACTGAATTCATTGCTAATGGATCGTTCTCTGG |
| | TAGATACGTATCATATCTTCGAAAGTTTTCTGCTCTTCCAACAAACGAGTTTATTAGTTTTTTGTTACTG |
| | ACTTCCATTCCAATCTATAATATCTTGTTCTGGTTTAAAAATACTCAGTTTGATATTACTAAACACACAT |
| | TATTCAGATACGTCTATACAGATAATGCCAAACACCTGGCGTTGGCTAGGTATATGCATCAAACAGGAGA |
| | CTATAAGCCTTTGTTTAGTCGTCTCAAAGAGAATTATATATTTACCGGTCCCGTTCCAATAGGTATCAAA |
| | GATATAAATCACCCTAATCTTAGTAGAGCAAGAAGTCCATCCGATTATGAGACATTAGCTAATATTAGTA |
| | CTATATTGTACTTTACCAAGTATGATCCGGTATTAATGTTTTTATTGTTTTACGTACCTGGGTATTCAAT |
| | TACTACAAAAATTACTCCAGCCGTAGAATATCTAATGGATAAACTGAATCTAACAAAGAGCGACGTACAA |
| | CTGTTGTAAATTATTTTATGCTTCGTAAAATGTAGGTTTTGAACCAAACATTCTTTCAAAGAATGAGATG |
| | CATAAAACTTTATTATCCAATAGATTGACTATTTCGGACGTCAATCGTTTAAAGTAAACTTCGTAAAATA |
| | TTCTTTGATCACTGCCGAGTTTAAAACTTCTATCGATAATTGTTTCATATGTTTTAATATTTACAAGTTT |
| | TTTGGTCCATGGTACATTAGCCGGACAAATATATGCAAAATAATATCGTTCTCCAAGTTCTATAGTTTCT |
| | GGATTATTTTTATTATATTCAGTAACCAAATACATATTAGGGTTATCTGCGGATTTATAATTTGAGTGAT |
| | GCATTCGACTCAACATAAATAATTCTAGAGGAGACGATCTACTATCAAATTCGGATCGTAAATCTGTTTC |
| | TAAAGAACGGAGAATATCTATACATACCTGATTAGAATTCATCCGTCCTTCAGACAACATCTCAGACAGT |
| | CTGGTCTTGTATGTCTTAATCATATTCTTATGAAACTTGGAAACATCTCTTCTAGTTTCACTAGTACCTT |
| | TATTAATTCTCTCAGGTACAGATTTTGAATTCGACGATGCCGAGTATTTCATCGTTGTATATTTCTTCTT |
| | CGATTGCATAATCAGATTCTTATATACCGCCTCAAACTCTATTTTAAAATTATTAAACAATACTCTATTA |
| | TTAATCAGTCGTTCTAACTCCTTTGCTATTTCTATGGACTTATCTACATCTTGACTGTCTATCTCTGTAA |
| | ACACGGAGTCGGTATCTCCATACACGCTACGAAAACGAAATCTGTAATCTATAGGCAACGATGTTTTCAC |
| | AATCGGATTAATATCTCTATCGTCCATATAAAATGGATTACTTAATGGATTGGCAAACCGTAACATACCG |
| | TTAGATAACTCTGCTCCATTTAGTACCGATTCTAGATACAAGATCATTCTACGTCCTATGGATGTGCAAC |
| | TCTTAGCCGAAGCGTATGAGTATAGAGCACTATTTCTAAATCCCATCAGACCATATACTGAGTTGGCTAC |
| | TATCTTGTACGTATATTGCATGGAATCATAGATGGCCTTTTCAGTTGAACTGGTAGCCTGTTTTAACATC |
| | TTTTTATATCTGGCTCTCTCTGCCAAAAATGTTCTTAATAGTCTAGGAATGGTTCCTTCTATCGATCTAT |
| | CGAAAATTGCTATTTCAGAGATGAGGTTCGGTAGTCTAGGTTCACAATGAACCGTAATATATCTAGGAGG |
| | TGGATATTTCTGAAGCAAGAGCTGATTATTTATTTCTTCTTCCAATCTATTGGTACTAACAACGACACCG |
| | ACTAATGTTTCCGGAGATAGATTTCCAAAGATACACACATTAGGATACAGACTGTTATAATCAAAGATTA |
| | ATACATTATTACTAAACATTTTTTGTTTTGGAGCAAATACCTTACCGCCTTCATAAGGAAACTTTTGTTT |
| | TGTTTCTGATCTAACTAAGATAGTTTTAGTTTCCAACAATAGCTTTAACAGTGGACCCTTGATGACTGTA |
| | CTCGCTCTATATTCGAATACCATGGATTGAGGAAGCACATATGTTGACGCACCCGCGTCTGTTTTTGTTT |
| | CTACTCCATAATACTCCCACAAATACTGACACAAACAAGCATCATGAATACAGTATCTAGCCATATCTAA |
| | AGCTATGTTTAGATTATAATCCTTATACATCTGAGCTAAATCAACGTCATCCTTTCCGAAAGATAATTTA |
| | TATGTATCATTAGGTAAAGTAGGACATAATAGTACGACTTTAAATCCATTTTCCCAAATATCTTTACGAA |
| | TTACTTTACATATAATATCCTCATCAACAGTCACATAATTACCTGTGGTTAAAACCTTTGCAAATGCAGC |
| | GGCTTTGCCTTTCGCGTCCGTAGTATCGTCACCGATGAACGTCATTTCTCTAACTCCTCTATTTAATACT |
| | TTACCCATGCAACTGAACGCGTTCTTGGATATAGAATCCAATTTGTACGAATCCAATTTTTCAGATTTTT |
| | GAATGAATGAATATAGATCGAAAAATATAGTTCCATTATTGTTATTAACGTGAAACGTAGTATTGGCCAT |
| | GCCGCCTACTCCCTTATGACTAGACTGATTTCTCTCATAAATACAGAGATGTACAGCTTCCTTTTTGTCC |
| | GGAGATCTAAAGATAATCTTCTCTCCTGTTAATAACTCTAGACGATTAGTAATATATCTCAGATCAAAGT |
| | TATGTCCGTTAAAGGTAACGACATAGTCGAACGTTAGTTCCAACAATTGTTTAGCTATTCGTAACAAAAC |
| | TATTTCAGAACATAAAACTAGTTCTCGTTCGTAATCCATTTCCATTAGTGACTGTATCCTCAAACATCCT |
| | CTATCGACGGCTTCTTGTATTTCCTGTTCCGTTAACATCTCTTCATTAATGAGCGTAAACAATAATCGTT |
| | TACCACTTAAATCGATATAACAGTAACTTGTATGCGAGATTGGGTTAATAAATACAGAAGGAAACTTCTT |
| | ATCGAAGTGACACTCTATATCTAGAAATAAGTACGATCTTGGGATATCGAATCTAGGTATTTTTTTAGCG |
| | AAACAGTTACGTGGATCGTCACAATGATAACATCCATTGTTAATCTTTGTCAAATATTGCTCGTCCAACG |
| | AGTAACATCCGTCTGGAGATATCCCGTTAGAAATATAAAACCAACTAATATTGAGAAATTCATCCATGGT |
| | GGCATTTTGTATGCTGCGTTTCTTTGGCTCTTCTATCAACCACATATCTGCGACGGAGCATTTTCTATCT |
| | TTAATATCTAGATTATAACTTATTGTCTCGTCAATGTCTATAGTTCTCATCTTTCCCAACGGCCTCGCAT |
| | TAAATGGAGGAGGAGACAATGACTGATATATTTCGTCCGTCACTACGTAATAAAAGTAATGAGGAAATCG |
| | TATAAATACGGTCTCACCATTTCGACATCTGGATTTCAGATATAAAAATCTGTTTTCACCGTGACTTTCA |
| | AACCAATTAATGCACCGAACATCCATTTATAGAATTTAGAAATATATTTTCATTTAAATGAATCCCAAAC |
| | ATTGGGGAAGAGCCGTATGGACCATTATTTTTATAGTACTTTCGCAAGCGGGTTTAGACGGCAACATAGA |
| | AGCGTGTAAACGAAAACTATATACTATAGTTAGCACTCTTCCATGTCCTGCATGTAGACGGCACGCGACT |
| | ATCGCTATAGAGGACAATAATGTCATGTCTAGCGATGATCTGAATTATATTTATTATTTTTTCATCAGAT |
| | TATTTAACAATTTGGCATCTGATCCCAAATACGCGATCGATGTGACAAAGGTTAACCCTTTATAAACTTA |
| | ACCCATTATAAAACTTATGATTAGTCACGACTGAAATAACCGCGTGATTATTTTTTGGTATAATTCTACA |
| | CGGCATGGTTTCTGTGACTATGAATTCAACCCCCGTTACATTAGTGAAATCTTTAACAAACAGCAAGGGT |
| | TCGTCAAAGACATAAAACTCATTGTTTACAATCGAAATAGACCCCCTATCACACTTAAAATAAAAAATAT |
| | CCTTATCCTTTACCACCAAATAAAATTCTGATTGGTCAATGTGAATGTATTCACTTAACAGTTCCACAAA |
| | TTTATTTATTAACTCCGAGGCACATACATCGTCGGTATTTTTTATGGCAAACTTTACTCTTCCAGCATCC |
| | GTTTCTAAAAAAATATTAACGAGTTCCATTTATATCATCCAATATTATTGAAATGACGTTGATGGACAAA |
| | TGATACAAATAAGAAGGTACGGTACCTTTGTCCACCATCTCCTCCAATTCATGCTCTATTTTGTCATTAA |
| | CTTTAATGTATGAAAACAGTACGCCACATGCTTCCATGACAGTGTGTAACACTTTGGATACAAAATGTTT |
| | GACATTAGTATAATTGTTCAAGACTGTCAATCTATAATAGATAGTAGCTATAATATATTCTATGATGGTA |
| | TTGAAGAAGATGACAACCTTGGCATATTGATCATTTAACACAGACATGGTATCAACAGATAGCTTGAATG |
| | AAAGAGAATCAGTAATTGGAATAAGCGTCTTCTCGATGGAGTGTCCGTATACCAACATGTCTGATATTTT |
| | GATGTATTCCATTAAATTATTTAGTTTTTTCTTTTTATTCTCGTTAAACAGCATTTCTGTCAACGGACCC |
| | CAACATCGTTGACCGATTAAGTTTTGATTGATTTTTCCGTGTAAGGCGTATCTAGTCAGATCGTATAGCC |
| | TATCCAATAATCCATCGTCTGTGTGTAGATCACATCGTACACTTTTTAATTCTCTATAGAAGAGCGACAG |
| | ACATCTGGAGCAATTACAGACAGCAATTTCTTTATTCTCTACAGATGTAAGATACTTGAAGACATTCCTA |
| | TGATGATGCAGAATTTTGGATAACACGGTATTGATGGTATCTGTTACCATAATTCCTTTGATGGCTGATA |
| | GTGTCAGAGCACAAGATTTCCAATCTTTGACAATTTTTAGCACCATTATCTTTGTTTTGATATCTATATC |
| | AGACAGCATGGTGCGTCTGACAACACAAGGATTAAGACGGAAAGATGAAATGATTCTCTCAACATCTTCA |
| | ATGGATACCTTGCTATTTTTTCTGGCATTATCTATATGTGCGAGAATATCCTCTAGAGAATCAGTATCCT |
| | TTTTGATGATAGTGGATCTCAATGACATGGGACGTCTAAACCTTCTTATTCTATCACCAGATTGCATGGT |
| | GATTTGTCTTCTTTCTTTTATCATAATGTAATCTCTAAATTCATCGGCAAATTGTCTATATCTAAAATCA |
| | TAATATGAGATGTTTACCTCTACAAATATCTGTTCGTCCAATGTTAGAGTATTTACATCAGTTTTGTATT |
| | CCAAATTAAACATGGCAACGGATTTAATTTTATATTCCTCTATTAAGTCCTCGTCGATAATAACAGAATG |
| | TAGATAATCATTTAATCCATCGTACATGGTTGGAAGATGCTCGTTGACAAAATCTTTAATTGTCTTGATG |
| | AAGGTGGGACTATATCTAACATCTTGATTAATAAAATTTATAACATTGTCCATAGGATACTTTGTAACTA |
| | GTTTTATACACATCTCTTCATCGGTAAGTTTAGACAGAATATCGTGAACAGGTGGTATATTATATTCATC |
| | AGATATACGAAGAACAATGTCCAAATCTATATTGTTTAATATATTATATAGATGTAGTGTAGCTCCTACA |
| | GGAATATCTTTAACTAAGTCAATGATTTCATCAACCGTTAGATCTATTTTAAAGTTAATCATATAGGCAT |
| | TGATTTTTAAAAGGTATGTAGCCTTGACTACATTCTCATTAATTAACCATTCCAAGTCACTGTGTGTAAG |
| | AAGATTATATTCTATCATAAGCTTGACTACATTTGGTCCCGATACCATTAAAGAATTCTTATGATATAAG |
| | GAAACAGATTTTAGGTACTCATCTACTCTACAAGAATTTTGGAGAGCCTTAACGATATCAGTGACGTTTA |
| | TTATTTCAGGAGGAAAAAACCTAACATTGAGAATATCGGAATTAATAGCTTCCAGATACAGTGATTTTGG |
| | CAATAGTCCGTGTAATCCATAATCCAGTAACACGAGCTGGTGCTTGCTAGACACCTTTTCAATGTTTAAT |
| | TTTTTTGAAATAAGCTTTGATAAAGCCTTCCTCGCAAATTCCGGATACATGAACATGTCGGCGACATGAT |
| | TAAGTATTGTTTTTTCATTATTTTTATATTTTCTCAACAAGTTCTCAATACCCCAATAGATGATAGAATA |
| | TCACCCAATGCGTCCATGTTGTCTATTTCCAACAGGTCGCTATATCCACCAATAGAAGTTTTCCCAAAAA |
| | AGATTCTAGGAACAGTTCTACCACCAGTAATTTGTTCAAAATAGTCACGCAATTCATTTTCGGGTTTAAA |
| | TTCTTTAATATCGACAATTTCATACGCTCCTCTTTTGAAACTAAACTTATTTAGAATATCCAGTGCATTT |
| | CTACAAAAAGGACATGTATACTTGACAAAAATTGTCACTTTGTTATTGGCCAACCTTTGTTGTACAAATT |
| | CCTCGGCCATTTTAATATTTAAGTGATATAAAACTATCTCGACTTATTTAACTCTTTAGTCGAGATATAT |
| | GGACGCAGATAGCTATATGATAGCCAACTACAGAAGGCAAACGCTATAAAAAACATAATTACGACGAGCA |
| | TATTTATAAATATTTTTATTCAGCATTACTTGATATAGTAATATTAGGCACAGTCAAACATTCAACCACT |
| | CTCGATACATTAACTCTCTCATTTTCTTTAACAAATTCTGCAATATCTTCGTAAAAAGATTCTTGAAACT |
| | TTTTAGAATATCTATCGACTCTAGATGAAATAGCGTTCGTCAACATACTATGTTTTGTATACATAAAGGC |
| | GCCCATTTTAACAGTTTCTAGTGACAAAATGCTAGCGATCCTAGGATCCTTTAGAATCACATAGATTGAC |
| | GATTCGTCTCTCTTAGTAACTCTAGTAAAATAATCATACAATCTAGTACGCGAAATAATATTATCCTTGA |
| | CTTGAGGAGATCTAAACAATCTAGTTTTGAGAACATCGATAAGTTCATCGGGAATGACATACATACTATC |
| | TTTAATAGAACTCTTTTCATCCAGTTGAATGGATTCGTCCTTAACCAACTGATTAATGAGATCTTCTATT |
| | TTATCATTTTCCAGATGATATGTATGTCCATTAAAGTTAAATTGTGTAGCGCTTCTTTTTAGTCTAGCAG |
| | CCAATACTTTAACATCACTAATATCGATATACAAAGGAGATGATTTATCTATGGTATTAAGAATTCGTTT |
| | TTCGACATCTGTCAAAACCAATTCCTTTTTGCCTGTATCATCCAGTTTTCCATCCTTTGTAAAGAAATTA |
| | TTTTCTACTAGACTATTAATAAGACTGATAAGGATTCCTCCATAATTGCACAATCCAAACTTTTTCACAA |
| | AACTAGACTTTACAAGATCTACAGGAATGCGTACTTCAGGTTTCTTAGCTTGTGATTTTTTCTTTTGTGG |
| | ACATTTTCTTGTGACCAACTCATCTACCATTTCATTGATTTTAGCAGTGAAATAAGCTTTCAATGCACGG |
| | GCACTGATACTATTGAAAACGAGTTGATCTTCAAATTCCGCCATTTAAGTTCACCAAACAACTTTTAAAT |
| | ACAAATATATCAATAGTAGTAGAATAAGAACTATAAAAAAAATAATAATTAACCAATACCAACCCCAACA |
| | ACCGGTATTATTAGTTGATGTGACTGTTTTCTCATCACTTAGAACAGATTTAACAATTTCTATAAAGTCT |
| | GTCAAATCATCTTCCGGAGACCCCATAAATACACCAAATATAGCGGCGTACAACTTATCCATTTATACAT |
| | TGAATATTGGCTTTTCTTTATCGCTATCTTCATCATATTCATCATCAATATCAACAAGTCCCAGATTACG |
| | AGCCAGATCTTCTTCTACATTTTCAGTCATTGATACACGTTCACTATCTCCAGAGAGTCCGATAACGTTA |
| | GCCACCACTTCTCTATCAATGATTAGTTTCTTGAGTGCGAATGTAATTTTTGTTTCCGTTCCGGATCTAT |
| | AGAAAACTACAGGTGTGATAATTGCCTTGGCCAATTGTCTTTCTCTTTTACTGAGTGATTCTAGTTCACC |
| | TTCTATAGATCTGAGAATGGATGATTCTCCAGTCGAAACATATTCTACCATGGCTCCGTTTAATTTGTTG |
| | ATGAAGATGGATTCATCCTTAAATGTTTTCTCTGTAATAGTTTCCACCGAAAGACTATGCAAAGAATTTG |
| | GAATGCGTTCCTTGTGCTTAATGTTTCCATAGACGGCTTCTAGAAGTTGATACAACATAGGACTAGCCGC |
| | GGTAACTTTTATTTTTAGAAAGTATCCATCGCTTCTATCTTGTTTAGATTTATTTTTATAAAGTTTAGTC |
| | TCTCCTTCCAACATAATAAAAGTGGAAGTCATTTGACTAGATAAACTATCAGTAAGTTTTATAGAGATAG |
| | ACGAACAATTAGCGTATTGAGAAGCATTTAGTGTAACGTATTCGATACATTTTGCATTAGATTTACTAAT |
| | CGATTTTGCATACTCTATAACACCCGCACAAGTCTGTAGAGAATCGCTAGATGCAGTAGGTCTTGGTGAA |
| | GTTTCAACTCTCTTCTTGATTACCTTACTCATGATTAAACCTAAATAATTGTACTTTGTAATATAATGAT |
| | ATATATTTTCACTTTATCTCATTTGAGAATAAAAATGTTTTTGTTTAACCACTGCATGATGTACAGATTT |
| | CGGAATCACAAACCACCGGTGGTTTTATTTTATCCTTGTCCAATGTGAATTGAATGGGAGCGGATGCGGG |
| | TTTCGTACGTAGATAGTACATTCCCGTTTTTAGACCGAGACTCCATCCGTAAAAATGCATACTCGTTAGT |
| | TTGGAATAACTCGGATCTGCTATATGGATATTCATAGATTGACTTTGATCGATGAAGGCTCCCCTGTCTG |
| | CAGCCATTTTTATGATCGTCTTTTGTGGAATTTCCCAAATAGTTTTATAAACTCGCTTAATATCTTCTGG |
| | AAGGTTTGTATTCTGAATGGATCCACCATCTGCCATAATCCTATTCTTGATCTCATCATTCCATAATTTT |
| | CTCTCGGTTAAAACTCTAAGGAGATGCGGATTAACTACTTGAAATTCTCCAGACAATACTCTCCGAGTGT |
| | AAATATTACTGGTATACGGTTCCACCGACTCATTATTTCCCAAAATTTGAGCAGTTGATGCAGTCGGCAT |
| | AGGTGCCACCAATAAACTATTTCTAAGACCGTATGTTCTGATTTTATCTTTTAGAGGTTCCCAATTCCAA |
| | AGATCCGACGGTACAACATTCCAAAGATCATATTGTAGAATACCGTTACTGGCGTACGATCCTACATATG |
| | TATCGTATGGTCCTTCCTTCTCAGCTAGTTCACAACTCGCCTCTAATGCACCGTAATAAATGGTTTCGAA |
| | GATCTTCTTATTTAGATCTTGTGCTTCCAGGCTATCAAATGGATAATTTAAGAGAATAAACGCGTCCGCT |
| | AATCCTTGAACACCAATACCGATAGGTCTATGTCTCTTATTAGAGATTTCAGCTTCTGGAATAGGATAAT |
| | AATTAATATCTATAATTTTATTGAGATTTCTGACAATTACTTTGACCACATCCTTCAGTTTGAGAAAATC |
| | AAATCGCCCATCTATTACAAACATGTTCAAGGCAACAGATGCCAGATTACAAACGGCTACCTCATTAGCA |
| | TCCGCATATTGTATTATCTCAGTGCAAAGATTACTACACTTGATAGTTCCTAAATTTTGTTGATTACTCT |
| | TTTTGTTACACGCATCCTTATAAAGAATGAATGGAGTACCAGTTTCAATCTGAGATTCTATAATCGCTTT |
| | CCAGACGACTCGAGCCTTTATTATAGATTTGTATCTCCTTTCTCTTTCGTATAGTGTATACAATCGTTCG |
| | AACTCGTCTCCCCAAACATTGTCCAATCCAGGACATTCATCCGGACACATCAACGACCACTCTCCGTCAT |
| | CCTTCACTCGTTTCATAAAGAGATCAGGAATCCAAAGAGCTATAAATAGATCTCTGGTTCTATGTTCCTC |
| | GTTTCCTGTATTCTTTTTAAGATCGAGGAACGCCATAATATCAGAATGCCACGGTTCCAAGTATATGGCC |
| | ATAACTCCAGGCCGTTTGTTTCCTCCCTGATCTATGTATCTAGCGGTGTTATTATAAACTCTCAACATTG |
| | GAATAATACCGTTTGATATACCATTGGTACCGGAGATATAGCTTCCACTGGCACGAATATTACTAATTGA |
| | TAGACCTATTCCCCCTGCCATTTTAGAGATTAATGCGCATCGTTTTAACGTGTCATAGATACCCTCTATG |
| | CTATCATCGATCATGTTAAGTAGAAAACAGCTAGACATTTGGTGACGACTAGTTCCCGCATTAAATAAGG |
| | TAGGAGAAGCGTGCGTAAACCATTTTTCAGAAAGTAGATTGTACGTCTCAATAGCTGAGTCTATATCCCA |
| | TTGATGAATTCCTACTGCGACACGCATTAACATGTGCTGAGGTCTTTCAACGATCTTGTTGTTTATTTTC |
| | AACAAGTAGGATTTTTCCAAAGTTTTAAAACCAAAATAGTTGTATGAAAAGTCTCGTTCGTAAATAATAA |
| | CCGAGTTGAGTTTATCCTTATATTTGTTAACTATATCCATGGTGATACTTGAAATAATCGGAGAATGTTT |
| | CCCATTTTTAGGATTAACATAGTTGAATAAATCCTCCATCACTTCACTAAATAGTTTTTTTGTTTCCTTG |
| | TGTAGATTTGATACGGCTATTCTGGCGGCTAGAATGGCATAATCCGGATGTTGTGTAGTACAAGTGGCTG |
| | CTATTTCGGCTGCCAGAGTGTCCAATTCTACCGTTGTTACTCCATTATATATTCCTTGAATAACCTTCAT |
| | AGCTATTTTAATAGGATCTATATGATCCGTGTTTAAGCCATAACATAATTTTCTAATACGAGACGTGATT |
| | TTATCAAACATGACATTTTCCTTGTATCCATTTCGTTTAATGACAAACATTTTTGTTGGTGTAATAAAAA |
| | AATTATTTAACTTTTCATTAATAGGGATTTGACGTACGTAGCGTACAAAATGATTGTTCCTGGTATATAG |
| | ATAAAGAGTCCTATATATTTGAAAATCGTTACGGCTCGATTAAACTTTAATGATTGCATAGTGAATATAT |
| | CATTAGGATTTAACTCCTTGACTATCAGGGCGGCACCAGAAATTACCATCAAAAGCATTAATACAGTTAT |
| | GCCTATCGCAGTTAGAACGGTTATAGCATCCACCATTTATATCTAAAAATTAGATCAAAGAATATGTGAC |
| | AAAGTCCTAGTTGTATATTGAGAATTGACAAAACAATGTTTCTTACATATTTTTTTTTTATTAGTAACCG |
| | ACTTAATAGTAGGAACTGGAAAACTAGACTTGATTATTCTATAAGTATAGATACCCTTCCAAATAATATT |
| | CTCTTTGATAAAAGTTCCAGAAAATGTAGAATTTTTTAAAAAGTTATCTTTTGCTATTACCAAGATTGTG |
| | TTTAGACGCTTATTATTAATATGAGTGATGAAATCCACACCGCCTCTAGATATCGCCTTTATTTCCACAT |
| | TAGATGGTAAATCCAATAGTGAAACTATCTTTTTAGGAATGTATGGACTCGCGTTTAGAGGAGTGAACGT |
| | CTTGGGCGTCGGAAAGGATGATTCGTCAAACGAATAAACAATTTCACAAATGGATGTTAATGTATTAGTA |
| | GGAAATTTTTTGACGCTAGTGGAATTGAAAATTCTAATGGATGATGTTCTACCTATTTCATCCGATAACA |
| | TGTTAATTTCCGACACCAACGGTTTTAATATTTCGATGATATACGGTAGTCTCTCTTTCGGACTTATATA |
| | GCTTATTCCACAATACGAGTCATTATATACTCCAAAAAACAAAATAACTAGTATAAAATCTGTATCGAAT |
| | GGGAAAAACGAAATTATCGACATAGGTATAGAATCCGGAACATTGAACGTATTAATACTTAATTCTTTTT |
| | CTGTGGTAAGTACCGATAGGTTATTGACATTGTATGGTTTTAAATATTCTATAACTTGAGACTTGATAGA |
| | TATTAGTGATGAATTGAAAATTATTTTTATCACCACGTGTGTTTCAGGATCATCGTCGACGCCCGTCAAC |
| | CAACCGAACGGAGTAAAATAAATATCATTAATATATGCTCTAGATATTAGTATTTTTATCAATCCTTTGA |
| | TTATCATCTTCTCGTAGGCGAATGATTCCATGATCAAGAGTGATTTAAGAACATCCTCCGGAGTATTAAT |
| | GGGCTTAGTAAACAGTCCATCGTTGCAATAATAAAAGTTATCCAAGTTAAAGGATATTATGCATTCGTTT |
| | AAAGATATCACCTCATCTGACGGAGACAATTTTTTGGTAGGTTTTAGAGACTTTGAAGCTACTTGTTTAA |
| | CAAAGTTATTCATCGTCGTTTACTATTCTATTTAATTTTGTAGTTAATTTATCACATATCACATTAATTG |
| | ACTTTTTGGTCCATTTTTCCATACGTTTATATTCTTTTAATCCTGCGTTATCCGTTTCCGTTATATCCAG |
| | TGATAGATCGTGCAGGTTAAATAGAATGCTCTTAAATAATGTCATTTTTTTATCCGCTAAAAATTTAAAG |
| | AATGTATAAACTTTTTTCAAAGATTTAAAACTTTTAGGTGGTGTCCTAGTACACAATATCATAAACAAAC |
| | TAATAAACATCCCGCATTCAGATTCCAACAGCTGATTAACTTCCACATTAATACAGCCTATTTTCGCTCC |
| | AAATGTACATTCGAAAAATCTGAATAAAACATCAATGTCGCAATTTGTATTATCCAATACAGAATGTTTG |
| | TGATTCGTGTTAAAACCATCGGAGAAGGAATAGAAATAAAAATTATTATAGTGGTGGAATTCAGTTGGAA |
| | TATTGCCTCCGGAGTCATAAAAGGATACTAAACATTGTTTTTTATCATAAATTACACATTTCCAATGAGA |
| | CAAATAACAAAATCCAAACATTACAAATCTAGAGGTAGAACTTTTAATTTTGTCTTTAAGTATATACGAT |
| | AAGATATGTTTATTCATAAACGCGTCAAATTTTTCATGAATCGCTAAGGAGTTTAAGAATCTCATGTCAA |
| | ATTGTCCTATATAATCCACTTCGGATCCATAAGCAAACTGAGAGACTAAGTTCTTAATACTTCGATTGCT |
| | CATCCAGGCTCCTCTCTCAGGCTCTATTTTCATCTTGACGACCTTTGGATTTTCACCAGTATGTATTCCT |
| | TTACGTGATAAATCATCGATTTTCAAATCCATTTGTGAGAAGTCTATCGCCTTAGATACTTTTTCCCGTA |
| | GTCGAGGTTTAAAGAAATACGCTAACGGTATACTAGTAGGTAACTCAAAAACATCATATATAGAATGGTA |
| | ACGCGTCTTTAACTCGTCGGTTAACTCTTTCTTTTGATCGAGTTCGTCGCTACTATTGGGTCTGCTCAGG |
| | TGCCCCGACTCTACTAGTTCCAACATCATACCGATAGGAATACAAGACACTTTGCCGGCGGTTGTAGATT |
| | TATCATATTTTTCCACTACATATCCGTTACAATTTGTTAAAAATTTAGATACATCTATATTGCTACATAA |
| | TCCAGCTAGTGAATATATATGACATAATAAATTGGTAAATCCTAGTTCTGGTATTTTACTAATTACTAAA |
| | TCTGTATATCTTTCCATTTATCATGGAAAAGAATTTACCAGATATCTTCTTTTTTCCAAACTGCGTTAAT |
| | GTATTCTCTTACAAATATTCACAAGATGAATTCAGTAATATGAGTAAAACGGAACGTGATAGTTTCTCAT |
| | TGGCCGTGTTTCCAGTTATAAAACATAGATGGCATAACGCACACGTTGTAAAACATAAAGGAATATACAA |
| | AGTTAGTACAGAAGCACGTGGAAAAAAAGTATCTCCTCCATCACTAGGAAAACCCGCACACATAAACCTA |
| | ACCGCGAAGCAATATATATACAGTGAACACACAATAAGCTTTGAATGTTATAGTTTTCTAAAATGTATAA |
| | CAAATACAGAAATCAATTCGTTCGATGAGTATATATTAAGAGGACTATTAGAAGCTGGTAATAGTTTACA |
| | GATATTTTCCAATTCCGTAGGTAAACGAACAGATACTATAGGTGTACTAGGGAATAAGTATCCATTTAGC |
| | AAAATTCCATTGGCCTCATTAACTCCTAAAGCACAACGAGAGATATTTTCAGCGTGGATTTCTCATAGAC |
| | CTGTAGTTTTAACTGGAGGAACTGGAGTGGGTAAGACGTCACAGGTACCCAAGTTATTGCTTTGGTTTAA |
| | TTATTTATTTGGTGGATTCTCTACTCTAGATAAAATCACTAACTTTCACGAAAGACCAGTCATTCTATCT |
| | CTTCCTAGGATAGCTTTAGTTAGATTGCATAGCAATACCATTTTAAAATCATTGGGATTTAAGGTACTAG |
| | ATGGATCTCCTATTTCTTTACGGTACGGATCTATACCGGAAGAATTAATAAACAAACAACCAAAAAAATA |
| | TGGAATTGTATTTTCTACCCATAAGTTATCTCTAACAAAACTATTTAGTTATGGCACTCTTATTATAGAC |
| | GAAGTTCATGAGCATGATCAAATAGGAGATATTATTATAGCAGTAGCGAGAAAGCATCATACGAAAATAG |
| | ATTCTATGTTTTTAATGACTGCCACGTTAGAGGATGACCGAGAACGGCTAAAAGTATTTTTACCTAATCC |
| | CGCATTTATACATATTCCTGGAGATACACTGTTTAAAATTAGCGAGGTATTTATTCATAATAAGATAAAT |
| | CCATCTTCCAGAATGGCATACATAGAAGAAGAAAAGAGAAATTTAGTTACTGCTATACAGATGTATACTC |
| | CTCCTGATGGATCATCCGGTATAGTCTTTGTGGCATCCGTTGCACAGTGTCACGAATATAAATCATATTT |
| | AGAAAAAAGATTACCGTATGATATGTATATTATTCATGGTAAGGTCTTAGATATAGACGAAATATTAGAA |
| | AAAGTGTATTCATCACCTAATGTATCGATAATTATTTCTACTCCTTATTTGGAATCCAGCGTTACTATAC |
| | GCAATGTTACACACATTTATGATATGGGTAAAGTTTTTGTCCCCGCTCCTTTTGGAGGATCGCAAGAATT |
| | TATTTCTAAATCTATGAGAGATCAACGAAAAGGAAGAGTAGGAAGAGTTAATCCTGGTACATACGTCTAT |
| | TTCTATGATCTGTCTTATATGAAGTCTATACAGCGAATAGATTCAGAATTTCTACATAATTATATATTGT |
| | ACGCTAATAAGTTTAATCTAACACTCCCCGAAGATTTGTTTATAATCCCTACAAATTTGGATATTCTATG |
| | GCGTACAAAGGAATATATAGACTCGTTCGATATTAGTACAGAAACATGGAATAAATTATTATCCAATTAT |
| | TATATGAAGATGATAGAGTATGCTAAACTTTATGTACTAAGTCCTATTCTCGCTGAGGAGTTGGATAACT |
| | TTGAGAGGACGGGAGAATTAACTAGTATTGTACGAGAAGCCATTTTATCTCTAAATTTACGAATTAAGAT |
| | TTTAAATTTTAAACATAAAGATGATGATACGTATATACACTTTTGTAAAATATTATTCGGTGTCTATAAC |
| | GGAACAAACGCTACTATATATTATCATAGACCTCTAACGGGATATATGAATATGATTTCAGATACTATAT |
| | TTGTTCCTGTAGATAATAACTAAAAATCAAACTCTAATGACCACATCTTTTTTTAGAGATGAAAAATTTT |
| | CTACATCTCCTTTTGTAGACACGACTAAACATTTTGCAAAAAAAAGTTTATTAGTGTTTAGATAATCGTA |
| | TACTTCATCAGTGTAGATAGTAAATGTGAACAAATAAAAGGTATTCTTACTCAATAGATTGGTAAATTCC |
| | ATAGAATATATTAATCCTTTCTTCTTGAGATCCCACATCATTTCAACCAGAGACGTTTTATCCAATGATT |
| | TACCTCGTACTATACCACATACAAAACTAGATTTTGCAGTGACGTCGTACCTGGTATTCCTACCAAACAA |
| | AATTTTACTTTTAGTTCTTTTAGAAAATTCTAAGGTAGAATCTCTATTTGCCAATATGTCATCTATGGAA |
| | TTACCACTAGCAAAAAATGATAGAAATATATATTGATACATCGCAGCTGGTTTTGATCTACTATACTTTA |
| | AAAACGAATCAGATTCCATAATTGCCTGTATATCATCAGCTGAAAAACTATGTTTTACACGTATTCCTTC |
| | GGCATTTCTTTTTAATGATATATCTTGTTTAGACAATGATAAAGTTATCATGTCCATGAGAGACGCGTCT |
| | CCGTATCGTATAAATATTTCATTAGATGTTAGACGCTTCATTAGGGGTATACTTCTATAAGGTTTCTTAA |
| | TCAGTCCATCATTGGTTGCGTCAAGAACTACTATCGGATGTTGTTGGGTATCTCTAGTGTTACACATGGC |
| | CTTACTAAAGTTTGGGTAAATAACTATGATATCTCTATTAATTATAGATGCATATATTTCATTCGTCAAG |
| | GATATTAGTATCGACTTGCTATCGTCATTAATACGTGTAATGTAATCATATAAATCATGCGATAGCCAAG |
| | GAAAATTCAAATAGATGTTCATCATATAATCGTCGCTATAATTCATATTAATACGTTGACATTGACTAAT |
| | TTGTAATATAGCCTCGCCACGAAGAAAGCTCTCGTATTCAGTTTCATCGATAAAGGATACCGTTAAATAT |
| | AACTGGTTGCCGATAGTCTCATAGTCTATTAAGTGGTAAGTTTCGTACAAATACAGAATCCCTAAAATAT |
| | TATCTAATGTTGGATTAATCTTTACCATAACTGTATAAAATGGAGACGGAGTCATAACTATTTTACCGTT |
| | TGTACTTACTGGAATAGACGAAGGAATAATCTCCGGACATGCTGGTAAAGACCCAAATGTCTGTTTGAAG |
| | AAATCCAATGTTCCAGGTCCTAATCTCTTAACAAAAATTACGATATTCGATCCCGATATCCTTTGCATTC |
| | TATTTACCAGCATATCACGAACTATATTAAGATTATCTATCATGTCTATTCTCCCACCGTTATATAAATC |
| | GCCTCCGCTAAGAAACGTTAGTATATCCATACAATGGAATACTTCATTTCTAAAATAGTATTCGTTTTCT |
| | AATTCTTTAATGTGAAATCGTATACTAGAAAGGGAAAAATTATCTTTGAGTTTTCCGTTAGAAAAGAACC |
| | ACGAAACTAATGTTCTGATTGCGTCCGATTCCGTTGCTGAATTAATGGATTTACACCAAAAACTCATATA |
| | ACTTCTAGATGTAGAAGCATTCGCTAAAAAATTAGTAGAATCAAAGGATATAAGTAGATGTTCCAACAAG |
| | TGAGCAATTCCCAAGATTTCATCTATATCATTCTCGAATCCGAAATTAGAAATTCCCAAGTAGATATCCT |
| | TTTTCATCCGATCGTTGATGAAAATACGAACTTTATTCGGTAAGACAATCATTTACTAAGGAGTAAAATA |
| | GGAAGTAATGTTCGTATGTCGTTATCATCGTATAAATTAAAGGTGTGTTTTTTACCATTAAGTGACATTA |
| | TAATTTTACCAATATTGGAATTATAATATAGGTGTATTTGCGCACTCGCGACGGTTGATGCATCGGTAAA |
| | TATAGCTGTATCTAATGTTCTAGTCGGTATTTCATCATTTCGCTGTCTAATAATAGCGTTTTCTCTATCT |
| | GTTTCCATTACAGCTGCCTGAAGTTTATTGGTCGGATAATATGTAAAATAATAAGAAATACATACGAATA |
| | ACAAAAATAAAATAAGATATAATAAAGATGCCATTTAGAGATCTAATTTTGTTCAACTTGTCCAAATTCC |
| | TACTTACAGAAGATGAGGAATCGTTGGAGATAGTGTCTTCCTTATGTAGAGGATTTGAAATATCTTATGA |
| | TGACTTGATAACTTACTTTCCAGATAGGAAATACCATAAATATATTTCTAAAGTATTTGAACATGTAGAT |
| | TTATCGGAGGAATTAAGTATGGAATTCCATGATACAACTTTGCGAGATTTAGTCTATCTTAGATTGTACA |
| | AGTATTCCAAGTGTATACGGCCGTGTTATAAATTAGGAGATAATCTAAAAGGCATAGTTGTTATAAAGGA |
| | CAGGAATATTTATATTAGGGAAGCAAATGATGACTTGATAGAATATCTCCTCAAGGAATACACTCCTCAG |
| | ATTTATACATATTCTAATGAGCGCGTCCCCATAACTGGTTCAAAATTAATTCTTTGTGGATTTTCTCAAG |
| | TTACATTTATGGCGTATACAACGTCGCATATAACAACAAATAAAAAGGTAGATGTTCTCGTTTCCAAAAA |
| | ATGTATAGATGAACTAGTCGATCCAATAAATTATCAAATACTTCAAAATTTATTTGATAAAGGAAGCGGA |
| | ACAATAAACAAAATACTCAGGAAGATATTTTATTCGGTAACAGGTGGCCAAACTCCATAGGTAGCTTTTT |
| | CTATTTCGGATTTTAGAATTTCCAAATTCACCAGCGATTTATCTGTTTTGGTGAAATCCAAGGATTTATT |
| | AATGTCCACAAATGCCATTTGTTTTGTCTGTGGATTGTATTTGAAAATGGAAACGATGTAGTTAGATAGA |
| | TGCGCTGCGAAGTTTCCTATTAGGGTTCCGCGCTTCACGTCACCCAGCATACTTGAATCACCATCCTTTA |
| | AAAAAAATGATAAGATATCAACATGGAGTATATCATACTCGGATTTTAATTCTTCTACTGCATCACTGAC |
| | ATTTTCACAAATACTACAATACGGTTTACCGAAAATAATCAGTACGTTCTTCATTTATGGGTATCAAAAA |
| | CTTAAAATCGTTACTGCTGGAAAATAAATCACTGACGATATTAGATGATAATTTATACAAAGTATACAAT |
| | GGAATATTTGTGGATACAATGAGTATTTATATAGCCGTCGCCAATTGTGTCAGAAACTTAGAAGAGTTAA |
| | CTACGGTATTCATAAAATACGTAAACGGATGGGTAAAAAAGGGAGGGCATGTAACCCTTTTTATCGATAG |
| | AGGAAGTATAAAAATTAAACAAGACGTTAGAGACAAGAGACGTAAATATTCTAAATTAACCAAGGACAGA |
| | AAAATGCTAGAATTAGAAAAGTGTACATCCGAAATACAAAATGTTACCGGATTTATGGAAGAAGAAATAA |
| | AGGCAGAAATGCAATTAAAAATCGATAAACTCACATTTCAAATATATTTATCTGATTCTGATAACATAAA |
| | AATATCATTGAATGAGATACTAACACATTTCAACAATAATGAGAATGTTACATTATTTTATTGTGATGAA |
| | CGAGACGCAGAATTCGTTATGTGTCTCGAGGCTAAAACACATTTCTCTACCACAGGAGAATGGCCGTTGA |
| | TAATAAGTACCGATCAGGATACTATGCTATTTGCATCTGCTGATAATCATCCTAAGATGATAAAAAACTT |
| | AACTCAACTGTTTAAATATGTTCCATCTGCAGAGGATAACTATTTAGCAAAATTAACGGCGTTAGTGAAT |
| | GGATGTGATTTCTTTCCTGGACTCTATGGGGCATCTATAACACCCAACAACTTAAACAAAATACAATTGT |
| | TTAGTGATTTTACAATCGATAATATAGTCACTAGTTTGGCAATTAAAAATTATTATAGAAAGACTAACTC |
| | TACCGTAGACGTGCGTAATATTGTTACGTTTATAAACGATTACGCTAATTTAGACGATGTCTACTCGTAT |
| | ATTCCTCCTTGTCAATGCACTGTTCAAGAATTTATATTTTCCGCATTAGATGAAAAATGGAATGAATTTA |
| | AATCATCTTATTTAGAAAGCGTGCCGTTACCCTGCCAATTAATGTATGCGTTAGAACCACGCAAGGAGAT |
| | TGATGTTTCAGAAGTTAAAACTTTATCATCTTATATAGATTTCGAAAATACTAAATCAGATATCGATGTT |
| | ATAAAATCTATATCCTCGATCTTCGGATATTCTAACGAAAACTGTAACACGATAGTATTCGGCATCTATA |
| | AGGATAATTTACTACTGAGTATAAATAGTTCATTTTACTTTAACGATAGTCTGTTAATAACCAATACTAA |
| | AAGTGATAATATAATAAATATAGGTTACTAGATTAAAAATGGTGTTCCAACTCGTGTGCTCTACGTGCGG |
| | CAAAGATATTTCTCACGAACGATATAAATTGATTATACGAAAAAAATCATTAAAGGATGTACTCGTCAGT |
| | GTAAAGAACGAATGTTGTAGGTTAAAATTATCTACACAAATAGAACCTCAACGTAACTTAACAGTGCAAC |
| | CTCTATTGGATATAAACTAATATGGATCCGGTTAATTTTATCAAGACATATGCGCCTAGAGGTTCTATTA |
| | TTTTTATTAATTATACCATGTCATTAACAAGTCATTTGAATCCATCGATAGAAAAACATGTGGGTATTTA |
| | TTATGGTACGTTATTATCGGAACACTTGGTAGTTGAATCAACATATAGAAAAGGAGTTCGAATAGTCCCA |
| | TTGGATAGTTTTTTTGAAGGATATCTTAGTGCAAAAGTATACATGTTAGAGAATATTCAAGTTATGAAAA |
| | TAGCAGCTGATACGTCATTAACTTTATTGGGTATTCCGTATGGATTTGGTCATGATAGAATGTATTGTTT |
| | TAAATTGGTAGCTGACTGTTATAAAAATGCCGGTATTGATACATCGTCTAAACGAATATTAGGTAAAGAT |
| | ATTTTTCTGAGCCAAAACTTCACAGACGATAATAGATGGATAAAGATATATGATTCTAATAATTTAACAT |
| | TTTGGCAAATTGATTACCTTAAAGGGTGAGTTAATATGCATAACTACTCCTCCGTTGTTTTTTCCCTCGT |
| | TCTTTTTCTTAACGTTGTTTGCCATCACTCTCATAATGTAAAGATATTCTAAAATGGTAAACTTTTGCAT |
| | ATCGGACGCAGAAATTGGTATAAATGTTGTAATTGTATTATTTCCCGTCAATGGACTAGTCACAGCTCCA |
| | TCAGTTTTATATCCTTTAGAGTATTTCTCACTCGTGTCTAACATTCTAGAGCATTCCATGATCTGTTTAT |
| | CGTTGATATTGGCCGGAAAGATAGATTTTTTATTTTTTATTATATTACTATTGGCAATTGTAGATATAAC |
| | TTCTGGTAAATATTTTTCTACCTTTTCAATCTCTTCTATTTTCAAGCCGGCTATATATTCTGCTATATTG |
| | TTGCTAGTATCAATACCTTTTCTGGCTAAGAAGTCATATGTGGTATTCACTATATCAGTTTTAACTGGTA |
| | GTTCCATTAGCCTTTCCACTTCTGCAGAATAATCAGAAATTGGTTCTTTACCAGAAAATCCAGCTACTAT |
| | AATAGGCTCACCGATGATCATTGGCAAAATCCTATATTGTACCAGATTAATGAGAGCATATTTCATTTCC |
| | AATAATTCTGCTAGTTCTTGAGACATTGATTTATTTGATGAATCTAGTTGGTTCTCTAGATACTCTACCA |
| | TTTCTGCCGCATACAATAACTTGTTAGATAAAATCAGGGTTATCAAAGTGTTTAGCGTGGCTAGAATAGT |
| | GGGCTTGCATGTATTAAAGAATGCGGTAGTATGAGTAAACCGTTTTAACGAATTATATAGTCTCCAGAAA |
| | TCTGTGGCGTTACATACATGAGCCGAATGACATCGAAGATTGTCCAATATTTTTAATAGCTGCTCTTTGT |
| | CCATTATTTCTATATTTGACTCGCAACAATTGTAGATACCATTAATCACCGATTCCTTTTTCGATGCCGG |
| | ACAATAGCACAATTGTTTAGCTTTGGACTCTATGTATTCAGAATTAATAGATATATCTCTTAATACAGAT |
| | TGCACTATACATTTTGAAACTATGTCAAAAATTGTAGAACGACGCTGTTCTGCAGCCATTTAACTTTAAA |
| | TAATTTACAAAAATTTAAAATGAGCATCCGTATAAAAATCGATAAACTGCGCCAAATTGTGGCATATTTT |
| | TCAGAGTTCAGTGAAGAAGTATCTATAAATGTAGACTCGACGGATGAGTTAATGTATATTTTTGCCGCCT |
| | TGGGCGGATCTGTAAACATTTGGGCCATTATACCTCTCAGTGCATCAGTGTTCTACCGCGGAGCCGAAAA |
| | TATTGTGTTTAATCTTCCTGTGTCCAAGGTAAAATCGTGTTTGTGTAGTTTTCACAATGATGCCATCATA |
| | GATATAGAACCTGATCTGGAAAATAATCTAGTAAAACTTTCTAGTTATCATGTAGTAAGTGTCGATTGTA |
| | ATAAGGAACTGATGCCTATTAGGACAGATACTACTATTTGTCTAAGTATAGATCAAAAGAAATCTTACGT |
| | GTTTAATTTTCACAAGTATGAAGAAAAATGTTGTGGTAGAACCGTCATTCATTTAGAATGGTTGTTGGGC |
| | TTTATCAAGTGTATTAGTCAGCATCAGCATTTGGCTATTATGTTTAAAGATGACAATATTATTATGAAGA |
| | CTCCTGGTAATACTGATGCATTTTCCAGGGAATATTCTATGACTGAATGTTCTCAAGAACTACAAAAGTT |
| | TTCTTTCAAAATAGCTATCTCGTCTCTCAACAAACTACGAGGATTCAAAAAGAGAGTCAATGTTTTTGAA |
| | ACTAGAATCGTAATGGATAATGACGATAACATTTTAGGAATGTTGTTTTCGGATAGAGTTCAATCCTTTA |
| | AGATCAACATCTTTATGGCGTTTTTAGATTAATACTTTCAATGAGATAAATATGGGTGGCGGAGTAAGTG |
| | TTGAGCTCCCTAAACGGGATCCGCCTCCGGGAGTACCCACTGATGAGATGTTATTAAACGTGGATAAAAT |
| | GCATGACGTGATAGCTCCCGCTAAGCTTTTAGAATATGTGCATATAGGACCACTAGCAAAAGATAAAGAG |
| | GATAAAGTAAAGAAAAGATATCCAGAGTTTAGATTAGTCAACACAGGACCCGGTGGTCTTTCGGCATTGT |
| | TAAGACAATCGTATAATGGAACCGCACCCAATTGCTGTCGCACTTTTAATCGTACTCATTATTGGAAGAA |
| | GGATGGAAAGATATCAGATAAGTATGAAGAGGGTGCAGTATTAGAATCGTGTTGGCCAGACGTTCACGAC |
| | ACCGGAAAATGCGATGTTGATTTATTCGACTGGTGTCAGGGGGATACGTTCGATAGAAACATATGCCATC |
| | AGTGGATCGGTTCAGCCTTTAATAGGAGTAATAGAACTGTAGAGGGTCAACAATCGTTAATAAATCTGTA |
| | TAATAAGATGCAAACATTATGTAGTAAAGATGCTAGTGTACCAATATGTGAATCATTTTTGCATCATTTA |
| | CGCGCACACAATACAGAAGATAGCAAAGAGATGATCGATTATATTCTAAGACAACAGTCTGCGGACTTTA |
| | AACAGAAATATATGAGATGTAGTTATCCCACTAGAGATAAGTTAGAAGAGTCATTAAAATATGCGGAACC |
| | TCGAGAATGTTGGGATCCAGAGTGTTCGAATGCCAATGTTAATTTCTTGCTAACACGTAATTATAATAAT |
| | TTAGGACTTTGCAATATTGTACGATGTAATACTAGCGTGAACAACTTACAGATGGATAAAACTTCCTCAT |
| | TAAGATTGTCATGTGGATTAAGCAATAGTGATAGATTTTCTACTGTTCCCGTCAATAGAGCAAAAGTAGT |
| | TCAACATAATATTAAACACTCGTTCGACCTAAAATTGCATTTGATCAGTTTATTATCTCTCTTGGTAATA |
| | TGGATACTAATTGTAGCTATTTAAATGGGTGCCGCGGCAAGCATACAGACGACGGTGAATACACTCAGCG |
| | AACGTATCTCGTCTAAATTAGAACAAGAAGCGAATGCTAGTGCTCAAACAAAATGTGATATAGAAATCGG |
| | AAATTTTTATATCCGACAAAACCATGGATGTAACCTCACTGTTAAAAATATGTGCTCTGCGGACGCGGAT |
| | GCTCAGTTGGATGCTGTGTTATCAGCCGCTACAGAAACATATAGTGGATTAACACCGGAACAAAAAGCAT |
| | ACGTGCCAGCTATGTTTACTGCTGCGTTAAACATTCAGACGAGTGTAAACACTGTTGTTAGAGATTTTGA |
| | AAATTATGTGAAACAGACTTGTAATTCTAGCGCGGTCGTCGATAACAAATTAAAGATACAAAACGTAATC |
| | ATAGATGAATGTTACGGAGCCCCAGGATCTCCAACAAATTTGGAATTTATTAATACAGGATCTAGCAAAG |
| | GAAATTGTGCCATTAAGGCGTTGATGCAATTGACGACTAAGGCCACTACTCAAATAGCACCTAAACAAGT |
| | TGCTGGTACAGGAGTTCAGTTTTATATGATTGTTATCGGTGTTATAATATTGGCAGCGTTGTTTATGTAC |
| | TATGCCAAGCGTATGTTGTTCACATCCACCAATGATAAAATCAAACTTATTTTAGCCAATAAGGAAAACG |
| | TCCATTGGACTACTTACATGGACACATTCTTTAGAACTTCTCCGATGGTTATTGCTACCACGGATATGCA |
| | AAACTGAAAATATATTGATAATATTTTAATAGATTAACATGGAAGTTATCACTGATCGTCTAGACGATAT |
| | AGTGAAACAAAATATAGCGGATGAAAAATTTGTAGATTTTGTTATACACGGTCTAGAGCATCAATGTCCT |
| | GCTATACTTCGACCATTAATTAGGTTGTTTATTGATATACTATTATTTGTTATAGTAATTTATATTTTTA |
| | CGGTACGTCTAGTAAGTAGAAATTATCAAATGTTGTTGGCGTTGGTGGCGCTAGTCATCACATTAACTAT |
| | TTTTTATTACTTTATACTATAATAGTACTAGACTGACTTCTAACAAACATCTCACCTGCCATAAATAAAT |
| | GCTTGATATTAAAGTCTTCTATTTCTAACACTATTCCATCTGTGGAAAATAATACTCTGACATTATCGCT |
| | AATTGACACATCGGTGAGTGATATGCCTATAAAGTAATAATCTTCTTTGGGCACATATACCAGTGTACCA |
| | GGTTCTAACAACCTATTTACTGGTGCTCCTATAGCATACTTTTTCTTTACCTTGAGAATATCCATCGTTT |
| | GCTTGGTCAATAGCGATATGTGATTTTTTATCAACCACTCGAAAAAGTAATTGGAGTGTTCATATCCTCT |
| | ACGGGCTATTGTCTCATGGCCGTGTATGAAATTTAAGTAACACGACTGTGGTAGATTTGTTCTATAGAGC |
| | CGGTTGCCGCAAATAGATAGAACTACCAATATGTCTGTACAAATGTTAAACATTAATTGATTAACAGAAA |
| | AAACAATGTTCGTTCTGGGAATAGAAACCAGATCAAAACAAAATTCGTTAGAATATATGCCACGTTTATA |
| | CATTGAATATAAAATAACTACAGTTTGAAAAATAACAGTATCATTTAAACATTTAACTTGCGGGGTTAAT |
| | CTCACAACTTTACTGTTTTTGAACTGTTCAAAATATAGCATAGATCCGTGAGAAATACGTTTAGCCGCCT |
| | TTAATAGAGGAAATCCCACCGCCTTTCTGGATCTCACCAACGACGATAGTTCTGACCAGCAACTCATTTC |
| | TTCATCATCCACCTGTTTTAACATATAATAGGCAGGAGATAGATATCCGTCATTGCAATATTCCTTCTCG |
| | TAGGCACACAATCTAATATTGATAAAATCTCCATTCTCTTCTCTGCATTTATTATCTTGTTTCGGTGGCT |
| | GATTAGGCTGTAGTCTTGGTTTAGGCTTTGGTATATCGTTGTTGAATCTATTTTGGTCATTAAATCTTTC |
| | ATTTCTTCCTGGTATATTTTTATCACCTCGTTTGGTTGGATTTTTGTCTATATTATCGTTTGTAACATCG |
| | GTACGGGTATTCATTTATCACAAAAAAAACTTCTCTAAATGAGTCTACTGCTAGAAAACCTCATCGAAGA |
| | AGATACCATATTTTTTGCAGGAAGTATATCTGAGTATGATGATTTACAAATGGTTATTGCCGGCGCAAAA |
| | TCCAAATTTCCAAGATCTATGCTTTCTATTTTTAATATAGTACCTAGAACGATGTCAAAATATGAGTTGG |
| | AGTTGATTCATAACGAAAATATCACAGGAGCAATGTTTACCACAATGTATAATATAAGAAACAATTTGGG |
| | TCTAGGAGATGATAAACTAACTATTGAAGCCATTGAAAACTATTTCTTGGATCCTAACAATGAAGTTATG |
| | CCTCTTATTATTAATAATACGGATATGACTGCCGTCATTCCTAAAAAAAGTGGTAGGAGAAAGAATAAGA |
| | ACATGGTTATCTTCCGTCAAGGATCATCACCTATCTTGTGTATTTTCGAAACTCGTAAAAAGATTAATAT |
| | TTATAAAGAAAATATGGAATCCGCGTCGACTGAGTATACACCTATCGGAGACAACAAGGCTTTGATATCT |
| | AAATATGCGGGAATTAATATCCTAAATGTGTATTCTCCTTCCACATCCATAAGATTGAATGCCATTTACG |
| | GATTCACCAATAAAAATAAACTAGAGAAACTTAGTACTAATAAGGAACTAGAATCGTATAGTTCTAGCCC |
| | TCTTCAAGAACCCATTAGGTTAAATGATTTTCTGGGACTATTGGAATGTGTTAAAAAGAATATTCCTCTA |
| | ACAGATATTCCGACAAAGGATTGATTACTATAAATGGAGAATGTTCCTAATGTATACTTTAATCCTGTGT |
| | TTATAGAGCCCACGTTTAAACATTCTTTATTAAGTGTTTATAAACACAGATTAATAGTTTTATTTGAAGT |
| | ATTCGTTGTATTCATTCTAATATATGTATTTTTTAGATCTGAATTAAATATGTTCTTCATGCCTAAACGA |
| | AAAATACCCGATCCTATTGATAGATTACGACGTGCTAATCTAGCGTGTGAAGACGATAAATTAATGATCT |
| | ATGGATTACCATGGATGACAACTCAAACATCTGCGTTATCAATAAATAGTAAACCGATAGTGTATAAAGA |
| | TTGTGCAAAGCTTTTGCGATCAATAAATGGATCACAACCAGTATCTCTTAACGATGTTCTTCGCAGATGA |
| | TGATTCATTTTTTAAGTATTTGGCTAGTCAAGATGATGAATCTTCATTATCTGATATATTGCAAATCACT |
| | CAATATCTAGACTTTCTGTTATTATTATTGATCCAATCAAAAAATAAATTAGAAGCCGTGGGTCATTGTT |
| | ATGAATCTCTTTCAGAGGAATACAGACAATTGACAAAATTCACAGACTTTCAAGATTTTAAAAAACTGTT |
| | TAACAAGGTCCCTATTGTTACAGATGGAAGGGTCAAACTTAATAAAGGATATTTGTTCGACTTTGTGATT |
| | AGTTTGATGCGATTCAAAAAAGAATCCTCTCTAGCTACCACCGCAATAGATCCTGTTAGATACATAGATC |
| | CTCGTCGCAATATCGCATTTTCTAACGTGATGGATATATTAAAGTCGAATAAAGTGAACAATAATTAATT |
| | CTTTATTGTCATCATGAACGGCGGACATATTCAGTTGATAATCGGCCCCATGTTTTCAGGTAAAAGTACA |
| | GAATTAATTAGACGAGTTAGACGTTATCAAATAGCTCAATATAAATGCGTGACTATAAAATATTCTAACG |
| | ATAATAGATACGGAACGGGACTATGGACGCATGATAAGAATAATTTTGAAGCATTGGAAGCAACTAAACT |
| | ATGTGATGTCTTGGAATCAATTACAGATTTCTCCGTGATAGGTATCGATGAAGGACAGTTCTTTCCAGAC |
| | ATTGTTGAATTCTGTGAGCGTATGGCAAACGAAGGAAAAATAGTTATAGTAGCCGCACTCGATGGGACAT |
| | TTCAACGTAAACCGTTTAATAATATTTTGAATCTTATTCCATTATCTGAAATGGTGGTAAAACTAACTGC |
| | TGTGTGTATGAAATGCTTTAAGGAGGCTTCCTTTTCTAAACGATTGGGTGAGGAAACCGAGATAGAAATA |
| | ATAGGAGGTAATGATATGTATCAATCGGTGTGTAGAAAGTGTTACATCGACTCATAATATTATATTTTTT |
| | ATCTAAAAAACTAAAAATAAACATTGATTAAATTTTAATATAATACTTAAAAATGGATGTTGTGTCGTTA |
| | GATAAACCGTTTATGTATTTTGAGGAAATTGATAATGAGTTAGATTACGAACCAGAAAGTGCAAATGAGG |
| | TCGCAAAAAAACTGCCGTATCAAGGACAGTTAAAACTATTACTAGGAGAATTATTTTTTCTTAGTAAGTT |
| | ACAGCGACACGGTATATTAGATGGTGCCACCGTAGTGTATATAGGATCTGCTCCCGGTACACATATACGT |
| | TATTTGAGAGATCATTTCTATAATTTAGGAGTGATCATCAAATGGATGCTAATTGACGGCCGCCATCATG |
| | ATCCTATTTTAAATGGATTGCGTGATGTGACTCTAGTGACTCGGTTCGTTGATGAGGAATATCTACGATC |
| | CATCAAAAAACAACTGCATCCTTCTAAGATTATTTTAATTTCTGATGTGAGATCCAAACGAGGAGGAAAT |
| | GAACCTAGTACGGCGGATTTACTAAGTAATTACGCTCTACAAAATGTCATGATTAGTATTTTAAACCCCG |
| | TGGCGTCTAGTCTTAAATGGAGATGCCCGTTTCCAGATCAATGGATCAAGGACTTTTATATCCCACACGG |
| | TAATAAAATGTTACAACCTTTTGCTCCTTCATATTCAGCTGAAATGAGATTATTAAGTATTTATACCGGT |
| | GAGAACATGAGACTGACTCGAGTTACCAAATCAGACGCTGTAAATTATGAAAAAAAGATGTACTACCTTA |
| | ATAAGATCGTCCGTAACAAAGTAGTTGTTAACTTTGATTATCCTAATCAGGAATATGACTATTTTCACAT |
| | GTACTTTATGCTGAGGACCGTGTACTGCAATAAAACATTTCCTACTACTAAAGCAAAGGTACTATTTCTA |
| | CAACAATCTATATTTCGTTTCTTAAATATTCCAACAACATCAACTGAAAAAGTTAGTCATGAACCAATAC |
| | AACGTAAAATATCTAGCAAAAATTCTATGTCTAAAAACAGAAATAGCAAGAGATCCGTACGCAGTAATAA |
| | ATAGAAACGTACTACTGAGATATACTACCGATATAGAGTATAATGATTTAGTTACTTTAATAACCGTTAG |
| | ACATAAAATTGATTCTATGAAAACTGTGTTTCAGGTATTTAACGAATCATCCATAAATTATACTCCGGTT |
| | GATGATGATTATGGAGAACCAATCATTATAACATCGTATCTTCAAAAAGGTCATAACAAGTTTCCTGTAA |
| | ATTTTCTATACATAGATGTGGTAATATCTGACTTATTTCCTAGCTTTGTTAGACTAGATACTACAGAAAC |
| | TAATATAGTTAATAGTGTACTACAAACAGGCGATGGTAAAAAGACTCTTCGTCTTCCCAAAATGTTAGAG |
| | ACGGAAATAGTTGTCAAGATTCTCTATCGCCCTAATATACCATTAAAAATTGTTAGATTTTTCCGCAATA |
| | ACATGGTAACTGGAGTAGAGATAGCCGATAGATCTGTTATTTCAGTCGCTGATTAATCAATTAGTAGAGA |
| | TGAGATAAGAACATTATAATAATCAATAATATATCTTATATCTCGTTTAGAAAAATGCTAATATTAAAAT |
| | AGCTAACGCTAGTAATCCAATCGGAAGCCATTTGATATCTATAATAGGGTATCTAATTTCCTGATTCAGA |
| | TAGCGGACAGCTATATTCTCGGTAGCTACTCGTTTGGAATCACAAACATTATTTACATCTAATTTACTAT |
| | CTGTAATGGAAACGTTTCCCAATGAAATGGTACAATCCGATACATTGCATTTTGTTATATTTTTTTTTAA |
| | AGAGGCTGGTAACAACGCATCGCTTCGTTTACATGGCTCGTACCAACAATAATAGGGTAATCTTGTATCT |
| | ATTCCTATCCGTACTATGCTTTTATCAGGATAAATACATTTACATCGTATATCGTCTTTGTTAGCATCAC |
| | AGAATGCATAAATTTGTTCGTCCGTCATGATAAAAATTTAAAGTGTAAATATAACTATTATTTTTATAGT |
| | TGTAATAAAAAGGGAAATTTGATTGTATACCTTCGGTTCTTTAAAAGAAACTGACTTGATAAAAATGGCT |
| | GTAATCTCTAAGGTTACGTATAGTCTATATGATCAAAAAGAGATTAATGCTACAGATATTATCATTAGTC |
| | ATGTTAAAAATGACGACGATATCGGTACCGTTAAAGATGGTAGACTAGGTGCTATGGATGGGGCATTATG |
| | TAAGACTTGTGGGAAAACGGAATTGGAATGTTTCGGTCACTGGGGTAAAGTAAGTATTTATAAAACTCAT |
| | ATAGTTAAGCCTGAATTTATTTCAGAAATTATTCGTTTACTGAATCATATATGTATTCACTGCGGATTAT |
| | TGCGTTCACGAGAACCGTATTCCGACGATATTAACCTAAAAGAGTTATCGGGACACGCTCTTAGGAGATT |
| | AAAGGATAAAATATTATCCAAGAAAAAGTCATGTTGGAACAGTGAATGTATGCAACCGTATCAAAAAATT |
| | ACTTTTTCAAAGAAAAAGGTTTGTTTCGTCAACAAGTTGGATGATATTAACGTTCCTAATTCTCTCATCT |
| | ATCAAAAGTTAATTTCTATTCATGAAAAGTTTTGGCCATTATTAGAAATTCATCAATATCCAGCTAACTT |
| | ATTTTATACAGACTACTTTCCCATCCCTCCGTTGATTATTAGACCGGCTATTAGTTTTTGGATAGATAGT |
| | ATACCCAAAGAAACCAATGAATTAACTTACTTATTAGGTATGATCGTTAAGAATTGTAACTTGAATGCTG |
| | ATGAACAGGTTATCCAGAAGGCGGTAATAGAATACGATGATATTAAAATTATTTCTAATAACACTTCCAG |
| | TATCAATTTATCATATATTACATCCGGCAAAAATAATATGATTAGAAGTTATATCGTCGCCCGACGAAAA |
| | GATCAGACGGCTAGATCTGTAATTGGTCCCAGTACATCTATCACCGTTAATGAGGTAGGAATGCCCGCAT |
| | ATATTAGAAATACACTTACAGAAAAGATATTTGTTAATGCCTTTACAGTGGATAAAGTTAAACAACTATT |
| | AGCATCAAACCAAGTTAAATTTTACTTTAATAAACGATTAAACCAATTAACAAGAATACGCCAAGGAAAG |
| | TTTATCAAAAATAAAATACATTTATTGCCTGGTGATTGGGTAGAAGTAGCTGTTCAAGAATATACAAGTA |
| | TTATTTTTGGAAGACAGCCGTCTCTACATAGATACAACGTCATCGCTTCATCTATCAGAGCTACCGAAGG |
| | AGATACTATCAAAATATCTCCCGGAATTGCCAACTCTCAAAATGCTGATTTCGACGGAGATGAAGAATGG |
| | ATGATATTAGAACAAAATCCTAAAGCTGTAATTGAACAAAGTATTCTTATGTATCCGACGACGTTACTCA |
| | AACACGATATTCATGGAGCCCCCGTTTATGGATCTATTCAAGATGAAATCGTAGCAGCGTATTCATTGTT |
| | TAGGATACAAGATCTTTGTTTAGATGAAGTATTGAACATCTTGGGGAAATATGGAAGAGAGTTCGATCCT |
| | AAAGGTAAATGTAAATTCAGCGGTAAAGATATCTATACTTACTTGATAGGTGAAAAGATTAATTATCCGG |
| | GTCTCTTAAAGGATGGTGAAATTATTGCAAACGACGTAGATAGTAATTTTGTTGTGGCTATGAGGCATCT |
| | GTCATTGGCTGGACTCTTATCCGATCATAAGTCGAACGTGGAAGGTATCAACTTTATTATCAAGTCATCT |
| | TATGTTTTTAAGAGATATCTATCTATTTACGGTTTTGGGGTGACATTCAAAGATCTGAGACCAAATTCGA |
| | CGTTCACTAATAAATTGGAGGCCATCAACGTAGAAAAAATAGAACTTATCAAAGAAGCATACGCCAAATA |
| | TCTCAACGATGTAAGAGACGGGAAAATAGTTCCATTATCTAAAGCTTTAGAGGCGGACTATGTGGAATCC |
| | ATGTTATCCAACTTGACAAATCTTAATATCCGAGAGATAGAAGAACATATGAGACAAACGCTGATAGATG |
| | ATCCAGATAATAACCTCCTGAAAATGGCCAAAGCGGGTTATAAAGTAAATCCTACAGAACTAATGTATAT |
| | TCTAGGTACGTATGGACAACAAAGGATTGATGGTGAACCAGCAGAGACTCGAGTATTGGGTAGAGTCTTA |
| | CCTTACTATCTTCCAGACTCTAAGGATCCAGAAGGAAGAGGTTACATTCTTAATTCTTTAACAAAAGGAT |
| | TAACAGGTTCTCAATATTACTTTTCGATGCTGGTTGCAAGATCTCAATCTACTGATATCGTCTGTGAAAC |
| | ATCACGTACCGGAACACTGGCTAGAAAAATCATTAAAAAGATGGAGGATATGGTGGTCGACGGATACGGA |
| | CAAGTAGTTATAGGTAATACGCTCATCAAGTACGCCGCCAATTATACCAAAATTCTAGGCTCAGTATGTA |
| | AACCTGTAGATCTTATCTATCCAGATGAGTCCATGACTTGGTATTTGGAAATTAGTGCTCTGTGGAATAA |
| | AATAAAACAGGGATTCGTTTACTCTCAGAAACAGAAACTTGCAAAGAAGACATTGGCGCCGTTTAATTTC |
| | CTAGTATTCGTCAAACCCACCACTGAGGATAATGCTATTAAGGTTAAGGATCTGTACGATATGATTCATA |
| | ACGTCATTGATGATGTGAGAGAGAAATACTTCTTTACGGTATCTAATATAGATTTTATGGAGTATATATT |
| | CTTGACGCATCTTAATCCTTCTAGAATTAGAATTACAAAAGAAACGGCTATCACTATCTTTGAAAAGTTC |
| | TATGAAAAACTCAATTATACTCTAGGTGGTGGAACTCCTATTGGAATTATTTCTGCACAGGTATTGTCTG |
| | AGAAGTTTACACAACAAGCCCTGTCCAGTTTTCACACTACTGAAAAAAGTGGTGCCGTCAAACAAAAACT |
| | TGGTTTCAACGAGTTTAATAACTTGACTAATTTGAGTAAGAATAAGACCGAAATTATCACTCTGGTATCC |
| | GATGATATCTCTAAACTTCAATCTGTTAAGATTAATTTCGAATTTGTATGTTTGGGAGAATTAAATCCAG |
| | ACATCACTCTTCGAAAAGAAACAGATAGGTATGTAGTAGATATAATAGTCAATAGATTATACATCAAGAG |
| | AGCAGAAATTACCGAATTAGTCGTCGAATATATGATTGAACGATTCATCTCCTTTAGCGTCATTGTAAAG |
| | GAATGGGGTATGGAAACATTCATTGAGGACGAGGATAATATTAGATTTACTGTCTATCTAAATTTCGTTG |
| | AACCAGAGGAATTGAATCTTAGTAAGTTTATGATGGTTCTTCCGGGGGCAGCCAACAAGGGAAAGATTAG |
| | TAAATTCAAGATTCCTATCTCTGATTATACGGGTTATGACGACTTCAATCAAACAAAAAAGCTCAATAAG |
| | ATGACTGTAGAACTCATGAATCTAAAAGAATTAGGTTCTTTCGATTTGGAAAACGTCAACGTGTATCCTG |
| | GAGTATGGAATACATACGATATCTTCGGTATCGAGGCCGCTCGTGAATACTTGTGCGAAGCCATGTTAAA |
| | CACCTATGGAGAAGGGTTCGATTATCTGTATCAGCCTTGTGATCTTCTCGCTAGTTTACTATGTGCTAGT |
| | TACGAACCAGAATCAGTGAATAAATTCAAGTTCGGCGCAGCTAGTACTCTTAAGAGAGCTACGTTCGGAG |
| | ACAATAAAGCATTGTTAAACGCGGCTCTTCATAAAAAGTCAGAACCTATTAACGATAATAGTAGCTGCCA |
| | CTTTTTTAGCAAGGTCCCTAATATAGGAACTGGATATTACAAATACTTTATCGACTTGGGTCTTCTCATG |
| | AGAATGGAAAGGAAACTATCTGATAAGATATCTTCTCAAAAGATCAAGGAAATGGAAGAAACAGAAGACT |
| | TTTAATTCTTATCAATAACATATTTTTCTATGATCTGTCTTTTAAACGATGGATTTTCCACAAATGCGCC |
| | TCTCAAGTCCCTCATAGAATGATACACGTATAAAAAATATAGCATAGGCAATGACTCCTTATTTTTAGAC |
| | ATTAGATATGCCAAAATCATAGCCCCGCTTCTATTTACTCCCGCAGCACAATGAACCAACACGGGCTCGT |
| | TTCGTTGATCACATTTAGATAAAAAGGCGGTTACGTCGTCAAAATATTTACTAATATCGGTAGTTGTATC |
| | ATCTACCAACGGTATATGAATAATATTAATATTAGAGTTAGGTAATGTATATTTATCCATCGTCAAATTT |
| | AAAACATATTTGAACTTAACTTCAGATGATGGTGCATCCATAGCATTTTTATAATTTCCCAAATACACAT |
| | TATTGGTTACCCTTGTCATTATAGTGGGAGATTTGGCTCTGTGCATATCTCCAGTTGAACGTAGTAGTAA |
| | GTATTTATACAAACTTTTCTTATCCATTTATAACGTACAAATGGATAAAACTACTTTATCGGTAAACGCG |
| | TGTAATTTAGAATACGTTAGAGAAAAGGCTATAGTAGGCGTACAAGCAGCCAAAACATCAACACTTATAT |
| | TCTTTGTTATTATATTGGCAATTAGTGCGCTATTACTCTGGTTTCAGACGTCTGATAATCCAGTCTTTAA |
| | TGAATTAACGAGATATATGCGAATTAAAAATACGGTTAACGATTGGAAATCATTAACGGATAGCAAAACA |
| | AAATTAGAAAGTGATAGAGGTAGACTTCTAGCCGCTGGTAAGGATGATATATTCGAATTCAAATGTGTGG |
| | ATTTCGGCGCCTATTTTATAGCTATGCGATTGGATAAGAAAACATATCTGCCGCAAGCTATTAGGCGAGG |
| | TACTGGAGACGCGTGGATGGTTAAAAAGGCGGCAAAGGTCGATCCATCTGCTCAACAATTTTGTCAGTAT |
| | TTGATAAAACACAAGTCTAATAATGTTATTACTTGTGGTAATGAGATGTTAAATGAATTAGGTTATAGCG |
| | GTTATTTTATGTCACCGCATTGGTGTTCCGATTTTAGTAATATGGAATAGTGTTAGATAAATGCGGTAAC |
| | GAATGTTCCTGTAAGGAACCATAACAGTTTAGATTTAACGTTAAAGATGAGCATAAACATAATAAACAAA |
| | ATTACAATCAAACCTATAACATTAATATCAAACAATCCAAAAAATGAAATCAGTGGAGTAGTAAACGCGT |
| | ACATAACTCCTGGATAACGTTTAGTAGCTGCCGTTCCTATTCTAGACCAAAAATTCGGTTTCATGTTTTC |
| | GAAACGGTGTTCTGCAACAAGTCGGGGATCGTGTTCTACATATTTGGCGGCATTATCCAGTATCTGCCTA |
| | TTGATCTTCATTTCGTTTTCAATTCTGGCTATTTCAAAATAAAATCCCGATGATAGACCTCCAGACTTTA |
| | TAATTTCATCTACGATGTTCAGCGCCGTAGTAACTCTAATAATATAGGCTGATAAGCTAACATCATACCC |
| | TCCTGTATATGTGAATATGGCATGATTTTTGTCCATTACAAGCTCGGTTTTAACTTTATTGCCTGTAATA |
| | ATTTCTCTCATCTGTAGGATATCTATTTTTTTGTCATGCATTGCCTTCAAGACGGGACGAAGAAACGTAA |
| | TATCCTCAATAACGTTATCGTTTTCTACAATAACTACATATTCTACCTTTTTATTTTCTAACTCGGTAAA |
| | AAAATTAGAATCCCATAGGGCTAAATGTCTAGCGATATTTCTTTTCGTTTCCTCTGTACACATAGTGTTA |
| | CAAAACCCTGAAAAGAAGTGAGTATACTTGTCATCATTTCTAATGTTTCCTCCAGTCCACTGTATAAACG |
| | CATAATCCTTGTAATGATCTGGATCATCCTTGACTACCACAACATTTCTTTTTTCTGGCATAACTTCGTT |
| | GTCCTTTACATCATCGAACTTCTGATCATTAATATGCTCATGAACATTAGGAAATGTTTCTGATGGAAGT |
| | CTATCAATAACTGGCACAACAATAACAGGAGTTTTCGCCGCCGCCATTTAGTTATTGAAATTAATCATAT |
| | ACAACTCTTTAATACGAGTTATATTTTCGTCTATCCATTGTTTCACATTTACATATTTCGACAAAAAGAT |
| | ATAAAATGCGTATTCCAATGCTTCTCTGTTTAATGAATTACTAAAATATACAAACACGTCACTGTCTGGC |
| | AATAAATGATATCTTAGAATATTGTAACAATTTATTTTGTATTGCACATGTTCGTGATCTATGAGTTCTT |
| | CTTCGAATGGCATAGGATCTCCGAATCTGAAAACGTATAAATAGGAGTTAGAATAATAATATTTGAGAGT |
| | ATTGGTAATATATAAACTCTTTAGCGGTATAATTAGTTTTTTTCTCTCAATTTCTATTTTTAGATGTGAT |
| | GGAAAAATGACTAATTTTGTAGCATTAGTATCATGAACTCTAATCAAAATCTTAATATCTTCGTCACACG |
| | TTAGCTCTTTGAAGTTTTTAAGAGATGCATCAGTTGGTTCGACCGATGGAGTAGGTGCAACAATTTTTTG |
| | TTCGATGTATGTATGTACTGGAGCCATTGTTTTAACTATAATGGTGCTTGTATCGAAAAACTTTAATGCA |
| | GATAGCGGAAGCTCTTCGCCGCGACTTTCTACATCGTAATTGGGTTCTAACGCCGATCTCTGAATGGATA |
| | CTAGTTTTCTAAGTTCTAATGTGATTCTCTGAAAATGTAAATCCAATTCCTCCGGCATTATAGATGTGTA |
| | TACATCGGTAAATAAAACTATAGTATCCAACGATCCCTTCTCGCAAATTCTAGTCTTAACCAAAAAATCG |
| | TATATAACCACGGAGATGGCGTATTTAAGAGTGGATTCTTCTACCGTTTTGTTCTTGGATGTCATATAGG |
| | AAACTATAAAGTCCGCACTACTGTTAAGAATGATTACTAACGCAACTATATAGTTCAAATTAAGCATTTT |
| | GGAAACATAAAATAACTCTGTAGACGATACTTGACTTTCGAATAAGTTTGCAGACAAACGAAGAAAGAAC |
| | AGACCTCTCTTAATTTCAGAAGAAAACTTTTTTTCGTATTCCTGACGTCTAGAGTTTATATCAATAAGAA |
| | AGTTAAGAATTAGTCGGTTAATGTTGTATTTCATTACCCAAGTTTGAGATTTCATAATATTATCAAAAGA |
| | CATGATAATATTAAAGATAAAGCGCTGACTATGAACGAAATAGCTATATGGTTCGCTCAAAAATATAGTC |
| | TTGTTAAACGTGGAAACGATAACTGTATTTTTAATCACGTCAGCGGCATCTAAATTAAATATAGGTATAT |
| | TTATTCCACACACTCTACAATATGCCACACCATCTTCATAATAAATAAATTCGTTAGCAAAATTATTAAT |
| | TTTAGTGAAATAGTTAGCGTCAACTTTCATAGCTTCCTTCAATCTAATTTGATGCTCACACGGTGCGAAT |
| | TCCACTCTAACATCCCTTTTCCATGCCTCAGGTTCATCGATCTCTATAATATCTAGTTTTTTGCGTTTCA |
| | CAAACACAGGCTCGTCTCTCGCGATGAGATCTGTATAGTAACTATGTAAATGATAACTAGATAGAAAGAT |
| | GTAGCTATATAGATGACGATCCTTTAAGAGAGGTATAATAACTTTACCCCAATCAGATAGACTGTTGTTA |
| | TGGTCTTCGGAAAAAGAATTTTTATAAATTTTTCCAGTATTTTCCAAATATACGTACTTAACATCTAAAA |
| | AATCCTTAATGATAATAGGAATGGATAATCCGTCTATTTTATAAAGAAATACATATCGCACATTATACTT |
| | TTTTTTGGAAATGGGAATACCGATGTGTCTACATAAATATGCAAAGTCTAAATATTTTTTAGAGAATCTT |
| | AATTGGTCCAAATTCTTTTCCAAGTACGGTAATAGATTTTTCATATTGAACGGTATCTTCTTAATCTCTG |
| | GTTCTAGTTCCGCATTAAATGATGAAACTAAGTCACTATTTTTATAACTAACGATTACATCACCTCTAAC |
| | ATCATCATTTACCAGAATACTGATCTTCTTTTGTCGTAAATACATGTCTAATGTGTTAAAAAAAAGATCA |
| | TACAAGTTATACGTCATTTCATCTGTGGTATTCTTGTCATTGAAGGATAAACTCGTACTAATCTCTTCTT |
| | TAACAGCCTGTTCAAATTTATATCCTATATACGAAAAAATAGCAACCAGTGTTTGATCATCCGCGTCAAT |
| | ATTCTGTTCTATCGTAGTGTATAACAATCGTATATCTTCTTCTGTGATAGTCGATACGTTATAAAGGTTG |
| | ATAACGAAAATATTTTTATTTCGTGAAATAAAGTCATCGTAGGATTTTGGACTTATATTCGCGTCTAGTA |
| | GATATGCTTTTATTTTTGGAATGATCTCAATTAGAATAGTCTCTTTAGAGTCCATTTAAAGTTACAAACA |
| | ACTAGGAAATTGGTTTATGATGTATAATTTTTTTAGTTTTTATAGATTCTTTATTCTATACTTAAAAAAT |
| | GAAAATAAATACAAAGGTTCTTGAGGGTTGTGTTAAATTGAAAGCGAGAAATAATCATAAATTATTTCAT |
| | TATCGCGATATCCGTTAAGTTTGTATCGTAATGGCGTGGTCAATTACAAATAAAGCGGATACTAGTAGCT |
| | TCACAAAGATGGCTGAAATCAGAGCTCATCTAAAAAATAGCGCTGAAAATAAAGATAAAAACGAGGATAT |
| | TTTCCCGGAAGATGTAATAATTCCATCTACTAAGCCCAAAACCAAACGAGCCACTACTCCTCGTAAACCA |
| | GCGGCTACTAAAAGATCAACCAAAAAGGAGGAAGTGGAAGAAGAAGTAGTTATAGAGGAATATCATCAAA |
| | CAACTGAAAAAAATTCTCCATCTCCTGGAGTCAGCGACATTGTAGAAAGCGTGGCCGCTGTAGAGCTCGA |
| | TGATAGCGACGGGGATGATGAACCTATGGTACAAGTTGAAGCTGGTAAAGTAAATCATAGTGCTAGAAGC |
| | GATCTCTCTGACCTAAAGGTGGCTACCGACAATATCGTTAAAGATCTTAAGAAAATTATTACTAGAATCT |
| | CTGCAGTATCGACGGTTCTAGAGGATGTTCAAGCAGCTGGTATCTCTAGACAATTTACTTCTATGACTAA |
| | AGCTATTACAACACTATCTGATCTAGTCACCGAGGGAAAATCTAAAGTTGTTCGTAAAAAAGTTAAAACT |
| | TGTAAGAAGTAAATGCGTGCACTTTTTTATAAAGATGGTAAACTCTTTACCGATAATAATTTTTTAAATC |
| | CTGTATCAGACGATAATCCAGCGTATGAGGTTTTGCAACATGTTAAAATTCCTACTCATTTAACAGATGT |
| | AGTAGTATATGAACAAACGTGGGAGGAGGCGTTAACTAGATTAATTTTTGTGGGAAGTGATTCAAAAGGA |
| | CGTAGACAATACTTTTACGGAAAAATGCATGTACAGAATCGCAACGCTAAAAGAGATCGTATTTTTGTTA |
| | GAGTATATAACGTTATGAAACGAATTAATTGTTTTATAAACAAAAATATAAAGAAATCGTCCACAGATTC |
| | CAATTATCAGTTGGCGGTTTTTATGTTAATGGAAACTATGTTTTTTATTAGATTTGGTAAAATGAAATAT |
| | CTTAAGGAGAATGAAACAGTAGGGTTATTAACACTAAAAAATAAACACATAGAAATAAGTCCCGATGAAA |
| | TAGTTATCAAGTTTGTAGGAAAGGACAAAGTTTCACATGAATTTGTTGTTCATAAGTCTAATAGACTATA |
| | TAAGCCGCTATTGAAACTGACGGATGATTCTAGTCCCGAAGAATTTCTGTTCAACAAACTAAGTGAACGA |
| | AAGGTATATGAATGTATCAAACAGTTTGGTATTAGAATCAAGGATCTCCGAACGTATGGAGTCAATTATA |
| | CGTTTTTATATAATTTTTGGACAAATGTAAAGTCCATATCTCCTCTTCCATCACCAAAAAAGTTAATAGC |
| | GTTAACTATCAAACAAACTGCTGAAGTGGTAGGTCATACTCCATCAATTTCAAAAAGAGCTTATATGGCA |
| | ACGACTATTTTAGAAATGGTAAAGGATAAAAATTTTTTAGATGTAGTATCTAAAACTACGTTCGATGAAT |
| | TCCTATCTATAGTCGTAGATCACGTTAAATCATCTACGGATGGATGATATAGATCTTTACACAAATAATT |
| | ACAAAACCGATAAATGGAAATGGATAAGCGTATGAAATCTCTCGCAATGACCGCTTTCTTTGGGGAGCTA |
| | AGCACATTAGATATTATGGCATTGATAATGTCTATATTTAAACGCCATCCAAACAATACCATTTTTTCAG |
| | TGGATAAGGATGGTCAGTTTATGATTGATTTCGAATACGATAATTATAAGGCTTCTCAATATTTGGATCT |
| | GACCCTCACTCCGATATCTGGAGATGAATGCAAGACTCACGCATCGAGTATAGCCGAACAATTGGCGTGT |
| | GTGGATATTATTAAAGAGGATATTAGCGAATATATCAAAACTACTCCCCGTCTTAAACGATTTATAAAAA |
| | AATACCGCAATAGATCAGATACTCGCATCAGTCGAGATACAGAAAAGCTTAAAATAGCTCTAGCTAAAGG |
| | CATAGATTACGAATATATAAAAGACGCTTGTTAATAAGTAAATGAAAAAAAACTAGTCGTTTATAATAAA |
| | ACACGATATGGATGCCAACGTAGTATCATCTTCTACTATTGCGACGTATATAGACGCTTTAGCGAAGAAT |
| | GCTTCGGAATTAGAACAGAGGTCTACCGCATACGAAATAAATAATGAATTGGAACTAGTATTTATTAAGC |
| | CGCCATTGATTACTTTGACAAATGTAGTGAATATCTCTACGATTCAGGAATCGTTTATTCGATTTACCGT |
| | TACTAATAAGGAAGGTGTTAAAATTAGAACTAAGATTCCATTATCTAAGGTACATGGTCTAGATGTAAAA |
| | AATGTACAGTTAGTAGATGCTATAGATAACATAGTTTGGGAAAAGAAATCATTAGTGACGGAAAATCGTC |
| | TTCACAAAGAATGCTTGTTGAGACTATCGACAGAGGAACGTCATATATTTTTGGATTACAAGAAATATGG |
| | ATCCTCTATCCGACTAGAATTAGTCAATCTTATTCAAGCAAAAACAAAAAACTTTACGATAGACTTTAAG |
| | CTAAAATATTTTCTAGGATCCGGTGCCCAGTCTAAAAGTTCTTTATTACACGCTATTAATCATCCAAAGT |
| | CAAGGCCTAATACATCTCTGGAAATAGAATTTACACCTAGAGACAATGAAACAGTTCCATATGATGAACT |
| | AATAAAGGAATTGACGACTCTCTCGCGTCATATATTTATGGCTTCTCCAGAGAATGTAATTCTTTCTCCG |
| | CCTATTAACGCGCCTATAAAAACCTTTATGTTGCCTAAACAAGATATAGTAGGTTTGGATCTGGAAAATC |
| | TATATGCCGTAACTAAGACTGACGGCATTCCTATAACTATCAGAGTTACATCAAACGGGTTGTATTGTTA |
| | TTTTACACATCTTGGTTATATTATTAGATATCCTGTTAAGAGAATAATAGATTCCGAAGTAGTAGTCTTT |
| | GGTGAGGCAGTTAAGGATAAGAACTGGACCGTATATCTCATTAAGCTAATAGAGCCTGTGAATGCAATCA |
| | ATGATAGACTAGAAGAAAGTAAGTATGTTGAATCTAAACTAGTGGATATTTGTGATCGGATAGTATTCAA |
| | GTCAAAGAAATACGAAGGTCCGTTTACTACAACTAGTGAAGTCGTCGATATGTTATCTACATATTTACCA |
| | AAGCAACCAGAAGGTGTTATTCTGTTCTATTCAAAGGGACCTAAATCTAACATTGATTTTAAAATTAAAA |
| | AGGAAAATACTATAGACCAAACTGCAAATGTAGTATTTAGGTACATGTCCAGTGAACCAATTATCTTTGG |
| | AGAGTCGTCTATCTTTGTAGAGTATAAGAAATTTAGCAACGATAAAGGCTTTCCTAAAGAATATGGTTCT |
| | GGTAAGATTGTGTTATATAACGGCGTTAATTATCTAAATAATATCTATTGTTTGGAATATATTAATACAC |
| | ATAATGAAGTGGGTATTAAGTCCGTGGTTGTACCTATTAAGTTTATAGCAGAATTCTTAGTTAATGGAGA |
| | AATACTTAAACCTAGAATTGATAAAACCATGAAATATATTAACTCAGAAGATTATTATGGAAATCAACAT |
| | AATATCATAGTCGAACATTTAAGAGATCAAAGCATCAAAATAGGAGATATCTTTAACGAGGATAAACTAT |
| | CGGATGTGGGACATCAATACGCCAATAATGATAAATTTAGATTAAATCCAGAAGTTAGTTATTTTACGAA |
| | TAAACGAACTAGAGGACCGTTGGGAATTTTATCAAACTACGTCAAGACTCTTCTTATTTCTATGTATTGT |
| | TCCAAAACATTTTTAGACGATTCCAACAAACGAAAGGTATTGGCGATTGATTTTGGAAACGGTGCTGACC |
| | TGGAAAAATACTTTTATGGAGAGATTGCGTTATTGGTAGCGACGGATCCGGATGCTGATGCTATAGCTAG |
| | AGGAAATGAAAGATACAACAAATTAAACTCTGGAATTAAAACCAAGTACTACAAATTTGACTACATTCAG |
| | GAAACTATTCGATCCGATACATTTGTCTCTAGTGTCAGAGAAGTATTCTATTTTGGAAAGTTTAATATCA |
| | TCGACTGGCAGTTTGCTATCCATTATTCTTTTCATCCGAGACATTATGCTACCGTCATGAATAACTTATC |
| | CGAACTAACTGCTTCTGGAGGCAAGGTATTAATCACTACCATGGACGGAGACAAATTATCAAAATTAACA |
| | GATAAAAAGACTTTTATAATTCATAAGAATTTACCTAGTAGCGAAAACTATATGTCTGTAGAAAAAATAG |
| | CTGATGATAGAATAGTGGTATATAATCCATCAACAATGTCTACTCCAATGACTGAATACATTATCAAAAA |
| | GAACGATATAGTCAGAGTGTTTAACGAATACGGATTTGTTCTTGTAGATAACGTTGATTTCGCTACAATT |
| | ATAGAACGAAGTAAAAAGTTTATTAATGGCGCATCTACAATGGAAGATAGACCATCTACAAGAAACTTTT |
| | TCGAACTAAATAGAGGAGCCATTAAATGTGAAGGTTTAGATGTCGAAGACTTACTTAGTTACTATGTTGT |
| | TTATGTCTTTTCTAAGCGGTAAATAATAATATGGTATGGGTTCTGATCTCCCAGTTCTAAATGCATTAAA |
| | TAATTCCAATAGAGCGATTTTTGTTCCTATAGGACCTTCCAACTGTGGATACTCTGTATTGTTAATAGAT |
| | ATATTAATACTTTTGTCGGGTAACAGAGGTTCTACGTCTTTTAAAAATAAAAGTTTGATAACATCTGGCC |
| | TGTTCATAAATAAAAACTTGGCGATTCTATATATACTCTTATTATCAAATCTAGCCATTGTCTTATAGAT |
| | GTGAGCTACTGTAGGTGTACCATTTGATTTTCTTTCTAATACTATATATTTCTCTCGAAGAAGTTCTTGC |
| | ACATCATCTGGGAATAAAATACTACTGTTGAGTAAATCAGTTATTTTTTTTATATCGATATTGATGGACA |
| | TTTTTATAGTTAAGGATAATAAGTATCCCAAAGTAGATAACGACGATAACGAAGTATTTATACTTTTAGG |
| | AAATCACAATGACTTTATCAGATTAAAATTAACAAAATTAAAGGAGCATGTATTTTTTTCTGAATATATT |
| | GTGACTCCAGATACATATGGATCTTTATGCGTCGAATTAAATGGGTCTAGTTTTCAGCACGGCGGTAGAT |
| | ATATAGAGGTGGAGGAATTTATAGATGCTGGAAGACAAGTTAGATGGTGTTCTACATCCAATCATATATC |
| | TAAAGATATACCCGAAGATATGCACACTGATAAATTTGTCATTTATGATATATACACTTTTGACGCTTTC |
| | AAGAATAAACGATTGGTATTCGTACAGGTACCTCCGTCGTTAGGAGATGATAGTCATTTGACTAATCCGT |
| | TATTGTCTCCGTATTATCGTAATTCAGTAGCCAGACAAATGGTCAATGATATGATTTTTAATCAAGATTC |
| | ATTTTTAAAATATTTATTAGAACATCTGATTAGAAGCCACTATAGAGTTTCTAAACATATAACAATAGTT |
| | AGATACAAGGATACCGAAGAATTAAATCTAACGAGAATATGTTATAATAGAGATAAGTTTAAGGCGTTTG |
| | TATTCGCTTGGTTTAACGGCGTTTCGGAAAATGAAAAGGTACTAGATACGTATAAAAAGGTATCTAATTT |
| | GATATAATGAATTCAGTGACTGTATCACACGCGCCATATACTATTACTTATCACGATGATTGGGAACCAG |
| | TAATGAGTCAATTGGTAGAGTTTTATAACGAAGTAGCCAGTTGGCTGCTACGAGACGAGACGTCGCCTAT |
| | TCCTGATAAGTTCTTTATACAGTTGAAACAACCGCTTAGAAATAAACGAGTATGTGTGTGCGGTATAGAT |
| | CCGTATCCGAAAGATGGAACTGGTGTACCGTTCGAATCACCAAATTTTACAAAAAAATCAATTAAGGAGA |
| | TAGCTTCATCTATATCTAGATTAACCGGAGTAATTGATTATAAAGGTTATAACCTTAATATAATAGACGG |
| | GGTTATACCCTGGAATTATTACTTAAGTTGTAAATTAGGAGAAACAAAAAGTCACGCGATCTACTGGGAT |
| | AAGATTTCCAAGTTACTGCTGCAGCATATAACTAAACACGTTAGTGTTCTTTATTGTTTGGGTAAAACAG |
| | ATTTCTCGAATATACGGGCCAAGTTAGAATCCCCGGTAACTACCATAGTCGGATATCATCCAGCGGCTAG |
| | AGACCGCCAATTCGAGAAAGATAGATCATTTGAAATTATCAACGTTTTACTGGAATTAGACAACAAGGCA |
| | CCTATAAATTGGGCTCAAGGGTTTATTTATTAATGCTTTAGTGAAATTTTAACTTGTGTTCTAAATGGAT |
| | GCGGCTATTAGAGGTAATGATGTTATCTTTGTTCTTAAGACTATAGGTGTCCCGTCAGCGTGCAGACAAA |
| | ATGAAGATCCAAGATTTGTAGAAGCATTTAAATGCGACGAGTTAGAAAGATATATTGAGAATAATCCAGA |
| | ATGTACACTATTCGAAAGTCTTAGGGATGAGGAAGCATACTCTATAGTCAGAATTTTCATGGATGTAGAT |
| | TTAGACGCGTGTCTAGACGAAATAGATTATTTAACGGCTATTCAAGATTTTATTATCGAGGTGTCAAACT |
| | GTGTAGCTAGATTCGCGTTTACAGAATGCGGCGCCATTCATGAAAATGTAATAAAATCCATGAGATCTAA |
| | TTTTTCATTGACTAAGTCTACAAATAGAGATAAAACAAGTTTTCATATTATCTTTTTAGACACGTATACC |
| | ACTATGGATACATTGATAGCTATGAAACGAACACTATTAGAATTAAGTAGATCATCTGAAAATCCACTAA |
| | CAAGATCGATAGACACTGCCGTATATAGGAGAAAAACAACTCTTCGGGTTGTAGGTACTAGGAAAAATCC |
| | AAATTGCGACACTATTCATGTAATGCAACCACCGCATGATAATATAGAAGATTACCTATTCACTTACGTG |
| | GATATGAACAACAATAGTTATTACTTTTCTCTACAACAACGATTGGAGGATTTAGTTCCTGATAAGTTAT |
| | GGGAACCAGGGTTTATTTCATTCGAAGACGCTATAAAAAGAGTTTCAAAAATATTCATTAATTCTATAAT |
| | AAACTTTAATGATCTCGATGAAAATAATTTTACAACGGTACCACTGGTCATAGATTACGTAACACCTTGT |
| | GCATTATGTAAAAAACGATCGCATAAACATCCGCATCAACTATCGTTGGAAAATGGTGCTATTAGAATTT |
| | ACAAAACTGGTAATCCACATAGTTGTAAAGTTAAAATTGTTCCGTTGGATGGTAATAAACTGTTTAATAT |
| | TGCACAAAGAATTTTAGACACTAACTCTGTTTTATTAACCGAACGAGGAGACCATATAGTTTGGATTAAT |
| | AATTCATGGAAATTTAACAGCGAAGAACCCTTGATAACAAAACTAATTTTGTCAATAAGACATCAACTAC |
| | CTAAGGAATATTCAAGCGAATTACTCTGTCCAAGAAAACGAAAGACTGTAGAAGCTAACATACGAGACAT |
| | GTTAGTAGATTCAGTAGAGACCGATACCTATCCGGATAAACTTCCGTTTAAAAATGGTGTATTGGACCTG |
| | GTAGACGGAATGTTTTACTCTGGAGATGATGCTAAAAAATATACGTGTACTGTATCAACCGGATTTAAAT |
| | TTGACGATACAAAGTTCGTCGAAGACAGTCCAGAAATGGAAGAGTTAATGAATATCATTAACGATATCCA |
| | ACCATTAACGGATGAAAATAAGAAAAATAGAGAGCTATATGAAAAAACATTATCTAGTTGTTTATGCGGT |
| | GCTACCAAAGGATGTTTAACATTCTTTTTTGGAGAAACTGCAACTGGAAAGTCGACAACCAAACGTTTGT |
| | TAAAGTCTGCTATCGGTGACCTGTTTGTTGAGACGGGTCAAACAATTTTAACAGATGTATTGGATAAAGG |
| | ACCTAATCCATTTATCGCTAACATGCATTTGAAAAGATCTGTATTCTGTAGCGAACTACCTGATTTTGCC |
| | TGTAGTGGATCAAAGAAAATTAGATCTGACAATATTAAAAAGTTGACAGAACCTTGTGTCATTGGAAGAC |
| | CGTGTTTCTCCAATAAAATTAATAATAGAAACCATGCGACAATCATTATCGATACTAATTACAAACCTGT |
| | TTTTGATAGGATAGATAACGCATTAATGAGAAGAATTGCCGTCGTGCGATTCAGAACACACTTTTCTCAA |
| | CCTTCTGGTAGAGAGGCTGCTGAAAATAATGACGCGTACGATAAAGTCAAACTATTAGACGAGGGGTTAG |
| | ATGGTAAAATACAAAATAATAGATATAGATTCGCATTTCTATACTTGTTGGTGAAATGGTACAGAAAATA |
| | TCATGTTCCTATTATGAAACTATATCCTACACCCGAAGAGATTCCTGACTTTGCATTCTATCTCAAAATA |
| | GGTACTCTGTTAGTATCTAGCTCTGTAAAGCATATTCCATTAATGACGGACCTCTCCAAAAAGGGATATA |
| | TATTGTACGATAATGTGGTCACTCTTCCGTTGACTACTTTCCAACAGAAAATATCCAAGTATTTTAATTC |
| | TAGACTATTTGGACACGATATAGAGAGCTTCATCAATAGACATAAGAAATTTGCCAATGTTAGTGATGAA |
| | TATCTGCAATATATATTCATAGAGGATATTTCATCTCCGTAAATATATGCTCATATATTTATAGAAGATA |
| | TCACATATCTAAATGAATACCGGAATCATAGATTTATTTGATAATCATGTTGATAGTATACCAACTATAT |
| | TACCTCATCAGTTAGCTACTCTAGATTATCTAGTTAGAACTATCATAGATGAGAACAGAAGCGTGTTATT |
| | GTTCCATATTATGGGATCAGGTAAAACAATAATCGCTTTGTTGTTCGCCTTGGTAGCTTCCAGATTTAAA |
| | AAGGTTTACATTCTAGTGCCTAATATCAACATTTTGAAAATTTTTAATTATAATATGGGTGTAGCTATGA |
| | ACTTGTTTAATGACGAATTCATAGCTGAGAATATCTTTATTCATTCCACAACAAGTTTTTATTCTCTTAA |
| | TTATAACGATAACGTCATTAATTATAACGGATTATCTCGCTACAATAACTCTATTTTTATCGTTGATGAG |
| | GCACATAATATCTTTGGGAATAATACTGGAGAACTTATGACCGTGATAAAAAATAAAAACAAGATTCCTT |
| | TTCTACTATTGTCTGGATCTCCCATTACTAACACACCTAATACTCTGGGTCATATTATAGATTTAATGTC |
| | CGAAGAGACGATAGATTTTGGTGAGATTATTAGTCGTGGTAAGAAAGTAATTCAGACACTTCTTAACGAA |
| | CGCGGTGTGAATGTACTTAAGGATTTGCTTAAAGGAAGAATATCATATTACGAAATGCCTGATAAAGATC |
| | TACCAACGATAAGATATCACGGACGTAAGTTTCTAGATACTAGAGTAGTATATTGTCACATGTCTAAACT |
| | TCAAGAGAGAGATTATATGATTACTAGACGACAGCTATGTTATCATGAAATGTTTGATAAAAATATGTAT |
| | AACGTGTCAATGGCAGTATTGGGACAACTTAATCTGATGAATAATTTAGATACTTTATTTCAGGAACAGG |
| | ATAAGGAATTGTACCCAAATCTGAAAATAAATAATGGCGTGTTATACGGAGAAGAATTGGTAACGTTAAA |
| | CATTAGTTCCAAATTTAAATACTTTATTAATCGGATACAGACACTCAACGGAAAACATTTTATATACTTT |
| | TCTAATTCTACATATGGCGGATTGGTAATTAAATATATCATGCTCAGTAATGGATATTCTGAATATAATG |
| | GTTCTCAGGGAACTAATCCACATATGATAAACGGCAAACCAAAAACATTTGCTATCGTTACTAGTAAAAT |
| | GAAATCGTCTTTAGAGGATCTATTAGATGTGTATAATTCTCCTGAAAACGATGATGGTAGTCAATTGATG |
| | TTTTTGTTTTCGTCAAACATTATGTCCGAATCCTATACTCTGAAAGAGGTAAGGCATATTTGGTTTATGA |
| | CTATCCCAGATACTTTTTCTCAATACAACCAAATTCTTGGACGATCTATTAGAAAATTCTCTTACGCCGA |
| | TATTTCTGAACCAGTTAATGTATATCTTTTAGCCGCCGTATATTCCGATTTCAATGACGAAGTAACGTCA |
| | TTAAACGATTACACACAGGATGAATTGATTAATGTTTTACCATTTGACATCAAAAAGCTGTTATATCTAA |
| | AATTTAAGACTAAAGAAACGAATAGAATATACTCTATTCTTCAAGAGATGTCTGAAACGTATTCTCTTCC |
| | ACCACATCCATCAATTGTAAAAGTTTTATTGGGAGAATTGGTCAGACAATTTTTTTATAATAATTCTCGT |
| | ATTAAGTATAACGACTCCAAGTTACTTAAAATGGTTACATCAGTTATAAAAAATAAAGAAGACGCTAGGA |
| | ATTACATAGATGATATTGTAAACGGTCACTTCTTTGTATCGAATAAAGTATTTGATAAATCTCTTTTATA |
| | CAAATACGAAAACGATATTATTACAGTACCGTTTAGACTTTCCTACGAACCATTTGTTTGGGGAGTTAAC |
| | TTTCGTAAAGAATATAACGTGGTATCTTCTCCATAAAACTGATGAAATATATAAAGAAATAAATGTCGAG |
| | CTTTGTTACCAATGGATACCTTCCAGTTACATTGGAACCACACGAGCTGACGTTAGACATAAAAACTAAT |
| | ATTAGGAATGCCGTATATAAGACGTATCTCCATAGAGAAATTAGTGGTAAAATGGCCAAGAAAATAGAAA |
| | TTCGTGAAGACGTGGAATTACCTCTCGGCGAAATAGTTAATAATTCTGTAGTTATAAACGTTCCGTGTGT |
| | AATAACCTACGCGTATTATCACGTTGGGGATATAGTCAGAGGAACATTAAACATCGAAGATGAATCAAAT |
| | GTAACTATTCAATGTGGAGATTTAATCTGTAAACTAAGTAGAGATTCGGGTACTGTATCATTTAGCGATT |
| | CAAAGTACTGCTTTTTTCGAAATGGTAATGCGTATGACAATGGCAGCGAAGTCACTGCCGTTCTAATGGA |
| | GGCTCAACAAGGTATCGAATCTAGTTTTGTTTTTCTCGCGAATATCGTCGACTCATAAAAAAGAGAATAG |
| | CGGTAAGTATAAACACGAATACTATGGCAATAATTGCGAATGTTTTATTCTCTTCGATATATTTTTGATA |
| | ATATGAAAAACATGTCTCTCTCAAATCGGACAACCATCTCATAAAATAGTTCTCGCGCGCTGGAGAGGTA |
| | GTTGCTGCTCGTATAATCTCCCCAGAATAATATACTTGCGTGTCGTCGTTCAATTTATACGGATTTCTAT |
| | AGTTCTCTGTTATATAATGCGGTTTTCCATCATGATTAGACGACGACAATAGTGTTCTGAATTTAGATAG |
| | TTGATCAGAATGAATGTTTATTGGCGTTGGAAAAATTATCCATACAGCGTCTGCAGAGTGGTTGATAGTT |
| | GTTCCTAGATATGTAAAATAATCCAACTTACTAGGCAGCAAATTGTCTAGATAAAATACTGAATCAAACG |
| | GTGCAGACGTATTGGCGGATCTAATGGAATCCAATTGATTAACTATCTTTTGAAAATATACATTTTTATG |
| | ATCCAATACTTGTAAGAATATAGAAATAATGATAAGTCCATCATCGTGTTTTTTTGCCTCTTCATAAGAA |
| | CTATATTTTTTCTTATTCCAATGAACAAGATTAATCTCTCCAGAGTATTTGTACACATCTATCAAGTGAT |
| | TGGATCCATAATCGTCTTCCTTTCCCCAATATATATGTAGTGATGATAACACATATTCATTGGGGAGAAA |
| | CCCTCCACTTATATATCCTCCTTTAAAATTAATCCTTACTAGTTTTCCAGTGTTCTGGATAGTGGTTGGT |
| | TTCGACTCATTATAATGTATGTCTAACGGCTTCAATCGCGCGTTAGAAATTGCTTTTTTAGTTTCTATAT |
| | TAATAGGAGATAGTTGTTGCGGCATAGTAAAAATGAAATGATAACTGTTTAAAAATAGCTCTTAGTATGG |
| | GAATTACAATGGATGAGGAAGTGATATTTGAAACTCCTAGAGAATTAATATCTATTAAACGAATAAAAGA |
| | TATTCCAAGATCAAAAGACACGCACGTGTTTGCTGCGTGTATAACAAGTGACGGATATCCGTTAATAGGA |
| | GCTAGAAGAACTTCATTCGCGTTCCAGGCGATATTATCTCAACAAAATTCAGATTCTATCTTTAGAGTAT |
| | CCACTAAACTATTACGGTTTATGTACTACAATGAACTAAGAGAAATCTTTAGACGGTTGAGAAAAGGTTC |
| | TATCAACAATATCGATCCTCACTTTGAAGAGTTAATATTATTGGGTGGTAAACTAGATAAAAAGGAATCT |
| | ATTAAAGATTGTTTAAGAAGAGAATTAAAAGAGGAAAGTGATGAACGTATAACAGTAAAAGAATTCGGAA |
| | ATGTAATTCTAAAACTTACAACGCGCGATAAATTATTTAATAAAGTATATATAGGTTATTGCATGGCGTG |
| | TTTTATTAATCAATCGTTGGAGGATTTATCGCATACTAGTATTTACAATGTAGAAATTAGAAAGATTAAA |
| | TCATTAAATGATTGTATTAACGACGATAAATACGAATATCTGTCTTATATTTATAATATGCTAGTTAATA |
| | GTAAATGAACTTTTACAGATCTAGTATAATTAGTCAGATTATTAAGTATAATAGACGACTAGCTAAGTCT |
| | ATTATTTGCGAGGATGACTCTCAAATTATTACACTCACGGCATTCGTTAACCAATGCCTATGGTGTCATA |
| | AACGAGTATCCGTGTCCGCTATTTTATTAACTACTGATAACAAAATATTAGTATGTAACAGACGAGATAG |
| | TTTTCTCTATTCTGAAATAATTAGAACTAGAAACATGTTTAGAAAGAAACGATTATTTCTGAATTATTCC |
| | AATTATTTGAACAAACAGGAAAGAAGTATACTATCGTCATTTTTTTCTCTAGATCCAGCTACTGCTGATA |
| | ATGATAGAATAGACGCTATTTATCCGGGTGGCATACCCAAAAGGGGTGAGAATGTTCCAGAGTGTTTATC |
| | CAGGGAAATTAAAGAAGAAGTTAATATAGACAATTCTTTTGTATTCATAGACACTCGGTTTTTTATTCAT |
| | GGCATCATAGAAGATACCATTATTAATAAATTTTTTGAGGTAATCTTCTTTGTCGGAAGAATATCTCTAA |
| | CGAGTGATCAAATCATTGATACATTTAAAAGTAATCATGAAATCAAGGATCTAATATTTTTAGATCCGAA |
| | TTCAGGTAATGGACTCCAATACGAAATTGCAAAATATGCTCTAGATACTGCAAAACTCAAATGTTATGGC |
| | CATAGAGGATGTTATTACGAATCATTAAAAAAATTAACTGAGGATGATTGATTAGAAAATATAAATTAAT |
| | TTACCATCGTGTATTTTTATAACGGGATTGTCCGGCATATCATGTAGATAGTTACCGTCTACATCGTATA |
| | CTCGACCATCTACGCCTTTAAATCCTCTATTTATTGACATTAATCTATTAGAATTGGAATACCAAATATT |
| | AGTACCCTCAATTAGTTTATTGGTAATATTTTTGTTAGACGATAGATCGATGGCTCTTGAAACCAAGGTT |
| | TTCCAACCGGACTCATTGTCGATCGGTGAGAAGTCTTTTTCATTAGCATGAATCCATTCTAATGATGTAT |
| | GTTTAAACACTCTAAACAATTGGACAAATTCTTTTGATTTGCTTTGAATGATTTCAAATAGGTCTTCGTC |
| | TACAGTAGGCATACCATTAGATAATCTAGCCATTATAAAGTGCACGTTTACATATCTACGTTCTGGAGGA |
| | GTAAGAACGTGACTATTGAGACGAATGGCTCTTCCTACTATCTGACGAAGAGACGCCTCGTTCCATGTCA |
| | TATCTAAAATGAAGATATCATTGATTGAGAAGAAGCTAATACCCTCGCCTCCACTAGAAGAGAATACGCA |
| | TGTTTTAATGCATTCTCCGTTAGTGTTTGATTCTTGGTTAAACTCAGCCACCGCCTTGATTCTAGTATCT |
| | TTTGTTCTAGATGAGAACTCTATATTAGAGATACCAAAGACTTTGAAATATAGTAATAAGATTTCTATTC |
| | CTGACTGATTAACAAATGGTTCAAAGACTAGACATTTACCATGGGATGCTAATATTCCCAAACATACATC |
| | TATAAATTTGACGCTTTTCTCTTTTAATTCAGTAAATAGAGAGATATCAGCCGCACTAGCATCCCCTCCC |
| | AATAGTTCTCCCCTTTTAAAGGTATCTAATGCAGATTTAGAAAATTCTCTATCTCTTAATGAATTTTTAA |
| | AATCATTATATAGTGTTGCTATCTCTTGCGCGTATTCGCCCGGATCACGATTTTGTCTTTCAGGAAAGCT |
| | ATCGAACGTAAACGTAGTAGCCATACGTCTCAGAATTCTAAATGATGATATACCTGTTTTTATTTCAGCG |
| | AGTTTAGCCTTTTGATAAATTTCTTCTTGCTTTTTCGACATATTAACGTATCGCATTAATACTGTTTTCT |
| | TAGCGAATGATGCAGACCCTTCTACGTCATCAAAAATAGAAAACTCGTTATTAACTATGTACGAACATAG |
| | GCCTCCTAGTTTGGAGACTAATTCTTTTTCATCAACTAGACGTTTATTCTCAAATAGCGATTGGTGTTGT |
| | AAGGATCCTGGTCGTAGTAAGTTAACCAACATGGTGAATTCTTGCACACTATTGACGATAGGTGTAGCCG |
| | ATAAACAAATCATCTTATGGTTTTTTAATGCGATGGTCTTAGATAAAAAATTATATACTGAACGAGTAGG |
| | ACGGATCTTACCATCTTCTTTGATTAATGATTTAGAAATGAAGTTATGACATTCATCAATAATGACGCAT |
| | ATTCTACTCTTGGAATTAATAGTTTTGATATTAGTAAAAAATTTATTTCTAAAATTTTGATCATCGTAAT |
| | TAATAAAAATACAATCCTTCGTTATCTCTGGAGCGTATCTGAGTATAGTGTTCATCCAAGGATCTTCTAT |
| | CAAAGCCTTTTTCACCAATAAGATAATAGCCCAATTCGTATAAATATCCTTAAGATGTTTGAGAATATAT |
| | ACAGTAGTCATTGTTTTACCGACACCCGTTTCATGGAACAATAAAAGAGAATGCATACTGTCTAATCCTA |
| | AGAAAACTCTTGCTACAAAATGTTGATAATCCTTGAGGCGTACTACGTCCGACCCCATCATTTCAACAGG |
| | CATATTAGTAGTTCTGCGCAATGCATAATCGATATAGGCCGCGTGTGATTTACTCATTTATGAGTGATAA |
| | GTAATAACTATGTTTTAAAAATCACAGCAGTAGTTTAACTAGTCTTCTCTGATGTTTGTTTTCGATACTT |
| | TTTGAATCAGAAGTCATACTAGAATAAAGCAACGAGTGAACGTAATAGAGAGCTTCGTATACTCTATTCG |
| | AAAACTCTAAGAACTTATTAATGAATTCCGTATCCACTGGATTGTTTAAAATACTAAATTGAACACTGTT |
| | CACATCCTTCCAAGAAGAAGACTTAGTGACGGACTTAACATGAGACATAAATAAATCCAAATTTTTTTTA |
| | CAAACATCACTAGCCACCATAATGGCGCTATCTTTTAACCAGCTATCGCTTACGCATTTTAGCAGTCTAA |
| | CATTTTTAAAGAGACTACAATATATTCTCATAGTATCGATTACACCTCTACCGAATAAAGTTGGAAGTTT |
| | AATAATACAATATTTTTCGTTTACAAAATCAAATAATGGTCGAAACACGTCGAAGGTTAACATCTTATAA |
| | TCGCTAATGTATAGATTGTTTTCAGTGAGATGATTATTAGATTTAATAGCATCTCGTTCACGTTTGAACA |
| | GTTTATTGCGTGCGCTGAGGTCGGCAACTACGGCGTCCGCTTTAGTACTCCTCCCATAATACTTTACGCT |
| | ATTAATCTTTAAAATTTCATAGACTTTATCTAGATCGCTTTCTGGTAACATGATATCATGTGTAAAAAGT |
| | TTTAACATGTCGGTCGGCATTCTATTTAGATCATTAACTCTAGAAATCTGAAGAAAGTAATTAGCTCCGT |
| | ATTCCAGACTAGGTAATGGGCTTTTACCTAGAGACAGATTAAGTTCTGGCAATGTTTCATAAAATGGAAG |
| | AAGGACATGCGTTCCCTCCCGGATATTTTTTACAATTTCATCCATTTACAACTCTATAGTTTGTTTTCAT |
| | TATTATTAGTTATTATCTCCCATAATCTTGGTAATACTTACCCCTTGATCGTAAGATACCTTATACAGGT |
| | CATTACATACAACTACCAATTGTTTTTGTACATAATAGATTGGATGGTTGACATCCATGGTGGAATAAAC |
| | TACTCGAACAGATAGTTTATCTTTCCCCCTAGATACATTAGCCGTAATAGTTGTCGGCCTAAAGAATATC |
| | TTTGGTGTAAAGTTAAAAGTTAGGGTTCTTGTTCCATTATTGCTTTTTGTCAGTAGTTCATTATAAATTC |
| | TCGAGATGGGTCCGTTCTCTGAATATAGAACATCATTTCCAAATCTAACTTCTAGTCTAGAAATAATATC |
| | GGTCTTATTCTTAAAATCTATTCCCTTGATGAAGGGATCGTTAATGAACAAATCCTTGGCCTTTGATTCG |
| | GCTGATCTATTATCTCCGTTATAGACGTTACGTTGACTAGTCCAAAGACTTACAGGAATAGATGTATCGA |
| | TGATGTTGATACTATGTGATATGTGAGCAAAGATTGTTCTCTTAGTGGCATCACTATATGTTCCAGTAAT |
| | GGCGGAAAACTTTTTAGAAATGTTATATATAAAAGAATTTTTTCGTGTTCCAAACATTAGCAGATTAGTA |
| | TGAAGATAAACACTCATATTATCAGGAACATTATCAATTTTTACATACACATCAGCATCTTGAATAGAAA |
| | CGATACCATCTTCTGGAACCTCTACGATCTCGGCAGACTCCGGATAACCAGTCGGTGGACCATCGCTAAC |
| | AATAACTAGATCATCCAACAATCTACTCACATATGCATCTATATAATCTTTTTCATCTTGTGAGTACCCT |
| | GGATACGAAATAAATTTATTATCCGTATTTCCATAATAAGGTTTAGTATAAACAGAGAGCGATGTTGCCG |
| | CATGAACTTCAGTTACAGTCGCCGTTGGTTGGTTTATTTGACCTATTACTCTCCTAGGTTTCTCTATAAA |
| | TGATGGTTTAATTTGTACATTCTTAACCATATATCCAATAAAGCTCAATTCAGGAACATAAACAAATTCT |
| | TTGTTGAACGTTTCAAAGTCGAACGAAGAGTCACGAATAACGATATCGGATACTGGATTGAAGGTTACCG |
| | TTACGGTAATTTTTGAATCGGATAGTTTAAGACTGCTGAATGTATCTTCCACATCAAACGGAGTTTTAAT |
| | ATAAACGTATACTGTAGATGGTTCTTTAATAGTGTCATTAGGAGTTAGGCCAATAGAAATATCATTAAGT |
| | TCACTAGAATATCCAGAGTGTTTCAAAGCAATTGTATTATTGATACAATTATTATATAATTCTTCGCCCT |
| | CAATTTCCCAAATAACACCGTTACACGAAGAGATAGATACGTGATTAATACATTTATATCCAACATATGG |
| | TACGTAACCGAATCTTCCCATACCTTTAACTTCTGGAAGTTCCAAACTCAGAACCAAATGATTAAGCGCA |
| | GTAATATACTGATCCCTAATTTCGAAGCTAGCGATAGCCTGATTGTCTGGACCATCGTTTGTCATAACTC |
| | CGGATAGAGAAATATATTGCGGCATATATAAAGTTGGAATTTGACTATCGACTGCGAAGACATTAGACCG |
| | TTTAATAGAGTCATCCCCACCGATCAAAGAATTAATGATAGTATTATTCATTTTCTATTTAAAATGGAAA |
| | AAGCTTACAATAAACTCCGTAGAGAAATATCTATAATTTGTGAGTTTTCCTTAAAGTAACAGCTTCCGTA |
| | AACGCCGTCTTTATCTCTTAGTAAGTTTATTGTATTTATAACCTTTTCCTTATCTTCATAGAATACTAAA |
| | GGCAACAAAGAAATTTTTGGTTCTTCTCTAAGAGCTACGTGAGACTTAACCATAGACGCCAACGAATCCC |
| | TACATATTTTAGAACAGAAATACCCAACTTCACCACCCTTGAATGTCTCAATACTAATAGGTTTAAAAAC |
| | CAAATCTTGATTACAAAACCAACACTTATCAATTACACTATTTGTCTTAATAGACACATCTGCCATAGAT |
| | TTATAATACTTTGGTAGTATACAAGCGAGTGCTTCTTCTTTAGCGGGCTTAAAGACTGCTTTAGGTGCTG |
| | AAATAACCACATCTGGAAGGCTTACTCGCTTAGCCATTTAATTACGGAACTATTTTTTTATACTTCTAAT |
| | GAGCAAGTAGAAAACCTCTCATCTACAAAAACATACTCGTGTCCATAATCCTCTACCATAGTTACACGTT |
| | TTTTAGATCTCATATGTGCTAAAAAGTTTTCCCATACTAATTGGTTACTATTATTTTTCGTATAATTTTT |
| | AACAGTTTGAGGTTTTAGATTTTTAGTTACAGAAGTGATATCGAATATTTTATCCAAAAAGAATGAATAA |
| | TTAATTGTCTTAGAAGGAGTGTTTTCTTGGCAAAAGAATACCAAGTGCTTAAATATTTCTACTACTTCAT |
| | TAATCTTTTCTGTACTCAGATTCAGTTTCTCATCTTTTACTTGATTGATTATTTCAAAGACTAACTTATA |
| | ATCCTTTTTATTTATTCTCTCGTTAGCCTTAAGAAAACTAGATACAAAATTTGCATCTACATCATCCGTG |
| | GATATTTGATTTTTTTCCATGATATCCAAGAGTTCCGAGATAATTTCTCCAGAACATTGATGAGACAATA |
| | ATCTCCGCAATACATTTCTCAAATGAATAAGTTTATTAGACACATGGAAGTTTGACTTTTTTTGTACCTT |
| | TGTACATTTTTGAAATACCGACTCGCAAAAAATACAATATTCATATCCTTGTTCAGATACTATACCGTTG |
| | TGTCTACAACCGCTACATAATCGTAGATTCATGTTAACACTCTACGTATCTCGTCGTCCAATATTTTATA |
| | TAAAAACATTTTATTTCTAGACGTTGCCAGAAAATCCTGTAATATTTTTAGTTTTTTGGGCTGTGAATAA |
| | AGTATCGCCCTAATATTGTTACCGTCTTCCGCCAATATAGTAGTTAAATTATCCGCACATGCAAAAGAAC |
| | ACCGCTTAGGCGGATTCAGTACAATGTTATATTTTTCGTACCAACTCATTTAAATATCATAATCTAAAAT |
| | AGTTCTGTAATATGTCTAGCGCTAATATATTGATCATAATCCTGTGCATAAATTAAGATACAACAATGTC |
| | TCGAAATCATCGACATGGCTTCTTCCATAGTTAGAAGATCGTCGTCAAAGTTAGCAACGTGATTCATCAA |
| | CATTTGCTGTTTTGAGGCAGCAAATACTGAACCGTCGCCATTCAACCATTCATAAAAACCATCGTCTGAA |
| | TCCATTGATAATTTCTTGTACTGGTTTTTGAGAGCTCGCATCAATCTAGCATTTCTAGCTCCCGGATTGA |
| | AAACAGAAAGAGGATCGTACATCCAGGGTCCATTTTCTGTAAATAGAATCGTATAATGTCCCTTCAAGAA |
| | GATATCAGACGATCCACAATCAAAGAATTGGTCTCCGAGTTTGTAACAAACTGCGGACTTTAACCTATAC |
| | ATGATACCGTTTAGCATGATTTCTGGTGATACGTCAATCGGAGTATCATCTATTAGAGATCTAAAGCCGG |
| | TGTAACATTCTCCACCAAACATATTCTTATTCTGACGTCGTTCTACATAAAACATCATTGCTCCATTAAC |
| | GATAACAGGGGAATGAACAGCACTACCCATCACATTAGTTCCCAATGGATCAATGTGTGTAACTCCAGAA |
| | CATCTTCCATATCCTATGTTAGGAGGAGCGAACACCACTCTTCCACTATTGCCATCGAATGCCATAGAAT |
| | AAATATCCTTGGAATTGATAGAAATCGGACTGTCGGATGTTGTGATCATCTTCATAGGATTAACAACTAT |
| | GTATGGTGCCGCCTGAAGTTTCATATCGTAACTGATGCCGTTTATAGGTCTAGCCACAGAAACCAACGTA |
| | GGTCTAAATCCAACTATAGACAAAATAGAAGCCAATATCTGTTCCTCATCTGTCATAACTTGAGAGCATC |
| | CAGTATGAATAATCTTCATTAGATGGGGATCTACCGCATCATCATCGTTACAATAAAAAATTCCCATTCT |
| | AATGTTCATAATTGCTTTTCTAATCATGGTATGCATGTTTGCTCTCTGAATCTCTGTGGAAATTAGATCT |
| | GATACACCTGTAATCACTATCGGATTATCCTCCGTAAGACGATTAACCAACAACATATAATTATAAGACT |
| | TTACTTTTCTAAATTCATAAAGTTGCTGGATTAGGCTATAGGTGTCTCCATGTACATACGCGTTCTCGAG |
| | CGCAGGAAGTTTAATACCGAATAGTGCCATCAGAATAGGATGAATATAGTAATTAGTTTCTGGTTTTCTA |
| | TAAATAAAAGACAAATCTTGTGAACTAGACATATCGGTAAAATGCATGGATTGGAATCGTGTAGTCGACA |
| | GAAGAATATGATGATTAGATGGAGAGTATATTTTATCTAACTCTTTGAGTTGGTCACCGATTCTAGGACT |
| | AGCTCGAGAATGAATAAGTACTAAAGGATGAGTACATTTCACAGAAACACTAGCATTGTTCAATGTGCTC |
| | TTTACATGGGTAAGGAGTTGAAATAGCTCGTTTCTATTTGTTCTGACAATATTTAGTTTATTCATAATGT |
| | TAAGCATATCCTGAATAGTAAAGTTAGATGTGTCATACTTGTTAGTAGTTAGATATTTAGCAATTGCATT |
| | CCCATCATTTCTCAATCTCGTACTCCAATCATGTGTAGATGCTACTTCGTCGATGGAAACCATACAATCC |
| | TTTTTGATAGGCTGTTGAGATTGATTATTTCCTGCACGTTTAGGTTTGGTACGTTGATTTCTAGCCCCTG |
| | CGGATATAAAGTCATCGTCTACAATTTGGGACAATGAATTGCATACACTACAAGACAAAGATTTATCAGA |
| | AGTGTGAATATGATCTTCATCTACCAAAGAAAGAGTTTGATTAGTATAACTAGATTTTAGTCCTGCGTTA |
| | GATGTTAAAAAAACATCGCTATTGACCACGGCTTCCATTATTTATATTCGTAGTTTTTACTCGAAAGCGT |
| | GATTTTAATATCCAATCTTATTACTTTTGGAATCGTTCAAAACCTTTGACTAGTTGTAGAATTTGATCTA |
| | TTGCCCTACGCGTATACTCCCTTGCATCATATACGTTCGTCACCAGATCGTTTGTTTCGGCCTGAAGTTG |
| | GTGCATATCTCTTTCAACATTCGACATGAGATCCTTAAGGGCCATATCGTCTAGATTTTGTTGAGATGCT |
| | GCTCCTGGATTTGGATTTTGTTGTGCTGTTGTACATACTGTACCACCAGTAGGTGTAGGAGTACATACAG |
| | TGGCCACAATAGGAGGTTGAGGAGGTGTAACCGTTGGAGTAGTACAAGAAATATTTCCATCCGATTGTTG |
| | TGTACATGTAGTTGTTGGTAACGTCTGAGAAGGTTGGGTAGATGGCGGTGTCGTCGTCTTTTGATCTTTA |
| | TTAAATTTAGAGATAATATCCTGAACAGCATTGCTCGGCGTCAACGCTGGAAGGAGTGAACTCGCCGGCG |
| | CATCAGTATCTTCAGACAGCCAATCAAAAAGATTAGACATATCAGATGATGTATTAGTTTGTTGTCGTGG |
| | TTTTGGTGTAGGAACAGTACTACTAGGTAGAAGAATAGGAGCCGGTGTAGCTGTTGGAACCGGCTGTGGA |
| | GTTATATGAATAGTTGGTTGTAGCGGTTGGATAGGCTGTCTGCTGGCGACCGTCATATTATCTCTAGCTA |
| | GTTGTTCTCGCAACTGTCTTTGATAATACGACTCTTGAGACTTTAGTCCTATTTCAATCGCTTCATCCTT |
| | TTTCGTATCCGGATCCTTTTCTTCAGAATAATAGATTGACGACTTTGGTGTAGAGGATTCTGCCAGCCCC |
| | TGTGAGAACTTGTTAAAGAAGTCCATTTAAGGCTTTAAAATTGAATTGCGATTATAAGATTAAATGGCAG |
| | ACACAGACGATATTATCGACTATGAATCCGATGATCTCACCGAATACGAGGATGATGAAGAAGAGGAAGA |
| | AGATGGAGAGTCACTAGAAACTAGTGATATAGATCCCAAATCTTCTTATAAGATTGTAGAATCAGCATCC |
| | ACTCATATAGAAGATGCGCATTCCAATCTTAAACATATAGGGAATCATATATCTGCTCTTAAACGACGCT |
| | ATACTAGACGTATAAGTCTATTTGAAATAGCGGGTATAATAGCAGAAAGCTATAACTTGCTTCAACGAGG |
| | AAGATTACCTCTAGTTTCAGAATTTTCTGACGAAACGATGAAGCAAAATATGCTACATGTAATTATACAA |
| | GAGATAGAGGAGGGTTCTTGTCCTATAGTCATCGAAAAGAACGGAGAATTGTTGTCGGTAAACGATTTTG |
| | ACAAAGATGGTCTAAAATTCCATCTAGACTATATTATCAAAATTTGGAAACTTCAAAAACGATATTAGAA |
| | TTTATACGAATATCGTTCTCTAAATGTCACAATCAAGTCTCGCATGTTCAGCAATTTATTGTCGTACTTT |
| | ATATCGTGTTCATTAACGATATCTTGCAAAATAGTAATGATTCTATCTTCCTTCGATAGATATTCTTCAG |
| | AGATTATTGTCTTATATTTTTTCTTGTTATCCGATATGAATTTGATAAGACTTTGAACATTATTGATACC |
| | CGTCTGTTTAATTTTTTCTACAGATATTTTAGTTTTGGCAGATTCTATCGTATCTGTCAATAGACATCCA |
| | ACATCGACATTCGACGTCAATTGTCTATAAATCAACGTATAAATTTTAGAAATAACATTAGCGAATTGTT |
| | GTGCATTGATGTCGTTATTCTGAAACAGTATGATTTTAGGTAGCATTTTCTTAACAAAGAGAACGTATTT |
| | ATTGTTACTCAGTTGAACAGATGATATATCCAGATTACTAACGCATCTGATTCCGTATACCAAACTTTCA |
| | GAAGAAATGGTGTACAATTGTTTGTATTCATTCAATGTCTCTTTTTCAGAAATTAGTTTAGAGTCGAATA |
| | CTGCAATAATTTTCAAGAGATAGTTTTCATCAGATAAGATTTTATTTAGTGTAGATATGATAAAACTATT |
| | GTTTTGTTGGAGAACTTGATACGCCGCGTTCTCTGTAGTCGACGCTCTCAAATGGGAAACAATCTCCATT |
| | ATTTTTTTGGAATCGGATACTATATCTTCGGTATCTTGACGCAGTCTAGTATACATAGAGTTAAGAGAGA |
| | TTAGAGTTTGTACATTAAGCAACATGTCTCTAAATGTGGCTACAAACTTTTCCTTTTTCACATAATCTAG |
| | TTTATTATATACCGATTTCACAACGGCACCAGATTTAAGGAACCAGAATGAAAAACTCTGATAACTACAA |
| | TATTTCATCATAGTTACGATTTTATCATCTTCTATAGTTGGTGTAATAGCGCATACCTTTTTCTCCAAGA |
| | CTGGAACCAACGTCATAAAAATGTTTAAATCAAAATCCATATCAACATCTGATGCGCTAAGACCAGTCTC |
| | GCGTTCAAGATTATCTTTACTAATGGTGACGAACTCATCATATAGAACTCTAAGTTTGTCCATTATTTAT |
| | TTACAGATTTAGTTGTTTAATTTATTTGTGCTCTTCCAGAGTTGGGATAGTATTTTTCTAACGTCGGTAT |
| | TATATTATTAGGATCTACGTTCATATGTATCATAATATTAATCATCCACGTTTTGATAAATCTATCTTTA |
| | GCTTCTGAAATAACGTATTTAAACAAAGGAGAAAAATATTTAGCTACGGCATCAGACGCAATAACATTTT |
| | TTGTAAATGTAACGTATTTAGACGACAGATCTTCGTTAAAAAGTTTTCCATCTATGTAGAATCCATCGGT |
| | TGTTAACACCATTCCCGCGTCAGATTGAATAGGAGTTTGAATAGTTTGTTTTGGAAATAGATCCTTCAAT |
| | AACTTATAGTTGGGTGGGAAAAAATCGATTTTATCACTAGACTCTTTCTTTTTTACTATCATTACCTCAT |
| | GAACTATTTCTTGAATGAGTATATGTATTTTCTTTCCTATATCGGACGCGTTCATTGGAAAATATACCAT |
| | GTCGTTAACTATAAGAATATTTTTATCCTCGTTTACAAACTGAATAATATCAGATGTAGTTCGTAAACGA |
| | ACTATATCATCACCAGCACAACATCTAACTATATGATATCCACTAGTTTCCTTTAGCCGTTTATTATCTT |
| | GTTCCATATTAGCAGTCATTCCATCATTTAAGAAGGCGTCAAAGATAATAGGGAGAAATGACATTTTGGA |
| | TTCTGTTACGACTTTACCAAAATTAAGGATATACGGACTTACTATCTTTTTCTCAACGTCGATTTGATGA |
| | ACACACGATGAAAATGTGCTTCTATGAGATTGATCATGTAGAAAACAACAAGGGATACAATATTTCCGCA |
| | TATCATGAAATATATTAAGAAATCCCACCTTATTATATTTCCCCAAAGGATCCATGCATGTAAACATTAT |
| | GCCGTTATCATTAATAAAGACTTCTTTCTCATCGGATTTGTAAAAGTTGTTACTGATTTTTTTCATTCCA |
| | GGATCTAGATAATTAATAATGATGGGTTTTCTATTCTTATTCTTTGTATTTTGGCATATCCTAGACCAGT |
| | AAACAGTTTCCACTTTGGTAAAATCAGCAGACTTTTGAACGCTATTAAACATGGCATTAATGGCAATAAC |
| | TAAAAATGTAAAATATTTTTCTATGTTAGGAATATGGTTTTTCACTTTAATAGATATATGGTTTTTGGCC |
| | AAAATGATAGATATTTTTTTATCCGAGGATAGTAAAATATTATTAGTCGCCGTCTCTATAAAAATGAAGC |
| | TAGTCTCGATATCCAATTTTATTCTAGAATTGATAGGAGTCGCCAAATGTACCTTATACGTTATATCTCC |
| | CTTGATGCGTTCCATTTGTGTATCTATATCGGACACAAGATCTGTAAATAGTTTTACGTTATTAATCATC |
| | ACGGTATCGCCGTCGCTAGATAACGCTAATGTACCATCCAAGTCCCAAATGGAGAGATTTAACTGTTCAT |
| | CGTTTAGAATAAAATGATTACCGGTCATATTAATAAAGTGTTCATCGTATCTAGATAACAACGACTTATA |
| | ATTAATGTCCAAGTCTTGAACTCGCTGAATGATCTTTTTTAACCCAGTTAGTTTTAGATTGGTACGAAAT |
| | ATATTGTTAAACTTTGATTCTACAGTAATGTCCAAATCTAGTTGTGGAAATACTTCCATCAACATTGTTT |
| | CAAACTTGATAATATTATTATCTACATCTTCATACGATCCAAATTCCGGAATAGATGTATCACATGCTCT |
| | TGCCACCCAGATAACCAAAAAGTCACACGCTCCAGGATATACATTGTATAAAAAGCTATCGTTTTTTAGT |
| | AGTGTTTTTTTCTGAGTATATACGAAGGGATTAAAAATAGTATTATCAACGTAACTATATTCCAAATTAT |
| | TCTTATGAGAATAGATAATAATATCGTCCTTAATATCTAACAAATTTCCTAAATATCCCTTTAATTGAGT |
| | CATTCGAAGCGTCAATAGAATATGTCTCTTAACTATTTCCGGCTGTTGTATATTTAAATGACTTCGTAAA |
| | AAATAATATATGGGCGACTTCTCATCTATGTAATCATATGGAGTGAGATATAGGGCTCGTTCTACCTCCT |
| | GCCCCTTACCCACCTGTAATACCAATTGCGGACTTACTATATATCGCATATTTATATCGTGGGGTAAAGT |
| | GAAAATCTACTACCGATGATGTAAGTCTTACAATGTTCGAACCAGTACCAGATCTTAATTTGGAGGCCTC |
| | CGTAGAACTAGGGGAGGTAAATATAGATCAAACAACACCTATGATAAAGGAAAATAGCGGTTTTATATCC |
| | CGTAGTAGACGTCTATTCGCCCATAGATCTAAGGATGATGAGAGAAAACTAGCACTACGATTCTTTTTAC |
| | AAAGACTTTATTTTTTAGATCATAGAGAGATTCATTATTTGTTCAGATGCGTTGACGCTGTAAAAGACGT |
| | CACTATTACCAAAAAAAATAACATTATCGTGGCGCCTTATATAGCACTTTTAACTATCGCATCAAAAGGA |
| | TGCAAACTTACAGAAACAATGATTGAAGCATTTTTTCCAGAACTATATAATGAACATAGTAAGAAATTTA |
| | AATTCAACTCTCAAGTATCCATCATCCAAGAAAAACTCGGATACCAGTTTGGAAACTATCACGTTTATGA |
| | TTTTGAACCGTATTACTCTACAGTAGCTCTGGCTATTCGAGATGAACATTCATCTGGCATTTTTAATATC |
| | CGTCAAGAGAGTTATCTGGTAAGTTCATTATCTGAAATAACATATAGATTTTATCTAATTAATCTAAAAT |
| | CTGATCTTGTTCAATGGAGTGCTAGTACGGGCGCTGTAATTAATCAAATGGTAAATACTGTATTGATTAC |
| | AGTGTATGAAAAGTTACAACTGGTCATAGAAAATGATTCACAATTTACATGTTCATTGGCTGTGGAATCA |
| | AAACTTCCAATAAAATTACTTAAAGATAGAAATGAATTATTTACAAAATTCATTAACGAGTTAAAAAAGA |
| | CCAGTTCATTCAAGATAAGCAAACGCGATAAGGATACGCTACTAAAATATTTTACTTAGGACTGGAGTTA |
| | GAATTTATAGACGACTCATTTCGTTTATCATTGTTACTATTATTACTATTACTATCATTATTAGTGTTGG |
| | CATTATTAGTATTCTTCTTGTCATCTTGTTCAGAAATATACAGCAATGCTATACCTAATACTAAATACAT |
| | TATCATGCTCGCAATGGCTCTAACAACAACGAACCAAAATGAATTTGGTCGTAGCTTTTGTTCACAAAAA |
| | TACATAAAGAAATGTCTACATAAATCTATGGCGCCATTGGCTACTTGAAATAGCGCCAGTCCTCCTACAG |
| | ATTTTAATATAGCTGTATAACATGACATTTATTCATCATCAAAAGAGACAGAGTCACCATCTGTCATATT |
| | TAGATTTTTTTTCATGTGTTCAAAGTATCCTCTACTCATTTCATTATAATAGTTTATCATACTTAGAATT |
| | TTAGGACGGATCAATGAGTAAGACTTGACTAGATCGTCAGTAGTAATTTGTGCATCGTCTATTCTGCATC |
| | CGCTTCGTCGAATAATGTATAGCATCGCTTTGAGATTCTCCATAGCTATCAAGTCTTTATACAATGACAT |
| | GGAAATATCTGTGAATACTTTATACTTCTCCAACATCGATGCCTTAACATCATCGCCTACTTTAGCATTG |
| | AAAATACGTTCTATTGTGTAGATGGATGTAGCAAGATTTTTAAACAACAATGCCATCTTACACGATGATT |
| | GCCTCAAGTCTCCAATCTTTTGTTTAGAACGATTAGCTACAGAGTTCAACGCTTGGCTGACTAGCATATT |
| | ATTATCTTTAGAAATTGTATTCTTCAATGAGGCGTTTATCATATCTGTGATTTCGTTAGTCATATTACAG |
| | TCTGACTGGGTTGTAATGTTATCCAACATATCACCTATGGATACGGTACACGTACCAGCATTTGTAATAA |
| | TCCTATCTAAGATGTTGTATGGCATTGCGCAGAAAATATCTTCTCCTGTAATATCTCCACTCTCGATAAA |
| | TCTACTCAGATTATTCTTAAATGCCTTATTCTCTGGAGAAAAGATATCAGTGTCCATCATTTCATTAATA |
| | GTATACGCAGAAAAGATACCACGAGTATCAATTCTATCCAAGATACTTATCGGTTCCGAGTCACAGATAA |
| | TGGTTTCCTCTCCTTCGGGAGATCCTGCATAGAAATATCTAGGACAATAGTTTCTATACTGTCTGTAACT |
| | CTGATAATCTCTAAAGTCACTAACTGATACCATGAAATTGAGAAGATCAAACGCTGAAGTAATCAATTTT |
| | TCTGCCTCGTTTTTACTACAACTAGTTTTCATCAATGTAGCGACGATGTATTGTTTAGTTACTCTTGGTC |
| | TAATACTGATGATAGAGATATTATTGCTTCCCATAATGGATCTTCTAGTAGTCACCTTAAAGCCCATTGA |
| | TGCGAATAGCAGATAGATAAAGTCTTGGTATGACTCCTTTCTAATATAGTACGGACTACCTTTGTCACCC |
| | AACTTTATACCCACATAAGCCATAACAACCTCTTTAATAGCCGTTTCATGAGGTTTATCAGCCATGAGCC |
| | TGAGTAGTTGGAAGAATCTCATGAATCCCGTCTCAGAAAGTCCTATATGCATGATAGATTTATCTTTCCT |
| | GGGAAACTCTCGTATAGTCATAGATGAAATACTCTTCAAAGTTTCTGAAATAAGATTAGTAACAGTCTTA |
| | CCTCCGACTACTCTGGGTAACAAACATACTCTAATAGGTGTTTTCTCTGCGGAGATAATATCAGAAAGGA |
| | TAGAGCAATAAGTAGTATTATTGTGATTATAAAGACCGAATACATAACAGGTAGAATTTATAAACATCAT |
| | GTCCTGAAGGTTTTTAGACTTGTATTCCTCGTAATCCATACCGTCCCAAAACATGGATTTGGTAACTTTG |
| | ATAGCCGTAGATCTTTGTTCCTTCGCCAACAGGTTAAAGAAATTAATAAAGAATTTGTTGTTTCTATTTA |
| | TGTCCACAAATTGCACGTTTGGAAGCGCCACGGTTACATTCACTGCAGCATTTTGAGGATCGCGAGTATG |
| | AAGTACGATGTTATTGTTTACTGGTATATCTGGAAAGAAATCTACCAGTCTAGGAATAAGAGATTGATAT |
| | CGCATAGAAATAGTAAAGTTTATAATCTCATCATCGAAGAGCATTTTGTTACCATTGTAATAAATATCCA |
| | CTCTGTCATATGTATAAATGAAGTACTGTTCAAACATGATGAGATGTTTATATGTTGGCATAGTAGTGAG |
| | ATCGACGTTTGGTAATGGCAATGTATTAAGATTAACTCCATAATGTCTAGCAGCATCTGCGATGTTATAA |
| | GCGTCGTCAAAGCGGGGTCGATCTTGTATTGTTATATATTGTCTAACACCTATAAGATTATCAAAATCTT |
| | GTCTGCTTAATACACCGTTAACAATTTTTGCCTTGAATTCTTTTATTGGTGCATTAATAACATCCTTATA |
| | GAGGATGTTAAACAAATAAGTGTTATCAAAGTTAAGATCTGGATATTTCTTTTCTGCTAGAACATCCATT |
| | GAGTCGGAGCCATCTGGTTTAATATAACCACCGATAAATCTAGCTCTGTATTCTGTATCCGTCAATCTAA |
| | TATTAAGAAGGTGTTGAGTGAAAGGTGGAAGATCGTAAAAGCTGTGAGTATTAATGATAGGATTAGTTTC |
| | CGAACTAATGTTAATTGGGGTATTAATAATATCTATATTTCCAGCGTTAAGTGTAACATTAAACAGTTTT |
| | AATTCACGTGACGTGGTATCAATTAAATAATTAATGCCCAATTTGGATATAGCAGCCTGAAGCTCATCTT |
| | GTTTAGTTACGGATCCTAATGAGTTATTAAGCAATATATCGAACGGATGAACGAAGGTTGTTTTAAGTTG |
| | GTCACATACTTTGTAATCTAGACATAGATGCGGAAGAACGGTAGAAACTATACGAAATAAATATTCAGAG |
| | TCCTCTAATTGATCAAGAGTAACTATTGACTTAATAGGCATCATTTATTTAGTATTAAATGACGACCGTA |
| | CCAGTGACGGATATACAAAACGATTTAATTACAGAGTTTTCAGAAGATAATTATCCATCTAACAAAAATT |
| | ATGAAATAACTCTTCGTCAAATGTCTATTCTAACTCACGTTAACAACGTGGTAGATAGAGAACATAATGC |
| | CGCCGTAGTGTCATCTCCAGAGGAAATATCCTCACAACTTAATGAAGATCTATTTCCAGATGATGATTCA |
| | CCGGCCACTATTATCGAACGAGTACAACCTCATACTACTATTATTGACGATACTCCACCTCCTACTTTTC |
| | GTAGAGAGTTATTGATATCGGAACAACGTCAACAACGAGAAAAAAGATTTAATATTACAGTATCGAAAAA |
| | TGCTGAAGCAATAATGGAATCTAGATCTATGATAACTTCTATGCCAACACAAACACCATCCTTGGGAGTA |
| | GTTTATGATAAAGATAAAAGAATTCAGATGTTGGAGGATGAAGTGGTTAATCTTAGAAATCAACGATCTA |
| | ATACAAAATCATCTGATAATTTAGATAATTTTACCAAAATACTATTTGGTAAGACTCCGTATAAATCAAC |
| | AGAAGTTAATAAGCGTATAGCCATCGTTAATTATGCAAATTTGAACGGGTCTCCCTTATCAGTCGAGGAC |
| | TTGGATGTTTGTTCAGAGGATGAAATAGATAGAATCTATAAAACGATTAAACAATATCACGAAAGTAGAA |
| | AACAAAAAATTATCGTCACTAACGTGATTATTATTGTCATAAACATTATCGAGCAAGCATTGCTAAAACT |
| | CGGATTTGAAGAAATCAAAGGACTGAGTACCGATATCACTTCAGAAATTATCGATGTGGAGATCGGAGAT |
| | GACTGCGATGCTGTAGCATCAAAACTAGGAATCGGTAACAGTCCGGTTCTTAATATTGTATTGTTTATAC |
| | TCAAGATATTCGTTAAACGAATTAAAATTATTTAATTTAATACATTCCCATATCCAGACAACAATCGTCT |
| | GGATTAATCTGTTCCTGTCGTCTCATACCGGACGACATATTAATCTTTTTATTAGTAGGCATCTTTTTAG |
| | ATGGTTTCTTTTTCCCAGCATTAACTGAGTCGATACCTAGAAGATCGTGATTGATCTCTCCGACCATTCC |
| | ACGAACTTCTAATTGGCCGTCTCTAACGGTACCATAAACTATTTTACCAGCATTAGTAACAGCTTGGACA |
| | ATCTGACCATCCATCGCATTGTACGATGTAGTAGTAACTGTTGTTCTACGTCTAGGAGCACCAGAAGTAT |
| | TTTTGGAGCCCTTGGAGGCTGATGTAGAAGAAGACGAGGATTTTGATTTTGGTTTACATGTAATACATTT |
| | TGAACTCTTTGATTTTGTATCACATGCGCCGGCAGTCACATCTGTTTGAGAATTAAGATTATTGTTGCCT |
| | CCTTTGACGGCTGCATCTCCACCGATTTGCGCTAGTAGATTTTTAAGCTGTGGTGTAATCTTATTAACTG |
| | TTTCGATATAATCATCGTAACTGCTTCTAACGGCTAAATTTTTTTTATCCGCCATTTAGAAGCTAAAAAT |
| | ATTTTTATTTATACAGAAGATTTAACTAGATTATACAATGAACTAATATGATCCTTTTCCAGATTATTTA |
| | CAAACTTGGTATTTTTTGGTTCTGGAGGAGGCGAATTTAAATTCGGACTTGGATTCGGATTTTGTGAGTT |
| | CTTGATCTTATTATACATCGAGTATAGGATGGCGACGGTAACTGCTACACAAATACCGATCAACAAAAGA |
| | ATACCAATCATTTATTGACAATAACTTCACTATTGATCAAGTATGCAATATATCATCTTTTCACTAAATA |
| | AGTAGTAATAATGATTCAACAATGTCGAGATATATGGACGATAATAATTTAGTTCATGGAAATATCGCTA |
| | TGATTGGTATGAATGACTCCGCTAACTCTGTGGGGCGCGCAGTGCTTTCCCCACATAGAATAAATTAGCA |
| | TTCCGACTGTGATAATAATACCAAGTATAAACGCCATAATACTCAATACTTTCCATGTACGAGTGGGACT |
| | GGTAGACTTACTAAAGTCAATAAAGGCGAAGATACACGAAAGAATCAAAAGAATGATTCCAGCGATTAGC |
| | ACGCCGGAAAAATAATTTCCAATCATAAGCATCATGTCCATTTAACTAATAAAAATTTTAAATCGCCGAA |
| | TGAACAAAGTGGAATATAAACCATATAAAAACAATAGTTTGTACTGCAAAAATAATATCTATTTTTGTTT |
| | TCGAAGATATGGTAAAATTAAATAGTAGTACACAGCATGTTATAACTAACAGCAGCAACGGCTCGTAATT |
| | ACTTATCATTTACTAGACGAAAAGGTGGTGGGATATTTTCTTGCTCAAATAATACGAATATATCACCCAT |
| | CCATTTTATGCGATGTTTATATACTCTAATCTTTAATAGATCTATAGACGACGGGTTTACCAACAATATA |
| | GATTTTATCGATTCATCTAATTTAAACCCTTCCTTAAACGTGAATGATCTATTATCTGGCATAACGATGA |
| | CCCTACCTGATGAATCGGACAATGTACTGGGCCATGTAGAATAAATTATCAACGAATTATCGTCTACGAA |
| | CATTTATATCATTTGTTTTAATTTTAGGACGCGAATAAATAGATATAAAATAGAAAATAACAGATATTAC |
| | AACCAGTGTTATGGCCGCGCCCAACCAGGTAGGCAGTTTTATTTTATCTTTTACTACAGGTTCTCCTGGA |
| | TGTACGTCACCAACGGCGGACGTAGTTCTAGTACAATTAGACGTAAGTTCCGCTTGGGAATTTTTTAACG |
| | CTAAAGAGTTAACGTTAATCGTGCACCCAACGTATTTACATCTAGTTCTTTGAACATCTTGATTATAATA |
| | TAACCATTTTCTATCTCTAGATTCGTCGGTGCACTCATGTAACCAACATACCCTAGGTCCTAAATATTTA |
| | TCTCCGGAATTAGATTTTGGATAATTCGCGCACCAACAATTTCTATTTCCTTTATGATCGTTACAAAAGA |
| | CGTATAATGCCGTATCCCCAAAAGTAAAATAATCAGGACGAATAATTCTAATAAACTCAGAACAATATCT |
| | CGCATCCATATGTTTGGAGCAAATATCGGAATAAGTAGACATAGCCGGTTTCCGTTTTGCACGTAACCAT |
| | TCTAAACAATTGGGGTTTCCAGGATCGTTTCTACAAAATCCAGTCATGAAATCGTCACAATGTTCTGTCT |
| | TGTAATTATTATTAAATATTTTTGGACAGTGTTTGGTATTTGTCTTAGAACAACATTTTGCCACGCTATC |
| | ACTATCGCCCAGGAGATAATCCTTTTTTATAAAATGACATCGTTGCCCGGATGCTATATAATCAGTAGCG |
| | TGTTTTAAATCCTTAATATATTCAGGAGTTACCTCGTTCTGATAATAGATTAATGATCCAGGACGAAATT |
| | TGAAAGAACTACATGGTTCTCCATGAATTAATACATATTGTTTAGCAAATTCAGGAACTATAAAACTACT |
| | ACAATGATCTATCGACATACCATCTATCAAACAAAACTTGGGTTTAATTTCTCCCGGAGATGTTTCATAA |
| | TAGTACGTATAACTTTCTTCTGCAAACTTAACAGCTCTATTATATTCAGGATAATTAAAACCTAATTCCA |
| | TATATTTGTCTCGTATATCTGCTATTCCTGGTGCTATTTTGATTCTATTAAGAGTAACAGCTGCCCCCAT |
| | TCTTAATAATCGTCAGTATTTAAACTGTTAAATGTTGGTATATCAACATCTACCTTATTTCCCGCAGTAT |
| | AAGGTTTGTTGCAGGTATACTGTTCAGGAATGGTTACATTTATACTTCTTCTATAGTCCTGTCTTTCGAT |
| | GTTCATCACATATGCAAAGAACAGAATAAACAAAATAATGTAAGAAATAATATTAAATATCTGTGAATTC |
| | GTAAATACATTGATTGCCATAATAATTACAGCAGCTACAATACACACAATAGACATTCCCACAGTGTTGC |
| | CATTACCTCCACGATACATTTGAGTTACTAAGCAATAGGTAATAACTAAGCTAGTAAGAGGCAATAGAAA |
| | AGATGAGATAAATATCATCAATATAGAGATTAGAGGAGGGCTATATAGAGCCAAGACGAACAAAATCAAA |
| | CCGAGTAACGTTCTAACATCATTATTTTTGAAGATTCCCAAATAATCATTCATTCCTCCATAATCGTTTT |
| | GCATCATACCTCCATCTTTAGGCATAAACGATTGCTGCTGTTCCTCTGTAAATAAATCTTTATCAAGCAC |
| | TCCAGCACCCGCAGAGAAGTCGTCAAGCATATTGTAATATCTTAAATAACTCATTTATATATTAAAAAAT |
| | GTCACTATTAAAGATGGAGTATAATCTTTATGCCGAACTAAAAAAAATGACTTGTGGTCAACCCCTAAGT |
| | CTTTTTAACGAAGACGGGGATTTCGTAGAAGTTGAACCGGGATCATCCTTTAAGTTTCTGATACCTAAGG |
| | GATTTTACGCCTCTCCTTCCGTAAAGACGAGTCTAGTATTTGAAACATTAACAACGACCGATAATAAAAT |
| | CACTAGTATCAATCCAACAAATGCGCCAAAGTTATATCCTCTTCAACGCAAAGTCGTATCTGAAGTAGTT |
| | TCTAATATGAGGAAAATGATCGAATCAAAACGTCCTCTATACATTACTCTTCACTTGGCGTGTGGATTTG |
| | GTAAGACTATTACCACGTGTTATCTTATGGCTACACACGGTAGAAAAACCGTCATTTGCGTACCCAATAA |
| | AATGTTAATACATCAATGGAAGACACAGGTAGAGGCAGTCGGATTGGAACATAAGATATCCATAGATGGA |
| | GTAAGTAGTCTATTAAAGGAACTAAAGACTCAAAGTCCGGATGTATTAATAGTAGTCAGTAGACATCTGA |
| | CAAACGATGCCTTTTGTAAATATATCAATAAGCATTATGATTTGTTCATCTTGGATGAATCACATACGTA |
| | TAATCTGATGAACAATACAGCAGTTACAAGATTTTTAGCGTATTATCCTCCGATGATGTGTTATTTTTTA |
| | ACTGCTACACCTAGACCAGCTAACCGAATTTATTGTAACAGTATTATTAATATTGCCAAGTTATCCGATC |
| | TAAAAAAAACTATCTATGCGGTAGATAGTTTTTTTGAGCCATATTCCACAGACAATATTAGACATATGGT |
| | AAAACGACTAGATGGACCATCTAATAAATATCATATATATACCGAGAAGTTATTATCTGTAGACGAGCCT |
| | AGAAATCAACTTATTCTTAATACCCTGGTAGAAGAATTCAAGTCAGGAACTATTAATCGCATTTTAGTTA |
| | TTACTAAACTACGTGAACATATGGTATTATTCTACAAACGATTATTAGATCTTTTCGGACCAGAGGTTGT |
| | ATTTATAGGAGACGCCCAAAATAGACGTACTCCAGATATGGTCAAATCAATCAAGGAACTAAATAGATTT |
| | ATATTCGTATCCACCTTATTTTATTCCGGTACTGGTTTAGATATTCCTAGTTTGGATTCGTTGTTCATTT |
| | GCTCGGCAGTAATCAACAATATGCAAATAGAGCAATTACTAGGGAGGGTATGTCGAGAAACAGAACTATT |
| | AGATAGGACGGTATATGTATTTCCTAACACATCCATCAAAGAAATAAAGTACATGATAGGAAATTTCATG |
| | CAACGAATTATTAGTCTGTCTGTAGATAAACTAGGATTTAAACAAGAAAGTTATCGGAAACATCAAGAAT |
| | CCGATCCCACTTCTGTATGTACAACATCCTCCAGAGAAGAACGTGTATTAAATAGAATATTTAACTCGCA |
| | AAATCGTTAAGAAGTTTAAGCGACGATCCGCATGCTGCGCAGGCCAGTGTATTACCCCTCATAGTATTAA |
| | TATAATCCAATGATACTTTTGTGATGTCGGAAATCTTAACCAATTTAGACTGACAGGCAGAACACGTCAT |
| | GCAATCATCATCGTCATCGATAACTGTAGTCTTGGGCTTCTTTTTGCGGCTCTTCATTCCGGAACGCACA |
| | TTGGTGCTATCCATTTAGGTAGTAAAAAATAAGTCAGAATATGCCCTATAACACGATCGTGCAAAACCTG |
| | GTATATCGTCTCTATCTTTATCACAATATAGTGTATCGACATCTTTATTATTATTGACCTCGTTTATCTT |
| | GGAACATGGAATGGGAACATTTTTGTTATCAACGGCCACCTTTGCCTTAATTCCAGATGTTGTAAAATTA |
| | TAACTAAACAGTCTATCATCGACACAAATGAAATTCTTGTTTAGACGTTTGTAGTTTACGTATGCGGCTC |
| | GTTCGCGTCTCATTTTTTCAGATATTGCAGGTACTATAATATTAAAAATAAGAATGAAATAACATAGGAT |
| | TAAAAATAAAGTTATCATGACTTCTAGCGCTGATTTAACTAACTTAAAAGAATTACTTAGTCTGTACAAA |
| | AGTTTGAAATTTTCAGATTCTGCGGCTATAGAAAAGTATAATTCTTTGGTAGAATGGGGAACATCTACTT |
| | ACTGGAAAATAGGCGTGCAAAAGGTAGCTAATGTCGAGACGTCAATATCTGATTATTATGATGAGGTAAA |
| | AAATAAACCGTTTAATATTGATCCGGGCTATTACATTTTCTTACCGGTATATTTTGGGAGCGTCTTTATT |
| | TATTCGAAGGGTAAAAATATGGTAGAACTTGGATCTGGAAACTCTTTTCAAATACCAGATGATATGCGAA |
| | GTGCGTGTAACAAAGTATTAGACAGCGATAACGGAATAGACTTTCTGAGATTTGTTTTGTTAAACAATAG |
| | ATGGATAATGGAAGATGCTATATCAAAATATCAGTCTCCAGTTAATATATTTAAACTAGCTAGTGAGTAC |
| | GGATTAAACATACCCAAATATTTAGAAATTGAAATAGAGGAAGACACATTATTTGACGACGAGTTATACT |
| | CTATTATAGAACGCTCTTTTGATGATAAATTTCCAAAAATATCCATATCGTATATTAAGTTGGGAGAACT |
| | TAGGCGGCAAGTTGTAGACTTTTTCAAATTCTCGTTCATGTATATTGAGTCCATCAAGGTAGATCGTATA |
| | GGAGATAATATTTTTATTCCTAGCGTTATAACAAAATCAGGAAAAAAGATATTAGTAAAAGATGTAGACC |
| | ATTTAATACGATCTAAGGTTAGAGAACATACATTTGTAAAAGTAAAAAAGAAAAACACATTTTCCATTTT |
| | ATACGACTATGATGGGAACGGAACAGAAACTAGAGGAGAAGTAATAAAACGAATTATAGACACTATAGGA |
| | CGAGACTATTATGTTAACGGAAAGTATTTCTCTAAGGTTGGTAGTGCAGGCTTAAAGCAATTGACTAATA |
| | AATTAGATATTAATGAGTGCGCAACTGTCGATGAGTTAGTTGATGAGATTAATAAATCCGGAACTGTAAA |
| | ACGAAAAATAAAAAACCAATCAGCATTTGATTTAAGCAGAGAATGTTTGGGATATCCAGAAGCAGATTTT |
| | ATAACGTTAGTTAATAACATGCGGTTCAAAATAGAAAATTGTAAGGTTGTAAATTTCAATATTGAAAATA |
| | CTAATTGTTTAAATAACCCGAGTATTGAAACTATATATGGAAACTTTAACCAGTTCGTCTCAATCTTTAA |
| | TATCGTCACCGATGTCAAAAAAAGATTATTCGAGTGAAATAATATGCGCCTTTGATATAGGTGCAAAAAA |
| | TCCTGCCAGAACTGTTTTAGAAGTCAAGGATAACTCCGTTAGGGTATTGGATATATCAAAATTAGACTGG |
| | AGTTCTGATTGGGAAAGGCGCATAGCTAAAGATTTGTCACAATATGAATACACTACAGTTCTTCTAGAAC |
| | GTCAGCCTAGAAGGTCGCCGTATGTTAAATTTATCTATTTTATTAAAGGCTTTTTATATCATACATCGGC |
| | TGCCAAAGTTATTTGCGTCTCGCCTGTCATGTCTGGTAATTCATATAGAGATCGAAAAAAGAGATCGGTC |
| | GAAGCATTTCTTGATTGGATGGACACATTCGGATTGCGAGACTCCGTTCCGGATAGACGCAAATTAGACG |
| | ATGTAGCGGATAGTTTCAATTTGGCTATGAGATACGTATTAGATAAATGGAATACTAATTATACACCTTA |
| | TAATAGGTGTAAATCTAGAAATTACATAAAAAAAATGTAATAACGTTAGTAACGCCATTATGGATAATCT |
| | ATTTACCTTTCTACATGAAATAGAAGATAGATATGCCAGAACTATTTTTAACTTTCATCTAATAAGTTGC |
| | GATGAAATAGGAGATATATATGGTCTTATGAAAGAACGCATTTCCTCAGAGGATATGTTTGATAATATAG |
| | TATATAATAAAGATATACATCCTGCCATTAAGAAACTAGTGTATTGCGACATCCAACTTACTAAACACAT |
| | TATTAATCAGAATACGTATCCGGTATTTAACGATTCTTCACAAGTGAAATGTTGTCATTATTTCGACATA |
| | AACTCAGATAATAGCAATATTAGCTCTCGTACAGTAGAGATATTTGAGAGGGAAAAGTCATCTCTTGTAT |
| | CATATATTAAAACTACCAATAAGAAGAGAAAGGTCAATTACGGCGAAATAAAGAAAACTGTTCATGGAGG |
| | CACTAATGCAAATTACTTTTCCGGTAAAAAGTCTGACGAGTATCTGAGTACTACAGTTAGATCCAACATT |
| | AATCAACCTTGGATCAAAACCATTTCTAAGAGAATGAGAGTAGATATCATTAATCACTCTATAGTAACGC |
| | GTGGAAAAAGCTCTATATTACAAACTATAGAAATTATTTTTACTAATAGAACATGTGTGAAAATATTCAA |
| | GGATTCTACTATGCACATTATTCTATCCAAGGACAAGGATGAAAAGGGGTGTATACACATGATTGACAAA |
| | TTATTCTATGTCTATTATAATTTATTTCTGTTGTTCGAAGATATCATCCAAAACGAGTACTTTAAAGAAG |
| | TAGCTAATGTTGTAAACCACGTACTTACGGCTACGGCATTAGATGAGAAATTATTCCTAATTAAGAAAAT |
| | GGCTGAACACGATGTTTATGGAGTTAGCAATTTCAAAATAGGGATGTTTAACCTGACATTTATTAAGTCG |
| | TTGGATCATACCGTTTTCCCCTCTCTGTTAGATGAGGATAGCAAAATAAAGTTTTTTAAGGGGAAAAAGC |
| | TCAATATTGTAGCATTACGATCTCTGGAGGATTGTATAAATTACGTGACTAAATCCGAGAATATGATAGA |
| | AATGATGAAGGAAAGATCGACTATTTTAAATAGCATAGATATAGAAACGGAATCGGTAGATCGTCTAAAA |
| | GAATTGCTTCTAAAATGAAAAAAAACACTGATTCAGAAATGGATCAACGACTAGGGTATAAGTTTTTGGT |
| | GCCTGATCCTAAAGCCGGAGTTTTTTATAGACCGTTACATTTCCAATATGTATCGTATTCTAATTTTATA |
| | TTGCATCGATTGCATGAAATCTTGACCGTCAAGCGGCCACTCTTATCGTTTAAGAATAATACAGAACGAA |
| | TTATGATAGAAATTAGCAATGTTAAAGTGACTCCTCCAGATTACTCACCTATAATCGCGAGTATTAAAGG |
| | TAAGAGTTATGATGCATTAGCCACGTTCACTGTAAATATCTTTAAAGAGGTAATGACCAAAGAGGGTATA |
| | TCCATCACTAAAATAAGTAGTTATGAGGGAAAAGATTCTCATTTGATAAAAATTCCGCTACTAATAGGAT |
| | ACGGGAATAAAAATCCACTTGATACAGCCAAGTATCTTGTTCCTAATGTCATAGGTGGAGTCTTTATCAA |
| | TAAACAATCTGTCGAAAAAGTAGGAATTAATCTAGTAGAAAAGATTACAACATGGCCAAAATTTAGGGTT |
| | GTTAAGCCAAACTCATTCACTTTCTCGTTTTCCTCCGTATCCCCTCCTAATGTATTACCGACAAGATATC |
| | GCCATTACAAGATATCTCTGGATATATCACAATTGGAAGCGTTGAATATATCATCGACAAAGACATTTAT |
| | AACGGTCAATATTGTTTTGCTGTCTCAATATTTATCTAGAGTGAGTCTAGAATTCATTAGACGTAGTTTA |
| | TCATACGATATGCCTCCAGAAGTTGTCTATCTAGTAAACGCGATAATAGATAGTGCTAAACGAATTACTG |
| | AATCTATTACTGACTTTAATATTGATACATACATTAATGACCTGGTGGAAGCTGAACACATTAAACAAAA |
| | ATCTCAGTTAACGATCAACGAGTTCAAATATGAAATGCTGCATAACTTTTTACCTCATATGAACTATACA |
| | CCCGATCAACTAAAGGGATTTTATATGATATCTTTACTAAGAAAGTTTCTCTACTGTATCTACCACACTT |
| | CTAGATATCCAGATAGAGATTCGATGGTTTGTCATCGCATCCTAACGTACGGCAAATATTTTGAGACGTT |
| | GGCACATGATGAATTAGAGAATTACATAGGCAACATCCGAAACGATATCATGAACAATCACAAGAACAGA |
| | GGCACTTACGCGGTAAACATTCATGTACTAACAACTCCCGGACTTAATCACGCGTTTTCTAGCTTATTGA |
| | GTGGAAAGTTCAAAAAGTCAGACGGTAGTTATCGAACACATCCTCACTATTCATGGATGCAGAATATTTC |
| | TATTCCTAGGAGTGTTGGATTTTATCCGGATCAAGTAAAGATTTCAAAGATGTTTTCTGTCAGAAAATAC |
| | CATCCAAGTCAATATCTTTACTTTTGTTCATCGGACGTTCCGGAAAGAGGTCCTCAGGTAGGTTTAGTAT |
| | CTCAATTGTCTGTCTTGAGTTCCATTACAAATATACTAACGTCTGAGTATTTGGATTTGGAAAAGAAAAT |
| | TTGTGAGTATATCAGATCATATTATAAAGATGATATAAGTTACTTTGAAACAGGATTTCCAATCACTATA |
| | GAAAATGCTCTAGTCGCATCTCTTAATCCAAATATGATATGTGATTTTGTAACTGACTTTAGACGTAGAA |
| | AACGGATGGGATTCTTCGGTAACTTGGAGGTAGGTATTACTTTAGTTAGGGATCACATGAATGAAATTCG |
| | CATTAATATTGGAGCGGGAAGATTAGTCAGACCATTCTTGGTTGTGGATAACGGAGAGCTCATGATGGAT |
| | GTGTGTCCGGAGTTAGAAAGCAGATTAGACGACATGACATTCTCTGACATTCAGAAAGAGTTTCCGCATG |
| | TCATCGAAATGGTAGATATAGAACAATTTACTTTTAGTAACGTATGTGAATCGGTTCAAAAATTTAGAAT |
| | GATGTCAAAGGATGAAAGAAAGCAATACGATTTATGTGACTTTCCTGCCGAATTTAGAGATGGATATGTG |
| | GCATCTTCATTAGTGGGAATCAATCACAATTCTGGACCCAGAGCTATTCTTGGATGTGCTCAAGCTAAAC |
| | AAGCTATCTCTTGTCTGAGTTCGGATATACGAAATAAAATAGACAATGGAATTCATTTGATGTATCCAGA |
| | GAGGCCAATCGTGATTAGTAAGGCTTTAGAAACTTCAAAGATTGCGGCTAATTGCTTCGGCCAACATGTT |
| | ACTATAGCATTAATGTCGTACAAAGGTATCAATCAAGAGGATGGAATTATCATCAAAAAACAATTTATTC |
| | AGAGAGGCGGTCTCGATATAGTTACCGCAAAGAAACATCAAGTAGAAATTCCGTTGGAAAACTTTAATAA |
| | CAAAGAAAGAGATAGGTCTAACGCCTATTCAAAATTAGAAAGTAATGGATTAGTTAGACTGAATGCTTTC |
| | TTGGAATCCGGAGACGCTATGGCACGAAATATCTCATCAAGAACTCTTGAAGATGATTTTGCTAGAGATA |
| | ATCAGATTAGCTTCGATGTTTCCGAGAAATATACCGATATGTACAAATCTCGCGTTGAACGAGTACAAGT |
| | AGAACTTACTGACAAAGTTAAGGTACGAGTATTAACCATGAAAGAAAGAAGACCCATTCTAGGAGATAAA |
| | TTTACCACTAGAACGAGTCAAAAGGGAACAGTCGCGTATGTCGCGGATGAAACGGAACTTCCATACGACG |
| | AAAATGGTATCACACCAGATGTCATTATTAATTCTACATCCATCTTCTCTAGAAAAACTATATCTATGTT |
| | GATAGAGGTTATTTTAACAGCCGCATATTCTGCTAAGCCGTACAACAATAAGGGAGAAAACCGACCTGTC |
| | TGTTTTCCTAGTAGTAACGAAACATCCATCGATACATATATGCAATTCGCTAAACAATGTTATGAGCATT |
| | CAAATCCGAAATTGTCCGATGAAGAATTATCGGATAAAATCTTTTGTGAAAAGATTCTCTATGATCCTGA |
| | AACGGATAAGCCTTATGCATCCAAAGTATTTTTTGGACCAATTTATTACTTGCGTCTGAGGCATTTAACT |
| | CAGGACAAGGCAACCGTTAGATGTAGAGGTAAAAAGACGAAGCTCATTAGACAGGCGAATGAGGGACGAA |
| | AACGTGGAGGAGGTATCAAGTTCGGAGAAATGGAGAGAGACTGTTTAATAGCGCATGGCGCAGCCAATAC |
| | TATTACAGAAGTTTTAAAAGACTCAGAAGAGGATTATCAAGATGTGTATGTTTGTGAAAATTGTGGAGAC |
| | ATAGCAGCACAAATCAAGGGTATTAATACATGTCTTAGATGTTCAAAACTTAATCTCTCTCCTCTCTTAA |
| | CAAAAATTGATACCACGCACGTATCTAAAGTATTTCTTACTCAAATGAACGCCAGAGGCGTAAAAGTCAA |
| | ATTAGATTTCGAACGAAGGCCTCCTTCGTTTTATAAACCATTAGATAAAGTTGATCTCAAGCCGTCTTTT |
| | CTGGTGTAATATTCTAGTTTGGTAGTAGATACATATCAATATCATCAAATTCGAGATCCGAATTATAAAA |
| | TGGGCGTGGATTGTTAACTATAGAATCGGACGTCTGATATTCGAAAATCTGTGGAGTTTTAGGTTTTGGT |
| | GGAGGTGTAACTGCTACTTGGGATACTGAAGTCTGATATTCAGAAAGCTGGGGGATGTTCTGGTTCGACA |
| | TCCACCGATGGTGTCACATCACTAATCGGTTCGGTAACGTCTGTGGATGGAGGTGCTACTTCTACAGAAC |
| | CTGTAGCCTCAGTTGTCAACGGAGATACATTTTTAATGCGAGGAAATGTATAATTTGGTAATGGTTTCTC |
| | ATGTGGATCTGAAGAAGAGGTAAGATATCTACTAGAAAGATACCGATCACGTTCTAGTTCTCTTTTGTAG |
| | AACTTAACTTTTTCTTTCTCAGCATCTAGTTGATATTCCAACCTCTTCACGTTACTACGTTCAGATTCCA |
| | ATTCACGTTCGCATGGGTTACCTCCGCAGTTTTTACGAGCGATTTCACGTTCAGCCTTCATGCGTCTCTC |
| | CTTCTCTCTATCGAGTTTATCAGAGCAGTCTTTCTGAAGGCGATCGAACTCCATAAATTTCTCCAACGCT |
| | TTGATTGTTTCCATAGATTTCCGAAGTTCAGCTTTTAGGACTGTGATTCTTTTTCTTTCGAATTCACAGC |
| | TGGATGTGCAACCGTTTCCATTACCGCCATCTCTAAGTTTCTTTTCTAGATCGGCAACATTTCATCCCCA |
| | TGCCTTTTACATTCCTCGAGTCTACTGTCGTCGAAATATCGTTCCAGCTCCTTTTCGACATCAATAACTT |
| | TAGCACGTTGTCTCTCAAGCTCTCTTTTGTAGTTATCTGATTCCCTGGCACGTTTAAGATCTTCATGCAA |
| | TTGAGTCAGCTCTTAACACAATCTCTTGCTTCTTCGTCATAGTACTTACAATCACTATGGGATCCATTGT |
| | TACCACGTCTACACTCGGCGAGCTCGCGTTTAAGAGATTCAATTTCCCGTTTGTATTGGTCCATGTTTCC |
| | ATTGCTACCACCATTAGATTTACAGGCTGCTAGTTGTCGTTCGAGATCAGAAATACGGGTTTTCTTGGAA |
| | TTGATTTCGTCGATGTACTTGGCATCGAAACACTTATTAAGTTCTTTTTCCAATTCTACGATTTTATTTC |
| | TTTCGCGAGTCAATTCCCTCCTGTAGTAACTATCTGTTTTGTCAGATTCACGCTCTCTACGTAGACTTTC |
| | TTGCAAGTTACTAATTTGTTCCCTAGCACGTCCGAGTTTAGTTTTATATGCTGAATAGAGTTCTGATTCA |
| | TCCTTTGAGCAGATCTCTAGCGATCGTTTAAGATTCCTGATTCTAGTCTTTAGCCTATTTACCTCCTCAG |
| | AAGATGTTCCGTTACCGTTGCGTTTACACTCGTTAAGCTGTCTATCAAGATCCATGATTCTATCTCTAAG |
| | ACGTTGCATCTCTCTTTCCATATCAGCATTGCTTTCATTATTACGTCTGCAGTCACTCAACTGTCTTTCA |
| | ATATCTGAGATTCTATCTCTAAGACGTCGCATCTCTCTCTGTTTCGGCATTGGTTTCATTATTACGTCTA |
| | CAGTCGTTCAACTGTCTTTCAAGATCTGATATTCTAGATTGGAGTCTGCTAATCTCTGTAGCATTTTCAC |
| | GGCATTCACTCAGTTGTCTTTCAAGATCTGAGATTTTAGATTGGAGTCTGCTAATCTCTGTAAGATTTCC |
| | TCCTCCGCTCTCGATGCAGTCGGTCAACTTATTCTCTAGTTCTCTAATACGCGAACGCAGTGCATCAACT |
| | TCTTGCGTGTCTTCCTGGTTGCGTGTACATTCATCGAGTCTAGATTCGAGATCTCTAACGCGTCGTCGTT |
| | CTTCCTCAAGTTCTCTGCGTACTACAGAAAGCGTGTCCCTATCTTGTTGATATTTAGCAATTTCTGATTC |
| | TAGAGTACTGATTTTGCTTACGTAGTTACTAATAGTTGTCTTGGCCTTATCAAGATCCTCCTTGTATTTG |
| | TCGCATTCCTTGATATCCCTACGAAGTCTGGACAGTTCCCATTCGACATTACGACGTTTATCGATTTCAG |
| | CTCGGAGATCGTCATCGCGTTGTTTTAGCCACATACGACTGAGTTCAAGTTCTCGTTGACAAGATCCATC |
| | TACTTTTCCATTCCTAATAGTATCCAGTTCCTTTTCTAGTTCTGAACGCATTTCTCGTTCCCTATCAAGC |
| | GATTCTCTCAATTCTCGGATAGTCTTCTTATCAATTTCTAATAAATCTGAACCATCATCTGTCCCATTTT |
| | GAATATCCCTGTGTTCTTTGATCTCTTTTGTAAGTCGGTCGATTCTTTCGGTTTTATAAACAGAATCCCT |
| | TTCCAAAGTCCTAATCTTACTGAGTTTATCACTAAGTTCTGCATTCAATTCGGTGAGTTTTCTCTTGGCT |
| | TCTTCCAACTCTGTTTTAAACTCTCCACTATTTCCGCATTCTTCCTCGCATTTATCTAACCATTCAATTA |
| | GTTTATTAATAACTAGTTGGTAATCAGCGATTCCTATAGCCGTTCTTGTAATTGTGGGAACATAATTAGG |
| | ATCTTCTAATGGATTGTATGGCTTGATAGCATCATCTTTATCATTATTAGGGGGATGGACAACCTTAATT |
| | GGTTGGTCCTCATCTCCTCCAGTAGCGTGTGGTTCTTCAATACCAGTGTTAGTAATAGGCTTAGGCAAAT |
| | GCTTGTCGTACGCGGGCACTTCCTCATCCATCAAGTATTTATAATCGGGTTCTACGTCTGAATATTCTTT |
| | TCTAAGAGACGCGACTTCGGGAGTTAGTAGAAGAACTCTGTTTCTGTATCTATCAACGCTGGAATCAATA |
| | CTCAAGTTAAGGATAGCGAATACCTCATCGTCATCATCCGTATCTTCTGAAACACCATCATATGACATTT |
| | CATGAAGTCTAACGTATTGATAAATAGAATCAGATTTAGTATTAAACAGATCCTTAACCTTTTTAGTAAA |
| | CGCATATGTATATTTTAGATCTCCAGATTTCATAATATGATCACATGCCTTAAATGTCAGTGCTTCCATG |
| | ATATAATCTGGAACACTAATGGGTGACGAAAAAGATACAGCACCATATGCTACGTTGATAAATAAATCTG |
| | AACCACTAAGTAGATAATGATTAATGTTAAGGAAGAGGAAATATTCAGTATATAGATATGCCTTAGCATC |
| | ATATCTTGTACTAAACACGCTAAACAGTTTATTGATGTGATCAATTTCCAACAGAATAATTAGAGCAGCG |
| | GGAATACCAACAAACATATTACCACATCCGTATTTTCTATGAATATCACATATCATGTTAAAAAATCTTG |
| | ATAGAAGAGCGAATATCTCGTCTGACTTAATGAGTCGTAGTTCAGCAGCAACATAAGTCATAACTGTAAA |
| | TAGAACATACTTTCCTGTAGTGTTGATTCTAGACTCCACATCAACACCATTATTAAAAATAGTTTTATAT |
| | ACATCTTTAATCTGCTCTCCGTTAATCGTCGAACGTTCTAGTATACGGAAACACTTTGATTTCTTATCTG |
| | TAGTTAATGACTTAGTGATATCACGAAGAATATTACGAATTACATTTCTTGTTTTTCTTGAGAGACCTGA |
| | TTCAGAACTCAACTCATCGTTCCATAGTTTTTTTACCTCAGTGGCGAAATCTTTGGAGTGCTTGGTACAT |
| | TTTTCAATAAGGTTCGTGACCTCCATTTATTATAAAAAATTTTATTCAAAACTTAACTACAATCGGGTAA |
| | TTATAAAATCGTAGATCTCCCATGTGGTGGAATACTACCATCTATCGCATGTGGATGGACAGTAGGTAAT |
| | GGCCATGGGAACAGTAATGTTTGCATATTTATCTTTCTTGCCAGTATTACTGCATATTGTCCCAATGTTT |
| | CGATGTGATGTTCTAACCTATCAACTGCCGCTGTATCACAACAATAGTGTCCGATGAAATTAAGATTATG |
| | ATCCAATGTGTTTAATATATGATTATCAAGTCTTATACGATCCGCGTCTTTTTTGACAGGATCAGGTTCT |
| | TCTACAGGAAGAAGTTTCGGCCTCTTATGATATTCATGTCTGGGAAACGGTGGTCTAGGGTGAGGCTCCG |
| | GTATCGGAGTGGGTTTTGGATTATAATCATCATCGTCTATGACATCATCTTCGACTTCGATATTTATTTT |
| | GCTATCTTGATGATGTCCTGTATCAGTTGCATTTTCAGCACTCGACTGAATATTAGCGCATTCATTGTCT |
| | ATTATTACCATATTTCTAAACCCAAAATGTATGTGTTGAACATCAGTACTATCGTTGATGAGTCTTATAG |
| | CATGAATTCGCTTATCGTTATCGGGTTTATCTTCTGTCACCTTAGCAATTCCTTTTTTATTAAACTCTAC |
| | ATAATCATATCCATTTCTATTGTTTGTTCTAATATAAACGAGTATAGCATCATTGCTAAATTTTTCAATA |
| | GTATCGAAAACAGAATATCCTAAACCATATAATATATATTCAGGGACACTCAAACTAAATGTCCAGGATT |
| | CTCCTAAATACGTAAACTTTAATAGTGCGAAATCATTCAAAAATCTACCACTTATAGATAGATAGTACAT |
| | AAATGCGTATAGTAGTCTACCTATCTCTTTATTATGAAAACCGGCATTACGATCATATATGTCGTGATAT |
| | ACCTGTGATCCGTTTACGTTAAACCATAAATACATGGGTGATCCTATAAACATGAATTTATTTCTAATTC |
| | TCAGAGCTATAGTTAATTGACCGTGTAATATTTGCTTACATGCATACTTGATACGCTCATTAATAAAATT |
| | TTTATCATTGCTCGTTATCTCAGAATCGTATATATAAGGAGTACCATCGTGATTCTTACCAGATATTATA |
| | CAAAATACTATATATAAAATATATTGACCAACGTTAGTAATCATATAAATGTTTAACGTTTTAAATTTTG |
| | TATTCAATGATCCATTATCATACGCTAGCATGGTCTTATGATATTCATTCTTTAAAATATAATATTGTGT |
| | TAGCCATTGCATTGGGGCTCCTAATGGAGATTTTTTATTCTCATCCATTTTAGGATAGGCTTTCATAAAG |
| | TCCCTAATAACTTCGTGAATAATGTTTCTATGTTTTCTACTGATGCATGTATTTGCTTCGATTTTTTTAT |
| | CCCATGTTTCATCTATCATAGATTTAAACGCAGTAATGCTCGCAACATTAACATCTTGAACCGTTGGTAC |
| | AATTCCGTTCCATAAATTTATAATGTTCGCCATTTATATAACTCATTTTTTGAATATACTTTTAATTAAC |
| | AAAAGAGTTAAGTTACTCATATGGGCGCCGTCCAGTCTGAACATCAATCTTTTTAGCCAGAGATATCATA |
| | GCCGCTCTTAGAGTTTCAGCGTGATTTTCCAACCTAAATAGAACTTCATCGTTGCGTTTACAACACTTTT |
| | CTATTTGTTCAAACTTTGTTGTTACATTAGTAATCTTTTTTTCCAAATTAGTTAGCCGTTGTTTGAGAGT |
| | TTCCTCATTGTCGTCTTCATCGGCTTTAACAATTGCTTCGCGTTTAGCCTCTGGCTTTTTAGCAGCCTTT |
| | GTAGAAAAAAATTCAGTTGCTGGAATTGCAAGATCGTCATCTCCGGGGAAAAGAGTTCCGTCCATTTAAA |
| | GTACAGATTTTAGAAACTGACACTCTGCGTTATTTATATTTGGTACAACACATGGATTATAAATATCGAT |
| | GTTAATAACATCAGAAAATGTAAAGTCTATACATTGTTGCATCGTGTTAAATTTTCTAATGGATCTAGTA |
| | TTATTGGGTCCAACTTCTGCCTGAAATCCAAATATGGAAGCGGATACAAAACCGTTTCCTGGATAAACCA |
| | CACATCTCCACTTTTGCTTTACATCAGAAATTGTGTCGTTGACATCTTGAACTCTCCTATCTAATGCCGG |
| | TGTTCCACCTATAGATTTTGAATATTCGAATGCTGCATGAGTAGCATTAAATTCCTTAATATTGCCATAA |
| | TTTTCATATATTGAGTAACCCTGGATAAAAAGTAAACACACCGCAGCCGTCGCTACCACAATAAAAAAAA |
| | TTGATAGAGAGTTCATTTATAATCTATTAGAAGCTGACAAAATTTTTTTACACGCATCAGACAATGCTTT |
| | AATAAATAGTTCAACATCTACTTTTGTCATATCGAACCGATGGTATGATTCTAACCTAGAATTACATCCG |
| | AAAAAGTTGACTATGTTCATAGTCATTAAGTCATTAACAAACAACATTCCAGACTCTGGATTATAAGACG |
| | ATACTGTTTCGTCACAATTACCTACCTTAATCATGTGATTATGAATATTGGCTATTAGAGCACCTTCTAA |
| | GAAATCTATAATATCTTTGAAACACGATTTAAAATCAAACCACGAATATACTTCTACGAAGAAAGTTAGT |
| | TTACCCATAGGAGAAATAACTATAAATGGAGATCTAAATACAAAATCCGGATCTATGATAGTTTTAACAT |
| | TATTATATTCTCTATTAAATACCTCCACATCTAAAAATGTTAATTTTGAAACTATGTCTTCGTTTATTAC |
| | CGTACCTGAACTAAACGCTATAAGCTCTATTGTTTGAGAACTCTTTAAACGATATTCTTGAAATACATGT |
| | AACAAAGTTTCCTTTAACTCGGTCGGTTTATCTACCATAGTTACAGAATTTGTATCCTTATCTATAATAT |
| | AATAATCAAAATCGTATAAAGTTATATAATTATCGCGTTCAGATTGGGATCTTTTCAAATAGACTAAAAA |
| | CCCCATTTCTCTAGTAAGTATCTTATGTATATGTTTGTAAAATATCTTCATGGTGGGAATATGCTCTACC |
| | GCAGTTAGCCATTCCTCATTGACAGCGGTAGATGTATTAGACAAAACTATTCCAATGTTTAACAAGGGCC |
| | ATTTTACGAGATTATTAAATCCTTGTTTGATAAATGTAGCCAATGAGGGTTCGAGTTCAACGACGATTGA |
| | ATTCTCTTCCCGCGGATGCTGCATGATGAACGACGGGATGTTGTTCGATTGATTTGGAATTCTTTTTCGA |
| | CTTTTTGTTTATATTAAATATTTTAAAATTTATAGCGGATAGCAATTCATGTACCACGGATAATGTAGAC |
| | GCGTATTGCGCATCGATATCTTTATTATTAGATAAATTTATCAATAAATGTGAGAAGTTTGCCTCGTTAA |
| | GGTCTTCCATTTAAATATTATATAAACATTTGTGTTTGTATCTTATTCGTCTTTTATGGAATAGTTTTTT |
| | ACTAGTAAAGCTGCAATTACACACTTTGTCCGTAAAACATAAATATAAACACCAGCTTTTATCAATCGTT |
| | CCAAAAAGTCGACGGCGGACATTTTTAACATGGCATCTATTTTAAATACACTTAGGTTTTTGGAAAAAAC |
| | ATCATTTTATAATTGTAACGATTCAATAACTAAAGAAAAGATTAAGATTAAACATAAGGGAATGTCATTT |
| | GTATTTTATAAGCCAAAGCATTCTACCGTTGTTAAATACTTGTCTGGAGGAGGTATATATCATGATGATT |
| | TGGTTGTATTGGGGAAGGTAACAATTAATAATCTAAAGATGATGCTATTTTACATGGATTTATCATATCA |
| | TGGAGTGACAAGTAGTGGAGCAATTTACAAATTGGGATCGTCTATCGATAGACTTTCTCTAAATAGGACT |
| | ATTGTTACAAAAGTTAATAATTATGATGATACATTTTTTGACGACGATGATTGATCGCTATTGCACAATT |
| | TTGTTTTTTTACTTTCTAATATAGCGTTTAGATTCTTTTTCATGTGCGAATATTGATTTACTAAAATATC |
| | GATGTTTAACTTTTGTTCTATGACGTCCTTATCAGCGGTATCGGTACATATACGTAATTCACCTTCACAA |
| | AATACGGAGTCTTCGATAATAATAGCCAATCGATTATTGGATCTAGCTGTCTGTATCATATTCAACATGT |
| | TTAATATATCCTTTCGTTTCCCCTTTACAGGCATCGATCGTAGCATATTTTCCGCGTCTGAGATGGAAAT |
| | GTTAAAACTACAAAAATGCGTAATGTTAGCCCGTCCTAATATTGGTACGTGTCTATAAGTTTGGCATAGT |
| | AGAATAATAGACGTGTTTAAATGCCTTCCAAAGTTTAAGAATTCTATTAGAGTATTGCATTTTGATAGTT |
| | TATCACCTACATCATCAAAAATAAGTAAAAAGTGTGCTGATTTTTTATGATTTTGTGCGACAGCAATACA |
| | TTTTTCTATGTTACTTTTAGTTCGTATCAGATTATATTCTAGAGATTCCTGACTACTAACGAAATTAATA |
| | TGATTTGGCCAAATGTATCCATCATAATCTGGGTTATAAACGGGTGTAAACAAGAATATATGTTTATATT |
| | TTTTAACTAGTGTAGAAAACAGAGATAGTAAATAGATAGTTTTTCCAGATCCAGATCCTCCCGTTAAAAC |
| | CATTCTAAACGGCATTTTTAATAAATTTTCTCTTGAAAATTGTTTTTCTTGGAAACAATTCATAATTATA |
| | TTTACAGTTACTAAATTAATTTGATAATAAATCAAAATATGGAAAACTAAGGTCGTTAGTAGGGAGGAGA |
| | ACAAAGAAGGCACATCGTGACATAAATAACATTTATTATCATGATGACACCAGAAAACGACGAAGAGCAG |
| | ACATCTGTGTTCTCCGCTACTGTTTACGGAGACAAAATTCAAGGAAAGAATAAACGCAAACGCGTGATTG |
| | GTCTATGTATTAGAATATCTATGGTTATTTCACTACTATCTATGATTACCATGTCCGCGTTTCTCATAGT |
| | GCGCCTAAATCAATGCATGTCTGCTAACGAGGCTGCTATTACTGACGCCGCTGTTGCCGTTGCTGCTGCA |
| | TCATCTACTCATAGAAAGGTTGCGTCTAGCACTACACAATATGATCACAAAGAAAGCTGTAATGGTTTAT |
| | ATTACCAGGGTTCTTGTTATATATTACATTCAGACTACCAGTTATTCTCGGATGCTAAAGCAAATTGCAC |
| | TGCGGAATCATCAACACTACCCAATAAATCCGATGTCTTGATTACCTGGCTCATTGATTATGTTGAGGAT |
| | ACATGGGGATCTGATGGTAATCCAATTACAAAAACTACATCCGATTATCAAGATTCTGATGTATCACAAG |
| | AAGTTAGAAAGTATTTTTGTGTTAAAACAATGAACTAATATTTATTTTTGTACATTAATAAATGAAATCG |
| | CTTAATAGACAAACTGTAAGTAGGTTTAAGAAGTTGTCGGTGCCGGCCGCTATAATGATGATACTCTCAA |
| | CCATTATTAGTGGCATAGGAACATTTCTGCATTACAAAGAAGAACTGATGCCTAGTGCTTGCGCCAATGG |
| | ATGGATACAATACGATAAACATTGTTATTTAGATACTAACATTAAAATGTCTACAGATAATGCGGTTTAT |
| | CAGTGTCGTAAATTACGAGCCAGATTGCCTAGACCGGATACTAGACATCTGAGAGTATTGTTTAGTATTT |
| | TTTATAAAGATTATTGGGTAAGTTTAAAAAAGACCAATGATAAATGGTTAGATATTAATAATGATAAAGA |
| | TATAGATATTAGTAAATTAACAAATTTTAAACAACTAAACAGTACGACGGATGCTGAAGCGTGTTATATA |
| | TACAAGTCTGGAAAACTGGTTAAAACAGTATGTAAAAGTACTCAATCTGTACTATGTGTTAAAAAATTCT |
| | ACAAGTGACAACAAAAAATGAATTAATAATAAGTCGTTAACGTACGCCGCCATGGACGCCGCGTTTGTTA |
| | TTACTCCAATGGGTGTGTTGACTATAACAGATACATTGTATGATGATCTCGATATCTCAATCATGGACTT |
| | TATAGGACCATACATTATAGGTAACATAAAAACTGTCCAAATAGATGTACGGGATATAAAATATTCCGAC |
| | ATGCAAAAATGCTACTTTAGCTATAAGGGTAAAATAGTTCCTCAGGATTCTAATGATTTGGCTAGATTCA |
| | ACATTTATAGCATTTGTGCCGCATACAGATCAAAAAATACCATCATCATAGCATGCGACTATGATATCAT |
| | GTTAGATATAGAAGATAAACATCAGCCATTTTATCTATTCCCATCTATTGATGTTTTTAACGCTACAATC |
| | ATAGAAGCGTATAACCTGTATACAGCTGGAGATTATCATCTAATCATCAATCCTTCAGATAATCTGAAAA |
| | TGAAATTGTTGTTTAATTCTTCATTCTGCATATCAGACGGCAATGGATGGATCATAATTGATGGGAAATG |
| | CAATAGTAATTTTTTATCATAAAAGTTGTAAAGTAAATAATAAAACAATAAATATTGAACTAGTAGTACG |
| | TATATTGAGCAATCAGAAATGATGCTGGTACCTCTTATCACGGTGACCGTAGTTGCGGGAACAATATTAG |
| | TATGTTATATATTATATATTTGTAGGAAAAAGATACGTACTGTCTATAATGACAATAAAATTATCATGAC |
| | AAAATTAAAAAAGATAAAGAGTTCTAATTCCAGCAAATCTAGTAAATCAACTGATAGCGAATCAGACTGG |
| | GAGGATCACTGTAGTGCTATGGAACAAAACAATGACGTAGATAATATTTCTAGGAATGAGATATTGGACG |
| | ATGATAGCTTCGCTGGTAGTTTAATATGGGATAACGAATCCAATGTCATGGCGCCTAGCACAGAACACAT |
| | TTACGATAGTGTTGCTGGAAGCACGCTGCTAATAAATAATGATCGTAATGAACAGACTATTTATCAGAAC |
| | ACTACAGTAGTAATTAATGAGACGGAGACTGTTGAAGTACTTAATGAAGATACCAAACAGAATCCTAACT |
| | ATTCATCCAATCCTTTCGTAAATTATAATAAAACCAGTATTTGTAGCAAGTCAAATCCGTTCATTACAGA |
| | ACTCAACAATAAATTTAGTGAGAATAATCCGTTTAGACGAGCACATAGCGATGATTATCTTAATAAGCAA |
| | GAACAAGATCATGAACACGATGATATAGAATCATCGGTCGTATCATTGGTGTGATTAGTTTCCTTTTTAT |
| | AAAATTGAAGTAATATTTAGTATTATTGCTGCCGTCACGTTGTACAAATGGAGATATTCCCTGTATTCGG |
| | CATTTCTAAAATTAGCAATTTTATTGCTAATAATGACTGTAGATATTATATAGATACAGAACATCAAAAA |
| | ATTATATCTGATGAGATCAATAGACAGATGGATGAAACGGTACTTCTTACCAACATCTTAAGCGTAGAAG |
| | TTGTAAATGACAATGAGATGTACCATCTTATTCCCCATAGACTATCGACTATTATACTCTGTATTAGTTC |
| | TGTCGGAGGATGTGTTATCTCTATAGATAATGACATCAATGACAAAAATATTCTAACATTTCCCATTGAT |
| | CATGCTGTAATCATATCCCCACTGAGTAAATGTGTCGTAGTTAGCAAGGGTCCTACAACCATATTGGTTG |
| | TTAAAGCGGATATACCCAGCAAACGATTGGTAACATCGTTTACAAACGACATACTGTATGTAAACAATCT |
| | GTCACTGATTAATTATTTGCCGTTGTCTGTATTCATTATTAGACGAGTCACCGACTATTTGGATAGACAC |
| | ATATGCGATCAGATATTTGCGAATAATAAGTGGTATTCCATTATAACCATCGACGATAAGCAATATCCTA |
| | TTCCATCAAACTGTATAGGTATGTCCTCTGCCAAGTACATAAATTCTAGCATCGAGCAAGATACTTTAAT |
| | ACATGTTTGTAACCTCGAGCATCCATTCGACTTAGTATACAAAAAAATGCAGTCGTACAATTCTGTACCT |
| | ATCAAGGAACAAATATTGTACGGTAGAATTGATAATATAAATATGAGCATTAGTATTTCTGTGGATTAAT |
| | AGATTTCTAGTATGGGGATCATTAATCATCTCTAATCTCTAAATACCTCATAAAACGAAAAAAAAGCTAT |
| | TATCAAATACTGTACGGAATGGATTCATTCTCTTCTCTTTTTATGAAACTCTGTTGTATATCTACTGATA |
| | AAACTGGAAGCAAAAAATCTGATAAAAAGAATAAGAATAAGATCAAGGATTATATGGAACACGATTATTA |
| | TAAAATAACAATAGTTCCTGGTTCCTCTTCCACGTCTACTAGCTCGTGGTATTATACACATGCCTAGTAA |
| | TAGTCTCTTTGCGTTGACGGAAAGCAGACTAGAAATAACAGGCTAAAATGTTCAGACACCATAATAGTTC |
| | CCAACCCAGATAATAACAGAGTACCATCAACACATTCCTTTAAACTCAATCCCAAACCCAAAACCGTTAA |
| | AATGTATCCGGCCAATTGATAGTAGATAATGAGGTGTACAGCGCATGATAATTTACACAGTAACCAAAAT |
| | GAAAATACTTTAGTAATTATAAGAAATATAGATGGTAACGTCATCATCAACAATCCAATAATATGCCGGA |
| | GAGTAAACATTGACGGATAAAACAAAAATGCTCCGCATAACTCTATCATGGCAATAACACAACCAAATAC |
| | TTGTAAGATTCCTAAATTAGTAGAAAATACAACGGATATCGATGTATAAGTGATCTCGAGAAATAATAAG |
| | AATAAAGTAATGCCCGTAAAGATAAACATCAACATTGTTTGGTAATCATTAAACCAATTAGTATGAAGTT |
| | GAACTAATTTCACAGTAGATTTTATTCCAGTGTTATCCTCGCATGTATACGTACCTGGTAAGATATCTTT |
| | ATATTCTATAATCAATGAGACATCACTATCCGATAACGAATGAAGTCTAGCACTAGTATGCCATTTACTT |
| | AATATGGTCGTCTTGGAAGTTTTATTATAAGTTAAAATATCATGGTTGTCCAATTTCCATCTAATATACT |
| | TTGTCGGATTATCTATAGTACACGGAATAATGATGGTATTATTACATGCTGTATACTCTATAGTCTTTGT |
| | AGATGTTATAATCATAAAAGTACAGAGGTATATCAACGATATTCTAACTCTTGACATTTTTTATTTATTT |
| | AAAATGATACCTTTGTTATTTATTTTATTCTATTTTGCTAACGGTATCGAATGGCATAAGTTTGAAACGA |
| | GTGAAGAAATAATTTCTACTTACTTATTAGACGACGTATTATACACGGGTGTTAATGGGGCGGTATACAC |
| | ATTTTCAAATAATAAACTAAACAAAACTGGTTTAGCTAATACTAATTATATCACAACATCTATAAAAGTA |
| | GAGGATGCGGATAAGGATACATTAGTATGCGGAACCAATAACGGAAATCCCAAATGTTGGAAAATAGACG |
| | GTTCAGACGACCCAAAACATAGAGGTAGAGGATACGCTCCTTATCAAAATAGCAAAGTAACGATAATCAG |
| | TCACAACGGATGTGTACTATCTGACATAAACATATCAAAAGAAGGAATTAAACGATGGAGAAGATTTGAC |
| | GGACCATGTGGTTATGATTTATACACGGCGGATAACGTAATTCCAAAAGATGGTTTACGAGGAGCATTCG |
| | TCGATAAAGATGGTACTTATGACAAAGTTTACATTCTTTTCACTGATACTATCGGCTCAAAGAGAATTGT |
| | CAAAATTCCGTATATAGCACAAATGTGCCTAAACGACGAAGGTGGTCCATCATCATTGTCTAGTCATAGA |
| | TGGTCGACGTTTCTCAAAGTCGAATTAGAATGTGATATCGACGGAAGAAGTTATAGACAAATTATTCATT |
| | CTAGAACTATAAAAACAGATAATGATACGATACTATATGTATTCTTCGATAGTCCGCATTATGTACCTAT |
| | TCTATGAATACCATTAAACAATCTTTTTCTACGTCAAAATTGGAAGGATATACAAAGCAATTGCCGTCTC |
| | CAGCTCCTGGTATATGTCTACCAGCTGGAAAAGTTGTTCCACATACCACGTTTGAAGTCATAGAAAAATA |
| | TAATGTACTAGATGATATTATAAAGCCTTTATCTAACCAACCTATCTTCGAAGGACCGTCTGGTGTTAAA |
| | TGGTTCGATATAAAGGAGAAGGAAAATGAACATCGGGAATATAGAATATACTTCATAAAAGAAAATTCTA |
| | TATATTCGTTCGATACAAAATCTAAACAAACTCGTAGCTCGCAAGTCGATGCGCGACTATTTTCAGTAAT |
| | GGTAACTTCGAAACCGTTATTTATAGCAGATATAGGGATAGGAGTAGGAATGCCACAAATGAAAAAAATA |
| | CTTAAAATGTAATCTTAATCGAGTACACCACACGACAATGAACAAACATAAGACAGATTATGCTGGTTAT |
| | GCTTGCTGCGTAATATGCGGTCTAATTGTTGGAATTATTTTTACAGCGACACTATTAAAAGTTGTAGAAC |
| | GTAAATTAGTTCATACACCATCAATAGATAAAACGATAAAAGATGCATATATTAGAGAAGATTGTCCTAC |
| | TGACTGGATAAGCTATAATAATAAATGTATCCATTTATCTACTGATCGAAAAACCTGGGAGGAAGGACGT |
| | AATGCATGCAAAGCTCTAAATCCAAATTCGGATCTAATTAAGATAGAGACTCCAAACGAGTTAAGTTTTT |
| | TAAGAAGCATTAGACGCGGATATTGGGTAGGAGAATCCGAAATATTAAACCAGACAACCCCATATAATTT |
| | TATAGCTAAGAATGCCACGAAGAATGGAACTAAAAAACGGAAATATATTTGTAGCACAACGAATACTCCC |
| | AAACTGCATTCGTGTTACACTATATAACAATTACACTACATTTTTATCATACCACTACTTCGGTTAGATG |
| | TTTTAGAAAAAAATAAATATCGCCGTACCGTTCTTGTTTTTATAAAAATAACAATTAACAATTATCAAAT |
| | TTTTTCTTTAATATTTTACGTGGTTGACCATTCTTGGTGGTAAAATAATCTCTTAGTGTTGGAATGGAAT |
| | GCTGTTTAATGTTTCCACACTCATCGTATATTTTGACGTATGTAGTCACATCGTTTACGCAATAGTCAGA |
| | CTGTAGTTCTATCATGCTTCCTACATTAGAAGGAGGAACAGTTTTAAAGTCTCTTGGTTTTAATCTATTA |
| | CCGTTAGTTTTCATGAAATCCTTTGTTTTATCCACTTCACATTTTAAATAAATGTCCACTATACATTCTT |
| | CTGTTAATTTTACTAGATCGTCATGGGTCATAGAATTTATAGGTTCCGTAGTCCATGGATCCAAACTAGC |
| | AAACTTCGCGTATACGGTATCGCGATTAGTGTATACACCAACTGTATGAAAATTAAGAAAACAGTTTAAT |
| | AGATCAACAGAAATATTTAATCCTCCGTTTGATACAGATGCACCATATTTATGGATTTTGGATTCACACG |
| | TTGTTTGTCTGAGGGGTTCGTCTAGCGTTGCTTCTACATAAACTTCTATTCCCATATATTCTTTATTGTC |
| | AGAATCGCATACCGATTTATCATCATACACTGTTTGAAAACTAAATGGTATACACATCAAAATAACAAAT |
| | ACTAACGAGTACATTCTGCAATATTGTTATCGTAATTGGAAAAATAGTGTTCGAGTGAGTTGGATTATGT |
| | GAGTATTGGATTGTATATTTTATTTTATATTTTGTAATAAGAATAAAATGCTAATGTCAAGTTTATTCCA |
| | ATAGATGTCTTATTAAAAACATATATAATAAATAACAATGGCTGAATGGCATAAAATTATCGAGGATATC |
| | TCAAAAAATAATAAGTTCGAGGATGCCGCCATCGTTGATTACAAGACTACAAAGAATGTTCTAGCTGCTA |
| | TTCCTAACAGAACATTTGCCAAGATTAATCCGGGTGAAATTATTCCTCTCATCACTAATCGTAATATTCT |
| | AAAACCTCTTATTGGTCAGAAATATTGTATTGTATATACTAACTCTCTAATGGATGAGAACACGTATGCT |
| | ATGGAGTTGCTTACTGGGTACGCCCCTGTATCTCCGATCGTTATAGCGAGAACTCATACCGCACTTATAT |
| | TTTTGATGGGTAAGCCAACAACATCCAGACGTGACGTGTATAGAACGTGTAGAGATCACGCTACCCGTGT |
| | ACGCGCAACTGGTAATTAAAATAAAAAGTAATATTCATATGTAGTGTCAATTTTAAATGATGATGATGAA |
| | ATGGATAATATCCATATTGACGATGTCAATAATGCCGGTATTGGCATACAGCTCATCGATTTTTAGATTT |
| | CATTCAGAGGATGTGGAATTATGTTATGGGCATTTGTATTTTGATAGGATCTATAATGTAGTAAATATAA |
| | AATATAATCCGCATATTCCATATAGATATAATTTTATTAATCGCACGTTAACCGTAGATGAACTAGACGA |
| | TAATGTCTTTTTTACACATGGTTATTTTTTAAAACACAAATATGGTTCACTTAATCCTAGTTTGATTGTC |
| | TCATTATCAGGAAACTTAAAATATAATGATATACAATGCTCAGTAAATGTATCGTGTCTCATTAAAAATT |
| | TGGCAACGAGTACATCTACTATATTAACATCTAAACATAAGACTTATTCTCTACATCGGTCCACGTGTAT |
| | TACTATAATAGGATACGATTCTATTATATGGTATAAAGATATAAATGACAAGTATAATGGCATCTATGAT |
| | TTTACTGCAATATGTATGCTAATAGCGTCTACATTGATAGTGACCATATACGTGTTTAAAAAAATAAAAA |
| | TGAACTCTTAATTATGCTATGCTATTAGAAATGGATAAAATCAAAATTACGGTTGATTCAAAAATTGGTA |
| | ATGTTGTTACCATATCGTATAACTTGGAAAAGATAACTATTGATGTCACACCTAAAAAGAAAAAAGAAAA |
| | GGATGTATTATTAGCGCAATCAGTTGCTGTCGAAGAGGCAAAAGATGTCAAGGTAGAAGAAAAAAATATT |
| | ATCGATATTGAAGATGACGATGATATGGATGTAGAAAGCGCATAATACGATCTATAAAAATAAGTATATA |
| | AATACTTTTTATTTACTGTACTCTTACTGTGTAGTGGTGATACCCTACTCGATTATTTTTTTAAAAAAAT |
| | ACTTATTCTGATTCTTCTAGCCATTTCCGTGTTCGTTCGAATGCCACATCGACGTTAAAGATAGGGGAGT |
| | AGTTGAAATCTAGTTCTGCATTGTTGGTACGCACCTCAAATGTAGTGTTGGATATCTTCAACGTATAGTT |
| | GTTGAGTAGTGATGGTTTTCTAAATAGAATTCTCTTCATATCATTCTTGCACGCGTACATTTTTAGCATC |
| | CATCTTGGAATTCTAGATCCTTGTTCTATTCCCAATGGTTTCATCAATAGAAGATTAAACATATCGTACG |
| | AACACGATGGAGAGTAATCGTAGCAAAAGTAAGCATTTCCTTTAATCTCAGATCCCGGATACTGGATATA |
| | TTTTGCAGCCAACACGTGCATCCATGCAACATTTCCTACATATACCCGGCTATGCACCGCGTCATCATCG |
| | ACTGTACGATACATAATGTTACCGTGTTGCTTACATTGCTCGTAAAAGACTTTCGTCAATTTGTCTCCTT |
| | CTCCGTAAATTCCAGTGGGTCTTAGGCAACAAGTATACAATTTTGCTCCATTCATGATTACGGAATTATT |
| | GGCTTTCATAACCAGTTGCTCGGCCATACGTTTACTTTTTGCGTATACATGTCCTGGTGATATATCATAA |
| | AGGGTATGCTCATGGCCGATGAATGGATCACCGTGTTTATTTGGTCCTATTGCTTCCATGCTACTAGTAT |
| | AGATCAAATACTTGATTCCTAGGTCCACACAAGCTGCCAATATAGTCTGTGTTCCATAATAGTTTACTTT |
| | CATGATTTCATTATCGGTGTATTTTCCAAATACATCCACTAGAGCAGCCGTATGAATAATCAGATTTACC |
| | CCATCTAGCGCTTCTCTCACCTTATCAAAGTCGTTTATATCACATTGTATATAGTTTATAACCTTAACTT |
| | TCGAGGTTATTGGTTGTGGATCTTCTACAATATCTATGACTCTGATTTCTTGAACATCATCTGCACTAAT |
| | TAACAGTTTTACTATATACCTGCCTAGAAATCCGGCACCACCAGTAACCGCGTACACGGCCATTGCTGCC |
| | ACTCATAATATCAGACTACTTATTCTATTTTACTAAATAATGGCTGTTTGTATAATAGACCACGATAATA |
| | TCAGAGGAGTTATTTACTTTGAACCAGTCCATGGAAAAGATAAAGTTTTAGGATCAGTTATTGGATTAAA |
| | ATCCGGAACGTATAGTTTGATAATTCATCGTTACGGAGATATTAGTCAAGGATGTGATTCCATAGGCAGT |
| | CCAGAAATATTTATCGGTAACATCTTTGTAAACAGATATGGTGTAGCATATGTTTATTTAGATACAGATG |
| | TAAATATATCTACAATTATTGGAAAGGCGTTATCTATTTCAAAAAATGATCAGAGATTAGCGTGTGGAGT |
| | TATTGGTATTTCTTACATAAATGAAAAGATAATACATTTTCTTACAATTAACGAGAATGGCGTTTGATAT |
| | ATCAGTTAATGCGTCTAAAACAATAAATGCATTAGTTTACTTTTCTACTCAGCAAAATAAATTAGTCATA |
| | CGTAATGAAGTTAATGATACACACTACACTGTCGAATTTGATAGGGACAAAGTAGTTGACACGTTTATTT |
| | CATATAATAGACATAATGACACCATAGAGATAAGAGGGGTGCTTCCAGAGGAAACTAATATTGGTTGCGC |
| | GGTTAATACGCCGGTTAGTATGACTTACTTGTATAATAAGTATAGTTTTAAACTGATTTTAGCAGAATAT |
| | ATAAGACACAGAAATACTATATCCGGCAATATTTATTCGGCATTGATGACACTAGATGATTTGGCTATTA |
| | AACAGTATGGAGACATTGATCTATTATTTAATGAGAAACTTAAAGTAGACTCCGATTCGGGACTATTTGA |
| | CTTTGTCAACTTTGTAAAGGATATGATATGTTGTGATTCTAGAATAGTAGTAGCTCTATCTAGTCTAGTA |
| | TCTAAACATTGGGAATTGACAAATAAAAAGTATAGGTGTATGGCATTAGCCGAACATATATCTGATAGTA |
| | TTCCAATATCTGAGCTATCTAGACTACGATACAATCTATGTAAGTATCTACGCGGACACACTGAGAGCAT |
| | AGAGGATAAATTTGATTATTTTGAAGACGATGATTCGTCTACATGTTCTGCCGTAACCGACAGGGAAACG |
| | GATGTATAATTTTTTTTATAGCGTGAAGGATATGATAAAAAATATAATTGTTGTATTTATCCCATTCCAA |
| | TCACCTTATATGATTCTGTAACACAATGAAGGAGTCTCATAGATGTATAGAGGTCAGATACTGGTTTGAT |
| | AAACTGTTTATTCCACATGAGTATGTTTGACTTTATGGTTAGACCCGCATACTTTAACAAATCACTGAAA |
| | ATTGGAGTTAGGTATTGACCTCTCAGAATCAGTTGCCGTTCTGGAACATTAAATGTATTTTTTATGATAT |
| | ACTCCAACGCATTTATGTGGGCATACAACAAGTCATTACTAATGGAGTATTCCAAGAGTTTTAGTTGTCT |
| | AGTATTTAACAAGAGAAGAGATTTCAACAGACTGTTTATGAACTCGAATGCCGCCTCATTGTCGCTTATA |
| | TTGATGATGTCGAATTCTCCCAATATCATCACCGATGAGTAGCTCATCTTGTTATCGGGATCCAAGTTTT |
| | CTAAAGATGTCATTAAACCCTCGATCATGAATGGATTTATCATCATCGTTTTTATGTTGGACATGAGCTT |
| | AGTCCGTTTGTCCACATCTATAGACGACGATTTCTGAATTATTTTATATATCCCTCTCTTTAACTCCAGG |
| | AACTTGTCAGGATGGTCTACTTTAATATGTTCTCGTCTAAGAGATGAAAATCTTTGGATGGTTGCACGCG |
| | ACTTTTCTCTAAAGGATGACGTTGCCCAAGATCCTCTCTTAAATGAATCCATCTTATCCTTGGACAAGAT |
| | GGACAGTCTATTTTCCTTAGATGGTTTAATATTTTTGTTACCCATGATCTATAAAGGTAGACCTAATCGT |
| | CTCGGATGACCTATATATTTATTTTCAGTTTTATTATACGCATAAATTGTAAAAAATATGTTAGGTTTAC |
| | AAAAATGTCTCGTGGGGCATTAATCGTTTTTGAAGGATTGGACAAATCTGGAAAAACAACACAATGTATG |
| | AACATCATGGAATCTATACCGGCAAACACGATAAAATATCTTAACTTTCCTCAGAGATCCACTGTCACTG |
| | GAAAGATGATAGATGACTATCTAACTCGTAAAAAAACCTATAATGATCATATAGTTAATCTATTATTTTG |
| | TGCAAATAGATGGGAGTTTGCATCTTTTATACAAGAACAACTAGAACAGGGAATTACTTTAATAGTTGAT |
| | AGATACGCGTTCTCTGGAGTAGCGTATGCCGCCGCTAAAGGCGCGTCAATGACTCTCAGTAAGAGTTATG |
| | AATCTGGATTGCCTAAACCCGACTTAGTTATATTCTTGGAATCTGGTAGCAAAGAAATTAATAGAAACGT |
| | CGGCGAGGAAATTTATGAAGATGTTACATTCCAACAAAAGGTATTACAAGAATATAAAAAAATGATTGAA |
| | GAAGGAGATATTCATTGGCAAATTATTTCTTCTGAATTCGAGGAAGATGTAAAGAAGGAGTTGATTAAGA |
| | ATATAGTTATAGAGGCTATACACACGGTTACTGGACCAGTGGGGCAACTGTGGATGTAATAGTGAAATTA |
| | CATTTTTTATAAATAGATGTTAGTACAGTGTTATAAATGGATGAAGCATATTACTCTGGCAACTTGGAAT |
| | CAGTACTCGGATACGTGTCCGATATGCATACCGAACTCGCATCAATATCTCAATTAGTTATTGCCAAGAT |
| | AGAAACTATAGATAATGATATATTAAACAAGGACATTGTAAATTTTATCATGTGTAGATCAAACTTGGAT |
| | AATCCATTTATCTCTTTCCTAGATACTGTATATACTATTATAGATCAAGAGAACTATCAGACCGAATTGA |
| | TTAATTCATTAGACGACAATGAAATTATCGATTGTATAGTTAATAAGTTTATGAGCTTTTATAAGGATAA |
| | CCTAGAAAATATAGTAGATGCTATCATTACTCTAAAATATATAATGAATAATCCAGATTTTAAAACTACG |
| | TATGCCGAAGTACTCGGTTCCAGAATAGCCGATATAGATATTAAACAAGTGATACGTGAGAATATACTAC |
| | AATTGTCTAATGATATCCGCGAACGATATTTGTGAAAAATATTAAAAAAAAATACTTTTTTTATTAAATG |
| | ACGTCGCTTCGCGAATTTAGAAAATTATGCTGTGATATATATCACGCATCAGGATATAAAGAAAAATCTA |
| | AATTAATTAGAGACTTTATAACAGATAGGGATGATAAATATTTGATCATTAAGCTATTGCTTCCCGGATT |
| | AGACGATAGAATTTATAACATGAACGATAAACAAATTATAAAATTATATAGTATAATATTTAAACAATCT |
| | CAGGAAGATATGCTACAAGATTTAGGATACGGATATATAGGAGACACTATTAGGACTTTCTTCAAAGAGA |
| | ACACAGAAATCCGTCCACGAGATAAAAGCATTTTAACTTTAGAAGACGTGGATAGTTTCTTAACTACGTT |
| | ATCATCCGTAACTAAAGAATCGCATCAAATAAAATTATTGACTGATATCGCATCCGTTTGTACATGTAAT |
| | GATTTAAAATGTGTAGTCATGCTTATTGATAAAGATCTAAAAATTAAAGCGGGTCCTCGGTACGTACTTA |
| | ACGCTATTAGTCCTAATGCCTATGATGTGTTTAGAAAATCTAATAACTTGAAAGAGATAATAGAAAATGC |
| | ATCTAAACAAAATCTAGACTCTATATCTATTTCTGTTATGACTCCAATTAATCCCATGTTAGCGGAATCG |
| | TGTGATTCTGTCAATAAGGCGTTTAAAAAATTTCCATCAGGAATGTTTGCGGAAGTCAAATACGATGGTG |
| | AAAGAGTACAAGTTCATAAAAATAATAACGAGTTTGCCTTCTTTAGTAGAAACATGAAACCAGTACTCTC |
| | TCATAAAGTGGATTATCTCAAAGAATACATACCGAAAGCATTTAAAAAAGCTACGTCTATCGTATTGGAT |
| | TCTGAAATTGTTCTTGTAGACGAACATAATGTACCGCTCCCGTTTGGAAGTTTAGGAATACACAAAAAGA |
| | AAGAATATAAAAACTCTAACATGTGTTTGTTCGTGTTTGACTGTTTGTACTTTGATGGATTCGATATGAC |
| | GGACATTCCATTGTACGAACGAAGATCTTTTCTCAAAGATGTTATGGTTGAAATACCCAATAGAATAGTA |
| | TTCTCAGAGTTGACGAATATTAGTAACGAGTCTCAGTTAACTGACGTATTGGATGATGCACTAACGAGAA |
| | AATTAGAAGGATTGGTCTTAAAAGATATTAATGGAGTATACGAACCGGGAAAGAGAAGATGGTTAAAAAT |
| | AAAGCGAGACTATTTGAACGAGGGTTCCATGGCAGATTCTGCCGATTTAGTAGTACTAGGTGCTTACTAT |
| | GGTAAAGGAGCAAAGGGTGGTATCATGGCAGTCTTTCTAATGGGTTGTTACGACGATGAATCCGGTAAAT |
| | GGAAGACGGTTACCAAGTGTTCAGGACACGATGATAATACGTTAAGGGTTTTGCAAGACCAATTAACGAT |
| | GGTTAAAATTAACAAGGATCCCAAAAAAATTCCAGAGTGGTTGGTAGTTAATAAAATCTATATTCCCGAT |
| | TTTGTAGTAGAGGATCCGAAACAATCTCAGATATGGGAAATTTCAGGAGCAGAGTTTACATCTTCCAAGT |
| | CCCATACCGCAAATGGAATATCCATTAGATTTCCTAGATTTACTAGGATTAGAGAAGATAAAACGTGGAA |
| | AGAATCTACTCATCTAAACGATTTAGTAAACTTGACTAAATCTTAATAGTTACATACAAACTGAAAATTA |
| | AAATAACACTATTTAGTTGGTGGTCGCCATGGATGGTGTTATTGTATACTGTCTAAACGCGTTAGTAAAA |
| | CATGGCGAGGAAATAAATCATATAAAAAATGATTTCATGATTAAACCATGTTGTGAAAGAGTTTGTGAAA |
| | AAGTCAAGAACGTTCACATTGGCGGACAATCTAAAAACAATACAGTGATTGCAGATTTGCCATATATGGA |
| | TAATGCGGTATCCGATGTATGCAATTCACTGTATAAAAAGAATGTATCAAGAATATCCAGATTTGCTAAT |
| | TTGATAAAGATAGATGACGATGACAAGACTCCTACTGGTGTATATAATTATTTTAAACCTAAAGATGTTA |
| | TTCCTGTTATCATATCTATAGGAAAGGATAAAGATGTCTGTGAACTATTAATCTCATCAGACATATCGTG |
| | TGCATGCGTGGAGTTAAATTCATATCACGTAGCCATTCTTCCCATGGATGTTTCCTTTTTTACCAAAGGA |
| | AATGCATCATTGATTATTCTCCTGTTTGATTTCTCTATCGATGCAGCACCTCTCTTAAGAAGTGTAACCG |
| | ATAATAATGTTATTATATCTAGACACCAGCGTCTACATGACGAGCTTCCGAGTTCCAATTGGTTCAAGTT |
| | TTACATAAGTATAAAGTCCGACTATTGTTCTATATTATATATGGTTGTTGATGGATCTGTGATGCATGCG |
| | ATAGCTGATAATAGAACTCACGCAATTATTAGCAAAAATATATTAGACAATACTACGATTAACGATGAGT |
| | GTAGATGCTGTTATTTTGAACCACAGATTAGGATTCTTGATAGAGATGAGATGCTCAATGGATCATCGTG |
| | TGATATGAACAGACATTGTATTATGATGAATTTACCTGATGTAGGCAAATTTGGATCTAGTATGTTGGGG |
| | AAATATGAACCTGACATGATTAAGATTGCTCTTTCGGTGGCTGGTAATTTAATAAGAAATCGAGACTACA |
| | TTCCCGGGAGACGAGGATATAGCTACTACGTTTACGGTATAGCCTCTAGATAATTTTTTTAAGCACGAAA |
| | TAAAAAACATAATTTTAAACCAATCTATTTCATACTATTTTGTGTGATCACCATGGACATAAAGATAGAT |
| | ATTAGTATTTCTGGTGATAAATTTACGGTGACTACTAGGAGGGAAAATGAAGAAAGAAAAAAATATCTAC |
| | CTCTCCAAAAAGAAAAAACTACTGATGTTATCAAACCTGATTATCTTGAGTACGATGACTTGTTAGATAG |
| | AGATGAGATGTTTACTATTCTAGAGGAATATTTTATGTACAGAGGTCTATTAGGCCTCAGAATAAAATAT |
| | GGACGACTCTTTAACGAAATTAAAAAATTCGACAATGATGCGGAAGAACAATTCGGTACTATAGAAGAAC |
| | TCAAGCAGAAACTTAGATTAAATTCTGAAGAGGGAGCAGATAACTTTATAGATTATATAAAGGTACAAAA |
| | ACAGGATATCGTCAAACTTACTGTATACGATTGCATATCTATGATAGGATTGTGTGCATGCGTGGTAGAT |
| | GTTTGGAGAAATGAGAAACTGTTTTCTAGATGGAAATATTGTTTACGAGCTATTAAACTGTTTATTAATG |
| | ATCACATGCTTGATAAGATAAAATCTATACTGCAGAATAGACTAGTATATGTGGAAATGTCATAGAAAGT |
| | TAAAAGTTAATGAGAGCAAAAATATATAAGGTTGTATTCCATATTTGTTATTTTTTCTGTAATAGTTAGA |
| | AAAATACATTCGATGGTCTATCTACCAGATTATTATGTGTTATAAGGTACTTTTTCTCATAATAAACTAG |
| | AGTATGAGTAAGATAGTGTTTTTCAAAACATATAAATCTAAAATTGATGGATGAGATATACAGCTATTAA |
| | TTTCGAAAATATATTTTAATCTGATAACTTTAAACATGGATTTTTGATGGTGGTTTAACGTTTTAAAAAA |
| | AGATTTTGTTATTGTAGTATATGATAATATTAAAAGATGGATATAAAGAATTTGCTGACTGCATGTACTA |
| | TTTTTTACATTACTACATTGGCTACGGCAGATATACCTACTCCGCCACCAACGGGTCATGTGACAAGGGA |
| | GAATATCTTGATAAGAGGCATAATCAATGTTGTAATCGGTGTCCACCTGGAGAATTTGCCAAGGTTAGAT |
| | GTAATGGTAACGATAACACAAAATGTGAACGCTGCCCACCTCATACATATACCACAATCCCAATTATTCT |
| | AATGGATGTCATCAATGTAGAAAATGCCCAACCGGATCATTTGATAAGGTAAAGTGTACCGGAACACAGA |
| | ACAGTAAATGTTCGTGTCTTCCTGGTTGGTATTGTGCTACTGATTCTTCACAGACTGAAGATTGTTGAAA |
| | TTGTGTACCAAAAAGGAGATGTCCATGCGGATACTTTGGTGGAATAGATGAACAAGGAAATCCTATTTGT |
| | AAATCGTGTTGTGTTGGTGAATATTGCGACTACCTACGTAATTATAGACTTGATCCATTTCCTCCATGCA |
| | AACTATCTAAATGTAATTAATTATGATTTTGATGATAATGTTACCATACATTATATCGCTACTTGGTTAG |
| | TGTATTATTCAGTATGAAGACCTATTAATAATTACTTATCTTTTGACGATCTTGTTATAATTATAATATA |
| | AAAATACTTATGGCATAGTAACTCATAATTGCTGACGCGATAAATTCGTAATAATCTGTTTTGTTCAAAT |
| | TTTTATAAGGAATCTACAGGCATAAAAATAAAAATATAATTTATAATATACTCTTACAGCGCGCCATCAT |
| | GAATAACAGCAGTGAATTGATTGCTGTTATTAATGGATTTAGAAATAGTGGACGATTTTGTGATATTAGT |
| | ATAGTTATTAATGATGAAAGGATAAACGCTCATAAACTCATCCTATCTGGAGCCTCCGAATATTTTTCCA |
| | TTCTGTTTTCCAATAATTTTATCGATTCTAATGAATACGAAGTTAATCTAAGTCATTTAGATTATCAAAG |
| | CGTTAACGATTTGATCGATTACATTTATGGGATACCTTTGAGCCTAACTAACGATAACGTGAAATATATT |
| | CTTTCAACCGCTGATTTTTTACAAATTGGATCTGCTATTACGGAGTGTGAAAATTACATACTTAAAAATC |
| | TTTGTTCTAAAAACTGTATCGATTTCTACATATACGCTGATAAATATAATAACAAGAAAATAGAATCAGC |
| | GTCGTTTAACACAATATTACAAAATATTTTGAGACTCATCAACGATGAAAACTTTAAATACTTAACAGAG |
| | GAATCAATGATAAAAATTTTAAGCGATGATATGTTAAATATAAAAAATGAGGATTTTGCACCACTAATTC |
| | TCATTAAATGGTTAGAGAGTACTCAACAATCATGCACCGTCGAGTTACTTAGATGCCTCAGAATATCATT |
| | GCTTTCCCCACAAGTTATAAAATCACTTTATAGTCATCAACTGGTTAGTTCAATCTACGAATGTATAACA |
| | TTCTTAAACAATATAGCATTCTTGGATGAATCATTTCCTAGATACCATAGCATCGAGTTGATATCTATCG |
| | GTATAAGTAATTCGCATGATAAGATTTCCATAAACTGCTACAATCATAAAAAAAATACATGGGAAATGAT |
| | ATCTTCACGTAGATATAGGTGTAGTTTCGCAGTGGCCGTCCTGGATAATATTATCTATATGATGGGTGGA |
| | TATGATCAGTCCCCGTATAGAAGTTCAAAGGTTATAGCGTACAATACATGTACAAATTCTTGGATATATG |
| | ATATACCAGAGCTAAAATATCCTCGTTCTAATTGTGGGGGACTGGCTGATGACGAATACATTTATTGTAT |
| | AGGCGGCATACGCGATCAGGATTCATCGTTGACATCTAGTATTGATAAATGGAAGCCATCAAAACCATAT |
| | TGGCAGAAGTATGCTAAAATGCGCGAACCAAAATGTGATATGGGGGTTGCGATGTTAAACGGATTAATAT |
| | ATGTCATGGGTGGAATCGTTAAAGGTGACACGTGTACCGACGCACTAGAGAGTTTATCAGAAGATGGATG |
| | GATGAAGCATCAACGTCTTCCAATAAAAATGTCCAATATGTCGACGATTGTTCATGATGGCAAGATTTAT |
| | ATATCTGGAGGTTACAACAATAGTAGTGTAGTTAATGTAATATCGAATCTAGTCCTTAGCTATAATCCGA |
| | TATATGATGAATGGACCAAATTATCATCATTAAACATTCCTAGAATTAATCCCGCTCTATGGTCAGCGCA |
| | TAATAAATTATATGTAGGAGGAGGAATATCTGATGATGTTCGAACTAATACATCTGAAACATACGATAAA |
| | GAAAAAGATTGTTGGACATTGGATAATGGTCACGTGTTACCACGCAATTATATAATGTATAAATGCGAAC |
| | CGATTAAACATAAATATCCATTGGAAAAAACACAGTACACGAATGATTTTCTAAAGTATTTGGAAAGTTT |
| | TATAGGTAGTTGATAGAACAAAATACATAATTTTGTAAAAATAAATCACTTTTTATACTAATATGACACG |
| | ATTACCAATACTTTTGTTACTAATATCATTAGTATACGCTACACCTTTTCCTCAGACATCTAAAAAAATA |
| | GGTGATGATGCAACTCTATCATGTAATCGAAATAATACAAATGACTACGTTGTTATGAGTGCTTGGTATA |
| | AGGAGCCCAATTCCATTATTCTTTTAGCTGCTAAAAGCGACGTCTTGTATTTTGATAATTATACCAAGGA |
| | TAAAATATCTTACGACTCTCCATACGATGATCTAGTTACAACTATCACAATTAAATCATTGACTGCTAGA |
| | GATGCCGGTACTTATGTATGTGCATTCTTTATGACATCAACTACAAATGACACTGATAAAGTAGATTATG |
| | AAGAATACTCCACAGAGTTGATTGTAAATACAGATAGTGAATCGACTATAGACATAATACTATCTGGATC |
| | TACACATTCACCGGAAACTAGTTCTAAGAAACCTGATTATATAGATAATTCTAATTGCTCGTCGGTATTC |
| | GAAATCGCGACTCCGGAACCAATTACTGATAATGTAGAAGATCATACAGACACCGTCACATACACTAGTG |
| | ATAGCATTAATACAGTAAGTGCATCATCTGGAGAATCCACAACAGACGAGACTCCGGAACCAATTACTGA |
| | TAAAGAAGATCATACAGTTACAGACACTGTCTCATACACTACAGTAAGTACATCATCTGGAATTGTCACT |
| | ACTAAATCAACCACCGATGATGCGGATCTTTATGATACGTACAATGATAATGATACAGTACCACCAACTA |
| | CTGTAGGCGGTAGTACAACCTCTATTAGCAATTATAAAACCAAGGACTTTGTAGAAATATTTGGTATTAC |
| | CGCATTAATTATATTGTCGGCCGTGGCAATTTTCTGTATTACATATTATATATATAATAAACGTTCACGT |
| | AAATACAAAACAGAGAACAAAGTCTAGATTTTTGACTTACATAAATGTCTGGGATAGTAAAATCTATCAT |
| | ATTGAGCGGACCATCTGGTTTAGGAAAGACAGCCATAGCCAAAAGACTATGGGAATATATTTGGATTTGT |
| | GGTGTCCCATACCACTAGATTTCCTCGTCCTATGGAACGAGAAGGTGTTGATTACCATTACGTTAACAGA |
| | GAGGCCATCTGGAAGGGAATAGCCGCCGGAAACTTTCTAGAACATACTGAGTTTTTAGGAAATATTTACG |
| | GAACTTCTAAAACAGCTGTGAATACAGCGGCTATTAATAATCGTATTTGTGTGATGGATCTAAACATCGA |
| | CGGTGTTAGAAGTCTTAAAAATACGTACCTAATGCCTTACTCGGTGTATATAAGACCTACCTCTCTTAAA |
| | ATGGTTGAGACCAAGCTTCGTTGTAGAAACACTGAAGCTAACGATGAGATTCATCGTCGCGTGATATTGG |
| | CAAAAACGGATATGGATGAGGCCAACGAAGCAGGTCTATTCGACACTATTATCATTGAAGATGATGTGAA |
| | TTTAGCATATAGTAAGTTAATTCAGATACTACAGGACCGTATTAGAATGTATTTTAACACTAATTAGAGA |
| | CTTAAGACTTAAAACTTGATAATTAATAATATAACTCGTTTTTATATGTGGCTATTTCAACGTCTAATGT |
| | ATTAGTTAAATATTAAAACTTACCACGTAAAACTTAAAATTTAAAATGATATTTCATTGACAGATAGATC |
| | ACACATTATGAACTTTCAAGGACTTGTGTTAACTGACAATTGCAAAAATCAATGGGTCGTTGGACCATTA |
| | ATAGGAAAAGGTGGATTCGGTAGTATTTATACTACTAATGACAATAATTATGTAGTAAAAATAGAGCCCA |
| | AAGCTAACGGATCATTATTTACCGAACAGGCATTTTATACTAGAGTACTTAAACCATCCGTTATCGAAGA |
| | ATGGAAAAAATCTCACAATATAAAGCACGTAGGTCTTATCACGTGCAAGGCATTTGGTCTATACAAATCC |
| | ATTAATGTGGAATATCGATTCTTGGTAATTAATAGATTAGGTGCAGATCTAGATGCGGTGATCAGAGCCA |
| | ATAATAATAGATTACCAAAAAGGTCGGTGATGTTGATCGGAATCGAAATCTTAAATACCATACAATTTAT |
| | GCACGAGCAAGGATATTCTCACGGAGATATTAAAGCGAGTAATATAGTCTTGGATCAAATAGATAAGAAT |
| | AAATTATATCTAGTGGATTACGGATTGGTTTCTAAATTCATGTCTAATGGAGAACATGTTCCATTTATAA |
| | GAAATCCAAATAAAATGGATAACGGTACTCTAGAATTTACACCTATAGATTCGCATAAAGGATACGTTGT |
| | ATCTAGACGTGGAGATCTAGAAACACTTGGATATTGTATGATTAGATGGTTGGGAGGTATCTTGCCATGG |
| | ACTAAGATATCTGAAACAAAGAATTGTGCATTAGTAAGTGCCACAAAACAGAAATATGTTAACAATACTG |
| | CGACTTTGTTAATGACCAGTTTGCAATATGCACCTAGAGAATTGCTGCAATATATTACCATGGTAAACTC |
| | TTTGACATATTTTGAGGAACCCAATTATGACGAGTTTCGGCACATATTAATGCAGGGTGTATATTATTAA |
| | GTGTGGTGTTTGGTCGATGTAAAATTTTTGTCGATAAAAATTAAAAAATAACTTAATTTATTATTGATCT |
| | CGTGTGTACAACCGAAATCATGGCGATGTTTTACGCACACGCTCTCGGTGGGTACGACGAGAATCTTCAT |
| | GCCTTTCCTGGAATATCATCGACTGTTGCCAATGATGTCAGGAAATATTCTGTTGTGTCAGTTTATAATA |
| | ACAAGTATGACATTGTAAAAGACAAATATATGTGGTGTTACAGTCAGGTGAACAAGAGATATATTGGAGC |
| | ACTGCTGCCTATGTTTGAGTGCAATGAATATCTACAAATTGGAGATCCGATCCATGATCAAGAAGGAAAT |
| | CAAATCTCTATCATCACATATCGCCACAAAAACTACTATGCTCTAAGCGGAATCGGGTACGAGAGTCTAG |
| | ACTTGTGTTTGGAAGGAGTAGGGATTCATCATCACGTACTTGAAACAGGAAACGCTGTATATGGAAAAGT |
| | TCAACATGATTATTCTACTATCAAAGAGAAGGCCAAAGAAATGAATGCACTTAGTCCAGGACCTATCATT |
| | GATTACCACGTCTGGATAGGAGATTGTATCTGTCAAGTTACTGCTGTGGACGTACATGGAAAGGAAATTA |
| | TGAGAATGAGATTCAAAAAGGGTGCGGTGCTTCCGATCCCAAATCTGGTAAAAGTTAAACTTGGGGAGAA |
| | TGATACAGAAAATCTTTCTTCTACTATATCGGCGGCACCATCGAGGTAACCACCTCTCTGGAAGACAGCG |
| | TGAATAATGTACTCATGAAACGTTTGGAAACTATACGCCATATGTGGTCTGTCGTATATGATCATTTTGA |
| | TATTGTGAATGGTAAAGAATGCTGTTATGTGCATACGCATTTGTCTAATCAAAATCTTATACCGAGTACT |
| | GTAAAAACAAATTTGTACATGAAGACTATGGGATCATGCATTCAAATGGATTCCATGGAAGCTCTAGAGT |
| | ATCTTAGCGAACTGAAGGAATCAGGTGGATGGAGTCCCAGACCAGAAATGCAGGAATTTGAATATCCAGA |
| | TGGAGTGGAAGACACTGAATCAATTGAGAGATTGGTAGAGGAGTTCTTCAATAGATCAGAACTTCAGGCT |
| | GGTGAATCAGTCAAATTTGGTAATTCTATTAATGTTAAACATACATCTGTTTCAGCTAAGCAACTAAGAA |
| | CACGTATACGGCAGCAGCTTCCTTTATACTCTCATCTTTTACCAACACAAAGGGTGGATATTTGTTCATT |
| | GGAGTTGATAATAATACACACAAAGTAATTGGATTCACGGTGGGTCATGACTACCTCAGACTGGTAGAGA |
| | ATGATATAGAAAAGCATATCAAAAGACTTCGTGTTGTGCATTTCTGTGAGAAGAAAGAGGACATCAAGTA |
| | CACGTGTCGATTCATCAAGGTATATAAACCTGGGGATGAGGCTACCTCGACATACGTGTGCGCTATCAAA |
| | GTGGAAAGATGCTGTTGTGCTGTGTTTGCAGATTGGCCAGAATCATGGTATATGGATACTAATGGTATCA |
| | AGAAGTATTCTCCAGATGAATGGGTGTCACATATAAAATTTTAATTAATGTAATAGAGAACAAATAATAA |
| | GGTTGTAATATCATATAGACAATAACTAACAATTAATTAGTAACTGTTATCTCTTTTTTAACTAACCAAC |
| | TAACTATATACCTATTAATACATCGTAATTATAGTTCTTAACATCTATTAATCATTAATTCGCTTCTTTA |
| | ATTTTTTATAAACTAACATTGTTAATTGAAAAGGGATAACATGTTACAGAATATAAATTATATATGGATT |
| | TTTTTAAAAAGGAAATACTTGACTGGAGTATATATTTATCTCTTCATTATATAGCACGCGTGTTTTCCAA |
| | TTTTTCCACATCCCATATAATACAGGATTATAATCTCGTTCGAACATACGAGAAAGTGGATAAAACAATA |
| | GTTGATTTTTTATCTAGGTTGCCAAATTTATTCCATATTTTAGAATATGGGGAAAATATTCTACATATTT |
| | ATTCTATGGATGATGCTAATACGAATATTATAATTTTTTTTCTAGATAGAGTATTAAATATTAATAAGAA |
| | CGGGTCATTTATACACAATCTCAGGTTATCATCATCCATTAATATAAAAGAATATGTATATCAATTAGTT |
| | AATAATGATCATCCAGATAATAGGATAAGACTAATGCTTGAAAATGGACGTAGAACAAGACATTTTTTGT |
| | CCTATATATCAGATACAGTTAATATCTATATATGTATTTTAATAAATCATGGATTTTATATAGATGCCGA |
| | AGACAGTTACGGTTGTACATTATTACATAGATGTATATATCACTATAAGAAATCAGAATCAGAATCATAC |
| | AATGAATTAATTAAGATATTGTTAAATAATGGATCAGATGTAGATAAAAAAGATACGTACGGAAACACAC |
| | CTTTTATCCTATTATGTAAACACGATATCAACAACGTGGAATTGTTTGAGATATGTTTAGAGAATGCTAA |
| | TATAGACTCTGTAGACTTTAATAGATATACACCTCTTCATTATGTCTCATGTCGTAATAAATATGATTTT |
| | GTAAAGTTATTAATTTCTAAAGGAGCAAATGTTAATGCGCGTAATAGATTCGGAACTACTCCATTTTATT |
| | GTGGAATTATACACGGTATCTCGCTTATAAAACTATATTTGGAATCAGACACAGAGTTAGAAATAGATAA |
| | TGAACATATAGTTCGTCATTTAATAATTTTTGATGCTGTTGAATCTTTAGATTATCTATTATCCAGAGGA |
| | GTTATTGATATTAACTATCGTACTATATACAACGAAACATCTATTTACGACGCTGTCAGTTATAATGCGT |
| | ATAATACGTTGGTCTATCTATTAAACAGAAATGGTGATTTTGAGACGATTACTACTAGTGGATGTACATG |
| | TATTTCGGAAGCAGTCGCAAACAACAACAAAATAATAATGGAAGTACTATTGTCTAAACGACCATCTTTG |
| | AAAATTATGATACAGTCTATGATAGCAATTACTAAACATAAACAGCATAATGCAGATTTATTGAAAATGT |
| | GTATAAAATATACTGCGTGTATGACCGATTATGATACTCTTATAGATGTACAGTCGCTACAGCAATATAA |
| | ATGGTATATTTTAAAATGTTTCGATGAAATAGATATCATGAAGAGATGTTATATAAAAAATAAAACTGTA |
| | TTCCAATTAGTTTTTTGTATCAAAGACATTAATACTTTAATGAGATACGGTAAACATCCTTCTTTCGTGA |
| | AATGCACTAGTCTCGACGTATACGGAAGTCGTGTACGTAATATCATAGCATCTATTAGATATCGTCAGAG |
| | ATTAATTAGTCTATTATCCAAGAAGCTGGATCCTGGAGATAAATGGTCGTGTTTTCCTAACGAAATAAAA |
| | TATAAAATATTGGAAAACTTTAACGATAACGAACTATCCACATATCTAAAAATCTTATAAACACTATTAA |
| | AATATAAAATCTAAGTAGGATAAAATCACACTACATCATTGTTTCCTTTTAGTGCTCGACAGTGTATACT |
| | ATTTTTAACACTCATAAATAAAAATGAAAACGATTTCCGTTGTTACGTTGTTATGCGTACTACCTGCTGT |
| | TGTTTATTCAACATGTACTGTACCCACTATGAATAACGCTAAATTAACGTCTACCGAAACATCGTTTAAT |
| | GATAAACAGAAAGTTACGTTTACATGTGATCAGGGATATCATTCTTCGGATCCAAATGCTGTCTGCGAAA |
| | CAGATAAATGGAAATACGAAAATCCATGCAAAAAAATGTGCACAGTTTCTGATTACATCTCTGAATTATA |
| | TAATAAACCGCTATACGAAGTGAATTCCACCATGACACTAAGTTGCAACGGCGAAACAAAATATTTTCGT |
| | TGCGAAGAAAAAAATGGAAATACTTCTTGGAATGATACTGTTACGTGTCCTAATGCGGAATGTCAACCTC |
| | TTCAATTAGAACACGGATCGTGTCAACCAGTTAAAGAAAAATACTCATTTGGGGAATATATGACTATCAA |
| | CTGTGATGTTGGATATGAGGTTATTGGTGCTTCGTACATAAGTTGTACAGCTAATTCTTGGAATGTTATT |
| | CCATCATGTCAACAAAAATGTGATATGCCGTCTCTATCTAATGGATTAATTTCCGGATCTACATTTTCTA |
| | TCGGTGGCGTTATACATCTTAGTTGTAAAAGTGGTTTTACACTAACGGGGTCTCCATCATCCACATGTAT |
| | CGACGGTAAATGGAATCCCGTACTCCCAATATGTGTACGAACTAACGAAGAATTTGATCCAGTGGATGAT |
| | GGTCCCGACGATGAGACAGATTTGAGCAAACTCTCGAAAGACGTTGTACAATATGAACAAGAAATAGAAT |
| | CGTTAGAAGCAACTTATCATATAATCATAGTGGCGTTAACAATTATGGGCGTCATATTTTTAATCTCCGT |
| | TATAGTATTAGTTTGTTCCTGTGACAAAAATAATGACCAATATAAGTTCCATAAATTGCTACCGTAAATA |
| | TAAATCCGTTAAAATAATGAATAATTAATAACGAACAAGTATCAAAAGATTAAAGAATTATAGCTAGAAT |
| | CAATTGAGATGTCTTCTTCAGTGGATGTTGATATCTACGATGCCGTTAGAGCATTTTTACTCAGGCACTA |
| | TTATAACAAGAGATTTATTGTGTATGGAAGAAGTAACGCCATATTACATAATATATACAGGCTATTTACA |
| | AGATGCGCCGTTATACCGTTCGATGATATAGTACGTACTATGCCAAATGAATCACGTGTTAAACAATGGG |
| | TGATGGATACACTTAATGGTATAATGATGAATGAACGCGATGTTTCTGTAAGCGTTGGCACCGGAATACT |
| | ATTCATGGAAATGTTTTTCGATTACAATAAAAATAGTATCAACAATCAACTAATGTATGATATAATTAAT |
| | AGCGTATCTATAATTCTAGCTAATGAGAGATATAGAAGCGCTTTTAACGACGATGGTATATACATCCGTA |
| | GAAATATGATTAACAAGTTGTACGGATACGCATCTCTAACTACTATTGGCACGATCGCTGGAGGTGTTTG |
| | TTATTATCTGTTGATGCATCTAGTTAGTTTGTATAAATAATTATTTCAATATACTAGTTAAAATTTTAAG |
| | ATTTTAAATGTATAAAAAACTAATAACGTTTTTATTTGTAATAGGTGCATTAGCATCCTATTCGAATAAT |
| | GAGTACACTCCGTTTAATAAACTGAGTGTAAAACTCTATATAGATGGAGTAGATAATATAGAAAATTCAT |
| | ATACTGATGATAATAATGAATTGGTGTTAAATTTTAAAGAGTACACAATTTCTATTATTACAGAGTCATG |
| | CGACGTCGGATTTGATTCCATAGATATAGATGTTATAAACGACTATAAAATTATTGATATGTATACCATT |
| | GACTCGTCTACTATTCAACGCAGAGGTCACACGTGTAGAATATCTACCAAATTATCATGCCATTATGATA |
| | AGTACCCTTATATTCACAAATATGATGGTGATGAGCGACAATATTCTATTACTGCAGAGGGAAAATGCTA |
| | TAAAGGAATAAAATATGAAATAAGTATGATCAACGATGATACTCTATTGAGAAAACATACTCTTAAAATT |
| | GGATCTACTTATATATTTGATCGTCATGGACATAGTAATACATATTATTCAAAATATGATTTTTAAAAAT |
| | TTAAAATATATTATCACTTCAGTGACAGTAGTCAAATAACAAACAACACCATGAGATATATTATAATTCT |
| | CGCAGTTTTGTTCATTAATAGTATACACGCTAAAATAACTAGTTATAAGTTTGAATCCGTCAATTTTGAT |
| | TCCAAAATTGAATGGACTGGGGATGGTCTATACAATATATCCCTTAAAAATTATGGCATCAAGACGTGGC |
| | AAACAATGTATACAAATGTACCAGAAGGAACATACGACATATCCGCATTTCCAAAGAATGATTTCGTATC |
| | TTTCTGGGTTAAATTTGAACAAGGCGATTATAAAGTGGAAGAGTATTGTACGGGACTATGCGTCGAAGTA |
| | AAAATTGGACCACCGACTGTAACATTGACTGAATACGACGACCATATCAATTTGTACATCGAGCATCCGT |
| | ATGCTACTAGAGGTAGCAAAAAGATTCCTATTTACAAACGCGGTGACATGTGTGATATCTACTTGTTGTA |
| | TACGGCTAACTTCACATTCGGAGATTCTAAAGAACCAGTACCATATGATATCGATGACTACGATTGCACG |
| | TCTACAGGTTGCAGCATAGACTTTGTCACAACAGAAAAAGTGTGCGTGACAGCACAGGGAGCCACAGAAG |
| | GGTTTCTCGAAAAAATTACTCCATGGAGTTCGAAAGTATGTCTGACACCTAAAAAGAGTGTATATACATG |
| | CGCAATTAGATCCAAAGAAGATGTTCCCAATTTCAAGGACAAAATGGCCAGAGTTATCAAGAGAAAATTT |
| | AATAAACAGTCTCAATCTTATTTAACTAAATTTCTCGGTAGCACATCAAATGATGTTACCACTTTTCTTA |
| | GCATGCTTAACTTGACTAAATATTCATAACTAATTTTTATTAATGATACAAAAACGAAATAAAACTGCAT |
| | ATTATACACTGGTTAACGCCCTTATAGGCTCTAACCATTTTCAAGATGAGGTCCCTGATTATAGTCCTTC |
| | TGTTCCCCTCTATCATCTACTCCATGTCTATTAGACAATGTGAGAAGACTGAAGAGGAAACATGGGGATT |
| | GAAAATAGGGTTGTGTATAATTGCCAAAGATTTCTATCCCGAAAGAACTGATTGCAGTGTTCATCTCCCA |
| | ACTGCAAGTGAAGGATTGATAACTGAAGGCAATGGATTCAGGGATATACGAAACACCGATAAATTATAAA |
| | AAAAGCAATGTGTCCGCTGTTTCCGTTAATAATACTATTTTCGTAACTGGCGGATTATTCATAAATAACT |
| | CTAATAGCACGATCGTGGTTAACAATATGGAAAAACTTGACATTTATAAAGACAAACAATGGTCGATTAT |
| | AGAAATGCCTATGGCTAGGGTATATCACGGCATCGACTCGACATTTGGAATGTTATATTTTGCCGGAGGT |
| | CTATCCGTTACCGAACAATATGGTAATTTAGAGAAAAACAACGAGATATCTTGTTACAATCCTAGAACGA |
| | ATAAGTGGTTTGATATTTCATATACTATTTATAAGATATCCATATCATCATTGTGTAAACTAAATAACGT |
| | CTTCTATGTATTTAGTAAGGACATTGGATATGTGGAAAAGTATGATGGTGCATGGAAGTTAGTACATGAT |
| | CGTCTCCCCGCTATAAAGGCATTATCAACTTCTCCTTATTGATTGAAAATGAAAATATAAATAGTTTTTA |
| | TGTATAGCAGTATTACCCTATAGTTTTATTGCTTACTACTAACATGGATACAGATGTTACAAATGTAGAA |
| | GATATCATAAATGAAATAGATAGAGAGAAAGAAGAAATACTAAAAAATGTAGAAATTGAAAATAATAAAA |
| | ACATTAACAAGAATCATCCAAGTGGATATATTAGAGAAGCACTCGTTATTAATACCAGTAGTAATAGTGA |
| | TTCCATTGATAAAGAAGTTATAGAATGTATCTGTCACGATGTAGGAATATAGATCATATCTACTAATTTT |
| | TATAATCGATACAAAACATAAAAAACAACTCGTTATTACATAGCAGGCATGGAATCCTTCAAGTATTGTT |
| | TTGATAACGATGGCAAGAAATGGATTATCGGAAATACTTTATATTCTGGTAATTCAATACTATATAAGGT |
| | CAGAAAAAATTTCACTAGTTCGTTCTACAATTACGTAATGAAGATAGATCACAAATCACACAAGCCATTG |
| | TTGTCTGAAATACGATTCTATATATCTGTATTGGATCCTTTGACTATCGACAACTGGACACGGGAACGTG |
| | GTATAAAGTATTTGGCTATTCCAGATCTGTATGGAATTGGAGAAACCGATGATTATATGTTCTTCGTTAT |
| | AAAGAATTTGGGAAGAGTATTCGCCCCAAAGGATACTGAATCAGTCTTCGAAGCATGCGTCACTATGATA |
| | AACACGTTAGAGTTTATACACTCTCAAGGATTTACCCATGGAAAAATAGAACCGAGGAATATACTGATTA |
| | GAAATAAACGTCTTTCACTAATTGACTATTCTAGAACTAACAAACTATACAAGAGTGGAAACTCACATAT |
| | AGATTACAACGAGGACATGATAACTTCAGGAAATATCAATTATATGTGTGTAGACAATCATCTTGGAGCA |
| | ACAGTTTCAAGACGAGGAGATTTAGAAATGTTGGGATATTGCATGATAGAATGGTTCGGTGGCAAACTTC |
| | CATGGAAAAACGAAAGTAGTATAAAAGTAATAAAACAAAAAAAAGAATATAAAAAATTTATAGCTACTTT |
| | CTTTGAGGACTGTTTTCCTGAAGGAAATGAACCTCTGGAATTAGTTAGATATATAGAATTAGTATACACG |
| | TTAGATTATTCTCAAACTCCTAATTATGACAGACTACGTAAACTGTTTATACAAGATTGAAATTATATTC |
| | TTTTTTTTATAGAGTGTGGTAGTGTTACGGATATTTAATATTAGACTATCTCTATCGCGCTACACGACCA |
| | ATATCGATTACTATGGATATCTTCAGGGAAATCGCATCTTCTATGAAAGGAGAGAATGTATTCATTTCTC |
| | CAGCGTCAATCTCGTCAGTATTGACAATACTGTATTATGGAGCTAATGGATCCACTGCTGAACAGCTATC |
| | AAAATATGTAGAAAAGGAGGAGAACATGGATAAGGTTAGCGCTCAAAATATCTCATTCAAATCCATAAAT |
| | AAAGTATATGGGCGATATTCTGCCGTGTTTAAAGATTCCTTTTTGAGAAAAATTGGCGATAAGTTTCAAA |
| | CTGTTGACTTCACTGATTGTCGCACTATAGATGCAATCAACAAGTGTGTAGATATCTTTACTGAGGGGAA |
| | AATCAATCCACTATTGGATGAACCATTGTCTCCTGATACCTGTCTCCTAGCAATTAGTGCCGTATACTTT |
| | AAAGCAAAATGGTTGACGCCATTCGAAAAGGAATTTACCAGTGATTATCCCTTTTACGTATCTCCGACGG |
| | AAATGGTAGATGTAAGTATGATGTCTATGTACGGCAAGGCATTTAATCACGCATCTGTAAAGGAATCATT |
| | CGGCAACTTTTCAATCATAGAACTGCCATATGTTGGAGATACTAGTATGATGGTCATTCTTCCAGACAAG |
| | ATTGATGGATTAGAATCCATAGAACAAAATCTAACAGATACAAATTTTAAGAAATGGTGTAACTCTCTGG |
| | AAGCTACGTTTATCGATGTTCACATTCCCAAGTTTAAGGTAACAGGCTCGTATAATCTGGTGGATACTCT |
| | AGTAAAGTCAGGACTGACAGAGGTGTTCGGTTCAACTGGAGATTATAGCAATATGTGTAATTCAGATGTG |
| | AGTGTCGACGCTATGATCCACAAAACGTATATAGATGTCAATGAAGAGTATACAGAAGCAGCTGCAGCAA |
| | CTTGTGCACTGGTGTCAGACTGTGCATCAACAATTACAAATGAGTTCTGTGTAGATCATCCGTTCATCTA |
| | TGTGATTAGGCATGTTGATGGAAAAATTCTTTTCGTTGGTAGATATTGCTCTCCGACAACTAATTGTTAA |
| | CCATTTTTTTTAAAAAATAGAAAAAACATGTGGTATTAGTGCAGGTCGTTATTCTTCCAATTGCAATTGG |
| | TAAGATGACGGCCAACTTTAGTACCCACGTCTTTTCACCACAGCACTGTGGATGTGACAGACTGACCAGT |
| | ATTGATGACGTCAGACAATGTTTGACTGAATATATTTATTGGTCGTCCTATGCATACCGCAACAGGCAAT |
| | GCGCTGGACAATTGTATTCCACACTCCTCTCTTTTAGAGATGATGCGGAATTAGTGTTCATCGACATTCG |
| | CGAGCTGGTAAAAAATATGCCGTGGGATGATGTCAAAGATTGTGCAGAAATCATCCGTTGTTATATACCG |
| | GATGAGCAAAAAACCATCAGAGAGATTTCGGCCATCATCGGACTTTGTGCATATGCTGCTACTTACTGGG |
| | GAGGTGAAGACCATCCCACTAGTAACAGTCTGAACGCATTGTTTGTGATGCTTGAGATGCTCAATTACGT |
| | GGATTATAACATCATATTCCGGCGTATGAATTGATGAGTTGTACATCTTGACATTTTCTTCTTTCTTCTC |
| | TTCTCCCTTTCCCAGAAACAAACTTTTTTTACCCACTATAAAATAAAATGAGTATACTACCTGTTATATT |
| | TCTTTCTATATTTTTTTATTCTTCATTCGTTCAGACTTTTAACGCGCCTGAATGTATCGACAAAGGGCAA |
| | TATTTTGCATCATTCATGGAGTTAGAAAACGAGCCAGTAATCTTACCATGTCCTCAAATAAATACGCTAT |
| | CATCCGGATATAATATATTAGATATTTTATGGGAAAAACGAGGAGCGGATAATGATAGAATTATACCGAT |
| | AGATAATGGTAGCAATATGCTAATTCTGAACCCGACACAATCAGACTCTGGTATTTATATATGCATTACC |
| | ACGAACGAAACCTACTGTGACATGATGTCGTTAAATTTGACAATCGTGTCTGTCTCAGAATCAAATATAG |
| | ATCTTATCTCGTATCCACAAATAGTAAATGAGAGATCTACTGGCGAAATGGTATGTCCCAATATTAATGC |
| | ATTTATTGCTAGTAACGTAAACGCAGATATTATATGGAGCGGGCATCGACGCCTTAGAAATAAGAGACTT |
| | AAACAACGGACACCTGGAATTATTACCATAGAAGATGTTAGAAAAAATGATGCTGGTTATTATACATGTG |
| | TTTTAGAATATATATACGGTGGCAAAACATATAACGTAACCAGAATTGTAAAATTAGAGGTACGGGATAA |
| | AATAATACCTTCTACTATGCAATTACCAGATGGCATTGTAACTTCAATAGGTAGTAATTTGACTATTGCA |
| | TGCAGAGTATCGTTGAGACCTCCCACAACGGATGCAGACGTCTTTTGGATAAGTAATGGTATGTATTACG |
| | AAGAAGATGATGGGGACGGAAACGGTAGAATAAGTGTAGCAAATAAAATCTATATGACCGATAAGAGACG |
| | TGTTATTACATCCCGGTTAAACATTAATCCTGTCAAGGAAGAAGATGCTACAACGTTTACGTGTATGGCG |
| | TTTACTATTCCTAGCATCAGTAAAACAGTTACTGTTAGTATAACGTGAATGTATGTTGTTACATTTCCAT |
| | GTCAATTGAGTTTATAAGAATTTTTATACATTATCTTCCAACAAGCAATTGACGAACGTATTGCTATGAT |
| | TAACTCCCACGATACTATGCATATTATTAATCATTAACTTGCAGACTATACCTAGAGCTATTTTGACATA |
| | CTCGTGTTCTTGTGTAATTGCAGTATCTATATTATTAAAGTACGTAAATCTAGCTATAGTTTTATTATTT |
| | AATTTTAGATAATATACCGTCTCCTTATTTTTAAAAATTGCCACATCCTTTATTAAATCATGAATGGGAA |
| | TTTCTATGTCATAGTTAATATATTGTGAACAACAAGAGCAGATATCTATAGGAAAGGGTGGAATGCGATA |
| | CATTGATCTATGTAGTTTTAAAACACACGCAAACTTTGAAGAATTTATATAAATCATTCCATCGATACAT |
| | CCTTCTATGTTGAGATGTATATATCCAGGAATTCGTTTATTAATATCGGGAAATGTATAAACTAAAACAT |
| | TGCCCGGAAGCGGAGCTTCTACCGGAGTTATATCAGTTTTTAACTTACAAAATGTAACCAATACCTTTGC |
| | ATGACTTGTTTTGTTCGGCAACGTTAGTTTAAACTTGACGAATGGATTAATTACAATAGCATGATCCGCG |
| | CATCTATTAAGTTTTTTTACTTTAACGCCCTTGTATGTTTTTACAGAGACTTTATCTAAATTTCTAGTGC |
| | TTGTATGTGTTATAAATATAACGGGATATAGAACTGAATCACCTACCTTAGATACCCAATTACATTTTAT |
| | CAGATCCAGATAATAAACAAATTTTGTCGCCCTAACTAATTCTATATTGTTATATATTTTACAATTGGTT |
| | ATGATATCATGTAATAACTTGGAATCTAACGCACATCGTCGTACGTTTATACAATTGTGATTTAGTGTAG |
| | TATATCTACACATGTATTTTTCCGCGCTATAGTATTCTGGACTAGTGATAAAACTATCGTTATATCTGTC |
| | TTCAATGAACTCATCGAGATATTGCTCTCTGTCATATTCATACACCTGCATAAACTTTCTAGACATCTTA |
| | CAATCCGTGTTATTTTAGGATCATATTTACATATTTACGGGTATATCAAAGATGTTAGATTAGTTAATGG |
| | GAATCGTCTATAATAATGAATATTAAACAATTATATGAGGACTTTTACCACAAAGCATCATAAAAATGAG |
| | TCGTCGTCTGATTTATGTTTTAAATATCAACCGCGAATCAACTCATAAAATACAAGAGAATGAAATATAT |
| | ACATATTTTAGTCATTGCAATATAGACCATACTTCTACAGAACTTGATTTTGTAGTTAAAAACTATGATC |
| | TAAACAGACGACAACCTGTAACTGGGTATACTGCACTACACTGCTATTTGTATAATAATTACTTTACAAA |
| | CGATGTACTGAAGATATTATTAAATCATGGAGTGGATGTAACGATGAAAACCAGTAGCGGACGTATGCCT |
| | GTTTATATATTGCTTACTAGATGTTGCAATATTTCACATGATGTAGTGATAGATATGATAGACAAAGATA |
| | AAAACCACTTATTACATAGAGACTATTCCAACCTATTACTAGAGTATATAAAATCTCGTTACATGTTATT |
| | AAAGGAAGAGGATATCGATGAGAACATAGTATCCACTTTATTAGATAAGGGAATCGATCCTAACTTTAAA |
| | CAAGACGGATATACAGCGTTACATTATTATTATTTGTGTCTCGCACACGTTTATAAACCAGGTGAGTGTA |
| | GAAAACCGATAACGATAAAAAAGGCCAAGCGAATTATTTCTTTGTTTATACAACATGGAGCTAATCTAAA |
| | CGCGTTAGATAATTGTGGTAATACACCATTCCATTTGTATCTTAGTATTGAAATGTGTAATAATATTCAT |
| | ATGACTAAAATGCTGTTGACTTTTAATCCGAATTTCGAAATATGTAATAATCATGGATTAACGCCTATAC |
| | TATGTTATATAACTTCCGACTACATACAACACGATATTCTTGTTATGTTAATACATCACTATGAAACAAA |
| | TGTTGGAGAAATGCCGATAGATGAGCGTCGTATAATCGTATTCGAGTTTATCAAAACATATTCTACACGT |
| | CCTGCAGATTCGATAACTTATTTGATGAATAGGTTTAAAAATATAGATATTTATACCCGCTATGAAGGAA |
| | AGACATTATTACACGTAGCATGTGAATATAATAATACACACGTAATAGATTATCTTATACGTATCAACGG |
| | AGATATAAATGCGTTAACCGACAATAACAAACACGCTACACAACTCATTATAGATAACAAAGAAAATTCC |
| | CCATATACCATTAATTGTTTACTGTATATACTTAGATATATTGTAGATAAGAATGTGATAAGATCGTTGG |
| | TGGATCAACTTCCATCTCTACCTATCTTCGATATAAAATCATTTGAGAAATTCATATCCTACTGTATACT |
| | TTTAGATGACACATTTTACAATAGACACGTTAGGAATCGCGATTCTAAAACGTATCGATACGCATTTTCA |
| | AAATACATGTCGTTTGATAAATACGATGGTATAATAACTAAATGTCATAAAGAAACAATATTGCTCAAAC |
| | TATCCACTGTTCTAGACACTACACTATATGCAGTTTTAAGATGCCATAATTCGAAAAAGTTAAGAAGATA |
| | CCTCACCGAGTTAAAAAAATATAATAACGATAAGTCCTTTAAAATATATTCTAATATTATGAATGAGAGA |
| | TACCTTAATGTATATTATAAAGATATGTACGTGTCAAAGGTATATGATAAACTATTTCCTGTTTTCACAG |
| | ATAAAAATTGTCTACTAACATTACTACCTTCAGAAATTATATACGAAATATTATACATGCTGACAATTAA |
| | CGATCTTTATAATATATCGTATCCACCTACCAAAGTATAGTTGTATTTTTCTCATGCGATGTGTGTAAAA |
| | AAACTGATATTATATAAATATTTTAGTGCCGTATAATAAAGATGACGATGAAAATGATGGTACATATATA |
| | TTTCGTATCATTATTGTTATTGCTATTCCACAGTTACGCCATAGACATCGAAAATGAAATCACAGAATTC |
| | TTCAATAAAATGAGAGATACTCTACCAGCTAAAGACTCTAAATGGTTGAATCCAGCATGTATGTTCGGAG |
| | GCACAATGAATGATATAGCCGCTCTAGGAGAGCCATTCAGCGCAAAGTGTCCTCCTATTGAAGACAGTCT |
| | TTTATCGCACAGATATAAAGACTATGTGGTTAAATGGGAAAGGCTAGAAAAAAATAGACGGCGACAGGTT |
| | TCTAATAAACGTGTTAAACATGGTGATTTATGGATAGCCAACTATACATCTAAATTCAGTAACCGTAGGT |
| | ATTTGTGCACCGTAACTACAAAGAATGGTGACTGTGTTCAGGGTATAGTTAGATCTCATATTAGAAAACC |
| | TCCTTCATGCATTCCAAAAACATATGAACTAGGTACTCATGATAAGTATGGCATAGACTTATACTGTGGA |
| | ATTCTTTACGCAAAACATTATAATAATATAACTTGGTATAAAGATAATAAGGAAATTAATATCGACGACA |
| | TTAAGTATTCACAAACGGGAAAGGAATTAATTATTCATAATCCAGAGTTAGAAGATAGCGGAAGATACGA |
| | CTGTTACGTTCATTACGACGACGTTAGAATCAAGAATGATATCGTAGTATCAAGATGTAAAATACTTACG |
| | GTTATACCGTCACAAGACCACAGGTTTAAACTAATACTAGATCCAAAAATCAACGTAACGATAGGAGAAC |
| | CTGCCAATATAACATGCACTGCTGTGTCAACGTCATTATTGATTGACGATGTACTGATTGAATGGGAAAA |
| | TCCATCCGGATGGCTTATAGGATTCGATTTTGATGTATACTCTGTTTTAACTAGTAGAGGCGGTATTACC |
| | GAGGCGACCTTGTACTTTGAAAATGTTACTGAAGAATATATAGGTAATACATATAAATGTCGTGGACACA |
| | ACTATTATTTTGAAAAAACCCTTACAACTACAGTAGTATTGGAGTAAATATACAATGCATTTTTATATAC |
| | ATTACTGAATAATTATTATTATTATTTATATCGTATTTGTGCTATAACGCGACTATCTAGGTATTTGTAT |
| | CTCACCGATAGAGAACATATAAATGTAGACTCTATTAAACAGTTGTGTAAAATATCAGATCCTAATGCAT |
| | GTTATAGATGTGGATGTACGGCTTTACATGAGTACTTTTATAATTATAGATCAGTCAACGGAAAATACAA |
| | GTATAGATACAACGGTTACTATCAATATTATTTATCTAGCGATTATGAAAATTATAATGAATATTATTAT |
| | GATGATTATGATAGAACTGGTATGAACAGTGAGAGTGATAATATATCAATCAAAACAGAATATGAATTCT |
| | ATGATGAAACACAAGATCAAAGTACACAACTAGTAGGTTACGACATTAAACTCAAAACCAATGAGGATGA |
| | TTTTATGGCTATGATAGATCAGTGGGTGTCCATGATTATATAGATGAATCAATTAATAAAGTAGTATATG |
| | GAAGAGAGTCTCACGTAAGATGGCGGGATATATGGCAAGAACATAATGATGGCGTATACAGTATAGGAAA |
| | GGAGTGCATAGATAATATATACGAAGACAACCATACCGTAGACGAATTCTACAAGATAGACAGCGTATCA |
| | GATGTAGATGACGCGGAACACATATCTCCGATAACTAATGATGTATCTACACAAACATGGGAAAAGAAAT |
| | CAGAGTTAGATAGATACATGGAAATGTATCCTCGTCATAGATATAGTAAGCATTCTGTCTTTAAGGGATT |
| | TTCTGACAAAGTTAGAAAAAATGATTTAGACATGAATGTGGTAAAAGAATTACTTTCTAACGGTGCATCT |
| | CTAACAATTAAGGATAGCAGTAATAAGGATCCAATAACCGTTTATTTTCGAAGAACGATAATGAATTTAG |
| | AAATGATTGATGAACGAAAGTATATAGTACACTCCTATCTAAAAAATTATAAAAATTTCGATTATCCATT |
| | TTTCAGGAAGTTAGTTTTGACTAATAAACATTGTCTCAACAATTATTATAATATAAGCGACAGCAAATAT |
| | GGAACACCGCTACATATATTGGCGTCTAATAAAAAATTAATAACTCCTAATTACATGAAGTTATTAGTGT |
| | ATAACGGAAATGATATAAACGCACGAGGTGAAGATACACAAATGCGAACTCCATTACACAAATATTTGTG |
| | TAAATTTGTATATCATAATATTGAATATGGTATCCGATACTATAATGAAAAGATTATAGACGCATTTATA |
| | GAGTTAGGAGCCGATCTAACTATTCCAAATGACGATGGAATGATACCAGTAGTTTACTGTATACACTCAA |
| | ATGCCGAATATGGTTATAACAATATTACTAACATAAAGATAATACGTAAACTACTTAATCTTAGTAGACG |
| | TGCGTCACATAATCTATTTAGAGATCGAGTCATGCACGATTATATAAGTAATACATATATTGATCTTGAG |
| | TGTTTAGATATTATTAGATCGTTGGATGGATTCGATATCAATGGTTACTTTGAAGGACGTACACCACTTC |
| | ATTGCGCTATACAACATAACTTCACTCAGATTGCTAAGTACTTATTAGATCGAGGAGCTGATATAGTCGT |
| | ACCCAACACATTGATTATACATCAGTACATACAGTAAATAGCATAGATATGGAGGAGGATACAAATATTT |
| | CAAATAAAGTTATAAGGTACAACACTGTCAATAATATATGGGAAACATTACCTAACTTCTGGACTGGAAC |
| | TATAAATCCAGGCGTGGTCTCGCATAAAGATGATATATATGTTGTATGCGACATCAAAGATGAAAAAAAT |
| | GTTAAAACTTGTATATTTAGATATAACACGAATACGTATAACGGATGGGAATTGGTCACGACGACAGAAA |
| | GCAGATTATCAGCTCTGCATACTATTCTTTATAACAATACCATAATGATGTTACATTGTTATGAATCGTA |
| | TATGTTACAAGATACATTTAATGTGTACACTCGCGAATGGAATCATATGTGTCATCAACATTCGAATAGT |
| | TATATCATGTACAATATACTACCCATCTACTAAATATAATAGAATAAAATAAATGAGTATGATCATTTTA |
| | GATAACGATTGATTTTATCATTACCGCTTCATTCTTATATTCTTTGCTTACGGAACCTATATTTAGAAAC |
| | ATCTACTAACGATTTTTTATGCTTGCATTATTAATGGTATGTAATATGATTGATTGTGTACGCAATACCA |
| | ATTTGTTAAGTATGAATACGGGGTACAAACATAAACTGAAGTTTAACATTATTTATTTATGATATATATC |
| | GTTATTGTTTGGTCTATACCATGGATATCTTTAAAGAACTAATCTTAAAACACACGGATGAAAATGTTTT |
| | GATTTCTCCAGTTTCTATTTTATCTACTTTATCTATTCTAAATCATGGAGCAGCTGGTTCTACAGCTGAA |
| | CAACTATCAAAATATATAGAGAATATGAATGAGAATACACCCGATGACAATAATGACATGGACGTAGATA |
| | TTCCGTATTGTGCGACACTAGCTACCGCAAATAAAATATACGGTAGCGATAGTATCGAGTTCCACGCCTC |
| | CTTCCTACAAAAAATAAAAGACGATTTTCAAACTGTAAACTTTAATAATGCTAACCAAACAAAGGAACTA |
| | ATCAACGAATGGGTTAAGACAATGACAAATGGTAAAATTAATTCCTTATTGACTAGTCCGCTATCCATTA |
| | ATACTCGTATGACAGTTGTTAGCGCCGTCCATTTTAAAGCAATGTGGAAATATCCATTTTCTAAACATCT |
| | TACATATACAGACAAGTTTTATATTTCTAAGAATATAGTTACCAGTGTTGATATGATGGTGAGCACTGAG |
| | AATAACTTGCAATATGTACATATTAATGAATTATTCGGAGGATTCTCTATTATCGATATTCCATACGAGG |
| | GAAACTCTAGTATGGTAATTATACTACCGGACGACATAGAAGGTATATATAACATAGAAAAAAATATAAC |
| | AGATGAAAAATTTAAAAAATGGTGTGGTATGTTATCTACTAAAAGTATAGACTTGTATATGCCAAAGTTT |
| | AAAGTGGAAATGACAGAACCGTATAATCTGGTACCGATTTTAGAAAATTTAGGACTTACTAATATATTCG |
| | GATATTATGCAGATTTTAGCAAGATGTGTAATGAAACTATCACTGTAGAAAAATTTCTACATACGACGTT |
| | TATAGATGTTAATGAGGAGTATACAGAAGCATCGGCCGTTACAGGAGTATTTATGACTAACTTTTCGATG |
| | GTATATCGTACGAAGGTCTACATAAACCATCCATTCATGTACATGATTAAAGACAACACAGGACGTATAC |
| | TTTTTATAGGGAAATACTGCTATCCGCAATAAATATAAACAAATAGACTTTTATCACGTTTATCTATGTC |
| | TAAATATTACAAATAGTAATAGTATAAACTAAAGCTGATAATACTTAAAAAAATAATAATATCATTTACA |
| | ATTAATAGTATAAACTAAAAATTAAACAAATCGTTATTATAAGTAATATCAAAATGATGATATACGGATT |
| | AATAGCGTGTCTTATATTCGTGACTTCATCCATCGCTAGTCCACTTTATATTCCCGTTATTCCACCCATT |
| | TCGGAAGATAAATCGTTCAATAGTGTAGAGGTATTAGTTTCCTTGTTTAGAGATGACCAAAAAGACTATA |
| | CGGTAACTTCTCAGTTCAATAACTACACTATCGATACCAAAGACTGGACTATCGGCGTACTATCCACACC |
| | TGATGGTTTGGATATACCATTGACTAATATAACTTATTGGTCACGGTTTACTATAGGTCGTGCATTGTTC |
| | AAATCAGAGTCTGAGGATATTTTCCAAAAGAAAATGAGTATTCTAGGTGTTTCTATAGAATGTAAGAAGT |
| | CGTCGACATTACTTACTTTTTTGACCGTGCGTAAAATGACTCGAGTATTTAATAAATTTCCAGATATGGC |
| | TTATTATCGAGGAGACTGTTTAAAAGCCGTTTATGTAACAATGACTTATAAAAATACTAAAACTGGAGAG |
| | ACTGATTACACGTACCTCTCTAATGGGGGGTTGCCTGCATACTATCGTAATGGGGTCGATGGTTGATTAT |
| | TGATTAGTATATTCCTTATTCTTTTTATTCACACAAAAAGAACATTTTTATAAACATGAAACCACTGTCT |
| | AAATGTAATTATGATCTTGATTTATAGATGAAGATCAGCCTTTAGAGGATTTTAACCAGTATGTTTAATA |
| | TGAAAAAAATAAACATAACATATTTTGAGATTAAGCGCTATTGTGCAAGATTATATTAGAATCAAATTAA |
| | TCTTTCATACGAGAAAAATAACGACATACGTCGTCAACAAATTAAACTTTTTATTTATTAGTTAACTAGC |
| | TTATAGAACTTGCTCATTGTTATGTTTCTAAAACGGGTACGACATATAGGACAATTATCCGACGCACCGG |
| | TTTCTCTTCGTGTTTTATGCCATATATTGATGCATGTTATGCAAAATATATGAGTACACGAATCCAATAA |
| | ACCAAAGTATCTATCGTTTTGAGTAAACAACTTCATAGCAAATTCCACATTCTTTTTCTTTACTTACTCT |
| | ATACACGTCCTCGTATTTATCCAGTATTTTGATGATATCCAACTCAGAAATGGTTGTTGTATTATTGGGT |
| | GTATTGGGAGTATAGGTATTATTAGCTATGTACCAATTTACCAACCCTCTTAATATTGATTGAACAATCA |
| | CATCGGTTATCCAATCAATAACCACATTAATAACTAAATTGTAGTGTATATATAGACCATATATGTTTCT |
| | ATTTTTTTGACAGTTACGTATAGTTTCAGTAAGTTTTGATTGTTGTATTCCTGTATCTCTAGATAAGTTA |
| | GTCATATAGTCCCTTCCGGCGATACGTTTTTTCCAAGCCCGAAATTGATTAGCCAAATGTGTATTTATTT |
| | TTGTGATATTGATATAATATGTAATGTTATTAATATTTCGGATAATGCATACTGTTAGTCTTATATCATT |
| | TGGTTCATCTATGTATTGTAATATTGTTACATGATCTATAGATGATGTATTGATTTTGGCAGGATCGAAT |
| | TCCATATCCGCGACTAAACAGTGAAAAAAATGTAAATACTTTTTAAATTTTAAATTAGTAAAACTTTTTT |
| | TTATTTTTTATGATTCCAAAAATACTGAATACAAAGTCCTAAATTATAAATATGGAGATCATACTACCAC |
| | AACTTATTATTATGCATACTCAGCCGGTGTAATAGATAGATATATATAATTCTATTACACCGGCAGACAA |
| | TTACCGATCGGTATTTGTCGTTACCAACATACCGTATAATATGTAATATACAATTCCATAACCCATTGAC |
| | AGTTGTTATACATCAAAATTGCAATTCTTTTGATTACGATGTTATAAGAATGTAGTTAATTGATGTATGA |
| | TGTTAATGTGTCCTCTTTCCTCTTATAACATCGTAATCAAAAACTTTTTTATAATATATACCTAATAATG |
| | TGTCTTAATAGTTCTCGTGATTCGTCAAACAATCATTCTTATAAAATATAATAAAGCAACGTAAAACACA |
| | TAAAAATAAGCGTAACTAATAAGACAATGGATATTTACGACGATAAAGGTCTACAGACTATTAAACTGTT |
| | TAATAATGAATTTGATTGTATAAGGAATGACATCAGAGAATTATTTAAACATGTAACTGATTCCGATAGT |
| | ATACAACTTCCGATGGAAGACAATTCTGATATTATAGAAAATATCAGAAAAATACTATATAGACGATTAA |
| | AAAATGTAGAATGTGTTGACATCGATAGTACAATAACTTTTATGAAATACGATCCAAATGATGATAATAA |
| | GCGTACGTGTTCTAATTGGGTACCCTTAACTAATAACTATATGGAATATTGTCTAGTAATATATTTGGAA |
| | ACACCGATATGTGGAGGCAAAATAAAATTATACCACCCTACAGGAAATATAAAGTCGGATAAGGATATTA |
| | TGTTTGCAAAGACTCTAGACTTTAAATCAAAGAAAGTGTTAACTGGACGTAAAACAATTGCCGTTCTAGA |
| | CATATCCGTTTCATATAATAGATCAATGACTACTATTCACTACAACGACGACGTTGATATAGATATACAT |
| | ACTGATAAAAATGGAAAAGAGTTATGTTATTGTTATATAACAATAGATGATCATTACTTGGTTGATGTGG |
| | AAACTATAGGAGTTATAGTCAATAGATCTGGAAAATGTCTGTTAGTAAATAACCATCTAGGTATAGGTAT |
| | CGTTAAAGATAAACGTATAAGCGATAGTTTTGGAGATGTATGTATGGATACAATATTTGACTTTTCTGAA |
| | GCACGAGAGTTATTTTCATTAACTAATGATGATAACAGGAATATAGCATGGGACACTGATAAACTAGACG |
| | ATGATACAGATATATGGACTCCCGTCACAGAAGATGATTACAAATTTCTTTCTAGACTAGTATTGTATGC |
| | AAAATCTCAATCGGATACTGTATTCGACTATTATGTTCTTACTGGTGATACGGAACCACCCACTGTATTC |
| | ATTTTCAAGGTAACTAGATTTTACTTTAATATGCCGAAATAAAAAATTTTTGTATAATATCTAGAGGTAG |
| | AGGTATTGTTTAGATAAATACAAATAACATAGATACATCGCATACTTAGCATTTTTATAAATATACATAA |
| | GACATACACTTTATACATTTTTTTTGTAAAAATACTCATAAAAAAATTTATAAAAATTATGGCACAACCA |
| | TATCTTGTATAGGTAGTTTAGTTCGTCGAGTGAACCTATAAACAGATAATAGACAACACGTAATAATAAT |
| | AATGCCTACTAATACAAGCATAATACCGGGAGATGGGATATATGACGTTGTAGTGTTTGGGTTTTCTGAA |
| | CGTTGATAGTCTACTAATACTACATGCTGACATCTAATGCCTGTATAACCATGAGAGCATCTACAATACA |
| | TACCGTCAATATCTCTAGCGTGGATACAGTCACCGTGTAAACAATATCCATCTCCCTCTGGACCGCATAA |
| | TCTGATAGCTGGAATATCTGTTGTAGCGTTTGTAATTTCTGGCGATGTCGTTTCGATAGCGTTACCACTA |
| | TCGGCGAATGATCTGATTATCATAGCAGCGAACAACAACATCAGATATTTCATCGACATTTTTGATGGAT |
| | TTTGTGTTTATGCTGTTTCTCAGTGTGTGTTTATGACAAGATTGGGAATTTTATATTATTAATTCAGTAA |
| | TATAAACTAATAATATATTGTTAATTGTGTAAATAATATAAAAATAACAATACAATATTGAATGTGTTGC |
| | TGTTAAAAATGATCATAAACACGGAGTTTATTTTATATGTCTCGCATAAACATTACTAAAAAAATATATT |
| | GTTCTGTTTTTCTTTCACATCTTTAATTATGAAAAAGTAAATCATTATGAGATGGACGAGATTGTACGCA |
| | TCGTTCGCGACAGTATGTGGTACATACCTAACGTATTTATGGACGACGGTAAGAATGAAGGTCACGTTTC |
| | TGTCAACAATGTCTGTCATATGTATTTCACGTTCTTTGATGTGGATACATCGTCTCATCTGTTTAAGCTA |
| | GTTATTAAACACTGCGATCTGAATAAACGAGGTAACTCTCCATTACATTGCTATACGATGAATACACGAT |
| | TTAATCCATCTGTATTAAAGATATTGTTACACCACGGCATGCGTAACTTTGATAGCAAGGATGAAAAAGG |
| | ACACCACTATCAATCGATAACAAGATCTTTGATATACTAACGGACACCATTGATGACTTTAGTAAATCAT |
| | CCGATCTATTGCTGTGTTATCTTAGATATAAATTCAATGGGAGCTTAAACTATTACGTTCTGTACAAAGG |
| | ATCCGACGAGGATGAACTCACTTCTCTTCATTACTACTGTAAACACATATCCACGTTCTACAAAAGCAAT |
| | TATTACAAGTTAAGTCACACTAAGATGCGAGCCGAGAAGCGATTCATCTACGCGATAATAGATTATGGAG |
| | CAAACATTAACGCGGTTACACACTTACCTTCAACAGTATACCAAACATAGTCCTCGTGTGGTGTATGCTC |
| | TTTTATCTCGAGGAGCCGATACGAGGATACGTAATAATCTTGATTGTACACCCATCATGGAACGATTGTG |
| | CAACAGGTCATATTCTCATAATGTTACTCAATTGGCACGAACAAAAGGAAGAAGGACAACATCTACTTTA |
| | TCTATTCATAAAACATAATCAAGGATACACTCTCAATATACTACGGTATCTACTAGATAGGTTCGACATT |
| | CAGAAAGACGAATACTATAATACCGCCTTTCAAAATTGTAACAACAATGTTGCCTCATACATCGGATACG |
| | ACATCAACCTTCCGACTAAAGACGGTATTCGACTTGGTGTTTGAAAACAGAAACATCATATACAAGGCGG |
| | ATGTTGTGAATGACATCATCCACCACAGACTGAAAGTATCTCTACCTATGATTAAATCGTTGTTCTACAA |
| | GATGTCTCTCCCTACGACGATTACTACGTAAAGAAGATAATAGCCTACTGCCTATTAAGGGACGAGTCAT |
| | TCGCGGAACTACATAGTAAATTCTGTTTAAACGAGGACTATAAAAGTGTATTTATGAAAAATATATCATT |
| | CGATAAGATAGATTCCATCATCGTGACATAAGTCGCCTTAAAGAGATTCGAATCTCCGACACCGACCTGT |
| | ATACGGTATCACAGCTATCTTAAAGCCATACATTCAGACAGTCACATTTCATTTCCCATGTACGACGATC |
| | TCATAGAACAGTGCCATCTATCGATGGAGCGTAAAAGTAAACTCGTCGACAAAGCACTCAATAAATTAGA |
| | GTCTACCATCGGTCAATCTAGACTATCGTATTTGCCTCCGGAAATTATGCGCAATATCATCTAAACAGTA |
| | TGTTGTACGAAAAGAACCATTACAAATATTATCCATGATAGAAAGAAAATATCTATATGATTGGAGAAGT |
| | AGGAAACAGGAACAAGACGACGATTACTACATTATTAAATCATGAAGTCCGTATTATACTCGTATATATT |
| | GTTTCTCTCATGTATAATAATAAACGGAAGAGATATAGCACCGCATGCACCATCCGATGGAAAGTGTAAA |
| | GACAACGAATACAAACGCCATAATTTGTGTCCGGGAACATACGCTTCCAGATTATGCGATAGCAAGACTA |
| | ACACACAATGTACGCCGTGTGGTTCGGGTACCTTCACATCTCGCAATAATCATTTACCCGCTTGTCTAAG |
| | TTGTAACGGAAGACGCGACCGTGTAACACGACTCACAATAGAATCTGTGAATGCTCTCCCGGATATTATT |
| | GTCTTCTCAAAGGATCATCCGGATGCAAGGCATGTGTTTCCCAAACAAAATGTGGAATAGGATACGGAGT |
| | ATCCGGAGACGTCATCTGTTCTCCGTGTGGTCTCGGAACATATTCTCACACCGTCTCTTCCGCAGATAAA |
| | TGCGAACCCGTACCCAGTAATACCTTTAACTATATCGATGTGGAAATTAATCTGTATCCAGTTAACGACC |
| | ACTACCGGTCTCAGCGAATCCATCTCAACGTCGGAACTAACTATTACTATGAATCATAAAGACTGTAATC |
| | CCGTATTTCGTGAGGAATACTTCTCCGTCCTTAATAAGGTAGCAACTTCAGGTTTCTTTACAGGAGAAAG |
| | GTGTGCACTCTGAATTTCGAGATTAAATGCAATAACAAAAATTTTTCCTCCAAACAGTTAACGAAAGCAA |
| | AGAATGATGACGGTATCATGCCGCATTCGGAGACTGTCTATCTAGCGTCGACATCTATATACTATATAGT |
| | AATACCAATACTCAAGACTACGAAACTGATACAATCTCTTATCATGTGGGTAATGTTCTCGATGTCGATA |
| | GCCATATGCCCGGTAGTTGCGATATACATAAACTGATCACTAATTCCAAACCCACCCGCTTTTTATAGTA |
| | AGTTTTTCACCCATAAATAATAAATACAATAATTAATTTCTCGTAAAAGTAGAAAATATATTCTAATTTA |
| | TTGCACGGTAAGGAAGTAGAATCATAAAGAACAGTACTCAATCAATAGCAATCATGAAACAATATATCGT |
| | CCTGGCATGCATGTGCCTGCCAGTCTTCAGCAATCATCCTCATCCTCCTCCTCGTGTACGGAAGAAGAAA |
| | ACAAACATCATATGGGAATCGATGTTATTATCAAAGTCACAAAGCAAGACCAAACACCGACCAATGATAA |
| | GATTTGCCAATCCGTAACGGAAATTACAGAGTCCGAGTCAGATCCAGATCCCGAGGTGGAATCAGAAGAT |
| | GATTCCACATCAGTCGAGGATGTAGATCCTCCTACCACTTATTACTCCATCATCGGTGGAGGTCTGAGAA |
| | TGAACTTTGGATTCACCAAATGTCCTCAGATTAAATCCATCTCAGAATCCGCTGATGGAAACACAGTGAA |
| | TGCTAGATTGTCCAGCGTGTCCCCAGGACAAGGTAAGGACTCTCCCGCGATCACTCATGAAGAAGCTCTG |
| | GCTATGATCAAAGACTGTGAGGTGTCTATCGACATCAGATGTAGCGAAGAAGAGAAAGACAGCGACATCA |
| | AGACCCATCCAGTACTCGGGTCTAACATCTCTCATAAGAAAGTGAGTTACGAAGATATCATCGGTTCAAC |
| | GATCGTCGATACAAAATGTGTCAAGAATCTAGAGTTTAGCGTTCGTATCGGAGACATGTGCAAGGAATCA |
| | TCTGAACTTGAGGTCAAGGATGGATTTAAGTATGTCGACGGATCGGCATCTGAAGGTGCAACCGATGATA |
| | CTTCACTCATCGATTCAACAAAACTCAAAGCGTGTGTCTGAATCGATAACTCTATTCATCTGAAATTGGA |
| | TGAGTAGGGTTAATCGAACGATTCAGGCACACCACGAATTAAAAAAGTGTACCGGACACTATATTCCGGT |
| | TTGCAAAACAAAAATGTTCTTAACTACATTCACAAAAAGTTACCTCTCGCGACTTCTTCTTTTTCTGTCT |
| | CAATAGTGTGATACGATTATGACACTATTCCTATTCCTATTCCTATTTCCTTTCAGGGTATCACAAAAAT |
| | ATTAAACCTCTTTCTGATGGTCTCATAAAAAAAGTTTTACAAAAATATTTTTATTCTCTTTCTCTCTTTG |
| | ATGGTCTCATAAAAAAAGTTTTACAAAAATATTTTTATTCTCTTTCTCTCTTTGATGGTCTCATAAAAAA |
| | AGTTTTACAAAAATATTTTTATTCTCTTTCTCTCTTTGATGGTCTCATAAAAAAAGTTTTACAAAAATAT |
| | TTTTATTCTCTTTCTCTCTTTGATGGTCTCATAAAAAAAGTTTTACAAAAATATTTTTATTCTCTTTCTC |
| | TCTTTGATGGTCTCATAAAAAAAGTTTTACAAAAATATTTTTATTCTCTTTCTCTCTTTGATGGTCTCAT |
| | AAAAAAAGTTTTACAAAAATATTTTTATTCTCTTTCTCTCTTTGATGGTCTCATAAAAAAAGTTTTACAA |
| | AAATATTTTTATTCTCTTTCTCTCTTTGATGGTCTCATAAAAAATATTAAACCTCTTTCTGATGGTGTCA |
| | CTAAAATATTTTTATTCTCATTCTCATTTTCTCTTTCTCTCTTCAATGGAGTCATAAAATATTTTTATTC |
| | TCTTTCTCTCTTCGATGGTCTCACAAAAATATTAAACCTCTTTCTGATGGTGTCACTAAAATATTTTTAT |
| | TCTCATTCTCATTTTCTCTTTCTCTCTTCAATGGAGTCATAAAATATTTTTATTCTCTTTCTCTCTTCGA |
| | TGGTCTCACAAAAATATTAAACCTCTTTCTGATGGAGTCGTAAAAAAGTTTTATCTCTTTCTCTCTTCGA |
| | TGGTCTCACAAAAATATTAAACCTCTTTCTGATGGAGTCGTAAAAAAGTTTTATCTCTTTCTCTCTTCGA |
| | TGGTCTCACAAAAATATTAAACCTCTTTCTGATGGAGTCGTAAAAAAGTTTTATCTCTTTCTCTCTTCGA |
| | TGGTCTCACAAAAATATTAAACCTCTTTCTGATGGAGTCGTAAAAAAGTTTTATCTCTTTCTCTCTTCGA |
| | TGGTCTCACAAAAATATTAAACCTCTTTCTGATGGAGTCGTAAAAAAGTTTTATCTCTTTCTCTCTTCGA |
| | TGGTCTCACAAAAATATTAAACCTCTTTCTGATGGAGTCGTAAAAAAGTTTTATCTCTTTCTCTCTTCGA |
| | TGGTCTCACAAAAATATTAAACCTCTTTCTGATGGAGTCGTAAAAAAGTTTTATCTCTTTCTCTCTTCGA |
| | TGGTCTCACAAAAATATTAAACCTCTTTCTGATGGAGTCGTAAAAAAGTTTTATCTCTTTCTCTCTTCGA |
| | TGGTCTCACAAAAATATTAAACCTCTTTCTGATGGAGTCGTAAAAAAGTTTTATCTCTTTCTCTCTTCGA |
| | TGGTCTCACAAAAATATTAAACCTCTTTCTGATGGAGTCGTAAAAAAGTTTTATCTCTTTCTCTCTTCGA |
| | TGGTCTCACAAAAATATTAAACCTCTTTCTGATGGAGTCGTAAAAAAGTTTTATCTCTTTCTCTCTTCGA |
| | TGGTCTCACAAAAATATTAAACCTCTTTCTGATGGAGTCGTAAAAAAGTTTTATCTCTTTCTCTCTTCGA |
| | TGGTCTCACAAAAATATTAAACCTCTTTCTGATGGAGTCGTAAAAAAGTTTTATCTCTTTCTCTCTTCGA |
| | TGGTCTCACAAAAATATTAAACCTCTTTCTGATGGAGTCGTAAAAAAGTTTTATCTCTTTCTCTCTTCGA |
| | TGGTCTCACAAAAATATTAAACCTCTTTCTGATGGAGTCGTAAAAAAGTTTTATCTCTTTCTCTCTTCGA |
| | TGGTCTCACAAAAATATTAAACCTCTTTCTGATGGAGTCGTAAAAAAGTTTTATCTCTTTCTCTCTTCGA |
| | TGGTCTCACAAAAATATTAAACCTCTTTCTGATGGAGTCGTAAAAAAGTTTTATCTCTTTCTCTCTTCGA |
| | TGGTCTCACAAAAATATTAAACCTCTTTCTGATGGAGTCGTAAAAAAGTTTTATCTCTTTCTCTCTTCGA |
| | TGGTCTCACTAAAATATTTTTTATTCTCTTTCTGATGCATCAACTATTTCTTAAACAATAACGTCCAACA |
| | ACATATACTCGTCGAGCTTATCAACATCCCCTATGCCCATCTAGGTTACCAGACAATTGTATATCATAAA |
| | ATAATGTTTATAATTTACACGTTAAAATCATATAATAAAACGTAGATCGTATAATATTTTTTGGTATATA |
| | AATGATCTAGTAAAATCCATGTAGGGGATACTGCTCACATTTTTTCTTTGGTACAAAATTTCACACAAGT |
| | TTTTATACAGACAAATTCTTGTCCATATATTTTAAAACATTGACTTTTGTACTAAGAAAAATATCTAGAT |
| | CAACTATCTCTTTCTCTTTCTCTCTTCGATGGTCTCACAAAAATATTAAACCTCTTTCTGATGGAGTCGT |
| | AAAAAAGTTTTATCTCTTTCTCTCTTCGATGGTCTCACAAAAATATTAAACCTCTTTCTGATGGAGTCGT |
| | AAAAAAGTTTTATCTCTTTCTCTCTTCGATGGTCTCACAAAAATATTAAACCTCTTTCTGATGGAGTCGT |
| | AAAAAAGTTTTATCTCTTTCTCTCTTCGATGGTCTCACAAAAATATTAAACCTCTTTCTGATGGAGTCGT |
| | AAAAAAGTTTTATCTCTTTCTCTCTTCGATGGTCTCACAAAAATATTAAACCTCTTTCTGATGGAGTCGT |
| | AAAAAAGTTTTATCTCTTTCTCTCTTCGATGGTCTCACAAAAATATTAAACCTCTTTCTGATGGAGTCGT |
| | AAAAAAGTTTTATCTCTTTCTCCTTCGATGGTCTCACAAAAATATTAAACCTCTTTCTGATGGAGTCGTA |
| | AAAAAGTTTTATCTCTTTCTCTCTTCGATGGTCTCACAAAAATATTAAACCTCTTTCTGATGGAGTCGTA |
| | AAAAAGTTTTATCTCTTTCTCTCTTCGATGGTCTCACAAAAATATTAAACCTCTTTCTGATGGAGTCGTA |
| | AAAAAGTTTTATCTCTTTCTCCTTCGATGGTCTCACAAAAATATTAAACCTCTTTCTGATGGAGTCGTAA |
| | AAAAGTTTTATCTCTTTCTCCTTCGATGGTCTCACAAAAATATTAAACCTCTTTCTGATGGAGTCGTAAA |
| | AAAGTTTTATCTCTTTCTCTCTTCGATGGTCTCACAAAAATATTAAACCTCTTTCTGATGGAGTCGTAAA |
| | AAAGTTTTATCTCTTTCTCTCTTCGATGGTCTCACAAAAATATTAAACCTCTTTCTGATGGTCTCTATAA |
| | AGCGATCGATCTTTCTTACACTCTAGAGTTTCCTACAGTCATGGGTCACACATTTTTTTCTAGACACTAA |
| | ATAAAATTAGTAAAATTAAATTAATTATAAAATTATATATATAATTTACTAACTTTAGTTAGATAAATTA |
| | ATAATATATAAGTTTTAGTACATTAATATTATATTTTAAAT |

In some embodiments, the oncolytic virus (e.g., a producer virus, engineered producer virus as described herein) can comprises various exogenous nucleic acids, alone, or in combination with viral gene modifications, such as mutation or deletion (complete or partial deletion) or viral genes. Non-limiting examples of viral genes can include: B5R, A52R (VACWR178), F13L, A36R, A34R, A33R, B8R, B18R, SPI-1, SPI-2, B15R, VGF, E3L, K3L, A41L, K7R, N1L, C12L, TK, and any combinations thereof. Non-limiting examples of exogenous nucleic acids can include nucleic acid sequences coding for: CXCR4, CCR2, PH-20, HMGB1, PIAS3, IL15, IL15-Rα, LIGHT, ITAC, fractalkine, CCL5, a metabolic modulating protein, a cytokine, a fusion protein comprising any combinations of the above, and a functional domain or fragment or variant thereof, or any combinations thereof

Hyaluronan (HA) is an important structural element of ECM and a high molecular weight linear glycosaminoglycan consisting of repeating disaccharide units. It can be distributed widely throughout connective, epithelial, and neural tissues, and its expression level can be significantly elevated in many types of tumors. Hyaluronidases are a family of enzymes that catalyze the degradation of HA. There are at least five functional hyaluronidases identified so far in human: HYAL1, HYAL2, HYAL3, HYAL4 and HYAL5 (also known as PH-20 or SPAM1), among which PH-20 is the only one known so far to be functional at relatively neutral pH. In some embodiments of the present disclosure, combining hyaluronidase with other tumor-targeting therapeutic agents (such as transgenes, also referred to herein as exogenous nucleic acid) can promote the therapeutic effect of the modified oncolytic viruses that can be used as producer viruses as described herein, at least by diminishing the ECM and enhancing the transportation of the therapeutic agent inside and between the tumors.

Some embodiments herein disclose a modified oncolytic virus that can be used as a producer virus as described herein, that can comprise an exogenous nucleic acid coding for a membrane associated protein that is capable of degrading hyaluronan, such as a hyaluronidase. It should be noted that the term “hyaluronidase” as used herein can refer to any enzyme or a fragment thereof that catalyzes the degradation of HA in a tumor, including, but not limited to, PH-20 and its homologs from other species, as well as other engineered/design proteins with similar enzymatic function. As used herein, hyaluronidase can refer to a class of hyaluronan degrading enzymes.

In some embodiments, the modified oncolytic virus that can be used as a producer virus as described herein can comprises an exogenous nucleic acid that can code for a chemokine receptor that is a chimeric protein. At least part of its extracellular domain can be from a chemokine receptor that promotes the tumor-targeted delivery of the virus, and at least part of its intracellular domain can be from a chemokine receptor that promotes the tumor-specific replication, inhibits immunosuppressive activity, or conveys some other beneficial effects, or vice versa. For instance, the modified oncolytic virus can comprise a nucleic acid that codes for a protein having an intracellular GTPase domain of CCR5, and an extracellular chemokine-binding domain of CXCR4 or CCR2. In some case, by combining domains with different functionalities one may achieve further improvement in therapeutic performance of the modified oncolytic virus. It is one embodiment of this disclosure that the modified oncolytic virus can comprise exogenous nucleic acids that can code for at least one chemokine receptor. In some cases, the modified oncolytic virus can comprise exogenous nucleic acids that can code for two or more different chemokine receptors, which may be expressed simultaneously by the virus. Exemplary chemokine receptors that can be expressed simultaneously from the modified oncolytic viruses described herein can include CXCR4 and CCR2. In modified oncolytic viruses expressing more than one chemokine receptors, a combinatorial or synergistic effect against tumor cells may be achieved as to the therapeutic application of the oncolytic virus.

In certain embodiments, the modified oncolytic virus comprises an exogenous CXCR4-expressing nucleic acid. In certain embodiments, the modified oncolytic virus comprises an exogenous CCR2-expressing nucleic acid. Certain embodiments disclose a modified oncolytic virus comprising an exogenous nucleic acid that codes for both CXCR4 and CCR2, and both chemokines are expressed form the same virus. Under certain circumstances, CXCL12 and/or CCL2 typically expressed in the tumor microenvironment may attract the CXCR4 and/or CCR2-expressing lymphocytes or other migrating cells that are infected by the modified oncolytic virus, thereby enhancing the tumor-targeted delivery of the modified oncolytic virus.

In certain embodiments, producer viruses described herein can comprise one or more exogenous nucleic acid sequences, alternatively referred to as transgenes, which can generate mRNAs coding for an agent that can modulate the activity of STAT3 and as a result can also modulate the activation of genes regulated by STAT3. Thus, certain examples provided herein provide oncolytic vaccinia viruses containing exogenous nucleic acid sequences that can encode an agent that can modulate STAT-3 mediated gene-activation. The phrase “modulates STAT 3-mediated gene activation,” as used herein, can refer to a process wherein STAT3 activity is modulated and as a consequence the activation of one or more genes that are regulated by STAT3 is also modulated.

In certain embodiments, the agent that can modulate STAT3-mediated gene activation can be a protein or a fragment thereof. In certain embodiments, the protein or the fragment thereof can inhibit, reduce, or minimize STAT3 activity and STAT3-mediated gene activation. A protein or a fragment thereof that inhibits, reduces and/or minimizes STAT3 activity and STAT3-mediated gene activation can, for example, block the binding of STAT3 to a DNA binding sequence in the promoter regions of STAT3 responsive genes. In additional examples, the protein or a fragment thereof that inhibits, reduces, or minimizes STAT3 activity and STAT3-mediated gene activation can directly bind the STAT3 protein, for example, at the SH2 domain. In certain embodiments, a protein that inhibits, reduces and/or minimizes STAT3 activity blocks, prevents, reduces and/or minimizes the phosphorylation of STAT3 and/or dephosphorylates STAT3. In certain non-limiting embodiments, the proteins that modulate STAT3 activity can include phosphotyrosine phosphatases (PTPs), protein inhibitor of activated STAT (PIAS, e.g., PIAS3) and suppressor of cytokine signaling (SOCS) proteins (e.g., SOC3).

For example, in some aspects, a vaccinia virus genome can comprise an exogenous env, packaging construct, and a transfer construct of a lentivirus. Exogenous sequences can be inserted at any portion of a vaccinia virus genome. In an aspect, an exogenous sequence can be inserted in proximity to a 5′ or 3′ end. In an aspect, an exogenous sequence is inserted adjacent to another exogenous sequence. In an aspect, an exogenous sequence is inserted from about 0.5 kb to about 1000 kb away from the next nearest exogenous sequence. In some instances, the exogenous sequences coding for the lentivirus proteins can be under the control of early vaccinia virus promoters. In some embodiments, the producer virus can be an oncolytic vaccinia virus which expresses a bacteriophage regulatory sequence, such as a bacteriophage promoter, a bacteriophage termination sequence, or combinations thereof. Bacteriophage promoters or termination sequences can be from T3 bacteriophage, T7 bacteriophage and SP6 bacteriophage. The bacteriophage regulatory sequences can be cloned using standard methods for cloning and manipulating. In some embodiments, the producer virus can be an oncolytic vaccinia virus which expresses a bacteriophage polymerase. The term “bacteriophage polymerase” can refer to any bacteriophage polymerase, including those compatible with T3 bacteriophage, T7 bacteriophage and SP6 bacteriophage promoters. In some embodiments, the producer virus can be an oncolytic vaccinia virus which expresses a bacteriophage polymerase and the bacteriophage polymerase can be cloned using standard methods of cloning and manipulating.

### Env Element

In some aspects, an exogenous sequence can be an env element. In some aspects, an env element can be from a vsvg-containing env construct. In some aspects, a vsvg-containing env construct may recombine with the vaccinia virus genome at a point between the vacwr032 (k11) and vacwr033 (k21) genes. In an aspect, a vsvg-containing env construct can be designed to recombine with a vaccinia virus genome at a point between the vacwr032 (k11) and vacwr033 (k21) genes. This genomic location can offer a few advantages: (1) it provides adequate space between genes, (2) an identifiable promoter could be found for vacwr032, and (3) this positions the construct at the 5′ end of the genome which is the furthest away from any other exogenous sequences that can disrupt an endogenous vaccinia virus genome. In an aspect, the vsvg gene and its promoter can be positioned 3′ to 5′ to match the direction of the adjacent VV genes. In some cases, an env is inserted at position bp 26041 of a vaccinia genome. In an aspect, an envelope construct can be inserted between genes vacwr032 and vacwr033 of an oncolytic vaccinia virus. In an aspect, an envelope construct can be inserted between genes vacwr032 or a portion thereof and vacwr033 or a portion thereof of the oncolytic vaccinia virus.

In an aspect, an env element comprises a sequence with at least about 50%, 60%, 70%, 80%, 85%, 90%, 95%, 96%, 97%, 98%, 99%, or 100% identity to SEQ ID NO: 2. In an aspect, an env element comprises a vsvg sequence with at least about 50%, 60%, 70%, 80%, 85%, 90%, 95%, 96%, 97%, 98%, 99%, or 100% identity to SEQ ID NO: 3.

### Packaging Construct

In an aspect, an exogenous sequence can be a packaging construct sequence. In an aspect, a packaging construct can be designed to recombine with a vaccinia virus genome to replace the coding sequence for a gene, for example vacwr094 (j2r) between the vacwr093 (j1r) and vacwr033 (j3r) genes. In an aspect, a genomic disruption caused by an insertion of an exogenous sequence can be performed. In an aspect, a genomic disruption can attenuate a function of a virus. For example, a packaging construct can be designed to recombine with a vaccinia virus genome to replace or disrupt the coding sequence for a gene because a disruption, for example aj2r-deletion, creates an attenuated virus that preferentially infects tumor cells. Also, this disruption/insertion positions the exogenous packaging construct near the center of the vaccinia virus genome which is the relatively distant from the other exogenous sequences, such as env. In some cases, a packaging construct is inserted or deleted at position bp 80724-81257 of a vaccinia virus genome. In an aspect, a packaging construct can be inserted between genes vacwr093 and vacwr095 of an oncolytic vaccinia virus. In an aspect, a genome of an oncolytic vaccinia virus comprises a deletion of the vacwr094 gene or portion thereof. In an aspect, a packaging construct can be inserted into the location of the vacwr094 gene or portion thereof. In an aspect, a packaging construct can be inserted between genes vacwr093 or a portion thereof and vacwr095 or a portion thereof of an oncolytic vaccinia virus. In an aspect, a genome of an oncolytic vaccinia virus comprises a deletion of a vacwr094 gene or portion thereof. In an aspect, a packaging construct can be inserted into the location of the vacwr094 gene or portion thereof.

In an aspect, an packaging element comprises a sequence with at least about 50%, 60%, 70%, 80%, 85%, 90%, 95%, 96%, 97%, 98%, 99%, or 100% identity to SEQ ID NO: 4. In an aspect, an packaging element comprises a gag/pol sequence with at least about 50%, 60%, 70%, 80%, 85%, 90%, 95%, 96%, 97%, 98%, 99%, or 100% identity to SEQ ID NO: 5. In an aspect, the nucleic acid that codes for the lentiviral structural protein comprises a nucleotide sequence as set forth in SEQ ID No. 4, or a nucleotide sequence that is at least about 70% to at least about 99% identical to the sequence set forth as SEQ ID No. 5

### Transfer Construct

In an aspect, an exogenous sequence can be a transfer construct sequence. In an aspect, vaccinia virus late genes may not be transcribed into single gene-containing mRNA, but instead from large 10-20 kb mRNA that may have been initiated using promoters several genes away before terminating. In an aspect, a disrupted vaccinia virus genome may comprise a transfer construct between vacwr205 and vacwr206, a region with no late gene transcripts near the far 3′ end of the VV genome. In some cases, a transfer construct is inserted at bp 183645 of a vaccinia virus genome. In an aspect, a transfer construct can be inserted between genes vacwr205 or portion thereof and vacwr206 or portion thereof of an oncolytic vaccinia virus.

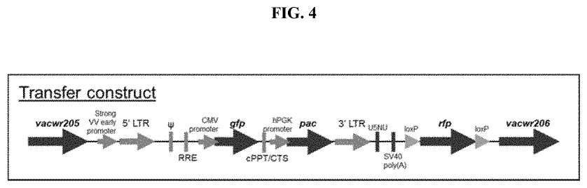

In an aspect, a vaccinia virus provided herein can comprise at least 1 promoter. In an aspect, a vaccinia virus provided herein can comprise from about 1 promoter to about 3 promoters. In an aspect, a vaccinia virus provided herein can comprise from about 1, 2, 3, 4, 5, 6, 7, 8, 9, or up to about 10 promoters. In an aspect, a promoter can be an early promoter. In an aspect, a promoter can be an early/late promoter or a later promoter. In some cases, the transfer construct can further comprise a gene that codes for a self-cleaving RNA ribozyme, such as a hepatitis delta virus ribozyme or a hammerhead ribozyme. Without being bound by any particular theory, it is contemplated that when a self-cleaving RNA sequences are transcribed, they can cleave themselves rapidly and during transcription. Thus, the system may not have to rely on early expression to terminate the RNA as they would likely rapidly cleave themselves at the correct spot. As such, using a late promoter (e.g., a vaccinia virus late promoter) can be advantageous, because now all of the retroviral (e.g., lentiviral) components can be expressed with the late promoters, such as the VV late promoters, which have magnitudes higher expression, and can produce markedly more retrovirus (e.g., lentivirus). The gene that codes for the self-cleaving ribozyme, can, in some instances be positioned 3′ of the termination sequence (e.g., U5NU in FIG. 4) of the transfer construct.

In an aspect, a transfer construct can be inserted between genes vacwr205 or a portion thereof and vacwr206 or a portion thereof of the oncolytic vaccinia virus. In an aspect, a transfer element comprises sequence with at least about 50%, 60%, 70%, 80%, 85%, 90%, 95%, 96%, 97%, 98%, 99%, or 100% identity to SEQ ID NO: 6.

### Lentivirus

In an aspect, provided herein can be an oncolytic virus, such as a vaccinia virus, that produces from its genome a non-replicating lentivirus or portion thereof. In some embodiments, a non-replicating lentivirus comprises a transgene that codes for a non-lentiviral protein or functional fragment thereof. In an aspect, a non-replicating lentivirus is capable of infecting and transducing both dividing and non-dividing cells in a target population of mammalian cells. In an aspect, mammalian cells can be human.

In an aspect, provided herein can be a second-generation lentivirus. A second-generation lentivirus may comprise a transfer plasmid that may utilize a tat element for packaging. In an aspect, a second-generation lentivirus may comprise packaging elements in a single plasmid, for example: Gag, pol, rev, and tat. In an aspect, a second-generation lentivirus may comprise an env element that can encode for VSV-G. A second-generation lentivirus may be a 3-plasmid system. In an aspect, a second-generation lentivirus may utilize a wild type LTR. Second generation lentiviruses can be replication incompetent and may utilize 3 plasmids to encode various HIV genes.

In an aspect, provided herein can be a third-generation lentivirus. A third-generation lentivirus may utilize a second generation or third generation packaging system to package a transfer element. In an aspect, a third-generation lentivirus utilizes at most 2 plasmids to package, for example one plasmid encoding gag and pol and a second plasmid encoding rev. In an aspect, a third-generation lentivirus may comprise an env element that can encode for VSV-G. In an aspect, a third-generation lentivirus uses up to 4 plasmids, and may eliminate the use of a tat element. In an aspect, a third-generation lentivirus may be safer than a second-generation lentivirus. A third-generation lentivirus may comprise a hybrid LTR viral promoter, for example a 5′LTR can be deleted or partially deleted and can be fused to an exogenous promoter, for example a vaccinia virus, CMV or RSV promoter.

In some cases, provided herein can be a lentivirus or a portion thereof. In some cases, a lentivirus can be derived from an HIV-1 virus. In some aspects, a lentivirus can be derived from a non-HIV-1 virus, for example: HIV-2, simian immunodeficiency virus, feline immunodeficiency virus, bovine immunodeficiency virus, or caprine arthritis-encephalitis virus, equine infectious anemia virus (EIAV), and any combination thereof.

In some cases, provided herein can be a lentivirus from an HIV-1 virus. In some aspects, multiple plasmids coding for different components of an HIV-1 virus can be delivered on multiple plasmids. A lentiviral vector system can comprise at least two plasmids that comprise portions of the lentivirus. In some aspects, at least 2, 3, 4, 5, 6, 7, or up to about 8 plasmids can comprise portions of the lentivirus. In some aspects, an LV vector can be a 3-plasmid system. In some cases, a 3-plasmid system comprises two helper plasmids coding for gag-pol and the env functions as well as the TV plasmid. In some aspects, an LV vector can be a 4-plasmid system. 4 plasmid LV systems can comprise accessory genes of HIV-1 (for example: vif, vpr, vpu, and nef), may be present only in a first generation of LV. In some aspects, they have been removed because they may not be necessary. Similarly, the regulatory tat gene, present in the second-generation LV, has been eliminated in some cases because its transacting function is dispensable as the U3 promoter of the 5′ long terminal repeat (LTR) in the TV has been replaced by a constitutively active promoter sequence. In some aspects, a vaccinia virus may be absent a gene from a foreign virus, such as an HIV-1 rev gene. In some aspects, a vector provided herein may contain a hybrid 5′ LTR region. A hybrid 5′ LTR region may contain at least one component from a vaccinia virus and at least one component from an HIV-1. In some aspects, a component from an HIV-1 is a 5′ LTR. In some aspects, a component from an HIV-1 is a 3′ LTR. A hybrid construct may be absent tat. In some aspects, a hybrid construct may utilize a vaccinia virus transcription factor in place of tat.

In some embodiments, a constitutively active promoter sequence can be any one of: cytomegalovirus (promoter) or Rous Sarcoma Virus (promoter). In some cases, an optional enhancer or an inducible/repressible promoter sequence, such as 7tetO may be included (pRRL (lentivirus transfer vector construct containing chimeric Rous sarcoma virus (RSV)-HIV 5′ LTRs) design or the pCCL design ((CMV)-HIV 5′ LTR). These modifications can lead to a LV vector system with the helper functions based on the use of gag-pol (encoding for the structural proteins and viral enzymes) and rev (encoding for a post-transcriptional regulator) derived from HIV-1 and env. In some aspects, a vector provided herein is absent rev. In some aspects, LV vectors can be pseudotyped with different heterologous envelope glycoproteins. In some aspects, a glycoprotein of the vesicular stomatitis virus (VSV-g) envelope can be utilized due to improved stability during downstream processing as well as its large transduction spectrum. In some aspects, a TV plasmid is genetic material transferred to the target cells and can comprise the LV backbone containing the transgene expression cassette flanked by cis-acting elements required for encapsidation, reverse transcription, and integration. In some aspects, a transfected cell comprises at least a portion of a TV plasmid after infection. In some aspects, provided herein can be a self-inactivating (SIN)-LV vector. A SIN-LV vector comprises a deletion at the U3 element of the 2′LTR. In some aspects, a SIN-LV vector loses the transcriptional capacity of the viral LTR once transferred to the target cells; this can minimize the risk of emergence of replication competent recombinants and avoiding problems linked to promoter interference. In some aspects, provided herein can be a third-generation plasmid system in which tat has been removed from the system. In some aspects, a third-generation vector may replace the activity of the HIV-1 protein TAT by using a chimeric 5′ LTR fused to a heterologous promoter.

In some cases, the termination of vaccinia virus early gene transcription can utilize the termination sequence UUUUUNU (U5NU) (SEQ ID NO: 7) to attract vaccinia virus-specific factors to halt transcription and generate poly (A) tails to protect viral mRNA. In an aspect, a vector provided herein may comprise the ATTTTTAT (SEQ ID NO: 8) sequence adjacent to the desired terminal region of a construct. In some embodiments, the termination sequence comprises a nucleotide sequence with percent identity from about 60%, 70%, 80%, 90%, 95%, or up to about 100% to a sequence as set forth in SEQ ID No. 7.

In certain embodiments, the retroviral vectors provided herein can be replication defective, that is, they are unable to replicate autonomously in a target cell. In some aspects, a replication defective virus is a minimal virus, such that it retains only the sequences of its genome which are necessary for target cell recognition and encapsidating the viral genome. In some cases, a replication defective virus is not infective after introduction into a cell. Use of replication defective viral vectors allows for administration to cells in a specific, localized area, without concern that the vector can infect other cells. Thus, a specific tissue can be specifically targeted.

In an aspect, a lentivirus construct provided herein, for expression from a producer oncolytic virus, may comprise a promoter. In an aspect, expression of a lentiviral element may be expressed early. In an aspect, expression of a lentiviral element may be robust. For example, a vaccinia virus may generate short, single gene mRNA with poly (A) tails during an early phase of infection, for example within 2 hr. post-infection. After that period, viral mRNA from the intermediate and late phases of infection may not be specifically terminated or polyadenylated. To form functional lentivirus transfer RNA, transcription can be initiated and terminated at specific sites adjacent to lentiviral long terminal repeats (LTRs), therefore, lentiviral elements may be expressed robustly in the first 2 hr. post vaccinia virus infection. Therefore, in some cases a vector provided herein can have strong early promoters for each element and fused a strong early promoter to a 5′ LTR of the transfer construct. In an aspect, there are from about 1-10 viral elements with preceding strong early promoters. In an aspect, there are from about 1-5 viral elements with preceding strong early promoters. In an aspect, there are from about 1-3 viral elements with preceding strong early promoters. In some instances, the components of the lentiviral construct can be under the control of early/late, or late viral promoters. For example, the packaging construct, the transfer construct, and the envelope construct each independently comprises an early/late viral promoter or a late viral promoter.

Promoters are sequences of nucleic acid that control the binding of RNA polymerase and transcription factors, and can have a major effect on the efficiency of gene transcription, where a gene may be expressed in the cell, and/or what cell types a gene may be expressed in. Non limiting examples of promoters include a vaccinia virus promoter, cytomegalovirus (CMV) promoter, an elongation factor 1 alpha (EF1α) promoter, a simian vacuolating virus (SV40) promoter, a phosphoglycerate kinase (PGK1) promoter, a ubiquitin C (Ubc) promoter, a human beta actin promoter, a CAG promoter, a Tetracycline response element (TRE) promoter, a UAS promoter, an Actin 5c (Ac5) promoter, a polyhedron promoter, Ca2+/calmodulin-dependent protein kinase II (CaMKIIa) promoter, a GAL1 promoter, a GAL 10 promoter, a TEF1 promoter, a glyceraldehyde 3-phosphage dehydrogenase (GDS) promoter, an ADH1 promoter, a CaMV35S promoter, a Ubi promoter, a human polymerase III RNA (H1) promoter, a U6 promoter, or a combination thereof.

In some embodiments, the disclosure provides a method of eliciting an antitumor immune response in a subject presenting with, or at risk of developing a cancer. In some aspects, a cancer can be metastatic. In an aspect, an oncolytic virus (e.g., HSV, vaccinia virus, or adenovirus), a retrovirus produced from the oncolytic virus, or both can express a therapeutic protein or a diagnostic protein. In an aspect, a therapeutic protein can be a PD-1 binding agent, such as a single chain anti-PD-1 antibody, that antagonizes the activity of PD-1. In other embodiments, the therapeutic protein an agent that antagonizes the binding of the PD-1 ligands to the receptor, e.g., anti-PD-L1 and/or PD-L2 antibodies, PD-L1 and/or PD-L2 decoys, or a soluble PD-1 receptor. In an aspect, PD-1 blockade may also stimulate the anti-tumor immune response by blocking the inactivation of T-cells (CTLs and helper) and B-cells thereby leading to increased cancer killing.

### Packaging Cell Lines

In an aspect, provided herein can be a packaging cell line. The production of replication defective virus can be accomplished through trans-complementation in which packaging cells may be cotransfected with multiple plasmids (for example from about 2, 3, 4, 5 or more plasmids) that together express all of the viral proteins necessary to generate infectious particles, as well as the nucleic acid sequence of interest that will be packaged within them for delivery, for example a viral sequences. While many lentiviral vector systems are based on transduction of two helper plasmids (second generation) with the transfer plasmid, some newer systems (third generation) have the packaging and envelope constructs on three plasmids which are combined with the transfer plasmid and utilized to transfect a packaging cell line. In an aspect, provided herein can be a packaging cell line for use with a retrovirus. In an aspect, a first-generation retroviral packaging cell line can be utilized. First generation retroviral packaging cell lines can be derived from mouse 3T3 cell. In an aspect, a second-generation retroviral vector packaging cell line can be utilized. Non-limiting examples of second-generation retroviral vector packaging cell lines include: human cells like HEK293 (embryonic kidney), TE671 (rhabdomyosarcoma) and HT1080 (fibrosarcoma). Third generation packaging cell lines may be absent unnecessary viral sequences present in second generation packaging plasmids as well as in transfer vectors have been deleted to improve safety.

### Non-Lentiviral Proteins

### Therapeutic Protein or Portion Thereof

In some embodiments, a non-lentiviral protein can be a therapeutic protein or portion thereof. In an aspect, a therapeutic protein or portion thereof can be any one of: immune checkpoint modulator, antibody or portion thereof, Fc fusion protein, anticoagulant, blood factor, bone morphogenetic protein, immunosuppressive agents, immunostimulatory agents, enzyme, growth factor, hormone, interferon, interleukin, thrombolytic, anti-angiogenic, chemotherapeutic, antibiotic, antifungal, antiviral, and any combination thereof.

In an aspect, a therapeutic protein or portion thereof can be an immune checkpoint modulator. In an aspect, an immune checkpoint modulator can inhibit an immune checkpoint. In an aspect, an immune checkpoint modulator can activate an immune checkpoint. An immune checkpoint inhibitor can be a drug that blocks a protein made by some types of immune system cells, such as T cells, and some cancer cells. These immune checkpoint proteins help keep immune responses in check and can keep T cells from killing cancer cells. When these proteins are inhibited, the “brakes” on the immune system are released and T cells are able to kill cancer cells. Examples of checkpoint proteins found on T cells or cancer cells include but are not limited to: PD-1/PD-L1 and CTLA-4/B7-1/B7-2. In an aspect, an immune checkpoint inhibitor can be delivered by a virus provided herein.

There are a variety of immune checkpoints that can be targeted by an immune checkpoint modulator and/or any therapeutic protein, Table 2.

TABLE 2
Genes coding for immune checkpoints that are targeted by immune checkpoint modulators

| | | | NCBI number | |
|---|---|---|---|---|
| | | | (GRCh38.p2) | |
| Gene | | | *AC010327.8 | Location in |
| Symbol | Abbreviation | Name | **GRCh38.p7 | genome |
| ADORA | A2aR; RDC8; | adenosineA2a | 135 | 22q11.23 |
| 2A | ADORA2 | receptor | | |
| CD276 | B7H3; B7-H3; B7RP-2; | CD276 molecule | 80381 | 15q23-q24 |
| | 4Ig-B7-H3 | | | |
| VTCN1 | B7X; B7H4; B7S1; | V-set domain | 79679 | 1p13.1 |
| | B7-H4; B7h.5; | containing T cell | | |
| | VCTN1; PRO1291 | activation inhibitor 1 | | |
| BTLA | BTLA1; CD272 | B and T lymphocyte | 151888 | 3q13.2 |
| | | associated | | |
| CTLA4 | GSE; GRD4; ALPS5; | cytotoxic T-lymphocyte- | 1493 | 2q33 |
| | CD152; CTLA-4; | associated protein 4 | | |
| | IDDM12; CELIAC3 | | | |
| IDO1 | IDO; INDO; IDO-1 | indoleamine 2,3- | 3620 | 8p12-p11 |
| | | dioxygenase 1 | | |
| KIR3DL | KIR; NKB1; NKAT3; | killer cell | 3811 | 19q13.4 |
| 1 | NKB1B; NKAT-3; | immunoglobulin-like | | |
| | CD158E1; KIR3DL2; | receptor, three domains, | | |
| | KIR3DL1/S1 | long cytoplasmic tail, 1 | | |
| LAG3 | LAG3;CD223 | lymphocyte-activation | 3902 | 12p13.32 |
| | | gene 3 | | |
| PDCD1 | PD1; PD-1; CD279; | programmed cell | 5133 | 2q37.3 |
| | SLEB2; hPD-1; hPD-1; | death 1 | | |
| | hSLE1 | | | |
| HAVCR | TIM3; CD366; KIM-3; | hepatitis A virus | 84868 | 5q33.3 |
| 2 | TIMD3; Tim-3; TIMD-3; | cellular receptor 2 | | |
| | HAVcr-2 | | | |
| VISTA | C10orf54, differentiation | V-domain | 64115 | 10q22.1 |
| | of ESC-1 (Dies1); platelet | immunoglobulin | | |
| | receptor Gi24 precursor; | suppressor of T-cell | | |
| | PD1 homolog (PD1H) | activation | | |
| | B7H5; GI24; B7-H5; | | | |
| | SISP1; PP2135 | | | |
| CD244 | 2B4; 2B4; NAIL; Nmrk; | CD244 molecule, natural | 51744 | 1q23.3 |
| | NKR2B4; SLAMF4 | killer cell receptor 2B4 | | |
| CISH | CIS; G18; SOCS; CIS-1; | Cytokine Inducible | 1154 | 3p21.3 |
| | BACTS2 | SH2-Containing | | |
| | | protein | | |
| HPRT1 | HPRT; HGPRT | hypoxanthine | 3251 | Xq26.1 |
| | | phosphoribosyltransferase | | |
| | | 1 | | |
| AAV*S1 | AAV | adeno-associated virus | 14 | 19q13 |
| | | integration site 1 | | |
| CCR5 | CKR5; CCR-5; | chemokine (C-C motif) | 1234 | 3p21.31 |
| | CD195; CKR-5; | receptor 5 (gene/ | | |
| | CCCKR5; CMKBR5; | pseudogene) | | |
| | IDDM22; CC-CKR-5 | | | |
| CD160 | NK1; BY55 ; NK28 | CD160 molecule | 11126 | 1q21.1 |
| TIGIT | VSIG9; VSTM3; | T-cell immunoreceptor | 201633 | 3q13.31 |
| | WUCAM | with Ig and ITIM domains | | |
| CD96 | TACTILE | CD96 molecule | 10225 | 3q13.13- |
| | | | | q13.2 |
| CRTAM | CD355 | cytotoxic and regulatory | 56253 | 11q24.1 |
| | | T-cell molecule | | |
| LAIR1 | CD305; LAIR-1 | leukocyte associated | 3903 | 19q13.4 |
| | | immunoglobulin like | | |
| | | receptor 1 | | |
| SIGLEC 7 | p75; QA79; AIRM1; CD328; | sialic acid binding Ig | 27036 | 19q13.3 |
| | CDw328; D-siglec; | like lectin 7 | | |
| | SIGLEC-7; SIGLECP2; | | | |
| | SIGLEC19P; p75/AIRM1 | | | |
| SIGLEC 9 | CD329; CDw329; FOAP-9; | sialic acid binding Ig | 27180 | 19q13.41 |
| | siglec-9; OBBP-LIKE | like lectin 9 | | |
| TNFRSF | DR5; CD262; KILLER; | tumor necrosis factor | 8795 | 8p22-p21 |
| 10B | TRICK2; TRICKB; | receptor superfamily | | |
| | ZTNFR9; TRAILR2; | member 10b | | |
| | TRICK2A; TRICK2B; | | | |
| | TRAIL-R2; KILLER/DR5 | | | |
| TNFRSF | DR4; APO2; CD261; | tumor necrosis factor | 8797 | 8p21 |
| 10A | TRAILR1; TRAILR-1 | receptor superfamily | | |
| | | member 10a | | |
| CASP8 | CAP4; MACH; MCH5; | caspase 8 | 841 | 2q33-q34 |
| | FLICE; ALPS2B; Casp-8 | | | |
| CASP10 | MCH4; ALPS2; FLICE2 | caspase 10 | 843 | 2q33-q34 |
| CASP3 | CPP32; SCA-1; CPP32B | caspase 3 | 836 | 4q34 |
| CASP6 | MCH2 | caspase 6 | 839 | 4q25 |
| CASP7 | MCH3; CMH-1; LICE2; | caspase 7 | 840 | 10q25 |
| | CASP-7; ICE-LAP3 | | | |
| FADD | GIG3; MORT1 | Fas associated via | 8772 | 11q13.3 |
| | | death domain | | |
| FAS | APT1; CD95; FAS1; | Fas cell surface | 355 | 10q24.1 |
| | APO-1; FASTM; ALPS1A; | death receptor | | |
| | TNFRSF6 | | | |
| TGFBRII | AAT3; FAA3; LDS2; | transforming growth | 7048 | 3p22 |
| | MFS2; RIIC; LDS1B; | factor beta receptor | | |
| | LDS2B; TAAD2; | II | | |
| | TGFR-2; TGFbeta-RII | | | |
| TGFBR1 | AAT5; ALK5; ESS1; | transforming growth | 7046 | 9q22 |
| | LDS1; MSSE; SKR4; | factor beta receptor I | | |
| | ALK-5; LDS1A; | | | |
| | LDS2A; TGFR-1; | | | |
| | ACVRLK4; tbetaR-I | | | |
| SMAD2 | JV18; MADH2; | SMAD family | 4087 | 18q21.1 |
| | MADR2; JV18-1; | member 2 | | |
| | hMAD-2; hSMAD2 | | | |
| SMAD3 | LDS3; LDS1C; | SMAD family | 4088 | 15q22.33 |
| | MADH3; JV15-2; | member 3 | | |
| | HSPC193; HsT17436 | | | |
| SMAD4 | JIP; DPC4; MADH4; | SMAD family | 4089 | 18q21.1 |
| | MYHRS | member 4 | | |
| SKI | SGS; SKV | SKI proto-oncogene | 6497 | 1p36.33 |
| SKIL | SNO; SnoA; SnoI; SnoN | SKI-like proto-oncogene | 6498 | 3q26 |
| TGIF1 | HPE4; TGIF | TGFB induced factor | 7050 | 18p11.3 |
| | | homeobox 1 | | |
| IL10RA | CD210; IL10R; CD210a; | interleukin 10 receptor | 3587 | 11q23 |
| | CDW210A; HIL-10R; | subunit alpha | | |
| | IL-10R1 | | | |
| IL10RB | CRFB4; CRF2-4; D21S58; | interleukin 10 | 3588 | 21q22.11 |
| | D21S66; CDW210B; | receptor subunit beta | | |
| | IL-10R2 | | | |
| HMOX2 | HO-2 | heme oxygenase 2 | 3163 | 16p13.3 |
| IL6R | IL6Q; gp80; CD126; | interleukin 6 receptor | 3570 | 1q21 |
| | IL6RA; IL6RQ; IL-6RA; | | | |
| | IL-6R-1 | | | |
| IL6ST | CD130; GP130; CDW130; | interleukin 6 signal | 3572 | 5q11.2 |
| | IL-6RB | transducer | | |
| CSK | CSK | c-src tyrosine kinase | 1445 | 15q24.1 |
| PAG1 | CBP; PAG | phosphoprotein | 55824 | 8q21.13 |
| | | membrane anchor with | | |
| | | glycosphingolipid | | |
| | | microdomains 1 | | |
| SIT1 | SIT1 | signaling threshold | 27240 | 9p13-p12 |
| | | regulating transmembrane | | |
| | | adaptor 1 | | |
| FOXP3 | JM2; AIID; IPEX; PIDX; | forkhead box P3 | 50943 | Xp11.23 |
| | XPID; DIETER | | | |
| PRDM1 | BLIMP1; PRDI-BF1 | PR domain 1 | 639 | 6q21 |
| BATF | SFA2; B-ATF; BATF1; | basic leucine zipper | 10538 | 14q24.3 |
| | SFA-2 | transcription factor, | | |
| | | ATF-like | | |
| GUCY1 | GC-SA2; GUC1A2 | guanylate cyclase 1, | 2977 | 11q21-q22 |
| A2 | | soluble, alpha 2 | | |
| GUCY1 | GUCA3; MYMY6; | guanylate cyclase 1, | 2982 | 4q32.1 |
| A3 | GC-SA3; GUC1A3; | soluble, alpha 3 | | |
| | GUCSA3; GUCY1A1 | | | |
| GUCY1 | GUCY1B2 | guanylate cyclase 1, | 2974 | 13q14.3 |
| B2 | | soluble, beta 2 | | |
| | | (pseudogene) | | |
| GUCY1 | GUCB3; GC-SB3; | guanylate cyclase 1, | 2983 | 4q31.3-q33 |
| B3 | GUC1B3; GUCSB3; | soluble, beta 3 | | |
| | GUCY1B1; GC-S-beta-1 | | | |
| TRA | IMD7; TCRA; TCRD; | T-cell receptor alpha | 6955 | 14q11.2 |
| | TRAalpha; TRAC | locus | | |
| TRB | TCRB; TRBbeta | T cell receptor beta | 6957 | 7q34 |
| | | locus | | |
| EGLN1 | HPH2; PHD2; SM20; | egl-9 family hypoxia- | 54583 | 1q42.1 |
| | ECYT3; HALAH; HPH-2; | inducible factor 1 | | |
| | HIFPH2; ZMYND6; | | | |
| | Clorf12; HIF-PH2 | | | |
| EGLN2 | EIT6; PHD1; HPH-1; | egl-9 family hypoxia- | 112398 | 19q13.2 |
| | HPH-3; HIFPH1; HIF-PH1 | inducible factor 2 | | |
| EGLN3 | PHD3; HIFPH3; HIFP4H3 | egl-9 family hypoxia- | 112399 | 14q13.1 |
| | | inducible factor 3 | | |
| PPP1R12 | p84; p85; LENG3; MBS85 | protein phosphatase 1 | 54776 | 19q13.42 |
| C** | | regulatory subunit 12C | | |

The therapeutic protein, in some cases, can be a humanized anti-CD20 monoclonal antibody (e.g., Gazyva), a VEGFR Fc-fusion (e.g., Eylea), a CTLA-4 Fc-fusion (e.g., Nulojix), a glucagon-like peptide-1 receptor agonist Fc-fusion (e.g., Trulicity), VEGFR Fc-fusion (e.g., Zaltrap), a recombinant factor IX Fc fusion (e.g., Alprolix), a recombinant factor VIII Fc-fusion (e.g., Eloctate), a GLP-1 receptor agonist-albumin fusion (e.g., Tanzeum), a recombinant factor IX albumin fusion (e.g., Idelvion), a PEGylated IFNβ-1a (e.g., Plegridy), a recombinant factor VIII PEGylated (e.g., Adynovate), a humanized anti-HER2/neu conjugated to emtansine (e.g., Kadcyla), a mouse/human chimeric anti-CD30 (e.g., Adcetris), an anti-human epidermal growth factor receptor 2 (HER2) (e.g., Perjeta), Anti-IL-6 receptor (Actemra), an anti-CD20 (e.g., obinutuzumab; Gazyva), an anti-integrin a4b7 (LPAM-1) (e.g., Entyvio), an anti-PD-1 (e.g., Keytruda), an anti-dabigatran (e.g., Praxbind), an anti-IL-5 (e.g., Nucala), an Anti-CD319 (SLAMF7) (e.g., Empliciti), an anti-IL-17a (e.g., Taltz), an anti-IL-5 (e.g., Cinqair), an anti-PD-L1 (e.g., Tecentriq), an anti-CD25 (e.g., Zinbryta), an anti-CD30 (e.g., Adcetris), an anti-IL-6 (e.g., Sylvant), an anti-GD2 (e.g., Unituxin), an anti-Bacillus anthracis (e.g., Anthim), an anti-TNFα (e.g., Inflectra), a human anti-B-cell activating factor (BAFF) (e.g., belimumab), a human anti-CTLA-4 (e.g., ipilimumab), a CTLA-4 Fe-fusion (e.g., belatacept), humanized anti-human epidermal growth factor receptor 2 (HER2) (e.g., pertuzumab), a VEGFR Fc fusion (e.g., ziv-afilbercept), a G-CSF (e.g., tbo-filgrastim), human anti-VEGFR2 (KDR) (e.g., ramucirumab), a mouse/human chimeric anti-IL-6 (e.g., siltuximab), pembrolizumab, mouse bispecific anti-CD19/anti-CD3 (e.g., blintumomab), nivolumab, a parathyroid hormone, a mouse/human chimeric anti-GD2 (e.g., dinutuximab), a human anti-CD38 (e.g., daratumumab), a human anti-epidermal growth factor receptor (EGFR)(e.g., necitumumab), humanized anti-CD319 (SLAMF7) (e.g., elotuzumab), atezolizumab.

In an aspect, a therapeutic protein can be a cytokine. A cytokine can be a pro-inflammatory cytokine. In an aspect, a cytokine can be: IL-2, IL-7, IL-12, IL-15, IL-21, and combinations thereof. Additionally, pro-inflammatory cytokines can also be introduced with a virus provided herein. Non-limiting examples of pro-inflammatory cytokines include interleukin 6 (IL-6), interferon alpha (IFNα), interferon beta (IFNβ), C-C motif ligand 4 (CCL4), C-C motif ligand 5 (CCL5), C-X-C motif ligand 10 (CXCL10), interleukin 1 beta (IL-1β), IL-18 and IL-33.

In an aspect, a therapeutic protein can be a growth factor. In some aspects, a growth factor can be an interleukin. Non-limiting examples of interleukins include: interleukin-2 (IL-2), insulin, IFN-γ, IL-4, IL-7, GM-CSF, IL-10, IL-21, IL-15, TGF beta, and TNF alpha or any other additives for the growth of cells, such as immune cells.

In an aspect, a therapeutic protein can be a chemotherapeutic. A chemotherapeutic agent or compound can be a chemical compound useful in the treatment of cancer. The chemotherapeutic cancer agents that can be used in combination with the disclosed viruses include, but are not limited to, mitotic inhibitors (vinca alkaloids). These include vincristine, vinblastine, vindesine and Navelbine™ (vinorelbine, 5′-noranhydroblastine). In yet other cases, chemotherapeutic cancer agents include topoisomerase I inhibitors, such as camptothecin compounds. As used herein, “camptothecin compounds” include Camptosar™ (irinotecan HCL), Hycamtin™ (topotecan HCL) and other compounds derived from camptothecin and its analogues. Another category of chemotherapeutic cancer agents that can be used in the methods and compositions disclosed herein are podophyllotoxin derivatives, such as etoposide, teniposide and mitopodozide. The present disclosure further encompasses other chemotherapeutic cancer agents known as alkylating agents, which alkylate the genetic material in tumor cells. These include without limitation cisplatin, cyclophosphamide, nitrogen mustard, trimethylene thiophosphoramide, carmustine, busulfan, chlorambucil, belustine, uracil mustard, chlomaphazin, and dacarbazine. The disclosure encompasses antimetabolites as chemotherapeutic agents. Examples of these types of agents include cytosine arabinoside, fluorouracil, methotrexate, mercaptopurine, azathioprime, and procarbazine.

In an aspect, a therapeutic agent can be an antibiotic. In some cases, an antibiotic can be a bacterial wall targeting agent, a cell membrane targeting agent, a bacterial enzyme interfering agent, a bactericidal agent, a protein synthesis inhibitor, or a bacteriostatic agent. A bacterial wall targeting agent can be a penicillin derivatives (penams), cephalosporins (cephems), monobactams, and carbapenems. β-Lactam antibiotics are bactericidal or bacteriostatic and act by inhibiting the synthesis of the peptidoglycan layer of bacterial cell walls. In some cases, an antibiotic may be a protein synthesis inhibitor. A protein synthesis inhibitor can be ampicillin which acts as an irreversible inhibitor of the enzyme transpeptidase, which is needed by bacteria to make the cell wall. It inhibits the third and final stage of bacterial cell wall synthesis in binary fission, which ultimately leads to cell lysis; therefore, ampicillin is usually bacteriolytic. In some cases, a bactericidal agent can be cephalosporin or quinolone. In other cases, a bacteriostatic agent is trimethoprim, sulfamethoxazole, or pentamidine. Additional examples of antibiotics include without limitation doxorubicin, bleomycin, dactinomycin, daunorubicin, mithramycin, mitomycin, mytomycin C, and daunomycin.

In an aspect, a therapeutic agent can also be an antibody or portion thereof. An antibody can be a polyclonal antibody or a monoclonal antibody. A polyclonal antibody that can be administered can be an antilymphocyte or antithymocyte antigen. A monoclonal antibody can be an anti-IL-2 receptor antibody, an anti-CD25 antibody, or an anti-CD3 antibody. An anti-CD20 antibody can also be used. B-cell ablative therapy such as agents that react with CD20, e.g., Rituxan can also be used as immunosuppressive agents. Exemplary anti-cancer antibodies include but are not limited to: dacarbazine, azacytidine, amsacrine, melphalan, ifosfamide and mitoxantrone.

In an aspect, a therapeutic agent can also be an anti-fungal. In some cases, an antifungal agent can be from a class of polyene, azole, allylamine, or echinocandin. In some embodiments, a polyene antifungal is amphotericin B, candicidin, filipin, hamycin, natamycin, nystatin, or rimocidin. In some cases, an antifungal can be from an azole family. Azole antifungals can inhibit lanosterol 14 α-demethylase. An azole antifungal can be an imidazole such as bifonazole, butoconazole, clotrimazole, econazole, fenticonazole, isoconazole, ketoconazole, luliconazole, miconazole, omoconazole, oxiconazole, sertaconazole, sulcoazole, or tioconazole. An azole antifungal can be a triazole such as albaconazole, efinaconazole, epoxiconazole, fluconazole, isavuvonazole, itraconazole, posaconazole, propiconazole, ravuconazole, terconazole, or voriconazole. In some cases, an azole can be a thiazole such as abafungin. An antifungal can be an allylamine such as amorolfin, butenafine, naftifine, or terbinafine. An antifungal can also be an echinocandin such as anidulafungin, caspofungin, or micafungin. Additional agents that can be antifungals can be aurones, benzoic acid, ciclopirox, flucytosine, griseofulvin, haloprogin, tolnaftate, undecylenic acid, crystal violet or balsam of Peru.

### Pharmaceutical Compositions

Provided herein is a pharmaceutically acceptable viral therapeutic. In an aspect, provided herein are also methods of implementing pharmaceutically acceptable viral therapeutics comprising oncolytic viruses. Compositions and methods provided herein can be used for the treatment of a disease, such as a cancer.

Cancer can include, but is not limited to, melanoma, hepatocellular carcinoma, breast cancer, lung cancer, peritoneal cancer, prostate cancer, bladder cancer, ovarian cancer, leukemia, lymphoma, renal carcinoma, pancreatic cancer, epithelial carcinoma, gastric cancer, colon carcinoma, duodenal cancer, pancreatic adenocarcinoma, mesothelioma, glioblastoma multiforme, astrocytoma, multiple myeloma, prostate carcinoma, hepatocellular carcinoma, cholangiosarcoma, pancreatic adenocarcinoma, head and neck squamous cell carcinoma, colorectal cancer, intestinal-type gastric adenocarcinoma, cervical squamous-cell carcinoma, osteosarcoma, epithelial ovarian carcinoma, acute lymphoblastic lymphoma, myeloproliferative neoplasms, and sarcoma.

In an aspect, an oncolytic virus can amplify a therapeutic agent selectively within a cancer. In an aspect, an oncolytic virus can also carry a therapeutic protein or a diagnostic protein and express the protein or a functional fragment thereof in proximity to a cancer. In an aspect, a therapeutic protein or a diagnostic protein can enhance an endogenous immune response. In an aspect, an oncolytic virus and/or a retrovirus provided herein coupled with a therapeutic protein provided herein can have greater anti-cancer effects as compared to a comparable virus absent the coupling with the therapeutic agent. In an aspect, the oncolytic virus and/or a retrovirus provided herein coupled with a therapeutic protein provided herein can have from about 1 fold, 2 fold, 3 fold, 4 fold, 5 fold, 6 fold, 7 fold, 8 fold, 9 fold, or up to about 10 fold greater anti-cancer effects as compared to a comparable a virus absent the coupling with the therapeutic agent. In an aspect, the oncolytic virus and/or a retrovirus provided herein coupled with a therapeutic protein provided herein can reduce a cancer by about 10%, 20%, 30%, 40%, 50%, 60%, 70%, 80%, 90%, or up to 100% more as compared to a comparable virus absent the coupling with the therapeutic agent.

In an aspect, provided herein can also be methods of reducing toxicity of cells contacted with nucleic acids, polypeptides, vectors, and plasmids provided herein. For example, a vector encoding for a virus provided herein can be polycistronic, thereby introducing multiple elements, such as a virus, in a single vector construct and reducing toxicity associated with a vector. In an aspect, a bicistronic vector coding for an oncolytic virus provided herein and a therapeutic protein provided herein can have from about 1 fold, 2 fold, 3 fold, 4 fold, 5 fold, 6 fold, 7 fold, 8 fold, 9 fold, or up to about 10 fold less toxicity as compared to introducing two monocistronic vectors the first one coding for the oncolytic virus provided herein and the second one coding for the therapeutic agent. In some cases, cell toxicity is reduced by about, at least about, or at most about 1%, 2%, 3%, 4%, 5%, 6%, 7%, 8%, 9%, 10%, 12%, 15%, 17%, 18%, 19%, 20%, 25%, 30%, 35%, 40%, 45%, 50%, 55%, 60%, 65%, 70%, 75%, 80%, 82%, 85%, 88%, 90%, 92%, 95%, 97%, 98%, 99% or 100% when at least a bicistronic vector provided herein is used as compared to introducing each element alone in a dual vector system.

As described herein, viral particles provided herein, can be used to deliver a viral vector comprising a transgene comprising at least a portion of a replication defective virus into a cell ex vivo or in vivo. In some embodiments, a virus provided herein can be measured as pfu (plaque forming units). In some cases, the pfu of a viral dosage of the compositions and methods of the disclosure can be about 10⁸ to about 5×10¹⁰ pfu. In some cases, a dosage of a virus provided herein is at least about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, and 5×10¹⁰ pfu. In some cases, a viral of this disclosure is at most about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵ pfu. In some aspects, a viral vector of the disclosure can be measured as vector genomes. In some cases, viruses of this disclosure are 1×10¹⁰ to 3×10¹² vector genomes, or 1×10⁹ to 3×10¹³ vector genomes, or 1×10⁸ to 3×10¹⁴ vector genomes, or at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ vector genomes, or are 1×10⁸ to 3×10¹⁴ vector genomes, or are at most about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ vector genomes.

In some cases, the dosage of a virus or a viral vector of the disclosure can be measured using multiplicity of infection (MOI). In some cases, MOI can refer to the ratio, or multiple of vector or viral genomes to the cells to which the nucleic can be delivered. In some cases, the MOI can be 1×10⁶ GC/mL (genome copies/mL). In some cases, the MOI can be 1×10⁵ GC/mL to 1×10⁷ GC/mL. In some cases, the MOI can be 1×10⁴ GC/mL to 1×10⁸ GC/mL. In some cases, recombinant viruses of the disclosure are at least about 1×10¹ GC/mL, 1×10² GC/mL, 1×10³ GC/mL, 1×10⁴ GC/mL, 1×10⁵ GC/mL, 1×10⁶ GC/mL, 1×10⁷ GC/mL, 1×10⁸ GC/mL, 1×10⁹ GC/mL, 1×10¹⁰ GC/mL, 1×10¹¹ GC/mL, 1×10¹² GC/mL, 1×10¹³ GC/mL, 1×10¹⁴ GC/mL, 1×10¹⁵ GC/mL, 1×10¹⁶ GC/mL, 1×10¹⁷ GC/mL, and 1×10¹⁸ GC/mL MOI. In some cases, a virus of this disclosure are from about 1×10⁸ GC/mL to about 3×10¹⁴ GC/mL MOI, or are at most about 1×10¹ GC/mL, 1×10² GC/mL, 1×10³ GC/mL, 1×10⁴ GC/mL, 1×10⁵ GC/mL, 1×10⁶ GC/mL, 1×10⁷ GC/mL, 1×10⁸ GC/mL, 1×10⁹ GC/mL, 1×10¹⁰ GC/mL, 1×10¹¹ GC/mL, 1×10¹² GC/mL, 1×10¹³ GC/mL, 1×10¹⁴ GC/mL, 1×10¹⁵ GC/mL, 1×10¹⁶ GC/mL, 1×10¹⁷ GC/mL, and 1×10¹⁸ GC/mL MOI.

Vectors described herein, can be delivered by any suitable method, including transfection, electroporation, liposome delivery, membrane fusion techniques, high velocity DNA-coated pellets, viral infection and protoplast fusion. Electroporation using, for example, the Neon® Transfection System (ThermoFisher Scientific) or the AMAXA® Nucleofector (AMARA® Biosystems) can also be used for delivery of nucleic acids into a cell.

The transfection efficiency of cells, such as producer cells, with any of the vectors and plasmids provided herein can be or can be about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9%. In some embodiments, the transfection efficiency can be or can be about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9% than the transfection efficiency of comparable cells using a control delivery platform (e.g., a virus engineered absent the methods provided herein). In some cases, transfection or transduction efficiency can be quantified absent a cellular selection, sorting, or the like.

In an aspect, a vector provided herein can be delivered in vivo by administration to an individual subject, typically by systemic administration (e.g., intravenous, intraperitoneal, intramuscular, subdermal, or intracranial infusion).

### Kits

Disclosed herein can be kits comprising viral compositions. Disclosed herein can also be kits for the treatment or prevention of a cancer, pathogen infection, and/or immune disorder. In one embodiment, a kit can include a therapeutic or prophylactic composition containing an effective amount of a viral composition in unit dosage form. In some embodiments, a kit comprises a sterile container which can contain a therapeutic composition of virus; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In some cases, a virus provided herein, can be provided together with instructions for administering the virus to a subject having or at risk of developing a cancer, pathogen infection, immune disorder or that will undergo transplant. Instructions can generally include information about the use of the composition for the treatment or prevention of cancer, pathogen infection, immune disorder or transplant.

### EXAMPLES

The examples below further illustrate the described embodiments without limiting the scope of this disclosure.

### Example 1: Vector Design and Sequences of a Viral Construct Comprising a Non-Replicating Lentivirus

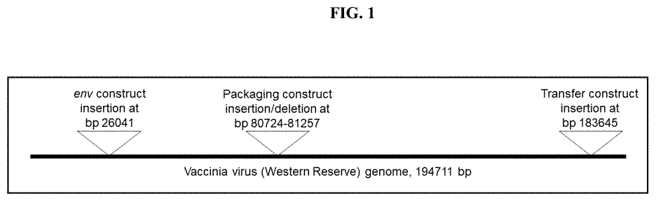

A viral vector comprising a non-replicating lentivirus was generated as follows. The env, packaging, and transfer elements were integrated into the wild type vaccinia virus (VV) genome, vaccinia virus Western Reserve strain with GenBank accession number AY243312.1 (Table 1 and SEQ ID NO: 1), separated by tens of thousands of bases all, as shown in FIG. 1. The viral vector contains a hybrid 5′ LTR region that is a fusion between a vaccinia virus promoter and an HIV-1 5′ LTR that functions absent TAT for transcription by utilizing vaccinia virus transcription factors. Vaccinia virus strong early promoters were added for each component and a strong early promoter was fused to the 5′ LTR of the transfer construct. The termination of vaccinia virus early gene transcription utilizes the termination sequence UUUUUNU (USNU) (SEQ ID NO: 7) to attract vaccinia virus-specific factors to halt transcription and generate poly(A) tails to protect viral mRNA. The sequence ATTTTTAT (SEQ ID NO: 8) was added to the desired terminal regions of each construct.

### Env Construct

The vsvg-containing env construct was designed to recombine with the vaccinia virus genome at a point between the vacwr032 (k11) and vacwr033 (k21) genes. A strong vaccinia virus early promoter was used:

(SEQ ID NO: 9)
TATTTATATTCCAAAAAAAAAAAATAAAATTTCAATTTTT

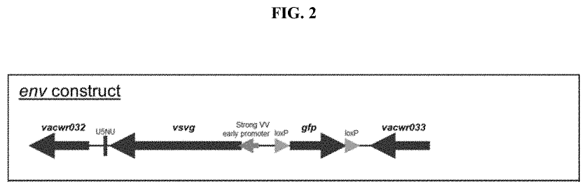

A map and sequence with elements marked in similar colors as the map is shown below in FIG. 2 and Table 2.

TABLE 2
Map of a lentiviral env construct: Vacwr032 (1-790); U5NU (906-914 and
917-925); VSVG (926-2461); vaccinia virus promoter (2470-2509); loxP
(2529-2562 and 3343-3376); GFP (2606-3322); Vacwr033 (3503-4416)

| SEQ | | |
|---|---|---|
| ID | ID | Sequence |
| 2 | Lenti | CACAATTGACGTACATGAGTCTGAGTTCCTTGTTTTTGCTAATTATTTCATCCAATTT |
| | viral | ATTATTCTTGACTATATCGAGATCTTTTGTATAGGAGTCAGACTTGTATTCAACATGC |
| | env | TTTTCTATAATCATTTTAGCTATTTCGGCATCATCCAATAGTACATTTTCCAGATTAG |
| | construct | CAGAATAGATATTAATGTCGTATTTGAACAGAGCCTGTAACATCTCAATGTCTTTATT |
| | | ATCTATAGCCAATTTAATGTCCGGAATGAAGAGAAGGGAATTATTGGTGTTTGTCGAC |
| | | GTCATATAGTCGAGCAAGAGAATCATCATATCCACGTGTCCATTTTTTATAGTGATGT |
| | | GAATACAACTAAGGAGAATAGCCAGATCAAAAGTAGATGGTATCTCTGAAAGAAAGTA |
| | | GGAAACAATACTTACATCATTAAGCATGACGGCATGATAAAATGAAGTTTTCCATCCA |
| | | GTTTTCCCATAGAACATCAGTCTCCAATTTTTCTTAACAAACAGTTTTACCGTTTGCA |
| | | TGTTACCACTATCAACCGCATAATACAATGCGGTGTTTCCCTTGTCATCAAATTGTGA |
| | | ATCATCCAGTCCACTGAATAGCAAAATCTTTACTATTTTGGTATCTTCCAATGTGGCT |
| | | GCCTGATGTAATGGAAATTCATTCTCTAGAAGATTTTTCAATGCTCCAGCGTTCAACA |
| | | ACGTACATACTAGACGCACGTTATTATCAGCTATTGCATAATACAAGGCACTATGTCC |
| | | ATGGACATCCGCCTTAAATGCATCTTTGCTAGAGAGGTCTGAAACGAGACGCTAATTA |
| | | GTGTATATTTTTTCATTTTTTATAATTTTGTCATATTGCACCAGAATTAATAATATCT |
| | | CTAATAGATCTGATTAGTAGATACATGGCTATCGCATAAAAATTGCATAAAAATTTTA |
| | | CTTTCCAAGTCGGTTCATCTCTATGTCTGTATAAATCTGTCTTTTCTTGGTGTGCTTT |
| | | AATTTAATGCAAAGATGGATACCAACTCGGAGAACCAAGAATAGTCCAATGATTAACC |
| | | CTATGATAAAGAAGAAAGAGGCAATAGAGCTTTTCCAACTACTGAACCAACCTTCTAC |
| | | AAGCTCGATTGGATTTTTGGATAGCCCAGTATCACCAAAGAATAAACTCTCATCATCA |
| | | GGAAGTTGCGAAGCAGCGTCTTGAATGTGAGGATGTTCGAACACCTGAGCCTTTGAGC |
| | | TAAGATGAAGATCGGAGTCCAACATACCATGTCCAATCATGTATAAAGGAAACTTATA |
| | | TCCTGAACTGGTCCTCAGAACTCCATTGGGTCCAATTTCCACGTCTTCATATGGTGCC |
| | | CAGTCATCCCACAGTTCCCTTTCTGTGGTAGTTCCACTGATCATTCCGACCATTCTTG |
| | | AGAGGATTGGAGCAGCAATATCGACTCTGATGTATCTGGTCTCAAAGTATTTTAGGGT |
| | | ACCATTGATTATGGTGAAAGCAGGACCGGTTCCTGGGTTTTTAGGAGCAAGATAGCTG |
| | | AGATCCACTGGAGAGATTGGAAGACCCGCTCTGATTTTGCTCCAGGTTTCTTGGCAGA |
| | | GGGAATAATCCAAGATCCTCTCAACGTCCTGAATTAGACTTACATCCACTGAGGTCTG |
| | | AGATGGAGCAGAGATACTTGACCCTTCTGGGCATTCAGGGAATCTGGCTGCAGCAAAG |
| | | AGATCCTTATCAGCCATCTCGAACCAGACACCTGATGGGAGTCTGACTCCCCAATGCT |
| | | TGCAGTATTGCATTTTGCAGGCCTTGCCTCCAGTTTCATAAGCAAAGTAGTTACTTCT |
| | | GAACCCTGTGCCCTCCTTTCCCAGGGATGATAGCTCTCCGTCCTCTGAGAAGAAGGTG |
| | | ATGTCCATGGAAATGAGGTTAGAATCACATAGCCCTTTGACCTTATAGTCAGAATGCC |
| | | AGGTTGTAGAGTTATGGACAGTGGGGCATATGTAATTGCTGCATTTTCCGTTGATGAA |
| | | CTGTGAATCAACCCATTCTCCTGTGTATTCATCAACCAGCACATGGTGAGGAGTCACC |
| | | TGGACAATCACTGCTTCGGCATCCGTCACAGTTGCATATCCACAACTTTGAGGAGGGA |
| | | AGCCTGGATTCAGCCAAGTTCCTTGTTTCGTTTGTTCAATGCTTTCCTTGCATTGTTC |
| | | TACAGATGGAGTGAAGGATCGGATGGACTGTGTTATATACTTCGGTCCATACCAGCGG |
| | | AAATCACAAGTAGTGACCCATTTGGAAGCATGACACATCCAACCGTCTGCTTGAATAG |
| | | CCTTGTGACTCTTGGGCATTTTGACTTGTATGGCTGTGCCTATTAAGTCATTATGCCA |
| | | ATTTAAATCTGAGCTTGACGGGCAATAATGGTAATTAGAAGGAACATTTTTCCAGTTT |
| | | CCTTTTTGGTTGTGTGGGAAAACTATGGTGAACTTGCAATTCACCCCAATGAATAAGA |
| | | AGGCTAAGTACAAAAGGCACTTCATTAGAACGTTATTTATATTCCAAAAAAAAAAAAT |
| | | AAAATTTCAATTTTTGCTAGCGGGCCCTAAGGCCATAACTTCGTATAGCATACATTAT |
| | | ACGAAGTTATAATTCTGCAGATGAATTTCATTTTGTTTTTTTCTATGCTATAAATGCG |
| | | TAAAGGCGAAGAGCTGTTCACTGGTGTCGTCCCTATTCTGGTGGAACTGGATGGTGAT |
| | | GTCAACGGTCATAAGTTTTCCGTGCGTGGCGAGGGTGAAGGTGACGCAACTAATGGTA |
| | | AACTGACGCTGAAGTTCATCTGTACTACTGGTAAACTGCCGGTACCTTGGCCGACTCT |
| | | GGTAACGACGCTGACTTATGGTGTTCAGTGCTTTGCTCGTTATCCGGACCATATGAAG |
| | | CAGCATGACTTCTTCAAGTCCGCCATGCCGGAAGGCTATGTGCAGGAACGCACGATTT |
| | | CCTTTAAGGATGACGGCACGTACAAAACGCGTGCGGAAGTGAAATTTGAAGGCGATAC |
| | | CCTGGTAAACCGCATTGAGCTGAAAGGCATTGACTTTAAAGAAGACGGCAATATCCTG |
| | | GGCCATAAGCTGGAATACAATTTTAACAGCCACAATGTTTACATCACCGCCGATAAAC |
| | | AAAAAAATGGCATTAAAGCGAATTTTAAAATTCGCCACAACGTGGAGGATGGCAGCGT |
| | | GCAGCTGGCTGATCACTACCAGCAAAACACTCCAATCGGTGATGGTCCTGTTCTGCTG |
| | | CCAGACAATCACTATCTGAGCACGCAAAGCGTTCTGTCTAAAGATCCGAACGAGAAAC |
| | | GCGATCATATGGTTCTGCTGGAGTTCGTAACCGCAGCGGGCATCACGCATGGTATGGA |
| | | TGAACTGTACAAATAATTAATTAAAAGGCCGCTCGAATAACTTCGTATAGCATACATT |
| | | ATACGAAGTTATGCTAGCACGATGCTCTTAGAATAACAACATATACACATTTAATAAA |
| | | AATAATATTTATTAAGAAAATTCAGATTTCACGTACCCATCAATATAAATAAAATAAT |
| | | GATTCCTTACACCGTACCCATATTAAGGAGATTCCACCTTACCCATAAACAATATAAA |
| | | TCCAGTAATATCATGTCTGATGATGAACACAAATGGTGTATTAAATTCCAGTTTTTCA |
| | | GGAGATGATCTCGCCGTAGCTACCATAATAGTAGATGCCTCTGCTACAGTTCCTTGTT |
| | | CGTCGACATCTATCTTTGCATTCTGAAACATTTTATAAATATATAATGGGTCCCTAGT |
| | | CATATGTTTAAACGACGCATTATCTGGATTAAACATACTAGGAGCCATCATTTCGGCT |
| | | ATCGACTTAATATCCCTCTTATTTTCGATAGAAAATTTAGGGAGTTTAAGATTGTACA |
| | | CTTTATTCCCTAATTGAAACGACCAATAGTCTAATTTTGCAGCCGTAATAGAATCTGT |
| | | GAAATGGGTCATATTATCACCTATTGCCAGGTACATACTAATATTAGCATCCTTATAC |
| | | GGAAGGCGTACCATATCATATTCTTCGTCATCGATTGTGATTGTATTTCCTTGCAATT |
| | | TAGTAACTACGTTCATCATGGGAACCGTTTTCGTACCGTACTTATTAGTAAAACTAGC |
| | | ATTGCGTGTTTTAGTGATATCAAACGGATATTGCCATATACCTTTAAAATATATAGTA |
| | | TTAATGATTGCCCATAGAGTATTATTGTCGAGCATATTAGAATCTACTACATTAGACA |
| | | TACCGGATCTACGTTCTACTATAGAATTAATTTTATTAACCGCATCTCGTCTAAAGTT |
| | | TAATCTATATAGGCCGAATCTATGATATTGTTGATAATACGACGGTTTAATGCACACA |
| | | GTATTATCTACGAAACTTTGATAAGTTAGATCAGTGTACGTATATTTAGATGTTTTCA |
| | | GCTTAGCTAATCCTGATATTAATTCTGTAAATGCTGGACCCAGATCTCTTTTTCTCAA |
| | | ATCCATAG |
| 3 | vsvg | TTACTTTCCAAGTCGGTTCATCTCTATGTCTGTATAAATCTGTCTTTTCTTGGTGTGC |
| | | TTTAATTTAATGCAAAGATGGATACCAACTCGGAGAACCAAGAATAGTCCAATGATTA |
| | | ACCCTATGATAAAGAAGAAAGAGGCAATAGAGCTTTTCCAACTACTGAACCAACCTTC |
| | | TACAAGCTCGATTGGATTTTTGGATAGCCCAGTATCACCAAAGAATAAACTCTCATCA |
| | | TCAGGAAGTTGCGAAGCAGCGTCTTGAATGTGAGGATGTTCGAACACCTGAGCCTTTG |
| | | AGCTAAGATGAAGATCGGAGTCCAACATACCATGTCCAATCATGTATAAAGGAAACTT |
| | | ATATCCTGAACTGGTCCTCAGAACTCCATTGGGTCCAATTTCCACGTCTTCATATGGT |
| | | GCCCAGTCATCCCACAGTTCCCTTTCTGTGGTAGTTCCACTGATCATTCCGACCATTC |
| | | TTGAGAGGATTGGAGCAGCAATATCGACTCTGATGTATCTGGTCTCAAAGTATTTTAG |
| | | GGTACCATTGATTATGGTGAAAGCAGGACCGGTTCCTGGGTTTTTAGGAGCAAGATAG |
| | | CTGAGATCCACTGGAGAGATTGGAAGACCCGCTCTGATTTTGCTCCAGGTTTCTTGGC |
| | | AGAGGGAATAATCCAAGATCCTCTCAACGTCCTGAATTAGACTTACATCCACTGAGGT |
| | | CTGAGATGGAGCAGAGATACTTGACCCTTCTGGGCATTCAGGGAATCTGGCTGCAGCA |
| | | AAGAGATCCTTATCAGCCATCTCGAACCAGACACCTGATGGGAGTCTGACTCCCCAAT |
| | | GCTTGCAGTATTGCATTTTGCAGGCCTTGCCTCCAGTTTCATAAGCAAAGTAGTTACT |
| | | TCTGAACCCTGTGCCCTCCTTTCCCAGGGATGATAGCTCTCCGTCCTCTGAGAAGAAG |
| | | GTGATGTCCATGGAAATGAGGTTAGAATCACATAGCCCTTTGACCTTATAGTCAGAAT |
| | | GCCAGGTTGTAGAGTTATGGACAGTGGGGCATATGTAATTGCTGCATTTTCCGTTGAT |
| | | GAACTGTGAATCAACCCATTCTCCTGTGTATTCATCAACCAGCACATGGTGAGGAGTC |
| | | ACCTGGACAATCACTGCTTCGGCATCCGTCACAGTTGCATATCCACAACTTTGAGGAG |
| | | GGAAGCCTGGATTCAGCCAAGTTCCTTGTTTCGTTTGTTCAATGCTTTCCTTGCATTG |
| | | TTCTACAGATGGAGTGAAGGATCGGATGGACTGTGTTATATACTTCGGTCCATACCAG |
| | | CGGAAATCACAAGTAGTGACCCATTTGGAAGCATGACACATCCAACCGTCTGCTTGAA |
| | | TAGCCTTGTGACTCTTGGGCATTTTGACTTGTATGGCTGTGCCTATTAAGTCATTATG |
| | | CCAATTTAAATCTGAGCTTGACGGGCAATAATGGTAATTAGAAGGAACATTTTTCCAG |
| | | TTTCCTTTTTGGTTGTGTGGGAAAACTATGGTGAACTTGCAATTCACCCCAATGAATA |
| | | AGAAGGCTAAGTACAAAAGGCACTTCAT |

### Packaging Construct

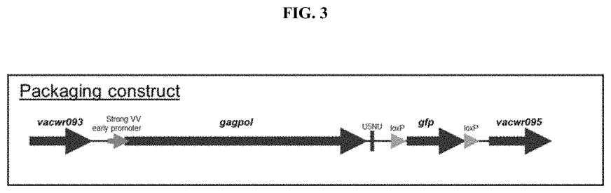

The packaging construct was designed to recombine with the vaccinia virus genome to replace the coding sequence for vacwr094 (j2r) between the vacwr093 (fir) and vacwr033 (j3r) genes. This deletion was chosen because j2r-deletion creates an attenuated virus that preferentially infects tumor cells. Also, this insertion positions the construct near the center of the vaccinia virus genome which is relatively distant from the other constructs. An annotated map and sequence with elements marked in similar colors as the map is shown in FIG. 3 and

TABLE 3
The packaging construct utilizes a strong vaccinia virus early promoter:
AAAAATTGAAATTTTATTTTTTTTTTTTGGAATATAAATA(SEQ ID NO: 10)
Table 3: Map of the lentiviral packaging construct: vacwr093 (524-985);
Vaccinia virus promoter (1000-1039); gagpol (1040-5346); U5NU (5750-5757
and 5773-5780); loxP (5869-5902 and 6699-6732); GFP (5979-6698); vacwr095
(6798-7729)

| SEQ | | |
|---|---|---|
| ID | ID | Sequence |
| 4 | Lenti | CACAATTGACGTACATGAGTCTGAGTTCCTTGTTTTTGCTAATTATTTCATCCAATTTAT |
| | viral | TATTCTTGACTATATCGAGATCTTTTGTATAGGAGTCAGACTTGTATTCAACATGCTTTT |
| | packaging | CTATAATCATTTTAGCTATTTCGGCATCATCCAATAGTACATTTTCCAGATTAGCAGAAT |
| | construct | AGATATTAATGTCGTATTTGAACAGAGCCTGTAACATCTCAATGTCTTTATTATCTATAG |
| | | CCAATTTAATGTCCGGAATGAAGAGAAGGGAATTATTGGTGTTTGTCGACGTCATATAGT |
| | | CGAGCAAGAGAATCATCATATCCACGTGTCCATTTTTTATAGTGATGTGAATACAACTAA |
| | | GGAGAATAGCCAGATCAAAAGTAGATGGTATCTCTGAAAGAAAGTAGGAAACAATACTTA |
| | | CATCATTAAGCATGACGGCATGATAAAATGAAGTTTTCCATCCAGTTTTCCCATAGAACA |
| | | TCAGTCTCCAATTTTTCTTAACAAACAGTTTTACCGTTTGCATGTTACCACTATCAACCG |
| | | CATAATACAATGCGGTGTTTCCCTTGTCATCAAATTGTGAATCATCCAGTCCACTGAATA |
| | | GCAAAATCTTTACTATTTTGGTATCTTCCAATGTGGCTGCCTGATGTAATGGAAATTCAT |
| | | TCTCTAGAAGATTTTTCAATGCTCCAGCGTTCAACAACGTACATACTAGACGCACGTTAT |
| | | TATCAGCTATTGCATAATACAAGGCACTATGTCCATGGACATCCGCCTTAAATGCATCTT |
| | | TGCTAGAGAGGTCTGAAACGAGACGCTAATTAGTGTATATTTTTTCATTTTTTATAATTT |
| | | TGTCATATTGCACCAGAATTAATAATATCTCTAATAGATCTGATTAGTAGATACATGGCT |
| | | ATCGCATAAAAATTGCATAAAAATTTTACTTTCCAAGTCGGTTCATCTCTATGTCTGTAT |
| | | AAATCTGTCTTTTCTTGGTGTGCTTTAATTTAATGCAAAGATGGATACCAACTCGGAGAA |
| | | CCAAGAATAGTCCAATGATTAACCCTATGATAAAGAAGAAAGAGGCAATAGAGCTTTTCC |
| | | AACTACTGAACCAACCTTCTACAAGCTCGATTGGATTTTTGGATAGCCCAGTATCACCAA |
| | | AGAATAAACTCTCATCATCAGGAAGTTGCGAAGCAGCGTCTTGAATGTGAGGATGTTCGA |
| | | ACACCTGAGCCTTTGAGCTAAGATGAAGATCGGAGTCCAACATACCATGTCCAATCATGT |
| | | ATAAAGGAAACTTATATCCTGAACTGGTCCTCAGAACTCCATTGGGTCCAATTTCCACGT |
| | | CTTCATATGGTGCCCAGTCATCCCACAGTTCCCTTTCTGTGGTAGTTCCACTGATCATTC |
| | | CGACCATTCTTGAGAGGATTGGAGCAGCAATATCGACTCTGATGTATCTGGTCTCAAAGT |
| | | ATTTTAGGGTACCATTGATTATGGTGAAAGCAGGACCGGTTCCTGGGTTTTTAGGAGCAA |
| | | GATAGCTGAGATCCACTGGAGAGATTGGAAGACCCGCTCTGATTTTGCTCCAGGTTTCTT |
| | | GGCAGAGGGAATAATCCAAGATCCTCTCAACGTCCTGAATTAGACTTACATCCACTGAGG |
| | | TCTGAGATGGAGCAGAGATACTTGACCCTTCTGGGCATTCAGGGAATCTGGCTGCAGCAA |
| | | AGAGATCCTTATCAGCCATCTCGAACCAGACACCTGATGGGAGTCTGACTCCCCAATGCT |
| | | TGCAGTATTGCATTTTGCAGGCCTTGCCTCCAGTTTCATAAGCAAAGTAGTTACTTCTGA |
| | | ACCCTGTGCCCTCCTTTCCCAGGGATGATAGCTCTCCGTCCTCTGAGAAGAAGGTGATGT |
| | | CCATGGAAATGAGGTTAGAATCACATAGCCCTTTGACCTTATAGTCAGAATGCCAGGTTG |
| | | TAGAGTTATGGACAGTGGGGCATATGTAATTGCTGCATTTTCCGTTGATGAACTGTGAAT |
| | | CAACCCATTCTCCTGTGTATTCATCAACCAGCACATGGTGAGGAGTCACCTGGACAATCA |
| | | CTGCTTCGGCATCCGTCACAGTTGCATATCCACAACTTTGAGGAGGGAAGCCTGGATTCA |
| | | GCCAAGTTCCTTGTTTCGTTTGTTCAATGCTTTCCTTGCATTGTTCTACAGATGGAGTGA |
| | | AGGATCGGATGGACTGTGTTATATACTTCGGTCCATACCAGCGGAAATCACAAGTAGTGA |
| | | CCCATTTGGAAGCATGACACATCCAACCGTCTGCTTGAATAGCCTTGTGACTCTTGGGCA |
| | | TTTTGACTTGTATGGCTGTGCCTATTAAGTCATTATGCCAATTTAAATCTGAGCTTGACG |
| | | GGCAATAATGGTAATTAGAAGGAACATTTTTCCAGTTTCCTTTTTGGTTGTGTGGGAAAA |
| | | CTATGGTGAACTTGCAATTCACCCCAATGAATAAGAAGGCTAAGTACAAAAGGCACTTCA |
| | | TTAGAACGTTATTTATATTCCAAAAAAAAAAAATAAAATTTCAATTTTTGCTAGCGGGCC |
| | | CTAAGGCCATAACTTCGTATAGCATACATTATACGAAGTTATAATTCTGCAGATGAATTT |
| | | CATTTTGTTTTTTTCTATGCTATAAATGCGTAAAGGCGAAGAGCTGTTCACTGGTGTCGT |
| | | CCCTATTCTGGTGGAACTGGATGGTGATGTCAACGGTCATAAGTTTTCCGTGCGTGGCGA |
| | | GGGTGAAGGTGACGCAACTAATGGTAAACTGACGCTGAAGTTCATCTGTACTACTGGTAA |
| | | ACTGCCGGTACCTTGGCCGACTCTGGTAACGACGCTGACTTATGGTGTTCAGTGCTTTGC |
| | | TCGTTATCCGGACCATATGAAGCAGCATGACTTCTTCAAGTCCGCCATGCCGGAAGGCTA |
| | | TGTGCAGGAACGCACGATTTCCTTTAAGGATGACGGCACGTACAAAACGCGTGCGGAAGT |
| | | GAAATTTGAAGGCGATACCCTGGTAAACCGCATTGAGCTGAAAGGCATTGACTTTAAAGA |
| | | AGACGGCAATATCCTGGGCCATAAGCTGGAATACAATTTTAACAGCCACAATGTTTACAT |
| | | CACCGCCGATAAACAAAAAAATGGCATTAAAGCGAATTTTAAAATTCGCCACAACGTGGA |
| | | GGATGGCAGCGTGCAGCTGGCTGATCACTACCAGCAAAACACTCCAATCGGTGATGGTCC |
| | | TGTTCTGCTGCCAGACAATCACTATCTGAGCACGCAAAGCGTTCTGTCTAAAGATCCGAA |
| | | CGAGAAACGCGATCATATGGTTCTGCTGGAGTTCGTAACCGCAGCGGGCATCACGCATGG |
| | | TATGGATGAACTGTACAAATAATTAATTAAAAGGCCGCTCGAATAACTTCGTATAGCATA |
| | | CATTATACGAAGTTATGCTAGCACGATGCTCTTAGAATAACAACATATACACATTTAATA |
| | | AAAATAATATTTATTAAGAAAATTCAGATTTCACGTACCCATCAATATAAATAAAATAAT |
| | | GATTCCTTACACCGTACCCATATTAAGGAGATTCCACCTTACCCATAAACAATATAAATC |
| | | CAGTAATATCATGTCTGATGATGAACACAAATGGTGTATTAAATTCCAGTTTTTCAGGAG |
| | | ATGATCTCGCCGTAGCTACCATAATAGTAGATGCCTCTGCTACAGTTCCTTGTTCGTCGA |
| | | CATCTATCTTTGCATTCTGAAACATTTTATAAATATATAATGGGTCCCTAGTCATATGTT |
| | | TAAACGACGCATTATCTGGATTAAACATACTAGGAGCCATCATTTCGGCTATCGACTTAA |
| | | TATCCCTCTTATTTTCGATAGAAAATTTAGGGAGTTTAAGATTGTACACTTTATTCCCTA |
| | | ATTGAAACGACCAATAGTCTAATTTTGCAGCCGTAATAGAATCTGTGAAATGGGTCATAT |
| | | TATCACCTATTGCCAGGTACATACTAATATTAGCATCCTTATACGGAAGGCGTACCATAT |
| | | CATATTCTTCGTCATCGATTGTGATTGTATTTCCTTGCAATTTAGTAACTACGTTCATCA |
| | | TGGGAACCGTTTTCGTACCGTACTTATTAGTAAAACTAGCATTGCGTGTTTTAGTGATAT |
| | | CAAACGGATATTGCCATATACCTTTAAAATATATAGTATTAATGATTGCCCATAGAGTAT |
| | | TATTGTCGAGCATATTAGAATCTACTACATTAGACATACCGGATCTACGTTCTACTATAG |
| | | AATTAATTTTATTAACCGCATCTCGTCTAAAGTTTAATCTATATAGGCCGAATCTATGAT |
| | | ATTGTTGATAATACGACGGTTTAATGCACACAGTATTATCTACGAAACTTTGATAAGTTA |
| | | GATCAGTGTACGTATATTTAGATGTTTTCAGCTTAGCTAATCCTGATATTAATTCTGTAA |
| | | ATGCTGGACCCAGATCTCTTTTTCTCAAATCCATAG |
| 5 | Gagpol | ATGGGTGCGAGAGCGTCAGTATTAAGCGGGGGAGAATTAGATCGATGGGAAAAAATTCGG |
| | | TTAAGGCCAGGGGGAAAGAAAAAATATAAATTAAAACATATAGTATGGGCAAGCAGGGAG |
| | | CTAGAACGATTCGCAGTTAATCCTGGCCTGTTAGAAACATCAGAAGGCTGTAGACAAATA |
| | | CTGGGACAGCTACAACCATCCCTTCAGACAGGATCAGAAGAACTTAGATCATTATATAAT |
| | | ACAGTAGCAACCCTCTATTGTGTGCATCAAAGGATAGAGATAAAAGACACCAAGGAAGCT |
| | | TTAGACAAGATAGAGGAAGAGCAAAACAAAAGTAAGAAAAAAGCACAGCAAGCAGCAGCT |
| | | GACACAGGACACAGCAATCAGGTCAGCCAAAATTACCCTATAGTGCAGAACATCCAGGGG |
| | | CAAATGGTACATCAGGCCATATCACCTAGAACTTTAAATGCATGGGTAAAAGTAGTAGAA |
| | | GAGAAGGCTTTCAGCCCAGAAGTGATACCCATGTTTTCAGCATTATCAGAAGGAGCCACC |
| | | CCACAAGATTTAAACACCATGCTAAACACAGTGGGGGGACATCAAGCAGCCATGCAAATG |
| | | TTAAAAGAGACCATCAATGAGGAAGCTGCAGAATGGGATAGAGTGCATCCAGTGCATGCA |
| | | GGGCCTATTGCACCAGGCCAGATGAGAGAACCAAGGGGAAGTGACATAGCAGGAACTACT |
| | | AGTACCCTTCAGGAACAAATAGGATGGATGACACATAATCCACCTATCCCAGTAGGAGAA |
| | | ATCTATAAAAGATGGATAATCCTGGGATTAAATAAAATAGTAAGAATGTATAGCCCTACC |
| | | AGCATTCTGGACATAAGACAAGGACCAAAGGAACCCTTTAGAGACTATGTAGACCGATTC |
| | | TATAAAACTCTAAGAGCCGAGCAAGCTTCACAAGAGGTAAAAAATTGGATGACAGAAACC |
| | | TTGTTGGTCCAAAATGCGAACCCAGATTGTAAGACTATTTTAAAAGCATTGGGACCAGGA |
| | | GCGACACTAGAAGAAATGATGACAGCATGTCAGGGAGTGGGGGGACCCGGCCATAAAGCA |
| | | AGAGTTTTGGCTGAAGCAATGAGCCAAGTAACAAATCCAGCTACCATAATGATACAGAAA |
| | | GGCAATTTTAGGAACCAAAGAAAGACTGTTAAGTGTTTCAATTGTGGCAAAGAAGGGCAC |
| | | ATAGCCAAAAATTGCAGGGCCCCTAGGAAAAAGGGCTGTTGGAAATGTGGAAAGGAAGGA |
| | | CACCAAATGAAAGATTGTACTGAGAGACAGGCTAATTTCTTAGGGAAGATCTGGCCTTCC |
| | | CACAAGGGAAGGCCAGGGAATTTTCTTCAGAGCAGACCAGAGCCAACAGCCCCACCAGAA |
| | | GAGAGCTTCAGGTTTGGGGAAGAGACAACAACTCCCTCTCAGAAGCAGGAGCCGATAGAC |
| | | AAGGAACTGTATCCTTTAGCTTCCCTCAGATCACTCTTTGGCAGCGACCCCTCGTCACAA |
| | | TAAAGATAGGGGGGCAATTAAAGGAAGCTCTATTAGATACAGGAGCAGATGATACAGTAT |
| | | TAGAAGAAATGAATTTGCCAGGAAGATGGAAACCAAAAATGATAGGGGGAATTGGAGGTT |
| | | TTATCAAAGTAAGACAGTATGATCAGATACTCATAGAAATCTGCGGACATAAAGCTATAG |
| | | GTACAGTATTAGTAGGACCTACACCTGTCAACATAATTGGAAGAAATCTGTTGACTCAGA |
| | | TTGGCTGCACTTTAAATTTTCCCATTAGTCCTATTGAGACTGTACCAGTAAAATTAAAGC |
| | | CAGGAATGGATGGCCCAAAAGTTAAACAATGGCCATTGACAGAAGAAAAAATAAAAGCAT |
| | | TAGTAGAAATTTGTACAGAAATGGAAAAGGAAGGAAAAATTTCAAAAATTGGGCCTGAAA |
| | | ATCCATACAATACTCCAGTATTTGCCATAAAGAAAAAAGACAGTACTAAATGGAGAAAAT |
| | | TAGTAGATTTCAGAGAACTTAATAAGAGAACTCAAGATTTCTGGGAAGTTCAATTAGGAA |
| | | TACCACATCCTGCAGGGTTAAAACAGAAAAAATCAGTAACAGTACTGGATGTGGGCGATG |
| | | CATATTTCTCAGTTCCCTTAGATAAAGACTTCAGGAAGTATACTGCATTTACCATACCTA |
| | | GTATAAACAATGAGACACCAGGGATTAGATATCAGTACAATGTGCTTCCACAGGGATGGA |
| | | AAGGATCACCAGCAATATTCCAGTGTAGCATGACAAAAATCTTAGAGCCTTTTAGAAAAC |
| | | AAAATCCAGACATAGTCATCTATCAATACATGGATGATTTGTATGTAGGATCTGACTTAG |
| | | AAATAGGGCAGCATAGAACAAAAATAGAGGAACTGAGACAACATCTGTTGAGGTGGGGAT |
| | | TTACCACACCAGACAAAAAACATCAGAAAGAACCTCCATTCCTTTGGATGGGTTATGAAC |
| | | TCCATCCTGATAAATGGACAGTACAGCCTATAGTGCTGCCAGAAAAGGACAGCTGGACTG |
| | | TCAATGACATACAGAAATTAGTGGGAAAATTGAATTGGGCAAGTCAGATTTATGCAGGGA |
| | | TTAAAGTAAGGCAATTATGTAAACTTCTTAGGGGAACCAAAGCACTAACAGAAGTAGTAC |
| | | CACTAACAGAAGAAGCAGAGCTAGAACTGGCAGAAAACAGGGAGATTCTAAAAGAACCGG |
| | | TACATGGAGTGTATTATGACCCATCAAAAGACTTAATAGCAGAAATACAGAAGCAGGGGC |
| | | AAGGCCAATGGACATATCAAATTTATCAAGAGCCATTTAAAAATCTGAAAACAGGAAAGT |
| | | ATGCAAGAATGAAGGGTGCCCACACTAATGATGTGAAACAATTAACAGAGGCAGTACAAA |
| | | AAATAGCCACAGAAAGCATAGTAATATGGGGAAAGACTCCTAAATTTAAATTACCCATAC |
| | | AAAAGGAAACATGGGAAGCATGGTGGACAGAGTATTGGCAAGCCACCTGGATTCCTGAGT |
| | | GGGAGTTTGTCAATACCCCTCCCTTAGTGAAGTTATGGTACCAGTTAGAGAAAGAACCCA |
| | | TAATAGGAGCAGAAACTTTCTATGTAGATGGGGCAGCCAATAGGGAAACTAAATTAGGAA |
| | | AAGCAGGATATGTAACTGACAGAGGAAGACAAAAAGTTGTCCCCCTAACGGACACAACAA |
| | | ATCAGAAGACTGAGTTACAAGCAATTCATCTAGCTTTGCAGGATTCGGGATTAGAAGTAA |
| | | ACATAGTGACAGACTCACAATATGCATTGGGAATCATTCAAGCACAACCAGATAAGAGTG |
| | | AATCAGAGTTAGTCAGTCAAATAATAGAGCAGTTAATAAAAAAGGAAAAAGTCTACCTGG |
| | | CATGGGTACCAGCACACAAAGGAATTGGAGGAAATGAACAAGTAGATAAATTGGTCAGTG |
| | | CTGGAATCAGGAAAGTACTATTCTTAGATGGAATAGATAAGGCCCAAGAAGAACATGAGA |
| | | AATATCACAGTAATTGGAGAGCAATGGCTAGTGATTTTAACCTACCACCTGTAGTAGCAA |
| | | AAGAAATAGTAGCCAGCTGTGATAAATGTCAGCTAAAAGGGGAAGCCATGCATGGACAAG |
| | | TAGACTGTAGCCCAGGAATATGGCAGCTAGATTGTACACATTTAGAAGGAAAAGTTATCT |
| | | TGGTAGCAGTTCATGTAGCCAGTGGATATATAGAAGCAGAAGTAATTCCAGCAGAGACAG |
| | | GGCAAGAAACAGCATACTTCCTCTTAAAATTAGCAGGAAGATGGCCAGTAAAAACAGTAC |
| | | ATACAGACAATGGCAGCAATTTCACCAGTACTACAGTTAAGGCCGCCTGTTGGTGGGCGG |
| | | GGATCAAGCAGGAATTTGGCATTCCCTACAATCCCCAAAGTCAAGGAGTAATAGAATCTA |
| | | TGAATAAAGAATTAAAGAAAATTATAGGACAGGTAAGAGATCAGGCTGAACATCTTAAGA |
| | | CAGCAGTACAAATGGCAGTATTCATCCACAATTTTAAAAGAAAAGGGGGGATTGGGGGGT |
| | | ACAGTGCAGGGGAAAGAATAGTAGACATAATAGCAACAGACATACAAACTAAAGAATTAC |
| | | AAAAACAAATTACAAAAATTCAAAATTTTCGGGTTTATTACAGGGACAGCAGAGATCCAG |
| | | TTTGGAAAGGACCAGCAAAGCTCCTCTGGAAAGGTGAAGGGGCAGTAGTAATACAAGATA |
| | | ATAGTGACATAAAAGTAGTGCCAAGAAGAAAAGCAAAGATCATCAGGGATTATGGAAAAC |
| | | AGATGGCAGGTGATGATTGTGTGGCAAGTAGACAGGATGAGGATTAA |

### Transfer Construct

The transfer element was inserted between vacwr205 and vacwr206, a region with no late gene transcripts near the far 3′ end of the vaccinia virus genome, Table 1. A map and sequence with elements marked in similar colors as the map is shown in FIG. 4 and Table 4. The transfer construct utilizes a strong vaccinia virus early promoter: GTGATGCGGTTTTGGCAGTACATCAATGGGCGTGGATAGCGGTTTGACTCACGGG GATTTCCAAGTCTCCACCCCATTGACGTCAATGGGAGTTTGTTTTGGCACCAAAA TCAACGGGACTTTCCAAAATGTCGTAACAACTCCGCCCCATTGACGCAAATGGGC GGTAGGCGTGTACGGTGGGAGGTCTATATAAGCAGAGCT (SEQ ID NO: 11) and the human PGK promoter: GGGGTTGGGGTTGCGCCTTTTCCAAGGCAGCCCTGGGTTTGCGCAGGGACGCGGC TGCTCTGGGCGTGGTTCCGGGAAACGCAGCGGCGCCGACCCTGGGTCTCGCACA TTCTTCACGTCCGTTCGCAGCGTCACCCGGATCTTCGCCGCTACCCTTGTGGGCCC CCCGGCGACGCTTCCTGCTCCGCCCCTAAGTCGGGAAGGTTCCTTGCGGTTCGCG GCGTGCCGGACGTGACAAACGGAAGCCGCACGTCTCACTAGTACCCTCGCAGAC GGACAGCGCCAGGGAGCAATGGCAGCGCGCCGACCGCGATGGGCTGTGGCCAAT AGCGGCTGCTCAGCAGGGCGCGCCGAGAGCAGCGGCCGGGAAGGGGCGGTGCG GGAGGCGGGGTGTGGGGCGGTAGTGTGGGCCCTGTTCCTGCCCGCGCGGTGTTCC GCATTCTGCAAGCCTCCGGAGCGCACGTCGGCAGTCGGCTCCCTCGTTGACCGAA TCACCGACCTCTCTCCCCAG (SEQ ID NO: 12)

TABLE 4
Map of the lentiviral transfer construct: vacwr205 (1-905); 5′LTR
(1012-1192); ψ (1239-1364); RRE (1857-2090); CMV promoter (2926-3129);
GFP (3174-3890); cPPT/CTS (4584-4701); hPGK promoter (4750-5260);
PAC (5282-5881); 3'LTR (6009-6242); U5NU (6243-6251); SV40poly(a)
(6323-6331); LoxP (6469-6502 and 7332-7369); rfp (6579-7313);
vacwr206 (7455-8158)

| SEQ | | |
|---|---|---|
| ID | ID | Sequence |
| 6 | LV transfer | CACCCGATGACAATAATGACATGGACGTAGATATTCCGTATTGTGCGACACTAGCTACCG |
| | | CAAATAAAATATACGGTAGCGATAGTATCGAGTTCCACGCCTCCTTCCTACAAAAAATAA |
| | | AAGACGATTTTCAAACTGTAAACTTTAATAATGCTAACCAAACAAAGGAACTAATCAACG |
| | | AATGGGTTAAGACAATGACAAATGGTAAAATTAATTCCTTATTGACTAGTCCGCTATCCA |
| | | TTAATACTCGTATGACAGTTGTTAGCGCCGTCCATTTTAAAGCAATGTGGAAATATCCAT |
| | | TTTCTAAACATCTTACATATACAGACAAGTTTTATATTTCTAAGAATATAGTTACCAGTG |
| | | TTGATATGATGGTGAGCACTGAGAATAACTTGCAATATGTACATATTAATGAATTATTCG |
| | | GAGGATTCTCTATTATCGATATTCCATACGAGGGAAACTCTAGTATGGTAATTATACTAC |
| | | CGGACGACATAGAAGGTATATATAACATAGAAAAAAATATAACAGATGAAAAATTTAAAA |
| | | AATGGTGTGGTATGTTATCTACTAAAAGTATAGACTTGTATATGCCAAAGTTTAAAGTGG |
| | | AAATGACAGAACCGTATAATCTGGTACCGATTTTAGAAAATTTAGGACTTACTAATATAT |
| | | TCGGATATTATGCAGATTTTAGCAAGATGTGTAATGAAACTATCACTGTAGAAAAATTTC |
| | | TACATACGACGTTTATAGATGTTAATGAGGAGTATACAGAAGCATCGGCCGTTACAGGAG |
| | | TATTTATGACTAACTTTTCGATGGTATATCGTACGAAGGTCTACATAAACCATCCATTCA |
| | | TGTACATGATTAAAGACAACACAGGACGTATACTTTTTATAGGGAAATACTGCTATCCGC |
| | | AATAAATATAAACAAATAGACTTTTTATCACGTTTATCTATGTCTAAATATTACAAATAG |
| | | TAATAGTATAAACTAAAGCCTAGGAAAAATTGAAACATTATATTAATCAGCGGGTCTCTC |
| | | TGGTTAGACCAGATCTGAGCCTGGGAGCTCTCTGGCTAACTAGGGAACCCACTGCTTAAG |
| | | CCTCAATAAAGCTTGCCTTGAGTGCTTCAAGTAGTGTGTGCCCGTCTGTTGTGTGACTCT |
| | | GGTAACTAGAGATCCCTCAGACCCTTTTAGTCAGTGTGGAAAATCTCTAGCAGTGGCGCC |
| | | CGAACAGGGACTTGAAAGCGAAAGGGAAACCAGAGGAGCTCTCTCGACGCAGGACTCGGC |
| | | TTGCTGAAGCGCGCACGGCAAGAGGCGAGGGGCGGCGACTGGTGAGTACGCCAAAAATTT |
| | | TGACTAGCGGAGGCTAGAAGGAGAGAGATGGGTGCGAGAGCGTCAGTATTAAGCGGGGGA |
| | | GAATTAGATCGCGATGGGAAAAAATTCGGTTAAGGCCAGGGGGAAAGAAAAAATATAAAT |
| | | TAAAACATATAGTATGGGCAAGCAGGGAGCTAGAACGATTCGCAGTTAATCCTGGCCTGT |
| | | TAGAAACATCAGAAGGCTGTAGACAAATACTGGGACAGCTACAACCATCCCTTCAGACAG |
| | | GATCAGAAGAACTTAGATCATTATATAATACAGTAGCAACCCTCTATTGTGTGCATCAAA |
| | | GGATAGAGATAAAAGACACCAAGGAAGCTTTAGACAAGATAGAGGAAGAGCAAAACAAAA |
| | | GTAAGACCACCGCACAGCAAGCGGCCGCTGATCTTCAGACCTGGAGGAGGAGATATGAGG |
| | | GACAATTGGAGAAGTGAATTATATAAATATAAAGTAGTAAAAATTGAACCATTAGGAGTA |
| | | GCACCCACCAAGGCAAAGAGAAGAGTGGTGCAGAGAGAAAAAAGAGCAGTGGGAATAGGA |
| | | GCTTTGTTCCTTGGGTTCTTGGGAGCAGCAGGAAGCACTATGGGCGCAGCGTCAATGACG |
| | | CTGACGGTACAGGCCAGACAATTATTGTCTGGTATAGTGCAGCAGCAGAACAATTTGCTG |
| | | AGGGCTATTGAGGCGCAACAGCATCTGTTGCAACTCACAGTCTGGGGCATCAAGCAGCTC |
| | | CAGGCAAGAATCCTGGCTGTGGAAAGATACCTAAAGGATCAACAGCTCCTGGGGATTTGG |
| | | GGTTGCTCTGGAAAACTCATTTGCACCACTGCTGTGCCTTGGAATGCTAGTTGGAGTAAT |
| | | AAATCTCTGGAACAGATTTGGAATCACACGACCTGGATGGAGTGGGACAGAGAAATTAAC |
| | | AATTACACAAGCTTAATACACTCCTTAATTGAAGAATCGCAAAACCAGCAAGAAAAGAAT |
| | | GAACAAGAATTATTGGAATTAGATAAATGGGCAAGTTTGTGGAATTGGTTTAACATAACA |
| | | AATTGGCTGTGGTATATAAAATTATTCATAATGATAGTAGGAGGCTTGGTAGGTTTAAGA |
| | | ATAGTTCTTGCTGTACTTTCTATAGTGAATAGAGTTAGGCAGGGATATTCACCATTATCG |
| | | TTTCAGACCCACCTCCCAACCCCGAGGGGACCCGACAGGCCCGAAGGAATAGAAGAAGAA |
| | | GGTGGAGAGAGAGACAGAGACAGATCCATTCGATTAGTGAACGGATCTCGACGGTATCGA |
| | | TCACGAGACTAGCCTCGAGCGGCCGCCCCCTTCACCGAGGGCCTATTTCCCATGATTCCT |
| | | TCATATTTGCATATACGATACAAGGCTGTTAGAGAGATAATTGGAATTAATTTGACTGTA |
| | | AACACAAAGATATTAGTACAAAATACGTGACGTAGAAAGTAATAATTTCTTGGGTAGTTT |
| | | GCAGTTTTAAAATTATGTTTTAAAATGGACTATCATATGCCAAGTACGCCCCCTATTGAC |
| | | GTCAATGACGGTAAATGGCCCGCCTGGCATTATGCCCAGTACATGACCTTATGGGACTTT |
| | | CCTACTTGGCAGTACATCTACGTATTAGTCATCGCTATTACCATGGTGATGCGGTTTTGG |
| | | CAGTACATCAATGGGCGTGGATAGCGGTTTGACTCACGGGGATTTCCAAGTCTCCACCCC |
| | | ATTGACGTCAATGGGAGTTTGTTTTGGCACCAAAATCAACGGGACTTTCCAAAATGTCGT |
| | | AACAACTCCGCCCCATTGACGCAAATGGGCGGTAGGCGTGTACGGTGGGAGGTCTATATA |
| | | AGCAGAGCTGGTTTAGTGAACCGTCAGATCCGCTAGCGCTACCGGTCGCCACCATGCGTA |
| | | AAGGCGAAGAGCTGTTCACTGGTGTCGTCCCTATTCTGGTGGAACTGGATGGTGATGTCA |
| | | ACGGTCATAAGTTTTCCGTGCGTGGCGAGGGTGAAGGTGACGCAACTAATGGTAAACTGA |
| | | CGCTGAAGTTCATCTGTACTACTGGTAAACTGCCGGTCCCTTGGCCGACTCTGGTAACGA |
| | | CGCTGACTTATGGTGTTCAGTGCTTTGCTCGTTATCCGGACCATATGAAGCAGCATGACT |
| | | TCTTCAAGTCCGCCATGCCGGAAGGCTATGTGCAGGAACGCACGATTTCCTTTAAGGATG |
| | | ACGGCACGTACAAAACGCGTGCGGAAGTGAAATTTGAAGGCGATACCCTGGTAAACCGCA |
| | | TTGAGCTGAAAGGCATTGACTTTAAAGAAGACGGCAATATCCTGGGCCATAAGCTGGAAT |
| | | ACAATTTTAACAGCCACAATGTTTACATCACCGCCGATAAACAAAAAAATGGCATTAAAG |
| | | CGAATTTTAAAATTCGCCACAACGTGGAGGATGGCAGCGTGCAGCTGGCTGATCACTACC |
| | | AGCAAAACACTCCAATCGGTGATGGTCCTGTTCTGCTGCCAGACAATCACTATCTGAGCA |
| | | CGCAAAGCGTTCTGTCTAAAGATCCGAACGAGAAACGCGATCATATGGTTCTGCTGGAGT |
| | | TCGTAACCGCAGCGGGCATCACGCATGGTATGGATGAACTGTACAAATAAGAATTCGATA |
| | | TCAAGCTTATCGATAATCAACCTCTGGATTACAAAATTTGTGAAAGATTGACTGGTATTC |
| | | TTAACTATGTTGCTCCTTTTACGCTATGTGGATACGCTGCTTTAATGCCTTTGTATCATG |
| | | CTATTGCTTCCCGTATGGCTTTCATTTTCTCCTCCTTGTATAAATCCTGGTTGCTGTCTC |
| | | TTTATGAGGAGTTGTGGCCCGTTGTCAGGCAACGTGGCGTGGTGTGCACTGTGTTTGCTG |
| | | ACGCAACCCCCACTGGTTGGGGCATTGCCACCACCTGTCAGCTCCTTTCCGGGACTTTCG |
| | | CTTTCCCCCTCCCTATTGCCACGGCGGAACTCATCGCCGCCTGCCTTGCCCGCTGCTGGA |
| | | CAGGGGCTCGGCTGTTGGGCACTGACAATTCCGTGGTGTTGTCGGGGAAATCATCGTCCT |
| | | TTCCTTGGCTGCTCGCCTGTGTTGCCACCTGGATTCTGCGCGGGACGTCCTTCTGCTACG |
| | | TCCCTTCGGCCCTCAATCCAGCGGACCTTCCTTCCCGCGGCCTGCTGCCGGCTCTGCGGC |
| | | CTCTTCCGCGTCTTCGCCTTCGCCCTCAGACGAGTCGGATCTCCCTTTGGGCCGCCTCCC |
| | | CGCATCGATACCGTCCGGACTCAGATCTCGAGCTCAAGCTTCGAATTCTCGACCTCGAGA |
| | | CAAATGGCAGTATTCATCCACAATTTTAAAAGAAAAGGGGGGATTGGGGGGTACAGTGCA |
| | | GGGGAAAGAATAGTAGACATAATAGCAACAGACATACAAACTAAAGAATTACAAAAACAA |
| | | ATTACAAAAATTCAAAATTTTCGGGTTTATTACAGGGACAGCAGAGATCCACTTTGGCCG |
| | | CGGCTCGAGGGGGTTGGGGTTGCGCCTTTTCCAAGGCAGCCCTGGGTTTGCGCAGGGACG |
| | | CGGCTGCTCTGGGCGTGGTTCCGGGAAACGCAGCGGCGCCGACCCTGGGTCTCGCACATT |
| | | CTTCACGTCCGTTCGCAGCGTCACCCGGATCTTCGCCGCTACCCTTGTGGGCCCCCCGGC |
| | | GACGCTTCCTGCTCCGCCCCTAAGTCGGGAAGGTTCCTTGCGGTTCGCGGCGTGCCGGAC |
| | | GTGACAAACGGAAGCCGCACGTCTCACTAGTACCCTCGCAGACGGACAGCGCCAGGGAGC |
| | | AATGGCAGCGCGCCGACCGCGATGGGCTGTGGCCAATAGCGGCTGCTCAGCAGGGCGCGC |
| | | CGAGAGCAGCGGCCGGGAAGGGGCGGTGCGGGAGGCGGGGTGTGGGGCGGTAGTGTGGGC |
| | | CCTGTTCCTGCCCGCGCGGTGTTCCGCATTCTGCAAGCCTCCGGAGCGCACGTCGGCAGT |
| | | CGGCTCCCTCGTTGACCGAATCACCGACCTCTCTCCCCAGGGGGATCCACCGGAGCTTAC |
| | | CATGACCGAGTACAAGCCCACGGTGCGCCTCGCCACCCGCGACGACGTCCCCAGGGCCGT |
| | | ACGCACCCTCGCCGCCGCGTTCGCCGACTACCCCGCCACGCGCCACACCGTCGATCCGGA |
| | | CCGCCACATCGAGCGGGTCACCGAGCTGCAAGAACTCTTCCTCACGCGCGTCGGGCTCGA |
| | | CATCGGCAAGGTGTGGGTCGCGGACGACGGCGCCGCGGTGGCGGTCTGGACCACGCCGGA |
| | | GAGCGTCGAAGCGGGGGCGGTGTTCGCCGAGATCGGCCCGCGCATGGCCGAGTTGAGCGG |
| | | TTCCCGGCTGGCCGCGCAGCAACAGATGGAAGGCCTCCTGGCGCCGCACCGGCCCAAGGA |
| | | GCCCGCGTGGTTCCTGGCCACCGTCGGCGTCTCGCCCGACCACCAGGGCAAGGGTCTGGG |
| | | CAGCGCCGTCGTGCTCCCCGGAGTGGAGGCGGCCGAGCGCGCCGGGGTGCCCGCCTTCCT |
| | | GGAGACCTCCGCGCCCCGCAACCTCCCCTTCTACGAGCGGCTCGGCTTCACCGTCACCGC |
| | | CGACGTCGAGGTGCCCGAAGGACCGCGCACCTGGTGCATGACCCGCAAGCCCGGTGCCTG |
| | | ACGCCCGCCCCACGACCCGCAGCGCCCGACCGAAAGGAGCGCACGACCCCATGCATCGGT |
| | | ACCTTTAAGACCAATGACTTACAAGGCAGCTGTAGATCTTAGCCACTTCTTAAAAGAAAA |
| | | GGGGGGACTGGAAGGGCTAATTCACTCCCAACGAAGACAAGATCTGCTTTTTGCTTGTAC |
| | | TGGGTCTCTCTGGTTAGACCAGATCTGAGCCTGGGAGCTCTCTGGCTAACTAGGGAACCC |
| | | ACTGCTTAAGCCTCAATAAAGCTTGCCTTGAGTGCTTCAAGTAGTGTGTGCCCGTCTGTT |
| | | GTGTGACTCTGGTAACTAGAGATCCCTCAGACCCTTTTAGTCAGTGTGGAAAATCTCTAG |
| | | CAAATTTTTATGTAGTAGTTCATGTCATCTTATTATTCAGTATTTATAACTTGCAAAGAA |
| | | ATGAATATCAGAGAGTGAGAGGAATTTTTATAACTTGTTTATTGCAGCTTATAATGGTTA |
| | | CAAATAAAGCAATAGCATCACAAATTTCACAAATAAAGCATTTTTTTCACTGCATTCTAG |
| | | TTGTGGTTTGTCCAAACTCATCAATGTATCTTAGGTATAGCCTCTAGAATAACTTCGTAT |
| | | AGCATACATTATACGAAGTTATAATTCTGCAGATGGATCATAAAAATATAGTAGAATTTC |
| | | ATTTTGTTTTTTTCTATGCTATAAATAGCTTCTCGAGCATGGTGTCTAAGGGCGAAGAGC |
| | | TGATTAAGGAGAACATGCACATGAAGCTGTACATGGAGGGCACCGTGAACAACCACCACT |
| | | TCAAGTGCACATCCGAGGGCGAAGGCAAGCCCTACGAGGGCACCCAGACCATGAGAATCA |
| | | AGGTGGTCGAGGGCGGCCCTCTCCCCTTCGCCTTCGACATCCTGGCTACCAGCTTCATGT |
| | | ACGGCAGCAGAACCTTCATCAACCACACCCAGGGCATCCCCGATTTCTTTAAGCAGTCCT |
| | | TCCCTGAGGGCTTCACATGGGAGAGAGTCACCACATACGAAGACGGGGGCGTGCTGACCG |
| | | CTACCCAGGACACCAGCCTCCAGGACGGCTGCCTCATCTACAACGTCAAGATCAGAGGGG |
| | | TGAACTTCCCATCCAACGGCCCTGTGATGCAGAAGAAAACACTCGGCTGGGAGGCCAACA |
| | | CCGAGATGCTGTACCCCGCTGACGGCGGCCTGGAAGGCAGAACCGACATGGCCCTGAAGC |
| | | TCGTGGGCGGGGGCCACCTGATCTGCAACTTCAAGACCACATACAGATCCAAGAAACCCG |
| | | CTAAGAACCTCAAGATGCCCGGCGTCTACTATGTGGACCACAGACTGGAAAGAATCAAGG |
| | | AGGCCGACAAAGAGACCTACGTCGAGCAGCACGAGGTGGCTGTGGCCAGATACTGCGACC |
| | | TCCCTAGCAAACTGGGGCACAAACTTAATGGCATGGACGAGCTGTACAAGTAATTAATTA |
| | | AGGCCGCTCGAATAACTTCGTATAGCATACATTATACGAAGTTATCTGATAATACTTAAA |
| | | AAAATAATAATATCATTTACAATTAATAGTATAAACTAAAAATTAAACAAATCGTTATTA |
| | | TAAGTAATATCAAAATGATGATATACGGATTAATAGCGTGTCTTATATTCGTGACTTCAT |
| | | CCATCGCTAGTCCACTTTATATTCCCGTTATTCCACCCATTTCGGAAGATAAATCGTTCA |
| | | ATAGTGTAGAGGTATTAGTTTCCTTGTTTAGAGATGACCAAAAAGACTATACGGTAACTT |
| | | CTCAGTTCAATAACTACACTATCGATACCAAAGACTGGACTATCGGCGTACTATCCACAC |
| | | CTGATGGTTTGGATATACCATTGACTAATATAACTTATTGGTCACGGTTTACTATAGGTC |
| | | GTGCATTGTTCAAATCAGAGTCTGAGGATATTTTCCAAAAGAAAATGAGTATTCTAGGTG |
| | | TTTCTATAGAATGTAAGAAGTCGTCGACATTACTTACTTTTTTGACCGTGCGTAAAATGA |
| | | CTCGAGTATTTAATAAATTTCCAGATATGGCTTATTATCGAGGAGACTGTTTAAAAGCCG |
| | | TTTATGTAACAATGACTTATAAAAATACTAAAACTGGAGAGACTGATTACACGTACCTCT |
| | | CTAATGGGGGGTTGCCTGCATACTATCGTAATGGGGTCGATGGTTGATTATTGATTAGTA |
| | | TATTCCTTATTCTTTTTATTCACACAAAAAGAACATTTTTATAAACATGAAACCACTGTC |
| | | TAAATGTAATTATGATCTTGATTTATAGATGAAGATCAGCCTTTAGAGGATTTTAACC |

### Example 2: Generation of a Viral Construct Comprising a Non-Replicating Lentivirus

Viral constructs were created by combining short DNA fragment “gBlocks” synthesized by Integrated DNA Technologies, primer extensions, and/or by full plasmid synthesis products from GenScript. BS-C-1 cells were transfected with the completed plasmids and then infected with wildtype vaccinia virus of the Western Reserve strain obtained from ATCC. Constructs were added and selected for sequentially starting with the env construct, followed by packaging construct, and finally the transfer construct. In between each addition, the vaccinia virus reporters were removed by growing on Cre+ cells to remove sfGFP or TagRFP-T, and then reporter-negative virus plaques were selected.

### Expression of Gag/Pol

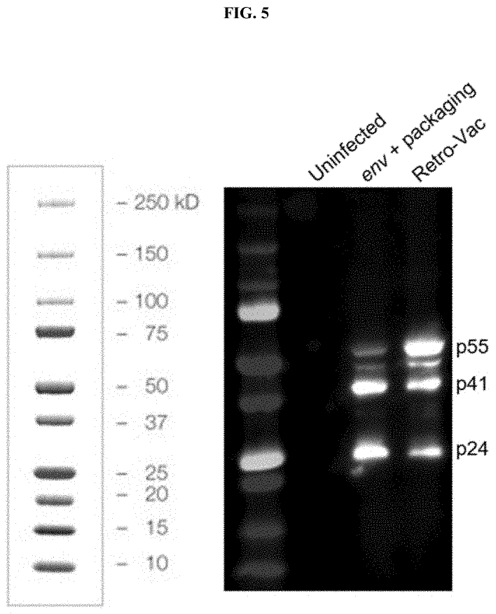

Detection of GAG expression was performed by Western blot. BS-C-1 cells were infected with the indicated viruses at 5 multiplicity of infection (MOI). After 24 h, cells were processed for Western blot and Gag was visualized using the HRP-Conjugated Anti-HIV-1 Gag antibody from ViroLabs (Cat #HIVGAG-HRP, Lot #0516P4). The expression of gagpol from Retro-Vac resulted in a fully mature and processed Gag protein, including p55, p41, and p24 as shown in FIG. 5. This indicated that the HIV protease (N-terminal of pol) was expressed and active because a successful −1 frameshift induced by the HIV-1 ribosomal frameshift signal near the beginning of the pol sequence. It is possible that host factors that assist this frameshift such as eukaryotic release factor 1 (eRF1) may be present in the cytoplasm and vaccinia virus factory. The frameshift is thought to occur in HIV-1 at 5% of total transcription events, and it is important that this ratio stay near 5% to create competent virus.

### Expression of VSV-G

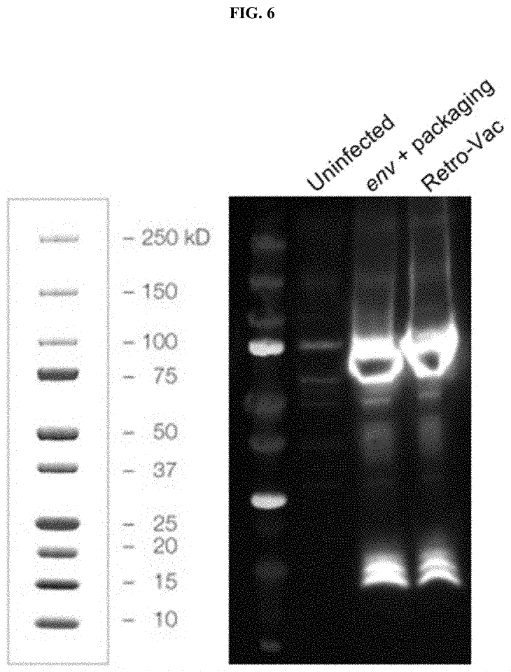

Successful expression of VSV-G from Retro-Vac was detected on Western blot. BS-C-1 cells were infected with the indicated viruses at 5 MOI. After 24 h, cells were processed for Western blot and VSV-G was visualized using anti-VSV-G antibody (clone poly29039) from Bio legend (Cat #903901, Lot #B232548) as shown in FIG. 6. VSV-G is predicted to be 57.4 kDa.

### Reporter Genes Indicate Successful Retrovirus Production

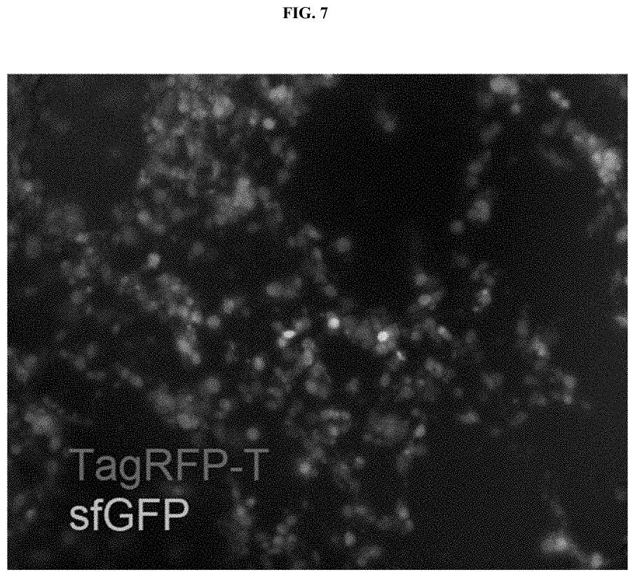

Once all lentiviral constructs were included in the same vaccinia virus lineage, the final product was grown on the common lentiviral growth cell line HEK293. After 24 h of infection, TagRFP-T reporter from the vaccinia virus was visualized within viral plaques using fluorescence microscopy as shown in FIG. 7. In addition, sfGFP reporter within the retroviral transfer construct was detected on cells scattered within viral plaques. This indicated that retrovirus was being created from vaccinia virus-infected cells and was transducing neighboring cells.

### Example 3: Collection of Viral Particles

To isolate viral particles, about 12×10⁶ 293 T cells are plated in 20 ml on a 15 cm2 plate 24 hours before transfection. In general, two 15 cm plates are used. Cells should be well-maintained and of relatively low passage number. The following steps are done 1 plate at a time. 2 ml of 2×HBS is added dropwise to the DNA mixture while bubbling with a Pasteur pipette. Upon completion, allow the mixture to bubble for 12-15 seconds. Take plate of 293T out of the incubator (plate remains in incubator for as long as possible) and add transfection mixture dropwise all over the plate. Gently swirl plate from front to back and return immediately to incubator. 3.5 to 4 hours later, remove media, wash 2× with 10 ml warm PBS, and add 20 ml warm D10 onto plate and place in incubator. 36-48 hours after transfection, harvest viral supernatant and spin at 2000 rpm, 7 min at 4° C. in a 50 ml tube. Filter viral SN through 0.45 um filter. Add 35 ml of filtered supernatant to an ultracentrifuge tube. Balance tubes with additional media. Cover tubes with small piece of parafilm. (It is useful to titer some of the leftover supernatant to determine if there is loss of virus during concentration.) Spin tubes using a SW-28 rotor at 25,000 rpm, 90 min, 4° C. Decant liquid and leave tube upside down on Kimwipe for 10 min. Aspirate remaining media being careful not to touch bottom of tube. Add 15 μl cold PBS (for embryo infections, or any volume you wish) and leave tube at 4° C. O/N with no shaking. To resuspend, hold tube at angle and pipet fluid over pellet 20 times, being careful not to touch pellet with tip. It is expected that the pellet not be resuspended after this is complete. This pellet does not contain virus and can be discarded. Aliquot or use virus. Virus should be aliquoted, flash-frozen in liquid nitrogen and stored at −80. There should be no change in titer with freezing concentrated virus. Avoid multiple freeze-thaws.

In another experiment cells are infected with virus generated from vectors described herein. To preferentially select production-enhanced mutants, medium from flasks infected with the virus pool is added to non-infected flasks. Clonal plaques are isolated after several rounds of serial infection.

### Example 4: Cell Viability Assays

In one experiment HeLa cells are grown to confluency in 6-well plates and infected with 1 MOI of Retro-Vac. After 24 h, 1 mL of supernatant is removed and centrifuged at 800 g, 500 ul of supernatant is then removed and used for viral plaque assay. During the serial dilution, samples are treated with anti-L1 NR-45114 antibody or VIG then incubated for 1 h at 37° C. Dilutions are then added to confluent 6 well plates of BS-C-40 cells for plaque assay. After 1.5 h, media was replaced with CM10 with 3% CMC. After 48 h, cells are stained with crystal violet to count viral plaques to determine the titer of virus in the HeLa cell supernatant and the blocking ability of neutralizing antibodies.

In another experiment, HCT116 or MC38 cell lines are seeded in 96-well plates and allowed to grow until 90% confluent. Cells are then infected with 1 MOI of virus generated from viral vectors provided herein, for example comprising sequences provided herein. Each day, at 24 h intervals, cell viability is tested with the CellTiter 96 Aqueous Non-radioactive Cell Proliferation Kit from Promega. Relative viability is calculated by removing the blank value average from all wells, calculating the average value of the uninfected control group, then calculating the relative value of each infected well as (A490/average of uninfected wells).

### Example 5: Testing of Oncolytic Potential of a Viral Vector in Mice

The aim of this study is to explore the effects of provided viral vectors in a murine mouse model of cancer, in comparison with other vectors, such as control vectors.

In one experiment, a viral vector that produces a non-replicating lentivirus is compared with a vehicle (buffered saline) control, and another modified vaccinia virus that does not generate lentiviral vector. A single dose of either one of the two viruses (1×10⁷ PFU) or the saline control is administered to treat BALB/c mice implanted subcutaneously with pre-established RENCA tumors. Tumor volumes are monitored by caliper measurement.

In another experiment, a different mouse tumor model, C57/BL6 mice bearing subcutaneous B16 tumors, are used to test the effects of the Retro-Vac virus. A single intravenous injection of either virus is given to the mice (1×10⁸ PFU/mouse, n=5 per group/time point) 96 hours after the tumor implantation, and then the mice are sacrificed 1 day or 2 days after the injection. Tumors are collected for quantification of the number of viral genomes per gram of tumor by qPCR.

In one experiment, the Retro-Vac vector is tested in mice bearing orthotopic (mammary fat pad) 4T1 tumors subcutaneously. Each mouse is treated with a single injection of the Retro-Vac vector at a dose of 1×10⁸ PFU. The number of viruses in the tumors is quantified by bioluminescence imaging and measurement of the luciferase activity in vivo, every day after the injection for three days. The therapeutic effects of the viruses are also examined. Tumor volume is monitored along with mouse survival percentage.

In another experiment, the same viruses are tested in a different tumor model, C57/BL6 mice bearing B16 tumors subcutaneously.

In another experiment, viral replication capability for virus comprising a non-replicating lentivirus in different cancer cell lines in vitro is examined with plaque assays. Plaque-forming are quantified every 24 hours after addition of the viruses to the cell line. Results will be monitored to determine whether an enhanced effect in vivo can be detected.

In an effort to start to understand the mechanisms underlying the enhanced therapeutic effects conferred by CXCR4 expression, experiments are conducted using transgenic mice with B-cells depleted (referred to herein as JH). Viruses as described above are administered to Balb/C mice having B-Cells and JH mice without B-cells, both of which are implanted subcutaneously with pre-established 4T1 tumors. Bioluminescence imaging radiance is used to determine whether the Retro-Vac virus displays increased accumulation in the tumors. Flow cytometry experiments are also performed to determine whether there is entry of B cells into the tumors, but not other organs, like spleen or lymph nodes (LN), in BALB/c mice bearing 4T1 tumor subcutaneously.

In another experiment T cells are collected from the mice that have been implanted with 4T1 tumors subcutaneously and subsequently treated as described above, and their immune activity is examined by ELISpot assays that test their interferon-γ (IFNgamma) release in response to different immunogens. The tested T cells are recovered from spleens 14 days after the virus injection.

In one experiment, BALB/c mice bearing RENCA tumors subcutaneously are treated with a single intravenous injection (1×10⁷ PFU) of Retro-Vac. Tumors are harvested 24 hours later, and the number of viral genomes per milligram of tissue quantified by qPCR.

In another experiment, BALB/c mice bearing RENCA tumors subcutaneously are treated with intravenous injections (1×10⁷ PFU) of one of the same four modified viruses on day 1 and 4, and tumor volume is monitored as described above.

In another experiment lentivirus expression is assayed in tumor samples for 14 days after the virus injection as described above. It is expected that long term expression of the PD 1 checkpoint inhibitor is modulated using this system.

### Example 6: Retro-Vac with Gammaretrovirus Components

An exemplary Retro-Vac of this disclosure is designed, where the lentiviral components of the packaging and transfer constructs are replaced with those of a gamma retrovirus (e.g., a moloney murine leukemia virus (MMLV)). Relative to lentiviral systems, gammaretroviruses like MMLV have selective infection and integration only with actively dividing cells. Thus, this approach can be expected to increase the safety profile and specificity of retroviral targeting by restricting infection to only actively dividing neoplastic cells.

### Example 7: Retro-Vac with a T7 Polymerase Expression System

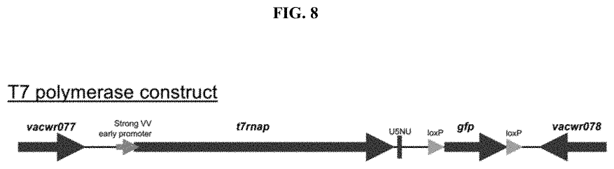

In poxvirus systems, early gene transcription can produce low copy, shorter-lived, and less stable viral mRNA, while intermediate and late gene transcription can produce the most abundant, high copy, and stable viral mRNA. mRNA from early genes have clean transcription start sites (TSS) and end at a specific viral termination site. Intermediate and late genes, however, are modified to include a 5 poly(A) cap and can have unregulated and highly variable termination. Thus, to craft RNA suitable as retroviral gRNA from a lentiviral transfer construct, only the early gene transcription can usually be relied upon considering a precise TSS and termination are essential to retroviral RNA packaging. This limits transcription of the transfer construct to a low copy, low stability system. To circumvent these restrictions, a vaccinia virus is generated with an expression system based on the DNA-dependent RNA polymerase from T7 bacteriophage. This version of the retro-vac system can also express the T7 polymerase (SEQ ID No. 8 provides sequence of T7 RNA polymerase gene) which binds with high specificity to an 18 bp promoter upstream of the lentiviral transfer construct. Transcription can continue until stopping at a highly efficient termination site. This system generates much higher RNA copies relative to early-only VV transcription, and can also allow for greater control of RNA ends. Since transcription of the transfer construct can be a rate-limiting step in lentiviral production, the addition of this T7 polymerase system to retro-vac is contemplated to significantly increase lentiviral titers. An example of such a construct is provided in FIG. 8. Example sequences for T7 RNA polymerase containing construct is provided as SEQ ID No. 7.

| SEQ | | |
|---|---|---|
| ID | ID | Sequence |
| 7 | T7RNA | CATTTATACATATTCCTGGAGATACACTGTTTAAAATTAGCGAGGTATTTATTCATAATA |
| | polymerase | AGATAAATCCATCTTCCAGAATGGCATACATAGAAGAAGAAAAGAGAAATTTAGTTACTG |
| | plasmid | CTATACAGATGTATACTCCTCCTGATGGATCATCCGGTATAGTCTTTGTGGCATCCGTTG |
| | | CACAGTGTCACGAATATAAATCATATTTAGAAAAAAGATTACCGTATGATATGTATATTA |
| | | TTCATGGTAAGGTCTTAGATATAGACGAAATATTAGAAAAAGTGTATTCATCACCTAATG |
| | | TATCGATAATTATTTCTACTCCTTATTTGGAATCCAGCGTTACTATACGCAATGTTACAC |
| | | ACATTTATGATATGGGTAAAGTTTTTGTCCCCGCTCCTTTTGGAGGATCGCAAGAATTTA |
| | | TTTCTAAATCTATGAGAGATCAACGAAAAGGAAGAGTAGGAAGAGTTAATCCTGGTACAT |
| | | ACGTCTATTTCTATGATCTGTCTTATATGAAGTCTATACAGCGAATAGATTCAGAATTTC |
| | | TACATAATTATATATTGTACGCTAATAAGTTTAATCTAACACTCCCCGAAGATTTGTTTA |
| | | TAATCCCTACAAATTTGGATATTCTATGGCGTACAAAGGAATATATAGACTCGTTCGATA |
| | | TTAGTACAGAAACATGGAATAAATTATTATCCAATTATTATATGAAGATGATAGAGTATG |
| | | CTAAACTTTATGTACTAAGTCCTATTCTCGCTGAGGAGTTGGATAACTTTGAGAGGACGG |
| | | GAGAATTAACTAGTATTGTACGAGAAGCCATTTTATCTCTAAATTTACGAATTAAGATTT |
| | | TAAATTTTAAACATAAAGATGATGATACGTATATACACTTTTGTAAAATATTATTCGGTG |
| | | TCTATAACGGAACAAACGCTACTATATATTATCATAGACCTCTAACGGGATATATGAATA |
| | | TGATTTCAGATACTATATTTGTTCCTGTAGATAATAACTAAAAGGCCGGCCGCTAGCTCA |
| | | CTAATTCCAAACCCACCCGCTTTTTATAGTAAGTTTTTCACCCATAAATAATAAATACAA |
| | | TAATTAATTTCTCGTAAAAGTAGAAAATATATTCTAATTTATTGCACGGTAAGGAAGTAG |
| | | ATCATAAGGATCCATGAACACAATTAACATAGCCAAGAACGACTTCAGTGACATTGAGTT |
| | | GGCTGCCATCCCGTTTAATACCTTAGCAGATCACTACGGAGAGCGACTTGCGAGAGAGCA |
| | | ATTGGCCCTTGAGCATGAATCCTACGAAATGGGAGAAGCGCGATTCCGTAAGATGTTCGA |
| | | GAGACAATTAAAAGCCGGTGAAGTAGCGGACAACGCCGCAGCAAAGCCACTTATCACAAC |
| | | ACTACTTCCGAAGATGATAGCACGTATTAACGATTGGTTTGAGGAAGTAAAGGCCAAGAG |
| | | AGGTAAGAGACCGACGGCTTTCCAATTCCTACAAGAGATCAAGCCTGAAGCGGTGGCGTA |
| | | TATAACTATCAAAACGACCCTAGCGTGCCTAACTTCTGCCGATAACACGACGGTACAGGC |
| | | TGTCGCTTCAGCGATTGGAAGAGCGATAGAAGATGAAGCACGTTTCGGACGAATCCGTGA |
| | | TCTAGAAGCTAAACATTTTAAAAAGAACGTTGAGGAGCAGTTGAACAAACGAGTAGGTCA |
| | | CGTGTACAAAAAGGCATTCATGCAAGTGGTAGAAGCCGACATGCTATCCAAGGGATTGCT |
| | | TGGAGGTGAGGCCTGGTCGAGTTGGCACAAAGAGGACTCTATCCACGTCGGTGTTAGATG |
| | | CATAGAGATGCTAATAGAGTCGACAGGTATGGTTTCCCTACATCGTCAAAATGCTGGAGT |
| | | TGTAGGTCAGGATAGTGAAACGATCGAGCTAGCGCCGGAATACGCAGAGGCGATTGCGAC |
| | | GCGAGCTGGTGCCCTTGCTGGAATTTCACCGATGTTCCAACCGTGCGTGGTACCGCCAAA |
| | | ACCCTGGACAGGAATTACGGGTGGTGGTTATTGGGCCAACGGACGTCGACCACTTGCATT |
| | | GGTACGAACACATTCGAAGAAAGCATTAATGCGTTACGAAGACGTATACATGCCGGAAGT |
| | | ATACAAAGCAATTAATATCGCTCAGAATACCGCATGGAAAATCAACAAAAAGGTATTGGC |
| | | AGTGGCTAACGTAATAACCAAGTGGAAACATTGTCCTGTTGAAGACATTCCTGCTATTGA |
| | | ACGAGAAGAACTACCTATGAAGCCTGAGGACATAGACATGAATCCGGAAGCATTGACCGC |
| | | GTGGAAACGAGCTGCGGCTGCTGTCTACCGAAAGGACAAGGCACGAAAATCCAGACGAAT |
| | | ATCGCTAGAATTTATGTTGGAACAAGCCAATAAGTTTGCAAACCACAAGGCGATATGGTT |
| | | TCCATACAATATGGATTGGCGAGGAAGAGTTTACGCTGTGTCTATGTTCAACCCACAGGG |
| | | AAACGACATGACAAAAGGACTATTAACATTAGCAAAAGGAAAGCCTATCGGAAAAGAAGG |
| | | TTACTATTGGTTGAAGATACACGGAGCTAATTGCGCAGGTGTCGATAAAGTCCCCTTCCC |
| | | GGAACGTATAAAATTCATAGAGGAGAACCACGAAAACATCATGGCGTGCGCCAAATCCCC |
| | | GCTAGAAAATACTTGGTGGGCGGAACAAGATTCTCCATTTTGCTTCTTAGCATTCTGCTT |
| | | TGAATACGCAGGAGTCCAGCACCACGGACTAAGTTACAATTGTTCGCTTCCTCTAGCATT |
| | | TGACGGTTCGTGCAGTGGAATACAACATTTCTCAGCCATGCTAAGAGATGAGGTGGGAGG |
| | | TCGTGCGGTAAACCTATTGCCATCGGAGACCGTGCAGGATATATATGGTATCGTCGCCAA |
| | | GAAAGTAAACGAGATTCTACAGGCGGATGCCATTAACGGAACCGACAATGAAGTTGTCAC |
| | | TGTTACCGACGAGAACACAGGTGAAATTTCAGAAAAGGTGAAGCTTGGAACTAAAGCATT |
| | | AGCCGGACAGTGGCTAGCGTATGGTGTCACTCGTAGTGTCACCAAACGTAGTGTCATGAC |
| | | GCTTGCATATGGTTCCAAAGAATTCGGTTTTAGACAGCAGGTCTTGGAGGACACGATTCA |
| | | ACCCGCCATAGATTCGGGTAAAGGATTGATGTTCACACAGCCTAATCAGGCTGCCGGTTA |
| | | TATGGCCAAACTAATTTGGGAGTCTGTTAGTGTTACAGTCGTTGCCGCTGTCGAAGCCAT |
| | | GAATTGGTTGAAAAGTGCCGCAAAACTACTTGCGGCCGAAGTGAAGGATAAGAAGACTGG |
| | | TGAGATCCTACGAAAGAGATGCGCTGTGCATTGGGTCACTCCGGATGGTTTTCCGGTCTG |
| | | GCAAGAATATAAAAAGCCGATACAAACGCGTTTGAACTTAATGTTTTTAGGACAATTCAG |
| | | ATTACAACCCACCATTAACACGAATAAAGATTCGGAAATTGACGCACACAAGCAAGAATC |
| | | AGGTATTGCACCAAACTTCGTGCACTCACAAGATGGTTCTCACTTAAGAAAAACGGTTGT |
| | | CTGGGCTCATGAGAAGTATGGTATAGAGAGTTTTGCATTGATTCATGACTCCTTCGGTAC |
| | | TATACCAGCCGACGCCGCCAATTTGTTCAAAGCGGTCCGAGAAACCATGGTAGACACTTA |
| | | CGAATCTTGCGACGTCCTTGCGGACTTTTATGATCAATTCGCTGATCAGTTACATGAGTC |
| | | GCAATTAGACAAGATGCCGGCATTACCGGCGAAGGGAAACTTGAACTTGAGAGATATTTT |
| | | AGAATCTGATTTCGCCTTTGCCTAAAAGCTTATAACTTCGTATAGCATACATTATACGAA |
| | | GTTATAATTCTGCAGATGGATCATAAAAATATAGTAGAATTTCATTTTGTTTTTTTCTAT |
| | | GCTATAAATAGCTTCTCGAGCATGCGTAAAGGCGAAGAGCTGTTCACTGGTGTCGTCCCT |
| | | ATTCTGGTGGAACTGGATGGTGATGTCAACGGTCATAAGTTTTCCGTGCGTGGCGAGGGT |
| | | GAAGGTGACGCAACTAATGGTAAACTGACGCTGAAGTTCATCTGTACTACTGGTAAACTG |
| | | CCGGTCCCTTGGCCGACTCTGGTAACGACGCTGACTTATGGTGTTCAGTGCTTTGCTCGT |
| | | TATCCGGACCATATGAAGCAGCATGACTTCTTCAAGTCCGCCATGCCGGAAGGCTATGTG |
| | | CAGGAACGCACGATTTCCTTTAAGGATGACGGCACGTACAAAACGCGTGCGGAAGTGAAA |
| | | TTTGAAGGCGATACCCTGGTAAACCGCATTGAGCTGAAAGGCATTGACTTTAAAGAAGAC |
| | | GGCAATATCCTGGGCCATAAGCTGGAATACAATTTTAACAGCCACAATGTTTAGATCACC |
| | | GCCGATAAACAAAAAAATGGCATTAAAGCGAATTTTAAAATTCGCCACAACGTGGAGGAT |
| | | GGCAGCGTGCAGCTGGCTGATCACTACCAGCAAAACACTCCAATCGGTGATGGTCCTGTT |
| | | CTGCTGCCAGACAATCACTATCTGAGCACGCAAAGCGTTCTGTCTAAAGATCCGAACGAG |
| | | AAACGCGATCATATGGTTCTGCTGGAGTTCGTAACCGCAGCGGGCATCACGCATGGTATG |
| | | GATGAACTGTACAAATAATAAATAACTTCGTATAGCATACATTATACGAAGTTATATCAA |
| | | ACTCTAATGACCACATCTTTTTTTAGAGATGAAAAATTTTCTACATCTCCTTTTGTAGAC |
| | | ACGACTAAACATTTTGCAAAAAAAAGTTTATTAGTGTTTAGATAATCGTATACTTCATCA |
| | | GTGTAGATAGTAAATGTGAACAAATAAAAGGTATTCTTACTCAATAGATTGGTAAATTCC |
| | | ATAGAATATATTAATCCTTTCTTCTTGAGATCCCACATCATTTCAACCAGAGACGTTTTA |
| | | TCCAATGATTTACCTCGTACTATACCACATACAAAACTAGATTTTGCAGTGACGTCGTAC |
| | | CTGGTATTCCTACCAAACAAAATTTTACTTTTAGTTCTTTTAGAAAATTCTAAGGTAGAA |
| | | TCTCTATTTGCCAATATGTCATCTATGGAATTACCACTAGCAAAAAATGATAGAAATATA |
| | | TATTGATACATCGCAGCTGGTTTTGATCTACTATACTTTAAAAACGAATCAGATTCCATA |
| | | ATTGCCTGTATATCATCAGCTGAAAAACTATGTTTTACACGTATTCCTTCGGCATTTCTT |
| | | TTTAATGATATATCTTGTTTAGACAATGATAAAGTTATCATGTCCATGAGAGACGCGTCT |
| | | CCGTATCGTATAAATATTTCATTAGATGTTAGACGCTTCATTAGGGGTATACTTCTATAA |
| | | GGTTTCTTAATCAGTCCATCATTGGTTGCGTCAAGAACTACTATCGGATGTTGTTGGGTA |
| | | TCTCTAGTGTTACACATGGCCTTACTAAAGTTTGGGTAAATAACTATGATATCTCTATTA |
| | | ATTATAGATGCATATATTTCATTCGTCAAGGATATTAGTATCGACTTGCTATCGTCATTA |
| | | ATACGTGTAATGTAATCATATAAATCATGCGATAGCCAAGGAAAATTCAAATAGATGTTC |
| | | ATCATATAATCGTCGCTATAATTCATATTAATACGTTGACATTGACTAATTTGTAATATA |
| | | GCCTCGCCACGAAGAAAGCTCTCGTATTCAGTTTCATC |
| 8 | T7RNA | ATGAACACAATTAACATAGCCAAGAACGACTTCAGTGACATTGAGTTGGCTGCCATCCCG |
| | polymerase | TTTAATACCTTAGCAGATCACTACGGAGAGCGACTTGCGAGAGAGCAATTGGCCCTTGAG |
| | gene | CATGAATCCTACGAAATGGGAGAAGCGCGATTCCGTAAGATGTTCGAGAGACAATTAAAA |
| | | GCCGGTGAAGTAGCGGACAACGCCGCAGCAAAGCCACTTATCACAACACTACTTCCGAAG |
| | | ATGATAGCACGTATTAACGATTGGTTTGAGGAAGTAAAGGCCAAGAGAGGTAAGAGACCG |
| | | ACGGCTTTCCAATTCCTACAAGAGATCAAGCCTGAAGCGGTGGCGTATATAACTATCAAA |
| | | ACGACCCTAGCGTGCCTAACTTCTGCCGATAACACGACGGTACAGGCTGTCGCTTCAGCG |
| | | ATTGGAAGAGCGATAGAAGATGAAGCACGTTTCGGACGAATCCGTGATCTAGAAGCTAAA |
| | | CATTTTAAAAAGAACGTTGAGGAGCAGTTGAACAAACGAGTAGGTCACGTGTACAAAAAG |
| | | GCATTCATGCAAGTGGTAGAAGCCGACATGCTATCCAAGGGATTGCTTGGAGGTGAGGCC |
| | | TGGTCGAGTTGGCACAAAGAGGACTCTATCCACGTCGGTGTTAGATGCATAGAGATGCTA |
| | | ATAGAGTCGACAGGTATGGTTTCCCTACATCGTCAAAATGCTGGAGTTGTAGGTCAGGAT |
| | | AGTGAAACGATCGAGCTAGCGCCGGAATACGCAGAGGCGATTGCGACGCGAGCTGGTGCC |
| | | CTTGCTGGAATTTCACCGATGTTCCAACCGTGCGTGGTACCGCCAAAACCCTGGACAGGA |
| | | ATTACGGGTGGTGGTTATTGGGCCAACGGACGTCGACCACTTGCATTGGTACGAACACAT |
| | | TCGAAGAAAGCATTAATGCGTTACGAAGACGTATACATGCCGGAAGTATACAAAGCAATT |
| | | AATATCGCTCAGAATACCGCATGGAAAATCAACAAAAAGGTATTGGCAGTGGCTAACGTA |
| | | ATAACCAAGTGGAAACATTGTCCTGTTGAAGACATTCCTGCTATTGAACGAGAAGAACTA |
| | | CCTATGAAGCCTGAGGACATAGACATGAATCCGGAAGCATTGACCGCGTGGAAACGAGCT |
| | | GCGGCTGCTGTCTACCGAAAGGACAAGGCACGAAAATCCAGACGAATATCGCTAGAATTT |
| | | ATGTTGGAACAAGCCAATAAGTTTGCAAACCACAAGGCGATATGGTTTCCATACAATATG |
| | | GATTGGCGAGGAAGAGTTTACGCTGTGTCTATGTTCAACCCACAGGGAAACGACATGACA |
| | | AAAGGACTATTAACATTAGCAAAAGGAAAGCCTATCGGAAAAGAAGGTTACTATTGGTTG |
| | | AAGATACACGGAGCTAATTGCGCAGGTGTCGATAAAGTCCCCTTCCCGGAACGTATAAAA |
| | | TTCATAGAGGAGAACCACGAAAACATCATGGCGTGCGCCAAATCCCCGCTAGAAAATACT |
| | | TGGTGGGCGGAACAAGATTCTCCATTTTGCTTCTTAGCATTCTGCTTTGAATACGCAGGA |
| | | GTCCAGCACCACGGACTAAGTTACAATTGTTCGCTTCCTCTAGCATTTGACGGTTCGTGC |
| | | AGTGGAATACAACATTTCTCAGCCATGCTAAGAGATGAGGTGGGAGGTCGTGCGGTAAAC |
| | | CTATTGCCATCGGAGACCGTGCAGGATATATATGGTATCGTCGCCAAGAAAGTAAACGAG |
| | | ATTCTACAGGCGGATGCCATTAACGGAACCGACAATGAAGTTGTCACTGTTACCGACGAG |
| | | AACACAGGTGAAATTTCAGAAAAGGTGAAGCTTGGAACTAAAGCATTAGCCGGACAGTGG |
| | | CTAGCGTATGGTGTCACTCGTAGTGTCACCAAACGTAGTGTCATGACGCTTGCATATGGT |
| | | TCCAAAGAATTCGGTTTTAGACAGCAGGTCTTGGAGGACACGATTCAACCCGCCATAGAT |
| | | TCGGGTAAAGGATTGATGTTCACACAGCCTAATCAGGCTGCCGGTTATATGGCCAAACTA |
| | | ATTTGGGAGTCTGTTAGTGTTACAGTCGTTGCCGCTGTCGAAGCCATGAATTGGTTGAAA |
| | | AGTGCCGCAAAACTACTTGCGGCCGAAGTGAAGGATAAGAAGACTGGTGAGATCCTACGA |
| | | AAGAGATGCGCTGTGCATTGGGTCACTCCGGATGGTTTTCCGGTCTGGCAAGAATATAAA |
| | | AAGCCGATACAAACGCGTTTGAACTTAATGTTTTTAGGACAATTCAGATTACAACCCACC |
| | | ATTAACACGAATAAAGATTCGGAAATTGACGCACACAAGCAAGAATCAGGTATTGCACCA |
| | | AACTTCGTGCACTCACAAGATGGTTCTCACTTAAGAAAAACGGTTGTCTGGGCTCATGAG |
| | | AAGTATGGTATAGAGAGTTTTGCATTGATTCATGACTCCTTCGGTACTATACCAGCCGAC |
| | | GCCGCCAATTTGTTCAAAGCGGTCCGAGAAACCATGGTAGACACTTACGAATCTTGCGAC |
| | | GTCCTTGCGGACTTTTATGATCAATTCGCTGATCAGTTACATGAGTCGCAATTAGACAAG |
| | | ATGCCGGCATTACCGGCGAAGGGAAACTTGAACTTGAGAGATATTTTAGAATCTGATTTC |
| | | GCCTTTGCCTAA |

1. A virus comprising a retroviral envelope construct, a retroviral packing construct, and a retroviral transfer construct, wherein at least one of the envelope construct, the packaging construct, or the transfer construct comprises a transgene that codes for a non-retroviral protein, and wherein the virus is an oncolytic virus.

2. The virus of claim 1, wherein the envelope construct, the packing construct, and the transfer construct are inserted at different locations within a genome of the oncolytic virus.

3. The virus of claim 1, wherein the envelope construct comprises a gene that codes for a retroviral envelope protein and the packaging construct comprises a gene that codes for a retroviral structural protein.

4. The virus of claim 3, wherein the gene that codes for the retroviral envelope protein is under the control of a first early viral promoter and the gene that codes for the retroviral structural protein is under the control of a second early viral promoter.

5. The virus of claim 3, wherein the gene that codes for the retroviral envelope protein is under the control of a first early viral promoter, a first early/late viral promoter or a first late viral promoter; and the gene that codes for a retroviral structural protein is under the control of a second early viral promoter, a second early/late viral promoter, or a second late viral promoter.

6. The virus of claim 1, wherein the oncolytic virus is a measles virus, a poliovirus, a poxvirus, a vaccinia virus, an adenovirus, an adeno-associated virus, a herpes simplex virus, a vesicular stomatitis virus, a reovirus, a Newcastle disease virus, a senecavirus, a mengovirus, or a myxomavirus.

7. The virus of claim 6, wherein the oncolytic virus is a vaccinia virus.

8. The virus of claim 7, wherein the vaccinia virus comprises a mutation or deletion of at least one viral gene.

9. The virus of claim 8, wherein the at least one viral gene is selected from the group consisting of: B5R, A52R (VACWR178), F13L, A36R, A34R, A33R, B8R, B18R, SPI-1, SPI-2, B15R, VGF, E3L, K3L, A41L, K7R, NIL, C12L, TK, and any combinations thereof.

10. The virus of claim 1, wherein the non-retroviral protein comprises a therapeutic protein or a diagnostic protein.

11. The virus of claim 10, wherein the non-retroviral protein comprises the therapeutic protein, and wherein the therapeutic protein comprises an immune checkpoint modulator, an antibody or portion thereof, a Fc fusion protein, an anticoagulant, a blood factor, a bone morphogenetic protein, an immunosuppressive agent, an immunostimulatory agent, an enzyme, a growth factor, a hormone, an interferon, an interleukin, a thrombolytic, an anti-angiogenic, a chemotherapeutic, an antibiotic, an antifungal, an antiviral, and any combination thereof.

12. The virus of claim 1, wherein the retroviral envelope construct, the retroviral packing construct, and the retroviral transfer construct are from an Alpharetrovirus, a Betaretrovirus, a Deltaretrovirus, an Epsilonretrovirus, a Gamma retrovirus, a Lentivirus, or any combination thereof.

13. The virus of claim 12, wherein the retrovirus is the lentivirus.

14. The virus of claim 13, wherein the lentivirus is an HIV.

15. The virus of claim 12, wherein the retrovirus is the Gamma retrovirus.

16. The virus of claim 15, wherein the Gamma retrovirus is a Moloney murine leukemia virus.

17. The virus of claim 1, comprising an exogenous nucleic acid sequence that codes for a nucleic acid polymerase.

18. The virus of claim 17, wherein the nucleic acid polymerase is selected from the group consisting of: T7 RNA polymerase, T3 RNA polymerase and SP6 RNA polymerase, a RNA polymerase variant, and a DNA polymerase mutant.

19. The virus of claim 1, comprising an exogenous nucleic acid sequence that codes for a bacteriophage polymerase.

20. The virus of claim 19, wherein the bacteriophage is selected from the group consisting of a T3 bacteriophage, a T7 bacteriophage, and an SP6 bacteriophage.

21. The virus of claim 20, wherein a T3 bacteriophage polymerase is expressed with a T3 bacteriophage promoter, a T7 bacteriophage polymerase is expressed with a T7 bacteriophage promoter, and an SP6 bacteriophage polymerase is expressed with an SP6 bacteriophage promoter.


SEQUENCE LISTING (14 sequences)

SEQ ID NO: 1 - DNA, 194711 bp, Vaccinia virus
atttaaaatataatattaatgtactaaaacttatatattattaatttatctaactaaagttagtaaattatatatataattttataattaatttaattttactaattttatttagtgtctagaaaaaaatgtgtgacccatgactgtaggaaactctagagtgtaagaaagatcgatcgctttatagagaccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaaggagaaagagataaaacttttttacgactccatcagaaagaggtttaatatttttgtgagaccatcgaaggagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaaggagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagaaagagatagttgatctagatatttttcttagtacaaaagtcaatgttttaaaatatatggacaagaatttgtctgtataaaaacttgtgtgaaattttgtaccaaagaaaaaatgtgagcagtatcccctacatggattttactagatcatttatataccaaaaaatattatacgatctacgttttattatatgattttaacgtgtaaattataaacattattttatgatatacaattgtctggtaacctagatgggcataggggatgttgataagctcgacgagtatatgttgttggacgttattgtttaagaaatagttgatgcatcagaaagagaataaaaaatattttagtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagaataaaaatattttatgactccattgaagagagaaagagaaaatgagaatgagaataaaaatattttagtgacaccatcagaaagaggtttaatatttttgtgagaccatcgaagagagaaagagaataaaaatattttatgactccattgaagagagaaagagaaaatgagaatgagaataaaaatattttagtgacaccatcagaaagaggtttaatattttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcagaaagaggtttaatatttttgtgataccctgaaaggaaataggaataggaataggaatagtgtcataatcgtatcacactattgagacagaaaaagaagaagtcgcgagaggtaactttttgtgaatgtagttaagaacatttttgttttgcaaaccggaatatagtgtccggtacacttttttaattcgtggtgtgcctgaatcgttcgattaaccctactcatccaatttcagatgaatagagttatcgattcagacacacgctttgagttttgttgaatcgatgagtgaagtatcatcggttgcaccttcagatgccgatccgtcgacatacttaaatccatccttgacctcaagttcagatgattccttgcacatgtctccgatacgaacgctaaactctagattcttgacacattttgtatcgacgatcgttgaaccgatgatatcttcgtaactcactttcttatgagagatgttagacccgagtactggatgggtcttgatgtcgctgtctttctcttcttcgctacatctgatgtcgatagacacctcacagtctttgatcatagccagagcttcttcatgagtgatcgcgggagagtccttaccttgtcctggggacacgctggacaatctagcattcactgtgtttccatcagcggattctgagatggatttaatctgaggacatttggtgaatccaaagttcattctcagacctccaccgatgatggagtaataagtggtaggaggatctacatcctcgactgatgtggaatcatcttctgattccacctcgggatctggatctgactcggactctgtaatttccgttacggattggcaaatcttatcattggtcggtgtttggtcttgctttgtgactttgataataacatcgattcccatatgatgtttgttttcttcttccgtacacgaggaggaggatgaggatgattgctgaagactggcaggcacatgcatgccaggacgatatattgtttcatgattgctattgattgagtactgttctttatgattctacttccttaccgtgcaataaattagaatatattttctacttttacgagaaattaattattgtatttattatttatgggtgaaaaacttactataaaaagcgggtgggtttggaattagtgatcagtttatgtatatcgcaactaccgggcatatggctatcgacatcgagaacattacccacatgataagagattgtatcagtttcgtagtcttgagtattggtattactatatagtatatagatgtcgacgctagatagacagtctccgaatgcggcatgataccgtcatcattctttgctttcgttaactgtttggaggaaaaatttttgttattgcatttaatctcgaaattcagagtgcacacctttctcctgtaaagaaacctgaagttgctaccttattaaggacggagaagtattcctcacgaaatacgggattacagtctttatgattcatagtaatagttagttccgacgttgagatggattcgctgagaccggtagtggtcgttaactggatacagattaatttccacatcgatatagttaaaggtattactgggtacgggttcgcatttatctgcggaagagacggtgtgagaatatgttccgagaccacacggagaacagatgacgtctccggatactccgtatcctattccacattttgtttgggaaacacatgccttgcatccggatgatcctttgagaagacaataatatccgggagagcattcacagattctattgtgagtcgtgttacacggtcgcgtcttccgttacaacttagacaagcgggtaaatgattattgcgagatgtgaaggtacccgaaccacacggcgtacattgtgtgttagtcttgctatcgcataatctggaagcgtatgttcccggacacaaattatggcgtttgtattcgttgtctttacactttccatcggatggtgcatgcggtgctatatctcttccgtttattattatacatgagagaaacaatatatacgagtataatacggacttcatgatttaataatgtagtaatcgtcgtcttgttcctgtttcctacttctccaatcatatagatattttctttctatcatggataatatttgtaatggttcttttcgtacaacatactgtttagatgatattgcgcataatttccggaggcaaatacgatagtctagattgaccgatggtagactctaatttattgagtgctttgtcgacgagtttacttttacgctccatcgatagatggcactgttctatgagatcgtcgtacatgggaaatgaaatgtgactgtctgaatgtatggctttaagatagctgtgataccgtatacaggtcggtgtcggagattcgaatctctttaaggcgacttatgtcacgatgatggaatctatcttatcgaatgatatatttttcataaatacacttttatagtcctcgtttaaacagaatttactatgtagttccgcgaatgactcgtcccttaataggcagtaggctattatcttctttacgtagtaatcgtcgtagggagagacatcttgtagaacaacgatttaatcataggtagagatactttcagtctgtggtggatgatgtcattcacaacatccgccttgtatatgatgtttctgttttcaaacaccaagtcgaataccgtctttagtcggaaggttgatgtcgtatccgatgtatgaggcaacattgttgttacaattttgaaaggcggtattatagtattcgtctttctgaatgtcgaacctatctagtagataccgtagtatattgagagtgtatccttgattatgttttatgaatagataaagtagatgttgtccttcttccttttgttcgtgccaattgagtaacattatgagaatatgacctgttgcacaatcgttccatgatgggtgtacaatcaagattattacgtatcctcgtatcggctcctcgagataaaagagcatacaccacacgaggactatgtttggtatactgttgaaggtaagtgtgtaaccgcgttaatgtttgctccataatctattatcgcgtagatgaatcgcttctcggctcgcatcttagtgtgacttaacttgtaataattgcttttgtagaacgtggatatgtgtttacagtagtaatgaagagaagtgagttcatcctcgtcggatcctttgtacagaacgtaatagtttaagctcccattgaatttatatctaagataacacagcaatagatcggatgatttactaaagtcatcaatggtgtccgttagtatatcaaagatcttgttatcgattgatagtggtgtcctttttcatccttgctatcaaagttacgcatgccgtggtgtaacaatatctttaatacagatggattaaatcgtgtattcatcgtatagcaatgtaatggagagttacctcgtttattcagatcgcagtgtttaataactagcttaaacagatgagacgatgtatccacatcaaagaacgtgaaatacatatgacagacattgttgacagaaacgtgaccttcattcttaccgtcgtccataaatacgttaggtatgtaccacatactgtcgcgaacgatgcgtacaatctcgtccatctcataatgatttactttttcataattaaagatgtgaaagaaaaacagaacaatatatttttttagtaatgtttatgcgagacatataaaataaactccgtgtttatgatcatttttaacagcaacacattcaatattgtattgttatttttatattatttacacaattaacaatatattattagtttatattactgaattaataatataaaattcccaatcttgtcataaacacacactgagaaacagcataaacacaaaatccatcaaaaatgtcgatgaaatatctgatgttgttgttcgctgctatgataatcagatcattcgccgatagtggtaacgctatcgaaacgacatcgccagaaattacaaacgctacaacagatattccagctatcagattatgcggtccagagggagatggatattgtttacacggtgactgtatccacgctagagatattgacggtatgtattgtagatgctctcatggttatacaggcattagatgtcagcatgtagtattagtagactatcaacgttcagaaaacccaaacactacaacgtcatatatcccatctcccggtattatgcttgtattagtaggcattattattattacgtgttgtctattatctgtttataggttcactcgacgaactaaactacctatacaagatatggttgtgccataatttttataaatttttttatgagtatttttacaaaaaaaatgtataaagtgtatgtcttatgtatatttataaaaatgctaagtatgcgatgtatctatgttatttgtatttatctaaacaatacctctacctctagatattatacaaaaattttttatttcggcatattaaagtaaaatctagttaccttgaaaatgaatacagtgggtggttccgtatcaccagtaagaacataatagtcgaatacagtatccgattgagattttgcatacaatactagtctagaaagaaatttgtaatcatcttctgtgacgggagtccatatatctgtatcatcgtctagtttatcagtgtcccatgctatattcctgttatcatcattagttaatgaaaataactctcgtgcttcagaaaagtcaaatattgtatccatacatacatctccaaaactatcgcttatacgtttatctttaacgatacctatacctagatggttatttactaacagacattttccagatctattgactataactcctatagtttccacatcaaccaagtaatgatcatctattgttatataacaataacataactcttttccatttttatcagtatgtatatctatatcaacgtcgtcgttgtagtgaatagtagtcattgatctattatatgaaacggatatgtctagaacggcaattgttttacgtccagttaacactttctttgatttaaagtctagagtctttgcaaacataatatccttatccgactttatatttcctgtagggtggtataattttattttgcctccacatatcggtgtttccaaatatattactagacaatattccatatagttattagttaagggtacccaattagaacacgtacgcttattatcatcatttggatcgtatttcataaaagttattgtactatcgatgtcaacacattctacattttttaatcgtctatatagtatttttctgatattttctataatatcagaattgtcttccatcggaagttgtatactatcggaatcagttacatgtttaaataattctctgatgtcattccttatacaatcaaattcattattaaacagtttaatagtctgtagacctttatcgtcgtaaatatccattgtcttattagttacgcttatttttatgtgttttacgttgctttattatattttataagaatgattgtttgacgaatcacgagaactattaagacacattattaggtatatattataaaaaagtttttgattacgatgttataagaggaaagaggacacattaacatcatacatcaattaactacattcttataacatcgtaatcaaaagaattgcaattttgatgtataacaactgtcaatgggttatggaattgtatattacatattatacggtatgttggtaacgacaaataccgatcggtaattgtctgccggtgtaatagaattatatatatctatctattacaccggctgagtatgcataataataagttgtggtagtatgatctccatatttataatttaggactttgtattcagtatttttggaatcataaaaaataaaaaaaagttttactaatttaaaatttaaaaagtatttacatttttttcactgtttagtcgcggatatggaattcgatcctgccaaaatcaatacatcatctatagatcatgtaacaatattacaatacatagatgaaccaaatgatataagactaacagtatgcattatccgaaatattaataacattacatattatatcaatatcacaaaaataaatacacatttggctaatcaatttcgggcttggaaaaaacgtatcgccggaagggactatatgactaacttatctagagatacaggaatacaacaatcaaaacttactgaaactatacgtaactgtcaaaaaaatagaaacatatatggtctatatatacactacaatttagttattaatgtggttattgattggataaccgatgtgattgttcaatcaatattaagagggttggtaaattggtacatagctaataatacctatacacccaataatacaacaaccatttctgagttggatatcatcaaaatactggataaatacgaggacgtgtatagagtaagtaaagaaaaagaatgtggaatttgctatgaagttgtttactcaaaacgatagatactttggtttattggattcgtgtactcatatattttgcataacatgcatcaatatatggcataaaacacgaagagaaaccggtgcgtcggataattgtcctatatgtcgtacccgttttagaaacataacaatgagcaagttctataagctagttaactaataaataaaaagtttaatttgttgacgacgtatgtcgttatttttctcgtatgaaagattaaattcaattcaattcgttgtttctaatataatctgccgtattggatggattctcaagacaattgcatttagattatattatcatgaataaaaatagtagcacgcactacttcagccaaatattcttttttgaaacgccatctatcgtagtgaggacacaagtgaacctataattatcaaatttattagtatcagtcacatgaaggactttctgtagagtgacgattctaccatctatggtactaacggtttcatcctccttgataccctcacccaaatgttctataaatttagcatcctcgtccgatctcatatcctttgccaaccaatacatgtagctaaaattaggcataaatttcacacatccagtgcaacgaaattctccagaagatgttacgatgtttaggttaggacatttgatttcgtcggcattaacatatgggtgaacacacccatacatgaaagcgatgagaaataggattctcatcttgccaaaatatcactagaaaaaatttatttatcaattttaaaggtataaaaaatacttattgttgctcgaatattttgtatttgatggtatacggaagattagaaatgtaggtattatcatcaactgattctatggttttatgtattctatcatgtttcactattgcgtcggaaataatatcatatgcttccacatatattttattttgttttaactcataatactcacgtaattctggattattggcatatctatgaataattttagctccatgatcagtaaatattaatgagaacatagtattaccacctaccattatttttttcatttcgttcaattcttgattgcaaagatctatataatcattatagcgttgacttatggactctggaatcttagacgatgtacagtcatctataatcatggcatatttaatacattgttttatagcatagtagttatctacgatgttagatatttctctcaatgaatcaatcacacaatctaatgtaggtttatgacataatagcattttcagcagttcaatgtttctagattcgttgatggcaatggctatacatgtatatccgttatttgatctaatgttgacatctgaaccggattctagcagtaaagatactagagattgtttattatatctaacagccttgtgaagaagtgtttctcctcgtttgtcaatcatgttaatgtctttaagataaggtaggcaaatgtttatagtactaagaattgggcaagcataagacatgtcacaaagaccctttttgtatgtataagtgtaaaaattataacattcatagttggatttacataggtgtccaatcgggatctctccatcatcgagataattgatggcatctcccttccttttttagtagatatttcatcgtgtaagaatcaatattaatatttctaaagtattcgtgtatagcctctttatttaccacagttccatattccactagagggatatcgccgaatgtcatatactcaattagtatatgttggaggacatccgagttcattgttttcaatatcaaaaagatggtttccttatcatttctccatagtggtacaatactacacattattccgtgcggctttccattttccaaaaacaatttgaccaaatctaaatctacatctttattgtatctataatcactatttagataatcagccataattactcgagtgcaacatgttagatcgtctatatatgaataagcagtgttatctattcctttcattaacaatttaacgatgtctatatctatatgagatgacttaatataatattgaagagctgtacaatagtttttatctatagaagacggcttgattccgtgattaattagacatttaacaacttccggacgcacatatgctctcgtatccgactttgaatacagatgagagatgatatacagatgcaatacggtaccgcaatttcgtagttgataatcatcatacgcgtatcagtactcgtcctcataaagaacactgcagccattttctatgaacaaatcaataattttaggaacaggatcattgtcattacataattttctataactgaacgatggttttcacatttaacactcaagtcaaatccatgttctaccaacacctttatcaagtcaacgtctacatttttggatttcatatagctgaatatattaaagtcatttatgttgctaaatccagtggcttctagtagagccatcgctatatcctttaactttaacatgtctactatttgtgtattcttctaatggggtagctgtctccaatttttgcgtaatggattagtgccactgtctagtagtagtttgacgacctcgacattattacaatgctcattaaaaaggtatgcgtgtaaagcattattcttgaattggttcctggtatcattaggatctctgtctctcaacatctgtttaagttcatcgagagccacctcctcattttccagatagtcaaacattttgactgaatgagctactgtgaactctatacacccacacaactaatgtcattaaatattatttttttgaatgtatttataccatgtcaaaaacttgtacaattattaataaaaataatttagtgtttaaattttaccagttccagattttacacctccgttaaccccactttttacaccactggacgatcctcctccccacattccaccgccaccagatgtataagttttagatcctttattactaccatcatgtccatggataaagacactccacatgccgccactactaccccctttagaagacatattaataagacttaaggacaagtttaacaataaaattaatcacgagtaccctactaccaacctacactattatatgattatagtttctatttttacagtaccttaactaaagtctctagtcacaagagcaatactaccaacctacactattatatgattatagtttctatttttataggaacgcgtacgagaaaatcaaatgtctaatttctaacggtagtgttgataaacgattatcgtcaatggatacctcctctatcatgtcgtctattttcttactttgttctattaacttattagcattatatattatttgattataaaacttatattgcttattagcccaatctgtaaatatcggattattaacatatcgtttctttgtaggtttatttaacatgtacatcactgtaagcatgtccgtaccatttattttaatttgacgcatatccgcaatttctttttcgcagtcggttataaattctatatatgatggatacatgctacatgtgtacttataatcgactaatatgaagtacttgatacatattttcagtaacgatttattattaccacctatgaataagtacctgtgatcgtctaggtaatcaactgttttcttaatacattcgatggttggtaatttactcagaataatttccaatatcttaatatataattctgctatttctgggatatatttatctgccagtataacacaaatagtaatacatgtaaacccatattttgttattatattaatgtctgcgccattatctattaaccattctactaggctgacactatgcgacttaatacaatgataaagtatactacatccatgtttatctattttgtttatatcatcaatatacggcttacaaagttttagtatcgataacacatccaactcacgcatagagaaggtagggaataatggcataatatttattaggttatcatcattgtcattatctacaactaagtttccattttttaaaatatactcgacaactttaggatctctattgccaaatttttgaaaatatttatttatatgcttaaatctatataatgtagctccttcatcaatcatacatttaataacattgatgtatactgtatgataagatacatattctaacaatagatcttgtatagaatctgtatatcttttaagaattgtggatattaggatattattacgtaaactattacacaattctaaaatataaaacgtatcacggtcgaataatagttgatcaactatataattatcgattttgtgatttttcttcctaaactgtttacgtaaatagttagatagaatattcattagttcatgaccactatagttactatcgaataacgcgtcaaatatttcccgtttaatatcgcatttgtcaagataataatagagtgtggtatgttcacgataagtataataacgcatctcttttttgtgtgaaattaaatagtttatcacgtccaaagatgtagcataaccatcttgtgacctagtaataatataataatagagaactgttttacccattctatcatcataatcagtggtgtagtcgtaatcgtaatcgtctaattcatcatcccaattataatattcaccagcacgtctaatctgttctattttgatcttgtatccatactgtatgttgctacatgtaggtattcctttatccaataatagtttaaacacatctacattgggatttgatgttgtagcgtatttctctacaatattaataccatttttgatactatttatttctatacctttcgaaattagtaatttcaataagtctatatcgatgttatcagaacatagatattcgaatatatcaaaatcattgatatttttatagtcgactgacgacaataacaaaatcacaacatcgtttttgatattattatttttcttggtaacgtatgcctttaatggagtttcaccatcatactcatataatggatttgcaccactttctatcaatgattgtgcactgctggcatcgatgttaaatgttttacaactatcatagagtatcttatcgttaaccatgattggttgttgatgctatcgcattttttggtttctttcatttcagttatgtatggatttagcacgtttgggaagcatgagctcatatgatttcagtactgtagtgtcagtactattagtttcgatcagatcaatgtctagatctatagaatcaaaacacgataggtcagaagataatgaatatctgtacgcttctttttgtactgtaacttctggttttgttagatggttgcatcgtgctttaacatcaatggtacaaattttatcctcgctttgtgtatcatattcgtctctagtataaaattctatattcagattatcatgcgatgtgtatacgctaacggtatcaataaacggagcacaccatttagtcataacagtaatccaaaattttttaaagtatatcttaacgaaagaagttgtgtcattgtctacggtgtatggtactagatcctcataagtgtatatatctagagtaatgtttaatttattaaatggttgataatatggatcctcatgacaatttccgaagatggaaatgagatatagacatgcaataaatctaatcgaagacatggttactccttaaaaaaatacgaataatcaccttggctatttagtaagtgtcatttaacactatactcatattaatccatggactcataatctctatacgggattaacggatgttctatatacggggatgagtagttttcttctttaactttatactttttactaatcatatttagactgatgtatgggtaatagtgtttaaagagttcgttctcatcatcagaataaatcaatatctctgtttttttgttatacagatgtattacagcctcatatattacgtaatagaacgtgtcatctaccttattaactttcaccgcatagttgtttgcaaatacggttaatcctttgacctcgtcgatttccgaccaatctgggcgtataatgaatctaaactttaatttcttgtaatcattcgaaataatttttagtttgcatccgtagttatcccctttatgtaactgtaaatttctcaacgcgatatctccattaataatgatgtcgaattcgtgctgtatacccatactgaatggatgaactaacgaatatcaacggcgttaatagtaatttactttttcatctttacatattgggtactagttttactatcataagtttataaattccacaagctactatggaataagccaaccatcttagtataccacacatgtcttaaagtttattaattaattacatgttgttttatatatatcgctacgaatttaaagagaaatcagtttaggaagaaaaaaattatctatctacatcatcacgtctctgtattctacgatagagtgctactttaagatgagacatatccgtgtcatcaaaaatatactccattaaaatgattattccggcagcgaacttgatattggatatatcacaacctttgttaatatctacgacaatagacagcagtcccatggttccataaacagtgagtttatctttctttgaagcgatagtttgtagagatcttataaaaccgtcaaacgacatcgcatttatatctttagctaattcatatatgttaccatcgtaatatctaaccgcgtctatcttaaacgtttccatcgctttaaagacgtttccgatagatggtctcatttcatcagtcatactgagccaacaaatataatcgtgtataacatctttgatagaatcagactctaaagaaaacgaatcggctttattatacgcattcatgataaacttaatgaaaaatgtttttcgttgtttaagttggatgaatagtatgtcttaataattgttattatttcattaattaatatttagtaacgagtacactctataaaaacgagaatgacataactagttatcaaagtgtctaggacgcgtaattttcatatggtatagatcctgtaagcattgtctgtattctggagctattttctttatcgcattagtaagttcagaatatgttataaatttaaatcgaataacgaacatgactttagtaaagtcgtctatattaactcttttattttctagccatcgtaataccatgtttaagatagtatattctctagttactacgatctcatcgttgtctagaatatcacatactgaatctacatccaattttagaaattggtctgtgttacatatctcttctatattattgttgatgtattgtcgtagaaaactattacgtagaccattttctttataaaacgaatatatagtactccaattatctttaccgatatatttgcacacataatccattctctcaatcactacatctttaagattttcgttgttaagatatttggctaaactatataattctattagatcatcaacagaatcagtatatatttttctagatccaaagacgaactctttggcgtcctctataatattcccagaaaagatattttcgtgttttagtttatcgagatctgatctgttcatatacgccatgattgtacggtacgttatgataaccgcataaaataaaaatccattttcatttttaaccaatactattcataattgagattgatgtaatactttgttactttgaacgtaaagacagtacacggatccgtatctccaacaagcacgtagtaatcaaatttggtgttgttaaacttcgcaatattcatcaatttagatagaaacttatactcatcatctgttttaggaatccatgtattattaccactttccaacttatcattatcccaggctatgtttcgtccatcatcgttgcgcagagtgaataattcttttgtattcggtagttcaaatatatgatccatgcatagatcggcaaagctattgtagatgtgatttttcctaaatctaatataaaactcgtttactagcaaacactttcctgatttatcgaccaagacacatatggtttctaaatctatcaagtggtggggatccatagttatgacgcagtaacatagattattacattcttgactgtcgctaatatctaaatatttattgttatcgtattggattctgcatatagatggcttgtatgtcaaagatatagaacacataaccaatttatagtcgcgctttacattctcgaatctaaagttaagagatttagaaaacattatatcctcggatgatgttatcactgtttctggagtaggatatattaaagtctttacagatttcgtccgattcaaataaatcactaaataatatcccacattatcatctgttagagtagtatcattaaatctattatattttatgaaagatatatcactgctcacctctatatttcgtacatttttaaactgtttgtataatatctctctgatacaatcagatatatctattgtgtcggtagacgataccgttacatttgaattaatggtgttccattttacaacttttaacaagttgaccaattcatttctaatagtatcaaactctccatgattaaatattttaatagtatccattttatatcactacggacacaaagtagctgacataaaccattgtataatttttatgttttatgtttattagcgtacacattttggaagttccggcttccatgtatttcctggagagcaagtagatgatgaggaaccagatagtttatatccgtacttgcacttaaagtctacattgtcgttgtatgagtatgatcttttaaacccgctagacaagtatccgtttgatattgtaggatgtggacatttaacaatctgacacgtgggtggatcggaccattctcctcctgaacacaggacaccagagttaccaatcaacgaatatccactattgcaactataagttacaacgctcccatcggtataaaaatcctcgtatccgttatgtcttccgttggatatagatggaggggattggcatttaacagattcacaaataggtgcctcgggattccataccatagatccagtagatcctaattcacaatacgatttagattcaccgatcaaatgatatccgctattacaagagtacgttatactagagccaaagtctactccaccaatatcaagttggccattatcgatatctcgaggcgatgggcatctccgtttaatacattgattaaagagtgtccatccagtacctgtacatttagcatatataggtcccattttttgctttctgtatccaggtagacatagatattctatagtgtctcctatgttgtaattagcattagcatcagtctccacactattcttaaatttcatattaatgggtcgtgacggaatagtacagcatgatagaacgcatcctattcccaacaatgtcaggaacgtcacgctctccaccttcatatttatttatccgtaaaaatgttatcctggacatcgtacaaataataaaaagcccatatatgttcgctattgtagaaattgtttttcacagttgctcaaaaacgatggcagtgacttatgagttacgttacactttggagtctcatctttagtaaacatatcataatattcgatattacgagttgacatatcgaacaaattccaagtatttgattttggataatattcgtattttgcatctgctataattaagatataatcaccgcaagaacacacgaacatctttcctacatggttaaagtacatgtataattctatccatttgtcttccttaactatatatttgtatagataattacgagtctcgtgagtaattccagtaattacatagatgtcgccgtcgtactctacagcataaactatactatgatgtctaggcatgggagacttttttatccaacgatttttagtgaaacattccacatcgtttaatactacatatttttcatacgtggtataaactccacccattacatatatatcatcgtttacgaataccgacgcgcctgaatatctaggagtaattaagtttggaagtcttatccatttcgaagtgccgtgtttcaaatattctgccacacccgttgaaatagaaaattctaatcctcctattacatataactttccatcgttaacacaagtactaacttctgattttaacgacgacatattagtaaccgttttccattttttcgtttcaagatctacccgcgatacggaataaacatgtctattgttaatcatgccgccaataatgtatagacaattatgtaaaacatttgcattatagaattgtctatctgtattaccgactatcgtccaatattctgttctaggagagtaatgggttattgtggatatataatcagagtttttaatgactactatattatgttttataccatttcgtgtcactggctttgtagatttggatatagttaatcccaacaatgatatagcattgcgcatagtattagtcataaacttgggatgtaaaatgttgatgatatctacatcgtttggatttttatgtatccactttaataatatcatagctgtaacatcctcatgatttacgttaacgttttcgtgggataagatagttgtcagttcatcctttgataattttccaaattctggatcggatgtcaccgcagtaatattgttgattatttctgacatcgacgcattatatagttttttaattccatatcttttagaaaagttaaacatccttatacaatttgtggaattaatattatgaatcatagtttttacacatagatctactacaggcggaacatcaattattatggcagcaactagtatcatttctacattgtttatggtgatgtttatcttcttccagcgtatatagtctaatagcgattcaaacgcgtgatagtttataccattcaatataatcgcttcatcctttagatggtgatcctgaattcgtttaaaaaaattatacggagatgccgtaataatttccttattcacttgtataatttccccattgatagaaaatatcacgctttccattcttgaagtactataagtaattatagtataatgtaaaggtttatatattcaatattttttataaaaaaatcattttgacattaattcctttttaaatttccgtctatcatctatagaaacgtattctatgaatttataaaatgcttttacgtgtcctatcgtaggcgatagaaccgctaaaaagcctatcgaatttctacaaaagaatctgttatatggtatagggagagtataaaacattaaatgtccgtacttattaaagtattcagtagccaatcctaactctttcgaatacttattaatggctcttgttctgtacgaatctatttttttgaacaacggacctagtggtatatcttgttctatgtatctaaaataatgtctgactagatccgttagtttaatatcctcagtcatcttgtctagaatggcaaatctaactgcgggtttaggctttagtttagtttttatatctacatctatgtctttatctaacaccaaaaatataatagctaatattttattacaatcatccggatattcttctacgatctcactaactaatgtttctttggttatactagtatagtcactatcggacaaataaagaaaatcagatgatcgatgaataatacatttaaattcatcatctgtaagatttttgagatgtctcattaaaatattattagggtcagtactcattatcattcggcagctattacttattttatttttctgtattttattatttttcaccatatagatcaatcattagatcatcaaaatatgtttcaatcatcctaaagagtatggtgaatgactcttcccatctaatttctgaacgttcaccaatgtctctagccactttggcactaatagcgatcattcgcttagcgtcttctatattattaactggttgattcaatctatctagcaatggaccgtcggacagcgtcattctcatgttcttaatcaatgtacatacatcgccgtcatctaccaattcatccaacaacataagctttttaaaatcatcattataataggtttgatcgttgtcatttctccaaagaatatatctaataagtagagtcctcatgcttagtaatttaactattttagttaacaactattttttatgttaaatcaattagtacaccgctatgtttaatacttattcatattttagtttttaggattgagaatcaatacaaaaaattaatgcatcattaattttagaaatacttagtttccacgtagttaatgaaacatttgaactcatcgtacaggacgttctcgtacaggacgtaactataaaccggtttatatttgttcaagatagatacaaatccgataactttttttacgaattctacgggatccactttaaaagtgtcataccgggttctttttattcttttaaacagatcaatggtgtgatgttgattaggtcttttacgaatttgatatagaatagcgttcacatatcctccataatggtcaatcgccatttgttcgtatgtcataaattctttaattatatgacactgtgtattatttagttcatccttgttcatcattaggaatctatccaaaatggcaattatactagaactataggtgcgttgtatacacatattgatgtgtctgtttatacaatccatgatatttggatccatgctactaccttcgggtaaaattgtagcatcatataccatttctagtactttaggttcattattatccattgcagaggacgtcatgatcgaatcctaaaaaaatatattatttttatgttattttgttaaaaataatcatcgaatacttcgtaagatactccttcatgaacataatcagttacaaaacgtttatatgaagtaaagtatctacgatttttacaaaagtccggatgcataagtacaaagtacgcgataaacggaataataatagatttatctagtctatctttttctatagctttcatagttagatacatggtctcagaagtaggattatgtaacatcagcttcgataaaatgactgggttatttagtcttacacattcgctcatacatgtatgaccgttaactacaaagtctacactaaaatgattgaacaatagatagtctaccattgtttcgtattcagatagtacagcgtagtacatagcatcttcacaaattatatcattgtctaatagatatttgacgcatcttatggatcccacttcaacagccatcttaaaatcggtagaatcatattgctttcctttatcattaataatttctaaaacatcatctctatcataaaagatacaaatattaactgtttgatccgtaataacattgctagtcgatagcaatttgttaataagatgcgctgggctcaatgtcttaataagaagtgtaagaggactatctccgaatttgttttgtttattaacatccgttgatggaagtaaaagatctataatgtctacattcttgactgttttagagcatacaatatggagaggtgtatttccatcatgatctggttttgagggactaattcctagtttcatcatccatgagattgtagaagcttttggattgtctgacataagatgtctatgaatatgatttttgccaaatttatccactatcctggcttcgaatccgatggacattatttttttaaacactctttctgaaggatctgtacacgccaacaacggaccacatccttcttcatcaaccgagttgttaatcttggctccatactgtaccaataaatttattctctctatgacttcatcatctgttcccgagagataatatagaggtgttttattatgtttatcacacgcgtttggatctgcgccgtgcgtcagcagcatcgcgactattctattattattaattttagaagctatatgcaatggataatttccatcatcatccgtctcatttggagagtatcctctatgaagaagttcttcgacaaatcgttcatctagtcctttaattccacaatacgcatgtagaatgtgataattatttccagaaggttcgatagcttgtagcatattcctaaatacatctaaatttttactattatatttggcataaagagatagataatactcggccgacataatgttgtccattgtagtataaaaattaatatttctatttctatttctgtatatttgcaacaatttactctctataacaaatatcataacttagttcttttatgtcaagaaggcactggtttagttcatctataaatgtcacgccataactaccacgcatgctatactcagaattatgataaagatatttatccttggggtgtaggtaatggggattaatctttgttggatcagtctctaagttaacacatgtcacacatgatccatttatagttatatcacacgatgatgatttatgaattgattccggaagatcgctatcgtattttgtggttccacaattcatttccatacatgttattgtcacactaatattatgatgaactttatctagccgctgagtggtaaacaacagaacagatagtttattatctttaccaacaccctcagccgctgccacaaatctctgatccgtatccatgatggtcatgtttatttctagtccgtatccagtcaacactatgttagcatttctgtcgatatagctttcactcatatgacactcaccaataatagtagaattaatgtcgtaatttacaccaatagtgagttcggcggcaaagtaccaataccggtaatcttgtcgaggaggacatatagtattcttgtattctaccgaatacccgagagatgcgatacaaaagagtaagactaatttgtaaaccatcttactcaaaatatgtaacaatagtacgatgcaatgagtaagacaataggaaatctatcttatatacacataattattctatcaattttaccaattagttagtgtaatgttaacaaaaatgtgggagaatctaattagtttttctttacacaattgacgtacatgagtctgagttccttgtttttgctaattatttcatccaatttattattcttgactatatcgagatcttttgtataggagtcagacttgtattcaacatgcttttctataatcattttagctatttcggcatcatccaatagtacattttccagattagcagaatagatattaatgtcgtatttgaacagagcctgtaacatctcaatgtctttattatctatagccaatttaatgtccggaatgaagagaagggaattattggtgtttgtcgacgtcatatagtcgagcaagagaatcatcatatccacgtgtccattttttatagtgatgtgaatacaactaaggagaatagccagatcaaaagtagatggtatctctgaaagaaagtaggaaacaatacttacatcattaagcatgacggcatgataaaatgaagttttccatccagttttcccatagaacatcagtctccaatttttcttaacaaacagttttaccgtttgcatgttaccactatcaaccgcataatacaatgcggtgtttcccttgtcatcaaattgtgaatcatccagtccactgaatagcaaaatctttactattttggtatcttccaatgtggctgcctgatgtaatggaaattcattctctagaagatttttcaatgctccagcgttcaacaacgtacatactagacgcacgttattatcagctattgcataatacaaggcactatgtccatggacatccgccttaaatgcatctttgctagagagaaagcttttcagctgcttagacttccaagtattaattcgtgacagatccatgtctgaaacgagacgctaattagtgtatattttttcattttttataattttgtcatattgcaccagaattaataatatctctaatagatctgattagtagatacatggctatcgcaaaacaacatatacacatttaataaaaataatatttattaagaaaattcagatttcacgtacccatcaatataaataaaataatgattccttacaccgtacccatattaaggagattccaccttacccataaacaatataaatccagtaatatcatgtctgatgatgaacacaaatggtgtattaaattccagtttttcaggagatgatctcgccgtagctaccataatagtagatgcctctgctacagttccttgttcgtcgacatctatctttgcattctgaaacattttataaatatataatgggtccctagtcatatgtttaaacgacgcattatctggattaaacatactaggagccatcatttcggctatcgacttaatatccctcttattttcgatagaaaatttagggagtttaagattgtacactttattccctaattgaaacgaccaatagtctaattttgcagccgtaatagaatctgtgaaatgggtcatattatcacctattgccaggtacatactaatattagcatccttatacggaaggcgtaccatatcatattcttcgtcatcgattgtgattgtatttccttgcaatttagtaactacgttcatcatgggaaccgttttcgtaccgtacttattagtaaaactagcattgcgtgttttagtgatatcaaacggatattgccatatacctttaaaatatatagtattaatgattgcccatagagtattattgtcgagcatattagaatctactacattagacataccggatctacgttctactatagaattaattttattaaccgcatctcgtctaaagtttaatctatataggccgaatctatgatattgttgataatacgacggtttaatgcacacagtattatctacgaaactttgataagttagatcagtgtacgtatatttagatgttttcagcttagctaatcctgatattaattctgtaaatgctggacccagatctctttttctcaaatccatagtcttcaataattctattctagtattacctgatgcaggcaatagcgacataaacatagaaaacgaataaccaaacggtgagaagacaatattatcatcttgaatatttttatacgctactataccggcattggtaaatccttgcagacgataggtagacactgaacacgttaacgatagtatcaataacgcaatcatgattttatggtattaataattaaccttatttttatgttcggtataaaaattattgatgtctacacatccttttgtaattgacatctatatatccttttgtataatcaactctaatcactttaacttttacagttttccctaccagtttatccctatattcaacatatctatccatatgcatcttaacactctctgccaagatagcttcaaagtgaggatagtcaaaaagataaatatatagagcataatccttctcgtatactctgccctttattacatcacccgcattgggcaacgaataacaaaatgcaagcatcttgttaacgggctcgtaaattgggataaaaattatgtttttatatctattttattcaagagaatattcaggaatttctttttccggttgtatctcatcgcagtatatatcatttgtacattgtttcatattttttaatagtctacaccttttagtaggactagtatcgtacaattcatagctgtattttgaattccaatcacgcataaaaatatcttccaattgttgacgaagacctaatccatcatccggtgtaatattaatagatgctccacatgtatccgtaaagtaatttcctgtccaatttgaggtacctatatacgccgttttatcggttaccatatatttggcatggtttaccctagaatacggaatgggaggatcagcatctggtacaataaatagctttacttctatatttatgtttttagattttagcatagcgatagatcttaaaaagtttctcatgataaacgaagatcgttgccagcaactaatcaatagcttaactgacacttgtctgtctatagcggctcttcttaattcatcttctatataaggccaaaacaaaatattgcctgccttcgaataaataatagggataaagttcataacagatacataaacgaatttactcgcatttctgatacatgacaataaagcggttaaatcattggttctttccatagtacatagttgttgcggtgcagaagcaataaatacagagtgtggaacgccgcttacgttaatactaagaggatgatctgtattataatacgacggataaaagtttttccaattatatggtagattgttaactccaagataccagtatacctcaaaaatttgagtgagatccgctgccaagttcctattattgaagatcgcaatacccaattctttgacctgagttagtgatctccaatccatgttagcgcttcctaaataaatatgtgtattatcagatatccaaaattttgtatgaagaactcctcctaggatatttgtaatatctatgtatcgtacttcaactccggccatttgtagtctttcaacatcctttaatggtttgttagatttattgacggctactctaactcgtactcctcttttgggtaattgtacaatcttgtttaatattatcgtgccgaaattcgtacccacttcatccgataaactccaataaaaagatgatatatctagtgtttttgtggtattggatagaatttccctccacatgttaaatgtagacaaatatactttatcaaattgcatacctataggaatagtctctgtaatcactgcgattgtattatccggattcattttatttgttaaaagaataatcctatatcacttcactctattaaaaatccaagtttctatttctttcatgactgattttttaacttcatccgtttccttatgaagatgatgtttggcaccttcataaatttttatttctctattacaatttgcatgttgcatgaaataatatgcacctaaaacatcgctaatcttattgtttgttccctggagtatgagagtcggggggtgttaatcttggaaattatttttctaaccttgttggtagccttcaagacctgactagcaaatccagccttaattttttcatgattgattaatgggtcgtattggtatttataaactttatccatatctctagatactgattctggacatagctttccgactggcgcatttagtgtgatggttcccataagtttggcagctagcagattcagttttgaaacagcatctgcattaactagaggagacattagaatcattgctgtaaacaagtttggattatcgtaagaggctagctcccatggaatgacccaataagtagatttaatagttaccacgtgctgtaccaaagtcatcaatcatcattttttcaccattacttcttccatgtccaatatgatcatgtgagaatactaaaattcctaacgatgatatgttttcagctagttcgtcataacgtccagaatgtttaccagctccatgacttatgaatactaatgccttaggatatgtaataggtttccaatatatgtaatcattgtccagattgaacatacagtttgcactcatgattcacgttatataactatcaatattaacagttcgtttgatgatcatattatttttatgttttattgataattgtaaaaacatacaattaaatcaatatagaggaaggagacggctactgtcttttgtgagatagtcatggcgactaaattagattatgaggatgctgttttttactttgtggatgatgataaaatatgtagtcgcgactccatcatcgatctaatagatgaatatattacgtggagaaatcatgttatagtgtttaacaaagatattaccagttgtggaagactgtacaaggaattgatgaagttcgatgatgtcgctatacggtactatggtattgataaaattaatgagattgtcgaagctatgagcgaaggagaccactacatcaattttacaaaagtccatgatcaggaaagtttattcgctaccataggaatatgtgctaaaatcactgaacattggggatacaaaaagatttcagaatctagattccaatcattgggaaacattacagatctgatgaccgacgataatataaacatcttgatactttttctagaaaaaaaattgaattgatgatataggggtcttcataacgcataattattacgttagcattctatatccgtgttaaaaaaaattatcctatcatgtatttgagagttttatatgtagcaaacatgatagctgtgatgccaataagctttagatattcacgcgtgctagtgttagggatggtattatctggtggtgaaatgtccgttatataatctacaaaataatcatcgcatatagtatgcgatagtagagtaaacatttttatcgtttctactgggttcatacatcgtctacccaattcggttataaatgaaattgtcgccaatcttacacccaaccccttgttatccattagtatagtattaacttcgttatttatgtcataaactgtaaatgattttgtagatgccatatcatacatgatattcatgtccctattataatcattactaactttatcacaatatatgttgataatatctatatatgatctagtctttgtgggcaactgtctatacaagtcgtctaaacgttgtttactcatatagtatcgaacagccatcattacatggtcccgttccgttgatagataatcgagtatgttagtggacttgtcaaatctatataccatattttctggaagtggatatacatagtcgtgatcaacattattgctagcctcatcttctatatcctgtactataccattatctatatcatctacataatctacgatattattacacataaacatcgacaacatactattgtttattatctaagtcctgttgatccaaacccttgatctcctctatttgtactatctagagattgtacttcttccagttctggataatatatacgttgatagattagctgagctattctatctccagtatttacattaaacgtacattttccattattaataagaatgactcctatgtttcccctataatcttcgtctattacaccgcctcctatatcaatgccttttagggacagaccagacctaggagctattctaccatagcagaacttaggcatggacatactaatatctgtcttaattaactgtcgttctcctggagggatagtataatcgtaagcgctatataaatcatatccggcggcgtaaggtgattgcctagtaggagatttagctctgttagtttccttaacaaatctaactggtgagttaatattcatgttgaacataaaactaatattttatttcaaaattatttaccatcccatatattccatgaataagtgtgatgattgtacacttctatagtatctatatacgattcacgataaaatcctcctatcaatagcagtttattatccactatgatcaattctggattatccctcggataaataggatcatctatcagagtccatgtattgctggattcacaataaaattccgcatttctaccaaccaagaataaccttctaccgaacactaacgcgcatgatttataatgaggataataagtggatggtccaaactgccactgatcatgattgggtagcaaatattctgtagttgtatcagtttcagaatgtcctcccattacgtatataacattgtttatggatgccactgctggattacatctaggtttcagaagactcggcatattaacccaagcagcatccccgtggaaccaacgctcaacagatgtgggatttggtagacctcctactacgtataatttattgttagcgggtatcccgctagcatacagtctggggctattcatcggaggaattggaatccaattgtttgatatataatttaccgctatagcattgttatgtatttcattgttcatccatccaccgatgagatatactacttctccaacatgagtacttgtacacatatggaatatatctataatttgatccatgttcataggatactctatgaatggatacttgtatgatttgcgtggttgtttatcacaatgaaatattttggtacagtctagtatccattttacattatttatacctctgggagaaagataatttgacctgattacatttttgataaggagtagcagatttcctaatttatttcttcgctttatataccacttaatgacaaaatcaactacataatcctcatctggaacatttagttcatcgctttctagaataagtttcatagatagataatcaaaattgtctatgatgtcatcttccagttccaaaaagtgtttggcaataaagtttttagtatgacataagagattggatagtccgtattctatacccatcatgtaacactcgacacaatattcctttctaaaatctcgtaagataaagtttatacaagtgtagatgataaattctacagaggttaatatagaagcacgtaataaattgacgacgttatgactatctatatatacctttccagtatacgagtaaataactatagaagttaaactgtgaatgtcaaggtctagacaaacccttgtaactggatctttatttttcgtgtatttttgacgtaaatgtgtgcgaaagtaaggagataactttttcaatatcgtagaattgactattatattgccacctatagcatcaataattgttttgaatttcttagtcatagacaatgctaatatattcttacagtacacagtattaacaaatatcggcatttatgtttctttaaaagtcaacatctaaagaaaaatgattatcttcttgagacataactcccattttttggtattcacccacacgtttttcgaaaaaattagtttttccttccaatgatatattttccatgaaatcaaacggattggtaacattataaatttttttaaatcccaattcagaaatcaatctatccgcgacgaattctatatatgttttcatcatttcacaattcattcctataagtttaactggaagagccgcagtaagaaattcttgttcaatggataccgcatctgttataatagatctaacggtttcttcactcggtggatgcaataaatgtttaaacatcaaacatgcgaaatcgcagtgcagaccctcgtctctactaattagttcgttggaaaacgtgagtccgggcattaggccacgctttttaagccaaaatatggaagcgaatgatccagaaaagaagattccttctactgcagcaaaggcaataagtctctctccataaccggcgctgtcatgtatccacttttgagcccaatcggccttcttttttacacaaggcatcgtttctatggcattaaagagatagtttttttcattactatctttaacataagtatcgatcaaaagactatacatttccgaatgaatgttttcaatggccatctgaaatccgtagaaacatctagcctcggtaatctgtacttctgtacaaaatcgttccgccaaattttcattcactattccgtcactggctgcaaaaaacgccaatacatgttttataaaatatttttcgtctggtgttagtttattccaatcattgatatctttagatatatctacttcttccactgtccaaaatgatgcctctgcctttttatacatgttccagatgtcataatattggattgggaaaataacaaatctatttggatttggtgcaaggatgggttccataactaaattaacaataacaataaattttttttcagttatctatatgcctgtacttggatcttttgtacatcgatatcgccgcaatcactacaataattacaagtattattgatagcattgttattagtactatcataattaaattatcgacattcatgggtgctgaataatcgttattatcatcattatcattttgtaattgtgacatcatactagataaatcgtttgcgagattgttgtgggaagcgggcatggaggatgaattatcgttattattatttaacgcctcccattcggattcacaaatgttacgcacattcaacattttatggaaactataattttgtgaaaacagataacaagaaaactcgtcatcgttcaaatttttaacgatagtaaaccgattaaacgtcgagctaatttctaacgctagcgactctgttggatatgggtttccagatatatatcttttcagttcccctacgtatctataatcatctgtaggaaatggaagatatttccatttatctactgttcctaatatcatatgtggtggtgtagtagaaccattaagcgcgaaagatgttatttcgcatcgtattttaacttcgcaataatttctggttagataacgcactctaccagtcaagtcaatgatattagcctttacagatatattcatagtagttgtaacgatgactccatcttttagatgcgatactcctttgtatgtaccagaatcttcgtaccgcaaactcgatatatttaaacaagttaatgagatattaacgcgttttatgaatgatgatatataaccagaagttttatcctcggtggctagcgctataaccttatcattataataccaactagtgtgattaatatgtgacacgttagtgtgggtacaaatatgtacattatcgtctacgtcgtattcgatacatccgcatacagccaacaaatataaaatgacaaatactctaacgccgttcgtacccatcttgatgcggtttaataaatgttttgatttcaatttattgtaaaaaaagattcggttttatactgttcgatattctcattgcttatattttcatctatcatctccacacagtcaaatccgtggttagcatgcacctcatcaaccggtaaaagactatcggactcttctatcattataactctagaatatttaatttggtcattattaatcaagtcaattatcttatttttaacaaacgtgagtattttactcattttttataaaaacttttagaaatatacagactctatcgtgtgtctatatcttctttttatatccaatgtatttatgtctgatttttcttcatttatcatatataatggtccaaattctacacgtgcttcggattcatccagatcattaaggttcttataattgtaacatccttctcttccctcttctacatcttccttcttattcttattcttagcgtcacagaatctaccacagcaggatcccatgacgagcgtcatattaaactaatccattttcaattataatatacgattagtaatgaccattaaaataaaaaatattcttcataaccggcaagaaagtgaaaagttcacattgaaactatgtcagtagtatacatcatgaaatgatgatatatatatactctattttggtggaggattatatgatataattcgtggataatcattcttaagacacatttcttcattcgtaaatcttttcacgttaaatgagtgtccatattttgcaatttcttcatatgatggcggtgtacgtggacgaagctgctcctgttcttgttgtagtcgccgactgtcgtgtttgcgtttagatccctccattatcgcgattgcgtagatggagtactattatataccttgtaattaaatttttttattaattaaacgtataaaaacgttccgtatctgtatttaagagccagatttcgtctaatagaacaaatagctacagtaaaaataactagaataattgctacacccactagaaaccacggatcgtaatacggcaatcggttttcgataataggtggaacgtatattttatttaaggacttaacaattgtctgtaaaccacaatttgcttccgcggatcctgtattaactatctgtaaaagcatatgttgaccgggcggagccgaacattctccgatatctaatttctgtatatctataatattattaacctccgcatacgcattacagttcttttctagcttggataccgcactaggtacatcgtctagatctattcctatttcttcagcgatagctcttctatccttttccggaagcaatgaaatcacttcaataaatgattcaaccatgagtgtgaaactaagtcgagaattactcatgcatttgttagttattcggagcgcgcaatttttaaactgtcctataacctctcctatatgaatagcacaagtgacattagtagggatagaatgttgagctaatttttgtaaataactatctataaaaagattatacaaagttttaaactctttagtttccgccatttatccagtctgagaaaatgtctctcataataaatttttccaagaaactaattgggtgaagaatggaaacctttaatctatatttatcacagtctgttttggtacacatgatgaattcttctaatgctgtactaaattcgatatctttttcgatttctggatatgtttttaataaagtatgaacaaagaaatggaaatcgtaataccagttatgttcaactttgaaattgttttttattttcttgttaatgattccagccacttgggaaaagtcaaagtcgtttaatgccgatttaatacgttcattaaaaacaaactttttatcctttagatgaattattattggttcattggaatcaaaaagtaagatattatcgggtttaagatctgcgtgtaaaaagttgtcgcaacagggtagttcgtagattttaatgtataacagagccatctgtaaaaagataaactttatgtattgtaccaaagatttaaatcctaatttgatagctaactcggtatctactttatctgccgaatacagtgctaggggaaaaattataatatttcctctttcgtattcgtagttagttctcttttcatgttcgaaaaagtgaaacatgcggttaaaatagtttataacattaatattactgttaataactgccggataaaagtgggatagtaatttcacgaatttgatactgtcctttctctcgttaaacgcctttaaaaaaactttagaagaatatctcaatgagagttcctgaccatccatagtttgtatcaataatagcaacatatgaagaacccgtttatacagagtatgtaaaaatgttaatttatagtttaatcccatggcccacgcacacacgattaattttttttcatctccctttagattgttgtatagaaatttgggtactgtgaactccgccgtagtttccatgggactatataattttgtggcctcgaatacaaattttactacatagttatctatcttaaagactataccatatcctcctgtagatatgtgataaaaatcgtcgtttataggataaaatcgtttatccttttgttggaaaaaggatgaattaatgtaatcattctcttctatctttagtagtgtttccttattaaaattcttaaaataatttaacaatctaactgacggagcccaattttggtgtaaatctaattgggacattatgttgttaaaatacaaacagtctcctaatataacagtatctgataatctatggggagacatccattgatattcaggggatgaatcattggcaacacccatttattgtacaaaaagccccaatttacaaacgaaagtccaggtttgatagagacaaacaattaactattttgtctctgtttttaatttctttggtaatgaaattattcacaatatcagtatcttctttatctaccagagattttactaacttgataaccttggctgtctcattcaatagggtagtaatatttgtatgtgtgatattgatatctttttgaattgtttcttttagaagtgattctttgatggtgccagcatacgaattacaataatgcagaaactcagttaacatgcaggaattatagtaagccaattccaattgttgcctgtattgtattagagtattaatatgcgcaatggtgtccttgcgtttctctgatagaatgcgagcagcgattttggcgttatcatttgacgatatttctggaatgacgaatcctgtttctactaactttttggtaggacaaagtgaaacaatcaagaagatagcttctcctcctatttgtggaagaaattgaactcctctagatgatctactgacgatagtatctccttgacagatattggaccgaattacagaagtacctggaatgtaaagccctgaaaccccctcattttttaagcagattgttgccgtaaatcctgcactgtgaccaagatagagagctcctttggtgaatccatctctatgtttcagtttaaccaagaaacagtcagctggtctaaaatttccatctctatctaatacagcatctaacttgatgtcaggaactatgaccggtttaatgttatatgtaacattgagtaaatccttaagttcataatcatcactgtcatcagttatgtacgatccaaacaatgtttctactggcatagtggatacgaagatgctatccatcagaatgtttccctgattagtattttctatatagctattcttctttaaacgattttccaaatcagtaactatgttcatttttttaggagtaggacgcctagccagtatggaagaggattttctagatcctctcttcaacatctttgatctcgatggaatgcaaaaccccatagtgaaacaaccaacgataaaaataatattgtttttcactttttataattttaccatctgactcatggattcattaatatctttataagagctactaacgtataattctttataactgaactgagatatatacaccggatctatggtttccataattgagtaaatgaatgctcggcaataactaatggcaaatgtatagaacaacgaaattatactagagttgttaaagttaatattttctatgagctgttccaataaattatttgttgtaactgcgttcaagtcataaatcatcttgatactatccagtaaaccgtgtttaagttctggaatattatcatcccattgtaaagcccctaattcgactatcgaatatcctgctctgatagcagtttcaatatcgacggacgtcaatactgtaataaaggtggtagtattgtcatcatcgtgataaactacgggaatatggtcgttagtaggtacggtgactttacacaacgcgatatataactttccttttgtaccatttttaacgtagttgggacgtcctgcagggtattgttttgaagaaatgatatcgagaacagatttgatacgatatttgttggattcctgattattcactataatataatctagacagatagatgattcgataaatagagaaggtatatcgttggtaggataatacatccccattccagtattctcggatactctattgatgacactagttaagaacatgtcttctattctagaaaacgaaaacatcctacatggactcattaaaacttctaacgctcctgattgtgtctcgaatgcctcgtacaaggatttcaaggatgccatagattctttgaccaacgatttagaattgcgtttagcatctgatttttttattaaatcgaatggtcggctctctggtttgctaccccaatgataacaatagtcttgtaaagataaaccgcaagaaaatttatacgcatccatccaaataaccctagcaccatcggatgatattaatgtattattatagattttccatccacaattattgggccagtatactgttagcaacggtatatcgaatagattactcatgtaacctactagaatgatagttcgtgtactagtcataatatctttaatccaatctaagaaatttaaaattagattttttacactgttaaagttaacaaaggtattacccggatacgtggatatcatatatggcattggtccattatcagtaatagctccataaactgatacggcgatggtttttatatgtgtttgatctaacgaggaagaaattcgcgcccacaattcatctctagatatgtatttaatatcaaacggtaacacatcaatttcgggacgcgtatatgtttctaaatttttaatccaaatataatgatgacctatatgccctattatcatactgtcaactatagtacacctagagaacttacgatacatctgtttcctataatcgttaaattttacaaatctataacatgctaaaccttttgacgacagccattcattaatttctgatatggaatctgtattctcgataccgtattgttctaaagccagtgctatatctccctgttcgtgggaacgctttcgtataatatcgatcaacggataatctgaagtttttggagaataatatgactcatgatctatttcgtccataaacaatctagacataggaattggaggcgatgatcttaattttgtgcaatgagtcgtcaatcctataacttctaatcttgtaatattcatcatcgacataatactatctatgttatcatcgtatattagtataccacggccttcttcatttcgtgccaaaataatatacagtcttaaataattacgcaatatctcaatagtttcataattgttagctgttttcatcaagatttgtaccctgtttaacatgatggcgttctatacgtctctattttcttttttttaaatttttaacgatttactgtggctagatacccaatctctctcaaatatttttttagcctcgcttacaagctgtttatctatactattaaaactgacgaatccgtgattttggtaatgggttccgtcgaaatttgccgaagtgatatgaacatattcgtcgtcgactatcaacaattttgtattattctgaatagtgaaaaccttcacagatagatcattttgaacacacaacgcgtctagacttctggcggttgccatagaatatacgtcgttcttatcccaattaccaactagaagtctgatcttaactcctctattaatggctgcttctataatggagttgtaaatgtcgggccaatagtagctattaccgtcgacacgtgtagtgggaactatggccaaatgttcaatatctatactagtcttagccgacttgagtttatcaataactacatcggtatctagatctctagaatatcccaataggtgttccggagaatcagtaaagaacactccacctataggattcttaatatgatacgcagtgctaactggcaaacaacaagccgcagagcataaattcaaccatgaattttttgcgctattaaaggctttaaaagtatcaaatcttctacgaagatctgtggccagcgggggataatcagaatatacacctaacgttttaatcgtatgtatagatcctccagtaaatgacgcgtttcctacataacatctttcatcatctgacacccaaaaacaaccgagtagtagtcccacattattttttttatctatattaacggttataaaatttatatccgggcagtgactttgtagctctcccagatttcttttccctcgttcatctagcaaaactattattttaatccctttttcagatgcctcttttagtttatcaaaaataagcgctcccctagtcgtactcagaggattacaacaaaaagatgctatgtatatatatttcttagctagagtgataatttcgttaaaacattcaaatgttgttaaatgatcggatctaaaatccatattttctggtagtgtttctaccagcctacattttgctcccgcaggtaccgatgcaaatggccacatttagttaacataaaaacttatacatcctgttctatcaacgattctagaatatcatcggctatatcgctaaaattttcatcaaagtcgacatcacaacctaactcagtcaatatattaagaagttccatgatgtcatcttcgtctatttctatatccgtatccattgtagattgttgaccgattatcgagtttaaatcattactaatactcaatccttcagaatacaatctgtgtttcattgtaaatttataggcggtgtatttaagttggtagattttcaattatgtatcaatatagcaacagtagttcttgctcctccttgattctagcatcctcttcattattttcttctacgtacataagcatgtccaatacgttagacaacacaccgacgatggcggccgccacagacacgaatatgactagaccgatgaccatttaaaaacccctctctagctttcacttaaactgtatcgattattcttttagaacatgtataatataaaaacattattctatttcgaatttaggcttccaaaaatttttcatccgtaaaccgataataatatatatagacttgttaatagtcggaataaatagattaatgcttaaactatcatcatctccacgattagagatacaatatttacattttttttgctgtttcgaaactttatcaatacacgttaatacaaacccaggaaggagatattgaaactgaggctgttgaaaatgaaacggtgaatacaataattcagataatgtaaaatcatgattccgtattctgatgatattagaactgctaatggatgtcgatggtatgtatctaggagtatctattttaacaaagcatcgatttgctaatatacaattatccttttgattaattgttattttattcatattcttaaaaggtttcatatttatcaattcttctacattaaaaatttccatttttaatttatgtagcccccgcaatactcctcattacgtttcattttttgtctataatatccattttgttcatctcggtacatagattatccaattgagaagcgcatttagtagttttgtacattttaagtttattgacgaatcgtcgaaaactagttatagttaacattttattatttgataccctgatattaatacccctgccgttactattatttataactgatgtaatccacgtaacattagaattaattatcgatagtaatgcatcgacgcttccaaaattgtctattataaactcaccgataatttttttattgcatgttttcatattcattaggattatcaaatctttaatcttactacgattgtatgcgttgatattgcaagacgtcattctaaaagacggaggatctccatcaaatgccagacaatcacgtacaaagtacatggaaataggttttgttctattgcgcatcatagatttatatagaacacccgtagaaatactaatttgttttactctataaaatactaatgcatctatttcatcgttttgtataacgtctttccaagtgtcaaattccaaatttttttcattgatagtaccaaattcttctatctctttaactacttgcatagataggtaattacagtgatgcctacatgccgttttttgaaactgaatagatgcgtctagaagcgatgctacgctagtcacaatcaccactttcatatttagaatatatgtatgtaaaaatatagtagaatttcattttgtttttttctatgctataaatgaattctcattttgcatctgctcatactccgttttatatcaataccaaagaaggaagatatctggttctaaaagccgttaaagtatgcgatgttagaactgtagaatgcgaaggaagtaaagcttcctgcgtactcaaagtagataaaccctcatcgcccgcgtgtgagagaagaccttcgtccccgtccagatgcgagagaatgaataaccctggaaaacaagttccgtttatgaggacggacatgctacaaaatatgttcgcggctaatcgcgacaacgtggcgtcgagacttttgaactaaaatacaattatatccttttcgatattaataaatccgtgtcgtccaggttttttatctctttcagtatgtgaatagataggtattttatctctattcatcatcgaatttaagagatccgataaacattgtttgtattctccagatgtcagcatctgatacaacaatatatgtgcacataaacctctggcacttatttcatgtaccttccccttatcactaaggagaatagtatttgagaaatatgtatacatgatattatcatgaattagatatacagaatttgtaacactctcgaaatcacacgatgtgtcggcgttaagatctaatatatcactcgataacacattttcatctagatacactagacattttttaaagctaaaatagtctttagtagtgacagtaactatgcgattattttcatcgatgatacatttcatcggcatattattacgcttaccatcaaagactataccatgtgtatatctaacgtattctagcatggttgccatacgcgcattaaacttttcaggatctttggatagatcttccaatctatctatttgagaaaacatttttatcatgttcaatagttgaaacgtcggatccactatatagatattatctataaagattttaggaactacgttcatggtatcctggcgaatattaaaactatcaatgatatgattatcgttttcatcttttatcaccatatagtttctaagatatgggattttacttaatataatattatttcccgtgataaattttattagaaaggccaaatctataagaaaagttctagaattagtctgaagaatatctatatcgccgtaccgtatatttggattaattagatatagagaatatgatccgtaacatatacaacttttattatggcgtctaagatattcttccatcaacttattaacatttttgactagggaagatacattatgacgtcccattacttttgccttgtctattactgcgacgttcatagaatttagcatatctcttgccaattcttccattgatgttacattataagaaattttagatgaaattacatttggagctttaatagtaagaactcctaatatgtccgtgtatgtggtcactaatacagattgtagttctataatcgtaaataatttacctatattatatgtttgagtctgtttagaaaagtagctaagtatacgatcttttatttctgatgcagatgtattaacatcggaaaaaaatctttttttattcttttttactaaagatacaaatatgtctttgttaaaaacagttattttttgaatatttctagcttgtaattttaacatatgatattcgttcacactaggtactctgcctaaataggtttctataatctttaatgtaatattaggaagagtattctgatcaggattcctattcattttgaggatttaaaactctgattattgtctaatatggtctctacgcaaactttttcacagagcgatagagtttttgataactcgtttttcttaagaaatataaaactactgtctccagagctcgctctatcttttattttatttaattcgatacaaactcctgatactggttcagaaagtaattcattaattttcagtcctttatagaagatatttaatatagataatacaaaattttcagttcttgatatcgatctgattgatcctagaactagatatattaataacgtgctcattaggcagtttatggcagcttgataattagatatagtatattccagttcatatttattagataccgcattgcccagattttgatattctatgaattcctctgaaaataaatccaaaataactaaacattctattttttgtggattagtgtactctcttccctctatcatgttcactactggtgtccacgatgataaatatctagagggaatataatatagtccataggatgccaatctagcaatgtcgaataactgtaatttgattcttcgttcttcattatgaattgattcttgaggtataaacctaacacaaattatattattagacttttcgtatgtaatgtctttcatgttataagtttttaatcctggaatagaatctattttaatgaggcttttaaacgcagagttctccaacgagtcaaagcataatactctgttgtttttcttatatacgatgttacgattttcttctttgaatggaataggtttttgaattagtttataattacaacataatagataaggaagtgtgcaaatagtacgcggaaaaaacataatagctcccctgttttcatccatggttttaagtaaatgatcactggcttctttagtcaatggatattcgaacattaaccgtttcatcatcattggacagaatccatatttcttaatgtaaagagtgatcaaatcattgtgtttattgtaccatcttgttgtaaatgtgtattcggttatcggatctgctcctttttctattaaagtatcgatgtcgatctcgtctaagaattcaactatatcgacatatttcatttgtatacacataaccattactaacgtagaatgtataggaagagatgtaacgggaacagggtttgttgattcgcaaactattctaatacataattcttctgttaatacgtcttgcacgtaatctattatagatgccaagatatctatataattattttgtaagatgatgttaactatgtgatctatataagtagtgtaataattcatgtatttcgatatatgttccaactctgtctttgtgatgtctagtttcgtaatatctatagcatcctcaaaaaatatattcgcatatattcccaagtcttcagttctatcttctaaaaaatcttcaacgtatggaatataataatctattttacctcttctgatatcattaatgatatagtttttgacactatcttctgtcaattgattcttattcactatatctaagaaacggatagcgtccctaggacgaactactgccattaatatctctattatagcttctggacataattcatctattataccagaattaatgggaactattccgtatctatctaacatagttttaagaaagtcagaatctaagacctgatgttcatatattggttcatacatgaaatgatctctattgatgatagtgactatttcattctctgaaaattggtaactcattctatatatgctttccttgttgatgaaggatagaatatactcaatagaatttgtaccaacaaactgttctcttatgaatcgtatatcatcatctgaaataatcatgtaaggcatacatttaacaattagagacttgtctcctgttatcaatatactattcttgtgataatttatgtgtgaggcaaatttgtccacgttctttaattttgttatagtagatatcaaatccaatggagctacagttcttggcttaaacagatatagtttttctggaacgaattctacaacattattataaaggactttgggtagataagtgggatgaaatcctattttaattaatgcgatagccttgtcctcgtgcagatatccaaacgcttttgtgatagtatggcattcattgtctagaaacgctctacgaatatctgtgacagatatcatctttagagaatatactagtcgcgttaatagtactacaatttgtattttttaatctatctcaataaaaaaattaatatgtatgattcaatgtataactaaactactaactgttattgataactagaatcagaatctaatgatgacgtaaccaagaagtttatctactgccaatttagctgcattatttttagcatctcgtttagattttccatctgccttatcgaatactcttccgtcgatgtctacacaggcataaaatgtaggagagttactaggccccactgattcaatacgaaaagaccaatctctcctagtaatttggcagtactcattaataacggtgacagggttagcacctttccaatcaataatttttttagccggaataacatcatcaaaagacttatgatcctctctcattgatttttcgcgggatacatcatctattatgacgtcagccatagcatcagcatccggcttatccgcctccgttgtcataaaccaacgaggaggaatatcgtcggagctgtacaccatagcactacgttgaagatcgtacagagctttattaacttctcgcttctccatattaagttgtctagttagttgtgcagcagtagctccttcgattccaatggttttaatagcctcacacacaatctctgcgttagaacgctcgtcgatatagattttagacatttttagagagaactaacgcaatcagtaataaaactaatttattttatcattttttttattcatcatcctctggtggttcgtcgtttctatcgaatgtagctctgattaacccgtcatctataggtgatgctggttctggagattctggaggagatggattattatctggaagaatctctgttatttccttgttttcatgtatcgattgcgttgtaacattaagattgcgaaatgctctaaatttgggaggcttaaagtgttgtttgcaatctctacacgcgtgtctaactagtggaggttcgtcagcggctctagtttgaatcatcatcggcgtagtattcctacttttacagttaggacacggtgtattgtatttctcgtcgagaacgttaaaataatcgttgtaactcacatcctttattttatctatattgtattctactcctttcttaatgcattttataccgaataagagatagcgaaggaattctttttcggtgccgctagtacccttaatcatatcacatagtgttttatattccaaatttgtggcaatagacggtttatttctatacgatagtttgtttctggaatcctttgagtattctataccaatattattctttgattcgaatttagtttcttcgatattagattttgtattacctatattcttgatgtagtactttgatgatttttccatggcccattctattaagtcttccaagttggcatcatccacatattgtgatagtaattctcggatatcagtagcggttaccgccattgatgtttgttcattggatgagtaactactaatgtatacattttccatttataacacttatgtattaactttgttcatttatattttttcattattatgttgatattaacaaaagtgaatatatatatgttaataattgtattgtggttatacggctacaattttataattagtgaaagtcagtgtccgatgatcaatgacgatagctttactctgaaaagaaagtatcaaatcgatagtgcggagtcaacaataaaaatggataagaagaggacaaagtttcagaatagagccaaaatggtaaaagaaataaatcagacaataagagcagcacaaactcattacgagacattgaaactaggatacataaaatttaagagaatgattaggactactactctagaagatatagcaccatctattccaaataatcagaaaacttataaactattctcggacatttcagccatcggcaaagcatcacggaatccaagtaagatggtatatgctctgctgctttacatgtttcccaatttgtttggagatgatcatagattcattcgttatagaatgcatccaatgagtaaaatcaaacacaagatcttctctcctttcaaacttaatcttattagaatattagtggaagaaagattctataataatgaatgcagatctaataaatggagaataattggaacacaagttgataaaatgttgatagctgaatctgataaatatacaatagatgcaaggtataacctaaaacccatgtatagaatcaagggaaaatctgaagaagataccctctttatcaaacagatggtagaacaatgtgtgacatcccaggaattggtggaaaaagtgttgaagatactgtttagagatttgttcaagagtggagaatacaaagcgtacagatacgatgatgatgtagaaaatggatttattggattggatacactaaaattaaacattgttcatgatatagttgaaccatgtatgcctgttcgtaggccagtggctaagatactgtgtaaagaaatggtaaataaatactttgagaatccgctacatattattggtaaaaatcttcaagagtgcattgactttgttagtgaataggcatttcatctttctccaatactaattcaaattgttaaattaataatggatagtataaatagttattagtgataaaatagtaaaaataattattagaataagagtgtagtatcatagataactctcttctataaaaatggattttattcgtagaaagtatcttatatacacagtagaaaataatatagattttttaaaggatgatacattaagtaaagtaaacaattttaccctcaatcatgtactagctctcaagtatctagttagcaattttcctcaacatgttattactaaggatgtattagctaataccaatttttttgttttcatacatatggtacgatgttgtaaagtgtacgaagcggttttacgacacgcatttgatgcacccacgttgtacgttaaagcattgactaagaattatttatcgtttagtaacacaatacaatcgtacaaggaaaccgtgcataaactaacacaagatgaaaaatttttagaggttgccaaatacatggacgaattaggagaacttataggcgtaaattatgacttagttcttaatccattatttcacggaggggaacccatcaaagatatggaaatcatttttttaaaactgtttaagaaaacagacttcaaagttgttaaaaaattaagtgttataagattacttatttgggcttacctaagcaagaaagatacaggcatagagtttgcggataatgatagacaagatatatatactctatttcaacaaactggtagaatcgtccatagcaatctaacagaaacgtttagagattatatctttcccggagataagactagctattgggtgtggttaaacgaaagtatagctaatgatgcggatattgttcttaatagacacgccattaccatgtatgataaaattcttagttatatatactctgagataaaacaaggacgcgttaataaaaacatgcttaagttagtttatatctttgagcctgaaaaagatatcagagaacttctgctagaaatcatatatgatattcctggagatatcctatctattattgatgcaaaaaacgacgattggaaaaaatattttattagtttttataaagctaattttattaacggtaatacatttattagtgatagaacgtttaacgaggacttattcagagttgttgttcaaatagatcccgaatatttcgataatgaacgaattatgtctttattctctacgagtgctgcggacattaaacgatttgatgagttagatattaataacagttatatatctaatataatttatgaggtgaacgatatcacattagatacaatggatgatatgaagaagtgtcaaatctttaacgaggatacgtcgtattatgttaaggaatacaatacatacctgtttttgcacgagtcggatcccatggtcatagagaacggaatactaaagaaactgtcatctataaaatccaagagtagacggctgaacttgtttagcaaaaacattttaaaatattatttagacggacaattggctcgtctaggtcttgtgttagatgattataaaggagacttgttagttaaaatgataaaccatcttaagtctgtggaggatgtatccgcattcgttcgattttctacagataaaaaccctagtattcttccatcgctaatcaaaactattttagctagttataatatttccatcatcgtcttatttcaaaggtttttaagagataatctatatcatgtagaagaattcttggataaaagcatccatctaaccaagacggataagaaatatatacttcaattgataagacacggtagatcatagaacagaccaaatatattattaataatttgtatatacatagatataattatcacatattaaaaattcacacatttttgataaatgggaactgctgcaacaattcagactcccaccaaattaatgaataaagaaaatgcagaaatgattttggaaaaaattgttgatcatatagttatgtatattagtgacgaatcaagtgattcagaaaataatcctgaatatattgattttcgtaacagatacgaagactatagatctctcattataaaaagtgatcacgagtttgtaaagctatgtaaaaatcatgcagagaaaagttctccagaaacgcaacaaatgattatcaaacacatatacgaacaatatcttattccagtatctgaagtactattaaaacctataatgtccatgggtgacataattacatataacggatgtaaagacaatgaatggatgctagaacaactctctaccctaaactttaacaatctccgcacatggaactcatgtagcataggcaatgtaacgcgtctgttttatacattttttagttatctgatgaaagataaactaaatatataagtataatcccattctaatactttaacctgatgtattagcatcttattagaatattaacctaactaaaagacataacataaaaactcattacatagttgataaaaagcggtaggatataaatattatggctgccaccgttccgcgttttgacgacgtgtacaaaaatgcacaaagaagaattctagatcaagaaacattttttagtagaggtctaagtagaccgttaatgaaaaacacatatctatttgataattacgcgtatggatggataccagaaactgcaatttggagtagtagatacgcaaacttagatgcaagtgactattatcccatttcgttgggattacttaaaaagttcgagtttctcatgtctctatataaaggtcctattccagtatacgaagaaaaagtaaatactgaattcattgctaatggatcgttctctggtagatacgtatcatatcttcgaaagttttctgctcttccaacaaacgagtttattagttttttgttactgacttccattccaatctataatatcttgttctggtttaaaaatactcagtttgatattactaaacacacattattcagatacgtctatacagataatgccaaacacctggcgttggctaggtatatgcatcaaacaggagactataagcctttgtttagtcgtctcaaagagaattatatatttaccggtcccgttccaataggtatcaaagatataaatcaccctaatcttagtagagcaagaagtccatccgattatgagacattagctaatattagtactatattgtactttaccaagtatgatccggtattaatgtttttattgttttacgtacctgggtattcaattactacaaaaattactccagccgtagaatatctaatggataaactgaatctaacaaagagcgacgtacaactgttgtaaattattttatgcttcgtaaaatgtaggttttgaaccaaacattctttcaaagaatgagatgcataaaactttattatccaatagattgactatttcggacgtcaatcgtttaaagtaaacttcgtaaaatattctttgatcactgccgagtttaaaacttctatcgataattgtttcatatgttttaatatttacaagttttttggtccatggtacattagccggacaaatatatgcaaaataatatcgttctccaagttctatagtttctggattatttttattatattcagtaaccaaatacatattagggttatctgcggatttataatttgagtgatgcattcgactcaacataaataattctagaggagacgatctactatcaaattcggatcgtaaatctgtttctaaagaacggagaatatctatacatacctgattagaattcatccgtccttcagacaacatctcagacagtctggtcttgtatgtcttaatcatattcttatgaaacttggaaacatctcttctagtttcactagtacctttattaattctctcaggtacagattttgaattcgacgatgccgagtatttcatcgttgtatatttcttcttcgattgcataatcagattcttatataccgcctcaaactctattttaaaattattaaacaatactctattattaatcagtcgttctaactcctttgctatttctatggacttatctacatcttgactgtctatctctgtaaacacggagtcggtatctccatacacgctacgaaaacgaaatctgtaatctataggcaacgatgttttcacaatcggattaatatctctatcgtccatataaaatggattacttaatggattggcaaaccgtaacataccgttagataactctgctccatttagtaccgattctagatacaagatcattctacgtcctatggatgtgcaactcttagccgaagcgtatgagtatagagcactatttctaaatcccatcagaccatatactgagttggctactatcttgtacgtatattgcatggaatcatagatggccttttcagttgaactggtagcctgttttaacatctttttatatctggctctctctgccaaaaatgttcttaatagtctaggaatggttccttctatcgatctatcgaaaattgctatttcagagatgaggttcggtagtctaggttcacaatgaaccgtaatatatctaggaggtggatatttctgaagcaagagctgattatttatttcttcttccaatctattggtactaacaacgacaccgactaatgtttccggagatagatttccaaagatacacacattaggatacagactgttataatcaaagattaatacattattactaaacattttttgttttggagcaaataccttaccgccttcataaggaaacttttgttttgtttctgatctaactaagatagttttagtttccaacaatagctttaacagtggacccttgatgactgtactcgctctatattcgaataccatggattgaggaagcacatatgttgacgcacccgcgtctgtttttgtttctactccataatactcccacaaatactgacacaaacaagcatcatgaatacagtatctagccatatctaaagctatgtttagattataatccttatacatctgagctaaatcaacgtcatcctttccgaaagataatttatatgtatcattaggtaaagtaggacataatagtacgactttaaatccattttcccaaatatctttacgaattactttacatataatatcctcatcaacagtcacataattacctgtggttaaaacctttgcaaatgcagcggctttgcctttcgcgtccgtagtatcgtcaccgatgaacgtcatttctctaactcctctatttaatactttacccatgcaactgaacgcgttcttggatatagaatccaatttgtacgaatccaatttttcagatttttgaatgaatgaatatagatcgaaaaatatagttccattattgttattaacgtgaaacgtagtattggccatgccgcctactcccttatgactagactgatttctctcataaatacagagatgtacagcttcctttttgtccggagatctaaagataatcttctctcctgttaataactctagacgattagtaatatatctcagatcaaagttatgtccgttaaaggtaacgacatagtcgaacgttagttccaacaattgtttagctattcgtaacaaaactatttcagaacataaaactagttctcgttcgtaatccatttccattagtgactgtatcctcaaacatcctctatcgacggcttcttgtatttcctgttccgttaacatctcttcattaatgagcgtaaacaataatcgtttaccacttaaatcgatataacagtaacttgtatgcgagattgggttaataaatacagaaggaaacttcttatcgaagtgacactctatatctagaaataagtacgatcttgggatatcgaatctaggtatttttttagcgaaacagttacgtggatcgtcacaatgataacatccattgttaatctttgtcaaatattgctcgtccaacgagtaacatccgtctggagatatcccgttagaaatataaaaccaactaatattgagaaattcatccatggtggcattttgtatgctgcgtttctttggctcttctatcaaccacatatctgcgacggagcattttctatctttaatatctagattataacttattgtctcgtcaatgtctatagttctcatctttcccaacggcctcgcattaaatggaggaggagacaatgactgatatatttcgtccgtcactacgtaataaaagtaatgaggaaatcgtataaatacggtctcaccatttcgacatctggatttcagatataaaaatctgttttcaccgtgactttcaaaccaattaatgcaccgaacatccatttatagaatttagaaatatattttcatttaaatgaatcccaaacattggggaagagccgtatggaccattatttttatagtactttcgcaagcgggtttagacggcaacatagaagcgtgtaaacgaaaactatatactatagttagcactcttccatgtcctgcatgtagacggcacgcgactatcgctatagaggacaataatgtcatgtctagcgatgatctgaattatatttattattttttcatcagattatttaacaatttggcatctgatcccaaatacgcgatcgatgtgacaaaggttaaccctttataaacttaacccattataaaacttatgattagtcacgactgaaataaccgcgtgattattttttggtataattctacacggcatggtttctgtgactatgaattcaacccccgttacattagtgaaatctttaacaaacagcaagggttcgtcaaagacataaaactcattgtttacaatcgaaatagaccccctatcacacttaaaataaaaaatatccttatcctttaccaccaaataaaattctgattggtcaatgtgaatgtattcacttaacagttccacaaatttatttattaactccgaggcacatacatcgtcggtattttttatggcaaactttactcttccagcatccgtttctaaaaaaatattaacgagttccatttatatcatccaatattattgaaatgacgttgatggacaaatgatacaaataagaaggtacggtacctttgtccaccatctcctccaattcatgctctattttgtcattaactttaatgtatgaaaacagtacgccacatgcttccatgacagtgtgtaacactttggatacaaaatgtttgacattagtataattgttcaagactgtcaatctataatagatagtagctataatatattctatgatggtattgaagaagatgacaaccttggcatattgatcatttaacacagacatggtatcaacagatagcttgaatgaaagagaatcagtaattggaataagcgtcttctcgatggagtgtccgtataccaacatgtctgatattttgatgtattccattaaattatttagttttttctttttattctcgttaaacagcatttctgtcaacggaccccaacatcgttgaccgattaagttttgattgatttttccgtgtaaggcgtatctagtcagatcgtatagcctatccaataatccatcgtctgtgtgtagatcacatcgtacactttttaattctctatagaagagcgacagacatctggagcaattacagacagcaatttctttattctctacagatgtaagatacttgaagacattcctatgatgatgcagaattttggataacacggtattgatggtatctgttaccataattcctttgatggctgatagtgtcagagcacaagatttccaatctttgacaatttttagcaccattatctttgttttgatatctatatcagacagcatggtgcgtctgacaacacaaggattaagacggaaagatgaaatgattctctcaacatcttcaatggataccttgctattttttctggcattatctatatgtgcgagaatatcctctagagaatcagtatcctttttgatgatagtggatctcaatgacatgggacgtctaaaccttcttattctatcaccagattgcatggtgatttgtcttctttcttttatcataatgtaatctctaaattcatcggcaaattgtctatatctaaaatcataatatgagatgtttacctctacaaatatctgttcgtccaatgttagagtatttacatcagttttgtattccaaattaaacatggcaacggatttaattttatattcctctattaagtcctcgtcgataataacagaatgtagataatcatttaatccatcgtacatggttggaagatgctcgttgacaaaatctttaattgtcttgatgaaggtgggactatatctaacatcttgattaataaaatttataacattgtccataggatactttgtaactagttttatacacatctcttcatcggtaagtttagacagaatatcgtgaacaggtggtatattatattcatcagatatacgaagaacaatgtccaaatctatattgtttaatatattatatagatgtagtgtagctcctacaggaatatctttaactaagtcaatgatttcatcaaccgttagatctattttaaagttaatcatataggcattgatttttaaaaggtatgtagccttgactacattctcattaattaaccattccaagtcactgtgtgtaagaagattatattctatcataagcttgactacatttggtcccgataccattaaagaattcttatgatataaggaaacagattttaggtactcatctactctacaagaattttggagagccttaacgatatcagtgacgtttattatttcaggaggaaaaaacctaacattgagaatatcggaattaatagcttccagatacagtgattttggcaatagtccgtgtaatccataatccagtaacacgagctggtgcttgctagacaccttttcaatgtttaatttttttgaaataagctttgataaagccttcctcgcaaattccggatacatgaacatgtcggcgacatgattaagtattgttttttcattatttttatattttctcaacaagttctcaataccccaatagatgatagaatatcacccaatgcgtccatgttgtctatttccaacaggtcgctatatccaccaatagaagttttcccaaaaaagattctaggaacagttctaccaccagtaatttgttcaaaatagtcacgcaattcattttcgggtttaaattctttaatatcgacaatttcatacgctcctcttttgaaactaaacttatttagaatatccagtgcatttctacaaaaaggacatgtatacttgacaaaaattgtcactttgttattggccaacctttgttgtacaaattcctcggccattttaatatttaagtgatataaaactatctcgacttatttaactctttagtcgagatatatggacgcagatagctatatgatagccaactacagaaggcaaacgctataaaaaacataattacgacgagcatatttataaatatttttattcagcattacttgatatagtaatattaggcacagtcaaacattcaaccactctcgatacattaactctctcattttctttaacaaattctgcaatatcttcgtaaaaagattcttgaaactttttagaatatctatcgactctagatgaaatagcgttcgtcaacatactatgttttgtatacataaaggcgcccattttaacagtttctagtgacaaaatgctagcgatcctaggatcctttagaatcacatagattgacgattcgtctctcttagtaactctagtaaaataatcatacaatctagtacgcgaaataatattatccttgacttgaggagatctaaacaatctagttttgagaacatcgataagttcatcgggaatgacatacatactatctttaatagaactcttttcatccagttgaatggattcgtccttaaccaactgattaatgagatcttctattttatcattttccagatgatatgtatgtccattaaagttaaattgtgtagcgcttctttttagtctagcagccaatactttaacatcactaatatcgatatacaaaggagatgatttatctatggtattaagaattcgtttttcgacatctgtcaaaaccaattcctttttgcctgtatcatccagttttccatcctttgtaaagaaattattttctactagactattaataagactgataaggattcctccataattgcacaatccaaactttttcacaaaactagactttacaagatctacaggaatgcgtacttcaggtttcttagcttgtgattttttcttttgtggacattttcttgtgaccaactcatctaccatttcattgattttagcagtgaaataagctttcaatgcacgggcactgatactattgaaaacgagttgatcttcaaattccgccatttaagttcaccaaacaacttttaaatacaaatatatcaatagtagtagaataagaactataaaaaaaataataattaaccaataccaaccccaacaaccggtattattagttgatgtgactgttttctcatcacttagaacagatttaacaatttctataaagtctgtcaaatcatcttccggagaccccataaatacaccaaatatagcggcgtacaacttatccatttatacattgaatattggcttttctttatcgctatcttcatcatattcatcatcaatatcaacaagtcccagattacgagccagatcttcttctacattttcagtcattgatacacgttcactatctccagagagtccgataacgttagccaccacttctctatcaatgattagtttcttgagtgcgaatgtaatttttgtttccgttccggatctatagaaaactacaggtgtgataattgccttggccaattgtctttctcttttactgagtgattctagttcaccttctatagatctgagaatggatgattctccagtcgaaacatattctaccatggctccgtttaatttgttgatgaagatggattcatccttaaatgttttctctgtaatagtttccaccgaaagactatgcaaagaatttggaatgcgttccttgtgcttaatgtttccatagacggcttctagaagttgatacaacataggactagccgcggtaacttttatttttagaaagtatccatcgcttctatcttgtttagatttatttttataaagtttagtctctccttccaacataataaaagtggaagtcatttgactagataaactatcagtaagttttatagagatagacgaacaattagcgtattgagaagcatttagtgtaacgtattcgatacattttgcattagatttactaatcgattttgcatactctataacacccgcacaagtctgtagagaatcgctagatgcagtaggtcttggtgaagtttcaactctcttcttgattaccttactcatgattaaacctaaataattgtactttgtaatataatgatatatattttcactttatctcatttgagaataaaaatgtttttgtttaaccactgcatgatgtacagatttcggaatcacaaaccaccggtggttttattttatccttgtccaatgtgaattgaatgggagcggatgcgggtttcgtacgtagatagtacattcccgtttttagaccgagactccatccgtaaaaatgcatactcgttagtttggaataactcggatctgctatatggatattcatagattgactttgatcgatgaaggctcccctgtctgcagccatttttatgatcgtcttttgtggaatttcccaaatagttttataaactcgcttaatatcttctggaaggtttgtattctgaatggatccaccatctgccataatcctattcttgatctcatcattccataattttctctcggttaaaactctaaggagatgcggattaactacttgaaattctccagacaatactctccgagtgtaaatattactggtatacggttccaccgactcattatttcccaaaatttgagcagttgatgcagtcggcataggtgccaccaataaactatttctaagaccgtatgttctgattttatcttttagaggttcccaattccaaagatccgacggtacaacattccaaagatcatattgtagaataccgttactggcgtacgatcctacatatgtatcgtatggtccttccttctcagctagttcacaactcgcctctaatgcaccgtaataaatggtttcgaagatcttcttatttagatcttgtgcttccaggctatcaaatggataatttaagagaataaacgcgtccgctaatccttgaacaccaataccgataggtctatgtctcttattagagatttcagcttctggaataggataataattaatatctataattttattgagatttctgacaattactttgaccacatccttcagtttgagaaaatcaaatcgcccatctattacaaacatgttcaaggcaacagatgccagattacaaacggctacctcattagcatccgcatattgtattatctcagtgcaaagattactacacttgatagttcctaaattttgttgattactctttttgttacacgcatccttataaagaatgaatggagtaccagtttcaatctgagattctataatcgctttccagacgactcgagcctttattatagatttgtatctcctttctctttcgtatagtgtatacaatcgttcgaactcgtctccccaaacattgtccaatccaggacattcatccggacacatcaacgaccactctccgtcatccttcactcgtttcataaagagatcaggaatccaaagagctataaatagatctctggttctatgttcctcgtttcctgtattctttttaagatcgaggaacgccataatatcagaatgccacggttccaagtatatggccataactccaggccgtttgtttcctccctgatctatgtatctagcggtgttattataaactctcaacattggaataataccgtttgatataccattggtaccggagatatagcttccactggcacgaatattactaattgatagacctattccccctgccattttagagattaatgcgcatcgttttaacgtgtcatagataccctctatgctatcatcgatcatgttaagtagaaaacagctagacatttggtgacgactagttcccgcattaaataaggtaggagaagcgtgcgtaaaccatttttcagaaagtagattgtacgtctcaatagctgagtctatatcccattgatgaattcctactgcgacacgcattaacatgtgctgaggtctttcaacgatcttgttgtttattttcaacaagtaggatttttccaaagttttaaaaccaaaatagttgtatgaaaagtctcgttcgtaaataataaccgagttgagtttatccttatatttgttaactatatccatggtgatacttgaaataatcggagaatgtttcccatttttaggattaacatagttgaataaatcctccatcacttcactaaatagtttttttgtttccttgtgtagatttgatacggctattctggcggctagaatggcataatccggatgttgtgtagtacaagtggctgctatttcggctgccagagtgtccaattctaccgttgttactccattatatattccttgaataaccttcatagctattttaataggatctatatgatccgtgtttaagccataacataattttctaatacgagacgtgattttatcaaacatgacattttccttgtatccatttcgtttaatgacaaacatttttgttggtgtaataaaaaaattatttaacttttcattaatagggatttgacgtacgtagcgtacaaaatgattgttcctggtatatagataaagagtcctatatatttgaaaatcgttacggctcgattaaactttaatgattgcatagtgaatatatcattaggatttaactccttgactatcagggcggcaccagaaattaccatcaaaagcattaatacagttatgcctatcgcagttagaacggttatagcatccaccatttatatctaaaaattagatcaaagaatatgtgacaaagtcctagttgtatattgagaattgacaaaacaatgtttcttacatattttttttttattagtaaccgacttaatagtaggaactggaaaactagacttgattattctataagtatagatacccttccaaataatattctctttgataaaagttccagaaaatgtagaattttttaaaaagttatcttttgctattaccaagattgtgtttagacgcttattattaatatgagtgatgaaatccacaccgcctctagatatcgcctttatttccacattagatggtaaatccaatagtgaaactatctttttaggaatgtatggactcgcgtttagaggagtgaacgtcttgggcgtcggaaaggatgattcgtcaaacgaataaacaatttcacaaatggatgttaatgtattagtaggaaattttttgacgctagtggaattgaaaattctaatggatgatgttctacctatttcatccgataacatgttaatttccgacaccaacggttttaatatttcgatgatatacggtagtctctctttcggacttatatagcttattccacaatacgagtcattatatactccaaaaaacaaaataactagtataaaatctgtatcgaatgggaaaaacgaaattatcgacataggtatagaatccggaacattgaacgtattaatacttaattctttttctgtggtaagtaccgataggttattgacattgtatggttttaaatattctataacttgagacttgatagatattagtgatgaattgaaaattatttttatcaccacgtgtgtttcaggatcatcgtcgacgcccgtcaaccaaccgaacggagtaaaataaatatcattaatatatgctctagatattagtatttttatcaatcctttgattatcatcttctcgtaggcgaatgattccatgatcaagagtgatttaagaacatcctccggagtattaatgggcttagtaaacagtccatcgttgcaataataaaagttatccaagttaaaggatattatgcattcgtttaaagatatcacctcatctgacggagacaattttttggtaggttttagagactttgaagctacttgtttaacaaagttattcatcgtcgtttactattctatttaattttgtagttaatttatcacatatcacattaattgactttttggtccatttttccatacgtttatattcttttaatcctgcgttatccgtttccgttatatccagtgatagatcgtgcaggttaaatagaatgctcttaaataatgtcatttttttatccgctaaaaatttaaagaatgtataaacttttttcaaagatttaaaacttttaggtggtgtcctagtacacaatatcataaacaaactaataaacatcccgcattcagattccaacagctgattaacttccacattaatacagcctattttcgctccaaatgtacattcgaaaaatctgaataaaacatcaatgtcgcaatttgtattatccaatacagaatgtttgtgattcgtgttaaaaccatcggagaaggaatagaaataaaaattattatagtggtggaattcagttggaatattgcctccggagtcataaaaggatactaaacattgttttttatcataaattacacatttccaatgagacaaataacaaaatccaaacattacaaatctagaggtagaacttttaattttgtctttaagtatatacgataagatatgtttattcataaacgcgtcaaatttttcatgaatcgctaaggagtttaagaatctcatgtcaaattgtcctatataatccacttcggatccataagcaaactgagagactaagttcttaatacttcgattgctcatccaggctcctctctcaggctctattttcatcttgacgacctttggattttcaccagtatgtattcctttacgtgataaatcatcgattttcaaatccatttgtgagaagtctatcgccttagatactttttcccgtagtcgaggtttaaagaaatacgctaacggtatactagtaggtaactcaaaaacatcatatatagaatggtaacgcgtctttaactcgtcggttaactctttcttttgatcgagttcgtcgctactattgggtctgctcaggtgccccgactctactagttccaacatcataccgataggaatacaagacactttgccggcggttgtagatttatcatatttttccactacatatccgttacaatttgttaaaaatttagatacatctatattgctacataatccagctagtgaatatatatgacataataaattggtaaatcctagttctggtattttactaattactaaatctgtatatctttccatttatcatggaaaagaatttaccagatatcttcttttttccaaactgcgttaatgtattctcttacaaatattcacaagatgaattcagtaatatgagtaaaacggaacgtgatagtttctcattggccgtgtttccagttataaaacatagatggcataacgcacacgttgtaaaacataaaggaatatacaaagttagtacagaagcacgtggaaaaaaagtatctcctccatcactaggaaaacccgcacacataaacctaaccgcgaagcaatatatatacagtgaacacacaataagctttgaatgttatagttttctaaaatgtataacaaatacagaaatcaattcgttcgatgagtatatattaagaggactattagaagctggtaatagtttacagatattttccaattccgtaggtaaacgaacagatactataggtgtactagggaataagtatccatttagcaaaattccattggcctcattaactcctaaagcacaacgagagatattttcagcgtggatttctcatagacctgtagttttaactggaggaactggagtgggtaagacgtcacaggtacccaagttattgctttggtttaattatttatttggtggattctctactctagataaaatcactaactttcacgaaagaccagtcattctatctcttcctaggatagctttagttagattgcatagcaataccattttaaaatcattgggatttaaggtactagatggatctcctatttctttacggtacggatctataccggaagaattaataaacaaacaaccaaaaaaatatggaattgtattttctacccataagttatctctaacaaaactatttagttatggcactcttattatagacgaagttcatgagcatgatcaaataggagatattattatagcagtagcgagaaagcatcatacgaaaatagattctatgtttttaatgactgccacgttagaggatgaccgagaacggctaaaagtatttttacctaatcccgcatttatacatattcctggagatacactgtttaaaattagcgaggtatttattcataataagataaatccatcttccagaatggcatacatagaagaagaaaagagaaatttagttactgctatacagatgtatactcctcctgatggatcatccggtatagtctttgtggcatccgttgcacagtgtcacgaatataaatcatatttagaaaaaagattaccgtatgatatgtatattattcatggtaaggtcttagatatagacgaaatattagaaaaagtgtattcatcacctaatgtatcgataattatttctactccttatttggaatccagcgttactatacgcaatgttacacacatttatgatatgggtaaagtttttgtccccgctccttttggaggatcgcaagaatttatttctaaatctatgagagatcaacgaaaaggaagagtaggaagagttaatcctggtacatacgtctatttctatgatctgtcttatatgaagtctatacagcgaatagattcagaatttctacataattatatattgtacgctaataagtttaatctaacactccccgaagatttgtttataatccctacaaatttggatattctatggcgtacaaaggaatatatagactcgttcgatattagtacagaaacatggaataaattattatccaattattatatgaagatgatagagtatgctaaactttatgtactaagtcctattctcgctgaggagttggataactttgagaggacgggagaattaactagtattgtacgagaagccattttatctctaaatttacgaattaagattttaaattttaaacataaagatgatgatacgtatatacacttttgtaaaatattattcggtgtctataacggaacaaacgctactatatattatcatagacctctaacgggatatatgaatatgatttcagatactatatttgttcctgtagataataactaaaaatcaaactctaatgaccacatctttttttagagatgaaaaattttctacatctccttttgtagacacgactaaacattttgcaaaaaaaagtttattagtgtttagataatcgtatacttcatcagtgtagatagtaaatgtgaacaaataaaaggtattcttactcaatagattggtaaattccatagaatatattaatcctttcttcttgagatcccacatcatttcaaccagagacgttttatccaatgatttacctcgtactataccacatacaaaactagattttgcagtgacgtcgtacctggtattcctaccaaacaaaattttacttttagttcttttagaaaattctaaggtagaatctctatttgccaatatgtcatctatggaattaccactagcaaaaaatgatagaaatatatattgatacatcgcagctggttttgatctactatactttaaaaacgaatcagattccataattgcctgtatatcatcagctgaaaaactatgttttacacgtattccttcggcatttctttttaatgatatatcttgtttagacaatgataaagttatcatgtccatgagagacgcgtctccgtatcgtataaatatttcattagatgttagacgcttcattaggggtatacttctataaggtttcttaatcagtccatcattggttgcgtcaagaactactatcggatgttgttgggtatctctagtgttacacatggccttactaaagtttgggtaaataactatgatatctctattaattatagatgcatatatttcattcgtcaaggatattagtatcgacttgctatcgtcattaatacgtgtaatgtaatcatataaatcatgcgatagccaaggaaaattcaaatagatgttcatcatataatcgtcgctataattcatattaatacgttgacattgactaatttgtaatatagcctcgccacgaagaaagctctcgtattcagtttcatcgataaaggataccgttaaatataactggttgccgatagtctcatagtctattaagtggtaagtttcgtacaaatacagaatccctaaaatattatctaatgttggattaatctttaccataactgtataaaatggagacggagtcataactattttaccgtttgtacttactggaatagacgaaggaataatctccggacatgctggtaaagacccaaatgtctgtttgaagaaatccaatgttccaggtcctaatctcttaacaaaaattacgatattcgatcccgatatcctttgcattctatttaccagcatatcacgaactatattaagattatctatcatgtctattctcccaccgttatataaatcgcctccgctaagaaacgttagtatatccatacaatggaatacttcatttctaaaatagtattcgttttctaattctttaatgtgaaatcgtatactagaaagggaaaaattatctttgagttttccgttagaaaagaaccacgaaactaatgttctgattgcgtccgattccgttgctgaattaatggatttacaccaaaaactcatataacttctagatgtagaagcattcgctaaaaaattagtagaatcaaaggatataagtagatgttccaacaagtgagcaattcccaagatttcatctatatcattctcgaatccgaaattagaaattcccaagtagatatcctttttcatccgatcgttgatgaaaatacgaactttattcggtaagacaatcatttactaaggagtaaaataggaagtaatgttcgtatgtcgttatcatcgtataaattaaaggtgtgttttttaccattaagtgacattataattttaccaatattggaattataatataggtgtatttgcgcactcgcgacggttgatgcatcggtaaatatagctgtatctaatgttctagtcggtatttcatcatttcgctgtctaataatagcgttttctctatctgtttccattacagctgcctgaagtttattggtcggataatatgtaaaataataagaaatacatacgaataacaaaaataaaataagatataataaagatgccatttagagatctaattttgttcaacttgtccaaattcctacttacagaagatgaggaatcgttggagatagtgtcttccttatgtagaggatttgaaatatcttatgatgacttgataacttactttccagataggaaataccataaatatatttctaaagtatttgaacatgtagatttatcggaggaattaagtatggaattccatgatacaactttgcgagatttagtctatcttagattgtacaagtattccaagtgtatacggccgtgttataaattaggagataatctaaaaggcatagttgttataaaggacaggaatatttatattagggaagcaaatgatgacttgatagaatatctcctcaaggaatacactcctcagatttatacatattctaatgagcgcgtccccataactggttcaaaattaattctttgtggattttctcaagttacatttatggcgtatacaacgtcgcatataacaacaaataaaaaggtagatgttctcgtttccaaaaaatgtatagatgaactagtcgatccaataaattatcaaatacttcaaaatttatttgataaaggaagcggaacaataaacaaaatactcaggaagatattttattcggtaacaggtggccaaactccataggtagctttttctatttcggattttagaatttccaaattcaccagcgatttatctgttttggtgaaatccaaggatttattaatgtccacaaatgccatttgttttgtctgtggattgtatttgaaaatggaaacgatgtagttagatagatgcgctgcgaagtttcctattagggttccgcgcttcacgtcacccagcatacttgaatcaccatcctttaaaaaaaatgataagatatcaacatggagtatatcatactcggattttaattcttctactgcatcactgacattttcacaaatactacaatacggtttaccgaaaataatcagtacgttcttcatttatgggtatcaaaaacttaaaatcgttactgctggaaaataaatcactgacgatattagatgataatttatacaaagtatacaatggaatatttgtggatacaatgagtatttatatagccgtcgccaattgtgtcagaaacttagaagagttaactacggtattcataaaatacgtaaacggatgggtaaaaaagggagggcatgtaaccctttttatcgatagaggaagtataaaaattaaacaagacgttagagacaagagacgtaaatattctaaattaaccaaggacagaaaaatgctagaattagaaaagtgtacatccgaaatacaaaatgttaccggatttatggaagaagaaataaaggcagaaatgcaattaaaaatcgataaactcacatttcaaatatatttatctgattctgataacataaaaatatcattgaatgagatactaacacatttcaacaataatgagaatgttacattattttattgtgatgaacgagacgcagaattcgttatgtgtctcgaggctaaaacacatttctctaccacaggagaatggccgttgataataagtaccgatcaggatactatgctatttgcatctgctgataatcatcctaagatgataaaaaacttaactcaactgtttaaatatgttccatctgcagaggataactatttagcaaaattaacggcgttagtgaatggatgtgatttctttcctggactctatggggcatctataacacccaacaacttaaacaaaatacaattgtttagtgattttacaatcgataatatagtcactagtttggcaattaaaaattattatagaaagactaactctaccgtagacgtgcgtaatattgttacgtttataaacgattacgctaatttagacgatgtctactcgtatattcctccttgtcaatgcactgttcaagaatttatattttccgcattagatgaaaaatggaatgaatttaaatcatcttatttagaaagcgtgccgttaccctgccaattaatgtatgcgttagaaccacgcaaggagattgatgtttcagaagttaaaactttatcatcttatatagatttcgaaaatactaaatcagatatcgatgttataaaatctatatcctcgatcttcggatattctaacgaaaactgtaacacgatagtattcggcatctataaggataatttactactgagtataaatagttcattttactttaacgatagtctgttaataaccaatactaaaagtgataatataataaatataggttactagattaaaaatggtgttccaactcgtgtgctctacgtgcggcaaagatatttctcacgaacgatataaattgattatacgaaaaaaatcattaaaggatgtactcgtcagtgtaaagaacgaatgttgtaggttaaaattatctacacaaatagaacctcaacgtaacttaacagtgcaacctctattggatataaactaatatggatccggttaattttatcaagacatatgcgcctagaggttctattatttttattaattataccatgtcattaacaagtcatttgaatccatcgatagaaaaacatgtgggtatttattatggtacgttattatcggaacacttggtagttgaatcaacatatagaaaaggagttcgaatagtcccattggatagtttttttgaaggatatcttagtgcaaaagtatacatgttagagaatattcaagttatgaaaatagcagctgatacgtcattaactttattgggtattccgtatggatttggtcatgatagaatgtattgttttaaattggtagctgactgttataaaaatgccggtattgatacatcgtctaaacgaatattaggtaaagatatttttctgagccaaaacttcacagacgataatagatggataaagatatatgattctaataatttaacattttggcaaattgattaccttaaagggtgagttaatatgcataactactcctccgttgttttttccctcgttctttttcttaacgttgtttgccatcactctcataatgtaaagatattctaaaatggtaaacttttgcatatcggacgcagaaattggtataaatgttgtaattgtattatttcccgtcaatggactagtcacagctccatcagttttatatcctttagagtatttctcactcgtgtctaacattctagagcattccatgatctgtttatcgttgatattggccggaaagatagattttttattttttattatattactattggcaattgtagatataacttctggtaaatatttttctaccttttcaatctcttctattttcaagccggctatatattctgctatattgttgctagtatcaataccttttctggctaagaagtcatatgtggtattcactatatcagttttaactggtagttccattagcctttccacttctgcagaataatcagaaattggttctttaccagaaaatccagctactataataggctcaccgatgatcattggcaaaatcctatattgtaccagattaatgagagcatatttcatttccaataattctgctagttcttgagacattgatttatttgatgaatctagttggttctctagatactctaccatttctgccgcatacaataacttgttagataaaatcagggttatcaaagtgtttagcgtggctagaatagtgggcttgcatgtattaaagaatgcggtagtatgagtaaaccgttttaacgaattatatagtctccagaaatctgtggcgttacatacatgagccgaatgacatcgaagattgtccaatatttttaatagctgctctttgtccattatttctatatttgactcgcaacaattgtagataccattaatcaccgattcctttttcgatgccggacaatagcacaattgtttagctttggactctatgtattcagaattaatagatatatctcttaatacagattgcactatacattttgaaactatgtcaaaaattgtagaacgacgctgttctgcagccatttaactttaaataatttacaaaaatttaaaatgagcatccgtataaaaatcgataaactgcgccaaattgtggcatatttttcagagttcagtgaagaagtatctataaatgtagactcgacggatgagttaatgtatatttttgccgccttgggcggatctgtaaacatttgggccattatacctctcagtgcatcagtgttctaccgcggagccgaaaatattgtgtttaatcttcctgtgtccaaggtaaaatcgtgtttgtgtagttttcacaatgatgccatcatagatatagaacctgatctggaaaataatctagtaaaactttctagttatcatgtagtaagtgtcgattgtaataaggaactgatgcctattaggacagatactactatttgtctaagtatagatcaaaagaaatcttacgtgtttaattttcacaagtatgaagaaaaatgttgtggtagaaccgtcattcatttagaatggttgttgggctttatcaagtgtattagtcagcatcagcatttggctattatgtttaaagatgacaatattattatgaagactcctggtaatactgatgcattttccagggaatattctatgactgaatgttctcaagaactacaaaagttttctttcaaaatagctatctcgtctctcaacaaactacgaggattcaaaaagagagtcaatgtttttgaaactagaatcgtaatggataatgacgataacattttaggaatgttgttttcggatagagttcaatcctttaagatcaacatctttatggcgtttttagattaatactttcaatgagataaatatgggtggcggagtaagtgttgagctccctaaacgggatccgcctccgggagtacccactgatgagatgttattaaacgtggataaaatgcatgacgtgatagctcccgctaagcttttagaatatgtgcatataggaccactagcaaaagataaagaggataaagtaaagaaaagatatccagagtttagattagtcaacacaggacccggtggtctttcggcattgttaagacaatcgtataatggaaccgcacccaattgctgtcgcacttttaatcgtactcattattggaagaaggatggaaagatatcagataagtatgaagagggtgcagtattagaatcgtgttggccagacgttcacgacaccggaaaatgcgatgttgatttattcgactggtgtcagggggatacgttcgatagaaacatatgccatcagtggatcggttcagcctttaataggagtaatagaactgtagagggtcaacaatcgttaataaatctgtataataagatgcaaacattatgtagtaaagatgctagtgtaccaatatgtgaatcatttttgcatcatttacgcgcacacaatacagaagatagcaaagagatgatcgattatattctaagacaacagtctgcggactttaaacagaaatatatgagatgtagttatcccactagagataagttagaagagtcattaaaatatgcggaacctcgagaatgttgggatccagagtgttcgaatgccaatgttaatttcttgctaacacgtaattataataatttaggactttgcaatattgtacgatgtaatactagcgtgaacaacttacagatggataaaacttcctcattaagattgtcatgtggattaagcaatagtgatagattttctactgttcccgtcaatagagcaaaagtagttcaacataatattaaacactcgttcgacctaaaattgcatttgatcagtttattatctctcttggtaatatggatactaattgtagctatttaaatgggtgccgcggcaagcatacagacgacggtgaatacactcagcgaacgtatctcgtctaaattagaacaagaagcgaatgctagtgctcaaacaaaatgtgatatagaaatcggaaatttttatatccgacaaaaccatggatgtaacctcactgttaaaaatatgtgctctgcggacgcggatgctcagttggatgctgtgttatcagccgctacagaaacatatagtggattaacaccggaacaaaaagcatacgtgccagctatgtttactgctgcgttaaacattcagacgagtgtaaacactgttgttagagattttgaaaattatgtgaaacagacttgtaattctagcgcggtcgtcgataacaaattaaagatacaaaacgtaatcatagatgaatgttacggagccccaggatctccaacaaatttggaatttattaatacaggatctagcaaaggaaattgtgccattaaggcgttgatgcaattgacgactaaggccactactcaaatagcacctaaacaagttgctggtacaggagttcagttttatatgattgttatcggtgttataatattggcagcgttgtttatgtactatgccaagcgtatgttgttcacatccaccaatgataaaatcaaacttattttagccaataaggaaaacgtccattggactacttacatggacacattctttagaacttctccgatggttattgctaccacggatatgcaaaactgaaaatatattgataatattttaatagattaacatggaagttatcactgatcgtctagacgatatagtgaaacaaaatatagcggatgaaaaatttgtagattttgttatacacggtctagagcatcaatgtcctgctatacttcgaccattaattaggttgtttattgatatactattatttgttatagtaatttatatttttacggtacgtctagtaagtagaaattatcaaatgttgttggcgttggtggcgctagtcatcacattaactattttttattactttatactataatagtactagactgacttctaacaaacatctcacctgccataaataaatgcttgatattaaagtcttctatttctaacactattccatctgtggaaaataatactctgacattatcgctaattgacacatcggtgagtgatatgcctataaagtaataatcttctttgggcacatataccagtgtaccaggttctaacaacctatttactggtgctcctatagcatactttttctttaccttgagaatatccatcgtttgcttggtcaatagcgatatgtgattttttatcaaccactcgaaaaagtaattggagtgttcatatcctctacgggctattgtctcatggccgtgtatgaaatttaagtaacacgactgtggtagatttgttctatagagccggttgccgcaaatagatagaactaccaatatgtctgtacaaatgttaaacattaattgattaacagaaaaaacaatgttcgttctgggaatagaaaccagatcaaaacaaaattcgttagaatatatgccacgtttatacattgaatataaaataactacagtttgaaaaataacagtatcatttaaacatttaacttgcggggttaatctcacaactttactgtttttgaactgttcaaaatatagcatagatccgtgagaaatacgtttagccgcctttaatagaggaaatcccaccgcctttctggatctcaccaacgacgatagttctgaccagcaactcatttcttcatcatccacctgttttaacatataataggcaggagatagatatccgtcattgcaatattccttctcgtaggcacacaatctaatattgataaaatctccattctcttctctgcatttattatcttgtttcggtggctgattaggctgtagtcttggtttaggctttggtatatcgttgttgaatctattttggtcattaaatctttcatttcttcctggtatatttttatcacctcgtttggttggatttttgtctatattatcgtttgtaacatcggtacgggtattcatttatcacaaaaaaaacttctctaaatgagtctactgctagaaaacctcatcgaagaagataccatattttttgcaggaagtatatctgagtatgatgatttacaaatggttattgccggcgcaaaatccaaatttccaagatctatgctttctatttttaatatagtacctagaacgatgtcaaaatatgagttggagttgattcataacgaaaatatcacaggagcaatgtttaccacaatgtataatataagaaacaatttgggtctaggagatgataaactaactattgaagccattgaaaactatttcttggatcctaacaatgaagttatgcctcttattattaataatacggatatgactgccgtcattcctaaaaaaagtggtaggagaaagaataagaacatggttatcttccgtcaaggatcatcacctatcttgtgtattttcgaaactcgtaaaaagattaatatttataaagaaaatatggaatccgcgtcgactgagtatacacctatcggagacaacaaggctttgatatctaaatatgcgggaattaatatcctaaatgtgtattctccttccacatccataagattgaatgccatttacggattcaccaataaaaataaactagagaaacttagtactaataaggaactagaatcgtatagttctagccctcttcaagaacccattaggttaaatgattttctgggactattggaatgtgttaaaaagaatattcctctaacagatattccgacaaaggattgattactataaatggagaatgttcctaatgtatactttaatcctgtgtttatagagcccacgtttaaacattctttattaagtgtttataaacacagattaatagttttatttgaagtattcgttgtattcattctaatatatgtattttttagatctgaattaaatatgttcttcatgcctaaacgaaaaatacccgatcctattgatagattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctttattgtcatcatgaacggcggacatattcagttgataatcggccccatgttttcaggtaaaagtacagaattaattagacgagttagacgttatcaaatagctcaatataaatgcgtgactataaaatattctaacgataatagatacggaacgggactatggacgcatgataagaataattttgaagcattggaagcaactaaactatgtgatgtcttggaatcaattacagatttctccgtgataggtatcgatgaaggacagttctttccagacattgttgaattctgtgagcgtatggcaaacgaaggaaaaatagttatagtagccgcactcgatgggacatttcaacgtaaaccgtttaataatattttgaatcttattccattatctgaaatggtggtaaaactaactgctgtgtgtatgaaatgctttaaggaggcttccttttctaaacgattgggtgaggaaaccgagatagaaataataggaggtaatgatatgtatcaatcggtgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgagattattaagtatttataccggtgagaacatgagactgactcgagttaccaaatcagacgctgtaaattatgaaaaaaagatgtactaccttaataagatcgtccgtaacaaagtagttgttaactttgattatcctaatcaggaatatgactattttcacatgtactttatgctgaggaccgtgtactgcaataaaacatttcctactactaaagcaaaggtactatttctacaacaatctatatttcgtttcttaaatattccaacaacatcaactgaaaaagttagtcatgaaccaatacaacgtaaaatatctagcaaaaattctatgtctaaaaacagaaatagcaagagatccgtacgcagtaataaatagaaacgtactactgagatatactaccgatatagagtataatgatttagttactttaataaccgttagacataaaattgattctatgaaaactgtgtttcaggtatttaacgaatcatccataaattatactccggttgatgatgattatggagaaccaatcattataacatcgtatcttcaaaaaggtcataacaagtttcctgtaaattttctatacatagatgtggtaatatctgacttatttcctagctttgttagactagatactacagaaactaatatagttaatagtgtactacaaacaggcgatggtaaaaagactcttcgtcttcccaaaatgttagagacggaaatagttgtcaagattctctatcgccctaatataccattaaaaattgttagatttttccgcaataacatggtaactggagtagagatagccgatagatctgttatttcagtcgctgattaatcaattagtagagatgagataagaacattataataatcaataatatatcttatatctcgtttagaaaaatgctaatattaaaatagctaacgctagtaatccaatcggaagccatttgatatctataatagggtatctaatttcctgattcagatagcggacagctatattctcggtagctactcgtttggaatcacaaacattatttacatctaatttactatctgtaatggaaacgtttcccaatgaaatggtacaatccgatacattgcattttgttatatttttttttaaagaggctggtaacaacgcatcgcttcgtttacatggctcgtaccaacaataatagggtaatcttgtatctattcctatccgtactatgcttttatcaggataaatacatttacatcgtatatcgtctttgttagcatcacagaatgcataaatttgttcgtccgtcatgataaaaatttaaagtgtaaatataactattatttttatagttgtaataaaaagggaaatttgattgtataccttcggttctttaaaagaaactgacttgataaaaatggctgtaatctctaaggttacgtatagtctatatgatcaaaaagagattaatgctacagatattatcattagtcatgttaaaaatgacgacgatatcggtaccgttaaagatggtagactaggtgctatggatggggcattatgtaagacttgtgggaaaacggaattggaatgtttcggtcactggggtaaagtaagtatttataaaactcatatagttaagcctgaatttatttcagaaattattcgtttactgaatcatatatgtattcactgcggattattgcgttcacgagaaccgtattccgacgatattaacctaaaagagttatcgggacacgctcttaggagattaaaggataaaatattatccaagaaaaagtcatgttggaacagtgaatgtatgcaaccgtatcaaaaaattactttttcaaagaaaaaggtttgtttcgtcaacaagttggatgatattaacgttcctaattctctcatctatcaaaagttaatttctattcatgaaaagttttggccattattagaaattcatcaatatccagctaacttattttatacagactactttcccatccctccgttgattattagaccggctattagtttttggatagatagtatacccaaagaaaccaatgaattaacttacttattaggtatgatcgttaagaattgtaacttgaatgctgatgaacaggttatccagaaggcggtaatagaatacgatgatattaaaattatttctaataacacttccagtatcaatttatcatatattacatccggcaaaaataatatgattagaagttatatcgtcgcccgacgaaaagatcagacggctagatctgtaattggtcccagtacatctatcaccgttaatgaggtaggaatgcccgcatatattagaaatacacttacagaaaagatatttgttaatgcctttacagtggataaagttaaacaactattagcatcaaaccaagttaaattttactttaataaacgattaaaccaattaacaagaatacgccaaggaaagtttatcaaaaataaaatacatttattgcctggtgattgggtagaagtagctgttcaagaatatacaagtattatttttggaagacagccgtctctacatagatacaacgtcatcgcttcatctatcagagctaccgaaggagatactatcaaaatatctcccggaattgccaactctcaaaatgctgatttcgacggagatgaagaatggatgatattagaacaaaatcctaaagctgtaattgaacaaagtattcttatgtatccgacgacgttactcaaacacgatattcatggagcccccgtttatggatctattcaagatgaaatcgtagcagcgtattcattgtttaggatacaagatctttgtttagatgaagtattgaacatcttggggaaatatggaagagagttcgatcctaaaggtaaatgtaaattcagcggtaaagatatctatacttacttgataggtgaaaagattaattatccgggtctcttaaaggatggtgaaattattgcaaacgacgtagatagtaattttgttgtggctatgaggcatctgtcattggctggactcttatccgatcataagtcgaacgtggaaggtatcaactttattatcaagtcatcttatgtttttaagagatatctatctatttacggttttggggtgacattcaaagatctgagaccaaattcgacgttcactaataaattggaggccatcaacgtagaaaaaatagaacttatcaaagaagcatacgccaaatatctcaacgatgtaagagacgggaaaatagttccattatctaaagctttagaggcggactatgtggaatccatgttatccaacttgacaaatcttaatatccgagagatagaagaacatatgagacaaacgctgatagatgatccagataataacctcctgaaaatggccaaagcgggttataaagtaaatcctacagaactaatgtatattctaggtacgtatggacaacaaaggattgatggtgaaccagcagagactcgagtattgggtagagtcttaccttactatcttccagactctaaggatccagaaggaagaggttacattcttaattctttaacaaaaggattaacaggttctcaatattacttttcgatgctggttgcaagatctcaatctactgatatcgtctgtgaaacatcacgtaccggaacactggctagaaaaatcattaaaaagatggaggatatggtggtcgacggatacggacaagtagttataggtaatacgctcatcaagtacgccgccaattataccaaaattctaggctcagtatgtaaacctgtagatcttatctatccagatgagtccatgacttggtatttggaaattagtgctctgtggaataaaataaaacagggattcgtttactctcagaaacagaaacttgcaaagaagacattggcgccgtttaatttcctagtattcgtcaaacccaccactgaggataatgctattaaggttaaggatctgtacgatatgattcataacgtcattgatgatgtgagagagaaatacttctttacggtatctaatatagattttatggagtatatattcttgacgcatcttaatccttctagaattagaattacaaaagaaacggctatcactatctttgaaaagttctatgaaaaactcaattatactctaggtggtggaactcctattggaattatttctgcacaggtattgtctgagaagtttacacaacaagccctgtccagttttcacactactgaaaaaagtggtgccgtcaaacaaaaacttggtttcaacgagtttaataacttgactaatttgagtaagaataagaccgaaattatcactctggtatccgatgatatctctaaacttcaatctgttaagattaatttcgaatttgtatgtttgggagaattaaatccagacatcactcttcgaaaagaaacagataggtatgtagtagatataatagtcaatagattatacatcaagagagcagaaattaccgaattagtcgtcgaatatatgattgaacgattcatctcctttagcgtcattgtaaaggaatggggtatggaaacattcattgaggacgaggataatattagatttactgtctatctaaatttcgttgaaccagaggaattgaatcttagtaagtttatgatggttcttccgggggcagccaacaagggaaagattagtaaattcaagattcctatctctgattatacgggttatgacgacttcaatcaaacaaaaaagctcaataagatgactgtagaactcatgaatctaaaagaattaggttctttcgatttggaaaacgtcaacgtgtatcctggagtatggaatacatacgatatcttcggtatcgaggccgctcgtgaatacttgtgcgaagccatgttaaacacctatggagaagggttcgattatctgtatcagccttgtgatcttctcgctagtttactatgtgctagttacgaaccagaatcagtgaataaattcaagttcggcgcagctagtactcttaagagagctacgttcggagacaataaagcattgttaaacgcggctcttcataaaaagtcagaacctattaacgataatagtagctgccacttttttagcaaggtccctaatataggaactggatattacaaatactttatcgacttgggtcttctcatgagaatggaaaggaaactatctgataagatatcttctcaaaagatcaaggaaatggaagaaacagaagacttttaattcttatcaataacatatttttctatgatctgtcttttaaacgatggattttccacaaatgcgcctctcaagtccctcatagaatgatacacgtataaaaaatatagcataggcaatgactccttatttttagacattagatatgccaaaatcatagccccgcttctatttactcccgcagcacaatgaaccaacacgggctcgtttcgttgatcacatttagataaaaaggcggttacgtcgtcaaaatatttactaatatcggtagttgtatcatctaccaacggtatatgaataatattaatattagagttaggtaatgtatatttatccatcgtcaaatttaaaacatatttgaacttaacttcagatgatggtgcatccatagcatttttataatttcccaaatacacattattggttacccttgtcattatagtgggagatttggctctgtgcatatctccagttgaacgtagtagtaagtatttatacaaacttttcttatccatttataacgtacaaatggataaaactactttatcggtaaacgcgtgtaatttagaatacgttagagaaaaggctatagtaggcgtacaagcagccaaaacatcaacacttatattctttgttattatattggcaattagtgcgctattactctggtttcagacgtctgataatccagtctttaatgaattaacgagatatatgcgaattaaaaatacggttaacgattggaaatcattaacggatagcaaaacaaaattagaaagtgatagaggtagacttctagccgctggtaaggatgatatattcgaattcaaatgtgtggatttcggcgcctattttatagctatgcgattggataagaaaacatatctgccgcaagctattaggcgaggtactggagacgcgtggatggttaaaaaggcggcaaaggtcgatccatctgctcaacaattttgtcagtatttgataaaacacaagtctaataatgttattacttgtggtaatgagatgttaaatgaattaggttatagcggttattttatgtcaccgcattggtgttccgattttagtaatatggaatagtgttagataaatgcggtaacgaatgttcctgtaaggaaccataacagtttagatttaacgttaaagatgagcataaacataataaacaaaattacaatcaaacctataacattaatatcaaacaatccaaaaaatgaaatcagtggagtagtaaacgcgtacataactcctggataacgtttagtagctgccgttcctattctagaccaaaaattcggtttcatgttttcgaaacggtgttctgcaacaagtcggggatcgtgttctacatatttggcggcattatccagtatctgcctattgatcttcatttcgttttcaattctggctatttcaaaataaaatcccgatgatagacctccagactttataatttcatctacgatgttcagcgccgtagtaactctaataatataggctgataagctaacatcataccctcctgtatatgtgaatatggcatgatttttgtccattacaagctcggttttaactttattgcctgtaataatttctctcatctgtaggatatctatttttttgtcatgcattgccttcaagacgggacgaagaaacgtaatatcctcaataacgttatcgttttctacaataactacatattctacctttttattttctaactcggtaaaaaaattagaatcccatagggctaaatgtctagcgatatttcttttcgtttcctctgtacacatagtgttacaaaaccctgaaaagaagtgagtatacttgtcatcatttctaatgtttcctccagtccactgtataaacgcataatccttgtaatgatctggatcatccttgactaccacaacatttcttttttctggcataacttcgttgtcctttacatcatcgaacttctgatcattaatatgctcatgaacattaggaaatgtttctgatggaagtctatcaataactggcacaacaataacaggagttttcgccgccgccatttagttattgaaattaatcatatacaactctttaatacgagttatattttcgtctatccattgtttcacatttacatatttcgacaaaaagatataaaatgcgtattccaatgcttctctgtttaatgaattactaaaatatacaaacacgtcactgtctggcaataaatgatatcttagaatattgtaacaatttattttgtattgcacatgttcgtgatctatgagttcttcttcgaatggcataggatctccgaatctgaaaacgtataaataggagttagaataataatatttgagagtattggtaatatataaactctttagcggtataattagtttttttctctcaatttctatttttagatgtgatggaaaaatgactaattttgtagcattagtatcatgaactctaatcaaaatcttaatatcttcgtcacacgttagctctttgaagtttttaagagatgcatcagttggttcgaccgatggagtaggtgcaacaattttttgttcgatgtatgtatgtactggagccattgttttaactataatggtgcttgtatcgaaaaactttaatgcagatagcggaagctcttcgccgcgactttctacatcgtaattgggttctaacgccgatctctgaatggatactagttttctaagttctaatgtgattctctgaaaatgtaaatccaattcctccggcattatagatgtgtatacatcggtaaataaaactatagtatccaacgatcccttctcgcaaattctagtcttaaccaaaaaatcgtatataaccacggagatggcgtatttaagagtggattcttctaccgttttgttcttggatgtcatataggaaactataaagtccgcactactgttaagaatgattactaacgcaactatatagttcaaattaagcattttggaaacataaaataactctgtagacgatacttgactttcgaataagtttgcagacaaacgaagaaagaacagacctctcttaatttcagaagaaaactttttttcgtattcctgacgtctagagtttatatcaataagaaagttaagaattagtcggttaatgttgtatttcattacccaagtttgagatttcataatattatcaaaagacatgataatattaaagataaagcgctgactatgaacgaaatagctatatggttcgctcaaaaatatagtcttgttaaacgtggaaacgataactgtatttttaatcacgtcagcggcatctaaattaaatataggtatatttattccacacactctacaatatgccacaccatcttcataataaataaattcgttagcaaaattattaattttagtgaaatagttagcgtcaactttcatagcttccttcaatctaatttgatgctcacacggtgcgaattccactctaacatcccttttccatgcctcaggttcatcgatctctataatatctagttttttgcgtttcacaaacacaggctcgtctctcgcgatgagatctgtatagtaactatgtaaatgataactagatagaaagatgtagctatatagatgacgatcctttaagagaggtataataactttaccccaatcagatagactgttgttatggtcttcggaaaaagaatttttataaatttttccagtattttccaaatatacgtacttaacatctaaaaaatccttaatgataataggaatggataatccgtctattttataaagaaatacatatcgcacattatacttttttttggaaatgggaataccgatgtgtctacataaatatgcaaagtctaaatattttttagagaatcttaattggtccaaattcttttccaagtacggtaatagatttttcatattgaacggtatcttcttaatctctggttctagttccgcattaaatgatgaaactaagtcactatttttataactaacgattacatcacctctaacatcatcatttaccagaatactgatcttcttttgtcgtaaatacatgtctaatgtgttaaaaaaaagatcatacaagttatacgtcatttcatctgtggtattcttgtcattgaaggataaactcgtactaatctcttctttaacagcctgttcaaatttatatcctatatacgaaaaaatagcaaccagtgtttgatcatccgcgtcaatattctgttctatcgtagtgtataacaatcgtatatcttcttctgtgatagtcgatacgttataaaggttgataacgaaaatatttttatttcgtgaaataaagtcatcgtaggattttggacttatattcgcgtctagtagatatgcttttatttttggaatgatctcaattagaatagtctctttagagtccatttaaagttacaaacaactaggaaattggtttatgatgtataatttttttagtttttatagattctttattctatacttaaaaaatgaaaataaatacaaaggttcttgagggttgtgttaaattgaaagcgagaaataatcataaattatttcattatcgcgatatccgttaagtttgtatcgtaatggcgtggtcaattacaaataaagcggatactagtagcttcacaaagatggctgaaatcagagctcatctaaaaaatagcgctgaaaataaagataaaaacgaggatattttcccggaagatgtaataattccatctactaagcccaaaaccaaacgagccactactcctcgtaaaccagcggctactaaaagatcaaccaaaaaggaggaagtggaagaagaagtagttatagaggaatatcatcaaacaactgaaaaaaattctccatctcctggagtcagcgacattgtagaaagcgtggccgctgtagagctcgatgatagcgacggggatgatgaacctatggtacaagttgaagctggtaaagtaaatcatagtgctagaagcgatctctctgacctaaaggtggctaccgacaatatcgttaaagatcttaagaaaattattactagaatctctgcagtatcgacggttctagaggatgttcaagcagctggtatctctagacaatttacttctatgactaaagctattacaacactatctgatctagtcaccgagggaaaatctaaagttgttcgtaaaaaagttaaaacttgtaagaagtaaatgcgtgcacttttttataaagatggtaaactctttaccgataataattttttaaatcctgtatcagacgataatccagcgtatgaggttttgcaacatgttaaaattcctactcatttaacagatgtagtagtatatgaacaaacgtgggaggaggcgttaactagattaatttttgtgggaagtgattcaaaaggacgtagacaatacttttacggaaaaatgcatgtacagaatcgcaacgctaaaagagatcgtatttttgttagagtatataacgttatgaaacgaattaattgttttataaacaaaaatataaagaaatcgtccacagattccaattatcagttggcggtttttatgttaatggaaactatgttttttattagatttggtaaaatgaaatatcttaaggagaatgaaacagtagggttattaacactaaaaaataaacacatagaaataagtcccgatgaaatagttatcaagtttgtaggaaaggacaaagtttcacatgaatttgttgttcataagtctaatagactatataagccgctattgaaactgacggatgattctagtcccgaagaatttctgttcaacaaactaagtgaacgaaaggtatatgaatgtatcaaacagtttggtattagaatcaaggatctccgaacgtatggagtcaattatacgtttttatataatttttggacaaatgtaaagtccatatctcctcttccatcaccaaaaaagttaatagcgttaactatcaaacaaactgctgaagtggtaggtcatactccatcaatttcaaaaagagcttatatggcaacgactattttagaaatggtaaaggataaaaattttttagatgtagtatctaaaactacgttcgatgaattcctatctatagtcgtagatcacgttaaatcatctacggatggatgatatagatctttacacaaataattacaaaaccgataaatggaaatggataagcgtatgaaatctctcgcaatgaccgctttctttggggagctaagcacattagatattatggcattgataatgtctatatttaaacgccatccaaacaataccattttttcagtggataaggatggtcagtttatgattgatttcgaatacgataattataaggcttctcaatatttggatctgaccctcactccgatatctggagatgaatgcaagactcacgcatcgagtatagccgaacaattggcgtgtgtggatattattaaagaggatattagcgaatatatcaaaactactccccgtcttaaacgatttataaaaaaataccgcaatagatcagatactcgcatcagtcgagatacagaaaagcttaaaatagctctagctaaaggcatagattacgaatatataaaagacgcttgttaataagtaaatgaaaaaaaactagtcgtttataataaaacacgatatggatgccaacgtagtatcatcttctactattgcgacgtatatagacgctttagcgaagaatgcttcggaattagaacagaggtctaccgcatacgaaataaataatgaattggaactagtatttattaagccgccattgattactttgacaaatgtagtgaatatctctacgattcaggaatcgtttattcgatttaccgttactaataaggaaggtgttaaaattagaactaagattccattatctaaggtacatggtctagatgtaaaaaatgtacagttagtagatgctatagataacatagtttgggaaaagaaatcattagtgacggaaaatcgtcttcacaaagaatgcttgttgagactatcgacagaggaacgtcatatatttttggattacaagaaatatggatcctctatccgactagaattagtcaatcttattcaagcaaaaacaaaaaactttacgatagactttaagctaaaatattttctaggatccggtgcccagtctaaaagttctttattacacgctattaatcatccaaagtcaaggcctaatacatctctggaaatagaatttacacctagagacaatgaaacagttccatatgatgaactaataaaggaattgacgactctctcgcgtcatatatttatggcttctccagagaatgtaattctttctccgcctattaacgcgcctataaaaacctttatgttgcctaaacaagatatagtaggtttggatctggaaaatctatatgccgtaactaagactgacggcattcctataactatcagagttacatcaaacgggttgtattgttattttacacatcttggttatattattagatatcctgttaagagaataatagattccgaagtagtagtctttggtgaggcagttaaggataagaactggaccgtatatctcattaagctaatagagcctgtgaatgcaatcaatgatagactagaagaaagtaagtatgttgaatctaaactagtggatatttgtgatcggatagtattcaagtcaaagaaatacgaaggtccgtttactacaactagtgaagtcgtcgatatgttatctacatatttaccaaagcaaccagaaggtgttattctgttctattcaaagggacctaaatctaacattgattttaaaattaaaaaggaaaatactatagaccaaactgcaaatgtagtatttaggtacatgtccagtgaaccaattatctttggagagtcgtctatctttgtagagtataagaaatttagcaacgataaaggctttcctaaagaatatggttctggtaagattgtgttatataacggcgttaattatctaaataatatctattgtttggaatatattaatacacataatgaagtgggtattaagtccgtggttgtacctattaagtttatagcagaattcttagttaatggagaaatacttaaacctagaattgataaaaccatgaaatatattaactcagaagattattatggaaatcaacataatatcatagtcgaacatttaagagatcaaagcatcaaaataggagatatctttaacgaggataaactatcggatgtgggacatcaatacgccaataatgataaatttagattaaatccagaagttagttattttacgaataaacgaactagaggaccgttgggaattttatcaaactacgtcaagactcttcttatttctatgtattgttccaaaacatttttagacgattccaacaaacgaaaggtattggcgattgattttggaaacggtgctgacctggaaaaatacttttatggagagattgcgttattggtagcgacggatccggatgctgatgctatagctagaggaaatgaaagatacaacaaattaaactctggaattaaaaccaagtactacaaatttgactacattcaggaaactattcgatccgatacatttgtctctagtgtcagagaagtattctattttggaaagtttaatatcatcgactggcagtttgctatccattattcttttcatccgagacattatgctaccgtcatgaataacttatccgaactaactgcttctggaggcaaggtattaatcactaccatggacggagacaaattatcaaaattaacagataaaaagacttttataattcataagaatttacctagtagcgaaaactatatgtctgtagaaaaaatagctgatgatagaatagtggtatataatccatcaacaatgtctactccaatgactgaatacattatcaaaaagaacgatatagtcagagtgtttaacgaatacggatttgttcttgtagataacgttgatttcgctacaattatagaacgaagtaaaaagtttattaatggcgcatctacaatggaagatagaccatctacaagaaactttttcgaactaaatagaggagccattaaatgtgaaggtttagatgtcgaagacttacttagttactatgttgtttatgtcttttctaagcggtaaataataatatggtatgggttctgatctcccagttctaaatgcattaaataattccaatagagcgatttttgttcctataggaccttccaactgtggatactctgtattgttaatagatatattaatacttttgtcgggtaacagaggttctacgtcttttaaaaataaaagtttgataacatctggcctgttcataaataaaaacttggcgattctatatatactcttattatcaaatctagccattgtcttatagatgtgagctactgtaggtgtaccatttgattttctttctaatactatatatttctctcgaagaagttcttgcacatcatctgggaataaaatactactgttgagtaaatcagttattttttttatatcgatattgatggacatttttatagttaaggataataagtatcccaaagtagataacgacgataacgaagtatttatacttttaggaaatcacaatgactttatcagattaaaattaacaaaattaaaggagcatgtatttttttctgaatatattgtgactccagatacatatggatctttatgcgtcgaattaaatgggtctagttttcagcacggcggtagatatatagaggtggaggaatttatagatgctggaagacaagttagatggtgttctacatccaatcatatatctaaagatatacccgaagatatgcacactgataaatttgtcatttatgatatatacacttttgacgctttcaagaataaacgattggtattcgtacaggtacctccgtcgttaggagatgatagtcatttgactaatccgttattgtctccgtattatcgtaattcagtagccagacaaatggtcaatgatatgatttttaatcaagattcatttttaaaatatttattagaacatctgattagaagccactatagagtttctaaacatataacaatagttagatacaaggataccgaagaattaaatctaacgagaatatgttataatagagataagtttaaggcgtttgtattcgcttggtttaacggcgtttcggaaaatgaaaaggtactagatacgtataaaaaggtatctaatttgatataatgaattcagtgactgtatcacacgcgccatatactattacttatcacgatgattgggaaccagtaatgagtcaattggtagagttttataacgaagtagccagttggctgctacgagacgagacgtcgcctattcctgataagttctttatacagttgaaacaaccgcttagaaataaacgagtatgtgtgtgcggtatagatccgtatccgaaagatggaactggtgtaccgttcgaatcaccaaattttacaaaaaaatcaattaaggagatagcttcatctatatctagattaaccggagtaattgattataaaggttataaccttaatataatagacggggttataccctggaattattacttaagttgtaaattaggagaaacaaaaagtcacgcgatctactgggataagatttccaagttactgctgcagcatataactaaacacgttagtgttctttattgtttgggtaaaacagatttctcgaatatacgggccaagttagaatccccggtaactaccatagtcggatatcatccagcggctagagaccgccaattcgagaaagatagatcatttgaaattatcaacgttttactggaattagacaacaaggcacctataaattgggctcaagggtttatttattaatgctttagtgaaattttaacttgtgttctaaatggatgcggctattagaggtaatgatgttatctttgttcttaagactataggtgtcccgtcagcgtgcagacaaaatgaagatccaagatttgtagaagcatttaaatgcgacgagttagaaagatatattgagaataatccagaatgtacactattcgaaagtcttagggatgaggaagcatactctatagtcagaattttcatggatgtagatttagacgcgtgtctagacgaaatagattatttaacggctattcaagattttattatcgaggtgtcaaactgtgtagctagattcgcgtttacagaatgcggcgccattcatgaaaatgtaataaaatccatgagatctaatttttcattgactaagtctacaaatagagataaaacaagttttcatattatctttttagacacgtataccactatggatacattgatagctatgaaacgaacactattagaattaagtagatcatctgaaaatccactaacaagatcgatagacactgccgtatataggagaaaaacaactcttcgggttgtaggtactaggaaaaatccaaattgcgacactattcatgtaatgcaaccaccgcatgataatatagaagattacctattcacttacgtggatatgaacaacaatagttattacttttctctacaacaacgattggaggatttagttcctgataagttatgggaaccagggtttatttcattcgaagacgctataaaaagagtttcaaaaatattcattaattctataataaactttaatgatctcgatgaaaataattttacaacggtaccactggtcatagattacgtaacaccttgtgcattatgtaaaaaacgatcgcataaacatccgcatcaactatcgttggaaaatggtgctattagaatttacaaaactggtaatccacatagttgtaaagttaaaattgttccgttggatggtaataaactgtttaatattgcacaaagaattttagacactaactctgttttattaaccgaacgaggagaccatatagtttggattaataattcatggaaatttaacagcgaagaacccttgataacaaaactaattttgtcaataagacatcaactacctaaggaatattcaagcgaattactctgtccaagaaaacgaaagactgtagaagctaacatacgagacatgttagtagattcagtagagaccgatacctatccggataaacttccgtttaaaaatggtgtattggacctggtagacggaatgttttactctggagatgatgctaaaaaatatacgtgtactgtatcaaccggatttaaatttgacgatacaaagttcgtcgaagacagtccagaaatggaagagttaatgaatatcattaacgatatccaaccattaacggatgaaaataagaaaaatagagagctatatgaaaaaacattatctagttgtttatgcggtgctaccaaaggatgtttaacattcttttttggagaaactgcaactggaaagtcgacaaccaaacgtttgttaaagtctgctatcggtgacctgtttgttgagacgggtcaaacaattttaacagatgtattggataaaggacctaatccatttatcgctaacatgcatttgaaaagatctgtattctgtagcgaactacctgattttgcctgtagtggatcaaagaaaattagatctgacaatattaaaaagttgacagaaccttgtgtcattggaagaccgtgtttctccaataaaattaataatagaaaccatgcgacaatcattatcgatactaattacaaacctgtttttgataggatagataacgcattaatgagaagaattgccgtcgtgcgattcagaacacacttttctcaaccttctggtagagaggctgctgaaaataatgacgcgtacgataaagtcaaactattagacgaggggttagatggtaaaatacaaaataatagatatagattcgcatttctatacttgttggtgaaatggtacagaaaatatcatgttcctattatgaaactatatcctacacccgaagagattcctgactttgcattctatctcaaaataggtactctgttagtatctagctctgtaaagcatattccattaatgacggacctctccaaaaagggatatatattgtacgataatgtggtcactcttccgttgactactttccaacagaaaatatccaagtattttaattctagactatttggacacgatatagagagcttcatcaatagacataagaaatttgccaatgttagtgatgaatatctgcaatatatattcatagaggatatttcatctccgtaaatatatgctcatatatttatagaagatatcacatatctaaatgaataccggaatcatagatttatttgataatcatgttgatagtataccaactatattacctcatcagttagctactctagattatctagttagaactatcatagatgagaacagaagcgtgttattgttccatattatgggatcaggtaaaacaataatcgctttgttgttcgccttggtagcttccagatttaaaaaggtttacattctagtgcctaatatcaacattttgaaaatttttaattataatatgggtgtagctatgaacttgtttaatgacgaattcatagctgagaatatctttattcattccacaacaagtttttattctcttaattataacgataacgtcattaattataacggattatctcgctacaataactctatttttatcgttgatgaggcacataatatctttgggaataatactggagaacttatgaccgtgataaaaaataaaaacaagattccttttctactattgtctggatctcccattactaacacacctaatactctgggtcatattatagatttaatgtccgaagagacgatagattttggtgagattattagtcgtggtaagaaagtaattcagacacttcttaacgaacgcggtgtgaatgtacttaaggatttgcttaaaggaagaatatcatattacgaaatgcctgataaagatctaccaacgataagatatcacggacgtaagtttctagatactagagtagtatattgtcacatgtctaaacttcaagagagagattatatgattactagacgacagctatgttatcatgaaatgtttgataaaaatatgtataacgtgtcaatggcagtattgggacaacttaatctgatgaataatttagatactttatttcaggaacaggataaggaattgtacccaaatctgaaaataaataatggcgtgttatacggagaagaattggtaacgttaaacattagttccaaatttaaatactttattaatcggatacagacactcaacggaaaacattttatatacttttctaattctacatatggcggattggtaattaaatatatcatgctcagtaatggatattctgaatataatggttctcagggaactaatccacatatgataaacggcaaaccaaaaacatttgctatcgttactagtaaaatgaaatcgtctttagaggatctattagatgtgtataattctcctgaaaacgatgatggtagtcaattgatgtttttgttttcgtcaaacattatgtccgaatcctatactctgaaagaggtaaggcatatttggtttatgactatcccagatactttttctcaatacaaccaaattcttggacgatctattagaaaattctcttacgccgatatttctgaaccagttaatgtatatcttttagccgccgtatattccgatttcaatgacgaagtaacgtcattaaacgattacacacaggatgaattgattaatgttttaccatttgacatcaaaaagctgttatatctaaaatttaagactaaagaaacgaatagaatatactctattcttcaagagatgtctgaaacgtattctcttccaccacatccatcaattgtaaaagttttattgggagaattggtcagacaatttttttataataattctcgtattaagtataacgactccaagttacttaaaatggttacatcagttataaaaaataaagaagacgctaggaattacatagatgatattgtaaacggtcacttctttgtatcgaataaagtatttgataaatctcttttatacaaatacgaaaacgatattattacagtaccgtttagactttcctacgaaccatttgtttggggagttaactttcgtaaagaatataacgtggtatcttctccataaaactgatgaaatatataaagaaataaatgtcgagctttgttaccaatggataccttccagttacattggaaccacacgagctgacgttagacataaaaactaatattaggaatgccgtatataagacgtatctccatagagaaattagtggtaaaatggccaagaaaatagaaattcgtgaagacgtggaattacctctcggcgaaatagttaataattctgtagttataaacgttccgtgtgtaataacctacgcgtattatcacgttggggatatagtcagaggaacattaaacatcgaagatgaatcaaatgtaactattcaatgtggagatttaatctgtaaactaagtagagattcgggtactgtatcatttagcgattcaaagtactgcttttttcgaaatggtaatgcgtatgacaatggcagcgaagtcactgccgttctaatggaggctcaacaaggtatcgaatctagttttgtttttctcgcgaatatcgtcgactcataaaaaagagaatagcggtaagtataaacacgaatactatggcaataattgcgaatgttttattctcttcgatatatttttgataatatgaaaaacatgtctctctcaaatcggacaaccatctcataaaatagttctcgcgcgctggagaggtagttgctgctcgtataatctccccagaataatatacttgcgtgtcgtcgttcaatttatacggatttctatagttctctgttatataatgcggttttccatcatgattagacgacgacaatagtgttctgaatttagatagttgatcagaatgaatgtttattggcgttggaaaaattatccatacagcgtctgcagagtggttgatagttgttcctagatatgtaaaataatccaacttactaggcagcaaattgtctagataaaatactgaatcaaacggtgcagacgtattggcggatctaatggaatccaattgattaactatcttttgaaaatatacatttttatgatccaatacttgtaagaatatagaaataatgataagtccatcatcgtgtttttttgcctcttcataagaactatattttttcttattccaatgaacaagattaatctctccagagtatttgtacacatctatcaagtgattggatccataatcgtcttcctttccccaatatatatgtagtgatgataacacatattcattggggagaaaccctccacttatatatcctcctttaaaattaatccttactagttttccagtgttctggatagtggttggtttcgactcattataatgtatgtctaacggcttcaatcgcgcgttagaaattgcttttttagtttctatattaataggagatagttgttgcggcatagtaaaaatgaaatgataactgtttaaaaatagctcttagtatgggaattacaatggatgaggaagtgatatttgaaactcctagagaattaatatctattaaacgaataaaagatattccaagatcaaaagacacgcacgtgtttgctgcgtgtataacaagtgacggatatccgttaataggagctagaagaacttcattcgcgttccaggcgatattatctcaacaaaattcagattctatctttagagtatccactaaactattacggtttatgtactacaatgaactaagagaaatctttagacggttgagaaaaggttctatcaacaatatcgatcctcactttgaagagttaatattattgggtggtaaactagataaaaaggaatctattaaagattgtttaagaagagaattaaaagaggaaagtgatgaacgtataacagtaaaagaattcggaaatgtaattctaaaacttacaacgcgcgataaattatttaataaagtatatataggttattgcatggcgtgttttattaatcaatcgttggaggatttatcgcatactagtatttacaatgtagaaattagaaagattaaatcattaaatgattgtattaacgacgataaatacgaatatctgtcttatatttataatatgctagttaatagtaaatgaacttttacagatctagtataattagtcagattattaagtataatagacgactagctaagtctattatttgcgaggatgactctcaaattattacactcacggcattcgttaaccaatgcctatggtgtcataaacgagtatccgtgtccgctattttattaactactgataacaaaatattagtatgtaacagacgagatagttttctctattctgaaataattagaactagaaacatgtttagaaagaaacgattatttctgaattattccaattatttgaacaaacaggaaagaagtatactatcgtcatttttttctctagatccagctactgctgataatgatagaatagacgctatttatccgggtggcatacccaaaaggggtgagaatgttccagagtgtttatccagggaaattaaagaagaagttaatatagacaattcttttgtattcatagacactcggttttttattcatggcatcatagaagataccattattaataaattttttgaggtaatcttctttgtcggaagaatatctctaacgagtgatcaaatcattgatacatttaaaagtaatcatgaaatcaaggatctaatatttttagatccgaattcaggtaatggactccaatacgaaattgcaaaatatgctctagatactgcaaaactcaaatgttatggccatagaggatgttattacgaatcattaaaaaaattaactgaggatgattgattagaaaatataaattaatttaccatcgtgtatttttataacgggattgtccggcatatcatgtagatagttaccgtctacatcgtatactcgaccatctacgcctttaaatcctctatttattgacattaatctattagaattggaataccaaatattagtaccctcaattagtttattggtaatatttttgttagacgatagatcgatggctcttgaaaccaaggttttccaaccggactcattgtcgatcggtgagaagtctttttcattagcatgaatccattctaatgatgtatgtttaaacactctaaacaattggacaaattcttttgatttgctttgaatgatttcaaataggtcttcgtctacagtaggcataccattagataatctagccattataaagtgcacgtttacatatctacgttctggaggagtaagaacgtgactattgagacgaatggctcttcctactatctgacgaagagacgcctcgttccatgtcatatctaaaatgaagatatcattgattgagaagaagctaataccctcgcctccactagaagagaatacgcatgttttaatgcattctccgttagtgtttgattcttggttaaactcagccaccgccttgattctagtatcttttgttctagatgagaactctatattagagataccaaagactttgaaatatagtaataagatttctattcctgactgattaacaaatggttcaaagactagacatttaccatgggatgctaatattcccaaacatacatctataaatttgacgcttttctcttttaattcagtaaatagagagatatcagccgcactagcatcccctcccaatagttctccccttttaaaggtatctaatgcagatttagaaaattctctatctcttaatgaatttttaaaatcattatatagtgttgctatctcttgcgcgtattcgcccggatcacgattttgtctttcaggaaagctatcgaacgtaaacgtagtagccatacgtctcagaattctaaatgatgatatacctgtttttatttcagcgagtttagccttttgataaatttcttcttgctttttcgacatattaacgtatcgcattaatactgttttcttagcgaatgatgcagacccttctacgtcatcaaaaatagaaaactcgttattaactatgtacgaacataggcctcctagtttggagactaattctttttcatcaactagacgtttattctcaaatagcgattggtgttgtaaggatcctggtcgtagtaagttaaccaacatggtgaattcttgcacactattgacgataggtgtagccgataaacaaatcatcttatggttttttaatgcgatggtcttagataaaaaattatatactgaacgagtaggacggatcttaccatcttctttgattaatgatttagaaatgaagttatgacattcatcaataatgacgcatattctactcttggaattaatagttttgatattagtaaaaaatttatttctaaaattttgatcatcgtaattaataaaaatacaatccttcgttatctctggagcgtatctgagtatagtgttcatccaaggatcttctatcaaagcctttttcaccaataagataatagcccaattcgtataaatatccttaagatgtttgagaatatatacagtagtcattgttttaccgacacccgtttcatggaacaataaaagagaatgcatactgtctaatcctaagaaaactcttgctacaaaatgttgataatccttgaggcgtactacgtccgaccccatcatttcaacaggcatattagtagttctgcgcaatgcataatcgatataggccgcgtgtgatttactcatttatgagtgataagtaataactatgttttaaaaatcacagcagtagtttaactagtcttctctgatgtttgttttcgatactttttgaatcagaagtcatactagaataaagcaacgagtgaacgtaatagagagcttcgtatactctattcgaaaactctaagaacttattaatgaattccgtatccactggattgtttaaaatactaaattgaacactgttcacatccttccaagaagaagacttagtgacggacttaacatgagacataaataaatccaaattttttttacaaacatcactagccaccataatggcgctatcttttaaccagctatcgcttacgcattttagcagtctaacatttttaaagagactacaatatattctcatagtatcgattacacctctaccgaataaagttggaagtttaataatacaatatttttcgtttacaaaatcaaataatggtcgaaacacgtcgaaggttaacatcttataatcgctaatgtatagattgttttcagtgagatgattattagatttaatagcatctcgttcacgtttgaacagtttattgcgtgcgctgaggtcggcaactacggcgtccgctttagtactcctcccataatactttacgctattaatctttaaaatttcatagactttatctagatcgctttctggtaacatgatatcatgtgtaaaaagttttaacatgtcggtcggcattctatttagatcattaactctagaaatctgaagaaagtaattagctccgtattccagactaggtaatgggcttttacctagagacagattaagttctggcaatgtttcataaaatggaagaaggacatgcgttccctcccggatattttttacaatttcatccatttacaactctatagtttgttttcattattattagttattatctcccataatcttggtaatacttaccccttgatcgtaagataccttatacaggtcattacatacaactaccaattgtttttgtacataatagattggatggttgacatccatggtggaataaactactcgaacagatagtttatctttccccctagatacattagccgtaatagttgtcggcctaaagaatatctttggtgtaaagttaaaagttagggttcttgttccattattgctttttgtcagtagttcattataaattctcgagatgggtccgttctctgaatatagaacatcatttccaaatctaacttctagtctagaaataatatcggtcttattcttaaaatctattcccttgatgaagggatcgttaatgaacaaatccttggcctttgattcggctgatctattatctccgttatagacgttacgttgactagtccaaagacttacaggaatagatgtatcgatgatgttgatactatgtgatatgtgagcaaagattgttctcttagtggcatcactatatgttccagtaatggcggaaaactttttagaaatgttatatataaaagaattttttcgtgttccaaacattagcagattagtatgaagataaacactcatattatcaggaacattatcaatttttacatacacatcagcatcttgaatagaaacgataccatcttctggaacctctacgatctcggcagactccggataaccagtcggtggaccatcgctaacaataactagatcatccaacaatctactcacatatgcatctatataatctttttcatcttgtgagtaccctggatacgaaataaatttattatccgtatttccataataaggtttagtataaacagagagcgatgttgccgcatgaacttcagttacagtcgccgttggttggtttatttgacctattactctcctaggtttctctataaatgatggtttaatttgtacattcttaaccatatatccaataaagctcaattcaggaacataaacaaattctttgttgaacgtttcaaagtcgaacgaagagtcacgaataacgatatcggatactggattgaaggttaccgttacggtaatttttgaatcggatagtttaagactgctgaatgtatcttccacatcaaacggagttttaatataaacgtatactgtagatggttctttaatagtgtcattaggagttaggccaatagaaatatcattaagttcactagaatatccagagtgtttcaaagcaattgtattattgatacaattattatataattcttcgccctcaatttcccaaataacaccgttacacgaagagatagatacgtgattaatacatttatatccaacatatggtacgtaaccgaatcttcccatacctttaacttctggaagttccaaactcagaaccaaatgattaagcgcagtaatatactgatccctaatttcgaagctagcgatagcctgattgtctggaccatcgtttgtcataactccggatagagaaatatattgcggcatatataaagttggaatttgactatcgactgcgaagacattagaccgtttaatagagtcatccccaccgatcaaagaattaatgatagtattattcattttctatttaaaatggaaaaagcttacaataaactccgtagagaaatatctataatttgtgagttttccttaaagtaacagcttccgtaaacgccgtctttatctcttagtaagtttattgtatttataaccttttccttatcttcatagaatactaaaggcaacaaagaaatttttggttcttctctaagagctacgtgagacttaaccatagacgccaacgaatccctacatattttagaacagaaatacccaacttcaccacccttgaatgtctcaatactaataggtttaaaaaccaaatcttgattacaaaaccaacacttatcaattacactatttgtcttaatagacacatctgccatagatttataatactttggtagtatacaagcgagtgcttcttctttagcgggcttaaagactgctttaggtgctgaaataaccacatctggaaggcttactcgcttagccatttaattacggaactatttttttatacttctaatgagcaagtagaaaacctctcatctacaaaaacatactcgtgtccataatcctctaccatagttacacgttttttagatctcatatgtgctaaaaagttttcccatactaattggttactattatttttcgtataatttttaacagtttgaggttttagatttttagttacagaagtgatatcgaatattttatccaaaaagaatgaataattaattgtcttagaaggagtgttttcttggcaaaagaataccaagtgcttaaatatttctactacttcattaatcttttctgtactcagattcagtttctcatcttttacttgattgattatttcaaagactaacttataatcctttttatttattctctcgttagccttaagaaaactagatacaaaatttgcatctacatcatccgtggatatttgatttttttccatgatatccaagagttccgagataatttctccagaacattgatgagacaataatctccgcaatacatttctcaaatgaataagtttattagacacatggaagtttgactttttttgtacctttgtacatttttgaaataccgactcgcaaaaaatacaatattcatatccttgttcagatactataccgttgtgtctacaaccgctacataatcgtagattcatgttaacactctacgtatctcgtcgtccaatattttatataaaaacattttatttctagacgttgccagaaaatcctgtaatatttttagttttttgggctgtgaataaagtatcgccctaatattgttaccgtcttccgccaatatagtagttaaattatccgcacatgcaaaagaacaccgcttaggcggattcagtacaatgttatatttttcgtaccaactcatttaaatatcataatctaaaatagttctgtaatatgtctagcgctaatatattgatcataatcctgtgcataaattaagatacaacaatgtctcgaaatcatcgacatggcttcttccatagttagaagatcgtcgtcaaagttagcaacgtgattcatcaacatttgctgttttgaggcagcaaatactgaaccgtcgccattcaaccattcataaaaaccatcgtctgaatccattgataatttcttgtactggtttttgagagctcgcatcaatctagcatttctagctcccggattgaaaacagaaagaggatcgtacatccagggtccattttctgtaaatagaatcgtataatgtcccttcaagaagatatcagacgatccacaatcaaagaattggtctccgagtttgtaacaaactgcggactttaacctatacatgataccgtttagcatgatttctggtgatacgtcaatcggagtatcatctattagagatctaaagccggtgtaacattctccaccaaacatattcttattctgacgtcgttctacataaaacatcattgctccattaacgataacaggggaatgaacagcactacccatcacattagttcccaatggatcaatgtgtgtaactccagaacatcttccatatcctatgttaggaggagcgaacaccactcttccactattgccatcgaatgccatagaataaatatccttggaattgatagaaatcggactgtcggatgttgtgatcatcttcataggattaacaactatgtatggtgccgcctgaagtttcatatcgtaactgatgccgtttataggtctagccacagaaaccaacgtaggtctaaatccaactatagacaaaatagaagccaatatctgttcctcatctgtcataacttgagagcatccagtatgaataatcttcattagatggggatctaccgcatcatcatcgttacaataaaaaattcccattctaatgttcataattgcttttctaatcatggtatgcatgtttgctctctgaatctctgtggaaattagatctgatacacctgtaatcactatcggattatcctccgtaagacgattaaccaacaacatataattataagactttacttttctaaattcataaagttgctggattaggctataggtgtctccatgtacatacgcgttctcgagcgcaggaagtttaataccgaatagtgccatcagaataggatgaatatagtaattagtttctggttttctataaataaaagacaaatcttgtgaactagacatatcggtaaaatgcatggattggaatcgtgtagtcgacagaagaatatgatgattagatggagagtatattttatctaactctttgagttggtcaccgattctaggactagctcgagaatgaataagtactaaaggatgagtacatttcacagaaacactagcattgttcaatgtgctctttacatgggtaaggagttgaaatagctcgtttctatttgttctgacaatatttagtttattcataatgttaagcatatcctgaatagtaaagttagatgtgtcatacttgttagtagttagatatttagcaattgcattcccatcatttctcaatctcgtactccaatcatgtgtagatgctacttcgtcgatggaaaccatacaatcctttttgataggctgttgagattgattatttcctgcacgtttaggtttggtacgttgatttctagcccctgcggatataaagtcatcgtctacaatttgggacaatgaattgcatacactacaagacaaagatttatcagaagtgtgaatatgatcttcatctaccaaagaaagagtttgattagtataactagattttagtcctgcgttagatgttaaaaaaacatcgctattgaccacggcttccattatttatattcgtagtttttactcgaaagcgtgattttaatatccaatcttattacttttggaatcgttcaaaacctttgactagttgtagaatttgatctattgccctacgcgtatactcccttgcatcatatacgttcgtcaccagatcgtttgtttcggcctgaagttggtgcatatctctttcaacattcgacatgagatccttaagggccatatcgtctagattttgttgagatgctgctcctggatttggattttgttgtgctgttgtacatactgtaccaccagtaggtgtaggagtacatacagtggccacaataggaggttgaggaggtgtaaccgttggagtagtacaagaaatatttccatccgattgttgtgtacatgtagttgttggtaacgtctgagaaggttgggtagatggcggtgtcgtcgtcttttgatctttattaaatttagagataatatcctgaacagcattgctcggcgtcaacgctggaaggagtgaactcgccggcgcatcagtatcttcagacagccaatcaaaaagattagacatatcagatgatgtattagtttgttgtcgtggttttggtgtaggaacagtactactaggtagaagaataggagccggtgtagctgttggaaccggctgtggagttatatgaatagttggttgtagcggttggataggctgtctgctggcgaccgtcatattatctctagctagttgttctcgcaactgtctttgataatacgactcttgagactttagtcctatttcaatcgcttcatcctttttcgtatccggatccttttcttcagaataatagattgacgactttggtgtagaggattctgccagcccctgtgagaacttgttaaagaagtccatttaaggctttaaaattgaattgcgattataagattaaatggcagacacagacgatattatcgactatgaatccgatgatctcaccgaatacgaggatgatgaagaagaggaagaagatggagagtcactagaaactagtgatatagatcccaaatcttcttataagattgtagaatcagcatccactcatatagaagatgcgcattccaatcttaaacatatagggaatcatatatctgctcttaaacgacgctatactagacgtataagtctatttgaaatagcgggtataatagcagaaagctataacttgcttcaacgaggaagattacctctagtttcagaattttctgacgaaacgatgaagcaaaatatgctacatgtaattatacaagagatagaggagggttcttgtcctatagtcatcgaaaagaacggagaattgttgtcggtaaacgattttgacaaagatggtctaaaattccatctagactatattatcaaaatttggaaacttcaaaaacgatattagaatttatacgaatatcgttctctaaatgtcacaatcaagtctcgcatgttcagcaatttattgtcgtactttatatcgtgttcattaacgatatcttgcaaaatagtaatgattctatcttccttcgatagatattcttcagagattattgtcttatattttttcttgttatccgatatgaatttgataagactttgaacattattgatacccgtctgtttaattttttctacagatattttagttttggcagattctatcgtatctgtcaatagacatccaacatcgacattcgacgtcaattgtctataaatcaacgtataaattttagaaataacattagcgaattgttgtgcattgatgtcgttattctgaaacagtatgattttaggtagcattttcttaacaaagagaacgtatttattgttactcagttgaacagatgatatatccagattactaacgcatctgattccgtataccaaactttcagaagaaatggtgtacaattgtttgtattcattcaatgtctctttttcagaaattagtttagagtcgaatactgcaataattttcaagagatagttttcatcagataagattttatttagtgtagatatgataaaactattgttttgttggagaacttgatacgccgcgttctctgtagtcgacgctctcaaatgggaaacaatctccattatttttttggaatcggatactatatcttcggtatcttgacgcagtctagtatacatagagttaagagagattagagtttgtacattaagcaacatgtctctaaatgtggctacaaacttttcctttttcacataatctagtttattatataccgatttcacaacggcaccagatttaaggaaccagaatgaaaaactctgataactacaatatttcatcatagttacgattttatcatcttctatagttggtgtaatagcgcatacctttttctccaagactggaaccaacgtcataaaaatgtttaaatcaaaatccatatcaacatctgatgcgctaagaccagtctcgcgttcaagattatctttactaatggtgacgaactcatcatatagaactctaagtttgtccattatttatttacagatttagttgtttaatttatttgtgctcttccagagttgggatagtatttttctaacgtcggtattatattattaggatctacgttcatatgtatcataatattaatcatccacgttttgataaatctatctttagcttctgaaataacgtatttaaacaaaggagaaaaatatttagctacggcatcagacgcaataacattttttgtaaatgtaacgtatttagacgacagatcttcgttaaaaagttttccatctatgtagaatccatcggttgttaacaccattcccgcgtcagattgaataggagtttgaatagtttgttttggaaatagatccttcaataacttatagttgggtgggaaaaaatcgattttatcactagactctttcttttttactatcattacctcatgaactatttcttgaatgagtatatgtattttctttcctatatcggacgcgttcattggaaaatataccatgtcgttaactataagaatatttttatcctcgtttacaaactgaataatatcagatgtagttcgtaaacgaactatatcatcaccagcacaacatctaactatatgatatccactagtttcctttagccgtttattatcttgttccatattagcagtcattccatcatttaagaaggcgtcaaagataatagggagaaatgacattttggattctgttacgactttaccaaaattaaggatatacggacttactatctttttctcaacgtcgatttgatgaacacacgatgaaaatgtgcttctatgagattgatcatgtagaaaacaacaagggatacaatatttccgcatatcatgaaatatattaagaaatcccaccttattatatttccccaaaggatccatgcatgtaaacattatgccgttatcattaataaagacttctttctcatcggatttgtaaaagttgttactgatttttttcattccaggatctagataattaataatgatgggttttctattcttattctttgtattttggcatatcctagaccagtaaacagtttccactttggtaaaatcagcagacttttgaacgctattaaacatggcattaatggcaataactaaaaatgtaaaatatttttctatgttaggaatatggtttttcactttaatagatatatggtttttggccaaaatgatagatatttttttatccgaggatagtaaaatattattagtcgccgtctctataaaaatgaagctagtctcgatatccaattttattctagaattgataggagtcgccaaatgtaccttatacgttatatctcccttgatgcgttccatttgtgtatctatatcggacacaagatctgtaaatagttttacgttattaatcatcacggtatcgccgtcgctagataacgctaatgtaccatccaagtcccaaatggagagatttaactgttcatcgtttagaataaaatgattaccggtcatattaataaagtgttcatcgtatctagataacaacgacttataattaatgtccaagtcttgaactcgctgaatgatcttttttaacccagttagttttagattggtacgaaatatattgttaaactttgattctacagtaatgtccaaatctagttgtggaaatacttccatcaacattgtttcaaacttgataatattattatctacatcttcatacgatccaaattccggaatagatgtatcacatgctcttgccacccagataaccaaaaagtcacacgctccaggatatacattgtataaaaagctatcgttttttagtagtgtttttttctgagtatatacgaagggattaaaaatagtattatcaacgtaactatattccaaattattcttatgagaatagataataatatcgtccttaatatctaacaaatttcctaaatatccctttaattgagtcattcgaagcgtcaatagaatatgtctcttaactatttccggctgttgtatatttaaatgacttcgtaaaaaataatatatgggcgacttctcatctatgtaatcatatggagtgagatatagggctcgttctacctcctgccccttacccacctgtaataccaattgcggacttactatatatcgcatatttatatcgtggggtaaagtgaaaatctactaccgatgatgtaagtcttacaatgttcgaaccagtaccagatcttaatttggaggcctccgtagaactaggggaggtaaatatagatcaaacaacacctatgataaaggaaaatagcggttttatatcccgtagtagacgtctattcgcccatagatctaaggatgatgagagaaaactagcactacgattctttttacaaagactttattttttagatcatagagagattcattatttgttcagatgcgttgacgctgtaaaagacgtcactattaccaaaaaaaataacattatcgtggcgccttatatagcacttttaactatcgcatcaaaaggatgcaaacttacagaaacaatgattgaagcattttttccagaactatataatgaacatagtaagaaatttaaattcaactctcaagtatccatcatccaagaaaaactcggataccagtttggaaactatcacgtttatgattttgaaccgtattactctacagtagctctggctattcgagatgaacattcatctggcatttttaatatccgtcaagagagttatctggtaagttcattatctgaaataacatatagattttatctaattaatctaaaatctgatcttgttcaatggagtgctagtacgggcgctgtaattaatcaaatggtaaatactgtattgattacagtgtatgaaaagttacaactggtcatagaaaatgattcacaatttacatgttcattggctgtggaatcaaaacttccaataaaattacttaaagatagaaatgaattatttacaaaattcattaacgagttaaaaaagaccagttcattcaagataagcaaacgcgataaggatacgctactaaaatattttacttaggactggagttagaatttatagacgactcatttcgtttatcattgttactattattactattactatcattattagtgttggcattattagtattcttcttgtcatcttgttcagaaatatacagcaatgctatacctaatactaaatacattatcatgctcgcaatggctctaacaacaacgaaccaaaatgaatttggtcgtagcttttgttcacaaaaatacataaagaaatgtctacataaatctatggcgccattggctacttgaaatagcgccagtcctcctacagattttaatatagctgtataacatgacatttattcatcatcaaaagagacagagtcaccatctgtcatatttagattttttttcatgtgttcaaagtatcctctactcatttcattataatagtttatcatacttagaattttaggacggatcaatgagtaagacttgactagatcgtcagtagtaatttgtgcatcgtctattctgcatccgcttcgtcgaataatgtatagcatcgctttgagattctccatagctatcaagtctttatacaatgacatggaaatatctgtgaatactttatacttctccaacatcgatgccttaacatcatcgcctactttagcattgaaaatacgttctattgtgtagatggatgtagcaagatttttaaacaacaatgccatcttacacgatgattgcctcaagtctccaatcttttgtttagaacgattagctacagagttcaacgcttggctgactagcatattattatctttagaaattgtattcttcaatgaggcgtttatcatatctgtgatttcgttagtcatattacagtctgactgggttgtaatgttatccaacatatcacctatggatacggtacacgtaccagcatttgtaataatcctatctaagatgttgtatggcattgcgcagaaaatatcttctcctgtaatatctccactctcgataaatctactcagattattcttaaatgccttattctctggagaaaagatatcagtgtccatcatttcattaatagtatacgcagaaaagataccacgagtatcaattctatccaagatacttatcggttccgagtcacagataatggtttcctctccttcgggagatcctgcatagaaatatctaggacaatagtttctatactgtctgtaactctgataatctctaaagtcactaactgataccatgaaattgagaagatcaaacgctgaagtaatcaatttttctgcctcgtttttactacaactagttttcatcaatgtagcgacgatgtattgtttagttactcttggtctaatactgatgatagagatattattgcttcccataatggatcttctagtagtcaccttaaagcccattgatgcgaatagcagatagataaagtcttggtatgactcctttctaatatagtacggactacctttgtcacccaactttatacccacataagccataacaacctctttaatagccgtttcatgaggtttatcagccatgagcctgagtagttggaagaatctcatgaatcccgtctcagaaagtcctatatgcatgatagatttatctttcctgggaaactctcgtatagtcatagatgaaatactcttcaaagtttctgaaataagattagtaacagtcttacctccgactactctgggtaacaaacatactctaataggtgttttctctgcggagataatatcagaaaggatagagcaataagtagtattattgtgattataaagaccgaatacataacaggtagaatttataaacatcatgtcctgaaggtttttagacttgtattcctcgtaatccataccgtcccaaaacatggatttggtaactttgatagccgtagatctttgttccttcgccaacaggttaaagaaattaataaagaatttgttgtttctatttatgtccacaaattgcacgtttggaagcgccacggttacattcactgcagcattttgaggatcgcgagtatgaagtacgatgttattgtttactggtatatctggaaagaaatctaccagtctaggaataagagattgatatcgcatagaaatagtaaagtttataatctcatcatcgaagagcattttgttaccattgtaataaatatccactctgtcatatgtataaatgaagtactgttcaaacatgatgagatgtttatatgttggcatagtagtgagatcgacgtttggtaatggcaatgtattaagattaactccataatgtctagcagcatctgcgatgttataagcgtcgtcaaagcggggtcgatcttgtattgttatatattgtctaacacctataagattatcaaaatcttgtctgcttaatacaccgttaacaatttttgccttgaattcttttattggtgcattaataacatccttatagaggatgttaaacaaataagtgttatcaaagttaagatctggatatttcttttctgctagaacatccattgagtcggagccatctggtttaatataaccaccgataaatctagctctgtattctgtatccgtcaatctaatattaagaaggtgttgagtgaaaggtggaagatcgtaaaagctgtgagtattaatgataggattagtttccgaactaatgttaattggggtattaataatatctatatttccagcgttaagtgtaacattaaacagttttaattcacgtgacgtggtatcaattaaataattaatgcccaatttggatatagcagcctgaagctcatcttgtttagttacggatcctaatgagttattaagcaatatatcgaacggatgaacgaaggttgttttaagttggtcacatactttgtaatctagacatagatgcggaagaacggtagaaactatacgaaataaatattcagagtcctctaattgatcaagagtaactattgacttaataggcatcatttatttagtattaaatgacgaccgtaccagtgacggatatacaaaacgatttaattacagagttttcagaagataattatccatctaacaaaaattatgaaataactcttcgtcaaatgtctattctaactcacgttaacaacgtggtagatagagaacataatgccgccgtagtgtcatctccagaggaaatatcctcacaacttaatgaagatctatttccagatgatgattcaccggccactattatcgaacgagtacaacctcatactactattattgacgatactccacctcctacttttcgtagagagttattgatatcggaacaacgtcaacaacgagaaaaaagatttaatattacagtatcgaaaaatgctgaagcaataatggaatctagatctatgataacttctatgccaacacaaacaccatccttgggagtagtttatgataaagataaaagaattcagatgttggaggatgaagtggttaatcttagaaatcaacgatctaatacaaaatcatctgataatttagataattttaccaaaatactatttggtaagactccgtataaatcaacagaagttaataagcgtatagccatcgttaattatgcaaatttgaacgggtctcccttatcagtcgaggacttggatgtttgttcagaggatgaaatagatagaatctataaaacgattaaacaatatcacgaaagtagaaaacaaaaaattatcgtcactaacgtgattattattgtcataaacattatcgagcaagcattgctaaaactcggatttgaagaaatcaaaggactgagtaccgatatcacttcagaaattatcgatgtggagatcggagatgactgcgatgctgtagcatcaaaactaggaatcggtaacagtccggttcttaatattgtattgtttatactcaagatattcgttaaacgaattaaaattatttaatttaatacattcccatatccagacaacaatcgtctggattaatctgttcctgtcgtctcataccggacgacatattaatctttttattagtaggcatctttttagatggtttctttttcccagcattaactgagtcgatacctagaagatcgtgattgatctctccgaccattccacgaacttctaattggccgtctctaacggtaccataaactattttaccagcattagtaacagcttggacaatctgaccatccatcgcattgtacgatgtagtagtaactgttgttctacgtctaggagcaccagaagtatttttggagcccttggaggctgatgtagaagaagacgaggattttgattttggtttacatgtaatacattttgaactctttgattttgtatcacatgcgccggcagtcacatctgtttgagaattaagattattgttgcctcctttgacggctgcatctccaccgatttgcgctagtagatttttaagctgtggtgtaatcttattaactgtttcgatataatcatcgtaactgcttctaacggctaaattttttttatccgccatttagaagctaaaaatatttttatttatacagaagatttaactagattatacaatgaactaatatgatccttttccagattatttacaaacttggtattttttggttctggaggaggcgaatttaaattcggacttggattcggattttgtgagttcttgatcttattatacatcgagtataggatggcgacggtaactgctacacaaataccgatcaacaaaagaataccaatcatttattgacaataacttcactattgatcaagtatgcaatatatcatcttttcactaaataagtagtaataatgattcaacaatgtcgagatatatggacgataataatttagttcatggaaatatcgctatgattggtatgaatgactccgctaactctgtggggcgcgcagtgctttccccacatagaataaattagcattccgactgtgataataataccaagtataaacgccataatactcaatactttccatgtacgagtgggactggtagacttactaaagtcaataaaggcgaagatacacgaaagaatcaaaagaatgattccagcgattagcacgccggaaaaataatttccaatcataagcatcatgtccatttaactaataaaaattttaaatcgccgaatgaacaaagtggaatataaaccatataaaaacaatagtttgtactgcaaaaataatatctatttttgttttcgaagatatggtaaaattaaatagtagtacacagcatgttataactaacagcagcaacggctcgtaattacttatcatttactagacgaaaaggtggtgggatattttcttgctcaaataatacgaatatatcacccatccattttatgcgatgtttatatactctaatctttaatagatctatagacgacgggtttaccaacaatatagattttatcgattcatctaatttaaacccttccttaaacgtgaatgatctattatctggcataacgatgaccctacctgatgaatcggacaatgtactgggccatgtagaataaattatcaacgaattatcgtctacgaacatttatatcatttgttttaattttaggacgcgaataaatagatataaaatagaaaataacagatattacaaccagtgttatggccgcgcccaaccaggtaggcagttttattttatcttttactacaggttctcctggatgtacgtcaccaacggcggacgtagttctagtacaattagacgtaagttccgcttgggaattttttaacgctaaagagttaacgttaatcgtgcacccaacgtatttacatctagttctttgaacatcttgattataatataaccattttctatctctagattcgtcggtgcactcatgtaaccaacataccctaggtcctaaatatttatctccggaattagattttggataattcgcgcaccaacaatttctatttcctttatgatcgttacaaaagacgtataatgccgtatccccaaaagtaaaataatcaggacgaataattctaataaactcagaacaatatctcgcatccatatgtttggagcaaatatcggaataagtagacatagccggtttccgttttgcacgtaaccattctaaacaattggggtttccaggatcgtttctacaaaatccagtcatgaaatcgtcacaatgttctgtcttgtaattattattaaatatttttggacagtgtttggtatttgtcttagaacaacattttgccacgctatcactatcgcccaggagataatccttttttataaaatgacatcgttgcccggatgctatataatcagtagcgtgttttaaatccttaatatattcaggagttacctcgttctgataatagattaatgatccaggacgaaatttgaaagaactacatggttctccatgaattaatacatattgtttagcaaattcaggaactataaaactactacaatgatctatcgacataccatctatcaaacaaaacttgggtttaatttctcccggagatgtttcataatagtacgtataactttcttctgcaaacttaacagctctattatattcaggataattaaaacctaattccatatatttgtctcgtatatctgctattcctggtgctattttgattctattaagagtaacagctgcccccattcttaataatcgtcagtatttaaactgttaaatgttggtatatcaacatctaccttatttcccgcagtataaggtttgttgcaggtatactgttcaggaatggttacatttatacttcttctatagtcctgtctttcgatgttcatcacatatgcaaagaacagaataaacaaaataatgtaagaaataatattaaatatctgtgaattcgtaaatacattgattgccataataattacagcagctacaatacacacaatagacattcccacagtgttgccattacctccacgatacatttgagttactaagcaataggtaataactaagctagtaagaggcaatagaaaagatgagataaatatcatcaatatagagattagaggagggctatatagagccaagacgaacaaaatcaaaccgagtaacgttctaacatcattatttttgaagattcccaaataatcattcattcctccataatcgttttgcatcatacctccatctttaggcataaacgattgctgctgttcctctgtaaataaatctttatcaagcactccagcacccgcagagaagtcgtcaagcatattgtaatatcttaaataactcatttatatattaaaaaatgtcactattaaagatggagtataatctttatgccgaactaaaaaaaatgacttgtggtcaacccctaagtctttttaacgaagacggggatttcgtagaagttgaaccgggatcatcctttaagtttctgatacctaagggattttacgcctctccttccgtaaagacgagtctagtatttgaaacattaacaacgaccgataataaaatcactagtatcaatccaacaaatgcgccaaagttatatcctcttcaacgcaaagtcgtatctgaagtagtttctaatatgaggaaaatgatcgaatcaaaacgtcctctatacattactcttcacttggcgtgtggatttggtaagactattaccacgtgttatcttatggctacacacggtagaaaaaccgtcatttgcgtacccaataaaatgttaatacatcaatggaagacacaggtagaggcagtcggattggaacataagatatccatagatggagtaagtagtctattaaaggaactaaagactcaaagtccggatgtattaatagtagtcagtagacatctgacaaacgatgccttttgtaaatatatcaataagcattatgatttgttcatcttggatgaatcacatacgtataatctgatgaacaatacagcagttacaagatttttagcgtattatcctccgatgatgtgttattttttaactgctacacctagaccagctaaccgaatttattgtaacagtattattaatattgccaagttatccgatctaaaaaaaactatctatgcggtagatagtttttttgagccatattccacagacaatattagacatatggtaaaacgactagatggaccatctaataaatatcatatatataccgagaagttattatctgtagacgagcctagaaatcaacttattcttaataccctggtagaagaattcaagtcaggaactattaatcgcattttagttattactaaactacgtgaacatatggtattattctacaaacgattattagatcttttcggaccagaggttgtatttataggagacgcccaaaatagacgtactccagatatggtcaaatcaatcaaggaactaaatagatttatattcgtatccaccttattttattccggtactggtttagatattcctagtttggattcgttgttcatttgctcggcagtaatcaacaatatgcaaatagagcaattactagggagggtatgtcgagaaacagaactattagataggacggtatatgtatttcctaacacatccatcaaagaaataaagtacatgataggaaatttcatgcaacgaattattagtctgtctgtagataaactaggatttaaacaagaaagttatcggaaacatcaagaatccgatcccacttctgtatgtacaacatcctccagagaagaacgtgtattaaatagaatatttaactcgcaaaatcgttaagaagtttaagcgacgatccgcatgctgcgcaggccagtgtattacccctcatagtattaatataatccaatgatacttttgtgatgtcggaaatcttaaccaatttagactgacaggcagaacacgtcatgcaatcatcatcgtcatcgataactgtagtcttgggcttctttttgcggctcttcattccggaacgcacattggtgctatccatttaggtagtaaaaaataagtcagaatatgccctataacacgatcgtgcaaaacctggtatatcgtctctatctttatcacaatatagtgtatcgacatctttattattattgacctcgtttatcttggaacatggaatgggaacatttttgttatcaacggccacctttgccttaattccagatgttgtaaaattataactaaacagtctatcatcgacacaaatgaaattcttgtttagacgtttgtagtttacgtatgcggctcgttcgcgtctcattttttcagatattgcaggtactataatattaaaaataagaatgaaataacataggattaaaaataaagttatcatgacttctagcgctgatttaactaacttaaaagaattacttagtctgtacaaaagtttgaaattttcagattctgcggctatagaaaagtataattctttggtagaatggggaacatctacttactggaaaataggcgtgcaaaaggtagctaatgtcgagacgtcaatatctgattattatgatgaggtaaaaaataaaccgtttaatattgatccgggctattacattttcttaccggtatattttgggagcgtctttatttattcgaagggtaaaaatatggtagaacttggatctggaaactcttttcaaataccagatgatatgcgaagtgcgtgtaacaaagtattagacagcgataacggaatagactttctgagatttgttttgttaaacaatagatggataatggaagatgctatatcaaaatatcagtctccagttaatatatttaaactagctagtgagtacggattaaacatacccaaatatttagaaattgaaatagaggaagacacattatttgacgacgagttatactctattatagaacgctcttttgatgataaatttccaaaaatatccatatcgtatattaagttgggagaacttaggcggcaagttgtagactttttcaaattctcgttcatgtatattgagtccatcaaggtagatcgtataggagataatatttttattcctagcgttataacaaaatcaggaaaaaagatattagtaaaagatgtagaccatttaatacgatctaaggttagagaacatacatttgtaaaagtaaaaaagaaaaacacattttccattttatacgactatgatgggaacggaacagaaactagaggagaagtaataaaacgaattatagacactataggacgagactattatgttaacggaaagtatttctctaaggttggtagtgcaggcttaaagcaattgactaataaattagatattaatgagtgcgcaactgtcgatgagttagttgatgagattaataaatccggaactgtaaaacgaaaaataaaaaaccaatcagcatttgatttaagcagagaatgtttgggatatccagaagcagattttataacgttagttaataacatgcggttcaaaatagaaaattgtaaggttgtaaatttcaatattgaaaatactaattgtttaaataacccgagtattgaaactatatatggaaactttaaccagttcgtctcaatctttaatatcgtcaccgatgtcaaaaaaagattattcgagtgaaataatatgcgcctttgatataggtgcaaaaaatcctgccagaactgttttagaagtcaaggataactccgttagggtattggatatatcaaaattagactggagttctgattgggaaaggcgcatagctaaagatttgtcacaatatgaatacactacagttcttctagaacgtcagcctagaaggtcgccgtatgttaaatttatctattttattaaaggctttttatatcatacatcggctgccaaagttatttgcgtctcgcctgtcatgtctggtaattcatatagagatcgaaaaaagagatcggtcgaagcatttcttgattggatggacacattcggattgcgagactccgttccggatagacgcaaattagacgatgtagcggatagtttcaatttggctatgagatacgtattagataaatggaatactaattatacaccttataataggtgtaaatctagaaattacataaaaaaaatgtaataacgttagtaacgccattatggataatctatttacctttctacatgaaatagaagatagatatgccagaactatttttaactttcatctaataagttgcgatgaaataggagatatatatggtcttatgaaagaacgcatttcctcagaggatatgtttgataatatagtatataataaagatatacatcctgccattaagaaactagtgtattgcgacatccaacttactaaacacattattaatcagaatacgtatccggtatttaacgattcttcacaagtgaaatgttgtcattatttcgacataaactcagataatagcaatattagctctcgtacagtagagatatttgagagggaaaagtcatctcttgtatcatatattaaaactaccaataagaagagaaaggtcaattacggcgaaataaagaaaactgttcatggaggcactaatgcaaattacttttccggtaaaaagtctgacgagtatctgagtactacagttagatccaacattaatcaaccttggatcaaaaccatttctaagagaatgagagtagatatcattaatcactctatagtaacgcgtggaaaaagctctatattacaaactatagaaattatttttactaatagaacatgtgtgaaaatattcaaggattctactatgcacattattctatccaaggacaaggatgaaaaggggtgtatacacatgattgacaaattattctatgtctattataatttatttctgttgttcgaagatatcatccaaaacgagtactttaaagaagtagctaatgttgtaaaccacgtacttacggctacggcattagatgagaaattattcctaattaagaaaatggctgaacacgatgtttatggagttagcaatttcaaaatagggatgtttaacctgacatttattaagtcgttggatcataccgttttcccctctctgttagatgaggatagcaaaataaagttttttaaggggaaaaagctcaatattgtagcattacgatctctggaggattgtataaattacgtgactaaatccgagaatatgatagaaatgatgaaggaaagatcgactattttaaatagcatagatatagaaacggaatcggtagatcgtctaaaagaattgcttctaaaatgaaaaaaaacactgattcagaaatggatcaacgactagggtataagtttttggtgcctgatcctaaagccggagttttttatagaccgttacatttccaatatgtatcgtattctaattttatattgcatcgattgcatgaaatcttgaccgtcaagcggccactcttatcgtttaagaataatacagaacgaattatgatagaaattagcaatgttaaagtgactcctccagattactcacctataatcgcgagtattaaaggtaagagttatgatgcattagccacgttcactgtaaatatctttaaagaggtaatgaccaaagagggtatatccatcactaaaataagtagttatgagggaaaagattctcatttgataaaaattccgctactaataggatacgggaataaaaatccacttgatacagccaagtatcttgttcctaatgtcataggtggagtctttatcaataaacaatctgtcgaaaaagtaggaattaatctagtagaaaagattacaacatggccaaaatttagggttgttaagccaaactcattcactttctcgttttcctccgtatcccctcctaatgtattaccgacaagatatcgccattacaagatatctctggatatatcacaattggaagcgttgaatatatcatcgacaaagacatttataacggtcaatattgttttgctgtctcaatatttatctagagtgagtctagaattcattagacgtagtttatcatacgatatgcctccagaagttgtctatctagtaaacgcgataatagatagtgctaaacgaattactgaatctattactgactttaatattgatacatacattaatgacctggtggaagctgaacacattaaacaaaaatctcagttaacgatcaacgagttcaaatatgaaatgctgcataactttttacctcatatgaactatacacccgatcaactaaagggattttatatgatatctttactaagaaagtttctctactgtatctaccacacttctagatatccagatagagattcgatggtttgtcatcgcatcctaacgtacggcaaatattttgagacgttggcacatgatgaattagagaattacataggcaacatccgaaacgatatcatgaacaatcacaagaacagaggcacttacgcggtaaacattcatgtactaacaactcccggacttaatcacgcgttttctagcttattgagtggaaagttcaaaaagtcagacggtagttatcgaacacatcctcactattcatggatgcagaatatttctattcctaggagtgttggattttatccggatcaagtaaagatttcaaagatgttttctgtcagaaaataccatccaagtcaatatctttacttttgttcatcggacgttccggaaagaggtcctcaggtaggtttagtatctcaattgtctgtcttgagttccattacaaatatactaacgtctgagtatttggatttggaaaagaaaatttgtgagtatatcagatcatattataaagatgatataagttactttgaaacaggatttccaatcactatagaaaatgctctagtcgcatctcttaatccaaatatgatatgtgattttgtaactgactttagacgtagaaaacggatgggattcttcggtaacttggaggtaggtattactttagttagggatcacatgaatgaaattcgcattaatattggagcgggaagattagtcagaccattcttggttgtggataacggagagctcatgatggatgtgtgtccggagttagaaagcagattagacgacatgacattctctgacattcagaaagagtttccgcatgtcatcgaaatggtagatatagaacaatttacttttagtaacgtatgtgaatcggttcaaaaatttagaatgatgtcaaaggatgaaagaaagcaatacgatttatgtgactttcctgccgaatttagagatggatatgtggcatcttcattagtgggaatcaatcacaattctggacccagagctattcttggatgtgctcaagctaaacaagctatctcttgtctgagttcggatatacgaaataaaatagacaatggaattcatttgatgtatccagagaggccaatcgtgattagtaaggctttagaaacttcaaagattgcggctaattgcttcggccaacatgttactatagcattaatgtcgtacaaaggtatcaatcaagaggatggaattatcatcaaaaaacaatttattcagagaggcggtctcgatatagttaccgcaaagaaacatcaagtagaaattccgttggaaaactttaataacaaagaaagagataggtctaacgcctattcaaaattagaaagtaatggattagttagactgaatgctttcttggaatccggagacgctatggcacgaaatatctcatcaagaactcttgaagatgattttgctagagataatcagattagcttcgatgtttccgagaaatataccgatatgtacaaatctcgcgttgaacgagtacaagtagaacttactgacaaagttaaggtacgagtattaaccatgaaagaaagaagacccattctaggagataaatttaccactagaacgagtcaaaagggaacagtcgcgtatgtcgcggatgaaacggaacttccatacgacgaaaatggtatcacaccagatgtcattattaattctacatccatcttctctagaaaaactatatctatgttgatagaggttattttaacagccgcatattctgctaagccgtacaacaataagggagaaaaccgacctgtctgttttcctagtagtaacgaaacatccatcgatacatatatgcaattcgctaaacaatgttatgagcattcaaatccgaaattgtccgatgaagaattatcggataaaatcttttgtgaaaagattctctatgatcctgaaacggataagccttatgcatccaaagtattttttggaccaatttattacttgcgtctgaggcatttaactcaggacaaggcaaccgttagatgtagaggtaaaaagacgaagctcattagacaggcgaatgagggacgaaaacgtggaggaggtatcaagttcggagaaatggagagagactgtttaatagcgcatggcgcagccaatactattacagaagttttaaaagactcagaagaggattatcaagatgtgtatgtttgtgaaaattgtggagacatagcagcacaaatcaagggtattaatacatgtcttagatgttcaaaacttaatctctctcctctcttaacaaaaattgataccacgcacgtatctaaagtatttcttactcaaatgaacgccagaggcgtaaaagtcaaattagatttcgaacgaaggcctccttcgttttataaaccattagataaagttgatctcaagccgtcttttctggtgtaatattctagtttggtagtagatacatatcaatatcatcaaattcgagatccgaattataaaatgggcgtggattgttaactatagaatcggacgtctgatattcgaaaatctgtggagttttaggttttggtggaggtgtaactgctacttgggatactgaagtctgatattcagaaagctgggggatgttctggttcgacatccaccgatggtgtcacatcactaatcggttcggtaacgtctgtggatggaggtgctacttctacagaacctgtagcctcagttgtcaacggagatacatttttaatgcgaggaaatgtataatttggtaatggtttctcatgtggatctgaagaagaggtaagatatctactagaaagataccgatcacgttctagttctcttttgtagaacttaactttttctttctcagcatctagttgatattccaacctcttcacgttactacgttcagattccaattcacgttcgcatgggttacctccgcagtttttacgagcgatttcacgttcagccttcatgcgtctctccttctctctatcgagtttatcagagcagtctttctgaaggcgatcgaactccataaatttctccaacgctttgattgtttccatagatttccgaagttcagcttttaggactgtgattctttttctttcgaattcacagctggatgtgcaaccgtttccattaccgccatctctaagtttcttttctagatcggcaacatttcatccccatgccttttacattcctcgagtctactgtcgtcgaaatatcgttccagctccttttcgacatcaataactttagcacgttgtctctcaagctctcttttgtagttatctgattccctggcacgtttaagatcttcatgcaattgagtcagctcttaacacaatctcttgcttcttcgtcatagtacttacaatcactatgggatccattgttaccacgtctacactcggcgagctcgcgtttaagagattcaatttcccgtttgtattggtccatgtttccattgctaccaccattagatttacaggctgctagttgtcgttcgagatcagaaatacgggttttcttggaattgatttcgtcgatgtacttggcatcgaaacacttattaagttctttttccaattctacgattttatttctttcgcgagtcaattccctcctgtagtaactatctgttttgtcagattcacgctctctacgtagactttcttgcaagttactaatttgttccctagcacgtccgagtttagttttatatgctgaatagagttctgattcatcctttgagcagatctctagcgatcgtttaagattcctgattctagtctttagcctatttacctcctcagaagatgttccgttaccgttgcgtttacactcgttaagctgtctatcaagatccatgattctatctctaagacgttgcatctctctttccatatcagcattgctttcattattacgtctgcagtcactcaactgtctttcaatatctgagattctatctctaagacgtcgcatctctctctgtttcggcattggtttcattattacgtctacagtcgttcaactgtctttcaagatctgatattctagattggagtctgctaatctctgtagcattttcacggcattcactcagttgtctttcaagatctgagattttagattggagtctgctaatctctgtaagatttcctcctccgctctcgatgcagtcggtcaacttattctctagttctctaatacgcgaacgcagtgcatcaacttcttgcgtgtcttcctggttgcgtgtacattcatcgagtctagattcgagatctctaacgcgtcgtcgttcttcctcaagttctctgcgtactacagaaagcgtgtccctatcttgttgatatttagcaatttctgattctagagtactgattttgcttacgtagttactaatagttgtcttggccttatcaagatcctccttgtatttgtcgcattccttgatatccctacgaagtctggacagttcccattcgacattacgacgtttatcgatttcagctcggagatcgtcatcgcgttgttttagccacatacgactgagttcaagttctcgttgacaagatccatctacttttccattcctaatagtatccagttccttttctagttctgaacgcatttctcgttccctatcaagcgattctctcaattctcggatagtcttcttatcaatttctaataaatctgaaccatcatctgtcccattttgaatatccctgtgttctttgatctcttttgtaagtcggtcgattctttcggttttataaacagaatccctttccaaagtcctaatcttactgagtttatcactaagttctgcattcaattcggtgagttttctcttggcttcttccaactctgttttaaactctccactatttccgcattcttcctcgcatttatctaaccattcaattagtttattaataactagttggtaatcagcgattcctatagccgttcttgtaattgtgggaacataattaggatcttctaatggattgtatggcttgatagcatcatctttatcattattagggggatggacaaccttaattggttggtcctcatctcctccagtagcgtgtggttcttcaataccagtgttagtaataggcttaggcaaatgcttgtcgtacgcgggcacttcctcatccatcaagtatttataatcgggttctacgtctgaatattcttttctaagagacgcgacttcgggagttagtagaagaactctgtttctgtatctatcaacgctggaatcaatactcaagttaaggatagcgaatacctcatcgtcatcatccgtatcttctgaaacaccatcatatgacatttcatgaagtctaacgtattgataaatagaatcagatttagtattaaacagatccttaacctttttagtaaacgcatatgtatattttagatctccagatttcataatatgatcacatgccttaaatgtcagtgcttccatgatataatctggaacactaatgggtgacgaaaaagatacagcaccatatgctacgttgataaataaatctgaaccactaagtagataatgattaatgttaaggaagaggaaatattcagtatatagatatgccttagcatcatatcttgtactaaacacgctaaacagtttattgatgtgatcaatttccaacagaataattagagcagcgggaataccaacaaacatattaccacatccgtattttctatgaatatcacatatcatgttaaaaaatcttgatagaagagcgaatatctcgtctgacttaatgagtcgtagttcagcagcaacataagtcataactgtaaatagaacatactttcctgtagtgttgattctagactccacatcaacaccattattaaaaatagttttatatacatctttaatctgctctccgttaatcgtcgaacgttctagtatacggaaacactttgatttcttatctgtagttaatgacttagtgatatcacgaagaatattacgaattacatttcttgtttttcttgagagacctgattcagaactcaactcatcgttccatagtttttttacctcagtggcgaaatctttggagtgcttggtacatttttcaataaggttcgtgacctccatttattataaaaaattttattcaaaacttaactacaatcgggtaattataaaatcgtagatctcccatgtggtggaatactaccatctatcgcatgtggatggacagtaggtaatggccatgggaacagtaatgtttgcatatttatctttcttgccagtattactgcatattgtcccaatgtttcgatgtgatgttctaacctatcaactgccgctgtatcacaacaatagtgtccgatgaaattaagattatgatccaatgtgtttaatatatgattatcaagtcttatacgatccgcgtcttttttgacaggatcaggttcttctacaggaagaagtttcggcctcttatgatattcatgtctgggaaacggtggtctagggtgaggctccggtatcggagtgggttttggattataatcatcatcgtctatgacatcatcttcgacttcgatatttattttgctatcttgatgatgtcctgtatcagttgcattttcagcactcgactgaatattagcgcattcattgtctattattaccatatttctaaacccaaaatgtatgtgttgaacatcagtactatcgttgatgagtcttatagcatgaattcgcttatcgttatcgggtttatcttctgtcaccttagcaattccttttttattaaactctacataatcatatccatttctattgtttgttctaatataaacgagtatagcatcattgctaaatttttcaatagtatcgaaaacagaatatcctaaaccatataatatatattcagggacactcaaactaaatgtccaggattctcctaaatacgtaaactttaatagtgcgaaatcattcaaaaatctaccacttatagatagatagtacataaatgcgtatagtagtctacctatctctttattatgaaaaccggcattacgatcatatatgtcgtgatatacctgtgatccgtttacgttaaaccataaatacatgggtgatcctataaacatgaatttatttctaattctcagagctatagttaattgaccgtgtaatatttgcttacatgcatacttgatacgctcattaataaaatttttatcattgctcgttatctcagaatcgtatatataaggagtaccatcgtgattcttaccagatattatacaaaatactatatataaaatatattgaccaacgttagtaatcatataaatgtttaacgttttaaattttgtattcaatgatccattatcatacgctagcatggtcttatgatattcattctttaaaatataatattgtgttagccattgcattggggctcctaatggagattttttattctcatccattttaggataggctttcataaagtccctaataacttcgtgaataatgtttctatgttttctactgatgcatgtatttgcttcgatttttttatcccatgtttcatctatcatagatttaaacgcagtaatgctcgcaacattaacatcttgaaccgttggtacaattccgttccataaatttataatgttcgccatttatataactcattttttgaatatacttttaattaacaaaagagttaagttactcatatgggcgccgtccagtctgaacatcaatctttttagccagagatatcatagccgctcttagagtttcagcgtgattttccaacctaaatagaacttcatcgttgcgtttacaacacttttctatttgttcaaactttgttgttacattagtaatctttttttccaaattagttagccgttgtttgagagtttcctcattgtcgtcttcatcggctttaacaattgcttcgcgtttagcctctggctttttagcagcctttgtagaaaaaaattcagttgctggaattgcaagatcgtcatctccggggaaaagagttccgtccatttaaagtacagattttagaaactgacactctgcgttatttatatttggtacaacacatggattataaatatcgatgttaataacatcagaaaatgtaaagtctatacattgttgcatcgtgttaaattttctaatggatctagtattattgggtccaacttctgcctgaaatccaaatatggaagcggatacaaaaccgtttcctggataaaccacacatctccacttttgctttacatcagaaattgtgtcgttgacatcttgaactctcctatctaatgccggtgttccacctatagattttgaatattcgaatgctgcatgagtagcattaaattccttaatattgccataattttcatatattgagtaaccctggataaaaagtaaacacaccgcagccgtcgctaccacaataaaaaaaattgatagagagttcatttataatctattagaagctgacaaaatttttttacacgcatcagacaatgctttaataaatagttcaacatctacttttgtcatatcgaaccgatggtatgattctaacctagaattacatccgaaaaagttgactatgttcatagtcattaagtcattaacaaacaacattccagactctggattataagacgatactgtttcgtcacaattacctaccttaatcatgtgattatgaatattggctattagagcaccttctaagaaatctataatatctttgaaacacgatttaaaatcaaaccacgaatatacttctacgaagaaagttagtttacccataggagaaataactataaatggagatctaaatacaaaatccggatctatgatagttttaacattattatattctctattaaatacctccacatctaaaaatgttaattttgaaactatgtcttcgtttattaccgtacctgaactaaacgctataagctctattgtttgagaactctttaaacgatattcttgaaatacatgtaacaaagtttcctttaactcggtcggtttatctaccatagttacagaatttgtatccttatctataatataataatcaaaatcgtataaagttatataattatcgcgttcagattgggatcttttcaaatagactaaaaaccccatttctctagtaagtatcttatgtatatgtttgtaaaatatcttcatggtgggaatatgctctaccgcagttagccattcctcattgacagcggtagatgtattagacaaaactattccaatgtttaacaagggccattttacgagattattaaatccttgtttgataaatgtagccaatgagggttcgagttcaacgacgattgaattctcttcccgcggatgctgcatgatgaacgacgggatgttgttcgattgatttggaattctttttcgactttttgtttatattaaatattttaaaatttatagcggatagcaattcatgtaccacggataatgtagacgcgtattgcgcatcgatatctttattattagataaatttatcaataaatgtgagaagtttgcctcgttaaggtcttccatttaaatattatataaacatttgtgtttgtatcttattcgtcttttatggaatagttttttactagtaaagctgcaattacacactttgtccgtaaaacataaatataaacaccagcttttatcaatcgttccaaaaagtcgacggcggacatttttaacatggcatctattttaaatacacttaggtttttggaaaaaacatcattttataattgtaacgattcaataactaaagaaaagattaagattaaacataagggaatgtcatttgtattttataagccaaagcattctaccgttgttaaatacttgtctggaggaggtatatatcatgatgatttggttgtattggggaaggtaacaattaataatctaaagatgatgctattttacatggatttatcatatcatggagtgacaagtagtggagcaatttacaaattgggatcgtctatcgatagactttctctaaataggactattgttacaaaagttaataattatgatgatacattttttgacgacgatgattgatcgctattgcacaattttgtttttttactttctaatatagcgtttagattctttttcatgtgcgaatattgatttactaaaatatcgatgtttaacttttgttctatgacgtccttatcagcggtatcggtacatatacgtaattcaccttcacaaaatacggagtcttcgataataatagccaatcgattattggatctagctgtctgtatcatattcaacatgtttaatatatcctttcgtttcccctttacaggcatcgatcgtagcatattttccgcgtctgagatggaaatgttaaaactacaaaaatgcgtaatgttagcccgtcctaatattggtacgtgtctataagtttggcatagtagaataatagacgtgtttaaatgccttccaaagtttaagaattctattagagtattgcattttgatagtttatcacctacatcatcaaaaataagtaaaaagtgtgctgattttttatgattttgtgcgacagcaatacatttttctatgttacttttagttcgtatcagattatattctagagattcctgactactaacgaaattaatatgatttggccaaatgtatccatcataatctgggttataaacgggtgtaaacaagaatatatgtttatattttttaactagtgtagaaaacagagatagtaaatagatagtttttccagatccagatcctcccgttaaaaccattctaaacggcatttttaataaattttctcttgaaaattgtttttcttggaaacaattcataattatatttacagttactaaattaatttgataataaatcaaaatatggaaaactaaggtcgttagtagggaggagaacaaagaaggcacatcgtgacataaataacatttattatcatgatgacaccagaaaacgacgaagagcagacatctgtgttctccgctactgtttacggagacaaaattcaaggaaagaataaacgcaaacgcgtgattggtctatgtattagaatatctatggttatttcactactatctatgattaccatgtccgcgtttctcatagtgcgcctaaatcaatgcatgtctgctaacgaggctgctattactgacgccgctgttgccgttgctgctgcatcatctactcatagaaaggttgcgtctagcactacacaatatgatcacaaagaaagctgtaatggtttatattaccagggttcttgttatatattacattcagactaccagttattctcggatgctaaagcaaattgcactgcggaatcatcaacactacccaataaatccgatgtcttgattacctggctcattgattatgttgaggatacatggggatctgatggtaatccaattacaaaaactacatccgattatcaagattctgatgtatcacaagaagttagaaagtatttttgtgttaaaacaatgaactaatatttatttttgtacattaataaatgaaatcgcttaatagacaaactgtaagtaggtttaagaagttgtcggtgccggccgctataatgatgatactctcaaccattattagtggcataggaacatttctgcattacaaagaagaactgatgcctagtgcttgcgccaatggatggatacaatacgataaacattgttatttagatactaacattaaaatgtctacagataatgcggtttatcagtgtcgtaaattacgagccagattgcctagaccggatactagacatctgagagtattgtttagtattttttataaagattattgggtaagtttaaaaaagaccaatgataaatggttagatattaataatgataaagatatagatattagtaaattaacaaattttaaacaactaaacagtacgacggatgctgaagcgtgttatatatacaagtctggaaaactggttaaaacagtatgtaaaagtactcaatctgtactatgtgttaaaaaattctacaagtgacaacaaaaaatgaattaataataagtcgttaacgtacgccgccatggacgccgcgtttgttattactccaatgggtgtgttgactataacagatacattgtatgatgatctcgatatctcaatcatggactttataggaccatacattataggtaacataaaaactgtccaaatagatgtacgggatataaaatattccgacatgcaaaaatgctactttagctataagggtaaaatagttcctcaggattctaatgatttggctagattcaacatttatagcatttgtgccgcatacagatcaaaaaataccatcatcatagcatgcgactatgatatcatgttagatatagaagataaacatcagccattttatctattcccatctattgatgtttttaacgctacaatcatagaagcgtataacctgtatacagctggagattatcatctaatcatcaatccttcagataatctgaaaatgaaattgttgtttaattcttcattctgcatatcagacggcaatggatggatcataattgatgggaaatgcaatagtaattttttatcataaaagttgtaaagtaaataataaaacaataaatattgaactagtagtacgtatattgagcaatcagaaatgatgctggtacctcttatcacggtgaccgtagttgcgggaacaatattagtatgttatatattatatatttgtaggaaaaagatacgtactgtctataatgacaataaaattatcatgacaaaattaaaaaagataaagagttctaattccagcaaatctagtaaatcaactgatagcgaatcagactgggaggatcactgtagtgctatggaacaaaacaatgacgtagataatatttctaggaatgagatattggacgatgatagcttcgctggtagtttaatatgggataacgaatccaatgtcatggcgcctagcacagaacacatttacgatagtgttgctggaagcacgctgctaataaataatgatcgtaatgaacagactatttatcagaacactacagtagtaattaatgagacggagactgttgaagtacttaatgaagataccaaacagaatcctaactattcatccaatcctttcgtaaattataataaaaccagtatttgtagcaagtcaaatccgttcattacagaactcaacaataaatttagtgagaataatccgtttagacgagcacatagcgatgattatcttaataagcaagaacaagatcatgaacacgatgatatagaatcatcggtcgtatcattggtgtgattagtttcctttttataaaattgaagtaatatttagtattattgctgccgtcacgttgtacaaatggagatattccctgtattcggcatttctaaaattagcaattttattgctaataatgactgtagatattatatagatacagaacatcaaaaaattatatctgatgagatcaatagacagatggatgaaacggtacttcttaccaacatcttaagcgtagaagttgtaaatgacaatgagatgtaccatcttattccccatagactatcgactattatactctgtattagttctgtcggaggatgtgttatctctatagataatgacatcaatgacaaaaatattctaacatttcccattgatcatgctgtaatcatatccccactgagtaaatgtgtcgtagttagcaagggtcctacaaccatattggttgttaaagcggatatacccagcaaacgattggtaacatcgtttacaaacgacatactgtatgtaaacaatctgtcactgattaattatttgccgttgtctgtattcattattagacgagtcaccgactatttggatagacacatatgcgatcagatatttgcgaataataagtggtattccattataaccatcgacgataagcaatatcctattccatcaaactgtataggtatgtcctctgccaagtacataaattctagcatcgagcaagatactttaatacatgtttgtaacctcgagcatccattcgacttagtatacaaaaaaatgcagtcgtacaattctgtacctatcaaggaacaaatattgtacggtagaattgataatataaatatgagcattagtatttctgtggattaatagatttctagtatggggatcattaatcatctctaatctctaaatacctcataaaacgaaaaaaaagctattatcaaatactgtacggaatggattcattctcttctctttttatgaaactctgttgtatatctactgataaaactggaagcaaaaaatctgataaaaagaataagaataagatcaaggattatatggaacacgattattataaaataacaatagttcctggttcctcttccacgtctactagctcgtggtattatacacatgcctagtaatagtctctttgcgttgacggaaagcagactagaaataacaggctaaaatgttcagacaccataatagttcccaacccagataataacagagtaccatcaacacattcctttaaactcaatcccaaacccaaaaccgttaaaatgtatccggccaattgatagtagataatgaggtgtacagcgcatgataatttacacagtaaccaaaatgaaaatactttagtaattataagaaatatagatggtaacgtcatcatcaacaatccaataatatgccggagagtaaacattgacggataaaacaaaaatgctccgcataactctatcatggcaataacacaaccaaatacttgtaagattcctaaattagtagaaaatacaacggatatcgatgtataagtgatctcgagaaataataagaataaagtaatgcccgtaaagataaacatcaacattgtttggtaatcattaaaccaattagtatgaagttgaactaatttcacagtagattttattccagtgttatcctcgcatgtatacgtacctggtaagatatctttatattctataatcaatgagacatcactatccgataacgaatgaagtctagcactagtatgccatttacttaatatggtcgtcttggaagttttattataagttaaaatatcatggttgtccaatttccatctaatatactttgtcggattatctatagtacacggaataatgatggtattattacatgctgtatactctatagtctttgtagatgttataatcataaaagtacagaggtatatcaacgatattctaactcttgacattttttatttatttaaaatgatacctttgttatttattttattctattttgctaacggtatcgaatggcataagtttgaaacgagtgaagaaataatttctacttacttattagacgacgtattatacacgggtgttaatggggcggtatacacattttcaaataataaactaaacaaaactggtttagctaatactaattatatcacaacatctataaaagtagaggatgcggataaggatacattagtatgcggaaccaataacggaaatcccaaatgttggaaaatagacggttcagacgacccaaaacatagaggtagaggatacgctccttatcaaaatagcaaagtaacgataatcagtcacaacggatgtgtactatctgacataaacatatcaaaagaaggaattaaacgatggagaagatttgacggaccatgtggttatgatttatacacggcggataacgtaattccaaaagatggtttacgaggagcattcgtcgataaagatggtacttatgacaaagtttacattcttttcactgatactatcggctcaaagagaattgtcaaaattccgtatatagcacaaatgtgcctaaacgacgaaggtggtccatcatcattgtctagtcatagatggtcgacgtttctcaaagtcgaattagaatgtgatatcgacggaagaagttatagacaaattattcattctagaactataaaaacagataatgatacgatactatatgtattcttcgatagtccgcattatgtacctattctatgaataccattaaacaatctttttctacgtcaaaattggaaggatatacaaagcaattgccgtctccagctcctggtatatgtctaccagctggaaaagttgttccacataccacgtttgaagtcatagaaaaatataatgtactagatgatattataaagcctttatctaaccaacctatcttcgaaggaccgtctggtgttaaatggttcgatataaaggagaaggaaaatgaacatcgggaatatagaatatacttcataaaagaaaattctatatattcgttcgatacaaaatctaaacaaactcgtagctcgcaagtcgatgcgcgactattttcagtaatggtaacttcgaaaccgttatttatagcagatatagggataggagtaggaatgccacaaatgaaaaaaatacttaaaatgtaatcttaatcgagtacaccacacgacaatgaacaaacataagacagattatgctggttatgcttgctgcgtaatatgcggtctaattgttggaattatttttacagcgacactattaaaagttgtagaacgtaaattagttcatacaccatcaatagataaaacgataaaagatgcatatattagagaagattgtcctactgactggataagctataataataaatgtatccatttatctactgatcgaaaaacctgggaggaaggacgtaatgcatgcaaagctctaaatccaaattcggatctaattaagatagagactccaaacgagttaagttttttaagaagcattagacgcggatattgggtaggagaatccgaaatattaaaccagacaaccccatataattttatagctaagaatgccacgaagaatggaactaaaaaacggaaatatatttgtagcacaacgaatactcccaaactgcattcgtgttacactatataacaattacactacatttttatcataccactacttcggttagatgttttagaaaaaaataaatatcgccgtaccgttcttgtttttataaaaataacaattaacaattatcaaattttttctttaatattttacgtggttgaccattcttggtggtaaaataatctcttagtgttggaatggaatgctgtttaatgtttccacactcatcgtatattttgacgtatgtagtcacatcgtttacgcaatagtcagactgtagttctatcatgcttcctacattagaaggaggaacagttttaaagtctcttggttttaatctattaccgttagttttcatgaaatcctttgttttatccacttcacattttaaataaatgtccactatacattcttctgttaattttactagatcgtcatgggtcatagaatttataggttccgtagtccatggatccaaactagcaaacttcgcgtatacggtatcgcgattagtgtatacaccaactgtatgaaaattaagaaaacagtttaatagatcaacagaaatatttaatcctccgtttgatacagatgcaccatatttatggattttggattcacacgttgtttgtctgaggggttcgtctagcgttgcttctacataaacttctattcccatatattctttattgtcagaatcgcataccgatttatcatcatacactgtttgaaaactaaatggtatacacatcaaaataacaaatactaacgagtacattctgcaatattgttatcgtaattggaaaaatagtgttcgagtgagttggattatgtgagtattggattgtatattttattttatattttgtaataagaataaaatgctaatgtcaagtttattccaatagatgtcttattaaaaacatatataataaataacaatggctgaatggcataaaattatcgaggatatctcaaaaaataataagttcgaggatgccgccatcgttgattacaagactacaaagaatgttctagctgctattcctaacagaacatttgccaagattaatccgggtgaaattattcctctcatcactaatcgtaatattctaaaacctcttattggtcagaaatattgtattgtatatactaactctctaatggatgagaacacgtatgctatggagttgcttactgggtacgcccctgtatctccgatcgttatagcgagaactcataccgcacttatatttttgatgggtaagccaacaacatccagacgtgacgtgtatagaacgtgtagagatcacgctacccgtgtacgcgcaactggtaattaaaataaaaagtaatattcatatgtagtgtcaattttaaatgatgatgatgaaatggataatatccatattgacgatgtcaataatgccggtattggcatacagctcatcgatttttagatttcattcagaggatgtggaattatgttatgggcatttgtattttgataggatctataatgtagtaaatataaaatataatccgcatattccatatagatataattttattaatcgcacgttaaccgtagatgaactagacgataatgtcttttttacacatggttattttttaaaacacaaatatggttcacttaatcctagtttgattgtctcattatcaggaaacttaaaatataatgatatacaatgctcagtaaatgtatcgtgtctcattaaaaatttggcaacgagtacatctactatattaacatctaaacataagacttattctctacatcggtccacgtgtattactataataggatacgattctattatatggtataaagatataaatgacaagtataatggcatctatgattttactgcaatatgtatgctaatagcgtctacattgatagtgaccatatacgtgtttaaaaaaataaaaatgaactcttaattatgctatgctattagaaatggataaaatcaaaattacggttgattcaaaaattggtaatgttgttaccatatcgtataacttggaaaagataactattgatgtcacacctaaaaagaaaaaagaaaaggatgtattattagcgcaatcagttgctgtcgaagaggcaaaagatgtcaaggtagaagaaaaaaatattatcgatattgaagatgacgatgatatggatgtagaaagcgcataatacgatctataaaaataagtatataaatactttttatttactgtactcttactgtgtagtggtgataccctactcgattatttttttaaaaaaatacttattctgattcttctagccatttccgtgttcgttcgaatgccacatcgacgttaaagataggggagtagttgaaatctagttctgcattgttggtacgcacctcaaatgtagtgttggatatcttcaacgtatagttgttgagtagtgatggttttctaaatagaattctcttcatatcattcttgcacgcgtacatttttagcatccatcttggaattctagatccttgttctattcccaatggtttcatcaatagaagattaaacatatcgtacgaacacgatggagagtaatcgtagcaaaagtaagcatttcctttaatctcagatcccggatactggatatattttgcagccaacacgtgcatccatgcaacatttcctacatatacccggctatgcaccgcgtcatcatcgactgtacgatacataatgttaccgtgttgcttacattgctcgtaaaagactttcgtcaatttgtctccttctccgtaaattccagtgggtcttaggcaacaagtatacaattttgctccattcatgattacggaattattggctttcataaccagttgctcggccatacgtttactttttgcgtatacatgtcctggtgatatatcataaagggtatgctcatggccgatgaatggatcaccgtgtttatttggtcctattgcttccatgctactagtatagatcaaatacttgattcctaggtccacacaagctgccaatatagtctgtgttccataatagtttactttcatgatttcattatcggtgtattttccaaatacatccactagagcagccgtatgaataatcagatttaccccatctagcgcttctctcaccttatcaaagtcgtttatatcacattgtatatagtttataaccttaactttcgaggttattggttgtggatcttctacaatatctatgactctgatttcttgaacatcatctgcactaattaacagttttactatatacctgcctagaaatccggcaccaccagtaaccgcgtacacggccattgctgccactcataatatcagactacttattctattttactaaataatggctgtttgtataatagaccacgataatatcagaggagttatttactttgaaccagtccatggaaaagataaagttttaggatcagttattggattaaaatccggaacgtatagtttgataattcatcgttacggagatattagtcaaggatgtgattccataggcagtccagaaatatttatcggtaacatctttgtaaacagatatggtgtagcatatgtttatttagatacagatgtaaatatatctacaattattggaaaggcgttatctatttcaaaaaatgatcagagattagcgtgtggagttattggtatttcttacataaatgaaaagataatacattttcttacaattaacgagaatggcgtttgatatatcagttaatgcgtctaaaacaataaatgcattagtttacttttctactcagcaaaataaattagtcatacgtaatgaagttaatgatacacactacactgtcgaatttgatagggacaaagtagttgacacgtttatttcatataatagacataatgacaccatagagataagaggggtgcttccagaggaaactaatattggttgcgcggttaatacgccggttagtatgacttacttgtataataagtatagttttaaactgattttagcagaatatataagacacagaaatactatatccggcaatatttattcggcattgatgacactagatgatttggctattaaacagtatggagacattgatctattatttaatgagaaacttaaagtagactccgattcgggactatttgactttgtcaactttgtaaaggatatgatatgttgtgattctagaatagtagtagctctatctagtctagtatctaaacattgggaattgacaaataaaaagtataggtgtatggcattagccgaacatatatctgatagtattccaatatctgagctatctagactacgatacaatctatgtaagtatctacgcggacacactgagagcatagaggataaatttgattattttgaagacgatgattcgtctacatgttctgccgtaaccgacagggaaacggatgtataattttttttatagcgtgaaggatatgataaaaaatataattgttgtatttatcccattccaatcaccttatatgattctgtaacacaatgaaggagtctcatagatgtatagaggtcagatactggtttgataaactgtttattccacatgagtatgtttgactttatggttagacccgcatactttaacaaatcactgaaaattggagttaggtattgacctctcagaatcagttgccgttctggaacattaaatgtattttttatgatatactccaacgcatttatgtgggcatacaacaagtcattactaatggagtattccaagagttttagttgtctagtatttaacaagagaagagatttcaacagactgtttatgaactcgaatgccgcctcattgtcgcttatattgatgatgtcgaattctcccaatatcatcaccgatgagtagctcatcttgttatcgggatccaagttttctaaagatgtcattaaaccctcgatcatgaatggatttatcatcatcgtttttatgttggacatgagcttagtccgtttgtccacatctatagacgacgatttctgaattattttatatatccctctctttaactccaggaacttgtcaggatggtctactttaatatgttctcgtctaagagatgaaaatctttggatggttgcacgcgacttttctctaaaggatgacgttgcccaagatcctctcttaaatgaatccatcttatccttggacaagatggacagtctattttccttagatggtttaatatttttgttacccatgatctataaaggtagacctaatcgtctcggatgacctatatatttattttcagttttattatacgcataaattgtaaaaaatatgttaggtttacaaaaatgtctcgtggggcattaatcgtttttgaaggattggacaaatctggaaaaacaacacaatgtatgaacatcatggaatctataccggcaaacacgataaaatatcttaactttcctcagagatccactgtcactggaaagatgatagatgactatctaactcgtaaaaaaacctataatgatcatatagttaatctattattttgtgcaaatagatgggagtttgcatcttttatacaagaacaactagaacagggaattactttaatagttgatagatacgcgttctctggagtagcgtatgccgccgctaaaggcgcgtcaatgactctcagtaagagttatgaatctggattgcctaaacccgacttagttatattcttggaatctggtagcaaagaaattaatagaaacgtcggcgaggaaatttatgaagatgttacattccaacaaaaggtattacaagaatataaaaaaatgattgaagaaggagatattcattggcaaattatttcttctgaattcgaggaagatgtaaagaaggagttgattaagaatatagttatagaggctatacacacggttactggaccagtggggcaactgtggatgtaatagtgaaattacattttttataaatagatgttagtacagtgttataaatggatgaagcatattactctggcaacttggaatcagtactcggatacgtgtccgatatgcataccgaactcgcatcaatatctcaattagttattgccaagatagaaactatagataatgatatattaaacaaggacattgtaaattttatcatgtgtagatcaaacttggataatccatttatctctttcctagatactgtatatactattatagatcaagagaactatcagaccgaattgattaattcattagacgacaatgaaattatcgattgtatagttaataagtttatgagcttttataaggataacctagaaaatatagtagatgctatcattactctaaaatatataatgaataatccagattttaaaactacgtatgccgaagtactcggttccagaatagccgatatagatattaaacaagtgatacgtgagaatatactacaattgtctaatgatatccgcgaacgatatttgtgaaaaatattaaaaaaaaatactttttttattaaatgacgtcgcttcgcgaatttagaaaattatgctgtgatatatatcacgcatcaggatataaagaaaaatctaaattaattagagactttataacagatagggatgataaatatttgatcattaagctattgcttcccggattagacgatagaatttataacatgaacgataaacaaattataaaattatatagtataatatttaaacaatctcaggaagatatgctacaagatttaggatacggatatataggagacactattaggactttcttcaaagagaacacagaaatccgtccacgagataaaagcattttaactttagaagacgtggatagtttcttaactacgttatcatccgtaactaaagaatcgcatcaaataaaattattgactgatatcgcatccgtttgtacatgtaatgatttaaaatgtgtagtcatgcttattgataaagatctaaaaattaaagcgggtcctcggtacgtacttaacgctattagtcctaatgcctatgatgtgtttagaaaatctaataacttgaaagagataatagaaaatgcatctaaacaaaatctagactctatatctatttctgttatgactccaattaatcccatgttagcggaatcgtgtgattctgtcaataaggcgtttaaaaaatttccatcaggaatgtttgcggaagtcaaatacgatggtgaaagagtacaagttcataaaaataataacgagtttgccttctttagtagaaacatgaaaccagtactctctcataaagtggattatctcaaagaatacataccgaaagcatttaaaaaagctacgtctatcgtattggattctgaaattgttcttgtagacgaacataatgtaccgctcccgtttggaagtttaggaatacacaaaaagaaagaatataaaaactctaacatgtgtttgttcgtgtttgactgtttgtactttgatggattcgatatgacggacattccattgtacgaacgaagatcttttctcaaagatgttatggttgaaatacccaatagaatagtattctcagagttgacgaatattagtaacgagtctcagttaactgacgtattggatgatgcactaacgagaaaattagaaggattggtcttaaaagatattaatggagtatacgaaccgggaaagagaagatggttaaaaataaagcgagactatttgaacgagggttccatggcagattctgccgatttagtagtactaggtgcttactatggtaaaggagcaaagggtggtatcatggcagtctttctaatgggttgttacgacgatgaatccggtaaatggaagacggttaccaagtgttcaggacacgatgataatacgttaagggttttgcaagaccaattaacgatggttaaaattaacaaggatcccaaaaaaattccagagtggttggtagttaataaaatctatattcccgattttgtagtagaggatccgaaacaatctcagatatgggaaatttcaggagcagagtttacatcttccaagtcccataccgcaaatggaatatccattagatttcctagatttactaggattagagaagataaaacgtggaaagaatctactcatctaaacgatttagtaaacttgactaaatcttaatagttacatacaaactgaaaattaaaataacactatttagttggtggtcgccatggatggtgttattgtatactgtctaaacgcgttagtaaaacatggcgaggaaataaatcatataaaaaatgatttcatgattaaaccatgttgtgaaagagtttgtgaaaaagtcaagaacgttcacattggcggacaatctaaaaacaatacagtgattgcagatttgccatatatggataatgcggtatccgatgtatgcaattcactgtataaaaagaatgtatcaagaatatccagatttgctaatttgataaagatagatgacgatgacaagactcctactggtgtatataattattttaaacctaaagatgttattcctgttatcatatctataggaaaggataaagatgtctgtgaactattaatctcatcagacatatcgtgtgcatgcgtggagttaaattcatatcacgtagccattcttcccatggatgtttccttttttaccaaaggaaatgcatcattgattattctcctgtttgatttctctatcgatgcagcacctctcttaagaagtgtaaccgataataatgttattatatctagacaccagcgtctacatgacgagcttccgagttccaattggttcaagttttacataagtataaagtccgactattgttctatattatatatggttgttgatggatctgtgatgcatgcgatagctgataatagaactcacgcaattattagcaaaaatatattagacaatactacgattaacgatgagtgtagatgctgttattttgaaccacagattaggattcttgatagagatgagatgctcaatggatcatcgtgtgatatgaacagacattgtattatgatgaatttacctgatgtaggcaaatttggatctagtatgttggggaaatatgaacctgacatgattaagattgctctttcggtggctggtaatttaataagaaatcgagactacattcccgggagacgaggatatagctactacgtttacggtatagcctctagataatttttttaagcacgaaataaaaaacataattttaaaccaatctatttcatactattttgtgtgatcaccatggacataaagatagatattagtatttctggtgataaatttacggtgactactaggagggaaaatgaagaaagaaaaaaatatctacctctccaaaaagaaaaaactactgatgttatcaaacctgattatcttgagtacgatgacttgttagatagagatgagatgtttactattctagaggaatattttatgtacagaggtctattaggcctcagaataaaatatggacgactctttaacgaaattaaaaaattcgacaatgatgcggaagaacaattcggtactatagaagaactcaagcagaaacttagattaaattctgaagagggagcagataactttatagattatataaaggtacaaaaacaggatatcgtcaaacttactgtatacgattgcatatctatgataggattgtgtgcatgcgtggtagatgtttggagaaatgagaaactgttttctagatggaaatattgtttacgagctattaaactgtttattaatgatcacatgcttgataagataaaatctatactgcagaatagactagtatatgtggaaatgtcatagaaagttaaaagttaatgagagcaaaaatatataaggttgtattccatatttgttattttttctgtaatagttagaaaaatacattcgatggtctatctaccagattattatgtgttataaggtactttttctcataataaactagagtatgagtaagatagtgtttttcaaaacatataaatctaaaattgatggatgagatatacagctattaatttcgaaaatatattttaatctgataactttaaacatggatttttgatggtggtttaacgttttaaaaaaagattttgttattgtagtatatgataatattaaaagatggatataaagaatttgctgactgcatgtactattttttacattactacattggctacggcagatatacctactccgccaccaacgggtcatgtgacaagggagaatatcttgataagaggcataatcaatgttgtaatcggtgtccacctggagaatttgccaaggttagatgtaatggtaacgataacacaaaatgtgaacgctgcccacctcatacatataccacaatcccaattattctaatggatgtcatcaatgtagaaaatgcccaaccggatcatttgataaggtaaagtgtaccggaacacagaacagtaaatgttcgtgtcttcctggttggtattgtgctactgattcttcacagactgaagattgttgaaattgtgtaccaaaaaggagatgtccatgcggatactttggtggaatagatgaacaaggaaatcctatttgtaaatcgtgttgtgttggtgaatattgcgactacctacgtaattatagacttgatccatttcctccatgcaaactatctaaatgtaattaattatgattttgatgataatgttaccatacattatatcgctacttggttagtgtattattcagtatgaagacctattaataattacttatcttttgacgatcttgttataattataatataaaaatacttatggcatagtaactcataattgctgacgcgataaattcgtaataatctgttttgttcaaatttttataaggaatctacaggcataaaaataaaaatataatttataatatactcttacagcgcgccatcatgaataacagcagtgaattgattgctgttattaatggatttagaaatagtggacgattttgtgatattagtatagttattaatgatgaaaggataaacgctcataaactcatcctatctggagcctccgaatatttttccattctgttttccaataattttatcgattctaatgaatacgaagttaatctaagtcatttagattatcaaagcgttaacgatttgatcgattacatttatgggatacctttgagcctaactaacgataacgtgaaatatattctttcaaccgctgattttttacaaattggatctgctattacggagtgtgaaaattacatacttaaaaatctttgttctaaaaactgtatcgatttctacatatacgctgataaatataataacaagaaaatagaatcagcgtcgtttaacacaatattacaaaatattttgagactcatcaacgatgaaaactttaaatacttaacagaggaatcaatgataaaaattttaagcgatgatatgttaaatataaaaaatgaggattttgcaccactaattctcattaaatggttagagagtactcaacaatcatgcaccgtcgagttacttagatgcctcagaatatcattgctttccccacaagttataaaatcactttatagtcatcaactggttagttcaatctacgaatgtataacattcttaaacaatatagcattcttggatgaatcatttcctagataccatagcatcgagttgatatctatcggtataagtaattcgcatgataagatttccataaactgctacaatcataaaaaaaatacatgggaaatgatatcttcacgtagatataggtgtagtttcgcagtggccgtcctggataatattatctatatgatgggtggatatgatcagtccccgtatagaagttcaaaggttatagcgtacaatacatgtacaaattcttggatatatgatataccagagctaaaatatcctcgttctaattgtgggggactggctgatgacgaatacatttattgtataggcggcatacgcgatcaggattcatcgttgacatctagtattgataaatggaagccatcaaaaccatattggcagaagtatgctaaaatgcgcgaaccaaaatgtgatatgggggttgcgatgttaaacggattaatatatgtcatgggtggaatcgttaaaggtgacacgtgtaccgacgcactagagagtttatcagaagatggatggatgaagcatcaacgtcttccaataaaaatgtccaatatgtcgacgattgttcatgatggcaagatttatatatctggaggttacaacaatagtagtgtagttaatgtaatatcgaatctagtccttagctataatccgatatatgatgaatggaccaaattatcatcattaaacattcctagaattaatcccgctctatggtcagcgcataataaattatatgtaggaggaggaatatctgatgatgttcgaactaatacatctgaaacatacgataaagaaaaagattgttggacattggataatggtcacgtgttaccacgcaattatataatgtataaatgcgaaccgattaaacataaatatccattggaaaaaacacagtacacgaatgattttctaaagtatttggaaagttttataggtagttgatagaacaaaatacataattttgtaaaaataaatcactttttatactaatatgacacgattaccaatacttttgttactaatatcattagtatacgctacaccttttcctcagacatctaaaaaaataggtgatgatgcaactctatcatgtaatcgaaataatacaaatgactacgttgttatgagtgcttggtataaggagcccaattccattattcttttagctgctaaaagcgacgtcttgtattttgataattataccaaggataaaatatcttacgactctccatacgatgatctagttacaactatcacaattaaatcattgactgctagagatgccggtacttatgtatgtgcattctttatgacatcaactacaaatgacactgataaagtagattatgaagaatactccacagagttgattgtaaatacagatagtgaatcgactatagacataatactatctggatctacacattcaccggaaactagttctaagaaacctgattatatagataattctaattgctcgtcggtattcgaaatcgcgactccggaaccaattactgataatgtagaagatcatacagacaccgtcacatacactagtgatagcattaatacagtaagtgcatcatctggagaatccacaacagacgagactccggaaccaattactgataaagaagatcatacagttacagacactgtctcatacactacagtaagtacatcatctggaattgtcactactaaatcaaccaccgatgatgcggatctttatgatacgtacaatgataatgatacagtaccaccaactactgtaggcggtagtacaacctctattagcaattataaaaccaaggactttgtagaaatatttggtattaccgcattaattatattgtcggccgtggcaattttctgtattacatattatatatataataaacgttcacgtaaatacaaaacagagaacaaagtctagatttttgacttacataaatgtctgggatagtaaaatctatcatattgagcggaccatctggtttaggaaagacagccatagccaaaagactatgggaatatatttggatttgtggtgtcccataccactagatttcctcgtcctatggaacgagaaggtgttgattaccattacgttaacagagaggccatctggaagggaatagccgccggaaactttctagaacatactgagtttttaggaaatatttacggaacttctaaaacagctgtgaatacagcggctattaataatcgtatttgtgtgatggatctaaacatcgacggtgttagaagtcttaaaaatacgtacctaatgccttactcggtgtatataagacctacctctcttaaaatggttgagaccaagcttcgttgtagaaacactgaagctaacgatgagattcatcgtcgcgtgatattggcaaaaacggatatggatgaggccaacgaagcaggtctattcgacactattatcattgaagatgatgtgaatttagcatatagtaagttaattcagatactacaggaccgtattagaatgtattttaacactaattagagacttaagacttaaaacttgataattaataatataactcgtttttatatgtggctatttcaacgtctaatgtattagttaaatattaaaacttaccacgtaaaacttaaaatttaaaatgatatttcattgacagatagatcacacattatgaactttcaaggacttgtgttaactgacaattgcaaaaatcaatgggtcgttggaccattaataggaaaaggtggattcggtagtatttatactactaatgacaataattatgtagtaaaaatagagcccaaagctaacggatcattatttaccgaacaggcattttatactagagtacttaaaccatccgttatcgaagaatggaaaaaatctcacaatataaagcacgtaggtcttatcacgtgcaaggcatttggtctatacaaatccattaatgtggaatatcgattcttggtaattaatagattaggtgcagatctagatgcggtgatcagagccaataataatagattaccaaaaaggtcggtgatgttgatcggaatcgaaatcttaaataccatacaatttatgcacgagcaaggatattctcacggagatattaaagcgagtaatatagtcttggatcaaatagataagaataaattatatctagtggattacggattggtttctaaattcatgtctaatggagaacatgttccatttataagaaatccaaataaaatggataacggtactctagaatttacacctatagattcgcataaaggatacgttgtatctagacgtggagatctagaaacacttggatattgtatgattagatggttgggaggtatcttgccatggactaagatatctgaaacaaagaattgtgcattagtaagtgccacaaaacagaaatatgttaacaatactgcgactttgttaatgaccagtttgcaatatgcacctagagaattgctgcaatatattaccatggtaaactctttgacatattttgaggaacccaattatgacgagtttcggcacatattaatgcagggtgtatattattaagtgtggtgtttggtcgatgtaaaatttttgtcgataaaaattaaaaaataacttaatttattattgatctcgtgtgtacaaccgaaatcatggcgatgttttacgcacacgctctcggtgggtacgacgagaatcttcatgcctttcctggaatatcatcgactgttgccaatgatgtcaggaaatattctgttgtgtcagtttataataacaagtatgacattgtaaaagacaaatatatgtggtgttacagtcaggtgaacaagagatatattggagcactgctgcctatgtttgagtgcaatgaatatctacaaattggagatccgatccatgatcaagaaggaaatcaaatctctatcatcacatatcgccacaaaaactactatgctctaagcggaatcgggtacgagagtctagacttgtgtttggaaggagtagggattcatcatcacgtacttgaaacaggaaacgctgtatatggaaaagttcaacatgattattctactatcaaagagaaggccaaagaaatgaatgcacttagtccaggacctatcattgattaccacgtctggataggagattgtatctgtcaagttactgctgtggacgtacatggaaaggaaattatgagaatgagattcaaaaagggtgcggtgcttccgatcccaaatctggtaaaagttaaacttggggagaatgatacagaaaatctttcttctactatatcggcggcaccatcgaggtaaccacctctctggaagacagcgtgaataatgtactcatgaaacgtttggaaactatacgccatatgtggtctgtcgtatatgatcattttgatattgtgaatggtaaagaatgctgttatgtgcatacgcatttgtctaatcaaaatcttataccgagtactgtaaaaacaaatttgtacatgaagactatgggatcatgcattcaaatggattccatggaagctctagagtatcttagcgaactgaaggaatcaggtggatggagtcccagaccagaaatgcaggaatttgaatatccagatggagtggaagacactgaatcaattgagagattggtagaggagttcttcaatagatcagaacttcaggctggtgaatcagtcaaatttggtaattctattaatgttaaacatacatctgtttcagctaagcaactaagaacacgtatacggcagcagcttcctttatactctcatcttttaccaacacaaagggtggatatttgttcattggagttgataataatacacacaaagtaattggattcacggtgggtcatgactacctcagactggtagagaatgatatagaaaagcatatcaaaagacttcgtgttgtgcatttctgtgagaagaaagaggacatcaagtacacgtgtcgattcatcaaggtatataaacctggggatgaggctacctcgacatacgtgtgcgctatcaaagtggaaagatgctgttgtgctgtgtttgcagattggccagaatcatggtatatggatactaatggtatcaagaagtattctccagatgaatgggtgtcacatataaaattttaattaatgtaatagagaacaaataataaggttgtaatatcatatagacaataactaacaattaattagtaactgttatctcttttttaactaaccaactaactatatacctattaatacatcgtaattatagttcttaacatctattaatcattaattcgcttctttaattttttataaactaacattgttaattgaaaagggataacatgttacagaatataaattatatatggatttttttaaaaaggaaatacttgactggagtatatatttatctcttcattatatagcacgcgtgttttccaatttttccacatcccatataatacaggattataatctcgttcgaacatacgagaaagtggataaaacaatagttgattttttatctaggttgccaaatttattccatattttagaatatggggaaaatattctacatatttattctatggatgatgctaatacgaatattataattttttttctagatagagtattaaatattaataagaacgggtcatttatacacaatctcaggttatcatcatccattaatataaaagaatatgtatatcaattagttaataatgatcatccagataataggataagactaatgcttgaaaatggacgtagaacaagacattttttgtcctatatatcagatacagttaatatctatatatgtattttaataaatcatggattttatatagatgccgaagacagttacggttgtacattattacatagatgtatatatcactataagaaatcagaatcagaatcatacaatgaattaattaagatattgttaaataatggatcagatgtagataaaaaagatacgtacggaaacacaccttttatcctattatgtaaacacgatatcaacaacgtggaattgtttgagatatgtttagagaatgctaatatagactctgtagactttaatagatatacacctcttcattatgtctcatgtcgtaataaatatgattttgtaaagttattaatttctaaaggagcaaatgttaatgcgcgtaatagattcggaactactccattttattgtggaattatacacggtatctcgcttataaaactatatttggaatcagacacagagttagaaatagataatgaacatatagttcgtcatttaataatttttgatgctgttgaatctttagattatctattatccagaggagttattgatattaactatcgtactatatacaacgaaacatctatttacgacgctgtcagttataatgcgtataatacgttggtctatctattaaacagaaatggtgattttgagacgattactactagtggatgtacatgtatttcggaagcagtcgcaaacaacaacaaaataataatggaagtactattgtctaaacgaccatctttgaaaattatgatacagtctatgatagcaattactaaacataaacagcataatgcagatttattgaaaatgtgtataaaatatactgcgtgtatgaccgattatgatactcttatagatgtacagtcgctacagcaatataaatggtatattttaaaatgtttcgatgaaatagatatcatgaagagatgttatataaaaaataaaactgtattccaattagttttttgtatcaaagacattaatactttaatgagatacggtaaacatccttctttcgtgaaatgcactagtctcgacgtatacggaagtcgtgtacgtaatatcatagcatctattagatatcgtcagagattaattagtctattatccaagaagctggatcctggagataaatggtcgtgttttcctaacgaaataaaatataaaatattggaaaactttaacgataacgaactatccacatatctaaaaatcttataaacactattaaaatataaaatctaagtaggataaaatcacactacatcattgtttccttttagtgctcgacagtgtatactatttttaacactcataaataaaaatgaaaacgatttccgttgttacgttgttatgcgtactacctgctgttgtttattcaacatgtactgtacccactatgaataacgctaaattaacgtctaccgaaacatcgtttaatgataaacagaaagttacgtttacatgtgatcagggatatcattcttcggatccaaatgctgtctgcgaaacagataaatggaaatacgaaaatccatgcaaaaaaatgtgcacagtttctgattacatctctgaattatataataaaccgctatacgaagtgaattccaccatgacactaagttgcaacggcgaaacaaaatattttcgttgcgaagaaaaaaatggaaatacttcttggaatgatactgttacgtgtcctaatgcggaatgtcaacctcttcaattagaacacggatcgtgtcaaccagttaaagaaaaatactcatttggggaatatatgactatcaactgtgatgttggatatgaggttattggtgcttcgtacataagttgtacagctaattcttggaatgttattccatcatgtcaacaaaaatgtgatatgccgtctctatctaatggattaatttccggatctacattttctatcggtggcgttatacatcttagttgtaaaagtggttttacactaacggggtctccatcatccacatgtatcgacggtaaatggaatcccgtactcccaatatgtgtacgaactaacgaagaatttgatccagtggatgatggtcccgacgatgagacagatttgagcaaactctcgaaagacgttgtacaatatgaacaagaaatagaatcgttagaagcaacttatcatataatcatagtggcgttaacaattatgggcgtcatatttttaatctccgttatagtattagtttgttcctgtgacaaaaataatgaccaatataagttccataaattgctaccgtaaatataaatccgttaaaataatgaataattaataacgaacaagtatcaaaagattaaagaattatagctagaatcaattgagatgtcttcttcagtggatgttgatatctacgatgccgttagagcatttttactcaggcactattataacaagagatttattgtgtatggaagaagtaacgccatattacataatatatacaggctatttacaagatgcgccgttataccgttcgatgatatagtacgtactatgccaaatgaatcacgtgttaaacaatgggtgatggatacacttaatggtataatgatgaatgaacgcgatgtttctgtaagcgttggcaccggaatactattcatggaaatgtttttcgattacaataaaaatagtatcaacaatcaactaatgtatgatataattaatagcgtatctataattctagctaatgagagatatagaagcgcttttaacgacgatggtatatacatccgtagaaatatgattaacaagttgtacggatacgcatctctaactactattggcacgatcgctggaggtgtttgttattatctgttgatgcatctagttagtttgtataaataattatttcaatatactagttaaaattttaagattttaaatgtataaaaaactaataacgtttttatttgtaataggtgcattagcatcctattcgaataatgagtacactccgtttaataaactgagtgtaaaactctatatagatggagtagataatatagaaaattcatatactgatgataataatgaattggtgttaaattttaaagagtacacaatttctattattacagagtcatgcgacgtcggatttgattccatagatatagatgttataaacgactataaaattattgatatgtataccattgactcgtctactattcaacgcagaggtcacacgtgtagaatatctaccaaattatcatgccattatgataagtacccttatattcacaaatatgatggtgatgagcgacaatattctattactgcagagggaaaatgctataaaggaataaaatatgaaataagtatgatcaacgatgatactctattgagaaaacatactcttaaaattggatctacttatatatttgatcgtcatggacatagtaatacatattattcaaaatatgatttttaaaaatttaaaatatattatcacttcagtgacagtagtcaaataacaaacaacaccatgagatatattataattctcgcagttttgttcattaatagtatacacgctaaaataactagttataagtttgaatccgtcaattttgattccaaaattgaatggactggggatggtctatacaatatatcccttaaaaattatggcatcaagacgtggcaaacaatgtatacaaatgtaccagaaggaacatacgacatatccgcatttccaaagaatgatttcgtatctttctgggttaaatttgaacaaggcgattataaagtggaagagtattgtacgggactatgcgtcgaagtaaaaattggaccaccgactgtaacattgactgaatacgacgaccatatcaatttgtacatcgagcatccgtatgctactagaggtagcaaaaagattcctatttacaaacgcggtgacatgtgtgatatctacttgttgtatacggctaacttcacattcggagattctaaagaaccagtaccatatgatatcgatgactacgattgcacgtctacaggttgcagcatagactttgtcacaacagaaaaagtgtgcgtgacagcacagggagccacagaagggtttctcgaaaaaattactccatggagttcgaaagtatgtctgacacctaaaaagagtgtatatacatgcgcaattagatccaaagaagatgttcccaatttcaaggacaaaatggccagagttatcaagagaaaatttaataaacagtctcaatcttatttaactaaatttctcggtagcacatcaaatgatgttaccacttttcttagcatgcttaacttgactaaatattcataactaatttttattaatgatacaaaaacgaaataaaactgcatattatacactggttaacgcccttataggctctaaccattttcaagatgaggtccctgattatagtccttctgttcccctctatcatctactccatgtctattagacaatgtgagaagactgaagaggaaacatggggattgaaaatagggttgtgtataattgccaaagatttctatcccgaaagaactgattgcagtgttcatctcccaactgcaagtgaaggattgataactgaaggcaatggattcagggatatacgaaacaccgataaattataaaaaaagcaatgtgtccgctgtttccgttaataatactattttcgtaactggcggattattcataaataactctaatagcacgatcgtggttaacaatatggaaaaacttgacatttataaagacaaacaatggtcgattatagaaatgcctatggctagggtatatcacggcatcgactcgacatttggaatgttatattttgccggaggtctatccgttaccgaacaatatggtaatttagagaaaaacaacgagatatcttgttacaatcctagaacgaataagtggtttgatatttcatatactatttataagatatccatatcatcattgtgtaaactaaataacgtcttctatgtatttagtaaggacattggatatgtggaaaagtatgatggtgcatggaagttagtacatgatcgtctccccgctataaaggcattatcaacttctccttattgattgaaaatgaaaatataaatagtttttatgtatagcagtattaccctatagttttattgcttactactaacatggatacagatgttacaaatgtagaagatatcataaatgaaatagatagagagaaagaagaaatactaaaaaatgtagaaattgaaaataataaaaacattaacaagaatcatccaagtggatatattagagaagcactcgttattaataccagtagtaatagtgattccattgataaagaagttatagaatgtatctgtcacgatgtaggaatatagatcatatctactaatttttataatcgatacaaaacataaaaaacaactcgttattacatagcaggcatggaatccttcaagtattgttttgataacgatggcaagaaatggattatcggaaatactttatattctggtaattcaatactatataaggtcagaaaaaatttcactagttcgttctacaattacgtaatgaagatagatcacaaatcacacaagccattgttgtctgaaatacgattctatatatctgtattggatcctttgactatcgacaactggacacgggaacgtggtataaagtatttggctattccagatctgtatggaattggagaaaccgatgattatatgttcttcgttataaagaatttgggaagagtattcgccccaaaggatactgaatcagtcttcgaagcatgcgtcactatgataaacacgttagagtttatacactctcaaggatttacccatggaaaaatagaaccgaggaatatactgattagaaataaacgtctttcactaattgactattctagaactaacaaactatacaagagtggaaactcacatatagattacaacgaggacatgataacttcaggaaatatcaattatatgtgtgtagacaatcatcttggagcaacagtttcaagacgaggagatttagaaatgttgggatattgcatgatagaatggttcggtggcaaacttccatggaaaaacgaaagtagtataaaagtaataaaacaaaaaaaagaatataaaaaatttatagctactttctttgaggactgttttcctgaaggaaatgaacctctggaattagttagatatatagaattagtatacacgttagattattctcaaactcctaattatgacagactacgtaaactgtttatacaagattgaaattatattcttttttttatagagtgtggtagtgttacggatatttaatattagactatctctatcgcgctacacgaccaatatcgattactatggatatcttcagggaaatcgcatcttctatgaaaggagagaatgtattcatttctccagcgtcaatctcgtcagtattgacaatactgtattatggagctaatggatccactgctgaacagctatcaaaatatgtagaaaaggaggagaacatggataaggttagcgctcaaaatatctcattcaaatccataaataaagtatatgggcgatattctgccgtgtttaaagattcctttttgagaaaaattggcgataagtttcaaactgttgacttcactgattgtcgcactatagatgcaatcaacaagtgtgtagatatctttactgaggggaaaatcaatccactattggatgaaccattgtctcctgatacctgtctcctagcaattagtgccgtatactttaaagcaaaatggttgacgccattcgaaaaggaatttaccagtgattatcccttttacgtatctccgacggaaatggtagatgtaagtatgatgtctatgtacggcaaggcatttaatcacgcatctgtaaaggaatcattcggcaacttttcaatcatagaactgccatatgttggagatactagtatgatggtcattcttccagacaagattgatggattagaatccatagaacaaaatctaacagatacaaattttaagaaatggtgtaactctctggaagctacgtttatcgatgttcacattcccaagtttaaggtaacaggctcgtataatctggtggatactctagtaaagtcaggactgacagaggtgttcggttcaactggagattatagcaatatgtgtaattcagatgtgagtgtcgacgctatgatccacaaaacgtatatagatgtcaatgaagagtatacagaagcagctgcagcaacttgtgcactggtgtcagactgtgcatcaacaattacaaatgagttctgtgtagatcatccgttcatctatgtgattaggcatgttgatggaaaaattcttttcgttggtagatattgctctccgacaactaattgttaaccattttttttaaaaaatagaaaaaacatgtggtattagtgcaggtcgttattcttccaattgcaattggtaagatgacggccaactttagtacccacgtcttttcaccacagcactgtggatgtgacagactgaccagtattgatgacgtcagacaatgtttgactgaatatatttattggtcgtcctatgcataccgcaacaggcaatgcgctggacaattgtattccacactcctctcttttagagatgatgcggaattagtgttcatcgacattcgcgagctggtaaaaaatatgccgtgggatgatgtcaaagattgtgcagaaatcatccgttgttatataccggatgagcaaaaaaccatcagagagatttcggccatcatcggactttgtgcatatgctgctacttactggggaggtgaagaccatcccactagtaacagtctgaacgcattgtttgtgatgcttgagatgctcaattacgtggattataacatcatattccggcgtatgaattgatgagttgtacatcttgacattttcttctttcttctcttctccctttcccagaaacaaactttttttacccactataaaataaaatgagtatactacctgttatatttctttctatatttttttattcttcattcgttcagacttttaacgcgcctgaatgtatcgacaaagggcaatattttgcatcattcatggagttagaaaacgagccagtaatcttaccatgtcctcaaataaatacgctatcatccggatataatatattagatattttatgggaaaaacgaggagcggataatgatagaattataccgatagataatggtagcaatatgctaattctgaacccgacacaatcagactctggtatttatatatgcattaccacgaacgaaacctactgtgacatgatgtcgttaaatttgacaatcgtgtctgtctcagaatcaaatatagatcttatctcgtatccacaaatagtaaatgagagatctactggcgaaatggtatgtcccaatattaatgcatttattgctagtaacgtaaacgcagatattatatggagcgggcatcgacgccttagaaataagagacttaaacaacggacacctggaattattaccatagaagatgttagaaaaaatgatgctggttattatacatgtgttttagaatatatatacggtggcaaaacatataacgtaaccagaattgtaaaattagaggtacgggataaaataataccttctactatgcaattaccagatggcattgtaacttcaataggtagtaatttgactattgcatgcagagtatcgttgagacctcccacaacggatgcagacgtcttttggataagtaatggtatgtattacgaagaagatgatggggacggaaacggtagaataagtgtagcaaataaaatctatatgaccgataagagacgtgttattacatcccggttaaacattaatcctgtcaaggaagaagatgctacaacgtttacgtgtatggcgtttactattcctagcatcagtaaaacagttactgttagtataacgtgaatgtatgttgttacatttccatgtcaattgagtttataagaatttttatacattatcttccaacaagcaattgacgaacgtattgctatgattaactcccacgatactatgcatattattaatcattaacttgcagactatacctagagctattttgacatactcgtgttcttgtgtaattgcagtatctatattattaaagtacgtaaatctagctatagttttattatttaattttagataatataccgtctccttatttttaaaaattgccacatcctttattaaatcatgaatgggaatttctatgtcatagttaatatattgtgaacaacaagagcagatatctataggaaagggtggaatgcgatacattgatctatgtagttttaaaacacacgcaaactttgaagaatttatataaatcattccatcgatacatccttctatgttgagatgtatatatccaggaattcgtttattaatatcgggaaatgtataaactaaaacattgcccggaagcggagcttctaccggagttatatcagtttttaacttacaaaatgtaaccaatacctttgcatgacttgttttgttcggcaacgttagtttaaacttgacgaatggattaattacaatagcatgatccgcgcatctattaagtttttttactttaacgcccttgtatgtttttacagagactttatctaaatttctagtgcttgtatgtgttataaatataacgggatatagaactgaatcacctaccttagatacccaattacattttatcagatccagataataaacaaattttgtcgccctaactaattctatattgttatatattttacaattggttatgatatcatgtaataacttggaatctaacgcacatcgtcgtacgtttatacaattgtgatttagtgtagtatatctacacatgtatttttccgcgctatagtattctggactagtgataaaactatcgttatatctgtcttcaatgaactcatcgagatattgctctctgtcatattcatacacctgcataaactttctagacatcttacaatccgtgttattttaggatcatatttacatatttacgggtatatcaaagatgttagattagttaatgggaatcgtctataataatgaatattaaacaattatatgaggacttttaccacaaagcatcataaaaatgagtcgtcgtctgatttatgttttaaatatcaaccgcgaatcaactcataaaatacaagagaatgaaatatatacatattttagtcattgcaatatagaccatacttctacagaacttgattttgtagttaaaaactatgatctaaacagacgacaacctgtaactgggtatactgcactacactgctatttgtataataattactttacaaacgatgtactgaagatattattaaatcatggagtggatgtaacgatgaaaaccagtagcggacgtatgcctgtttatatattgcttactagatgttgcaatatttcacatgatgtagtgatagatatgatagacaaagataaaaaccacttattacatagagactattccaacctattactagagtatataaaatctcgttacatgttattaaaggaagaggatatcgatgagaacatagtatccactttattagataagggaatcgatcctaactttaaacaagacggatatacagcgttacattattattatttgtgtctcgcacacgtttataaaccaggtgagtgtagaaaaccgataacgataaaaaaggccaagcgaattatttctttgtttatacaacatggagctaatctaaacgcgttagataattgtggtaatacaccattccatttgtatcttagtattgaaatgtgtaataatattcatatgactaaaatgctgttgacttttaatccgaatttcgaaatatgtaataatcatggattaacgcctatactatgttatataacttccgactacatacaacacgatattcttgttatgttaatacatcactatgaaacaaatgttggagaaatgccgatagatgagcgtcgtataatcgtattcgagtttatcaaaacatattctacacgtcctgcagattcgataacttatttgatgaataggtttaaaaatatagatatttatacccgctatgaaggaaagacattattacacgtagcatgtgaatataataatacacacgtaatagattatcttatacgtatcaacggagatataaatgcgttaaccgacaataacaaacacgctacacaactcattatagataacaaagaaaattccccatataccattaattgtttactgtatatacttagatatattgtagataagaatgtgataagatcgttggtggatcaacttccatctctacctatcttcgatataaaatcatttgagaaattcatatcctactgtatacttttagatgacacattttacaatagacacgttaggaatcgcgattctaaaacgtatcgatacgcattttcaaaatacatgtcgtttgataaatacgatggtataataactaaatgtcataaagaaacaatattgctcaaactatccactgttctagacactacactatatgcagttttaagatgccataattcgaaaaagttaagaagatacctcaccgagttaaaaaaatataataacgataagtcctttaaaatatattctaatattatgaatgagagataccttaatgtatattataaagatatgtacgtgtcaaaggtatatgataaactatttcctgttttcacagataaaaattgtctactaacattactaccttcagaaattatatacgaaatattatacatgctgacaattaacgatctttataatatatcgtatccacctaccaaagtatagttgtatttttctcatgcgatgtgtgtaaaaaaactgatattatataaatattttagtgccgtataataaagatgacgatgaaaatgatggtacatatatatttcgtatcattattgttattgctattccacagttacgccatagacatcgaaaatgaaatcacagaattcttcaataaaatgagagatactctaccagctaaagactctaaatggttgaatccagcatgtatgttcggaggcacaatgaatgatatagccgctctaggagagccattcagcgcaaagtgtcctcctattgaagacagtcttttatcgcacagatataaagactatgtggttaaatgggaaaggctagaaaaaaatagacggcgacaggtttctaataaacgtgttaaacatggtgatttatggatagccaactatacatctaaattcagtaaccgtaggtatttgtgcaccgtaactacaaagaatggtgactgtgttcagggtatagttagatctcatattagaaaacctccttcatgcattccaaaaacatatgaactaggtactcatgataagtatggcatagacttatactgtggaattctttacgcaaaacattataataatataacttggtataaagataataaggaaattaatatcgacgacattaagtattcacaaacgggaaaggaattaattattcataatccagagttagaagatagcggaagatacgactgttacgttcattacgacgacgttagaatcaagaatgatatcgtagtatcaagatgtaaaatacttacggttataccgtcacaagaccacaggtttaaactaatactagatccaaaaatcaacgtaacgataggagaacctgccaatataacatgcactgctgtgtcaacgtcattattgattgacgatgtactgattgaatgggaaaatccatccggatggcttataggattcgattttgatgtatactctgttttaactagtagaggcggtattaccgaggcgaccttgtactttgaaaatgttactgaagaatatataggtaatacatataaatgtcgtggacacaactattattttgaaaaaacccttacaactacagtagtattggagtaaatatacaatgcatttttatatacattactgaataattattattattatttatatcgtatttgtgctataacgcgactatctaggtatttgtatctcaccgatagagaacatataaatgtagactctattaaacagttgtgtaaaatatcagatcctaatgcatgttatagatgtggatgtacggctttacatgagtacttttataattatagatcagtcaacggaaaatacaagtatagatacaacggttactatcaatattatttatctagcgattatgaaaattataatgaatattattatgatgattatgatagaactggtatgaacagtgagagtgataatatatcaatcaaaacagaatatgaattctatgatgaaacacaagatcaaagtacacaactagtaggttacgacattaaactcaaaaccaatgaggatgattttatggctatgatagatcagtgggtgtccatgattatatagatgaatcaattaataaagtagtatatggaagagagtctcacgtaagatggcgggatatatggcaagaacataatgatggcgtatacagtataggaaaggagtgcatagataatatatacgaagacaaccataccgtagacgaattctacaagatagacagcgtatcagatgtagatgacgcggaacacatatctccgataactaatgatgtatctacacaaacatgggaaaagaaatcagagttagatagatacatggaaatgtatcctcgtcatagatatagtaagcattctgtctttaagggattttctgacaaagttagaaaaaatgatttagacatgaatgtggtaaaagaattactttctaacggtgcatctctaacaattaaggatagcagtaataaggatccaataaccgtttattttcgaagaacgataatgaatttagaaatgattgatgaacgaaagtatatagtacactcctatctaaaaaattataaaaatttcgattatccatttttcaggaagttagttttgactaataaacattgtctcaacaattattataatataagcgacagcaaatatggaacaccgctacatatattggcgtctaataaaaaattaataactcctaattacatgaagttattagtgtataacggaaatgatataaacgcacgaggtgaagatacacaaatgcgaactccattacacaaatatttgtgtaaatttgtatatcataatattgaatatggtatccgatactataatgaaaagattatagacgcatttatagagttaggagccgatctaactattccaaatgacgatggaatgataccagtagtttactgtatacactcaaatgccgaatatggttataacaatattactaacataaagataatacgtaaactacttaatcttagtagacgtgcgtcacataatctatttagagatcgagtcatgcacgattatataagtaatacatatattgatcttgagtgtttagatattattagatcgttggatggattcgatatcaatggttactttgaaggacgtacaccacttcattgcgctatacaacataacttcactcagattgctaagtacttattagatcgaggagctgatatagtcgtacccaacacattgattatacatcagtacatacagtaaatagcatagatatggaggaggatacaaatatttcaaataaagttataaggtacaacactgtcaataatatatgggaaacattacctaacttctggactggaactataaatccaggcgtggtctcgcataaagatgatatatatgttgtatgcgacatcaaagatgaaaaaaatgttaaaacttgtatatttagatataacacgaatacgtataacggatgggaattggtcacgacgacagaaagcagattatcagctctgcatactattctttataacaataccataatgatgttacattgttatgaatcgtatatgttacaagatacatttaatgtgtacactcgcgaatggaatcatatgtgtcatcaacattcgaatagttatatcatgtacaatatactacccatctactaaatataatagaataaaataaatgagtatgatcattttagataacgattgattttatcattaccgcttcattcttatattctttgcttacggaacctatatttagaaacatctactaacgattttttatgcttgcattattaatggtatgtaatatgattgattgtgtacgcaataccaatttgttaagtatgaatacggggtacaaacataaactgaagtttaacattatttatttatgatatatatcgttattgtttggtctataccatggatatctttaaagaactaatcttaaaacacacggatgaaaatgttttgatttctccagtttctattttatctactttatctattctaaatcatggagcagctggttctacagctgaacaactatcaaaatatatagagaatatgaatgagaatacacccgatgacaataatgacatggacgtagatattccgtattgtgcgacactagctaccgcaaataaaatatacggtagcgatagtatcgagttccacgcctccttcctacaaaaaataaaagacgattttcaaactgtaaactttaataatgctaaccaaacaaaggaactaatcaacgaatgggttaagacaatgacaaatggtaaaattaattccttattgactagtccgctatccattaatactcgtatgacagttgttagcgccgtccattttaaagcaatgtggaaatatccattttctaaacatcttacatatacagacaagttttatatttctaagaatatagttaccagtgttgatatgatggtgagcactgagaataacttgcaatatgtacatattaatgaattattcggaggattctctattatcgatattccatacgagggaaactctagtatggtaattatactaccggacgacatagaaggtatatataacatagaaaaaaatataacagatgaaaaatttaaaaaatggtgtggtatgttatctactaaaagtatagacttgtatatgccaaagtttaaagtggaaatgacagaaccgtataatctggtaccgattttagaaaatttaggacttactaatatattcggatattatgcagattttagcaagatgtgtaatgaaactatcactgtagaaaaatttctacatacgacgtttatagatgttaatgaggagtatacagaagcatcggccgttacaggagtatttatgactaacttttcgatggtatatcgtacgaaggtctacataaaccatccattcatgtacatgattaaagacaacacaggacgtatactttttatagggaaatactgctatccgcaataaatataaacaaatagacttttatcacgtttatctatgtctaaatattacaaatagtaatagtataaactaaagctgataatacttaaaaaaataataatatcatttacaattaatagtataaactaaaaattaaacaaatcgttattataagtaatatcaaaatgatgatatacggattaatagcgtgtcttatattcgtgacttcatccatcgctagtccactttatattcccgttattccacccatttcggaagataaatcgttcaatagtgtagaggtattagtttccttgtttagagatgaccaaaaagactatacggtaacttctcagttcaataactacactatcgataccaaagactggactatcggcgtactatccacacctgatggtttggatataccattgactaatataacttattggtcacggtttactataggtcgtgcattgttcaaatcagagtctgaggatattttccaaaagaaaatgagtattctaggtgtttctatagaatgtaagaagtcgtcgacattacttacttttttgaccgtgcgtaaaatgactcgagtatttaataaatttccagatatggcttattatcgaggagactgtttaaaagccgtttatgtaacaatgacttataaaaatactaaaactggagagactgattacacgtacctctctaatggggggttgcctgcatactatcgtaatggggtcgatggttgattattgattagtatattccttattctttttattcacacaaaaagaacatttttataaacatgaaaccactgtctaaatgtaattatgatcttgatttatagatgaagatcagcctttagaggattttaaccagtatgtttaatatgaaaaaaataaacataacatattttgagattaagcgctattgtgcaagattatattagaatcaaattaatctttcatacgagaaaaataacgacatacgtcgtcaacaaattaaactttttatttattagttaactagcttatagaacttgctcattgttatgtttctaaaacgggtacgacatataggacaattatccgacgcaccggtttctcttcgtgttttatgccatatattgatgcatgttatgcaaaatatatgagtacacgaatccaataaaccaaagtatctatcgttttgagtaaacaacttcatagcaaattccacattctttttctttacttactctatacacgtcctcgtatttatccagtattttgatgatatccaactcagaaatggttgttgtattattgggtgtattgggagtataggtattattagctatgtaccaatttaccaaccctcttaatattgattgaacaatcacatcggttatccaatcaataaccacattaataactaaattgtagtgtatatatagaccatatatgtttctatttttttgacagttacgtatagtttcagtaagttttgattgttgtattcctgtatctctagataagttagtcatatagtcccttccggcgatacgttttttccaagcccgaaattgattagccaaatgtgtatttatttttgtgatattgatataatatgtaatgttattaatatttcggataatgcatactgttagtcttatatcatttggttcatctatgtattgtaatattgttacatgatctatagatgatgtattgattttggcaggatcgaattccatatccgcgactaaacagtgaaaaaaatgtaaatactttttaaattttaaattagtaaaacttttttttattttttatgattccaaaaatactgaatacaaagtcctaaattataaatatggagatcatactaccacaacttattattatgcatactcagccggtgtaatagatagatatatataattctattacaccggcagacaattaccgatcggtatttgtcgttaccaacataccgtataatatgtaatatacaattccataacccattgacagttgttatacatcaaaattgcaattcttttgattacgatgttataagaatgtagttaattgatgtatgatgttaatgtgtcctctttcctcttataacatcgtaatcaaaaacttttttataatatatacctaataatgtgtcttaatagttctcgtgattcgtcaaacaatcattcttataaaatataataaagcaacgtaaaacacataaaaataagcgtaactaataagacaatggatatttacgacgataaaggtctacagactattaaactgtttaataatgaatttgattgtataaggaatgacatcagagaattatttaaacatgtaactgattccgatagtatacaacttccgatggaagacaattctgatattatagaaaatatcagaaaaatactatatagacgattaaaaaatgtagaatgtgttgacatcgatagtacaataacttttatgaaatacgatccaaatgatgataataagcgtacgtgttctaattgggtacccttaactaataactatatggaatattgtctagtaatatatttggaaacaccgatatgtggaggcaaaataaaattataccaccctacaggaaatataaagtcggataaggatattatgtttgcaaagactctagactttaaatcaaagaaagtgttaactggacgtaaaacaattgccgttctagacatatccgtttcatataatagatcaatgactactattcactacaacgacgacgttgatatagatatacatactgataaaaatggaaaagagttatgttattgttatataacaatagatgatcattacttggttgatgtggaaactataggagttatagtcaatagatctggaaaatgtctgttagtaaataaccatctaggtataggtatcgttaaagataaacgtataagcgatagttttggagatgtatgtatggatacaatatttgacttttctgaagcacgagagttattttcattaactaatgatgataacaggaatatagcatgggacactgataaactagacgatgatacagatatatggactcccgtcacagaagatgattacaaatttctttctagactagtattgtatgcaaaatctcaatcggatactgtattcgactattatgttcttactggtgatacggaaccacccactgtattcattttcaaggtaactagattttactttaatatgccgaaataaaaaatttttgtataatatctagaggtagaggtattgtttagataaatacaaataacatagatacatcgcatacttagcatttttataaatatacataagacatacactttatacattttttttgtaaaaatactcataaaaaaatttataaaaattatggcacaaccatatcttgtataggtagtttagttcgtcgagtgaacctataaacagataatagacaacacgtaataataataatgcctactaatacaagcataataccgggagatgggatatatgacgttgtagtgtttgggttttctgaacgttgatagtctactaatactacatgctgacatctaatgcctgtataaccatgagagcatctacaatacataccgtcaatatctctagcgtggatacagtcaccgtgtaaacaatatccatctccctctggaccgcataatctgatagctggaatatctgttgtagcgtttgtaatttctggcgatgtcgtttcgatagcgttaccactatcggcgaatgatctgattatcatagcagcgaacaacaacatcagatatttcatcgacatttttgatggattttgtgtttatgctgtttctcagtgtgtgtttatgacaagattgggaattttatattattaattcagtaatataaactaataatatattgttaattgtgtaaataatataaaaataacaatacaatattgaatgtgttgctgttaaaaatgatcataaacacggagtttattttatatgtctcgcataaacattactaaaaaaatatattgttctgtttttctttcacatctttaattatgaaaaagtaaatcattatgagatggacgagattgtacgcatcgttcgcgacagtatgtggtacatacctaacgtatttatggacgacggtaagaatgaaggtcacgtttctgtcaacaatgtctgtcatatgtatttcacgttctttgatgtggatacatcgtctcatctgtttaagctagttattaaacactgcgatctgaataaacgaggtaactctccattacattgctatacgatgaatacacgatttaatccatctgtattaaagatattgttacaccacggcatgcgtaactttgatagcaaggatgaaaaaggacaccactatcaatcgataacaagatctttgatatactaacggacaccattgatgactttagtaaatcatccgatctattgctgtgttatcttagatataaattcaatgggagcttaaactattacgttctgtacaaaggatccgacgaggatgaactcacttctcttcattactactgtaaacacatatccacgttctacaaaagcaattattacaagttaagtcacactaagatgcgagccgagaagcgattcatctacgcgataatagattatggagcaaacattaacgcggttacacacttaccttcaacagtataccaaacatagtcctcgtgtggtgtatgctcttttatctcgaggagccgatacgaggatacgtaataatcttgattgtacacccatcatggaacgattgtgcaacaggtcatattctcataatgttactcaattggcacgaacaaaaggaagaaggacaacatctactttatctattcataaaacataatcaaggatacactctcaatatactacggtatctactagataggttcgacattcagaaagacgaatactataataccgcctttcaaaattgtaacaacaatgttgcctcatacatcggatacgacatcaaccttccgactaaagacggtattcgacttggtgtttgaaaacagaaacatcatatacaaggcggatgttgtgaatgacatcatccaccacagactgaaagtatctctacctatgattaaatcgttgttctacaagatgtctctccctacgacgattactacgtaaagaagataatagcctactgcctattaagggacgagtcattcgcggaactacatagtaaattctgtttaaacgaggactataaaagtgtatttatgaaaaatatatcattcgataagatagattccatcatcgtgacataagtcgccttaaagagattcgaatctccgacaccgacctgtatacggtatcacagctatcttaaagccatacattcagacagtcacatttcatttcccatgtacgacgatctcatagaacagtgccatctatcgatggagcgtaaaagtaaactcgtcgacaaagcactcaataaattagagtctaccatcggtcaatctagactatcgtatttgcctccggaaattatgcgcaatatcatctaaacagtatgttgtacgaaaagaaccattacaaatattatccatgatagaaagaaaatatctatatgattggagaagtaggaaacaggaacaagacgacgattactacattattaaatcatgaagtccgtattatactcgtatatattgtttctctcatgtataataataaacggaagagatatagcaccgcatgcaccatccgatggaaagtgtaaagacaacgaatacaaacgccataatttgtgtccgggaacatacgcttccagattatgcgatagcaagactaacacacaatgtacgccgtgtggttcgggtaccttcacatctcgcaataatcatttacccgcttgtctaagttgtaacggaagacgcgaccgtgtaacacgactcacaatagaatctgtgaatgctctcccggatattattgtcttctcaaaggatcatccggatgcaaggcatgtgtttcccaaacaaaatgtggaataggatacggagtatccggagacgtcatctgttctccgtgtggtctcggaacatattctcacaccgtctcttccgcagataaatgcgaacccgtacccagtaatacctttaactatatcgatgtggaaattaatctgtatccagttaacgaccactaccggtctcagcgaatccatctcaacgtcggaactaactattactatgaatcataaagactgtaatcccgtatttcgtgaggaatacttctccgtccttaataaggtagcaacttcaggtttctttacaggagaaaggtgtgcactctgaatttcgagattaaatgcaataacaaaaatttttcctccaaacagttaacgaaagcaaagaatgatgacggtatcatgccgcattcggagactgtctatctagcgtcgacatctatatactatatagtaataccaatactcaagactacgaaactgatacaatctcttatcatgtgggtaatgttctcgatgtcgatagccatatgcccggtagttgcgatatacataaactgatcactaattccaaacccacccgctttttatagtaagtttttcacccataaataataaatacaataattaatttctcgtaaaagtagaaaatatattctaatttattgcacggtaaggaagtagaatcataaagaacagtactcaatcaatagcaatcatgaaacaatatatcgtcctggcatgcatgtgcctgccagtcttcagcaatcatcctcatcctcctcctcgtgtacggaagaagaaaacaaacatcatatgggaatcgatgttattatcaaagtcacaaagcaagaccaaacaccgaccaatgataagatttgccaatccgtaacggaaattacagagtccgagtcagatccagatcccgaggtggaatcagaagatgattccacatcagtcgaggatgtagatcctcctaccacttattactccatcatcggtggaggtctgagaatgaactttggattcaccaaatgtcctcagattaaatccatctcagaatccgctgatggaaacacagtgaatgctagattgtccagcgtgtccccaggacaaggtaaggactctcccgcgatcactcatgaagaagctctggctatgatcaaagactgtgaggtgtctatcgacatcagatgtagcgaagaagagaaagacagcgacatcaagacccatccagtactcgggtctaacatctctcataagaaagtgagttacgaagatatcatcggttcaacgatcgtcgatacaaaatgtgtcaagaatctagagtttagcgttcgtatcggagacatgtgcaaggaatcatctgaacttgaggtcaaggatggatttaagtatgtcgacggatcggcatctgaaggtgcaaccgatgatacttcactcatcgattcaacaaaactcaaagcgtgtgtctgaatcgataactctattcatctgaaattggatgagtagggttaatcgaacgattcaggcacaccacgaattaaaaaagtgtaccggacactatattccggtttgcaaaacaaaaatgttcttaactacattcacaaaaagttacctctcgcgacttcttctttttctgtctcaatagtgtgatacgattatgacactattcctattcctattcctatttcctttcagggtatcacaaaaatattaaacctctttctgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaatattaaacctctttctgatggtgtcactaaaatatttttattctcattctcattttctctttctctcttcaatggagtcataaaatatttttattctctttctctcttcgatggtctcacaaaaatattaaacctctttctgatggtgtcactaaaatatttttattctcattctcattttctctttctctcttcaatggagtcataaaatatttttattctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcactaaaatattttttattctctttctgatgcatcaactatttcttaaacaataacgtccaacaacatatactcgtcgagcttatcaacatcccctatgcccatctaggttaccagacaattgtatatcataaaataatgtttataatttacacgttaaaatcatataataaaacgtagatcgtataatattttttggtatataaatgatctagtaaaatccatgtaggggatactgctcacattttttctttggtacaaaatttcacacaagtttttatacagacaaattcttgtccatatattttaaaacattgacttttgtactaagaaaaatatctagatcaactatctctttctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctccttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctccttcgatggtctcacaaaaatattaaacctctttctgatggagtcgtaaaaaagttttatctctttctccttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggtctctataaagcgatcgatctttcttacactctagagtttcctacagtcatgggtcacacatttttttctagacactaaataaaattagtaaaattaaattaattataaaattatatatataatttactaactttagttagataaattaataatatataagttttagtacattaatattatattttaaat

SEQ ID NO: 2 - DNA, 4416 bp, Artificial Sequence
  Description of Artificial Sequence Synthetic polynucleotide
cacaattgacgtacatgagtctgagttccttgtttttgctaattatttcatccaatttattattcttgactatatcgagatcttttgtataggagtcagacttgtattcaacatgcttttctataatcattttagctatttcggcatcatccaatagtacattttccagattagcagaatagatattaatgtcgtatttgaacagagcctgtaacatctcaatgtctttattatctatagccaatttaatgtccggaatgaagagaagggaattattggtgtttgtcgacgtcatatagtcgagcaagagaatcatcatatccacgtgtccattttttatagtgatgtgaatacaactaaggagaatagccagatcaaaagtagatggtatctctgaaagaaagtaggaaacaatacttacatcattaagcatgacggcatgataaaatgaagttttccatccagttttcccatagaacatcagtctccaatttttcttaacaaacagttttaccgtttgcatgttaccactatcaaccgcataatacaatgcggtgtttcccttgtcatcaaattgtgaatcatccagtccactgaatagcaaaatctttactattttggtatcttccaatgtggctgcctgatgtaatggaaattcattctctagaagatttttcaatgctccagcgttcaacaacgtacatactagacgcacgttattatcagctattgcataatacaaggcactatgtccatggacatccgccttaaatgcatctttgctagagaggtctgaaacgagacgctaattagtgtatattttttcattttttataattttgtcatattgcaccagaattaataatatctctaatagatctgattagtagatacatggctatcgcataaaaattgcataaaaattttactttccaagtcggttcatctctatgtctgtataaatctgtcttttcttggtgtgctttaatttaatgcaaagatggataccaactcggagaaccaagaatagtccaatgattaaccctatgataaagaagaaagaggcaatagagcttttccaactactgaaccaaccttctacaagctcgattggatttttggatagcccagtatcaccaaagaataaactctcatcatcaggaagttgcgaagcagcgtcttgaatgtgaggatgttcgaacacctgagcctttgagctaagatgaagatcggagtccaacataccatgtccaatcatgtataaaggaaacttatatcctgaactggtcctcagaactccattgggtccaatttccacgtcttcatatggtgcccagtcatcccacagttccctttctgtggtagttccactgatcattccgaccattcttgagaggattggagcagcaatatcgactctgatgtatctggtctcaaagtattttagggtaccattgattatggtgaaagcaggaccggttcctgggtttttaggagcaagatagctgagatccactggagagattggaagacccgctctgattttgctccaggtttcttggcagagggaataatccaagatcctctcaacgtcctgaattagacttacatccactgaggtctgagatggagcagagatacttgacccttctgggcattcagggaatctggctgcagcaaagagatccttatcagccatctcgaaccagacacctgatgggagtctgactccccaatgcttgcagtattgcattttgcaggccttgcctccagtttcataagcaaagtagttacttctgaaccctgtgccctcctttcccagggatgatagctctccgtcctctgagaagaaggtgatgtccatggaaatgaggttagaatcacatagccctttgaccttatagtcagaatgccaggttgtagagttatggacagtggggcatatgtaattgctgcattttccgttgatgaactgtgaatcaacccattctcctgtgtattcatcaaccagcacatggtgaggagtcacctggacaatcactgcttcggcatccgtcacagttgcatatccacaactttgaggagggaagcctggattcagccaagttccttgtttcgtttgttcaatgctttccttgcattgttctacagatggagtgaaggatcggatggactgtgttatatacttcggtccataccagcggaaatcacaagtagtgacccatttggaagcatgacacatccaaccgtctgcttgaatagccttgtgactcttgggcattttgacttgtatggctgtgcctattaagtcattatgccaatttaaatctgagcttgacgggcaataatggtaattagaaggaacatttttccagtttcctttttggttgtgtgggaaaactatggtgaacttgcaattcaccccaatgaataagaaggctaagtacaaaaggcacttcattagaacgttatttatattccaaaaaaaaaaaataaaatttcaatttttgctagcgggccctaaggccataacttcgtatagcatacattatacgaagttataattctgcagatgaatttcattttgtttttttctatgctataaatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaataattaattaaaaggccgctcgaataacttcgtatagcatacattatacgaagttatgctagcacgatgctcttagaataacaacatatacacatttaataaaaataatatttattaagaaaattcagatttcacgtacccatcaatataaataaaataatgattccttacaccgtacccatattaaggagattccaccttacccataaacaatataaatccagtaatatcatgtctgatgatgaacacaaatggtgtattaaattccagtttttcaggagatgatctcgccgtagctaccataatagtagatgcctctgctacagttccttgttcgtcgacatctatctttgcattctgaaacattttataaatatataatgggtccctagtcatatgtttaaacgacgcattatctggattaaacatactaggagccatcatttcggctatcgacttaatatccctcttattttcgatagaaaatttagggagtttaagattgtacactttattccctaattgaaacgaccaatagtctaattttgcagccgtaatagaatctgtgaaatgggtcatattatcacctattgccaggtacatactaatattagcatccttatacggaaggcgtaccatatcatattcttcgtcatcgattgtgattgtatttccttgcaatttagtaactacgttcatcatgggaaccgttttcgtaccgtacttattagtaaaactagcattgcgtgttttagtgatatcaaacggatattgccatatacctttaaaatatatagtattaatgattgcccatagagtattattgtcgagcatattagaatctactacattagacataccggatctacgttctactatagaattaattttattaaccgcatctcgtctaaagtttaatctatataggccgaatctatgatattgttgataatacgacggtttaatgcacacagtattatctacgaaactttgataagttagatcagtgtacgtatatttagatgttttcagcttagctaatcctgatattaattctgtaaatgctggacccagatctctttttctcaaatccatag

SEQ ID NO: 3 - DNA, 1536 bp, Artificial Sequence
  Description of Artificial Sequence Synthetic polynucleotide
ttactttccaagtcggttcatctctatgtctgtataaatctgtcttttcttggtgtgctttaatttaatgcaaagatggataccaactcggagaaccaagaatagtccaatgattaaccctatgataaagaagaaagaggcaatagagcttttccaactactgaaccaaccttctacaagctcgattggatttttggatagcccagtatcaccaaagaataaactctcatcatcaggaagttgcgaagcagcgtcttgaatgtgaggatgttcgaacacctgagcctttgagctaagatgaagatcggagtccaacataccatgtccaatcatgtataaaggaaacttatatcctgaactggtcctcagaactccattgggtccaatttccacgtcttcatatggtgcccagtcatcccacagttccctttctgtggtagttccactgatcattccgaccattcttgagaggattggagcagcaatatcgactctgatgtatctggtctcaaagtattttagggtaccattgattatggtgaaagcaggaccggttcctgggtttttaggagcaagatagctgagatccactggagagattggaagacccgctctgattttgctccaggtttcttggcagagggaataatccaagatcctctcaacgtcctgaattagacttacatccactgaggtctgagatggagcagagatacttgacccttctgggcattcagggaatctggctgcagcaaagagatccttatcagccatctcgaaccagacacctgatgggagtctgactccccaatgcttgcagtattgcattttgcaggccttgcctccagtttcataagcaaagtagttacttctgaaccctgtgccctcctttcccagggatgatagctctccgtcctctgagaagaaggtgatgtccatggaaatgaggttagaatcacatagccctttgaccttatagtcagaatgccaggttgtagagttatggacagtggggcatatgtaattgctgcattttccgttgatgaactgtgaatcaacccattctcctgtgtattcatcaaccagcacatggtgaggagtcacctggacaatcactgcttcggcatccgtcacagttgcatatccacaactttgaggagggaagcctggattcagccaagttccttgtttcgtttgttcaatgctttccttgcattgttctacagatggagtgaaggatcggatggactgtgttatatacttcggtccataccagcggaaatcacaagtagtgacccatttggaagcatgacacatccaaccgtctgcttgaatagccttgtgactcttgggcattttgacttgtatggctgtgcctattaagtcattatgccaatttaaatctgagcttgacgggcaataatggtaattagaaggaacatttttccagtttcctttttggttgtgtgggaaaactatggtgaacttgcaattcaccccaatgaataagaaggctaagtacaaaaggcacttcat

SEQ ID NO: 4 - DNA, 4416 bp, Artificial Sequence; identical to SEQ ID NO: 2
  Description of Artificial Sequence Synthetic polynucleotide

SEQ ID NO: 5 - DNA, 4307 bp, Artificial Sequence
  Description of Artificial Sequence Synthetic polynucleotide
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaatttcttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttctcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactattcttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaa

SEQ ID NO: 6 - DNA, 8158 bp, Artificial Sequence
  Description of Artificial Sequence Synthetic polynucleotide
cacccgatgacaataatgacatggacgtagatattccgtattgtgcgacactagctaccgcaaataaaatatacggtagcgatagtatcgagttccacgcctccttcctacaaaaaataaaagacgattttcaaactgtaaactttaataatgctaaccaaacaaaggaactaatcaacgaatgggttaagacaatgacaaatggtaaaattaattccttattgactagtccgctatccattaatactcgtatgacagttgttagcgccgtccattttaaagcaatgtggaaatatccattttctaaacatcttacatatacagacaagttttatatttctaagaatatagttaccagtgttgatatgatggtgagcactgagaataacttgcaatatgtacatattaatgaattattcggaggattctctattatcgatattccatacgagggaaactctagtatggtaattatactaccggacgacatagaaggtatatataacatagaaaaaaatataacagatgaaaaatttaaaaaatggtgtggtatgttatctactaaaagtatagacttgtatatgccaaagtttaaagtggaaatgacagaaccgtataatctggtaccgattttagaaaatttaggacttactaatatattcggatattatgcagattttagcaagatgtgtaatgaaactatcactgtagaaaaatttctacatacgacgtttatagatgttaatgaggagtatacagaagcatcggccgttacaggagtatttatgactaacttttcgatggtatatcgtacgaaggtctacataaaccatccattcatgtacatgattaaagacaacacaggacgtatactttttatagggaaatactgctatccgcaataaatataaacaaatagactttttatcacgtttatctatgtctaaatattacaaatagtaatagtataaactaaagcctaggaaaaattgaaacattatattaatcagcgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagttcttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatcacgagactagcctcgagcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtcccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaataagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtccggactcagatctcgagctcaagcttcgaattctcgacctcgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggccgcggctcgagggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcggtacctttaagaccaatgacttacaaggcagctgtagatcttagccacttcttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcaaatttttatgtagtagttcatgtcatcttattattcagtatttataacttgcaaagaaatgaatatcagagagtgagaggaatttttataacttgtttattgcagcttataatggttacaaataaagcaatagcatcacaaatttcacaaataaagcatttttttcactgcattctagttgtggtttgtccaaactcatcaatgtatcttaggtatagcctctagaataacttcgtatagcatacattatacgaagttataattctgcagatggatcataaaaatatagtagaatttcattttgtttttttctatgctataaatagcttctcgagcatggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgatttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaaccgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaatggcatggacgagctgtacaagtaattaattaaggccgctcgaataacttcgtatagcatacattatacgaagttatctgataatacttaaaaaaataataatatcatttacaattaatagtataaactaaaaattaaacaaatcgttattataagtaatatcaaaatgatgatatacggattaatagcgtgtcttatattcgtgacttcatccatcgctagtccactttatattcccgttattccacccatttcggaagataaatcgttcaatagtgtagaggtattagtttccttgtttagagatgaccaaaaagactatacggtaacttctcagttcaataactacactatcgataccaaagactggactatcggcgtactatccacacctgatggtttggatataccattgactaatataacttattggtcacggtttactataggtcgtgcattgttcaaatcagagtctgaggatattttccaaaagaaaatgagtattctaggtgtttctatagaatgtaagaagtcgtcgacattacttacttttttgaccgtgcgtaaaatgactcgagtatttaataaatttccagatatggcttattatcgaggagactgtttaaaagccgtttatgtaacaatgacttataaaaatactaaaactggagagactgattacacgtacctctctaatggggggttgcctgcatactatcgtaatggggtcgatggttgattattgattagtatattccttattctttttattcacacaaaaagaacatttttataaacatgaaaccactgtctaaatgtaattatgatcttgatttatagatgaagatcagcctttagaggattttaacc

SEQ ID NO: 7 - RNA, 7 nt, Artificial Sequence
  Description of Artificial Sequence Synthetic oligonucleotide
  modified_base (6)..(6): a, c, u, g, unknown or other
uuuuunu

SEQ ID NO: 8 - DNA, 8 bp, Artificial Sequence
  Description of Artificial Sequence Synthetic oligonucleotide
atttttat

SEQ ID NO: 9 - DNA, 40 bp, Artificial Sequence
  Description of Artificial Sequence Synthetic oligonucleotide
tatttatattccaaaaaaaaaaaataaaatttcaattttt

SEQ ID NO: 10 - DNA, 40 bp, Artificial Sequence
  Description of Artificial Sequence Synthetic oligonucleotide
aaaaattgaaattttattttttttttttggaatataaata

SEQ ID NO: 11 - DNA, 204 bp, Artificial Sequence
  Description of Artificial Sequence Synthetic polynucleotide
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 12 - DNA, 511 bp, Artificial Sequence
  Description of Artificial Sequence Synthetic polynucleotide
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 13 - DNA, 5678 bp, Artificial Sequence
  Description of Artificial Sequence Synthetic polynucleotide
catttatacatattcctggagatacactgtttaaaattagcgaggtatttattcataataagataaatccatcttccagaatggcatacatagaagaagaaaagagaaatttagttactgctatacagatgtatactcctcctgatggatcatccggtatagtctttgtggcatccgttgcacagtgtcacgaatataaatcatatttagaaaaaagattaccgtatgatatgtatattattcatggtaaggtcttagatatagacgaaatattagaaaaagtgtattcatcacctaatgtatcgataattatttctactccttatttggaatccagcgttactatacgcaatgttacacacatttatgatatgggtaaagtttttgtccccgctccttttggaggatcgcaagaatttatttctaaatctatgagagatcaacgaaaaggaagagtaggaagagttaatcctggtacatacgtctatttctatgatctgtcttatatgaagtctatacagcgaatagattcagaatttctacataattatatattgtacgctaataagtttaatctaacactccccgaagatttgtttataatccctacaaatttggatattctatggcgtacaaaggaatatatagactcgttcgatattagtacagaaacatggaataaattattatccaattattatatgaagatgatagagtatgctaaactttatgtactaagtcctattctcgctgaggagttggataactttgagaggacgggagaattaactagtattgtacgagaagccattttatctctaaatttacgaattaagattttaaattttaaacataaagatgatgatacgtatatacacttttgtaaaatattattcggtgtctataacggaacaaacgctactatatattatcatagacctctaacgggatatatgaatatgatttcagatactatatttgttcctgtagataataactaaaaggccggccgctagctcactaattccaaacccacccgctttttatagtaagtttttcacccataaataataaatacaataattaatttctcgtaaaagtagaaaatatattctaatttattgcacggtaaggaagtagatcataaggatccatgaacacaattaacatagccaagaacgacttcagtgacattgagttggctgccatcccgtttaataccttagcagatcactacggagagcgacttgcgagagagcaattggcccttgagcatgaatcctacgaaatgggagaagcgcgattccgtaagatgttcgagagacaattaaaagccggtgaagtagcggacaacgccgcagcaaagccacttatcacaacactacttccgaagatgatagcacgtattaacgattggtttgaggaagtaaaggccaagagaggtaagagaccgacggctttccaattcctacaagagatcaagcctgaagcggtggcgtatataactatcaaaacgaccctagcgtgcctaacttctgccgataacacgacggtacaggctgtcgcttcagcgattggaagagcgatagaagatgaagcacgtttcggacgaatccgtgatctagaagctaaacattttaaaaagaacgttgaggagcagttgaacaaacgagtaggtcacgtgtacaaaaaggcattcatgcaagtggtagaagccgacatgctatccaagggattgcttggaggtgaggcctggtcgagttggcacaaagaggactctatccacgtcggtgttagatgcatagagatgctaatagagtcgacaggtatggtttccctacatcgtcaaaatgctggagttgtaggtcaggatagtgaaacgatcgagctagcgccggaatacgcagaggcgattgcgacgcgagctggtgcccttgctggaatttcaccgatgttccaaccgtgcgtggtaccgccaaaaccctggacaggaattacgggtggtggttattgggccaacggacgtcgaccacttgcattggtacgaacacattcgaagaaagcattaatgcgttacgaagacgtatacatgccggaagtatacaaagcaattaatatcgctcagaataccgcatggaaaatcaacaaaaaggtattggcagtggctaacgtaataaccaagtggaaacattgtcctgttgaagacattcctgctattgaacgagaagaactacctatgaagcctgaggacatagacatgaatccggaagcattgaccgcgtggaaacgagctgcggctgctgtctaccgaaaggacaaggcacgaaaatccagacgaatatcgctagaatttatgttggaacaagccaataagtttgcaaaccacaaggcgatatggtttccatacaatatggattggcgaggaagagtttacgctgtgtctatgttcaacccacagggaaacgacatgacaaaaggactattaacattagcaaaaggaaagcctatcggaaaagaaggttactattggttgaagatacacggagctaattgcgcaggtgtcgataaagtccccttcccggaacgtataaaattcatagaggagaaccacgaaaacatcatggcgtgcgccaaatccccgctagaaaatacttggtgggcggaacaagattctccattttgcttcttagcattctgctttgaatacgcaggagtccagcaccacggactaagttacaattgttcgcttcctctagcatttgacggttcgtgcagtggaatacaacatttctcagccatgctaagagatgaggtgggaggtcgtgcggtaaacctattgccatcggagaccgtgcaggatatatatggtatcgtcgccaagaaagtaaacgagattctacaggcggatgccattaacggaaccgacaatgaagttgtcactgttaccgacgagaacacaggtgaaatttcagaaaaggtgaagcttggaactaaagcattagccggacagtggctagcgtatggtgtcactcgtagtgtcaccaaacgtagtgtcatgacgcttgcatatggttccaaagaattcggttttagacagcaggtcttggaggacacgattcaacccgccatagattcgggtaaaggattgatgttcacacagcctaatcaggctgccggttatatggccaaactaatttgggagtctgttagtgttacagtcgttgccgctgtcgaagccatgaattggttgaaaagtgccgcaaaactacttgcggccgaagtgaaggataagaagactggtgagatcctacgaaagagatgcgctgtgcattgggtcactccggatggttttccggtctggcaagaatataaaaagccgatacaaacgcgtttgaacttaatgtttttaggacaattcagattacaacccaccattaacacgaataaagattcggaaattgacgcacacaagcaagaatcaggtattgcaccaaacttcgtgcactcacaagatggttctcacttaagaaaaacggttgtctgggctcatgagaagtatggtatagagagttttgcattgattcatgactccttcggtactataccagccgacgccgccaatttgttcaaagcggtccgagaaaccatggtagacacttacgaatcttgcgacgtccttgcggacttttatgatcaattcgctgatcagttacatgagtcgcaattagacaagatgccggcattaccggcgaagggaaacttgaacttgagagatattttagaatctgatttcgcctttgcctaaaagcttataacttcgtatagcatacattatacgaagttataattctgcagatggatcataaaaatatagtagaatttcattttgtttttttctatgctataaatagcttctcgagcatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtcccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaataataaataacttcgtatagcatacattatacgaagttatatcaaactctaatgaccacatctttttttagagatgaaaaattttctacatctccttttgtagacacgactaaacattttgcaaaaaaaagtttattagtgtttagataatcgtatacttcatcagtgtagatagtaaatgtgaacaaataaaaggtattcttactcaatagattggtaaattccatagaatatattaatcctttcttcttgagatcccacatcatttcaaccagagacgttttatccaatgatttacctcgtactataccacatacaaaactagattttgcagtgacgtcgtacctggtattcctaccaaacaaaattttacttttagttcttttagaaaattctaaggtagaatctctatttgccaatatgtcatctatggaattaccactagcaaaaaatgatagaaatatatattgatacatcgcagctggttttgatctactatactttaaaaacgaatcagattccataattgcctgtatatcatcagctgaaaaactatgttttacacgtattccttcggcatttctttttaatgatatatcttgtttagacaatgataaagttatcatgtccatgagagacgcgtctccgtatcgtataaatatttcattagatgttagacgcttcattaggggtatacttctataaggtttcttaatcagtccatcattggttgcgtcaagaactactatcggatgttgttgggtatctctagtgttacacatggccttactaaagtttgggtaaataactatgatatctctattaattatagatgcatatatttcattcgtcaaggatattagtatcgacttgctatcgtcattaatacgtgtaatgtaatcatataaatcatgcgatagccaaggaaaattcaaatagatgttcatcatataatcgtcgctataattcatattaatacgttgacattgactaatttgtaatatagcctcgccacgaagaaagctctcgtattcagtttcatc

SEQ ID NO: 14 - DNA, 2652 bp, Artificial Sequence
  Description of Artificial Sequence Synthetic polynucleotide
atgaacacaattaacatagccaagaacgacttcagtgacattgagttggctgccatcccgtttaataccttagcagatcactacggagagcgacttgcgagagagcaattggcccttgagcatgaatcctacgaaatgggagaagcgcgattccgtaagatgttcgagagacaattaaaagccggtgaagtagcggacaacgccgcagcaaagccacttatcacaacactacttccgaagatgatagcacgtattaacgattggtttgaggaagtaaaggccaagagaggtaagagaccgacggctttccaattcctacaagagatcaagcctgaagcggtggcgtatataactatcaaaacgaccctagcgtgcctaacttctgccgataacacgacggtacaggctgtcgcttcagcgattggaagagcgatagaagatgaagcacgtttcggacgaatccgtgatctagaagctaaacattttaaaaagaacgttgaggagcagttgaacaaacgagtaggtcacgtgtacaaaaaggcattcatgcaagtggtagaagccgacatgctatccaagggattgcttggaggtgaggcctggtcgagttggcacaaagaggactctatccacgtcggtgttagatgcatagagatgctaatagagtcgacaggtatggtttccctacatcgtcaaaatgctggagttgtaggtcaggatagtgaaacgatcgagctagcgccggaatacgcagaggcgattgcgacgcgagctggtgcccttgctggaatttcaccgatgttccaaccgtgcgtggtaccgccaaaaccctggacaggaattacgggtggtggttattgggccaacggacgtcgaccacttgcattggtacgaacacattcgaagaaagcattaatgcgttacgaagacgtatacatgccggaagtatacaaagcaattaatatcgctcagaataccgcatggaaaatcaacaaaaaggtattggcagtggctaacgtaataaccaagtggaaacattgtcctgttgaagacattcctgctattgaacgagaagaactacctatgaagcctgaggacatagacatgaatccggaagcattgaccgcgtggaaacgagctgcggctgctgtctaccgaaaggacaaggcacgaaaatccagacgaatatcgctagaatttatgttggaacaagccaataagtttgcaaaccacaaggcgatatggtttccatacaatatggattggcgaggaagagtttacgctgtgtctatgttcaacccacagggaaacgacatgacaaaaggactattaacattagcaaaaggaaagcctatcggaaaagaaggttactattggttgaagatacacggagctaattgcgcaggtgtcgataaagtccccttcccggaacgtataaaattcatagaggagaaccacgaaaacatcatggcgtgcgccaaatccccgctagaaaatacttggtgggcggaacaagattctccattttgcttcttagcattctgctttgaatacgcaggagtccagcaccacggactaagttacaattgttcgcttcctctagcatttgacggttcgtgcagtggaatacaacatttctcagccatgctaagagatgaggtgggaggtcgtgcggtaaacctattgccatcggagaccgtgcaggatatatatggtatcgtcgccaagaaagtaaacgagattctacaggcggatgccattaacggaaccgacaatgaagttgtcactgttaccgacgagaacacaggtgaaatttcagaaaaggtgaagcttggaactaaagcattagccggacagtggctagcgtatggtgtcactcgtagtgtcaccaaacgtagtgtcatgacgcttgcatatggttccaaagaattcggttttagacagcaggtcttggaggacacgattcaacccgccatagattcgggtaaaggattgatgttcacacagcctaatcaggctgccggttatatggccaaactaatttgggagtctgttagtgttacagtcgttgccgctgtcgaagccatgaattggttgaaaagtgccgcaaaactacttgcggccgaagtgaaggataagaagactggtgagatcctacgaaagagatgcgctgtgcattgggtcactccggatggttttccggtctggcaagaatataaaaagccgatacaaacgcgtttgaacttaatgtttttaggacaattcagattacaacccaccattaacacgaataaagattcggaaattgacgcacacaagcaagaatcaggtattgcaccaaacttcgtgcactcacaagatggttctcacttaagaaaaacggttgtctgggctcatgagaagtatggtatagagagttttgcattgattcatgactccttcggtactataccagccgacgccgccaatttgttcaaagcggtccgagaaaccatggtagacacttacgaatcttgcgacgtccttgcggacttttatgatcaattcgctgatcagttacatgagtcgcaattagacaagatgccggcattaccggcgaagggaaacttgaacttgagagatattttagaatctgatttcgcctttgcctaa